SEQUENCE LISTING (265 sequences)

SEQ ID NO: 1 - protein, 10 aa, synthetic construct
  REGION 1..10: note = Description of Artificial Sequence: Synthetic peptide
GASISSFYWS

SEQ ID NO: 2 - protein, 9 aa, synthetic construct
  REGION 1..9: note = Description of Artificial Sequence: Synthetic peptide
RISPSGKTN

SEQ ID NO: 3 - protein, 13 aa, synthetic construct
  REGION 1..13: note = Description of Artificial Sequence: Synthetic peptide
GEYSGTYSYSFDV

SEQ ID NO: 4 - protein, 17 aa, synthetic construct
  REGION 1..17: note = Description of Artificial Sequence: Synthetic peptide
RSSESLLDSEDGNTYLD

SEQ ID NO: 5 - protein, 7 aa, synthetic construct
  REGION 1..7: note = Description of Artificial Sequence: Synthetic peptide
TLSYRAS

SEQ ID NO: 6 - protein, 9 aa, synthetic construct
  REGION 1..9: note = Description of Artificial Sequence: Synthetic peptide
MQRMEFPLT

SEQ ID NO: 7 - protein, 12 aa, synthetic construct
  REGION 1..12: note = Description of Artificial Sequence: Synthetic peptide
GDSVSSNSATWN

SEQ ID NO: 8 - protein, 11 aa, synthetic construct
  REGION 1..11: note = Description of Artificial Sequence: Synthetic peptide
RTYYRSKWYND

SEQ ID NO: 9 - protein, 7 aa, synthetic construct
  REGION 1..7: note = Description of Artificial Sequence: Synthetic peptide
VDIAFDY

SEQ ID NO: 10 - protein, 13 aa, synthetic construct
  REGION 1..13: note = Description of Artificial Sequence: Synthetic peptide
SGSSSNIGNHGVN

SEQ ID NO: 11 - protein, 7 aa, synthetic construct
  REGION 1..7: note = Description of Artificial Sequence: Synthetic peptide
NDDLLPS

SEQ ID NO: 12 - protein, 11 aa, synthetic construct
  REGION 1..11: note = Description of Artificial Sequence: Synthetic peptide
AAWDDSLNGVV

SEQ ID NO: 13 - protein, 12 aa, synthetic construct
  REGION 1..12: note = Description of Artificial Sequence: Synthetic peptide
GDSVSNNSATWN

SEQ ID NO: 14 - protein, 10 aa, synthetic construct
  REGION 1..10: note = Description of Artificial Sequence: Synthetic peptide
GVSISNYYWS

SEQ ID NO: 15 - protein, 9 aa, synthetic construct
  REGION 1..9: note = Description of Artificial Sequence: Synthetic peptide
RISPSGRTN

SEQ ID NO: 16 - protein, 13 aa, synthetic construct
  REGION 1..13: note = Description of Artificial Sequence: Synthetic peptide
GEYSGTYSYSFDI

SEQ ID NO: 17 - protein, 17 aa, synthetic construct
  REGION 1..17: note = Description of Artificial Sequence: Synthetic peptide
RSSQSLFDSDDGNTYLD

SEQ ID NO: 18 - protein, 12 aa, synthetic construct
  REGION 1..12: note = Description of Artificial Sequence: Synthetic peptide
GDSVSSNSAAWN

SEQ ID NO: 19 - protein, 7 aa, synthetic construct
  REGION 1..7: note = Description of Artificial Sequence: Synthetic peptide
VNTTFDY

SEQ ID NO: 20 - protein, 13 aa, synthetic construct
  REGION 1..13: note = Description of Artificial Sequence: Synthetic peptide
SGSSSNIGKNAVS

SEQ ID NO: 21 - protein, 7 aa, synthetic construct
  REGION 1..7: note = Description of Artificial Sequence: Synthetic peptide
SDDLLSS

SEQ ID NO: 22 - protein, 10 aa, synthetic construct
  REGION 1..10: note = Description of Artificial Sequence: Synthetic peptide
GASISSYYWS

SEQ ID NO: 23 - protein, 9 aa, synthetic construct
  REGION 1..9: note = Description of Artificial Sequence: Synthetic peptide
RISNTGRTN

SEQ ID NO: 24 - protein, 13 aa, synthetic construct
  REGION 1..13: note = Description of Artificial Sequence: Synthetic peptide
GEYSGTFSYGFDI

SEQ ID NO: 25 - protein, 17 aa, synthetic construct
  REGION 1..17: note = Description of Artificial Sequence: Synthetic peptide
RSSLSLLDSDDGKIYLD

SEQ ID NO: 26 - protein, 9 aa, synthetic construct
  REGION 1..9: note = Description of Artificial Sequence: Synthetic peptide
RIYSNGNIN

SEQ ID NO: 27 - protein, 13 aa, synthetic construct
  REGION 1..13: note = Description of Artificial Sequence: Synthetic peptide
GEYSGDFSYSFDI

SEQ ID NO: 28 - protein, 17 aa, synthetic construct
  REGION 1..17: note = Description of Artificial Sequence: Synthetic peptide
RSSQSLLDSDDGNTYLD

SEQ ID NO: 29 - protein, 9 aa, synthetic construct
  REGION 1..9: note = Description of Artificial Sequence: Synthetic peptide
MQRIEFPLT

SEQ ID NO: 30 - protein, 10 aa, synthetic construct
  REGION 1..10: note = Description of Artificial Sequence: Synthetic peptide
GGSISNYYWS

SEQ ID NO: 31 - protein, 9 aa, synthetic construct
  REGION 1..9: note = Description of Artificial Sequence: Synthetic peptide
RIFYSGKTN

SEQ ID NO: 32 - protein, 13 aa, synthetic construct
  REGION 1..13: note = Description of Artificial Sequence: Synthetic peptide
GEYSGEYSYSFDI

SEQ ID NO: 33 - protein, 17 aa, synthetic construct
  REGION 1..17: note = Description of Artificial Sequence: Synthetic peptide
RSSQSLLDSDDGNTYVD

SEQ ID NO: 34 - DNA, 330 bp, synthetic construct
  misc_feature 1..330: note = Description of Artificial Sequence: Synthetic polynucleotide
  source 1..330: mol_type = other DNA
cagacagtggtcacccagcctccatctgtgtctgaggcccctagacagagagtgaccatctcctgctccggctcctcctccaacatcggcaatcatggcgtgaactggtatcagcagctgcccggcaaggctcccaaactgctgatctacaacgacgacctgctgccttctggcgtgtccgacagattctccggctctacctctggcacctctggatccctggctatctctggcctgcagtctgaggacgaggccgactactattgtgccgcctgggacgattctctgaacggcgttgtgtttggcggaggcaccaagctgacagtgttg

SEQ ID NO: 35 - protein, 121 aa, synthetic construct
  REGION 1..121: note = Description of Artificial Sequence: Synthetic polypeptide
QVQLQESGPGLVKPSETLSLTCSVSGASISSFYWSWIRQPADEGLEWIGRISPSGKTNYIPSLKSRIIMSLDASKNQFSLRLNSVTAADTAMYYCARGEYSGTYSYSFDVWGQGTMVTVSS

SEQ ID NO: 36 - DNA, 363 bp, synthetic construct
  misc_feature 1..363: note = Description of Artificial Sequence: Synthetic polynucleotide
  source 1..363: mol_type = other DNA
caggttcagctgcaagagtctggtcctggcctggtcaagccttccgagacactgtctctgacctgctctgtgtccggcgcctccatctcttccttctactggtcctggatccggcagcctgctgacgaaggactggaatggatcggccggatcagcccttctggcaagaccaactacatccccagcctgaagtcccggatcatcatgtccctggacgcctccaagaaccagttctccctgcggctgaactctgtgaccgctgccgataccgccatgtactactgtgccagaggcgagtactccggcacctactcctacagctttgacgtgtggggacaaggcaccatggtcacagtttcttct

SEQ ID NO: 37 - protein, 113 aa, synthetic construct
  REGION 1..113: note = Description of Artificial Sequence: Synthetic polypeptide
DIVMTQSPLSLSVTPGEPASISCRSSESLLDSEDGNTYLDWFLQKPGQSPQLLIYTLSYRASGVPDRFSGSGSDTDFTLHISSLEAEDVGLYYCMQRMEFPLTFGQGTKVEIK

SEQ ID NO: 38 - DNA, 339 bp, synthetic construct
  misc_feature 1..339: note = Description of Artificial Sequence: Synthetic polynucleotide
  source 1..339: mol_type = other DNA
gacatcgtgatgacccagtctccactgagcctgtctgtgacacctggcgagcctgcctccatctcctgtagatcttctgagtccctgctggacagcgaggacggcaatacctacctggactggttcctgcagaagcccggacagtctcctcagctgctgatctacaccctgtcctacagagcctctggcgtgcccgatagattctccggctctggctctgacaccgactttaccctgcacatctccagcctggaagccgaggatgtgggcctgtactactgtatgcagcggatggaatttcccctgaccttcggccagggcaccaaggtggaaatcaag

SEQ ID NO: 39 - protein, 119 aa, synthetic construct
  REGION 1..119: note = Description of Artificial Sequence: Synthetic polypeptide
QVQLQQSGPGLVKPSQTLSLTCAISGDSVSNNSATWNWIRQSPSRGLEWLGRTYYRSKWYNDYTVSVKSRITINPDTSKNQFSLQLNSVTPEDTAVYYCTRVDIAFDYWGQGTLVTVSS

SEQ ID NO: 40 - DNA, 357 bp, synthetic construct
  misc_feature 1..357: note = Description of Artificial Sequence: Synthetic polynucleotide
  source 1..357: mol_type = other DNA
caagtgcaactgcagcagtctggccctggactggtcaagccttctcagaccctgtctctgacctgcgccatctccggcgactccgtgtccaacaactccgctacctggaactggatcagacagtccccttccagaggcctggaatggctgggcagaacctactaccggtccaagtggtacaacgactacaccgtgtccgtgaagtcccggatcaccatcaaccctgatacctctaagaaccagttctccctgcaactgaactctgtgacccctgaggacaccgccgtgtactactgcaccagagtggacatcgccttcgactactggggccagggcaccctggtgaccgtgtctagc

SEQ ID NO: 41 - protein, 110 aa, synthetic construct
  REGION 1..110: note = Description of Artificial Sequence: Synthetic polypeptide
QTVVTQPPSVSEAPRQRVTISCSGSSSNIGNHGVNWYQQLPGKAPKLLIYNDDLLPSGVSDRFSGSTSGTSGSLAISGLQSEDEADYYCAAWDDSLNGVVFGGGTKLTVL

SEQ ID NO: 42 - DNA, 330 bp, synthetic construct
  misc_feature 1..330: note = Description of Artificial Sequence: Synthetic polynucleotide
  source 1..330: mol_type = other DNA
cagactgtggtgactcagccaccctcggtgtctgaagcccccaggcagagggtcaccatctcctgttctggaagtagctccaacatcggaaatcatggtgtaaactggtaccagcagctcccaggaaaggctcccaaactcctcatctataatgatgatctgctgccctcaggggtctctgaccgattctctggctccacgtctggcacctcaggttccctggccatcagtgggctccagtctgaggatgaggctgattattactgtgcagcatgggatgacagcctgaatggtgtggtattcggcggagggactaaactgaccgtccta

SEQ ID NO: 43 - protein, 119 aa, synthetic construct
  REGION 1..119: note = Description of Artificial Sequence: Synthetic polypeptide
QVQLQQSGPGLVKPSQTLSLTCAISGDSVSSNSATWNWIRQSPSRGLEWLGRTYYRSKWYNDYTVSVKSRITINPDTSKNQFSLQLNSVTPEDTAVYYCTRVDIAFDYWGQGTLVTVSS

SEQ ID NO: 44 - DNA, 357 bp, synthetic construct
  misc_feature 1..357: note = Description of Artificial Sequence: Synthetic polynucleotide
  source 1..357: mol_type = other DNA
caggttcagctgcagcagtctggccctggactggtcaagccctctcagaccctgtctctgacctgtgccatctccggcgactccgtgtcctctaattctgccacctggaactggatccggcagtcccctagtagaggcctggaatggctgggcagaacctactaccggtccaagtggtacaacgactacaccgtgtccgtgaagtcccggatcaccatcaatcccgacacctccaagaaccagttctccctgcagctcaacagcgtgacccctgaggataccgccgtgtactactgcaccagagtggatatcgccttcgactactggggccagggcacactggttaccgtttcttct

SEQ ID NO: 45 - protein, 121 aa, synthetic construct
  REGION 1..121: note = Description of Artificial Sequence: Synthetic polypeptide
QVQLQESGPGLVKPSQTLSLTCTVSGVSISNYYWSWIRQPPGKGLEWIGRISPSGRTNYNPSLKSRVTMSLDASKNQFSLKLSSVTAADTAVYYCARGEYSGTYSYSFDIWGQGTMVTVSS

SEQ ID NO: 46 - DNA, 363 bp, synthetic construct
  misc_feature 1..363: note = Description of Artificial Sequence: Synthetic polynucleotide
  source 1..363: mol_type = other DNA
caggttcagctgcaagagtctggccctggcctggtcaagccctctcagaccctgtctctgacctgtaccgtgtccggcgtgtccatctccaactactactggtcctggatccggcagcctcctggcaaaggactggaatggatcggccgcatctctccttctggtcgcaccaactacaaccccagcctgaaaagcagagtgaccatgtctctggacgcctccaagaaccagttctccctgaagctgtcctccgtgaccgctgctgataccgccgtgtactactgtgccagaggcgagtactccggcacctactcctacagcttcgacatctggggccagggcaccatggtcacagtctcttct

SEQ ID NO: 47 - protein, 113 aa, synthetic construct
  REGION 1..113: note = Description of Artificial Sequence: Synthetic polypeptide
DIQMTQSPSSLSASVGDRVTITCRSSQSLFDSDDGNTYLDWFQQKPGQSPKLLIQTLSYRASGVPSRFSGSGSGTDFTLTISSLQPEDFATYYCMQRMEFPLTFGGGTKVEIK

SEQ ID NO: 48 - DNA, 339 bp, synthetic construct
  misc_feature 1..339: note = Description of Artificial Sequence: Synthetic polynucleotide
  source 1..339: mol_type = other DNA
gacatccagatgacccagtctccatcctctctgtccgcctctgtgggcgacagagtgaccatcacctgtcggtcctctcagtccctgttcgactctgacgacggcaacacctacctggactggttccagcagaagcccggccagtctcctaagctgctgatccagacactgtcctacagagcctctggcgtgccctccagattttccggctctggctctggcaccgactttaccctgacaatctccagcctgcagcctgaggacttcgccacctactactgtatgcagcggatggaatttcccctgaccttcggcggaggcaccaaggtggaaatcaag

SEQ ID NO: 49 - protein, 119 aa, synthetic construct
  REGION 1..119: note = Description of Artificial Sequence: Synthetic polypeptide
EVQLVESGPGLVKPSQTLSLTCAISGDSVSSNSAAWNWIRQSPSRGLEWLGRTYYRSKWYNDYAVSVRSRITINPDTSKNQFSLQLNSVTPEDTAVYFCTRVNTTFDYWGQGTLVTVSS

SEQ ID NO: 50 - DNA, 357 bp, synthetic construct
  misc_feature 1..357: note = Description of Artificial Sequence: Synthetic polynucleotide
  source 1..357: mol_type = other DNA
gaagtgcaactggtggaatctggccctggactggtgaagccttctcagaccctgtctctgacctgcgccatctccggcgactccgtgtcctccaactctgccgcctggaactggatcagacagtccccttccagaggcctggaatggctgggcagaacctactacagatccaagtggtacaacgactacgccgtgtctgtgcggtcccggatcaccatcaaccctgacacctctaagaaccagttctccctgcaactgaactccgtgacccctgaggacaccgccgtgtacttctgcaccagagtgaacaccaccttcgactactggggccagggcaccctggtcaccgtgtcctct

SEQ ID NO: 51 - protein, 110 aa, synthetic construct
  REGION 1..110: note = Description of Artificial Sequence: Synthetic polypeptide
QSVLTQPPSVSEAPRQRVTISCSGSSSNIGKNAVSWYQQLPGKAPKLLIHSDDLLSSGVSDRFSGSQSGTSASLAISGLQSEDEADYYCAAWDDSLNGVVFGGGTKLTVL

SEQ ID NO: 52 - DNA, 330 bp, synthetic construct
  misc_feature 1..330: note = Description of Artificial Sequence: Synthetic polynucleotide
  source 1..330: mol_type = other DNA
cagtctgtgctgactcagccaccctcggtgtctgaagcccccaggcagagggtcaccatctcctgttctggaagtagctccaacatcggaaaaaatgctgtaagctggtaccagcagctcccaggaaaggctcccaaactcctcatccattctgatgatctgctgtcctcaggggtctctgaccgattctctggctcccagtctggcacctcagcctccctggccatcagtgggctccagtccgaggatgaggctgattattactgtgcagcatgggatgacagcctgaatggtgtggtattcggcggagggactaaactgaccgtccta

SEQ ID NO: 53 - protein, 110 aa, synthetic construct
  REGION 1..110: note = Description of Artificial Sequence: Synthetic polypeptide
QLVLTQPPSVSEAPRQRVTISCSGSSSNIGNHGVNWYQQLPGKAPKLLIYNDDLLPSGVSDRFSGSTSGTSGSLAISGLQSEDEADYYCAAWDDSLNGVVFGGGTKLTVL

SEQ ID NO: 54 - DNA, 330 bp, synthetic construct
  misc_feature 1..330: note = Description of Artificial Sequence: Synthetic polynucleotide
  source 1..330: mol_type = other DNA
cagcttgtgctgactcagccaccctcggtgtctgaagcccccaggcagagggtcaccatctcctgttctggaagtagctccaacatcggaaatcatggtgtaaactggtaccagcagctcccaggaaaggctcccaaactcctcatctataatgatgatctgctgccctcaggggtctctgaccgattctctggctccacgtctggcacctcaggttccctggccatcagtgggctccagtctgaggatgaggctgattattactgtgcagcatgggatgacagcctgaatggtgtggtattcggcggagggactaaactgaccgtccta

SEQ ID NO: 55 - protein, 119 aa, synthetic construct
  REGION 1..119: note = Description of Artificial Sequence: Synthetic polypeptide
QVQLQQSGPGLVKPSQTLSLTCAISGDSVSNNSATWNWIRQSPSRGLEWLGRTYYRSKWYNDYTVSVKSRITINPDTSKNQFSLQLNSVTPEDTAVYYCTRVDIAFDYWGQGTTVTVSS

SEQ ID NO: 56 - DNA, 357 bp, synthetic construct
  misc_feature 1..357: note = Description of Artificial Sequence: Synthetic polynucleotide
  source 1..357: mol_type = other DNA
caagtgcaactgcagcagtctggccctggcctggtgaagccttctcagaccctgtctctgacctgcgccatctccggcgactccgtgtccaacaactctgctacctggaactggatcagacagtccccttccagaggcctggaatggctgggcagaacctactacagatccaagtggtacaacgactacaccgtgtctgtgaagtcccggatcaccatcaaccccgatacctctaagaaccagttctccctgcaactgaactccgtgacccctgaggacaccgccgtgtactactgcaccagagtggacatcgccttcgactactggggccagggcaccaccgtgacagtgtcctcc

SEQ ID NO: 57 - protein, 110 aa, synthetic construct
  REGION 1..110: note = Description of Artificial Sequence: Synthetic polypeptide
QSALTQPPSVSEAPRQRVTISCSGSSSNIGNHGVNWYQQLPGKAPKLLIYNDDLLPSGVSDRFSGSTSGTSGSLAISGLQSEDEADYYCAAWDDSLNGVVFGGGTKLTVL

SEQ ID NO: 58 - DNA, 330 bp, synthetic construct
  misc_feature 1..330: note = Description of Artificial Sequence: Synthetic polynucleotide
  source 1..330: mol_type = other DNA
cagtctgccctgactcagccaccctcggtgtctgaagcccccaggcagagggtcaccatctcctgttctggaagtagctccaacatcggaaatcatggtgtaaactggtaccagcagctcccaggaaaggctcccaaactcctcatctataatgatgatctgctgccctcaggggtctctgaccgattctctggctccacgtctggcacctcaggttccctggccatcagtgggctccagtctgaggatgaggctgattattactgtgcagcatgggatgacagcctgaatggtgtggtattcggcggagggaccaagctgaccgtccta

SEQ ID NO: 59 - protein, 121 aa, synthetic construct
  REGION 1..121: note = Description of Artificial Sequence: Synthetic polypeptide
QVQLVQSGPGLVKPSETLSLICTVSGASISSYYWSWIRQPAGKGLEWLGRISNTGRTNYNPSLKSRVTMSSDTSKNQFSLKLRSVTAADTAVYYCARGEYSGTFSYGFDIWGQGTMVTVSS

SEQ ID NO: 60 - DNA, 363 bp, synthetic construct
  misc_feature 1..363: note = Description of Artificial Sequence: Synthetic polynucleotide
  source 1..363: mol_type = other DNA
caagtgcagttggtacagtctggtcccgggcttgtaaagccttctgaaacattgagcctgatatgcaccgtctccggtgccagtataagtagttattactggtcatggatccgtcagcccgcaggtaaaggcttggagtggttgggaaggattagtaatactggacgaaccaattacaatccttccctgaagagtcgtgttaccatgagtagtgataccagcaagaaccagttctcacttaaattgaggtccgtgaccgccgctgacaccgctgtctactactgtgctcgcggagagtattcaggaaccttttcatacgggttcgatatttggggccaggggacaatggttactgtgagttca

SEQ ID NO: 61 - protein, 113 aa, synthetic construct
  REGION 1..113: note = Description of Artificial Sequence: Synthetic polypeptide
EIVLTQSPLSLSVTPGEPASISCRSSLSLLDSDDGKIYLDWYLQRPGQSPQLLIQTLSYRASGVPDRFSGSGSGTDHTLKISRVEAEDVGVYYCMQRMEFPLTFGGGTKLEIK

SEQ ID NO: 62 - DNA, 339 bp, synthetic construct
  misc_feature 1..339: note = Description of Artificial Sequence: Synthetic polynucleotide
  source 1..339: mol_type = other DNA
gaaattgtgttgacacagtctccactctccctgtccgtcacccctggagagccggcctccatctcctgcaggtctagtctgagcctcttggatagtgatgatggaaaaatctatttggactggtacctgcagaggccagggcagtctccacagctcctgatccagacgctttcctatcgggcctctggagtcccagacaggttcagtggcagtgggtcaggcactgatcacacactgaaaatcagcagggtggaggctgaggatgttggagtttattactgcatgcaacgtatggagtttccgctcactttcggcggagggaccaagctggagatcaaa

SEQ ID NO: 63 - protein, 121 aa, synthetic construct
  REGION 1..121: note = Description of Artificial Sequence: Synthetic polypeptide
QVQLQESGPGLVKPSETLSLTCSVSGASISSYYWSWIRQPAGKGLEWIGRIYSNGNINYHSSLKRRVTMSVDTSKNQFSLKLNAVTAADTAVYYCARGEYSGDFSYSFDIWGQGTMVTVSS

SEQ ID NO: 64 - DNA, 363 bp, synthetic construct
  misc_feature 1..363: note = Description of Artificial Sequence: Synthetic polynucleotide
  source 1..363: mol_type = other DNA
caagtacagcttcaagagtccgggccaggtctcgttaagccatccgaaactctgtcacttacttgttcagtctcaggagcttcaatttcttcatattactggtcctggattcgtcaaccagccggcaaaggtttggagtggataggccggatatattcaaatggaaatatcaactaccactcatcccttaaacgtagggttacaatgagtgtggatacctctaagaatcagttcagtttgaaattgaatgctgtcaccgccgctgacaccgcagtctattattgtgccagaggcgaatacagtggtgacttctcatatagctttgacatttggggtcagggaacaatggtcacagtgagttcc

SEQ ID NO: 65 - protein, 113 aa, synthetic construct
  REGION 1..113: note = Description of Artificial Sequence: Synthetic polypeptide
DIVMTQSPLSLPVTPGEPASISCRSSQSLLDSDDGNTYLDWFLQKPGQSPQLLIYTLSYRASGVPDRFSGSGSGTDFTLKISRVEAEDVGIYYCMQRIEFPLTFGGGTKVEIK

SEQ ID NO: 66 - DNA, 339 bp, synthetic construct
  misc_feature 1..339: note = Description of Artificial Sequence: Synthetic polynucleotide
  source 1..339: mol_type = other DNA
gatattgtgatgactcagtctccactctccctgcccgtcacccctggagagccggcctccatctcctgcaggtctagtcagagcctcttggatagtgatgatggaaacacctatttggactggttcctgcagaagccagggcagtctccacagctcctgatctatacgctttcctatcgggcctctggagtcccagacaggttcagtggcagtgggtcaggcacggatttcacactgaaaatcagcagggtggaggctgaggatgttggaatttattactgcatgcaacgtatagagtttccgctcactttcggcggagggaccaaggtggaaatcaaa

SEQ ID NO: 67 - protein, 121 aa, synthetic construct
  REGION 1..121: note = Description of Artificial Sequence: Synthetic polypeptide
QVQLQQSGPGLVRPSETLALTCSVSGVSISNYYWSWIRQPAGRGLEWIGRISPSGRTNYNTSLKSRGTMSLDASKNQFSLKVNSVTAADTAVYYCARGEYSGTYSYSFDIWGQGTMVTVSS

SEQ ID NO: 68 - DNA, 363 bp, synthetic construct
  misc_feature 1..363: note = Description of Artificial Sequence: Synthetic polynucleotide
  source 1..363: mol_type = other DNA
caagttcagcttcaacaatctggtccaggtctcgtaagaccatcagaaacattggctcttacatgctctgttagtggtgtgtcaatcagtaactattactggtcctggatccgccaacctgctggccgtgggctcgaatggatcggacgaatctcacctagcggtaggacaaattacaacacttcccttaaatcacgagggacaatgagcctcgacgcatcaaagaaccagttcagccttaaagtaaactccgttaccgcagcagatactgcagtctactattgtgccaggggtgaatattcaggaacatattcctattcttttgacatttggggccagggaaccatggtaacagtgagttca

SEQ ID NO: 69 - protein, 113 aa, synthetic construct
  REGION 1..113: note = Description of Artificial Sequence: Synthetic polypeptide
DIVMTQTPLSLPVTPGEPASISCRSSQSLFDSDDGNTYLDWFLQKPGQSPQLLIQTLSYRASGVPDRFSGSGSGTDFTLKISRVEADDVGVYYCMQRMEFPLTFGGGTKLEIK

SEQ ID NO: 70 - DNA, 339 bp, synthetic construct
  misc_feature 1..339: note = Description of Artificial Sequence: Synthetic polynucleotide
  source 1..339: mol_type = other DNA
gatattgtgatgactcagactccactctctctgcccgtcacccctggagaaccggcctccatctcctgcaggtctagtcagagcctctttgatagtgatgatggaaacacctatttggactggttcctgcagaagccagggcagtctccacagctcctaatccaaacgctttcctatcgggcctctggagtcccagacaggttcagtggcagtgggtcaggcaccgatttcacactgaaaatcagcagggtggaggctgatgatgttggagtttattactgcatgcaacgtatggagtttccgctcactttcggcggagggaccaagctggagatcaaa

SEQ ID NO: 71 - protein, 121 aa, synthetic construct
  REGION 1..121: note = Description of Artificial Sequence: Synthetic polypeptide
QVQLQESGPGLVKPSETLSLTCSVSGGSISNYYWSWIRQPAGKGLEWIGRIFYSGKTNYNSSLKSRVTMSADTSKNQFSLKLSSVTAADTAVYYCARGEYSGEYSYSFDIWGQGTTVTVSS

SEQ ID NO: 72 - DNA, 363 bp, synthetic construct
  misc_feature 1..363: note = Description of Artificial Sequence: Synthetic polynucleotide
  source 1..363: mol_type = other DNA
caggtacaacttcaggagagcggcccaggtttggttaaaccaagtgaaaccttgtcacttacctgttccgtgtcaggtgggtcaataagcaattactactggtcctggattagacaacctgctggaaaggggcttgaatggatcgggaggatattctactcagggaagacaaactacaatagtagcctcaagtccagggtgaccatgtccgctgatacttccaagaatcaatttagccttaaattgtcctccgttacagccgctgataccgcagtgtactactgtgcaagaggtgagtacagtggcgaatactcatattcctttgacatctggggtcagggcactactgtgactgtttcatct

SEQ ID NO: 73 - protein, 113 aa, synthetic construct
  REGION 1..113: note = Description of Artificial Sequence: Synthetic polypeptide
EIVMTQSPLSLPVTPGEPASISCRSSQSLLDSDDGNTYVDWFLQKPGQSPQLLIYTLSYRASGVPDRFSGSGSDTDFTLKISRVEAEDVGIYYCMQRMEFPLTFGGGTKVEIK

SEQ ID NO: 74 - DNA, 339 bp, synthetic construct
  misc_feature 1..339: note = Description of Artificial Sequence: Synthetic polynucleotide
  source 1..339: mol_type = other DNA
gaaatagtgatgacgcagtctccactctccctgcccgtcacccctggagagccggcctccatttcctgccggtctagtcagagcctcttggatagtgatgatggaaacacctatgtggactggttcctgcagaagccagggcagtctccacaactcctgatctatacgctttcctatcgggcctctggagtcccagacaggttcagtggcagtgggtcagacactgatttcacactgaaaatcagcagggtggaggctgaagatgttggaatttattactgcatgcaacgtatggagtttccgctcactttcggcggagggaccaaggtggagatcaaa

SEQ ID NO: 75 - protein, 12 aa, synthetic construct
  REGION 1..12: note = Description of Artificial Sequence: Synthetic peptide
PQVHYDYAGFPY

SEQ ID NO: 76 - protein, 10 aa, synthetic construct
  REGION 1..10: note = Description of Artificial Sequence: Synthetic peptide
GYTFTRSTMH

SEQ ID NO: 77 - protein, 10 aa, synthetic construct
  REGION 1..10: note = Description of Artificial Sequence: Synthetic peptide
YINPSSAYTN

SEQ ID NO: 78 - protein, 12 aa, synthetic construct
  REGION 1..12: note = Description of Artificial Sequence: Synthetic peptide
PQVHYDYNGFPY

SEQ ID NO: 79 - protein, 10 aa, synthetic construct
  REGION 1..10: note = Description of Artificial Sequence: Synthetic peptide
SASSSVSYMN

SEQ ID NO: 80 - protein, 7 aa, synthetic construct
  REGION 1..7: note = Description of Artificial Sequence: Synthetic peptide
DSSKLAS

SEQ ID NO: 81 - protein, 9 aa, synthetic construct
  REGION 1..9: note = Description of Artificial Sequence: Synthetic peptide
QQWSRNPPT

SEQ ID NO: 82 - protein, 12 aa, synthetic construct
  REGION 1..12: note = Description of Artificial Sequence: Synthetic peptide
PQVHYDYGGFPY

SEQ ID NO: 83 - protein, 10 aa, synthetic construct
  REGION 1..10: note = Description of Artificial Sequence: Synthetic peptide
GFTFSRYNMN

SEQ ID NO: 84 - protein, 10 aa, synthetic construct
  REGION 1..10: note = Description of Artificial Sequence: Synthetic peptide
SISTSSNYIY

SEQ ID NO: 85 - protein, 7 aa, synthetic construct
  REGION 1..7: note = Description of Artificial Sequence: Synthetic peptide
GWGPFDY

SEQ ID NO: 86 - protein, 11 aa, synthetic construct
  REGION 1..11: note = Description of Artificial Sequence: Synthetic peptide
RARQSIGTAIH

SEQ ID NO: 87 - protein, 7 aa, synthetic construct
  REGION 1..7: note = Description of Artificial Sequence: Synthetic peptide
YASESIS

SEQ ID NO: 88 - protein, 9 aa, synthetic construct
  REGION 1..9: note = Description of Artificial Sequence: Synthetic peptide
QQSGSWPYT

SEQ ID NO: 89 - protein, 10 aa, synthetic construct
  REGION 1..10: note = Description of Artificial Sequence: Synthetic peptide
GFTFSSYAMS

SEQ ID NO: 90 - protein, 10 aa, synthetic construct
  REGION 1..10: note = Description of Artificial Sequence: Synthetic peptide
AISGSGGSTY

SEQ ID NO: 91 - protein, 10 aa, synthetic construct
  REGION 1..10: note = Description of Artificial Sequence: Synthetic peptide
YDGIYGELDF

SEQ ID NO: 92 - protein, 11 aa, synthetic construct
  REGION 1..11: note = Description of Artificial Sequence: Synthetic peptide
RASQSISSYLN

SEQ ID NO: 93 - protein, 7 aa, synthetic construct
  REGION 1..7: note = Description of Artificial Sequence: Synthetic peptide
AASSLQS

SEQ ID NO: 94 - protein, 9 aa, synthetic construct
  REGION 1..9: note = Description of Artificial Sequence: Synthetic peptide
QQSYSTPLT

SEQ ID NO: 95 - protein, 12 aa, synthetic construct
  REGION 1..12: note = Description of Artificial Sequence: Synthetic peptide
VYYGSNYWYFDV

SEQ ID NO: 96 - protein, 10 aa, synthetic construct
  REGION 1..10: note = Description of Artificial Sequence: Synthetic peptide
RASSSVSYMH

SEQ ID NO: 97 - protein, 121 aa, synthetic construct
  REGION 1..121: note = Description of Artificial Sequence: Synthetic polypeptide
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYNGFPYWGQGTLVTVSS

SEQ ID NO: 98 - DNA, 363 bp, synthetic construct
  misc_feature 1..363: note = Description of Artificial Sequence: Synthetic polynucleotide
  source 1..363: mol_type = other DNA
caggttcagctggttcagtctggcgccgaagtgaagaaacctggctcctccgtcaaggtgtcctgcaaggcttccggctacacctttaccagatccaccatgcactgggtcaaacaggctccaggacaaggcttggagtggatcggctacatcaaccccagctccgcctacaccaactacaaccagaaattccagggcagagtcaccctcaccgccgacaagtctacctccaccgcctacatggaactgtccagcctgagatctgaggacaccgccgtgtactactgcgccagccctcaggtgcactacgactacaacggcttcccttattggggccagggcaccctggttaccgtttcttct

SEQ ID NO: 99 - protein, 106 aa, synthetic construct
  REGION 1..106: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIK

SEQ ID NO: 100 - DNA, 318 bp, synthetic construct
  misc_feature 1..318: note = Description of Artificial Sequence: Synthetic polynucleotide
  source 1..318: mol_type = other DNA
gagatcgtgctgacccagtctcctgccacactgagtgcttctccaggcgagagagtgaccctgtcctgctccgcttcctcctccgtgtcctacatgaactggtatcagcagaagcccggccaggctcctcggagatggatctacgactcttccaagctggcctctggtgtgccagccagattttctggctctggctccggcagagactataccctgaccatctccagcctggaacctgaggacttcgccgtgtactactgccagcagtggtctaggaaccctcctacctttggcggaggcaccaaggtggaaatcaag

SEQ ID NO: 101 - protein, 121 aa, synthetic construct
  REGION 1..121: note = Description of Artificial Sequence: Synthetic polypeptide
QVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSS

SEQ ID NO: 102 - DNA, 363 bp, synthetic construct
  misc_feature 1..363: note = Description of Artificial Sequence: Synthetic polynucleotide
  source 1..363: mol_type = other DNA
caggttcaactggttcagtctggcgccgaagtgaagaaacctggctcctccgtcaaggtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggtccactacgactacgccggctttccttattggggccagggcacactggtcaccgtttcttct

SEQ ID NO: 103 - protein, 121 aa, synthetic construct
  REGION 1..121: note = Description of Artificial Sequence: Synthetic polypeptide
QVQLVQSGAEVKKPGSSVKVSCKASGYTFTRSTMHWVRQAPGQGLEWMGYINPSSAYTNYAQKFQGRVTLTADKSTSTAYMELSSLRSEDTAVYYCASPQVHYDYNGFPYWGQGTLVTVSS

SEQ ID NO: 104 - DNA, 363 bp, synthetic construct
  misc_feature 1..363: note = Description of Artificial Sequence: Synthetic polynucleotide
  source 1..363: mol_type = other DNA
caggttcagctggttcagtctggcgccgaagtgaagaaacctggctcctccgtcaaggtgtcctgcaaggcttccggctacacctttaccagatccaccatgcactgggtccgacaggctccaggccaaggcttggagtggatgggctacatcaaccccagctccgcctacaccaactacgcccagaaattccagggcagagtcaccctcaccgccgacaagtctacctccaccgcctacatggaactgtccagcctgagatctgaggacaccgccgtgtactactgcgccagccctcaggtgcactacgactacaacggcttcccttattggggccagggcaccctggttaccgtttcttct

SEQ ID NO: 105 - protein, 121 aa, synthetic construct
  REGION 1..121: note = Description of Artificial Sequence: Synthetic polypeptide
QVQLVQSGAEVKKPGSSVKVSCKASGYTFTRSTMHWVRQAPGQGLEWMGYINPSSAYTNYAQKFQGRVTLTADKSTSTAYMELSSLRSEDTAVYYCASPQVHYDYGGFPYWGQGTLVTVSS

SEQ ID NO: 106 - DNA, 363 bp, synthetic construct
  misc_feature 1..363: note = Description of Artificial Sequence: Synthetic polynucleotide
  source 1..363: mol_type = other DNA
caggttcaactggttcagtctggcgccgaagtgaagaaacctggctcctccgtgaaagtgtcctgcaaggcttccggctacacttttaccagatccaccatgcactgggtccgacaggctccaggacaaggcttggagtggatgggctacatcaaccccagctccgcctacaccaactacgcccagaaattccagggcagagtgaccctgaccgccgacaagtctacctccaccgcctacatggaactgtccagcctgagatctgaggacaccgccgtgtactactgcgcttctcctcaggtgcactacgactacggcggctttccttattggggccagggcacactggtcaccgtttcttct

SEQ ID NO: 107 - protein, 116 aa, synthetic construct
  REGION 1..116: note = Description of Artificial Sequence: Synthetic polypeptide
EVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSS

SEQ ID NO: 108 - DNA, 348 bp, synthetic construct
  misc_feature 1..348: note = Description of Artificial Sequence: Synthetic polynucleotide
  source 1..348: mol_type = other DNA
gaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctca

SEQ ID NO: 109 - protein, 107 aa, synthetic construct
  REGION 1..107: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIK

SEQ ID NO: 110 - DNA, 321 bp, synthetic construct
  misc_feature 1..321: note = Description of Artificial Sequence: Synthetic polynucleotide
  source 1..321: mol_type = other DNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaag

SEQ ID NO: 111 - protein, 119 aa, synthetic construct
  REGION 1..119: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSS

SEQ ID NO: 112 - DNA, 357 bp, synthetic construct
  misc_feature 1..357: note = Description of Artificial Sequence: Synthetic polynucleotide
  source 1..357: mol_type = other DNA
gaggtgcagttgctggagtccgggggtggactcgtacaacctggaggttctctgcggttgtcctgtgctgccagcggattcacattttcctcttatgccatgtcctgggtacgtcaagcacccggcaaaggacttgagtgggtctccgctatcagtggttcagggggatcaacctactatgctgatagtgttaaggggcgttttaccatctcaagagacaactccaagaacaccctgtacctgcagatgaactcactccgcgccgaggatacagcagtttactactgtgctaagtatgacggcatttacggcgaactggacttttggggacaggggaccttggtcacagtctccagc

SEQ ID NO: 113 - protein, 107 aa, synthetic construct [sequence omitted]

SEQ ID NO: 114 - DNA, 321 bp, synthetic construct
  misc_feature 1..321: note = Description of Artificial Sequence: Synthetic polynucleotide
  source 1..321: mol_type = other DNA
gatattcagatgactcagagcccctcttcactgagtgcctcagtaggggatcgtgtgactatcacctgtcgtgcttcccaaagcatctcctcatatttgaactggtaccagcagaagccaggcaaggcacccaaactgctgatttacgccgccagttctctccagagtggcgttcccagccgtttctcaggttctggatctggtaccgatttcacattgaccatatcatccctccagcctgaggacttcgccacctactattgccaacagtcatattctactccacttacattcggccagggcaccaaggtggaaattaag

SEQ ID NO: 115 - protein, 10 aa, synthetic construct
  REGION 1..10: note = Description of Artificial Sequence: Synthetic peptide
GYTFTSYNMH

SEQ ID NO: 116 - protein, 10 aa, synthetic construct
  REGION 1..10: note = Description of Artificial Sequence: Synthetic peptide
AIYPGNGDTS

SEQ ID NO: 117 - protein, 12 aa, synthetic construct
  REGION 1..12: note = Description of Artificial Sequence: Synthetic peptide
STYYGGDWYFNV

SEQ ID NO: 118 - protein, 10 aa, synthetic construct
  REGION 1..10: note = Description of Artificial Sequence: Synthetic peptide
RASSSVSYIH

SEQ ID NO: 119 - protein, 7 aa, synthetic construct
  REGION 1..7: note = Description of Artificial Sequence: Synthetic peptide
ATSNLAS

SEQ ID NO: 120 - protein, 9 aa, synthetic construct
  REGION 1..9: note = Description of Artificial Sequence: Synthetic peptide
QQWTSNPPT

SEQ ID NO: 121 - protein, 10 aa, synthetic construct
  REGION 1..10: note = Description of Artificial Sequence: Synthetic peptide
GYTFSSYNMH

SEQ ID NO: 122 - protein, 10 aa, synthetic construct
  REGION 1..10: note = Description of Artificial Sequence: Synthetic peptide
AIYPGAGDTS

SEQ ID NO: 123 - protein, 13 aa, synthetic construct
  REGION 1..13: note = Description of Artificial Sequence: Synthetic peptide
SNYYGSSGWYFDV

SEQ ID NO: 124 - protein, 10 aa, synthetic construct
  REGION 1..10: note = Description of Artificial Sequence: Synthetic peptide
RASLSVSSMH

SEQ ID NO: 125 - protein, 9 aa, synthetic construct
  REGION 1..9: note = Description of Artificial Sequence: Synthetic peptide
QQWIFNPPT

SEQ ID NO: 126 - protein, 121 aa, synthetic construct
  REGION 1..121: note = Description of Artificial Sequence: Synthetic polypeptide
QVQLVQSGAEVKKPGSSVKVSCKASGYTFTSYNMHWVRQAPGQGLEWMGAIYPGNGDTSYAQKFQGRVTITADKSTSTAYMELSSLRSEDTAVYYCARSTYYGGDWYFNVWGQGTLVTVSS

SEQ ID NO: 127 - DNA, 363 bp, synthetic construct
  misc_feature 1..363: note = Description of Artificial Sequence: Synthetic polynucleotide
  source 1..363: mol_type = other DNA
caggttcagctggttcagtctggtgccgaagtgaagaaacctggctcctccgtgaaagtgtcctgcaaggcttccggctacacttttaccagctacaacatgcactgggtccgacaggcccctggacaaggattggaatggatgggcgctatctaccccggcaacggcgatacctcttacgcccagaaattccagggcagagtgaccatcaccgccgacaagtctacctccaccgcctacatggaactgtccagcctgagatctgaggacaccgccgtgtactactgcgcccggtctacctattatggcggcgactggtacttcaacgtgtggggccagggaaccctggtcacagtctcttct

SEQ ID NO: 128 - protein, 106 aa, synthetic construct
  REGION 1..106: note = Description of Artificial Sequence: Synthetic polypeptide
AIQLTQSPSSLSASVGDRVTITCRASSSVSYIHWFQQKPGKAPKPLIYATSNLASGVPSRFSGSGSGTDFTLTISSLQPEDFATYYCQQWTSNPPTFGQGTKLEIK

SEQ ID NO: 129 - DNA, 318 bp, synthetic construct
  misc_feature 1..318: note = Description of Artificial Sequence: Synthetic polynucleotide
  source 1..318: mol_type = other DNA
gccattcagctgacccagtctccatcctctctgtccgcctctgtgggcgacagagtgacaattacctgccgggcctcctcctccgtgtcctacatccattggttccagcagaagcccggcaaggcccctaagcctctgatctacgccacctccaatctggcctctggcgtgccctccagattttccggatctggctctggaaccgactttaccctgacaatctccagcctgcagcctgaggacttcgccacctactactgtcagcagtggaccagcaatcctcctacctttggccagggcaccaagctggaaatcaag

SEQ ID NO: 130 - protein, 122 aa, synthetic construct
  REGION 1..122: note = Description of Artificial Sequence: Synthetic polypeptide
QVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSS

SEQ ID NO: 131 - DNA, 366 bp, synthetic construct
  misc_feature 1..366: note = Description of Artificial Sequence: Synthetic polynucleotide
  source 1..366: mol_type = other DNA
caggttcaactggttcagtctggcgccgaagtgaagaaacctggctcctccgtgaaggtgtcctgcaaggcttccggctacaccttctccagctacaacatgcactgggtccgacaggcccctggacaaggattggaatggatgggcgctatctaccctggcgctggcgatacctcttacgcccagaaattccagggcagagtgaccatcaccgccgacgagtctacctccaccgcctacatggaactgtccagcctgagatctgaggacaccgccgtgtactactgcgcccggtctaattactacggctccagcggctggtacttcgacgtgtggggaaagggcaccaccgtgacagtctcttct

SEQ ID NO: 132 - protein, 106 aa, synthetic construct
  REGION 1..106: note = Description of Artificial Sequence: Synthetic polypeptide
EIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIK

SEQ ID NO: 133 - DNA, 318 bp, synthetic construct
  misc_feature 1..318: note = Description of Artificial Sequence: Synthetic polynucleotide
  source 1..318: mol_type = other DNA
gagatcgtgctgacccagtctccagccacactgtcactgtctccaggcgagagagctaccctgtcctgtagagcctctctgtccgtgtcctccatgcactggtatcagcagaagcctggacaggcccctcggctgctgatctacgctacctctaatctggccagcggtatccccgccagattttctggttctggctctggcaccgactttaccctgaccatctccagcctggaacctgaggacttcgccgtgtactactgccagcagtggatcttcaaccctcctacctttggcggaggcaccaaggtggaaatcaag

SEQ ID NO: 134 - protein, 121 aa, synthetic construct
  REGION 1..121: note = Description of Artificial Sequence: Synthetic polypeptide
QAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVYYGSNYWYFDVWGTGTTVTVSS

SEQ ID NO: 135 - DNA, 363 bp, synthetic construct
  misc_feature 1..363: note = Description of Artificial Sequence: Synthetic polynucleotide
  source 1..363: mol_type = other DNA
caagcatatctgcaacagagcggagctgagctggttcggcctggcgcctctgtaaaaatgagttgcaaggccagtggttatacattcacatcatataatatgcactgggtaaagcaaactccccgacaggggcttgaatggattggcgcaatctatcccggcaatggggatacatcctacaatcagaaattcaagggcaaggcaacactgaccgttgacaaatcctcatcaacagcctacatgcagctcagttccctcactagcgaagattctgctgtgtatttctgtgcaagggtgtattatggttctaattactggtatttcgatgtttggggaaccggaactaccgtaactgtttctagc

SEQ ID NO: 136 - protein, 106 aa, synthetic construct
  REGION 1..106: note = Description of Artificial Sequence: Synthetic polypeptide
QIVLSQSPAILSASPGEKVTMTCRASSSVSYMHWYQQKPGSSPQVWIYATSNLASGVPVRFSGSGSGTSYSLTISRVEAEDTATYYCQQWIFNPPTFGSGTKLEIR

SEQ ID NO: 137 - DNA, 318 bp, synthetic construct
  misc_feature 1..318: note = Description of Artificial Sequence: Synthetic polynucleotide
  source 1..318: mol_type = other DNA
caaatagtcctttcacagtccccagctattctttcagcctctcccggtgaaaaggttacaatgacctgccgggcaagctccagtgtctcatatatgcactggtaccaacaaaaacctggcagtagtcctcaggtgtggatctacgctacaagcaatctcgcttccggggttcccgtgaggtttagcggaagcgggtctggaactagttattccttgacaattagtcgggttgaagccgaggacaccgccacttactattgccaacagtggatattcaatccacccaccttcggttcaggtaccaagctcgaaatccgt

SEQ ID NO: 138 - protein, 122 aa, synthetic construct
  REGION 1..122: note = Description of Artificial Sequence: Synthetic polypeptide
QAYLQQSGAELVRPGASVKMSCKTSGYTFSSYNMHWVKQTPRQALEWIGAIYPGNGDTSYNQKFKGKATLTVDKSSSTAYMQLSSLTSEDSAVYFCTRSNYYGSSGWYFDVWGTGTTVTVSS

SEQ ID NO: 139 - DNA, 366 bp, synthetic construct
  misc_feature 1..366: note = Description of Artificial Sequence: Synthetic polynucleotide
  source 1..366: mol_type = other DNA
caagcctatcttcaacaatctggggctgagcttgtccggccaggagcctccgtcaaaatgagctgcaaaacctcaggttatacttttagtagctataacatgcattgggtaaaacaaaccccccgacaagcattggagtggataggggccatataccccggcaatggagacacaagttacaaccagaagtttaaaggcaaagctacactcacagttgacaaatcctcaagtactgcttatatgcaactctcctctctcacttccgaagacagtgccgtatatttttgcactcggtccaattactatggatctagtggctggtactttgacgtttggggcactgggacaactgttacagtgtccagc

SEQ ID NO: 140 - protein, 106 aa, synthetic construct
  REGION 1..106: note = Description of Artificial Sequence: Synthetic polypeptide
QIVLSQSPAILSASPGEKVTMTCRASLSVSSMHWYQQKPGSSPKPWIYATSNLASGVPARFSGSGSGTSYSLTISRVEAEDAATYYCQQWIFNPPTFGGGTKLEIK

SEQ ID NO: 141 - DNA, 318 bp, synthetic construct
  misc_feature 1..318: note = Description of Artificial Sequence: Synthetic polynucleotide
  source 1..318: mol_type = other DNA
cagattgtcctgagccaatccccagcaattctgagtgctagccctggagagaaggtaacaatgacttgtcgggcatcccttagcgtctcatccatgcattggtatcaacaaaagccaggttcatctccaaaaccctggatttacgctacatctaacctggcatctggggtgcctgccagatttagtggatctggttccggcacatcatattcccttacaatcagccgagtggaagccgaggatgctgcaacctattactgtcaacaatggatatttaaccctcccacctttgggggtgggactaaactcgaaatcaag

SEQ ID NO: 142 - protein, 746 aa, synthetic construct
  REGION 1..746: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGSEGKSSGSGSESKSTGGSAIQLTQSPSSLSASVGDRVTITCRASSSVSYIHWFQQKPGKAPKPLIYATSNLASGVPSRFSGSGSGTDFTLTISSLQPEDFATYYCQQWTSNPPTFGQGTKLEIKGGSEGKSSGSGSESKSTGGSQVQLVQSGAEVKKPGSSVKVSCKASGYTFTSYNMHWVRQAPGQGLEWMGAIYPGNGDTSYAQKFQGRVTITADKSTSTAYMELSSLRSEDTAVYYCARSTYYGGDWYFNVWGQGTLVTVSS

SEQ ID NO: 143 - DNA, 2238 bp, synthetic construct
  misc_feature 1..2238: note = Description of Artificial Sequence: Synthetic polynucleotide
  source 1..2238: mol_type = other DNA
gagatcgtgctgacccagtctcctgccacactgagtgcttctccaggcgagagagtgaccctgtcctgctccgcttcctcctccgtgtcctacatgaactggtatcagcagaagcccggccaggctcctcggagatggatctacgactcttccaagctggcctctggtgtgccagccagattttctggctctggctccggcagagactataccctgaccatctccagcctggaacctgaggacttcgccgtgtactactgccagcagtggtctaggaaccctcctacctttggcggaggcaccaaggtggaaatcaagggcggatctgagggaaagtccagcggctccggcagcgaaagcaagtccaccggcggaagccaggttcaactggttcagtctggcgccgaagtgaagaaacctggctcctccgtcaaggtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggtccactacgactacgccggctttccttattggggccagggcacactggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaaggagggagcgagggaaagtccagcggaagcggctctgagtccaaatccaccggagggagcgccattcagctgacccagtctccatcctctctgtccgcctctgtgggcgacagagtgacaattacctgccgggcctcctcctccgtgtcctacatccattggttccagcagaagcccggcaaggcccctaagcctctgatctacgccacctccaatctggcctctggcgtgccctccagattttccggatctggctctggaaccgactttaccctgacaatctccagcctgcagcctgaggacttcgccacctactactgtcagcagtggaccagcaatcctcctacctttggccagggcaccaagctggaaatcaagggcggctccgagggcaagagcagcggcagcggcagcgagagcaagagcaccggcggcagccaggttcagctggttcagtctggtgccgaagtgaagaaacctggctcctccgtgaaagtgtcctgcaaggcttccggctacacttttaccagctacaacatgcactgggtccgacaggcccctggacaaggattggaatggatgggcgctatctaccccggcaacggcgatacctcttacgcccagaaattccagggcagagtgaccatcaccgccgacaagtctacctccaccgcctacatggaactgtccagcctgagatctgaggacaccgccgtgtactactgcgcccggtctacctattatggcggcgactggtacttcaacgtgtggggccagggaaccctggtcacagtctcttct

SEQ ID NO: 144 - protein, 746 aa, synthetic construct
  REGION 1..746: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVRQAPGQGLEWMGYINPSSAYTNYAQKFQGRVTLTADKSTSTAYMELSSLRSEDTAVYYCASPQVHYDYGGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGSEGKSSGSGSESKSTGGSAIQLTQSPSSLSASVGDRVTITCRASSSVSYIHWFQQKPGKAPKPLIYATSNLASGVPSRFSGSGSGTDFTLTISSLQPEDFATYYCQQWTSNPPTFGQGTKLEIKGGSEGKSSGSGSESKSTGGSQVQLVQSGAEVKKPGSSVKVSCKASGYTFTSYNMHWVRQAPGQGLEWMGAIYPGNGDTSYAQKFQGRVTITADKSTSTAYMELSSLRSEDTAVYYCARSTYYGGDWYFNVWGQGTLVTVSS

SEQ ID NO: 145 - DNA, 2238 bp, synthetic construct
  misc_feature 1..2238: note = Description of Artificial Sequence: Synthetic polynucleotide
  source 1..2238: mol_type = other DNA
gagatcgtgctgacccagtctcctgccacactgagtgcttctccaggcgagagagtgaccctgtcctgctccgcttcctcctccgtgtcctacatgaactggtatcagcagaagcccggccaggctcctcggagatggatctacgactcttccaagctggcctctggtgtgccagccagattttctggctctggctccggcagagactataccctgaccatctccagcctggaacctgaggacttcgccgtgtactactgccagcagtggtctaggaaccctcctacctttggcggaggcaccaaggtggaaatcaagggcggatctgagggaaagtccagcggctccggcagcgaaagcaagtccaccggcggaagccaggttcaactggttcagtctggcgccgaagtgaagaaacctggctcctccgtgaaagtgtcctgcaaggcttccggctacacttttaccagatccaccatgcactgggtccgacaggctccaggacaaggcttggagtggatgggctacatcaaccccagctccgcctacaccaactacgcccagaaattccagggcagagtgaccctgaccgccgacaagtctacctccaccgcctacatggaactgtccagcctgagatctgaggacaccgccgtgtactactgcgcttctcctcaggtgcactacgactacggcggctttccttattggggccagggcacactggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaaggagggagcgagggaaagtccagcggaagcggctctgagtccaaatccaccggagggagcgccattcagctgacccagtctccatcctctctgtccgcctctgtgggcgacagagtgacaattacctgccgggcctcctcctccgtgtcctacatccattggttccagcagaagcccggcaaggcccctaagcctctgatctacgccacctccaatctggcctctggcgtgccctccagattttccggatctggctctggaaccgactttaccctgacaatctccagcctgcagcctgaggacttcgccacctactactgtcagcagtggaccagcaatcctcctacctttggccagggcaccaagctggaaatcaagggcggctccgagggcaagagcagcggcagcggcagcgagagcaagagcaccggcggcagccaggttcagctggttcagtctggtgccgaagtgaagaaacctggctcctccgtgaaagtgtcctgcaaggcttccggctacacttttaccagctacaacatgcactgggtccgacaggcccctggacaaggattggaatggatgggcgctatctaccccggcaacggcgatacctcttacgcccagaaattccagggcagagtgaccatcaccgccgacaagtctacctccaccgcctacatggaactgtccagcctgagatctgaggacaccgccgtgtactactgcgcccggtctacctattatggcggcgactggtacttcaacgtgtggggccagggaaccctggtcacagtctcttct

SEQ ID NO: 146 - protein, 735 aa, synthetic construct
  REGION 1..735: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGGSEGKSSGSGSESKSTGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKYDGIYGELDFWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSAIQLTQSPSSLSASVGDRVTITCRASSSVSYIHWFQQKPGKAPKPLIYATSNLASGVPSRFSGSGSGTDFTLTISSLQPEDFATYYCQQWTSNPPTFGQGTKLEIKGGSEGKSSGSGSESKSTGGSQVQLVQSGAEVKKPGSSVKVSCKASGYTFTSYNMHWVRQAPGQGLEWMGAIYPGNGDTSYAQKFQGRVTITADKSTSTAYMELSSLRSEDTAVYYCARSTYYGGDWYFNVWGQGTLVTVSS

SEQ ID NO: 147 - DNA, 2205 bp, synthetic construct
  misc_feature 1..2205: note = Description of Artificial Sequence: Synthetic polynucleotide
  source 1..2205: mol_type = other DNA
gatattcagatgactcagagcccctcttcactgagtgcctcagtaggggatcgtgtgactatcacctgtcgtgcttcccaaagcatctcctcatatttgaactggtaccagcagaagccaggcaaggcacccaaactgctgatttacgccgccagttctctccagagtggcgttcccagccgtttctcaggttctggatctggtaccgatttcacattgaccatatcatccctccagcctgaggacttcgccacctactattgccaacagtcatattctactccacttacattcggccagggcaccaaggtggaaattaagggcggctccgagggcaagagcagcggcagcggcagcgagagcaagagcaccggcggcagcgaggtgcagttgctggagtccgggggtggactcgtacaacctggaggttctctgcggttgtcctgtgctgccagcggattcacattttcctcttatgccatgtcctgggtacgtcaagcacccggcaaaggacttgagtgggtctccgctatcagtggttcagggggatcaacctactatgctgatagtgttaaggggcgttttaccatctcaagagacaactccaagaacaccctgtacctgcagatgaactcactccgcgccgaggatacagcagtttactactgtgctaagtatgacggcatttacggcgaactggacttttggggacaggggaccttggtcacagtctccagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctgcaatccaactaactcaaagtccaagtagtctgtctgcttccgtgggcgacagagtgacaatcacctgtagagcctccagcagcgtctcctacatccactggttccagcaaaaacctggcaaggcccctaagcctctgatctacgccacctccaacctggcctctggcgtgccctctcggttctccggctctggctccggaaccgacttcaccctgaccatctccagcctgcagcctgaggattttgctacctactactgccagcagtggacctctaaccctccaacattcggccagggcaccaagctggaaatcaagggcggctccgagggcaagagcagcggcagcggcagcgagagcaagagcaccggcggcagccaagtgcaattagtgcaaagtggtgcagaagtcaagaagcctggaagctccgtgaaagtgtcctgcaaggcctctggctacacctttacctcctacaacatgcactgggtgcggcaggctcctggccagggcctggagtggatgggcgctatctaccccggcaacggcgatacctcttacgcccagaagttccagggcagagtgaccatcaccgccgacaagtccacatctacagcctacatggaactgtcctccctgcggtccgaggacaccgctgtgtactattgtgccagatctacctactacggcggcgactggtacttcaacgtgtggggccaaggaaccctggtgaccgtgtctagc

SEQ ID NO: 148 - protein, 732 aa, synthetic construct
  REGION 1..732: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSAIQLTQSPSSLSASVGDRVTITCRASSSVSYIHWFQQKPGKAPKPLIYATSNLASGVPSRFSGSGSGTDFTLTISSLQPEDFATYYCQQWTSNPPTFGQGTKLEIKGGSEGKSSGSGSESKSTGGSQVQLVQSGAEVKKPGSSVKVSCKASGYTFTSYNMHWVRQAPGQGLEWMGAIYPGNGDTSYAQKFQGRVTITADKSTSTAYMELSSLRSEDTAVYYCARSTYYGGDWYFNVWGQGTLVTVSS

SEQ ID NO: 149 - DNA, 2196 bp, synthetic construct
  misc_feature 1..2196: note = Description of Artificial Sequence: Synthetic polynucleotide
  source 1..2196: mol_type = other DNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagggcggctccgagggcaagagcagcggcagcggcagcgagagcaagagcaccggcggcagcgaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctgcaatccaactaactcaaagtccaagtagtctgtctgcttccgtgggcgacagagtgacaatcacctgtagagcctccagcagcgtctcctacatccactggttccagcaaaaacctggcaaggcccctaagcctctgatctacgccacctccaacctggcctctggcgtgccctctcggttctccggctctggctccggaaccgacttcaccctgaccatctccagcctgcagcctgaggattttgctacctactactgccagcagtggacctctaaccctccaacattcggccagggcaccaagctggaaatcaagggcggctccgagggcaagagcagcggcagcggcagcgagagcaagagcaccggcggcagccaagtgcaattagtgcaaagtggtgcagaagtcaagaagcctggaagctccgtgaaagtgtcctgcaaggcctctggctacacctttacctcctacaacatgcactgggtgcggcaggctcctggccagggcctggagtggatgggcgctatctaccccggcaacggcgatacctcttacgcccagaagttccagggcagagtgaccatcaccgccgacaagtccacatctacagcctacatggaactgtcctccctgcggtccgaggacaccgctgtgtactattgtgccagatctacctactacggcggcgactggtacttcaacgtgtggggccaaggaaccctggtgaccgtgtctagc

SEQ ID NO: 150 - protein, 736 aa, synthetic construct
  REGION 1..736: note = Description of Artificial Sequence: Synthetic polypeptide
QVQLVQSGAEVKKPGSSVKVSCKASGYTFTRSTMHWVRQAPGQGLEWMGYINPSSAYTNYAQKFQGRVTLTADKSTSTAYMELSSLRSEDTAVYYCASPQVHYDYGGFPYWGQGTLVTVSSGGSEGKSSGSGSESKSTGGSEIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSAIQLTQSPSSLSASVGDRVTITCRASSSVSYIHWFQQKPGKAPKPLIYATSNLASGVPSRFSGSGSGTDFTLTISSLQPEDFATYYCQQWTSNPPTFGQGTKLEIKGGSEGKSSGSGSESKSTGGSQVQLVQSGAEVKKPGSSVKVSCKASGYTFTSYNMHWVRQAPGQGLEWMGAIYPGNGDTSYAQKFQGRVTITADKSTSTAYMELSSLRSEDTAVYYCARSTYYGGDWYFNVWGQGTLVTVSS

SEQ ID NO: 151 - DNA, 2208 bp, synthetic construct
  misc_feature 1..2208: note = Description of Artificial Sequence: Synthetic polynucleotide
  source 1..2208: mol_type = other DNA
caggtgcagctggtgcagagcggcgccgaggtgaagaagcctggcagcagcgtgaaggtgagctgtaaggccagcggctacactttcactaggagcactatgcactgggtgaggcaggcccctggccagggcctggagtggatgggctacatcaatcctagcagcgcctacactaattacgcccagaagttccagggcagggtgactctgactgccgataagagcactagcactgcctacatggagctgagcagcctgaggagcgaggatactgccgtgtactactgtgccagccctcaggtgcactacgattacggcggcttcccttactggggccagggcactctggtgactgtgagcagcggcggctccgagggcaagagcagcggcagcggcagcgagagcaagagcaccggcggcagcgagatcgtgctgactcagagccctgccactctgagcgccagccctggcgagagggtgactctgagctgtagcgccagcagcagcgtgagctacatgaattggtaccagcagaagcctggccaggcccctaggaggtggatctacgatagcagcaagctggccagcggcgtgcctgccaggttcagcggcagcggcagcggcagggattacactctgactatcagcagcctggagcctgaggatttcgccgtgtactactgtcagcagtggagcaggaatcctcctactttcggcggcggcactaaggtggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctgcaatccaactaactcaaagtccaagtagtctgtctgcttccgtgggcgacagagtgacaatcacctgtagagcctccagcagcgtctcctacatccactggttccagcaaaaacctggcaaggcccctaagcctctgatctacgccacctccaacctggcctctggcgtgccctctcggttctccggctctggctccggaaccgacttcaccctgaccatctccagcctgcagcctgaggattttgctacctactactgccagcagtggacctctaaccctccaacattcggccagggcaccaagctggaaatcaagggcggctccgagggcaagagcagcggcagcggcagcgagagcaagagcaccggcggcagccaagtgcaattagtgcaaagtggtgcagaagtcaagaagcctggaagctccgtgaaagtgtcctgcaaggcctctggctacacctttacctcctacaacatgcactgggtgcggcaggctcctggccagggcctggagtggatgggcgctatctaccccggcaacggcgatacctcttacgcccagaagttccagggcagagtgaccatcaccgccgacaagtccacatctacagcctacatggaactgtcctccctgcggtccgaggacaccgctgtgtactattgtgccagatctacctactacggcggcgactggtacttcaacgtgtggggccaaggaaccctggtgaccgtgtctagc

SEQ ID NO: 152 - protein, 732 aa, synthetic construct
  REGION 1..732: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSQIVLSQSPAILSASPGEKVTMTCRASSSVSYMHWYQQKPGSSPQVWIYATSNLASGVPVRFSGSGSGTSYSLTISRVEAEDTATYYCQQWIFNPPTFGSGTKLEIRGGSEGKSSGSGSESKSTGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVYYGSNYWYFDVWGTGTTVTVSS

SEQ ID NO: 153 - DNA, 2196 bp, synthetic construct
  misc_feature 1..2196: note = Description of Artificial Sequence: Synthetic polynucleotide
  source 1..2196: mol_type = other DNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagggcggctccgagggcaagagcagcggcagcggcagcgagagcaagagcaccggcggcagcgaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctcaaatagtcctttcacagtccccagctattctttcagcctctcccggtgaaaaggttacaatgacctgccgggcaagctccagtgtctcatatatgcactggtaccaacaaaaacctggcagtagtcctcaggtgtggatctacgctacaagcaatctcgcttccggggttcccgtgaggtttagcggaagcgggtctggaactagttattccttgacaattagtcgggttgaagccgaggacaccgccacttactattgccaacagtggatattcaatccacccaccttcggttcaggtaccaagctcgaaatccgtggcggctccgagggcaagagcagcggcagcggcagcgagagcaagagcaccggcggcagccaagcatatctgcaacagagcggagctgagctggttcggcctggcgcctctgtaaaaatgagttgcaaggccagtggttatacattcacatcatataatatgcactgggtaaagcaaactccccgacaggggcttgaatggattggcgcaatctatcccggcaatggggatacatcctacaatcagaaattcaagggcaaggcaacactgaccgttgacaaatcctcatcaacagcctacatgcagctcagttccctcactagcgaagattctgctgtgtatttctgtgcaagggtgtattatggttctaattactggtatttcgatgtttggggaaccggaactaccgtaactgtttctagc

SEQ ID NO: 154 - protein, 733 aa, synthetic construct
  REGION 1..733: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSQIVLSQSPAILSASPGEKVTMTCRASLSVSSMHWYQQKPGSSPKPWIYATSNLASGVPARFSGSGSGTSYSLTISRVEAEDAATYYCQQWIFNPPTFGGGTKLEIKGGSEGKSSGSGSESKSTGGSQAYLQQSGAELVRPGASVKMSCKTSGYTFSSYNMHWVKQTPRQALEWIGAIYPGNGDTSYNQKFKGKATLTVDKSSSTAYMQLSSLTSEDSAVYFCTRSNYYGSSGWYFDVWGTGTTVTVSS

SEQ ID NO: 155 - DNA, 2199 bp, synthetic construct
  misc_feature 1..2199: note = Description of Artificial Sequence: Synthetic polynucleotide
  source 1..2199: mol_type = other DNA
gacatacaaatgacacaatcaccctcttctctttctgcaagcgttggcgaccgtgtcactatcacttgtcgagcccgccagtccataggtactgccattcactggtatcaacagaagcctggcaaggctcccaaactcctgattaagtatgccagcgagagcatttccggcgtaccttcaagattttccggctccggtagtgggacagatttcactctcactatatctagcctccaaccagaagatttcgccacttactactgtcaacaatcaggttcatggccttacactttcggccaggggacaaaattggagatcaagggcggctccgagggcaagagcagcggcagcggcagcgagagcaagagcaccggcggcagcgaggtgcaactggtggagtctgggggaggcctggtcaagcctggggggtccctgagactctcctgtgcagcctctggattcaccttcagtagatataacatgaactgggtccgccaggctccagggaaggggctggagtgggtctcatccattagtactagtagtaattacatatactacgcagactcagtgaagggccgattcaccttctccagagacaacgccaagaactcactggatctgcaaatgagcggcctgagagccgaggacacggctatttattactgtacgagaggctgggggccttttgactactggggccagggaaccctggtcaccgtctcctcagagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctcagattgtcctgagccaatccccagcaattctgagtgctagccctggagagaaggtaacaatgacttgtcgggcatcccttagcgtctcatccatgcattggtatcaacaaaagccaggttcatctccaaaaccctggatttacgctacatctaacctggcatctggggtgcctgccagatttagtggatctggttccggcacatcatattcccttacaatcagccgagtggaagccgaggatgctgcaacctattactgtcaacaatggatatttaaccctcccacctttgggggtgggactaaactcgaaatcaagggcggctccgagggcaagagcagcggcagcggcagcgagagcaagagcaccggcggcagccaagcctatcttcaacaatctggggctgagcttgtccggccaggagcctccgtcaaaatgagctgcaaaacctcaggttatacttttagtagctataacatgcattgggtaaaacaaaccccccgacaagcattggagtggataggggccatataccccggcaatggagacacaagttacaaccagaagtttaaaggcaaagctacactcacagttgacaaatcctcaagtactgcttatatgcaactctcctctctcacttccgaagacagtgccgtatatttttgcactcggtccaattactatggatctagtggctggtactttgacgtttggggcactgggacaactgttacagtgtccagc

SEQ ID NO: 156 - protein, 736 aa, synthetic construct
  REGION 1..736: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSQIVLSQSPAILSASPGEKVTMTCRASSSVSYMHWYQQKPGSSPQVWIYATSNLASGVPVRFSGSGSGTSYSLTISRVEAEDTATYYCQQWIFNPPTFGSGTKLEIRGGSEGKSSGSGSESKSTGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVYYGSNYWYFDVWGTGTTVTVSS

SEQ ID NO: 157 - DNA, 2208 bp, synthetic construct
  misc_feature 1..2208: note = Description of Artificial Sequence: Synthetic polynucleotide
  source 1..2208: mol_type = other DNA
gagatcgtgctgactcagagccctgccactctgagcgccagccctggcgagagggtgactctgagctgtagcgccagcagcagcgtgagctacatgaattggtaccagcagaagcctggccaggcccctaggaggtggatctacgatagcagcaagctggccagcggcgtgcctgccaggttcagcggcagcggcagcggcagggattacactctgactatcagcagcctggagcctgaggatttcgccgtgtactactgtcagcagtggagcaggaatcctcctactttcggcggcggcactaaggtggagatcaagggcggctccgagggcaagagcagcggcagcggcagcgagagcaagagcaccggcggcagccaggtgcagctggtgcagagcggcgccgaggtgaagaagcctggcagcagcgtgaaggtgagctgtaaggccagcggctacactttcactaggagcactatgcactgggtgaagcaggcccctggccagggcctggagtggatcggctacatcaatcctagcagcgcctacactaattacaatcagaagttccagggcagggtgactctgactgccgataagagcactagcactgcctacatggagctgagcagcctgaggagcgaggatactgccgtgtactactgtgccagccctcaggtgcactacgattacgccggcttcccttactggggccagggcactctggtgactgtgagcagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctcaaatagtcctttcacagtccccagctattctttcagcctctcccggtgaaaaggttacaatgacctgccgggcaagctccagtgtctcatatatgcactggtaccaacaaaaacctggcagtagtcctcaggtgtggatctacgctacaagcaatctcgcttccggggttcccgtgaggtttagcggaagcgggtctggaactagttattccttgacaattagtcgggttgaagccgaggacaccgccacttactattgccaacagtggatattcaatccacccaccttcggttcaggtaccaagctcgaaatccgtggcggctccgagggcaagagcagcggcagcggcagcgagagcaagagcaccggcggcagccaagcatatctgcaacagagcggagctgagctggttcggcctggcgcctctgtaaaaatgagttgcaaggccagtggttatacattcacatcatataatatgcactgggtaaagcaaactccccgacaggggcttgaatggattggcgcaatctatcccggcaatggggatacatcctacaatcagaaattcaagggcaaggcaacactgaccgttgacaaatcctcatcaacagcctacatgcagctcagttccctcactagcgaagattctgctgtgtatttctgtgcaagggtgtattatggttctaattactggtatttcgatgtttggggaaccggaactaccgtaactgtttctagc

SEQ ID NO: 158 - protein, 737 aa, synthetic construct
  REGION 1..737: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSQIVLSQSPAILSASPGEKVTMTCRASLSVSSMHWYQQKPGSSPKPWIYATSNLASGVPARFSGSGSGTSYSLTISRVEAEDAATYYCQQWIFNPPTFGGGTKLEIKGGSEGKSSGSGSESKSTGGSQAYLQQSGAELVRPGASVKMSCKTSGYTFSSYNMHWVKQTPRQALEWIGAIYPGNGDTSYNQKFKGKATLTVDKSSSTAYMQLSSLTSEDSAVYFCTRSNYYGSSGWYFDVWGTGTTVTVSS

SEQ ID NO: 159 - DNA, 2211 bp, synthetic construct
  misc_feature 1..2211: note = Description of Artificial Sequence: Synthetic polynucleotide
  source 1..2211: mol_type = other DNA
gagatcgtgctgactcagagccctgccactctgagcgccagccctggcgagagggtgactctgagctgtagcgccagcagcagcgtgagctacatgaattggtaccagcagaagcctggccaggcccctaggaggtggatctacgatagcagcaagctggccagcggcgtgcctgccaggttcagcggcagcggcagcggcagggattacactctgactatcagcagcctggagcctgaggatttcgccgtgtactactgtcagcagtggagcaggaatcctcctactttcggcggcggcactaaggtggagatcaagggcggctccgagggcaagagcagcggcagcggcagcgagagcaagagcaccggcggcagccaggtgcagctggtgcagagcggcgccgaggtgaagaagcctggcagcagcgtgaaggtgagctgtaaggccagcggctacactttcactaggagcactatgcactgggtgaagcaggcccctggccagggcctggagtggatcggctacatcaatcctagcagcgcctacactaattacaatcagaagttccagggcagggtgactctgactgccgataagagcactagcactgcctacatggagctgagcagcctgaggagcgaggatactgccgtgtactactgtgccagccctcaggtgcactacgattacgccggcttcccttactggggccagggcactctggtgactgtgagcagcgagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctcagattgtcctgagccaatccccagcaattctgagtgctagccctggagagaaggtaacaatgacttgtcgggcatcccttagcgtctcatccatgcattggtatcaacaaaagccaggttcatctccaaaaccctggatttacgctacatctaacctggcatctggggtgcctgccagatttagtggatctggttccggcacatcatattcccttacaatcagccgagtggaagccgaggatgctgcaacctattactgtcaacaatggatatttaaccctcccacctttgggggtgggactaaactcgaaatcaagggcggctccgagggcaagagcagcggcagcggcagcgagagcaagagcaccggcggcagccaagcctatcttcaacaatctggggctgagcttgtccggccaggagcctccgtcaaaatgagctgcaaaacctcaggttatacttttagtagctataacatgcattgggtaaaacaaaccccccgacaagcattggagtggataggggccatataccccggcaatggagacacaagttacaaccagaagtttaaaggcaaagctacactcacagttgacaaatcctcaagtactgcttatatgcaactctcctctctcacttccgaagacagtgccgtatatttttgcactcggtccaattactatggatctagtggctggtactttgacgtttggggcactgggacaactgttacagtgtccagc

SEQ ID NO: 160 - protein, 736 aa, synthetic construct
  REGION 1..736: note = Description of Artificial Sequence: Synthetic polypeptide
QVQLVQSGAEVKKPGSSVKVSCKASGYTFTRSTMHWVRQAPGQGLEWMGYINPSSAYTNYAQKFQGRVTLTADKSTSTAYMELSSLRSEDTAVYYCASPQVHYDYGGFPYWGQGTLVTVSSGGSEGKSSGSGSESKSTGGSEIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSQIVLSQSPAILSASPGEKVTMTCRASSSVSYMHWYQQKPGSSPQVWIYATSNLASGVPVRFSGSGSGTSYSLTISRVEAEDTATYYCQQWIFNPPTFGSGTKLEIRGGSEGKSSGSGSESKSTGGSQAYLQQSGAELVRPGASVKMSCKASGYTFTSYNMHWVKQTPRQGLEWIGAIYPGNGDTSYNQKFKGKATLTVDKSSSTAYMQLSSLTSEDSAVYFCARVYYGSNYWYFDVWGTGTTVTVSS

SEQ ID NO: 161 - DNA, 2208 bp, synthetic construct
  misc_feature 1..2208: note = Description of Artificial Sequence: Synthetic polynucleotide
  source 1..2208: mol_type = other DNA
caggtgcagctggtgcagagcggcgccgaggtgaagaagcctggcagcagcgtgaaggtgagctgtaaggccagcggctacactttcactaggagcactatgcactgggtgaggcaggcccctggccagggcctggagtggatgggctacatcaatcctagcagcgcctacactaattacgcccagaagttccagggcagggtgactctgactgccgataagagcactagcactgcctacatggagctgagcagcctgaggagcgaggatactgccgtgtactactgtgccagccctcaggtgcactacgattacggcggcttcccttactggggccagggcactctggtgactgtgagcagcggcggctccgagggcaagagcagcggcagcggcagcgagagcaagagcaccggcggcagcgagatcgtgctgactcagagccctgccactctgagcgccagccctggcgagagggtgactctgagctgtagcgccagcagcagcgtgagctacatgaattggtaccagcagaagcctggccaggcccctaggaggtggatctacgatagcagcaagctggccagcggcgtgcctgccaggttcagcggcagcggcagcggcagggattacactctgactatcagcagcctggagcctgaggatttcgccgtgtactactgtcagcagtggagcaggaatcctcctactttcggcggcggcactaaggtggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctcaaatagtcctttcacagtccccagctattctttcagcctctcccggtgaaaaggttacaatgacctgccgggcaagctccagtgtctcatatatgcactggtaccaacaaaaacctggcagtagtcctcaggtgtggatctacgctacaagcaatctcgcttccggggttcccgtgaggtttagcggaagcgggtctggaactagttattccttgacaattagtcgggttgaagccgaggacaccgccacttactattgccaacagtggatattcaatccacccaccttcggttcaggtaccaagctcgaaatccgtggcggctccgagggcaagagcagcggcagcggcagcgagagcaagagcaccggcggcagccaagcatatctgcaacagagcggagctgagctggttcggcctggcgcctctgtaaaaatgagttgcaaggccagtggttatacattcacatcatataatatgcactgggtaaagcaaactccccgacaggggcttgaatggattggcgcaatctatcccggcaatggggatacatcctacaatcagaaattcaagggcaaggcaacactgaccgttgacaaatcctcatcaacagcctacatgcagctcagttccctcactagcgaagattctgctgtgtatttctgtgcaagggtgtattatggttctaattactggtatttcgatgtttggggaaccggaactaccgtaactgtttctagc

SEQ ID NO: 162 - protein, 737 aa, synthetic construct
  REGION 1..737: note = Description of Artificial Sequence: Synthetic polypeptide
QVQLVQSGAEVKKPGSSVKVSCKASGYTFTRSTMHWVRQAPGQGLEWMGYINPSSAYTNYAQKFQGRVTLTADKSTSTAYMELSSLRSEDTAVYYCASPQVHYDYGGFPYWGQGTLVTVSSGGSEGKSSGSGSESKSTGGSEIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSQIVLSQSPAILSASPGEKVTMTCRASLSVSSMHWYQQKPGSSPKPWIYATSNLASGVPARFSGSGSGTSYSLTISRVEAEDAATYYCQQWIFNPPTFGGGTKLEIKGGSEGKSSGSGSESKSTGGSQAYLQQSGAELVRPGASVKMSCKTSGYTFSSYNMHWVKQTPRQALEWIGAIYPGNGDTSYNQKFKGKATLTVDKSSSTAYMQLSSLTSEDSAVYFCTRSNYYGSSGWYFDVWGTGTTVTVSS

SEQ ID NO: 163 - DNA, 2211 bp, synthetic construct
  misc_feature 1..2211: note = Description of Artificial Sequence: Synthetic polynucleotide
  source 1..2211: mol_type = other DNA
caggtgcagctggtgcagagcggcgccgaggtgaagaagcctggcagcagcgtgaaggtgagctgtaaggccagcggctacactttcactaggagcactatgcactgggtgaggcaggcccctggccagggcctggagtggatgggctacatcaatcctagcagcgcctacactaattacgcccagaagttccagggcagggtgactctgactgccgataagagcactagcactgcctacatggagctgagcagcctgaggagcgaggatactgccgtgtactactgtgccagccctcaggtgcactacgattacggcggcttcccttactggggccagggcactctggtgactgtgagcagcggcggctccgagggcaagagcagcggcagcggcagcgagagcaagagcaccggcggcagcgagatcgtgctgactcagagccctgccactctgagcgccagccctggcgagagggtgactctgagctgtagcgccagcagcagcgtgagctacatgaattggtaccagcagaagcctggccaggcccctaggaggtggatctacgatagcagcaagctggccagcggcgtgcctgccaggttcagcggcagcggcagcggcagggattacactctgactatcagcagcctggagcctgaggatttcgccgtgtactactgtcagcagtggagcaggaatcctcctactttcggcggcggcactaaggtggagatcaaggagcccaaatctagcgacaaaactcacacatgtccaccgtgcccagcacctgaagcagcagggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagtctagatggcagcaggggaacgtcttctcatgctccgtgatgcatgaggctctgcacaaccactacacgcagaagagcctctccctgtctccgggtaaaggaggcggagggagtggcgggggaggctctcagattgtcctgagccaatccccagcaattctgagtgctagccctggagagaaggtaacaatgacttgtcgggcatcccttagcgtctcatccatgcattggtatcaacaaaagccaggttcatctccaaaaccctggatttacgctacatctaacctggcatctggggtgcctgccagatttagtggatctggttccggcacatcatattcccttacaatcagccgagtggaagccgaggatgctgcaacctattactgtcaacaatggatatttaaccctcccacctttgggggtgggactaaactcgaaatcaagggcggctccgagggcaagagcagcggcagcggcagcgagagcaagagcaccggcggcagccaagcctatcttcaacaatctggggctgagcttgtccggccaggagcctccgtcaaaatgagctgcaaaacctcaggttatacttttagtagctataacatgcattgggtaaaacaaaccccccgacaagcattggagtggataggggccatataccccggcaatggagacacaagttacaaccagaagtttaaaggcaaagctacactcacagttgacaaatcctcaagtactgcttatatgcaactctcctctctcacttccgaagacagtgccgtatatttttgcactcggtccaattactatggatctagtggctggtactttgacgtttggggcactgggacaactgttacagtgtccagc

SEQ ID NO: 164 - protein, 479 aa, synthetic construct
  REGION 1..479: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - DNA, 1437 bp, synthetic construct
  misc_feature 1..1437: note = Description of Artificial Sequence: Synthetic polynucleotide
  source 1..1437: mol_type = other DNA
gagatcgtgctgacccagtctcctgccacactgagtgcttctccaggcgagagagtgaccctgtcctgctccgcttcctcctccgtgtcctacatgaactggtatcagcagaagcccggccaggctcctcggagatggatctacgactcttccaagctggcctctggtgtgccagccagattttctggctctggctccggcagagactataccctgaccatctccagcctggaacctgaggacttcgccgtgtactactgccagcagtggtctaggaaccctcctacctttggcggaggcaccaaggtggaaatcaagggcggatctgagggaaagtccagcggctccggcagcgaaagcaagtccaccggcggaagccaggttcaactggttcagtctggcgccgaagtgaagaaacctggctcctccgtcaaggtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggtccactacgactacgccggctttccttattggggccagggcacactggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 166 - protein, 747 aa, synthetic construct
  REGION 1..747: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSGGGGSQIVLSQSPAILSASPGEKVTMTCRASLSVSSMHWYQQKPGSSPKPWIYATSNLASGVPARFSGSGSGTSYSLTISRVEAEDAATYYCQQWIFNPPTFGGGTKLEIKGGSEGKSSGSGSESKSTGGSQAYLQQSGAELVRPGASVKMSCKTSGYTFSSYNMHWVKQTPRQALEWIGAIYPGNGDTSYNQKFKGKATLTVDKSSSTAYMQLSSLTSEDSAVYFCTRSNYYGSSGWYFDVWGTGTTVTVSS

SEQ ID NO: 167 - DNA, 2241 bp, synthetic construct
  misc_feature 1..2241: note = Description of Artificial Sequence: Synthetic polynucleotide
  source 1..2241: mol_type = other DNA
gagatcgtgctgacccagtctcctgccacactgagtgcttctccaggcgagagagtgaccctgtcctgctccgcttcctcctccgtgtcctacatgaactggtatcagcagaagcccggccaggctcctcggagatggatctacgactcttccaagctggcctctggtgtgccagccagattttctggctctggctccggcagagactataccctgaccatctccagcctggaacctgaggacttcgccgtgtactactgccagcagtggtctaggaaccctcctacctttggcggaggcaccaaggtggaaatcaagggcggatctgagggaaagtccagcggctccggcagcgaaagcaagtccaccggcggaagccaggttcaactggttcagtctggcgccgaagtgaagaaacctggctcctccgtcaaggtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggtccactacgactacgccggctttccttattggggccagggcacactggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaaggaggcggaggatctggcggaggtggaagtggcggaggcggttctggtggtggtggatctcagatcgtgctgtctcagtctccagctatcctgtctgctagccctggcgagaaagtgaccatgacctgtagagccagcctgtccgtgtcctccatgcactggtatcagcagaagcctggcagctcccctaagccttggatctacgccacctccaatctggcctctggcgtgccagctagattctccggatctggctccggcacctcctacagcctgacaatctccagagtggaagccgaggatgccgccacctactactgtcagcagtggatcttcaaccctcctaccttcggcggaggcaccaagctggaaatcaagggagggagcgagggaaagtccagcggaagcggctctgagtccaaatccaccggagggagccaggcttacttgcagcagtctggtgccgaactcgttagacctggagcctccgtgaagatgtcctgcaagacctccggctacaccttctccagctacaacatgcactgggtcaagcagacccctcggcaggctctggaatggatcggcgctatctatcctggcaacggcgacacctcctacaaccagaagttcaagggcaaagctaccctgaccgtggacaagtcctcctccaccgcttacatgcagctgtccagcctgacctctgaggactccgccgtgtacttctgcacccggtctaactactacggctcctccggctggtacttcgatgtgtggggaaccggaaccaccgtgacagtctcttct

SEQ ID NO: 168 - protein, 743 aa, synthetic construct
  REGION 1..743: note = Description of Artificial Sequence: Synthetic polypeptide
DIQMTQSPSSLSASVGDRVTITCRARQSIGTAIHWYQQKPGKAPKLLIKYASESISGVPSRFSGSGSGTDFTLTISSLQPEDFATYYCQQSGSWPYTFGQGTKLEIKGGSEGKSSGSGSESKSTGGSEVQLVESGGGLVKPGGSLRLSCAASGFTFSRYNMNWVRQAPGKGLEWVSSISTSSNYIYYADSVKGRFTFSRDNAKNSLDLQMSGLRAEDTAIYYCTRGWGPFDYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIKGGSEGKSSGSGSESKSTGGSQVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSS

SEQ ID NO: 169 - DNA, 2229 bp, synthetic construct
  misc_feature 1..2229: note = Description of Artificial Sequence: Synthetic polynucleotide
  source 1..2229: mol_type = other DNA
gacatccagatgacccagtctccatcctctctgtccgcctctgtgggcgacagagtgaccattacctgccgggccagacagtctatcggcaccgctatccactggtatcagcagaagcctggcaaggcccctaagctgctgattaagtacgcctccgagtccatctccggcgtgccctccagattttctggctctggatctggcaccgactttaccctgacaatctccagcctgcagcctgaggacttcgccacctactactgtcagcagtccggctcttggccttacacctttggtcagggcaccaagctggaaatcaagggcggatctgagggaaagtccagcggctccggcagcgaaagcaagtccaccggcggaagcgaggtgcagctggttgaatctggcggaggactggttaagcctggcggctctctgagactgtcttgtgctgcttctggcttcaccttcagccggtacaacatgaactgggtccgacaggctcctggcaaaggcctggaatgggtgtcctccatctccacctccagcaactacatctactacgccgactccgtgaagggcagattcaccttctccagagacaacgccaagaactccctggacctgcagatgtctggcctgagagctgaggacaccgctatctactactgcaccagaggctggggacccttcgattattggggccagggaaccctggtcaccgtgtcatctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaaggaggcggaggatctggcggaggtggaagtggcggaggcggttctggtggtggtggatctgagatcgtgctgacccagtctccagccacactgtcactgtctccaggcgagagagctaccctgtcctgtagagcctctctgtccgtgtcctccatgcactggtatcagcagaagcctggacaggcccctcggctgctgatctacgctacctctaatctggccagcggtatccccgccagattttctggttctggctctggcaccgactttaccctgaccatctccagcctggaacctgaggacttcgccgtgtactactgccagcagtggatcttcaaccctcctacctttggcggaggcaccaaggtggaaatcaagggagggagcgagggaaagtccagcggaagcggctctgagtccaaatccaccggagggagccaggttcaactggttcagtctggcgccgaagtgaagaaacctggctcctccgtgaaggtgtcctgcaaggcttccggctacaccttctccagctacaacatgcactgggtccgacaggcccctggacaaggattggaatggatgggcgctatctaccctggcgctggcgatacctcttacgcccagaaattccagggcagagtgaccatcaccgccgacgagtctacctccaccgcctacatggaactgtccagcctgagatctgaggacaccgccgtgtactactgcgcccggtctaattactacggctccagcggctggtacttcgacgtgtggggaaagggcaccaccgtgacagtctcttct

SEQ ID NO: 170 - protein, 747 aa, synthetic construct
  REGION 1..747: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIKGGSEGKSSGSGSESKSTGGSQVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSS

SEQ ID NO: 171 - DNA, 2241 bp, synthetic construct
  misc_feature 1..2241: note = Description of Artificial Sequence: Synthetic polynucleotide
  source 1..2241: mol_type = other DNA
gagatcgtgctgacccagtctcctgccacactgagtgcttctccaggcgagagagtgaccctgtcctgctccgcttcctcctccgtgtcctacatgaactggtatcagcagaagcccggccaggctcctcggagatggatctacgactcttccaagctggcctctggtgtgccagccagattttctggctctggctccggcagagactataccctgaccatctccagcctggaacctgaggacttcgccgtgtactactgccagcagtggtctaggaaccctcctacctttggcggaggcaccaaggtggaaatcaagggcggatctgagggaaagtccagcggctccggcagcgaaagcaagtccaccggcggaagccaggttcaactggttcagtctggcgccgaagtgaagaaacctggctcctccgtcaaggtgtcctgcaaggcttccggctacacctttaccagatccaccatgcactgggtcaagcaggcccctggacaaggcttggagtggatcggctacatcaaccccagctccgcctacaccaactacaaccagaaattccagggcagagtgaccctgaccgccgacaagtctacctccaccgcctacatggaactgtccagcctgagatctgaggacaccgccgtgtactactgcgcctctcctcaggtccactacgactacgccggctttccttattggggccagggcacactggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaaggaggcggaggatctggcggaggtggaagtggcggaggcggttctggtggtggtggatctgagatcgtgctgacccagtctccagccacactgtcactgtctccaggcgagagagctaccctgtcctgtagagcctctctgtccgtgtcctccatgcactggtatcagcagaagcctggacaggcccctcggctgctgatctacgctacctctaatctggccagcggtatccccgccagattttctggttctggctctggcaccgactttaccctgaccatctccagcctggaacctgaggacttcgccgtgtactactgccagcagtggatcttcaaccctcctacctttggcggaggcaccaaggtggaaatcaagggagggagcgagggaaagtccagcggaagcggctctgagtccaaatccaccggagggagccaggttcaactggttcagtctggcgccgaagtgaagaaacctggctcctccgtgaaggtgtcctgcaaggcttccggctacaccttctccagctacaacatgcactgggtccgacaggcccctggacaaggattggaatggatgggcgctatctaccctggcgctggcgatacctcttacgcccagaaattccagggcagagtgaccatcaccgccgacgagtctacctccaccgcctacatggaactgtccagcctgagatctgaggacaccgccgtgtactactgcgcccggtctaattactacggctccagcggctggtacttcgacgtgtggggaaagggcaccaccgtgacagtctcttct

SEQ ID NO: 172 - protein, 451 aa, synthetic construct
  REGION 1..451: note = Description of Artificial Sequence: Synthetic polypeptide
QVQLQESGPGLVKPSETLSLTCSVSGASISSFYWSWIRQPADEGLEWIGRISPSGKTNYIPSLKSRIIMSLDASKNQFSLRLNSVTAADTAMYYCARGEYSGTYSYSFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 173 - DNA, 1353 bp, synthetic construct
  misc_feature 1..1353: note = Description of Artificial Sequence: Synthetic polynucleotide
  source 1..1353: mol_type = other DNA
caggttcagctgcaagagtctggtcctggcctggtcaagccttccgagacactgtctctgacctgctctgtgtccggcgcctccatctcttccttctactggtcctggatccggcagcctgctgacgaaggactggaatggatcggccggatcagcccttctggcaagaccaactacatccccagcctgaagtcccggatcatcatgtccctggacgcctccaagaaccagttctccctgcggctgaactctgtgaccgctgccgataccgccatgtactactgtgccagaggcgagtactccggcacctactcctacagctttgacgtgtggggacaaggcaccatggtcacagtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 174 - protein, 220 aa, synthetic construct
  REGION 1..220: note = Description of Artificial Sequence: Synthetic polypeptide
DIVMTQSPLSLSVTPGEPASISCRSSESLLDSEDGNTYLDWFLQKPGQSPQLLIYTLSYRASGVPDRFSGSGSDTDFTLHISSLEAEDVGLYYCMQRMEFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 175 - DNA, 660 bp, synthetic construct
  misc_feature 1..660: note = Description of Artificial Sequence: Synthetic polynucleotide
  source 1..660: mol_type = other DNA
gacatcgtgatgacccagtctccactgagcctgtctgtgacacctggcgagcctgcctccatctcctgtagatcttctgagtccctgctggacagcgaggacggcaatacctacctggactggttcctgcagaagcccggacagtctcctcagctgctgatctacaccctgtcctacagagcctctggcgtgcccgatagattctccggctctggctctgacaccgactttaccctgcacatctccagcctggaagccgaggatgtgggcctgtactactgtatgcagcggatggaatttcccctgaccttcggccagggcaccaaggtggaaatcaagcgcaccgtggccgcccctagcgtgtttatcttccctccctcggatgagcagcttaagtcaggcaccgcatccgtggtctgcctgctcaacaacttctacccgagggaagccaaagtgcagtggaaagtggacaacgcgctccagtcgggaaactcccaggagtccgtgaccgaacaggactccaaggacagcacttattccctgtcctccactctgacgctgtcaaaggccgactacgagaagcacaaggtctacgcctgcgaagtgacccatcaggggctttcctcgcccgtgactaagagcttcaatcggggcgaatgc

SEQ ID NO: 176 - protein, 449 aa, synthetic construct
  REGION 1..449: note = Description of Artificial Sequence: Synthetic polypeptide
QVQLQQSGPGLVKPSQTLSLTCAISGDSVSSNSATWNWIRQSPSRGLEWLGRTYYRSKWYNDYTVSVKSRITINPDTSKNQFSLQLNSVTPEDTAVYYCTRVDIAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 177 - DNA, 1347 bp, synthetic construct
  misc_feature 1..1347: note = Description of Artificial Sequence: Synthetic polynucleotide
  source 1..1347: mol_type = other DNA
caggttcagctgcagcagtctggccctggactggtcaagccctctcagaccctgtctctgacctgtgccatctccggcgactccgtgtcctctaattctgccacctggaactggatccggcagtcccctagtagaggcctggaatggctgggcagaacctactaccggtccaagtggtacaacgactacaccgtgtccgtgaagtcccggatcaccatcaatcccgacacctccaagaaccagttctccctgcagctcaacagcgtgacccctgaggataccgccgtgtactactgcaccagagtggatatcgccttcgactactggggccagggcacactggttaccgtttcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 178 - protein, 216 aa, synthetic construct
  REGION 1..216: note = Description of Artificial Sequence: Synthetic polypeptide
QTVVTQPPSVSEAPRQRVTISCSGSSSNIGNHGVNWYQQLPGKAPKLLIYNDDLLPSGVSDRFSGSTSGTSGSLAISGLQSEDEADYYCAAWDDSLNGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 179 - DNA, 648 bp, synthetic construct
  misc_feature 1..648: note = Description of Artificial Sequence: Synthetic polynucleotide
  source 1..648: mol_type = other DNA
cagacagtggtcacccagcctccatctgtgtctgaggcccctagacagagagtgaccatctcctgctccggctcctcctccaacatcggcaatcatggcgtgaactggtatcagcagctgcccggcaaggctcccaaactgctgatctacaacgacgacctgctgccttctggcgtgtccgacagattctccggctctacctctggcacctctggatccctggctatctctggcctgcagtctgaggacgaggccgactactattgtgccgcctgggacgattctctgaacggcgttgtgtttggcggaggcaccaagctgacagtgttgggacagcctaaggcagccccctccgtgaccctgttcccgccatcatccgaagaactgcaggccaacaaggccacgctcgtgtgcctgatttccgacttctacccgggggccgtgactgtggcctggaaggcagactcaagccctgtgaaggctggcgtcgagactaccaccccgtcgaagcaatccaacaacaaatacgcggcgtccagctacctgagcctgacccctgagcagtggaaatcccaccggtcctactcgtgccaagtcacccacgagggatccactgtggaaaagaccgtggcgccgactgagtgttcc

SEQ ID NO: 180 - protein, 451 aa, synthetic construct
  REGION 1..451: note = Description of Artificial Sequence: Synthetic polypeptide
QVQLQESGPGLVKPSQTLSLTCTVSGVSISNYYWSWIRQPPGKGLEWIGRISPSGRTNYNPSLKSRVTMSLDASKNQFSLKLSSVTAADTAVYYCARGEYSGTYSYSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 181 - DNA, 1353 bp, synthetic construct
  misc_feature 1..1353: note = Description of Artificial Sequence: Synthetic polynucleotide
  source 1..1353: mol_type = other DNA
caggttcagctgcaagagtctggccctggcctggtcaagccctctcagaccctgtctctgacctgtaccgtgtccggcgtgtccatctccaactactactggtcctggatccggcagcctcctggcaaaggactggaatggatcggccgcatctctccttctggtcgcaccaactacaaccccagcctgaaaagcagagtgaccatgtctctggacgcctccaagaaccagttctccctgaagctgtcctccgtgaccgctgctgataccgccgtgtactactgtgccagaggcgagtactccggcacctactcctacagcttcgacatctggggccagggcaccatggtcacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 182 - protein, 220 aa, synthetic construct
  REGION 1..220: note = Description of Artificial Sequence: Synthetic polypeptide
DIQMTQSPSSLSASVGDRVTITCRSSQSLFDSDDGNTYLDWFQQKPGQSPKLLIQTLSYRASGVPSRFSGSGSGTDFTLTISSLQPEDFATYYCMQRMEFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 183 - DNA, 660 bp, synthetic construct
  misc_feature 1..660: note = Description of Artificial Sequence: Synthetic polynucleotide
  source 1..660: mol_type = other DNA
gacatccagatgacccagtctccatcctctctgtccgcctctgtgggcgacagagtgaccatcacctgtcggtcctctcagtccctgttcgactctgacgacggcaacacctacctggactggttccagcagaagcccggccagtctcctaagctgctgatccagacactgtcctacagagcctctggcgtgccctccagattttccggctctggctctggcaccgactttaccctgacaatctccagcctgcagcctgaggacttcgccacctactactgtatgcagcggatggaatttcccctgaccttcggcggaggcaccaaggtggaaatcaagcgcaccgtggccgcccctagcgtgtttatcttccctccctcggatgagcagcttaagtcaggcaccgcatccgtggtctgcctgctcaacaacttctacccgagggaagccaaagtgcagtggaaagtggacaacgcgctccagtcgggaaactcccaggagtccgtgaccgaacaggactccaaggacagcacttattccctgtcctccactctgacgctgtcaaaggccgactacgagaagcacaaggtctacgcctgcgaagtgacccatcaggggctttcctcgcccgtgactaagagcttcaatcggggcgaatgc

SEQ ID NO: 184 - protein, 450 aa, synthetic construct
  REGION 1..450: note = Description of Artificial Sequence: Synthetic polypeptide
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 185 - DNA, 1350 bp, synthetic construct
  misc_feature 1..1350: note = Description of Artificial Sequence: Synthetic polynucleotide
  source 1..1350: mol_type = other DNA
cagatcactctgaaagagtccggcccaacactggtcaagcctacccagacactcacactgacctgtaccttcagcggattttctctgtccacctctggcatgggcgtgtcttggatcagacagcctcctggcaaggccctggaatggctggctcacatctactgggacgatgacaagcggtacaaccccagcctgaagtcccggctgaccatcaccaaggacacctccaagaaccaagtggtgctgaccatgaccaacatggaccctgtggataccgctacctactactgcgccagactgtacggcttcacctatggcttcgcctactggggccagggcaccctggtgaccgtgtcctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtgagcaagctcaccgtggacaagagcagatggcagcaggggaacgtcttctcatgctccgtgatgcatgaggctctgcacaaccggttcacgcagaagtctctctccctgtctccgggaaaa

SEQ ID NO: 186 - protein, 218 aa, synthetic construct
  REGION 1..218: note = Description of Artificial Sequence: Synthetic polypeptide
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 187 - DNA, 654 bp, synthetic construct
  misc_feature 1..654: note = Description of Artificial Sequence: Synthetic polynucleotide
  source 1..654: mol_type = other DNA
gatattgtgatgacccagtcccccgattctctcgctgtctctctgggcgaacgggctacaatcaactgtagggcttcacagtctgtcgactacaacggcatctcttacatgcattggtaccagcagaaacctggacagccaccaaaactcctcatctacgccgcttccaatcctgaatctggcgtgcccgaccgattttccggatccggctctggcaccgactttacactcactattagtagcctccaggccgaggatgtggccgtgtactactgtcagcagatcatcgaggatccttggacatttggacagggaaccaaagtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 188 - DNA, 660 bp, synthetic construct
  misc_feature 1..660: note = Description of Artificial Sequence: Synthetic polynucleotide
  source 1..660: mol_type = other DNA
gacatccagatgacccagagccctagcagcctgagcgccagcgtgggcgacagagtgaccattacctgcagaagcagccagagcctgttcgacagcgacgacggcaatacctacctggactggttccagcagaagcctggccagagccctaagctgctgatccagaccctgagctacagagccagcggcgtgcctagcagattctccggcagcggctccggcaccgacttcaccctgaccatcagcagcctgcagcctgaggacttcgccacctactactgcatgcagagaatggagttccctctgaccttcggcggcggcaccaaggtggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 189 - protein, 479 aa, synthetic construct
  REGION 1..479: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVRQAPGQGLEWMGYINPSSAYTNYAQKFQGRVTLTADKSTSTAYMELSSLRSEDTAVYYCASPQVHYDYGGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - DNA, 1437 bp, synthetic construct
  misc_feature 1..1437: note = Description of Artificial Sequence: Synthetic polynucleotide
  source 1..1437: mol_type = other DNA
gagatcgtgctgacccagtctcctgccacactgagtgcttctccaggcgagagagtgaccctgtcctgctccgcttcctcctccgtgtcctacatgaactggtatcagcagaagcccggccaggctcctcggagatggatctacgactcttccaagctggcctctggtgtgccagccagattttctggctctggctccggcagagactataccctgaccatctccagcctggaacctgaggacttcgccgtgtactactgccagcagtggtctaggaaccctcctacctttggcggaggcaccaaggtggaaatcaagggcggatctgagggaaagtccagcggctccggcagcgaaagcaagtccaccggcggaagccaggttcaactggttcagtctggcgccgaagtgaagaaacctggctcctccgtgaaagtgtcctgcaaggcttccggctacacttttaccagatccaccatgcactgggtccgacaggctccaggacaaggcttggagtggatgggctacatcaaccccagctccgcctacaccaactacgcccagaaattccagggcagagtgaccctgaccgccgacaagtctacctccaccgcctacatggaactgtccagcctgagatctgaggacaccgccgtgtactactgcgcttctcctcaggtgcactacgactacggcggctttccttattggggccagggcacactggtcaccgtttcttctgagcccaaatctagcgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 191 - protein, 451 aa, synthetic construct
  REGION 1..451: note = Description of Artificial Sequence: Synthetic polypeptide
QVQLQESGPGLVKPSQTLSLTCTVSGVSISNYYWSWIRQPPGKGLEWIGRISPSGRTNYNPSLKSRVTMSLDASKNQFSLKLSSVTAADTAVYYCARGEYSGTYSYSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 192 - DNA, 1353 bp, synthetic construct
  misc_feature 1..1353: note = Description of Artificial Sequence: Synthetic polynucleotide
  source 1..1353: mol_type = other DNA
caggttcagctgcaagagtctggccctggcctggtcaagccctctcagaccctgtctctgacctgtaccgtgtccggcgtgtccatctccaactactactggtcctggatccggcagcctcctggcaaaggactggaatggatcggccgcatctctccttctggtcgcaccaactacaaccccagcctgaaaagcagagtgaccatgtctctggacgcctccaagaaccagttctccctgaagctgtcctccgtgaccgctgctgataccgccgtgtactactgtgccagaggcgagtactccggcacctactcctacagcttcgacatctggggccagggcaccatggtcacagtctcttctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgccgggggaccgtcagtcttcctcttccccccaaaacccaaggacaccctcatgatctcccggacccctgaggtcacatgcgtggtggtgagcgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtgtcgaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcagatggcagcaggggaacgtcttctcatgctccgtgatgcatgaggctctgcacaaccactacacgcagaagtctctctccctgtctccgggaaaa

SEQ ID NO: 193 - protein, 12 aa, synthetic construct
  REGION 1..12: note = Description of Artificial Sequence: Synthetic peptide
GDSVFNNNAAWS

SEQ ID NO: 194 - protein, 11 aa, synthetic construct
  REGION 1..11: note = Description of Artificial Sequence: Synthetic peptide
RTYYRSKWLYD

SEQ ID NO: 195 - protein, 8 aa, synthetic construct
  REGION 1..8: note = Description of Artificial Sequence: Synthetic peptide
GYSSSFDY

SEQ ID NO: 196 - protein, 120 aa, synthetic construct
  REGION 1..120: note = Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 197 - protein, 450 aa, synthetic construct
  REGION 1..450: note = Description of Artificial Sequence: Synthetic polypeptide
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 198 - protein, 14 aa, synthetic construct
  REGION 1..14: note = Description of Artificial Sequence: Synthetic peptide
TGTSSNIGTYKFVS

SEQ ID NO: 199 - protein, 7 aa, synthetic construct
  REGION 1..7: note = Description of Artificial Sequence: Synthetic peptide
EVSKRPS

SEQ ID NO: 200 - protein, 10 aa, synthetic construct
  REGION 1..10: note = Description of Artificial Sequence: Synthetic peptide
VSYAGSGTLL

SEQ ID NO: 201 - protein, 110 aa, synthetic construct
  REGION 1..110: note = Description of Artificial Sequence: Synthetic polypeptide
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 202 - protein, 216 aa, synthetic construct
  REGION 1..216: note = Description of Artificial Sequence: Synthetic polypeptide
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 203 - protein, 5 aa, synthetic construct
  REGION 1..5: note = Description of Artificial Sequence: Synthetic peptide
TYAMN

SEQ ID NO: 204 - protein, 19 aa, synthetic construct
  REGION 1..19: note = Description of Artificial Sequence: Synthetic peptide
RIRSKYNNYATYYAASVKG

SEQ ID NO: 205 - protein, 14 aa, synthetic construct
  REGION 1..14: note = Description of Artificial Sequence: Synthetic peptide
HGNFGNSYVSWFAY

SEQ ID NO: 206 - protein, 125 aa, synthetic construct
  REGION 1..125: note = Description of Artificial Sequence: Synthetic polypeptide
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 207 - protein, 452 aa, synthetic construct
  REGION 1..452: note = Description of Artificial Sequence: Synthetic polypeptide
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFLLYSKLTVDKSRWQEGNVFSCSVMHEALHNHYTQKSLSLSLGK

SEQ ID NO: 208 - protein, 14 aa, synthetic construct
  REGION 1..14: note = Description of Artificial Sequence: Synthetic peptide
RSSTGAVTTSNYAN

SEQ ID NO: 209 - protein, 7 aa, synthetic construct
  REGION 1..7: note = Description of Artificial Sequence: Synthetic peptide
GTNKRAP

SEQ ID NO: 210 - protein, 9 aa, synthetic construct
  REGION 1..9: note = Description of Artificial Sequence: Synthetic peptide
ALWYSNLWV

SEQ ID NO: 211 - protein, 109 aa, synthetic construct
  REGION 1..109: note = Description of Artificial Sequence: Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 212 - protein, 215 aa, synthetic construct
  REGION 1..215: note = Description of Artificial Sequence: Synthetic polypeptide
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 213 - DNA, 339 bp, synthetic construct
  misc_feature 1..339: note = Description of Artificial Sequence: Synthetic polynucleotide
  source 1..339: mol_type = other DNA
gatattgtgatgactcagtctccactctccctgtccgtcacccctggagagccggcctccatctcctgcaggtctagtgagagcctcttggatagtgaagatggaaacacctatttggactggttcctgcagaagccagggcagtctcctcagctcctgatctatacgctttcctatcgggcctctggagtcccagacaggttcagtggcagtgggtcggacactgatttcacactgcacatcagcagtctggaggctgaggatgttggactttattactgcatgcaacgtatggagtttccgctcactttcggccaagggaccaaggtggaaatcaaa

SEQ ID NO: 214 - DNA, 339 bp, synthetic construct
  misc_feature 1..339: note = Description of Artificial Sequence: Synthetic polynucleotide
  source 1..339: mol_type = other DNA
gacatccagatgacccagagccctagcagcctgagcgccagcgtgggcgacagagtgaccattacctgcagaagcagccagagcctgttcgacagcgacgacggcaatacctacctggactggttccagcagaagcctggccagagccctaagctgctgatccagaccctgagctacagagccagcggcgtgcctagcagattctccggcagcggctccggcaccgacttcaccctgaccatcagcagcctgcagcctgaggacttcgccacctactactgcatgcagagaatggagttccctctgaccttcggcggcggcaccaaggtggagatcaag

SEQ ID NO: 215 - protein, 20 aa, synthetic construct
  REGION 1..20: note = Description of Artificial Sequence: Synthetic peptide
GGSEGKSSGSGSESKSTGGS

SEQ ID NO: 216 - protein, 8 aa, synthetic construct
  REGION 1..8: note = Description of Artificial Sequence: Synthetic peptide
GGGSGGGS

SEQ ID NO: 217 - protein, 12 aa, synthetic construct
  REGION 1..12: note = Description of Artificial Sequence: Synthetic peptide
GGGSGGGSGGGS

SEQ ID NO: 218 - protein, 16 aa, synthetic construct
  REGION 1..16: note = Description of Artificial Sequence: Synthetic peptide
GGGSGGGSGGGSGGGS

SEQ ID NO: 219 - protein, 20 aa, synthetic construct
  REGION 1..20: note = Description of Artificial Sequence: Synthetic peptide
GGGSGGGSGGGSGGGSGGGS

SEQ ID NO: 220 - protein, 15 aa, synthetic construct
  REGION 1..15: note = Description of Artificial Sequence: Synthetic peptide
GGGGSGGGGSGGGGS

SEQ ID NO: 221 - protein, 20 aa, synthetic construct
  REGION 1..20: note = Description of Artificial Sequence: Synthetic peptide
GGGGSGGGGSGGGGSGGGGS

SEQ ID NO: 222 - protein, 25 aa, synthetic construct
  REGION 1..25: note = Description of Artificial Sequence: Synthetic peptide
GGGGSGGGGSGGGGSGGGGSGGGGS

SEQ ID NO: 223 - protein, 18 aa, synthetic construct
  REGION 1..18: note = Description of Artificial Sequence: Synthetic peptide
GSTSGSGKPGSGEGSTKG

SEQ ID NO: 224 - protein, 14 aa, synthetic construct
  REGION 1..14: note = Description of Artificial Sequence: Synthetic peptide
IRPRAIGGSKPRVA

SEQ ID NO: 225 - protein, 15 aa, synthetic construct
  REGION 1..15: note = Description of Artificial Sequence: Synthetic peptide
GKGGSGKGGSGKGGS

SEQ ID NO: 226 - protein, 15 aa, synthetic construct
  REGION 1..15: note = Description of Artificial Sequence: Synthetic peptide
GGKGSGGKGSGGKGS

SEQ ID NO: 227 - protein, 15 aa, synthetic construct
  REGION 1..15: note = Description of Artificial Sequence: Synthetic peptide
GGGKSGGGKSGGGKS

SEQ ID NO: 228 - protein, 15 aa, synthetic construct
  REGION 1..15: note = Description of Artificial Sequence: Synthetic peptide
GKGKSGKGKSGKGKS

SEQ ID NO: 229 - protein, 15 aa, synthetic construct
  REGION 1..15: note = Description of Artificial Sequence: Synthetic peptide
GGGKSGGKGSGKGGS

SEQ ID NO: 230 - protein, 15 aa, synthetic construct
  REGION 1..15: note = Description of Artificial Sequence: Synthetic peptide
GKPGSGKPGSGKPGS

SEQ ID NO: 231 - protein, 20 aa, synthetic construct
  REGION 1..20: note = Description of Artificial Sequence: Synthetic peptide
GKPGSGKPGSGKPGSGKPGS

SEQ ID NO: 232 - protein, 20 aa, synthetic construct
  REGION 1..20: note = Description of Artificial Sequence: Synthetic peptide
GKGKSGKGKSGKGKSGKGKS

SEQ ID NO: 233 - protein, 14 aa, synthetic construct
  REGION 1..14: note = Description of Artificial Sequence: Synthetic peptide
STAGDTHLGGEDFD

SEQ ID NO: 234 - protein, 15 aa, synthetic construct
  REGION 1..15: note = Description of Artificial Sequence: Synthetic peptide
GEGGSGEGGSGEGGS

SEQ ID NO: 235 - protein, 15 aa, synthetic construct
  REGION 1..15: note = Description of Artificial Sequence: Synthetic peptide
GGEGSGGEGSGGEGS

SEQ ID NO: 236 - protein, 15 aa, synthetic construct
  REGION 1..15: note = Description of Artificial Sequence: Synthetic peptide
GEGESGEGESGEGES

SEQ ID NO: 237 - protein, 15 aa, synthetic construct
  REGION 1..15: note = Description of Artificial Sequence: Synthetic peptide
GGGESGGEGSGEGGS

SEQ ID NO: 238 - protein, 20 aa, synthetic construct
  REGION 1..20: note = Description of Artificial Sequence: Synthetic peptide
GEGESGEGESGEGESGEGES

SEQ ID NO: 239 - protein, 18 aa, synthetic construct
  REGION 1..18: note = Description of Artificial Sequence: Synthetic peptide
GSTSGSGKPGSGEGSTKG

SEQ ID NO: 240 - protein, 19 aa, synthetic construct
  REGION 1..19: note = Description of Artificial Sequence: Synthetic peptide
PRGASKSGSASQTGSAPGS

SEQ ID NO: 241 - protein, 19 aa, synthetic construct
  REGION 1..19: note = Description of Artificial Sequence: Synthetic peptide
GTAAAGAGAAGGAAAGAAG

SEQ ID NO: 242 - protein, 19 aa, synthetic construct
  REGION 1..19: note = Description of Artificial Sequence: Synthetic peptide
GTSGSSGSGSGGSGSGGGG

SEQ ID NO: 243 - protein, 20 aa, synthetic construct
  REGION 1..20: note = Description of Artificial Sequence: Synthetic peptide
GKPGSGKPGSGKPGSGKPGS

SEQ ID NO: 244 - protein, 4 aa, synthetic construct
  REGION 1..4: note = Description of Artificial Sequence: Synthetic peptide
GSGS

SEQ ID NO: 245 - protein, 10 aa, synthetic construct
  REGION 1..10: note = Description of Artificial Sequence: Synthetic peptide
APAPAPAPAP

SEQ ID NO: 246 - protein, 20 aa, synthetic construct
  REGION 1..20: note = Description of Artificial Sequence: Synthetic peptide
APAPAPAPAPAPAPAPAPAP

SEQ ID NO: 247 - protein, 32 aa, synthetic construct
  REGION 1..32: note = Description of Artificial Sequence: Synthetic polypeptide
AEAAAKEAAAKEAAAAKEAAAAKEAAAAKAAA

SEQ ID NO: 248 - protein, 10 aa, synthetic construct
  REGION 1..10: note = Description of Artificial Sequence: Synthetic peptide
GGGGSGGGGS

SEQ ID NO: 249 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 250 - protein, 330 aa, synthetic construct
  REGION 1..330: note = Description of Artificial Sequence: Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 251 - protein, 327 aa, synthetic construct
  REGION 1..327: note = Description of Artificial Sequence: Synthetic polypeptide
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 252 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 253 - protein, 13 aa, Homo sapiens
SEDRYRNPKGSAC

SEQ ID NO: 254 - protein, 5 aa, Homo sapiens
EMENP

SEQ ID NO: 255 - protein, 5 aa, Homo sapiens
GFSTL

SEQ ID NO: 256 - protein, 14 aa, Homo sapiens
QDGNEEMGGITQTP

SEQ ID NO: 257 - protein, 5 aa, Homo sapiens
GSEIL

SEQ ID NO: 258 - protein, 6 aa, Homo sapiens
PRGSKP

SEQ ID NO: 259 - protein, 1418 aa, synthetic construct
  REGION 1..1418: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIKGGSEGKSSGSGSESKSTGGSQVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSQVQLQESGPGLVKPSETLSLTCSVSGASISSFYWSWIRQPADEGLEWIGRISPSGKTNYIPSLKSRIIMSLDASKNQFSLRLNSVTAADTAMYYCARGEYSGTYSYSFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKDIVMTQSPLSLSVTPGEPASISCRSSESLLDSEDGNTYLDWFLQKPGQSPQLLIYTLSYRASGVPDRFSGSGSDTDFTLHISSLEAEDVGLYYCMQRMEFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 748 aa, synthetic construct
  REGION 1..748: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIKGGSEGKSSGSGSESKSTGGSQVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSH

SEQ ID NO: 261 - protein, 451 aa, synthetic construct; identical to SEQ ID NO: 172
  REGION 1..451: note = Description of Artificial Sequence: Synthetic polypeptide

SEQ ID NO: 262 - protein, 220 aa, synthetic construct; identical to SEQ ID NO: 174
  REGION 1..220: note = Description of Artificial Sequence: Synthetic polypeptide

SEQ ID NO: 263 - protein, 1419 aa, synthetic construct
  REGION 1..1419: note = Description of Artificial Sequence: Synthetic polypeptide
EIVLTQSPATLSASPGERVTLSCSASSSVSYMNWYQQKPGQAPRRWIYDSSKLASGVPARFSGSGSGRDYTLTISSLEPEDFAVYYCQQWSRNPPTFGGGTKVEIKGGSEGKSSGSGSESKSTGGSQVQLVQSGAEVKKPGSSVKVSCKASGYTFTRSTMHWVKQAPGQGLEWIGYINPSSAYTNYNQKFQGRVTLTADKSTSTAYMELSSLRSEDTAVYYCASPQVHYDYAGFPYWGQGTLVTVSSEPKSSDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSEIVLTQSPATLSLSPGERATLSCRASLSVSSMHWYQQKPGQAPRLLIYATSNLASGIPARFSGSGSGTDFTLTISSLEPEDFAVYYCQQWIFNPPTFGGGTKVEIKGGSEGKSSGSGSESKSTGGSQVQLVQSGAEVKKPGSSVKVSCKASGYTFSSYNMHWVRQAPGQGLEWMGAIYPGAGDTSYAQKFQGRVTITADESTSTAYMELSSLRSEDTAVYYCARSNYYGSSGWYFDVWGKGTTVTVSSHQVQLQESGPGLVKPSETLSLTCSVSGASISSFYWSWIRQPADEGLEWIGRISPSGKTNYIPSLKSRIIMSLDASKNQFSLRLNSVTAADTAMYYCARGEYSGTYSYSFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGKDIVMTQSPLSLSVTPGEPASISCRSSESLLDSEDGNTYLDWFLQKPGQSPQLLIYTLSYRASGVPDRFSGSGSDTDFTLHISSLEAEDVGLYYCMQRMEFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 264 - protein, 9 aa, Homo sapiens
IPAGIYAPI

SEQ ID NO: 265 - protein, 44 aa, Homo sapiens
HFLKMESLNFIRAHTPYINIYNCEPANPSEKNSPSTQYCYSIQS

(19) United States
(12) Patent Application Publication
(10) Pub. No.: US 2025/0043019 A1
(43) Pub. Date: Feb. 6, 2025

(54) TRISPECIFIC ANTIBODY TARGETING CD79b, CD20, AND CD3

(71) Applicant: Janssen Biotech, Inc., Horsham, PA (US)

(72) Inventors: Rajkumar Ganesan, Blue Bell, PA (US); Anna Kuchnio, Edegem (BE); Cassandra L. Lowenstein, Ambler, PA (US); Ulrike Philippar, Antwerpen (BE); Sanjaya Singh, Blue Bell, PA (US); Nele Vloemans, Oostmalle (BE); Danlin Yang, Philadelphia, PA (US)

(21) Appl. No.: 18/811,332

(22) Filed: Aug. 21, 2024

Related U.S. Application Data
(63) Continuation of application No. 17/702,282, filed on Mar. 23, 2022, now Pat. No. 12,103,977.
(60) Provisional application No. 63/286,309, filed on Dec. 6, 2021.
(60) Provisional application No. 63/165,501, filed on Mar. 24, 2021.

(51) Int. Cl. C07K 16/28; A61K 39/00; A61P 35/00

(52) U.S. Cl. CPC C07K 16/2896; A61P 35/00; C07K 16/2878; C07K 16/2887; A61K 2039/505; C07K 2317/24; C07K 2317/31; C07K 2317/55; C07K 2317/565; C07K 2317/622; C07K 2317/73; C07K 2317/76; C07K 2317/92

(57) ABSTRACT

Provided herein are multispecific antibodies, including trispecific antibodies that bind to CD79b, CD20 and CD3, and bispecific antibodies that bind to CD79b and CD3, and multispecific antigen-binding fragments thereof. Also described are related polynucleotides capable of encoding the provided multispecific antibodies or multispecific antigen-binding fragments, cells expressing the provided multispecific antibodies or multispecific antigen-binding fragments, as well as associated vectors and detectably labeled multispecific antibodies or multispecific antigen-binding fragments. In addition, methods of producing and using the provided multispecific antibodies and multispecific antigen-binding fragments are described. Further provided herein are isolated antibodies that bind to CD79b and antigen-binding fragments thereof. Also described are related polynucleotides capable of encoding the provided CD79b-specific antibodies or antigen-binding fragments, cells expressing the provided CD79b-specific antibodies or antigen-binding fragments, as well as associated vectors and detectably labeled CD79b-specific antibodies or antigen-binding fragments. In addition, methods of producing and using the provided CD79b-specific antibodies and antigen-binding fragments are described.

### CROSS-REFERENCE TO RELATED APPLICATIONS

This application claims the benefit of U.S. Provisional Application Ser. No. 63/165,501, filed 24 Mar. 2021 and U.S. Provisional Application Ser. No. 63/286,309, filed 6 Dec. 2021. The entire content of the aforementioned applications is incorporated herein by reference in its entirety.

### SEQUENCE LISTING

The instant application contains a sequence listing which has been submitted electronically in ASCII format and is hereby incorporated by reference in its entirety. Said ASCII copy, created on Jan. 28, 2022, is named PRD4135USNP1_SL.txt and is 357,539 bytes in size.

### TECHNICAL FIELD

The disclosure provided herein relates to multispecific antibodies that bind cluster of differentiation 79B protein (CD79b), cluster of differentiation 20 (CD20), and cluster of differentiation 3 (CD3), bispecific antibodies that bind CD79b and CD3, monoclonal antibodies that bind CD79b, as well as methods of producing and using the described antibodies.

### BACKGROUND

Non-Hodgkin lymphoma (NHL) accounts for about 4% of all cancers. Despite improvements in available therapies, relapsed/refractory (R/R, sometimes referred to as r/r) NHLs are characterized by uniformly poor prognosis. Adoptive immunotherapy using T cells genetically engineered to express a chimeric antigen receptor (CAR) has shown promising results for the treatment of CD19-positive B cell malignancies. However, even with an initial overall response rate of approximately 60-80%, only 40% of patients achieve long-term, complete remission [1, 2]. There is emerging clinical data indicating disease relapse due to CD19 antigen loss in both acute lymphoblastic leukemia (ALL) and diffuse large B-cell lymphoma (DLBCL) patients [2, 4]. Accordingly, there is a need for targeting novel surface antigens.

T-cell redirection is a powerful and novel treatment that may address unmet medical needs of patients with B-cell malignancies whose disease no longer responds to standard chemo- or immunotherapies. CD20xCD3 and CD19xCD3 bispecific antibodies, show promising clinical response rates (10-12), and provide proof of concept (PoC) that this therapeutic approach can be highly effective in the clinical management of B-cell malignancies.

B cells, or B lymphocytes, are central components of adaptive immunity, responding to several different pathogens by producing antibodies, performing the role of antigen-presenting cells, secreting cytokines, and developing into memory B cells after activation [5]. B cells circulate in the blood and lymphatic systems. In the lymphoid organs, a B cell encounters its cognate antigen, and together with an additional signal from a T helper cell, the B cell can differentiate into effector plasma cells. These cells secrete specific antibodies that will circulate in the blood to target and eliminate antigens or pathogens [6].

To detect the antigen or pathogen, B cells have B cell receptors (BCRs) on the cell surface. The B-cell receptor is a multicomponent receptor composed of a transmembrane immunoglobulin molecule (mIg) and a disulfide linked heterodimer of CD79a (Igα) and CD79b (Igβ). CD79b is highly expressed in a wide range of B-cell lymphomas. Its expression plays a role in cancer cell viability of most DLBCL tumor models. Therefore, the development of resistance to CD79b targeted agents through antigen loss may be less likely, making it an attractive target for the development of novel immunotherapeutic approaches. In the clinic, Polatuzumab (POLIVY™), an antibody-drug conjugate (ADC) molecule targeting CD79b, has recently been approved for the treatment of r/r DLBCL [7]. Polatuzumab treatments results in an increase in complete response (CR) and duration of response (DOR) rates when combined with standard of care treatments (bendamustine and Rituximab), validating CD79b as a valuable clinical target [8].

The CD20 molecule (also called human B-lymphocyte-restricted differentiation antigen or Bp35) is a hydrophobic transmembrane protein that is over-expressed on most hematological malignancies in B cell linages. CD20 is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin's lymphomas (NHL) (Anderson et al. (1984) Blood 63(6):1424-1433).

Targeting more than one lymphoma tumor antigen and engaging T-cells could lead to efficient killing of malignant plasma cells and minimal residual disease (MRD) negativity. Dual CD79b and CD20 targeting prevents tumor antigen escape, targets clonal populations (e.g., captures tumor cells that do not express enough CD79b or CD20 alone), improves tumor efficacy through avidity effects. The use of a low-affinity CD3-engaging arm may mitigate the potential risk of cytokine release syndrome (CRS). Accordingly, there is a need for therapeutic antibodies that target both CD79b and CD20 for the treatment of B-cell malignancies such as B-cell lymphomas and non-Hodgkin's lymphomas.

### SUMMARY

In one aspect, provided herein are multispecific antibodies that bind, or specifically bind, to CD79b, CD20 and/or CD3 and multispecific antigen-binding fragments thereof. In some embodiments, provided herein are trispecific antibodies that bind, or specifically bind, to CD79b, CD20 and CD3 (“CD79b x CD20 x CD3”) and trispecific antigen-binding fragments thereof. In some embodiments, provided herein are bispecific antibodies that bind to CD79b and CD3 (“CD79b x CD3”), and bispecific antigen-binding fragments thereof. Also described are related polynucleotides capable of encoding the provided multispecific antibodies or multispecific antigen-binding fragments, cells expressing the provided multispecific antibodies or multispecific antigen-binding fragments, as well as associated vectors and detectably labeled multispecific antibodies or multispecific antigen-binding fragments. In addition, methods of using the provided multispecific antibodies are described. For example, the multispecific antibodies and multispecific antigen-binding fragments may be used to treat cancer (e.g., CD79b and/or CD20-expressing cancer); the multispecific antibodies may be used to diagnose or monitor CD79b and/or CD20-expressing cancer progression, regression, or stability; to determine whether or not a patient should be treated for cancer; or to determine whether or not a subject is afflicted with CD79b and/or CD20-expressing cancer and thus may be amenable to treatment with a CD79b and/or CD20-specific anti-cancer therapeutic, such as the CD79b x CD20 x CD3 trispecific antibodies, or the CD79b x CD3 bispecific antibodies described herein.

The redirection of T-lymphocytes to tumor cells expressing CD79b and/or CD20 via the TCR/CD3 complex represents an attractive alternative approach. The TCR/CD3 complex of T-lymphocytes consists of either a TCR alpha (α)/beta (β) or TCR gamma (γ)/delta (δ) heterodimer coexpressed at the cell surface with the invariant subunits of CD3 labeled gamma (γ), delta (δ), epsilon (ε), zeta (ξ), and eta (η). In some embodiments, the multispecific antibodies or multispecific antigen-binding fragments described herein specifically bind to CD3ε.

### CD79b x CD20 x CD3-Trispecific Antibodies

In some embodiments, provided herein are isolated CD79b x CD20 x CD3 trispecific antibody or antigen-binding fragment. In some embodiments, an isolated CD79b x CD20x CD3 trispecific antibody, or a trispecific binding fragment thereof, comprises: a) a first antigen-binding arm comprising a first heavy chain variable domain (VH1) and a first light chain variable domain (VL1); (b) a second antigen-binding arm comprising a second heavy chain variable domain (VH2) and a second light chain variable domain (VL2); and (c) a third antigen-binding arm comprising a third heavy chain variable domain (VH3) and a third light chain variable domain (VL3). In some embodiments, the first antigen binding arm binds to an epitope on CD79b; the second antigen binding arm binds to an epitope on CD3, and the third antigen binding arm binds to an epitope on CD20.

According to all aspects of the invention the CD79b x CD20 x CD3 trispecific antibody or antigen-binding fragment may bind to a conformational epitope of CD79 made up of residues 30-42 (SEDRYRNPKGSAC; SEQ ID NO: 253), 50-52 (PRF), 81-86 (EMENP; SEQ ID NO: 254), and 144-148 (GFSTL; SEQ ID NO: 255). The residue numbers are those of human CD79B (P40259).

According to all aspects of the invention the CD79b x CD20 x CD3 trispecific antibody or antigen-binding fragment may bind to a conformational epitope of CD3 encompassing residues 54-58 (GSEIL; SEQ ID NO: 257), 74-75 (NI), and 100-105 (PRGSKP; SEQ ID NO: 258). The residue numbers are those of human CD3E (P07766).

In some embodiments, an isolated CD79b x CD20 x CD3 trispecific antibody, or a trispecific binding fragment thereof, comprises: a) a first heavy chain portion (HC1); b) a light chain portion; c) a second heavy chain portion (HC2), wherein the HC1 and the LC form a first antigen-binding site that specifically binds a first antigen, the HC2 comprises a second antigen-binding site that specifically binds a second antigen, the HC1 or the HC2 further comprises a third antigen-binding site that specifically binds a third antigen, and the HC1 and HC2 each comprise a Fragment crystallizable (Fc) domain comprising a CH2-CH3 domain. In some embodiments, the first antigen-binding arm of the trispecific antibody, or a trispecific binding fragment thereof comprises a first heavy chain portion (HC1) comprising the VH1, and a light chain portion (LC) comprising the VL1. The VH1 and the VL1 form a first antigen-binding domain that binds a first antigen. The second antigen-binding arm of the trispecific antibody or trispecific binding fragment thereof comprises a second heavy chain portion (HC2) comprising the VH2 domain. The VH2 domain of the HC2 forms a second antigen-binding domain that binds a second antigen. The HC1 or the HC2 is further coupled to the third antigen-binding arm comprising the VH3 that forms a third antigen-binding domain that binds a third antigen. The HC1 and HC2 each optionally comprise a Fragment crystallizable (Fc) domain, where the Fc domain comprises a constant heavy chain region 2 (CH2) and CH3. In some embodiments, the first antigen is cluster of differentiation 79b (CD79b), and the second antigen is cluster of differentiation 3 (CD3), and the third antigen is cluster of differentiation 20 (CD20). In some embodiments, the first antigen is cluster of differentiation 79b (CD79b), and the second antigen is cluster of differentiation 20 (CD20), and the third antigen is cluster of differentiation 3 (CD3).

Some aspects of the CD79b x CD20 x CD3 trispecific antibody, or a trispecific binding fragment thereof, are further described in the Detail Description and Examples sections below.

In some embodiments, the CD79b-binding arm (or “CD79b-specific arm”) of the CD79b x CD20 x CD3 trispecific antibody is derived from a CD79b antibody described herein (for example, from an antibody having the CDR sequences listed in Table 1a). In some embodiments, the CD79b-binding arm of the CD79b x CD20 x CD3 trispecific antibody comprises any one variable heavy (VH) domain and any one variable light (VL) domain selected from Table 1b. In some embodiments, the CD79b-binding arm of the CD79b x CD20 x CD3 trispecific antibody is derived from the CD79b antibody CD9B374 as described herein.

In some embodiments, the CD20-binding arm (or “CD20-specific arm”) of the CD79b x CD20 x CD3 trispecific antibody is derived from a CD20 antibody described herein (for example, from an antibody having the CDR sequences listed in Table 2a). In some embodiments, the CD20-binding arm of the CD79b x CD20 x CD3 trispecific antibody comprises any one VH domain and any one VL domain selected from Table 2b. In some embodiments, the CD20-binding arm of the CD79b x CD20 x CD3 trispecific antibody is derived from a CD20 antibody C20B648.

In some embodiments, the CD3-binding arm (or “CD3-specific arm”) of the CD79b x CD20 x CD3 trispecific antibody is derived from a CD3 antibody described herein (for example, from an antibody having the CDR sequences listed in Table 3). In some embodiments, the CD3-binding arm of the CD79b x CD20 x CD3 trispecific antibody comprises any one VH domain and any one VL domain selected from Table 3. In some embodiments, the CD3-binding arm of the CD79b x CD20 x CD3 trispecific antibody is derived from the monoclonal antibody CD3W245. In some embodiments, the CD3-binding arm of the CD79b x CD20 x CD3 trispecific antibody is derived from the monoclonal antibody CD3B2030.

In some embodiments, the CD79b-, CD20- and/or CD3-specific arms of the CD79b x CD20x CD3-multispecific antibodies or antigen-binding fragments are IgG, or derivatives thereof. The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcγRs) on the surface of immune effector cells such as natural killers and macrophages, leading to the phagocytosis or lysis of the targeted cells. In CDC, the antibodies kill the targeted cells by triggering the complement cascade at the cell surface.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcγRs or the complement factors. The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Mutations have been introduced in IgG1, IgG2 and IgG4 to reduce or silence Fc functionalities.

In one embodiment, the antibody comprises an Fc region with one or more of the following properties: (a) reduced effector function when compared to the parent Fc; (b) reduced affinity to FcγRI, FcγRIIa, FcγRIIb, FcγRIIIb and/or FcγRIIIa, (c) reduced affinity to FcγRI (d) reduced affinity to FcγRIIa (e) reduced affinity to FcγRIIb, (f) reduced affinity to FcγRIIIb or (g) reduced affinity to FcγRIIIa.

In some embodiments, the CD3-specific antibody or antigen-binding fragment from which the CD3-specific arm of the trispecific antibody is derived is IgG, or a derivative thereof. In some embodiments, the CD3-specific antibody or antigen-binding fragment from which the CD3-specific arm of the trispecific antibody is derived is IgG1, or a derivative thereof. In some embodiments, for example, the Fc region of the CD3-specific IgG1 antibody from which the CD3-binding arm is derived comprises L234A, L235A, and D265S substitutions in its Fc region. In some embodiments, the CD3-specific antibody or antigen-binding fragment from which the CD3-specific arm of the trispecific antibody is derived is IgG4, or a derivative thereof. In some embodiments, for example, the Fc region of the CD3-specific IgG4 antibody from which the CD3-binding arm is derived comprises S228P, L234A, L235A, F405L, and R409K substitutions in its Fc region. In some embodiments, the CD3-specific antibody or antigen-binding fragment from which the CD3-specific arm of the trispecific antibody is derived binds CD3ε on primary human T cells and/or primary cynomolgus T cells. In some embodiments, the CD3-specific antibody or antigen-binding fragment from which the CD3-specific arm of the trispecific antibody is derived activates primary human CD4+ T cells and/or primary cynomolgus CD4+ T cells.

In addition to the described CD79b x CD20 x CD3-multispecific antibodies, also provided are polynucleotide sequences capable of encoding the described CD79b x CD20x CD3-multispecific antibodies. In some embodiments, an isolated synthetic polynucleotide encoding the one or more CDRs of the heavy chain variable domain and/or one or more CDRs of the light chain variable domain of each antigen-binding arm of the CD79b x CD20 x CD3 trispecific antibody or trispecific binding fragment is provided. In some embodiments, an isolated synthetic polynucleotide encoding one or more heavy chain variable domains (such as the HC1 and/or the HC2) and/or one or more light chain variable domains of the CD79b x CD20 x CD3 trispecific antibody or trispecific binding fragment is provided. In some embodiments, an isolated synthetic polynucleotide encoding one or more polypeptide chains of the first, second, and/or third antigen-binding arms of the CD79b x CD20 x CD3 trispecific antibody or trispecific binding fragment is provided. Vectors comprising the described polynucleotides are also provided, as are cells expressing the CD79b x CD20 x CD3-multispecific antibodies provided herein. In another embodiment, an isolated cell expressing the trispecific antibody or trispecific binding fragment is provided. Also described are cells capable of expressing the disclosed vectors. These cells may be mammalian cells (such as 293 cells, 293F cells, CHO cells), insect cells (such as Sf7 cells), yeast cells, plant cells, or bacteria cells (such as E. coli). The described antibodies may also be produced by hybridoma cells. In some embodiments, methods for generating the CD79b x CD20 x CD3 trispecific antibody or trispecific binding fragment by culturing cells is provided.

Further provided herein are pharmaceutical compositions comprising the CD79b x CD20 x CD3 trispecific antibodies or antigen-binding fragments and a pharmaceutically acceptable carrier.

### Methods of Using CD79B x CD20 x CD3− Trispecific Antibodies

Methods of using the described CD79b x CD20 x CD3-trispecific antibodies and trispecific antigen-binding fragments thereof are also disclosed. For example, the CD79b x CD20 x CD3-multispecific antibodies and trispecific antigen-binding fragments thereof may be useful in the treatment of a CD79b and/or CD20-expressing cancer in a subject in need thereof. In some embodiments, the CD79b and/or CD20-expressing cancer is a lymphoma, such as diffuse large B-cell lymphoma (DLBCL), mantle cell lymphoma (MCL), marginal zone lymphoma (MZL), follicular lymphoma (FL), chronic lymphocytic leukemia (CLL), or Waldenström macroglobulinemia (WM). In some embodiments, the CD79b and/or CD20-expressing cancer is a relapsed or refractory form of lymphoma, such as a relapsed or refractory form of diffuse large B-cell lymphoma (DLBCL), mantle cell lymphoma (MCL), marginal zone lymphoma (MZL), follicular lymphoma (FL), chronic lymphocytic leukemia (CLL), or Waldenström macroglobulinemia (WM).

The described methods of treating CD79b and/or CD20-expressing cancer in a subject in need thereof include administering to the subject a therapeutically effective amount of a described CD79b x CD20 x CD3-trispecific antibody or trispecific antigen-binding fragment thereof. In some embodiments, the subject is a mammal, preferably a human. In preferred embodiments are provided methods for treating a subject having cancer by administering a therapeutically effective amount of the CD79b x CD20 x CD3 trispecific antibody or trispecific antigen-binding fragment to a patient in need thereof for a time sufficient to treat the cancer.

Further provided herein are methods for inhibiting growth or proliferation of cancer cells by administering a therapeutically effective amount of the CD79b x CD20 x CD3 trispecific antibody or trispecific binding fragment to inhibit the growth or proliferation of cancer cells.

Also provided herein are methods of redirecting a T cell to a CD79b and/or CD20-expressing cancer cell by administering a therapeutically effective amount of the CD79b x CD20 x CD3 trispecific antibody or trispecific binding fragment to redirect a T cell to a cancer.

The skilled person will understand that the methods of using the described CD79b x CD20 x CD3-multispecific antibodies and multispecific antigen-binding fragments thereof may be specified in medical use format, for example in the form of CD79b x CD20x CD3-multispecific antibodies and multispecific antigen-binding fragments for use in the treatment of disease as defined herein, in particular cancer. This skilled person will also understand that the methods of using the described CD79b x CD20 x CD3-multispecific antibodies and multispecific antigen-binding fragments thereof may be specified in so-called Swiss form, for example in the form of the use of CD79b x CD20 x CD3-multispecific antibodies and multispecific antigen-binding fragments for the manufacture of a medicament for the treatment of disease as defined herein, in particular cancer. This applies throughout the disclosure.

### CD79b x CD20 x CD3-Specific Antibody Kits

Described herein are kits including the disclosed CD79b x CD20 x CD3-multispecific antibodies. The described kits may be used to carry out the methods of using the CD79b x CD20 x CD3-multispecific antibodies provided herein, or other methods known to those skilled in the art. In some embodiments the described kits may include the antibodies described herein and reagents for use in treating a CD20 and/or CD79b-expressing cancer. Accordingly, the described kits may include one or more of the trispecific antibodies, or a trispecific antigen-binding fragment(s) thereof, described herein and a vessel for containing the antibody or fragment when not in use, and/or instructions for use of the antibody or fragment, the antibody or fragment affixed to a solid support, and/or detectably labeled forms of the antibody or fragment, as described herein.

### CD79b x CD3-Bispecific Antibodies

In some embodiments, provided herein are isolated CD79b x CD3 bispecific antibodies or antigen-binding fragments.

In some embodiments, an isolated CD79b x CD3 bispecific antibody, or a bispecific binding fragment thereof, comprises: a) a first antigen binding arm comprising a first heavy chain variable domain (VH1) and a first light chain variable domain (VL1); (b) a second antigen binding arm comprising a second heavy chain variable domain (VH2) and a second light chain variable domain (VL2), wherein the first antigen-binding arm binds to an epitope CD79b and the second antigen-binding arm binds to an epitope on CD3.

According to all aspects of the invention the CD79b x CD3 bispecific antibody, or a bispecific binding fragment thereof may bind to a conformational epitope of CD79 made up of residues 30-42 (SEDRYRNPKGSAC; SEQ ID NO: 253), 50-52 (PRF), 81-86 (EMENP; SEQ ID NO: 254), and 144-148 (GFSTL; SEQ ID NO: 255). The residue numbers are those of human CD79B (P40259).

According to all aspects of the invention the CD79b x CD20 x CD3 bispecific antibody, or a bispecific binding fragment thereof may bind to a conformational epitope of CD3 encompassing residues 54-58 (GSEIL; SEQ ID NO: 257), 74-75 (NI), and 100-105 (PRGSKP; SEQ ID NO: 258). The residue numbers are those of human CD3E (P07766).

In some embodiments, an isolated CD79b x CD3 bispecific antibody, or a bispecific binding fragment thereof, comprises: a) a first heavy chain portion (HC1); b) a light chain portion; c) a second heavy chain portion (HC2), wherein the HC1 and the LC form a first antigen-binding site that specifically binds a first antigen, the HC2 comprises a second antigen-binding site that specifically binds a second antigen, and the HC1 and HC2 each comprise a Fragment crystallizable (Fc) domain comprising a CH2-CH3 domain.

In some embodiments, the first antigen-binding arm of the CD79b x CD3 bispecific antibody, or a bispecific binding fragment thereof, comprises a first heavy chain portion (HC1) comprising the VH1, and a light chain portion (LC) comprising the VL1. The VH1 of the HC1 and the VL1 of the LC form a first antigen-binding domain that binds a first antigen. The second antigen-binding arm of the bispecific antibody or bispecific binding fragment thereof comprises a second heavy chain portion (HC2) comprising the VH2. The VH2 of the HC2 forms a second antigen-binding domain that binds a second antigen, and the HC1 and HC2 each optionally comprise a Fragment crystallizable (Fc) domain comprising a CH2-CH3 domain. In some embodiments, the first antigen is cluster of differentiation 79b (CD79b), and the second antigen is cluster of differentiation 3 (CD3). In some embodiments, the first antigen is cluster of differentiation 79b (CD79b), and the second antigen is cluster of differentiation 3 (CD3). Some aspects of the CD79b x CD3 bispecific antibody, or a bispecific binding fragment thereof, are further described in the Detail Description and Examples sections below.

In some embodiments, the CD79b-binding arm (or “CD79b-specific arm”) of the CD79b x CD3 bispecific antibody is derived from a CD79b antibody described herein (for example, from an antibody having the CDR sequences listed in Table 1a). In some embodiments, the CD79b-binding arm of the CD79b x CD3 bispecific antibody comprises any one VH domain and any one VL domain selected from Table 1b.

In some embodiments, the CD3-binding arm (or “CD3-specific arm”) of the CD79b x CD3 bispecific antibody is derived from a CD3 antibody described herein (for example, from an antibody having the CDR sequences listed in Table 3). In some embodiments, the CD3-binding arm of the CD79b x CD3 bispecific antibody comprises any one VH domain and any one VL domain selected from Table 3.

In some embodiments, the CD79b- or CD3-specific arm of the CD79b x CD3 bispecific antibodies or antigen-binding fragments are IgG, or derivatives thereof.

In one embodiment, the antibody comprises an Fc region with one or more of the following properties: (a) reduced effector function when compared to the parent Fc; (b) reduced affinity to FcγRI, FcγRIIa, FcγRIIb, FcγRIIIb and/or FcγRIIIa, (c) reduced affinity to FcγRI (d) reduced affinity to FcγRIIa (e) reduced affinity to FcγRIIb, (f) reduced affinity to FcγRIIIb or (g) reduced affinity to FcγRIIIa.

In some embodiments, the CD3-specific antibody or antigen-binding fragment from which the CD3-specific arm of the bispecific antibody is derived is IgG, or a derivative thereof. In some embodiments, the CD3-specific antibody or antigen-binding fragment from which the CD3-specific arm of the bispecific antibody is derived is IgG1, or a derivative thereof. In some embodiments, for example, the Fc region of the CD3-specific IgG1 antibody from which the CD3-binding arm is derived comprises L234A, L235A, and D265S substitutions in its Fc region. In some embodiments, the CD3-specific antibody or antigen-binding fragment from which the CD3-specific arm of the bispecific antibody is derived is IgG4, or a derivative thereof. In some embodiments, for example, the Fc region of the CD3-specific IgG4 antibody from which the CD3-binding arm is derived comprises S228P, L234A, L235A, F405L, and R409K substitutions in its Fc region. In some embodiments, the CD3-specific antibody or antigen-binding fragment from which the CD3-specific arm of the bispecific antibody is derived binds CD3ε on primary human T cells and/or primary cynomolgus T cells. In some embodiments, the CD3-specific antibody or antigen-binding fragment from which the CD3-specific arm of the bispecific antibody is derived activates primary human CD4+ T cells and/or primary cynomolgus CD4+ T cells.

In addition to the described CD79b x CD3 bispecific antibodies, also provided are polynucleotide sequences capable of encoding the described CD79b x CD3 bispecific antibodies. In some embodiments, an isolated synthetic polynucleotide encoding the one or more CDRs of the heavy chain variable domain and/or one or more CDRs of the light chain variable domain of each antigen-binding arm of the CD79b x CD3 bispecific antibody or bispecific binding fragment is provided. In some embodiments, an isolated synthetic polynucleotide encoding one or more heavy chain variable domains (such as the HC1 and/or the HC2) and/or one or more light chain variable domains of the CD79b x CD3 bispecific antibody or bispecific binding fragment is provided. In some embodiments, an isolated synthetic polynucleotide encoding one or more polypeptide chains of the first and/or second antigen-binding arms of the CD79b x CD3 bispecific antibody or bispecific binding fragment is provided. Vectors comprising the described polynucleotides are also provided, as are cells expressing the CD79b x CD3 bispecific antibodies provided herein. In another embodiment, an isolated cell expressing the bispecific antibody or bispecific binding fragment is provided. Also described are cells capable of expressing the disclosed vectors. These cells may be mammalian cells (such as 293 cells, 293F cells, CHO cells), insect cells (such as Sf7 cells), yeast cells, plant cells, or bacteria cells (such as E. coli). The described antibodies may also be produced by hybridoma cells. In some embodiments, methods for generating the CD79b x CD3 bispecific antibody or bispecific binding fragment by culturing cells is provided.

Further provided herein are pharmaceutical compositions comprising the CD79b x CD3 bispecific antibodies or antigen-binding fragments and a pharmaceutically acceptable carrier.

### Methods of Using CD79b x CD3 Bispecific Antibodies

Methods of using the described CD79b x CD3 bispecific antibodies and bispecific antigen-binding fragments thereof are also disclosed. For example, the CD79b x CD3 bispecific antibodies and bispecific antigen-binding fragments thereof may be useful in the treatment of a CD79b-expressing cancer in a subject in need thereof. In some embodiments, the CD79b-expressing cancer is a lymphoma, such as diffuse large B-cell lymphoma (DLBCL), mantle cell lymphoma (MCL), marginal zone lymphoma (MZL), follicular lymphoma (FL), chronic lymphocytic leukemia (CLL), or Waldenström macroglobulinemia (WM). In some embodiments, the CD79b-expressing cancer is a relapsed or refractory form of the lymphoma.

The described methods of treating CD79b-expressing cancer in a subject in need thereof include administering to the subject a therapeutically effective amount of a described CD79b x CD3 bispecific antibody or bispecific antigen-binding fragment thereof. In some embodiments, the subject is a mammal, preferably a human. In preferred embodiments are provided methods for treating a subject having cancer by administering a therapeutically effective amount of the CD79b x CD3 bispecific antibody or bispecific antigen-binding fragment to a patient in need thereof for a time sufficient to treat the cancer.

Further provided herein are methods for inhibiting growth or proliferation of cancer cells by administering a therapeutically effective amount of the CD79b x CD3 bispecific antibody or bispecific binding fragment to inhibit the growth or proliferation of cancer cells.

Also provided herein are methods of redirecting a T cell to a CD79b-expressing cancer cell by administering a therapeutically effective amount of the CD79b x CD3 bispecific antibody or bispecific binding fragment to redirect a T cell to a cancer.

### CD79b x CD3-Bispecific Antibody Kits

Described herein are kits including the disclosed CD79b x CD3 bispecific antibodies. The described kits may be used to carry out the methods of using the CD79b x CD3 bispecific antibodies provided herein, or other methods known to those skilled in the art. In some embodiments the described kits may include the antibodies described herein and reagents for use in treating a CD79b-expressing cancer. Accordingly, the described kits may include one or more of the bispecific antibodies, or a bispecific antigen-binding fragment(s) thereof, described herein and a vessel for containing the antibody or fragment when not in use, and/or instructions for use of the antibody or fragment, the antibody or fragment affixed to a solid support, and/or detectably labeled forms of the antibody or fragment, as described herein.

### CD79b-Specific Antibodies

Also provided herein are antibodies that bind to CD79b and antigen-binding fragments thereof. Also described are related polynucleotides capable of encoding the provided CD79b-specific antibodies and antigen-binding fragments, cells expressing the provided antibodies and antigen-binding fragments, as well as associated vectors and detectably labeled antibodies and antigen-binding fragments. In addition, methods of using the provided antibodies and antigen-binding fragments are described. For example, the CD79b-specific antibodies and antigen-binding fragments may be used to treat cancer (e.g., CD79b-expressing cancer); the CD79b-specific antibodies and antigen-binding fragments may be used to diagnose or monitor CD79b-expressing cancer progression, regression, or stability; to determine whether or not a patient should be treated for cancer; or to determine whether or not a subject is afflicted with CD79b-expressing cancer and thus may be amenable to treatment with a CD79b-specific anti-cancer therapeutic, such as the multispecific antibodies against CD79b and CD3 described herein. Some aspects of the CD79b-specific antibody, or an antigen-binding fragment, are further described in the Detail Description and Examples sections below.

According to all aspects of the invention the CD79b specific antibody or antigen-binding fragment may bind to a conformational epitope of CD79 made up of residues 30-42 (SEDRYRNPKGSAC; SEQ ID NO: 253), 50-52 (PRF), 81-86 (EMENP; SEQ ID NO: 254), and 144-148 (GFSTL; SEQ ID NO: 255). The residue numbers are those of human CD79B (P40259).

### Methods of Using CD79b-Specific Antibodies

Methods of using the described CD79b-specific antibodies or antigen-binding fragments are also disclosed. Particular antibodies for use in the methods discussed in this section include those with the set of CDRs described for antibodies in Table 1a. For example, these antibodies or antigen-binding fragments may be useful in treating cancer, by interfering with CD79b-receptor interactions or where the antibody is conjugated to a toxin, so targeting the toxin to the CD79b-expressing cancer. Further, these antibodies or antigen-binding fragments may be useful for detecting the presence of CD79b in a biological sample, such as blood or serum; for quantifying the amount of CD79b in a biological sample, such as blood or serum; for diagnosing CD79b-expressing cancer; determining a method of treating a subject afflicted with cancer; or monitoring the progression of CD79b-expressing cancer in a subject. In some embodiments, CD79b-expressing cancer may be a lymphoma, such as diffuse large B-cell lymphoma (DLBCL), mantle cell lymphoma (MCL), marginal zone lymphoma (MZL), follicular lymphoma (FL), chronic lymphocytic leukemia (CLL), or Waldenström macroglobulinemia (WM). In some embodiments, the CD79b-expressing cancer is a relapsed or refractory form of the lymphoma.

The described methods may be carried out before the subject receives treatment for CD79b-expressing cancer, such as treatment with a multispecific antibody against CD79b and CD3. Furthermore, the described methods may be carried out after the subject receives treatment for CD79b-expressing cancer, such as treatment with a multispecific antibody against CD79b and CD3 described herein.

The described methods of detecting CD79b in a biological sample include exposing the biological sample to one or more of the CD79b-specific antibodies or antigen-binding fragments described herein.

The described methods of diagnosing CD79b-expressing cancer in a subject also involve exposing the biological sample to one or more of the CD79b-specific antibodies or antigen-binding fragments described herein; however, the methods also include quantifying the amount of CD79b present in the sample; comparing the amount of CD79b present in the sample to a known standard or reference sample; and determining whether the subject's CD79b levels fall within the levels of CD79b associated with cancer.

Also described herein are methods of monitoring CD79b-expressing cancer in a subject. The described methods include exposing the biological sample to one or more of the CD79b-specific antibodies or antigen-binding fragments described herein; quantifying the amount of CD79b present in the sample that is bound by the antibody, or antigen-binding fragment thereof; comparing the amount of CD79b present in the sample to either a known standard or reference sample or the amount of CD79b in a similar sample previously obtained from the subject; and determining whether the subject's CD79b levels are indicative of cancer progression, regression or stable disease based on the difference in the amount of CD79b in the compared samples.

The samples obtained, or derived from, subjects are biological samples such as urine, blood, serum, plasma, saliva, ascites, circulating cells, circulating tumor cells, cells that are not tissue associated, tissues, surgically resected tumor tissue, biopsies, fine needle aspiration samples, or histological preparations.

The described CD79b-specific antibodies or antigen-binding fragments may be labeled for use with the described methods, or other methods known to those skilled in the art. For example, the antibodies described herein, or antigen-binding fragments thereof, may be labeled with a radiolabel, a fluorescent label, an epitope tag, biotin, a chromophore label, an ECL label, an enzyme, ruthenium, ¹¹¹In-DOTA, ¹¹¹In-diethylenetriaminepentaacetic acid (DTPA), horseradish peroxidase, alkaline phosphatase and beta-galactosidase, or poly-histidine or similar such labels known in the art.

### CD79b-Specific Antibody Kits

Described herein are kits including the disclosed CD79b-specific antibodies or antigen-binding fragments thereof. The described kits may be used to carry out the methods of using the CD79b-specific antibodies or antigen-binding fragments provided herein, or other methods known to those skilled in the art. In some embodiments the described kits may include the antibodies or antigen-binding fragments described herein and reagents for use in detecting the presence of CD79b in a biological sample.

Accordingly, the described kits may include one or more of the antibodies, or an antigen-binding fragment(s) thereof, described herein and a vessel for containing the antibody or fragment when not in use, instructions for use of the antibody or fragment, the antibody or fragment affixed to a solid support, and/or detectably labeled forms of the antibody or fragment, as described herein.

### BRIEF DESCRIPTION OF THE DRAWINGS

FIG. 1. Depiction of an exemplary CD79b x CD20 x CD3 trispecific antibody. FIG. 1 discloses SEQ ID NO: 221.

FIGS. 2A-2D. Binding affinities of selected CD79bxCD3 bsAbs in the HBL-1 cell line (FIG. 2A); the OCI-LY10 cell line (FIG. 2B); the Carnaval cell line (FIG. 2C); and the WILL-2 cell line (FIG. 2D). Circles correspond to the 79C3B646 bsAb; triangles correspond to the 79C3B651 bsAb; and diamonds correspond to the 79C3B601 bsAb.

FIGS. 3A-3D. Binding affinities of selected CD79bxCD20xCD3 trispecific antibodies in the HBL-1 cell line (FIG. 3A); the OCI-LY10 cell line (FIG. 3B); the Carnaval cell line (FIG. 3C); and the WILL-2 cell line (FIG. 3D). Solid circles correspond to the 79C3B646 bsAb control; solid triangles correspond to the 79C3B651 bsAb control; and solid diamonds correspond to the 79C3B601 bsAb control. Open triangles correspond to trispecific antibody C923B38; open diamonds correspond to trispecific antibody C923B74; asterisks correspond to trispecific antibody C923B9; and X corresponds to control null trispecific antibody C923B98.

FIGS. 4A-4I. Binding kinetics of selected CD79bxCD3 bsAbs on DLBCL cell lines. Binding kinetics of the three selected bsAbs in HBL-1 cells at 300 nm (FIG. 4A). Binding kinetics of the three selected bsAbs in HBL-1 cells at 60 nm (FIG. 4B). Binding kinetics of the three selected bsAbs in HBL-1 cells at 12 nm (FIG. 4C). Binding kinetics of the three selected bsAbs in Carnaval cells at 300 nm (FIG. 4D). Binding kinetics of the three selected bsAbs in Carnaval cells at 60 nm (FIG. 4E). Binding kinetics of the three selected bsAbs in Carnaval cells at 12 nm (FIG. 4F). Binding kinetics of the three selected bsAbs in OCI-LY10 cells at 300 nm (FIG. 4G). Binding kinetics of the three selected bsAbs in OCI-LY10 cells at 60 nm (FIG. 4H). Binding kinetics of the three selected bsAbs in OCI-LY10 cells at 12 nm (FIG. 4I). Inverted triangles correspond to the 79C3B646 bsAb; diamonds correspond to the 79C3B651 bsAb; and squares correspond to the 79C3B601 bsAb.

FIGS. 5A-5I. Binding kinetics of selected CD79bxCD20xCD3 trispecific antibodies on DLBCL cell lines. Binding kinetics of the selected antibodies in HBL-1 cells at 300 nm (FIG. 5A). Binding kinetics of the selected antibodies in HBL-1 cells at 60 nm (FIG. 5B). Binding kinetics of the selected antibodies in HBL-1 cells at 12 nm (FIG. 5C). Binding kinetics of the selected antibodies in Carnaval cells at 300 nm (FIG. 5D). Binding kinetics of the selected antibodies in Carnaval cells at 60 nm (FIG. 5E). Binding kinetics of the selected antibodies in Carnaval cells at 12 nm (FIG. 5F). Binding kinetics of the selected antibodies in OCI-LY10 cells at 300 nm (FIG. 5G). Binding kinetics of the selected antibodies in OCI-LY10 cells at 60 nm (FIG. 5H). Binding kinetics of the selected antibodies in OCI-LY10 cells at 12 nm (FIG. 5I). Inverted triangles correspond to the 79C3B646 bsAb control; diamonds correspond to the 79C3B651 bsAb control; and squares correspond to the 79C3B601 bsAb control. Triangles correspond to trispecific antibody C923B38; circles correspond to trispecific antibody C923B74; squares correspond to trispecific antibody C923B99; and asterisks correspond to control null trispecific antibody C923B98.

FIGS. 6A-6D. Primary pan T-cell binding of CD79bxCD20xCD3 trispecific antibodies and CD79bxCD3 bispecific antibodies. Binding kinetics of the selected antibodies in pan T-cell donor line D221837 (FIG. 6A). Binding kinetics of the selected antibodies in pan T-cell donor line D329312 (FIG. 6B). Binding kinetics of the selected antibodies in pan T-cell donor line D329335 (FIG. 6C). Binding kinetics of the selected antibodies in pan T-cell donor line D160115 (FIG. 6D). Circles correspond to the 79C3B651 bsAb; squares correspond to the 79C3B646 bsAb; triangles correspond to the trispecific antibody C923B38; inverted triangles correspond to the trispecific antibody C923B99; diamonds correspond to the trispecific antibody C923B74.

FIGS. 7A-7B. T cell cytotoxicity of CD79bxCD20xCD3 trispecific antibodies and CD79bxCD3 bispecific antibodies. Cytotoxicity of the selected antibodies in the HEL T-cell line (FIG. 7A). Cytotoxicity of the selected antibodies in the K562 T-cell line (FIG. 7B). Shaded circles correspond to the trispecific antibody C923B74; clear circles correspond to the trispecific antibody C923B99; triangles correspond to the trispecific antibody C923B38; inverted triangles correspond to the 79C3B646 bsAb; diamonds correspond to 79C3B651 bsAb; black squares correspond to the 79C3B601 bsAb; and white squares correspond to C923B98 bsAb.

FIGS. 8A-8C. CD79bxCD20xCD3 trispecific construct mediated B cell cytotoxicity and T cell activation. Cytotoxicity in B cells (FIG. 8A); CD4+ T-cells (FIG. 8B) and CD8⁺ T-cells are shown for the lead antibodies.

FIGS. 9A-9C. CD79b and CD20 structure. (FIG. 9A) B cell receptor (BCR) and CD79b structure (Burger et al 2018 (19)). (FIG. 9B) CD79b long and short isoform schematic representation. aa, amino acid. (FIG. 9C) CD20 structure (modified from Klein et al 2013 (20)). FIG. 9C discloses SEQ ID NOS 264 and 265, respectively, in order of appearance.

FIG. 10. CD79b and CD20 B-cell marker expression in cancer (mRNA levels). AML, acute myeloid leukemia; CD, cluster of differentiation; CML, chronic myelogenous leukemia; DLBCL, diffused large B cell lymphoma; FL, follicular lymphoma; MCL, mantle cell lymphoma; MDS, myelodysplastic syndrome; MGUS, monoclonal gammopathy of undetermined significance; MM, multiple myeloma. InforMe database was used to quantitate the relative mRNA levels of CD22, MS4A1 (CD20), CD79a, and CD79b across hematological malignancies using the GeneLogic Heme Plus 2.0 data set. mRNA levels are expressed as z score, which describes the expression of a gene relative to the average across all of the samples. The darker the color the lower the z-score (corresponding to lower expression), the lighter the color the higher z-score (corresponding to higher gene expression). Based on this heat map, high expression of CD79b and CD20 was detected in DLBCL, FL, and MCL, but not in AML, MDS, MGUS, CML, MM.

FIGS. 11A-11C. B-cell marker expression in FFPE tumor sections derived from B NHL patients (IHC) and cell lines. CD20, CD79b, CD79a, CD22 IHC staining performed on samples from B-NHL patient at (FIG. 11A) diagnosis or (FIG. 11B) relapse. B-NHL, B-cell non-Hodgkin lymphoma; CD, cluster of differentiation; FFPE, formalin-fixed, paraffin-embedded; GC, germinal center; IHC, immunohistochemistry; MALTL, mucosa-associated lymphoid tissue lymphoma; NHL, non-Hodgkin lymphoma; NON-GC, non-germinal center; R-CHOP, rituximab-cyclophosphamide-hydroxydaunorubicin-oncovin-prednisone/prednisolone regimen; R/R, relapsed/refractory. FIG. 11C shows CD20 and CD79b IHC expression in cell lines vs B-NHL samples. Scale 3, 2, 1, 0 represent high, medium, low, no detectable expression, respectively.

FIGS. 12A-12B. Exemplary binding profiles of C923B169 on CARNAVAL and OCI Ly10 cell lines after 1 hour 37° C. incubation. (FIG. 12A) CARNAVAL cell binding of C923B169. (FIG. 12B) OCI-Ly10 cell binding of C923B169. MFI, mean fluorescence intensity.

FIGS. 13A-13B. Exemplary kinetic binding profiles of C923B169 on CARNAVAL and OCI Ly10 cell lines throughout 48 hours 37° C. incubation. (FIG. 13A) CARNAVAL kinetic binding of C923B169. (FIG. 13B) OCI Ly10 kinetic binding of C923B169. MFI, mean fluorescence intensity.

FIGS. 14A-14C. Binding profiles of C923B169 of primary T cells from 3 donors after 1 hour 37° C. incubation. (FIG. 14A) T cell binding: D327645. (FIG. 14B) T cell binding: D198013. (FIG. 14C) T cell binding: D221837.

FIGS. 15A-15C. Therapeutic efficacy (in vitro cytotoxicity assay) comparing CD79bxCD20xCD3 (C923B169) to single-antigen NullxCD20xCD3 engagement. Cancer cell killing was assessed over time using Incucyte technology after coculture of pan CD3 T cells with K562 cancer cells expressing CD79b or CD20 or CD79b/CD20 at 5:1 E:T ratio. AUC calculation of (FIG. 15A) K562 CD79b⁺, (FIG. 15B) K562 CD20⁺, and (FIG. 15C) K562 CD79b⁺/CD20⁺ cancer cell growth inhibition over time as determined by mKate2⁺/GFP⁺ area per well was done over 6-day time frame. After normalizing the AUC to the untreated control (target with effector), antibody concentrations were plotted against the AUC values as a dose response. EC₅₀ values were generated using GraphPad PRISM v.9. AUC, area under the curve; EC50, 50% effective concentration; E:T ratio, effector-to-target ratio.

FIGS. 16A-16H. C923B169-induced T cell-mediated cytotoxicity of different cancer cell lines. The percentage of cell line cytotoxicity was determined by FACS quantitation (Y-axis). CFSE-labeled cell lines were combined with CD3⁺ pan T cells at a 5:1 effector-to-target ratio for either 48 or 72 hours with increasing concentrations (X-axis) of C923B169 or NullxNullxCD3. Values are averages of 5 to 6 individual T-cell donors. All cell lines are CD79b⁺/CD20⁺ except SU-DHL1 which is CD79b⁻/CD20⁻. Graphing of data was done in GraphPad Prism 9. Data from independent experiments were pooled and represented as mean±SEM. CFSE, carboxyfluorescein succinimidyl ester; FACS, fluorescence activated cell sorting; SEM, standard error of the mean. FIGS. 16G-16H demonstrate that C923B169 exhibits no cytotoxicity in CD79b⁻/CD20⁻ cells.

FIGS. 17A-17F. C923B169-induced T-cell activation in the presence of different cancer cell lines. The percentage of CD8 T-cell activation was determined by FACS quantitation (Y-axis) of CD25⁺ cells. CFSE-labeled cell lines were combined with CD3⁺ pan T cells at a 5:1 effector-to-target ratio for either 48 or 72 hours with increasing concentrations (X-axis) of C923B169 or NullxNullxCD3. Values are averages of 5 to 6 individual T-cell donors. All cell lines are CD79b⁺/CD20⁺ except SU-DHL1, which is CD79b⁻/CD20⁻. As SU-DHL1 secretes IL-2 and thus induces CD25 on T cells in the coculture system, for assessing % of CD25 on CD8 T cells, the gate was set relative to untreated wells with SU-DHL1 and T cells. Graphing of data was done in GraphPad Prism 9. Data from independent experiments were pooled and represented as mean±SEM. CFSE, carboxyfluorescein succinimidyl ester; FACS, fluorescence activated cell sorting; IL, interleukin; SEM, standard error of the mean.

FIG. 18. C923B169-induced inflammatory cytokines in vitro in the presence of CARNAVAL cells at a 5:1 E:T ratio at 48 and 72 hours. CFSE-labeled CARNAVAL cells were added to CD3 T cells at 5:1 E:T ratio for 48 or 72 hours with increasing concentrations of C923B169. Supernatant was analyzed for inflammatory cytokines using MSD Proinflammatory kit (MSD K15049D). Values are averages of 6 individual healthy donors. Graphing of data was done in GraphPad Prism 9. Data from independent experiments were pooled and represented as mean±SEM. Ab, antibody; CFSE, carboxyfluorescein succinimidyl ester; E:T ratio, effector-to-target ratio; IFN, interferon; IL, interleukin; MSD, Meso Scale Discovery; SEM, standard error of the mean; TNF, tumor necrosis factor.

FIG. 19. Autologous B cell depletion assay showing cytotoxicity of C923B169 on primary target B cells.

FIGS. 20A-20D. Autologous B-cell depletion assay showing T-cell activation profiles with C923B169 treatment. (FIG. 20A) Activated CD4+ T cells by C923B169. (FIG. 20B) Activated CD8+ T cells by C923B169. (FIG. 20C) Secreted IFN γ levels by C923B169. (FIG. 20D) Secreted IL 10 levels by C923B169.

FIG. 21. C923B169-induced antagonism of IL 10 secretion. C923B169 was added at a range of concentrations to activated B cell DLBCL cell line (OCI Ly10) for 24 hours to assess the effect on IL 10 secretion. IL 10 levels were normalized to untreated control cells and expressed as percent.

FIGS. 22A-22B. Antitumor efficacy of C923B169 in subcutaneous CARNAVAL xenografts (FIG. 22A) and OCI Ly10 xenograft (FIG. 22B) in T cell-humanized NSG mice. T cell-humanized (T cell injection indicated by grey arrow) NOD scid gamma or NOD.Cg Prkdc^{scid} Il2rg^{tm1Wjl}/SzJ (NSG) mice injected SC with (FIG. 22A) CARNAVAL tumors or (FIG. 22B) OCI Ly10 tumors were dosed intraperitoneally (IP) with C923B169 at 1 and 5 mg/kg for CARNAVAL model or at 3 and 10 mg/kg for OCI Ly10 model (dosing indicated by black arrows). Tumor volume was measured twice weekly and results presented as the mean tumor volume±SEM for each group. Data graphically represented for each group with >66% of mice remaining. PBS, phosphate-buffered saline.

FIG. 23. C923B169-induced inflammatory cytokines in vitro in the presence of CARNAVAL cells at a 1:1 E:T ratio at 48 and 72 hours. CFSE-labeled CARNAVAL cells were added to CD3 T cells at 1:1 E:T ratio for 48 or 72 hours with increasing concentrations of C923B169. Supernatant was analyzed for inflammatory cytokines using MSD Proinflammatory kit (MSD K15049D). Values are averages of 6 individual healthy donors. Graphing of data was done in GraphPad Prism 9. Data from independent experiments were pooled and represented as mean±SEM. Ab, antibody; CFSE, carboxyfluorescein succinimidyl ester; E:T ratio, effector-to-target ratio; IFN, interferon; IL, interleukin; MSD, Meso Scale Discovery; SEM, standard error of the mean; TNF, tumor necrosis factor.

FIG. 24. C923B169-induced inflammatory cytokines in vitro in the presence of OCI-Ly10 cells at a 5:1 E:T ratio at 48 and 72 hours. CFSE-labeled OCI-Ly10 cells were added to CD3 T cells at 5:1 E:T ratio for 48 or 72 hours with increasing concentrations of C923B169. Supernatant was analyzed for inflammatory cytokines using MSD Proinflammatory kit (MSD K15049D). Values are averages of 6 individual healthy donors. Graphing of data was done in GraphPad Prism 9. Data from independent experiments were pooled and represented as mean±SEM. OCI-Ly10 cells downstream of NF-κB secrete IL-10. As C923B169 mediates CD79b antagonistic effect on OCI-Ly10 cancer cell (see Example 9), yet in the cancer cell and T-cell coculture system it mediates IL-10 cytokines secretion from T cells. The effect observed on IL-10 cytokine in this coculture system is a balance between the antagonistic effect of C923B169 on OCI-Ly10 cancer cells versus effect of C923B169 on T-cell-mediated cytokine secretion. Ab, antibody; CD, cluster of differentiation; CFSE, carboxyfluorescein succinimidyl ester; E:T ratio, effector-to-target ratio; IFN, interferon; IL, interleukin; MSD, Meso Scale Discovery; NF-κB, nuclear factor kappa-light-chain-enhancer of activated B cells; SEM, standard error of the mean; TNF, tumor necrosis factor.

FIG. 25. C923B169-induced inflammatory cytokines in vitro in the presence of OCI-Ly10 cells at a 1:1 E:T ratio at 48 and 72 hours. CFSE-labeled OCI-Ly10 cells were added to CD3 T cells at 1:1 E:T ratio for 48 or 72 hours with increasing concentrations of C923B169. Supernatant was analyzed for inflammatory cytokines using MSD Proinflammatory kit (MSD K15049D). Values are averages of 6 individual healthy donors. Graphing of data was done in GraphPad Prism 9. Data from independent experiments were pooled and represented as mean±SEM. OCI-Ly10 cells downstream of NF-κB secrete IL-10. As C923B169 mediates CD79b antagonistic effect on OCI-Ly10 cancer cell (see Example 9), yet in the cancer cell and T-cell coculture system it mediates IL-10 cytokines secretion from T cells. The effect observed on IL-10 cytokine in this coculture system is a balance between the antagonistic effect of C923B169 on OCI-Ly10 cancer cells versus effect of C923B169 on T-cell-mediated cytokine secretion. Ab, antibody; CFSE, carboxyfluorescein succinimidyl ester; E:T ratio, effector-to-target ratio; IFN, interferon; IL, interleukin; MSD, Meso Scale Discovery; NF-κB, nuclear factor kappa-light-chain-enhancer of activated B cells; SEM, standard error of the mean; TNF, tumor necrosis factor.

FIGS. 26A-26B. Binding profiles of C923B169, C923B168, and mosunetuzumab after 1-hour 37° C. incubation with CARNAVAL (FIG. 26A) and OCI-Ly10 (FIG. 26B) cell lines. MFI, mean fluorescence intensity.

FIGS. 27A-27D. Binding kinetic profiles of C923B169, C923B168, and mosunetuzumab after 48-hour 37° C. incubation with CARNAVAL and OCI-Ly10 cell lines. MFI, mean fluorescence intensity.

FIGS. 28A-28C. Primary T-cell binding profiles of C923B169, C923B168, and mosunetuzumab in Donor D327645 (FIG. 28A), Donor D198013 (FIG. 28B), and Donor D221837 (FIG. 28C). MFI, mean fluorescence intensity.

FIG. 29. Autologous B-cell depletion assay showing cytotoxicity of C923B169, C923B168, and mosunetuzumab against B cells.

FIGS. 30A-30D. Autologous B-cell depletion assay showing T-cell activation profiles of C923B169, C923B168, and mosunetuzumab.

FIG. 31. CD79b and CD20 expression profile in peripheral blood. CD79b and CD20 receptor density results in several immune cell populations were evaluated in whole blood derived from 3 different healthy donors. CD79b expression was determined by flow cytometry using a PE-labeled commercial antibody (human CD79B-PE clone #SN8, BD Bioscience #335833), CD20 expression was determined by flow cytometry using a PE-labeled commercial antibody (human CD20-PE clone #2H7, BD Bioscience #555623), and receptor counts were calculated using the Quantum Simply cellular kit (Bangslabs #815). NKT, natural killer T cell; mDC, myeloid dendritic cell; pDC, plasmacytoid dendritic cell; PE, phycoerythrin.

FIG. 32. CD79b expression profile in normal tissues. Photomicrographs of CD79b immunohistochemistry (IHC)-stained core biopsies from an formalin-fixed, paraffin-embedded (FFPE) human normal tissue microarray (TMA). Strong positive staining of mononuclear cells was present (CD79b antibody: CST rabbit mAb clone D7V2F; 0.15 μg/mL).

FIG. 33. Mean (standard deviation) serum concentration-time profiles following repeated dosing of 1 or 5 mg/kg C923B169 in CARNAVAL-bearing xenograft mice (n=10/group).

FIG. 34. Preliminary C923B169 PK results in cyno monkeys.

FIG. 35. Mean (SD) serum C923B169 concentration-time profiles following a single intravenous (IV) or subcutaneous (SC) dose of 1 mg/kg of C923B169 in male minipigs. Data points with concentrations below the lowest quantifiable concentration are not shown in the graph.

FIG. 36. Individual serum C923B169 concentration-time profiles following a single IV dose of 1 mg/kg of C923B169 in male minipigs (Group 3). Data points with concentrations below the lowest quantifiable concentration are not shown in the graph.

FIG. 37. Individual serum C923B169 concentration-time profiles following single SC dose of 1 mg/kg of C923B169 in male minipigs (Group 4). Data points with concentrations below the lowest quantifiable concentration are not shown in the graph.

### DETAILED DESCRIPTION OF ILLUSTRATIVE EMBODIMENTS

B-cell malignancies encompasses a variety of cancers including CLL, MCL, DLBCL, FL, and MZL and represent a significant unmet medical need. After the successes of small-molecule inhibitors targeting the BCR or apoptotic pathways, T-cell redirection approaches have been selected as one of the main areas of research and development. The off-the-shelf preparation of T-cell-engagers provides a new therapeutic option for a broad patient population, from frontline to heavily pretreated patients, including those who progressed after chimeric antigen receptor (CAR)-T cell therapy (28).

Non-Hodgkin lymphoma (NHL) is a heterogeneous group of malignancies, from B- or T-cell origin, accounting for about 4% of all malignancies in the US (29). Approximately 80% of NHLs are derived from the B-cell lineage and express B-cell differentiation antigens, including CD19, CD20, CD22, and CD79b. These surface antigens represent pivotal targets for current standard-of-care treatment. However, despite the improvements in the available therapies, B-NHLs carry a uniformly poor prognosis with 30% to 50% of DLBCL patients relapsing after rituximab-cyclophosphamide-hydroxydaunorubicin-oncovin-prednisone/prednisolone regimen (R-CHOP) therapy, and only 40% of patients with relapsed and refractory (R/R) disease achieving long-term complete remission upon CD19 CAR-T therapy (30). Emerging clinical trial data with CD20xCD3 bispecific antibodies appear to demonstrate that T-cell redirection may address the urgent unmet need of this growing population whose disease no longer responds to standard chemo- or immunotherapies.

Both CD79b and CD20 are well-validated therapeutic targets expressed in most B-cell malignancies; therefore, they serve as good surface antigens for use in therapeutic modalities. Dual antigen recognition on B cells with a trispecific T-cell redirecting antibody as described herein has the potential to enhance tumor binding through avidity effects, maximize tumor eradication in the presence of a heterogeneous cell population, and prevent tumor antigen escape from occurring, which has been observed with CD19- and CD20-targeting therapies (31-34).

DLBCL is the most prevalent subset of aggressive B-NHLs accounting for approximately 30% to 58% of all new cases of NHL diagnosed annually worldwide. Patient's shares in the R/R setting is dominated in the US and in Europe by the rituximab-ifosfamide-carboplatin-etoposide (R-ICE), rituximab-(dose-adjusted) etoposide-prednisolone-oncovin-cyclophosphamide-hydroxydaunorubicin (R-[DA]-EPOCH), and rituximab-dexamethasone-high-dose ara-C cytarabine-platinol (R-DHAP) regimens followed by rituximab-etoposide-solu-medrol-high-dose ara-C cytarabine-platinol (R-ESHAP) and bendamustine-rituximab (BR) regimens.

CAR-T therapies have been recently approved in this setting and are expected to represent an important uptake over the next decade. The entry of 3 anti-CD19 CAR-T therapies, ie, Yescarta, Kymriah, and Breyanzi into the R/R DLBCL space marks the entry of a new drug class for the treatment of NHL. All 3 therapies target chemorefractory patients, including those who experienced disease progression following allogeneic stem-cell transplantation (ASCT). The high costs associated with CAR-T therapies, the logistics involved in preparing the constructs, and the acute toxicity associated with treatment will impede their uptake and alternative treatment options are needed.

Monoclonal antibodies are dominating the R/R FL setting in terms of patient shares. The 2017 R/R patient share leader in the US was BR, followed by R-CHOP. Lenalidomide+rituximab, the so-called R² regimen, has been approved in 2019 and is recommended by National Comprehensive Cancer Network (NCCN) guidelines as treatment option for this patient population. Zydelig (idelalisib), a phosphoinositide 3-kinase (PI3K) inhibitor, is also approved for patients who have received 2 prior lines. Ukoniq (umbralisib) and Tazverik (tasemetostat) have been approved in 2020 for R/R patients after 2 or more lines of therapy. Unmet need is especially high for early R/R patients, who have limited effective treatment options.

The multispecific antibodies and multispecific antigen-binding fragments described herein address these and other related needs.

### Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of up to ±10% from the specified value, as such variations are appropriate to perform the disclosed methods. Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

“Isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An “isolated” antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to CD79b is substantially free of antibodies that specifically bind antigens other than CD79b). An isolated antibody that specifically binds to an epitope, isoform or variant of CD79b may, however, have cross-reactivity to other related antigens, for instance from other species (such as CD79b species homologs).

“Polynucleotide,” synonymously referred to as “nucleic acid molecule,” “nucleotides” or “nucleic acids,” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications may be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

“Synthetic nucleic acid sequence,” “synthetic polynucleotide,” “synthetic oligonucleotide,” “synthetic DNA.” or “synthetic RNA” as used herein refers to a nucleic acid sequence, a polynucleotide, an oligonucleotide, DNA, or RNA that differs from one found in nature by having a different sequence than one found in nature or a chemical modification not found in nature. The definition of synthetic nucleic acid includes but is not limited to a DNA sequence created using biotechnology tools. Such tools include but are not limited to recombinant DNA technology, chemical synthesis. or directed use of nucleases (so called “genome editing” or “gene optimizing” technologies). The meaning of “substantially the same” can differ depending on the context in which the term is used. Because of the natural sequence variation likely to exist among heavy and light chains and the genes encoding them, one would expect to find some level of variation within the amino acid sequences or the genes encoding the antibodies or antigen-binding fragments described herein, with little or no impact on their unique binding properties (e.g., specificity and affinity). Such an expectation is due in part to the degeneracy of the genetic code, as well as to the evolutionary success of conservative amino acid sequence variations, which do not appreciably alter the nature of the encoded protein. Accordingly, in the context of nucleic acid sequences, “substantially the same” means at least 65% identity between two or more sequences. Preferably, the term refers to at least 70% identity between two or more sequences, more preferably at least 75% identity, more preferably at least 80% identity, more preferably at least 85% identity, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, and more preferably at least 99% or greater identity. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The percent identity between two nucleotide or amino acid sequences may e.g. be determined using the algorithm of E. Meyers and W. Miller, Comput. Appl. Biosci 4, 11-17 (1988) which has been incorporated into the ALIGN program (version 2.0), using a PAM120 weight residue table, a gap length penalty of 12 and a gap penalty of 4. In addition, the percent identity between two amino acid sequences may be determined using the Needleman and Wunsch, J. Mol. Biol. 48, 444-453 (1970) algorithm.

The degree of variation that may occur within the amino acid sequence of a protein without having a substantial effect on protein function is much lower than that of a nucleic acid sequence, since the same degeneracy principles do not apply to amino acid sequences. Accordingly, in the context of an antibody or antigen-binding fragment, “substantially the same” means antibodies or antigen-binding fragments having 90%, 91%, 92%, 93%, 94%, 95%, 96%, 97%, 98%, or 99% identity to the antibodies or antigen-binding fragments described. Other embodiments include antibodies, or antigen-binding fragments, that have framework, scaffold, or other non-binding regions that do not share significant identity with the antibodies and antigen-binding fragments described herein, but do incorporate one or more CDRs or other sequences needed to confer binding that are 90%, 91%, 92%, 93%, 94%, 95%, 96%, 97%, 98%, or 99% identical to such sequences described herein.

A “clone” is a population of cells derived from a single cell or common ancestor by mitosis. A “cell line” is a clone of a primary cell that is capable of stable growth in vitro for many generations. In some examples provided herein, cells are transformed by transfecting the cells with DNA.

The terms “express” and “produce” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications. The expression or production of an antibody or antigen-binding fragment thereof may be within the cytoplasm of the cell, or into the extracellular milieu such as the growth medium of a cell culture.

The terms “treating” or “treatment” refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

An “effective amount” or “therapeutically effective amount” refers to an amount effective, at dosages and for periods of time necessary, to achieve a desired therapeutic result. A therapeutically effective amount of an antibody described herein may vary according to factors such as the disease state, age, sex, and weight of the individual, and the ability of the antibody to elicit a desired response in the individual. A therapeutically effective amount is also one in which any toxic or detrimental effects of the antibody or antibody portion are outweighed by the therapeutically beneficial effects.

“Antibody” refers to all isotypes of immunoglobulins (IgG, IgA, IgE, IgM, IgD, and IgY) including various monomeric, polymeric and chimeric forms, unless otherwise specified. Specifically encompassed by the term “antibody” are polyclonal antibodies, monoclonal antibodies (mAbs), and antibody-like polypeptides, such as chimeric antibodies and humanized antibodies.

The term “antigen-binding arm” refers to a portion of an antibody that includes an antigen-binding domain or an antigen-binding site that binds to an antigen (e.g., CD79b, CD20, or CD3), and optionally includes one or more other antibody regions (e.g., Fc domain). An antigen-binding arm is an example of an “antigen-binding region”. “Antigen-binding fragments” are any proteinaceous structure that may exhibit binding affinity for a particular antigen. Similarly, “bispecific binding fragment” or “trispecific-binding fragment” are any proteinaceous structure that may exhibit binding affinity for two or three antigens, respectively. As used herein, the term “antigen-binding fragment”, “bispecific binding fragment”, or “trispecific-binding fragment” preferably refers to a fragment of the antigen-binding arm containing an antigen-binding domain. Binding fragments include those provided by any known technique, such as enzymatic cleavage, peptide synthesis, and recombinant techniques. Some binding fragments are composed of portions of intact antibodies that retain antigen-binding specificity of the parent antibody molecule. For example, binding fragments may comprise at least one variable region (either a heavy chain or light chain variable region) or one or more CDRs of an antibody known to bind a particular antigen. Examples of suitable binding fragments include, without limitation diabodies and single-chain molecules as well as Fab, F(ab′)2, Fc, Fabc, and Fv molecules, single chain (Sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782, bivalent fragments comprising two Fab fragments linked by a disulfide bridge at the hinge region, a Fd fragment consisting essentially of the VH and CH1 domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al; Trends Biotechnol. 2003 Nov.; 21(11):484-90); camelid or nanobodies (Revets et al; Expert Opin Biol Ther. 2005 January; 5(1):111-24); an isolated complementarity determining region (CDR), and the like. All antibody isotypes may be used to produce binding fragments. Additionally, binding fragments may include non-antibody proteinaceous frameworks that may successfully incorporate polypeptide segments in an orientation that confers affinity for a given antigen of interest, such as protein scaffolds. Antigen-binding fragments, bispecific binding fragments, or trispecific-binding fragments may be recombinantly produced or produced by enzymatic or chemical cleavage of intact antibodies. The phrase “an antibody or antigen-binding fragment thereof”, “a bispecific antibody or bispecific-binding fragment thereof”, or “a trispecific antibody or trispecific binding fragment thereof” may be used to denote that a given antigen-binding fragment incorporates one or more amino acid segments of the antibody referred to in the phrase.

The term “antigen-binding domain” refers to the proteinaceous structure of an antigen-binding arm that exhibits binding affinity for a particular antigen. This proteinaceous structure is mediated by the complementarity determining regions (CDRs) of the antigen-binding domain.

The terms “CDR”, and its plural “CDRs”, refer to a complementarity determining region (CDR) of which three make up the binding character of a light chain variable region (CDRL1, CDRL2 and CDRL3) and three make up the binding character of a heavy chain variable domain (CDRH1, CDRH2 and CDRH3). CDRs contribute to the functional activity of an antibody molecule and are separated by amino acid sequences that comprise scaffolding or framework regions. The exact definitional CDR boundaries and lengths are subject to different classification and numbering systems. CDRs may therefore be referred to by Kabat, Chothia, contact or any other boundary definitions. The CDRs described herein are referred to by the AbM definition. Despite differing boundaries, each of these systems has some degree of overlap in what constitutes the so called “hypervariable regions” within the variable sequences. CDR definitions according to these systems may therefore differ in length and boundary areas with respect to the adjacent framework region. See for example Kabat et al., Sequences of Proteins of Immunological Interest, 5th ed. NIH Publication No. 91-3242 (1991); Chothia et al., “Canonical Structures For the Hypervariable Regions of Immunoglobulins,” J. Mol. Biol. 196:901 (1987); and MacCallum et al., “Antibody-Antigen Interactions: Contact Analysis and Binding Site Topography,” J. Mol. Biol. 262:732 (1996)), each of which is hereby incorporated by reference in its entirety.

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three dimensional structures, despite high amino acid sequence variability in most parts of the loops (Chothia et al., “Canonical Structures For the Hypervariable Regions of Immunoglobulins,” J. Mol. Biol. 196:901 (1987); Chothia et al., “Conformations of Immunoglobulin Hypervariable Regions,” I 342:877 (1989); Martin and Thornton, “Structural Families in Loops of Homologous Proteins: Automatic Classification, Modelling and Application to Antibodies,” J. Mol. Biol. 263:800 (1996), each of which is incorporated by reference in its entirety). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

As used herein the term “Fc” refers to the fragment crystallizable domain of an antibody, which comprises two constant heavy chain (CH) regions, CH2 and CH3. Herein, the amino acid residues of the Fc region are typically numbered according to the EU numbering scheme (Edelman, G. M. et al., Proc. Natl. Acad. USA, 63, 78-85 (1969). PMID: 5257969). These residues can be readily assigned according to alternative numbering schemes such as IMGT and Kabat (Kabat, E. A. et al., Sequences of proteins of immunological interest. 5th Edition—US Department of Health and Human Services, NIH publication n° 91-3242, pp 662,680,689 (1991)) numbering as would be readily appreciated by one skilled in the art. For example, L234 according to EU numbering may also be represented as L247 according to Kabat.

The term “polypeptide” is used interchangeably with the term “protein” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to either natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein.

“Specifically binds” or “binds specifically” or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a K_{d} of less than about 1×10⁻⁸ M, as measured by a surface plasmon resonance assay or a cell-binding assay. Phrases such as “[antigen]-specific” antibody (e.g., CD79b-specific antibody) are meant to convey that the recited antibody specifically binds the recited antigen. Wherever the term “binds” is used herein it is intended that this encompasses “specifically binds” and the terms may be interchanged as desired.

As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

A “vector” is a replicon, such as plasmid, phage, cosmid, or virus in which another nucleic acid segment may be operably inserted so as to bring about the replication or expression of the segment.

As used herein, the term “host cell” can be any type of cell, e.g., a primary cell, a cell in culture, or a cell from a cell line. In some embodiments, the host cell is ex vivo or in vitro. In some embodiments, the host cell is not a human totipotent or human pluripotent stem cell. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome. The terms “expression” and “production” are used synonymously herein, and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term “subject” refers to human and non-human animals, including all vertebrates, e.g., mammals and non-mammals, such as non-human primates, mice, rabbits, sheep, dogs, cats, horses, cows, chickens, amphibians, and reptiles. In many embodiments of the described methods, the subject is a human.

The term “redirect” or “redirecting” as used herein refers to the ability of the described multispecific antibody (e.g., a CD79b x CD20 x CD3 antibody, a CD79b x CD3 antibody) to traffic the activity of T cells effectively, from its inherent cognate specificity toward reactivity against CD79b and/or CD20-expressing cells. The term “sample” as used herein refers to a collection of similar fluids, cells, or tissues (e.g., surgically resected tumor tissue, biopsies, including fine needle aspiration), isolated from a subject, as well as fluids, cells, or tissues present within a subject. In some embodiments the sample is a biological fluid. Biological fluids are typically liquids at physiological temperatures and may include naturally occurring fluids present in, withdrawn from, expressed or otherwise extracted from a subject or biological source. Certain biological fluids derive from particular tissues, organs or localized regions and certain other biological fluids may be more globally or systemically situated in a subject or biological source. Examples of biological fluids include blood, serum and serosal fluids, plasma, lymph, urine, saliva, cystic fluid, tear drops, feces, sputum, mucosal secretions of the secretory tissues and organs, vaginal secretions, ascites fluids such as those associated with non-solid tumors, fluids of the pleural, pericardial, peritoneal, abdominal and other body cavities, fluids collected by bronchial lavage and the like. Biological fluids may also include liquid solutions contacted with a subject or biological source, for example, cell and organ culture medium including cell or organ conditioned medium, lavage fluids and the like. The term “sample,” as used herein, encompasses materials removed from a subject or materials present in a subject. The relevant aspects of the invention may be performed ex vivo or in vitro based on isolated samples as required.

A “known standard” may be a solution having a known amount or concentration of CD79b and/or CD20, where the solution may be a naturally occurring solution, such as a sample from a patient known to have early, moderate, late, progressive, or static cancer, or the solution may be a synthetic solution such as buffered water having a known amount of CD79b and/or CD20 diluted therein. The known standards, described herein may include CD79b and/or CD20 isolated from a subject, recombinant or purified CD79b and/or CD20 protein, or a value of CD79b and/or CD20 concentration associated with a disease condition.

“Cluster of Differentiation CD79B protein” or “CD79b” refers to a B-cell antigen receptor (BCR) signaling component Igβ. The amino acid sequences of the various isoforms are retrievable from GenBank accession numbers AAH32651.1, EAW94232.1, AAH02975.2, NP_000617.1, and NP_001035022.1. The amino acid sequence of the full length CD79b sequence is shown below. The sequence includes the extracellular domain (residues 29-159) and the cytoplasmic domain (residues 181-229).

(SEQ ID NO: 252)
MARLALSPVPSHWMVALLLLLSAEPVPAARSEDRYRNPKGSACSRIWQS
PRFIARKRGFTVKMHCYMNSASGNVSWLWKQEMDENPQQLKLEKGRMEE
SQNESLATLTIQGIRFEDNGIYFCQQKCNNTSEVYQGCGTELRVMGFST
LAQLKQRNTLKDGIIMIQTLLIILFIIVPIFLLLDKDDSKAGMEEDHTY
EGLDIDQTATYEDIVTLRTGEVKWSVGEHPGQE

“Cluster of Differentiation 20” or “CD20” refers to an antigenic determinant known to be detectable on B cells. Human CD20 is also called membrane-spanning 4-domains, subfamily A, member 1 (MS4A1). The human and murine amino acid and nucleic acid sequences can be found in a public database, such as GenBank, UniProt and Swiss-Prot. For example, the amino acid sequence of human CD20 can be found at Accession Nos. NP_690605.1 and NP_068769.2, and the nucleic acid sequence encoding transcript variants 1 and 3 of the human CD20 can be found at Accession No. NM_152866.2 and NM_021950.3, respectively.

The term “CD3” refers to the human CD3 protein multi-subunit complex. The CD3 protein multi-subunit complex is composed to 6 distinctive polypeptide chains. These include a CD3γ chain (SwissProt P09693), a CD3δ chain (SwissProt P04234), two CD3ε chains (SwissProt P07766), and one CD3ξ chain homodimer (SwissProt 20963), and which is associated with the T cell receptor α and β chain. The term “CD3” includes any CD3 variant, isoform and species homolog which is naturally expressed by cells (including T cells) or can be expressed on cells transfected with genes or cDNA encoding those polypeptides, unless noted.

A “CD79b x CD20 x CD3 antibody” is a multispecific antibody, optionally a trispecific antibody, which comprises three different antigen-binding arms, one of which binds to the antigen CD79b, one of which binds to the antigen CD20, and one of which binds to CD3. A “CD79b x CD3 antibody” is a multispecific antibody, optionally a bispecific antibody, which comprises two different antigen-binding arms, one of which binds to the antigen CD79b and one of which binds to CD3. A “CD20 x CD3 antibody” is a multispecific antibody, optionally a bispecific antibody, which comprises two different antigen-binding arms, one of which binds to the antigen CD20 and one of which binds to CD3. The term “multispecific antibody” is used herein in the broadest sense and specifically covers an antibody that has polyepitopic specificity. Multispecific antibodies include, but are not limited to, an antibody comprising a heavy chain variable domain (VH) and a light chain variable domain (VL), where the VHVL unit has polyepitopic specificity, antibodies having two or more V_{L} and V_{H} domains where each VHVL unit binds to a different epitope, antibodies having two or more single variable domains with each single variable domain binding to a different epitope, full length antibodies, and antibodies comprising one or more antibody fragments as well as antibodies comprising antibody fragments that have been linked covalently or non-covalently.

A multispecific antibody can be a bispecific antibody, a trispecific antibody, diabody, or similar molecule (see for instance PNAS USA 90(14), 6444-8 (1993) for a description of diabodies). The bispecific antibodies, trispecific antibodies, diabodies, and the like, provided herein may bind any suitable target in addition to a portion of CD79b or CD20. The term “bispecific antibody” is to be understood as an antibody having two different antigen-binding arms defined by different antibody sequences. The term “trispecific antibody” is to be understood as an antibody having three different antigen-binding arms defined by different antibody sequences. This can be understood as different target binding but includes as well binding to different epitopes in one target.

A “reference sample” is a sample that may be compared against another sample, such as a test sample, to allow for characterization of the compared sample. The reference sample will have some characterized property that serves as the basis for comparison with the test sample. For instance, a reference sample may be used as a benchmark for CD79b or CD20 levels that are indicative of a subject having cancer. The reference sample does not necessarily have to be analyzed in parallel with the test sample, thus in some instances the reference sample may be a numerical value or range previously determined to characterize a given condition, such as CD79b or CD20 levels that are indicative of cancer in a subject. The term also includes samples used for comparative purposes that are known to be associated with a physiologic state or disease condition, such as CD79b- or CD20-expressing cancer, but that have an unknown amount of CD79b or CD20.

“Relapsed” refers to the return of a disease or the signs and symptoms of a disease after a period of improvement after prior treatment with a therapeutic.

“Refractory” refers to a disease that does not respond to a treatment. A refractory disease can be resistant to a treatment before or at the beginning of the treatment, or a refractory disease can become resistant during a treatment.

The term “progression,” as used in the context of progression of CD79b and/or CD20-expressing cancer, includes the change of a cancer from a less severe to a more severe state. This may include an increase in the number or severity of tumors, the degree of metastasis, the speed with which the cancer is growing or spreading, and the like. For example, “the progression of colon cancer” includes the progression of such a cancer from a less severe to a more severe state, such as the progression from stage I to stage II, from stage II to stage III, etc.

The term “regression,” as used in the context of regression of CD79b and/or CD20-expressing cancer, includes the change of a cancer from a more severe to a less severe state. This could include a decrease in the number or severity of tumors, the degree of metastasis, the speed with which the cancer is growing or spreading, and the like. For example, “the regression of colon cancer” includes the regression of such a cancer from a more severe to a less severe state, such as the progression from stage III to stage II, from stage II to stage I, etc.

The term “stable” as used in the context of stable CD79b and/or CD20-expressing cancer, is intended to describe a disease condition that is not, or has not, changed significantly enough over a clinically relevant period of time to be considered a progressing cancer or a regressing cancer.

The embodiments described herein are not limited to particular methods, reagents, compounds, compositions or biological systems, which can, of course, vary.

### Multispecific Antibodies

Multispecific antibodies that bind to CD79b, CD20, and/or CD3, and multispecific binding fragments thereof are provided herein. Such antibodies or antibody fragments may allow for more specific targeting to particular subsets of cells as compared to antibodies targeting only one or two of these targets.

### Trispecific Antibodies

In some embodiments, provided herein are trispecific antibodies that bind to CD79b, CD20, and CD3, and trispecific binding fragments thereof. This can be achieved by, for example, making a molecule which comprises a first antigen-binding arm binding to CD79b, a second antigen-binding arm binding to CD3, and a third antigen-binding arm binding to the CD20. The antigen-binding arms can take any form that allows specific recognition of the target, for example the binding arm may be or may include a heavy chain variable domain, an Fv (combination of a heavy chain variable domain and a light chain variable domain), an single-chain Fv (scFv), an Fab, a binding domain based on a fibronectin type III domain (such as from fibronectin, or based on a consensus of the type III domains from fibronectin, or from tenascin or based on a consensus of the type III domains from tenascin, such as the Centyrin molecules from Janssen Biotech, Inc., see e.g. WO2010/051274 and WO2010/093627). In certain embodiments, the trispecific antibody comprises three antigen binding arms. In some embodiments, the trispecific antibody is comprised of an antibody (e.g. in IgG format) to which an additional antigen-binding arm, e.g. in the form of a single chain variable fragment, is fused, e.g. to the N or C-terminus of one of the heavy or one of the light chains of the antibody.

Accordingly, trispecific molecules comprising three different antigen-binding arms which bind CD79b, CD20, and CD3, respectively, are provided.

In some embodiments, the CD79b x CD20 x CD3-multispecific antibody comprises:
(a) a first antigen-binding arm comprising a first heavy chain variable domain (VH1) and a first light chain variable domain (VL1);
(b) a second antigen-binding arm comprising a second heavy chain variable domain (VH2) and a second light chain variable domain (VL2);
(c) a third antigen-binding arm comprising a third heavy chain variable domain (VH3) and a third light chain variable domain (VL3),
wherein the first antigen-binding arm binds to an epitope on CD79b, the second antigen-binding arm binds to an epitope on CD3, and the third antigen-binding arm binds to an epitope on CD20.

In some embodiments, the VH1 comprises an amino acid sequence selected from SEQ ID NO: 35, SEQ ID NO: 39, SEQ ID NO: 43, SEQ ID NO: 45, SEQ ID NO: 49, SEQ ID NO: 55, SEQ ID NO: 59, SEQ ID NO: 63, SEQ ID NO: 67, and SEQ ID NO: 71.

In some embodiments, the VL1 comprises an amino acid sequence selected from SEQ ID NO: 37, SEQ ID NO: 41, SEQ ID NO: 47, SEQ ID NO: 51, SEQ ID NO: 53, SEQ ID NO: 57, SEQ ID NO: 61, SEQ ID NO: 65, SEQ ID NO: 69, and SEQ ID NO: 73.

In some embodiments, the VH2 comprises an amino acid sequence selected from SEQ ID NO: 97, SEQ ID NO: 101, SEQ ID NO: 103, SEQ ID NO: 105, SEQ ID NO: 107, SEQ ID NO: 196, and SEQ ID NO: 206.

In some embodiments, the VL2 comprises an amino acid sequence selected from SEQ ID NO: 99, SEQ ID NO: 109, SEQ ID NO: 201, and SEQ ID NO: 211.

In some embodiments, the VH3 comprises an amino acid sequence selected from SEQ ID NO: 126, SEQ ID NO: 130, SEQ ID NO: 134, and SEQ ID NO: 138.

In some embodiments, the VL3 comprises an amino acid sequence selected from SEQ ID NO: 128, SEQ ID NO: 132, SEQ ID NO: 136, and SEQ ID NO: 140.

In some embodiments, the VH1 and VL1 of the antigen-binding arm that binds to CD79b epitope are present in a diabody, a Fab, Fab′, a F(ab′)2, a Fv, a scFv, a Fd, a disulfide stabilized Fv fragment (dsFv), or a disulfide stabilized diabody (ds diabody).

In some embodiments, the VH2 and VL2 of the antigen-binding arm that binds to CD3 epitope are present in a diabody, a Fab, Fab′, a F(ab′)2, a Fv, a scFv, a Fd, a disulfide stabilized Fv fragment (dsFv), or a disulfide stabilized diabody (ds diabody).

In some embodiments, the VH3 and VL3 of the antigen-binding arm that binds to CD20 epitope are present in a diabody, a Fab, Fab′, a F(ab′)2, a Fv, a scFv, a Fd, a disulfide stabilized Fv fragment (dsFv), or a disulfide stabilized diabody (ds diabody).

In some embodiments, the first antigen-binding arm of the CD79b x CD20 x CD3 multispecific antibody comprises a first heavy chain portion (HC1) comprising the VH1, and a light chain (LC) comprising the VL1, where the VH1 and VL1 pair to form a first antigen-binding domain that binds a first antigen. In some embodiments, the HC1 comprises, from N to C-terminus, the VH1, a first heavy chain constant domain (CH1), and a first Fc domain. In some embodiments, the VH1 and CH1 of the HC1 together with the LC form a fragment antigen binding (Fab) domain.

In some embodiments, the VH1 of the first antigen-binding arm is coupled to the VH3 of the third antigen-binding arm via the first Fc domain. In some embodiments, the first Fc domain of the first antigen-binding arm is coupled, via a first linker (L1), to the third antigen-binding arm, thereby forming a coupled first and third antigen-binding arm. The coupled first and third antigen-binding arms may comprise, from N to C-terminus, the VH1, the CH1 domain, and the Fc domain of the first antigen-binding arm, the first linker, and the third antigen-binding arm. In some embodiments, the third antigen-binding arm is a single-chain variable fragment (scFv) formed from the VH3 and VL3 of the third-antigen-binding arm.

In some embodiments, the second antigen-binding arm of the CD79b x CD20 x CD3 multispecific antibody comprises a second heavy chain portion (HC2) comprising the second heavy chain variable domain (VH2) which forms a second antigen-binding domain that binds a second antigen. In some embodiments, the second binding arm comprises from N to C-terminus, a single-chain variable fragment (scFv) formed from the VH2 and VL2, and a second Fc domain.

In some embodiments, the VH2 of the second antigen-binding arm is coupled to the VH3 of the third antigen-binding arm via the second Fc domain. In some embodiments, the second Fc domain of the second antigen-binding arm is coupled, via a linker, to the third antigen-binding arm, thereby forming a coupled second and third antigen-binding arm. The coupled second and third antigen-binding arms may comprise, from N to C-terminus, the second antigen-binding domain, the second Fc domain, the first linker, and the third antigen-binding arm. In some embodiments, the third antigen-binding arm is a single-chain variable fragment (scFv) formed from the VH3 and VL3 of the third-antigen-binding arm.

In preferred embodiments, the CD79b x CD20 x CD3-multispecific antibody is a trispecific antibody comprising a CD79b-specific binding arm comprising the first heavy chain portion (HC1) with the VH1 and the light chain portion (LC) with the VL1. The VH1 and VL1 pair to form a first antigen-binding domain that binds CD79b. The second antigen-binding arm of the trispecific antibody comprises the second heavy chain portion (HC2) with the VH2 that forms the second antigen-binding domain that binds the second antigen. The HC1 of the CD79b-specific binding arm or the HC2 of the second antigen-binding arm is coupled to the third antigen-binding arm comprising the VH3 domain, which forms the third antigen-binding domain that binds the third antigen. In some embodiments, the second antigen is CD20, and the third antigen is CD3. In some embodiments, the second antigen is CD3, and the third antigen is CD20.

In one embodiment, the CD79b x CD20 x CD3-multispecific antibody is a trispecific antibody comprising a CD79b-specific binding arm comprising the HC1 with the VH1 and the LC with the VL1. The VH1 and VL1 pair to form the first CD79b-specific antigen-binding domain that binds CD79b. The second antigen-binding arm comprises the VH2 and VL2 that form the second antigen-binding domain that binds CD3. The third antigen-binding arm is coupled to the second antigen-binding arm and comprises the VH3 and VL3 that form the third antigen-binding domain that binds CD20.

In one embodiment, the CD79b x CD20 x CD3-multispecific antibody is a trispecific antibody comprising a CD79b-specific binding arm comprising the HC1 with the VH1 and the LC with the VL1. The VH1 and VL1 pair to form the first CD79b-specific antigen-binding domain that binds CD79b. The second antigen-binding arm comprises the VH2 and VL2 that form the second antigen-binding domain that binds CD20. The third antigen-binding arm is coupled to the second antigen-binding arm and comprises the VH3 and VL3 that form the third antigen-binding domain that binds CD3.

In one embodiment, the CD79b x CD20 x CD3-multispecific antibody is a trispecific antibody comprising a CD79b-specific binding arm comprising the HC1 with the VH1 and the LC with the VL1. The VH1 and VL1 pair to form the first CD79b-specific antigen-binding domain that binds CD79b. The second antigen-binding arm comprises the VH2 and VL2 that form the second antigen-binding domain that binds CD3. The third antigen-binding arm is coupled to the first CD79b-specific antigen-binding arm and comprises the VH3 and VL3 that form the third antigen-binding domain that binds CD20.

In one embodiment, the CD79b x CD20 x CD3-multispecific antibody is a trispecific antibody comprising a CD79b-specific binding arm comprising the HC1 with the VH1 and the LC with the VL1. The VH1 and VL1 pair to form the first CD79b-specific antigen-binding domain that binds CD79b. The second antigen-binding arm comprises the VH2 and VL2 that form the second antigen-binding domain that binds CD20. The third antigen-binding arm is coupled to the first CD79b-specific antigen-binding arm and comprises the VH3 and VL3 that form the third antigen-binding domain that binds CD3.

In some embodiments, the HC1 with the VH1 and LC with VL1 of the first antigen-binding arm form an antigen-binding fragment (Fab) comprising the first antigen-binding domain. In some embodiments, the VH2 and VL2 of the second antigen-binding arm form a single-chain variable fragment (scFv) comprising the second antigen-binding domain. In some embodiments, the VH3 and VL3 of the third antigen-binding arm form a single-chain variable fragment (scFv) comprising the third antigen-binding domain.

In one embodiment, the CD79b-binding arm comprises an antigen-binding fragment (Fab), the CD3-binding arm comprises a single-chain variable fragment (scFv), and the CD20-binding arm comprises a single-chain variable fragment (scFv).

In some embodiments, the CD79b binding arm of the trispecific antibody comprises the HC1 and the LC. The HC1 may comprise constant heavy chain regions (CH1, CH2, and CH3) and the VH1. The LC may comprise the VL1. The VH1 and VL1 combine to form the CD79b antigen binding domain.

In some embodiments, CD3 binding arm of the trispecific antibody comprises the HC2. The HC2 may comprise constant heavy chain regions (CH2 and CH3), and a single-chain variable fragment (scFv) attached at the N-terminus of the CH2 region, wherein the scFv comprises the CD3 antigen binding domain.

In some embodiments, the trispecific antibody further comprises CD20 antigen-binding arm attached to the C-terminus of the CH3 region of the CD3 binding arm to form a CD3/CD20 binding arm. In some embodiments, the CD20 antigen-binding arm comprises a second single-chain variable fragment (scFv). In some embodiments, the CD3/CD20 arm may have the structure: scFV containing the CD3 binding domain, CH2 and CH3 regions, scFV containing the CD20 binding domain.

### Bispecific Antibodies

In some embodiments, provided herein are bispecific antibodies that bind to CD79b and CD3, and bispecific binding fragments thereof. This can be achieved by, for example, making a molecule which comprises a first antigen-binding arm binding to CD79b, and a second antigen-binding arm binding to CD3. The antigen-binding arms can take any form that allows specific recognition of the target, for example the binding region may be or may include a heavy chain variable domain, an Fv (combination of a heavy chain variable domain and a light chain variable domain), an single-chain Fv (scFv), an Fab, a binding domain based on a fibronectin type III domain (such as from fibronectin, or based on a consensus of the type III domains from fibronectin, or from tenascin or based on a consensus of the type III domains from tenascin, such as the Centyrin molecules from Janssen Biotech, Inc., see e.g. WO2010/051274 and WO2010/093627). Accordingly, bispecific molecules comprising three different antigen-binding arms which bind CD79b and CD3, respectively, are provided.

In some embodiments, the CD79b x CD3 bispecific antibody comprises:
(a) a first antigen binding arm comprising a first heavy chain variable domain (VH1) and a first light chain variable domain (VL1); and
(b) a second antigen-binding arm comprising a first heavy chain variable domain (VH2) and a first light chain variable domain (VL2),
wherein the first antigen-binding arm binds to an epitope on CD79b, and the second antigen-binding arm binds to an epitope on CD3.

In some embodiments, the VH1 and VL1 of the antigen binding arm that binds to CD79b epitope are present in a diabody, a Fab, Fab′, a F(ab′)2, a Fv, a scFv, a Fd, a disulfide stabilized Fv fragment (dsFv), or a disulfide stabilized diabody (ds diabody).

In some embodiments, the VH2 and VL2 of the antigen binding arm that binds to CD3 epitope are present in a diabody, a Fab, Fab′, a F(ab′)2, a Fv, a scFv, a Fd, a disulfide stabilized Fv fragment (dsFv), or a disulfide stabilized diabody (ds diabody).

In some embodiments, the first antigen-binding arm comprises a first heavy chain portion (HC1) comprising the VH1, and a light chain (LC) comprising the VL1. In some embodiments, the HC1 comprises, from N to C-terminus, the VH1, a first heavy chain constant domain (CH1), and a first Fc domain. In some embodiments, the VH1 and CH1 of the HC1 together with the LC form a fragment antigen binding (Fab) domain.

In some embodiments, the second antigen-binding arm comprises, from N to C-terminus, a single-chain variable fragment (scFv) formed from the VH2 and VL2, and a second Fc domain.

In some embodiments, the first binding arm of the CD79b x CD3-multispecific antibody comprises the HC1 comprising the VH1 and the LC comprising the VL1. The VH1 and VL1 pair to form a first antigen-binding domain that binds the first antigen of the bispecific antibody. The second antigen-binding arm of the bispecific antibody comprises a second heavy chain portion (HC2) comprising the VH2 that forms the second antigen-binding domain that specifically binds the second antigen. The HC1 and HC2 may each comprise a Fragment crystallizable (Fc) domain comprising a CH2-CH3 domain.

In some embodiments, the CD79b x CD3-bispecific antibody comprises a CD79b-specific binding arm comprising the HC1 with the VH1 and the LC with the VL1. The VH1 and VL1 pair to form the first antigen-binding domain that binds CD79b. The second antigen-binding arm of the bispecific antibody comprises the HC2 with the VH2 forming the second antigen-binding domain that binds CD3.

In some embodiments, the CD79b x CD3-bispecific antibody comprises a CD3-specific binding arm comprising the HC1 with the VH1 and the LC with the VL1. The VH1 and VL1 pair to form the first antigen-binding domain that binds CD3. The second antigen-binding are of the bispecific antibody comprises the HC2 with the VH2 forming a second antigen-binding domain that binds CD79b.

In some embodiments, the HC1 with the VH1 and LC with the VL1 form an antigen-binding fragment (Fab) comprising the first antigen-binding domain. In some embodiments, the HC2 with the VH2, together with VL2, forms a single-chain variable fragment (scFv) comprising the second antigen-binding domain.

In one embodiment, the CD79b-binding arm comprises an antigen-binding fragment (Fab), and the CD3-binding arm comprises a single-chain variable fragment (scFv).

In one embodiment, the CD3-binding arm comprises an antigen-binding fragment (Fab), and the CD79b-binding arm comprises a single-chain variable fragment (scFv).

In some embodiments, the multispecific antibodies (e.g., trispecific antibodies, bispecific antibodies) of the invention include antibodies having a full length antibody structure. “Full length antibody” as used herein refers to an antibody having two full length antibody heavy chains and two full length antibody light chains. A full length antibody heavy chain (HC) includes heavy chain variable and constant domains VH, CH1, CH2, and CH3. A full length antibody light chain (LC) includes light chain variable and constant domains VL and CL. The full length antibody may be lacking the C-terminal lysine (K) in either one or both heavy chains. The term “Fab-arm” or “half molecule” refers to one heavy chain-light chain pair that specifically binds an antigen. In some embodiments, one of the antigen-binding domains is a non-antibody based binding domain, e.g. a binding domain of based on a fibronectin type 3 domain, e.g. Centyrin.

### CD79b-Binding Arm

The multispecific antibodies (e.g., trispecific or bispecific antibodies) described herein comprise an antigen-binding arm specific for CD79b. In some embodiments, the CD79b-binding arm binds human CD79b. In some embodiments, the CD79b-binding arm binds human CD79b and cynomolgus monkey CD79b. In some embodiments, the CD79b-binding arm binds human CD79b but not to cynomolgus monkey CD79b. In some embodiments, the CD79b-binding arm binds bind to an epitope including one or more residues from the CD79b extracellular domain (ECD). In some embodiments, the CD79b-binding arm binds to one or more residues of a polypeptide having the amino acid sequence of SEQ ID NO: 252. In some embodiments, the CD79b-binding arm binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b. Such CD79b-binding arms may bind to CD79b with an affinity of 5×10⁻⁷M or less, such as 1×10⁻⁷M or less, 5×10⁻⁸M or less, 1×10⁻⁸M or less, 5×10⁻⁹M or less, 1×10⁻⁹M or less, or 5×10⁻¹⁰M or less. In one embodiment, the CD79b-binding arm binds to the CD79b with an affinity of about 1×10⁻¹¹M to 1×10⁻⁹M. In one embodiment, the CD79b-binding arm binds to the CD79b with an affinity of about 1×10⁻¹¹M, about 2×10⁻¹¹M, about 3×10⁻¹¹M, about 4×10⁻¹¹M, about 5×10⁻¹¹M, about 6×10⁻¹¹M, about 7×10⁻¹¹M, about 8×10⁻¹¹M, about 9×10⁻¹¹M, 1×10⁻¹⁰M, about 2×10⁻¹⁰M, about 3×10⁻¹⁰M, about 4×10⁻¹⁰M, about 5×10⁻¹⁰M, about 6×10⁻¹⁰M, about 7×10⁻¹⁰M, about 8×10⁻¹⁰M, about 9×10⁻¹⁰M or about 1×10⁻⁹M.

Table 1a and Table 1b provide a summary of CDR (as defined by AbM) and VH and VL sequences of some exemplary CD79b-specific antibodies described herein:

TABLE la
CDR sequences of exemplary mAbs generated against human CD79b

| ID | HC-CDR1 | HC-CDR2 | HC-CDR3 | LC-CDR1 | LC-CDR2 | LC-CDR3 |
|---|---|---|---|---|---|---|
| CD9B374 | GASISSFYW | RISPSGKTN | GEYSGTYS | RSSESLLDS | TLSYRAS | MQRMEFPL |
| | S (SEQ ID | (SEQ ID NO: | YSFDV (SEQ | EDGNTYLD | (SEQ ID NO: | T (SEQ ID |
| | NO: 1) | 2) | ID NO: 3) | (SEQ ID NO: | 5) | NO: 6) |
| | | | | 4) | | |
| CD9B330 | GDSVSNNS | RTYYRSKW | VDIAFDY | SGSSSNIGN | NDDLLPS | AAWDDSLN |
| | ATWN (SEQ | YND (SEQ | (SEQ ID NO: | HGVN (SEQ | (SEQ ID NO: | GVV (SEQ |
| | ID NO: 13) | ID NO: 8) | 9) | ID NO: 10) | 11) | ID NO: 12) |
| CD9B330- | GDSVSSNS | RTYYRSKW | VDIAFDY | SGSSSNIGN | NDDLLPS | AAWDDSLN |
| N31S | ATWN (SEQ | YND (SEQ | (SEQ ID NO: | HGVN (SEQ | (SEQ ID NO: | GVV (SEQ |
| | ID NO: 7) | ID NO: 8) | 9) | ID NO: 10) | 11) | ID NO: 12) |
| CD9B643 | GVSISNYY | RISPSGRTN | GEYSGTYS | RSSQSLFDS | TLSYRAS | MQRMEFPL |
| | WS (SEQ ID | (SEQ ID NO: | YSFDI (SEQ | DDGNTYLD | (SEQ ID NO: | T (SEQ ID |
| | NO: 14) | 15) | ID NO: 16) | (SEQ ID NO: | 5) | NO: 6) |
| | | | | 17) | | |
| CD9B321 | GDSVSSNS | RTYYRSKW | VNTTFDY | SGSSSNIGK | SDDLLSS | AAWDDSLN |
| | AAWN (SEQ | YND (SEQ | (SEQ ID NO: | NAVS (SEQ | (SEQ ID NO: | GVV (SEQ |
| | ID NO: 18) | ID NO: 8) | 19) | ID NO: 20) | 21) | ID NO: 12) |
| CD9B324 | GDSVSNNS | RTYYRSKW | VDIAFDY | SGSSSNIGN | NDDLLPS | AAWDDSLN |
| | ATWN (SEQ | YND (SEQ | (SEQ ID NO: | HGVN (SEQ | (SEQ ID NO: | GVV (SEQ |
| | ID NO: 13) | ID NO: 8) | 9) | ID NO: 10) | 11) | ID NO: 12) |
| CD9B332 | GDSVSNNS | RTYYRSKW | VDIAFDY | SGSSSNIGN | NDDLLPS | AAWDDSLN |
| | ATWN (SEQ | YND (SEQ | (SEQ ID NO: | HGVN (SEQ | (SEQ ID NO: | GVV (SEQ |
| | ID NO: 13) | ID NO: 8) | 9) | ID NO: 10) | 11) | ID NO: 12) |
| CD9B369 | GASISSYY | RISNTGRTN | GEYSGTFSY | RSSLSLLDS | TLSYRAS | MQRMEFPL |
| | WS (SEQ ID | (SEQ ID NO: | GFDI (SEQ | DDGKIYLD | (SEQ ID NO: | T (SEQ ID |
| | NO: 22) | 23) | ID NO: 24) | (SEQ ID NO: | 5) | NO: 6) |
| | | | | 25) | | |
| CD9B381 | GASISSYY | RIYSNGNIN | GEYSGDFS | RSSQSLLDS | TLSYRAS | MQRIEFPLT |
| | WS (SEQ ID | (SEQ ID NO: | YSFDI (SEQ | DDGNTYLD | (SEQ ID NO: | (SEQ ID NO: |
| | NO: 22) | 26) | ID NO: 27) | (SEQ ID NO: | 5) | 29) |
| | | | | 28) | | |
| CD9B389 | GVSISNYY | RISPSGRTN | GEYSGTYS | RSSQSLFDS | TLSYRAS | MQRMEFPL |
| | WS (SEQ ID | (SEQ ID NO: | YSFDI (SEQ | DDGNTYLD | (SEQ ID NO: | T (SEQ ID |
| | NO: 14) | 15) | ID NO: 16) | (SEQ ID NO: | 5) | NO: 6) |
| | | | | 17) | | |
| CD9B390 | GGSISNYY | RIFYSGKTN | GEYSGEYS | RSSQSLLDS | TLSYRAS | MQRMEFPL |
| | WS (SEQ ID | (SEQ ID NO: | YSFDI (SEQ | DDGNTYVD | (SEQ ID NO: | T (SEQ ID |
| | NO: 30) | 31) | ID NO: 32) | (SEQ ID NO: | 5) | NO: 6) |
| | | | | 33) | | |

TABLE 1b
VH and VL sequences of exemplary mAbs generated
against human CD79b

| | VH amino | VH | VL amino acid | VL DNA |
|---|---|---|---|---|
| | acid | DNA | sequence | sequence |
| | sequence SEQ | SEQ | SEQ ID | SEQ |
| ID | ID NO | ID NO | NO | ID NO |
| CD9B374 | 35 | 36 | 37 | 38 or 213 |
| CD9B330 | 39 | 40 | 41 | 42 |
| CD9B330-N31S | 43 | 44 | 41 | 34 |
| CD9B643 | 45 | 46 | 47 | 48 or 214 |
| CD9B321 | 49 | 50 | 51 | 52 |
| CD9B324 | 39 | 40 | 53 | 54 |
| CD9B332 | 55 | 56 | 57 | 58 |
| CD9B369 | 59 | 60 | 61 | 62 |
| CD9B381 | 63 | 64 | 65 | 66 |
| CD9B389 | 67 | 68 | 69 | 70 |
| CD9B390 | 71 | 72 | 73 | 74 |

In some embodiments, the CD79b-binding arm comprises a heavy chain variable domain comprising a CDR1, a CDR2, and a CDR3 of any one of the antibodies described in Table 1a. In some embodiments, the CD79b-binding arm comprises a light chain variable region comprising a CDR1, a CDR2, and a CDR3 of any one of the antibodies described in Table 1a. In some embodiments, the CD79b-binding arm comprises a heavy chain variable domain comprising a CDR1, a CDR2, and a CDR3 of any one of the antibodies described in Table 1a and a light chain variable region comprising a CDR1, a CDR2, and a CDR3 of any one of the antibodies described in Table 1a. In some embodiments, the CD79b-binding arm competes for binding to CD79b with an antibody or antigen-binding comprising a heavy chain comprising a CDR1, a CDR2, and a CDR3 of any one of the antibodies described in Table 1a and a light chain comprising a CDR1, a CDR2, and a CDR3 of any one of the antibodies described in Table 1a.

In some embodiments, the CD79b-binding arm comprises a heavy chain variable domain of any one of the antibodies described in Table 1b. In some embodiments, the CD79b-binding arm comprises a light chain variable region of any one of the antibodies described in Table 1b. In some embodiments, the CD79b-binding arm comprises a heavy chain variable domain of any one of the antibodies described in Table 1b and a light chain variable region of any one of the antibodies described in Table 1b. In some embodiments, the CD79b-binding arm competes for binding to CD79b with an antibody or antigen-binding comprising a heavy chain variable domain of any one of the antibodies described in Table 1b and a light chain variable domain of any one of the antibodies described in Table 1b.

In some embodiments, the CD79b-binding arm comprises a heavy chain comprising a CDR1, a CDR2, and a CDR3 of any one of the antibodies described in Table 1a. In some embodiments, the CD79b-binding arm comprises a light chain comprising a CDR1, a CDR2, and a CDR3 of any one of the antibodies described in Table 1a. In some embodiments, the CD79b-binding arm comprises a heavy chain comprising a CDR1, a CDR2, and a CDR3 of any one of the antibodies described in Table 1a and a light chain comprising a CDR1, a CDR2, and a CDR3 of any one of the antibodies described in Table 1a.

In some embodiments, the CD79b-binding arm comprises a heavy chain comprising a heavy chain variable domain of any one of the antibodies described in Table 1b. In some embodiments, the CD79b-binding arm comprises a light chain comprising a light chain variable domain of any one of the antibodies described in Table 1b. In some embodiments, the CD79b-binding arm comprises a heavy chain comprising a heavy chain variable domain of any one of the antibodies described in Table 1b and a light chain comprising a light chain variable domain of any one of the antibodies described in Table 1b.

In some embodiments, the CD79b-binding arm comprises a heavy chain CDR1 comprising SEQ ID NO: 1, a heavy chain CDR2 comprising SEQ ID NO: 2, and a heavy chain CDR3 comprising SEQ ID NO: 3. In some embodiments, the CD79b-binding arm comprises a heavy chain CDR1 comprising SEQ ID NO: 1, a heavy chain CDR2 comprising SEQ ID NO: 2, a heavy chain CDR3 comprising SEQ ID NO: 3, a light chain CDR1 comprising SEQ ID NO: 4, a light chain CDR2 comprising SEQ ID NO: 5, and a light chain CDR3 comprising SEQ ID NO: 6. The CD79b-binding arm may comprise human framework sequences. In some embodiments, the CD79b-binding arm comprises a heavy chain variable domain substantially the same as, or identical to, SEQ ID NO: 35. In some embodiments, the CD79b-binding arm comprises a heavy chain variable domain substantially the same as, or identical to, SEQ ID NO: 35 and a light chain variable domain substantially the same as, or identical to, SEQ ID NO: 37. In some embodiments, the CD79b-binding arm comprises a heavy chain variable domain that is encoded by a nucleotide sequence substantially the same as, or identical to, SEQ ID NO: 36. In some embodiments, the CD79b-binding arm comprises a heavy chain variable domain that is encoded by a nucleotide sequence substantially the same as, or identical to, SEQ ID NO: 36 and a light chain variable domain that is encoded by a nucleotide sequence substantially the same as, or identical to, SEQ ID NO: 38 or 213.

In some embodiments, the CD79b-binding arm comprises a heavy chain CDR1 comprising SEQ ID NO: 13, a heavy chain CDR2 comprising SEQ ID NO: 8, and a heavy chain CDR3 comprising SEQ ID NO: 9. In some embodiments, the CD79b-binding arm comprises a heavy chain CDR1 comprising SEQ ID NO: 13, a heavy chain CDR2 comprising SEQ ID NO: 8, a heavy chain CDR3 comprising SEQ ID NO: 9, a light chain CDR1 comprising SEQ ID NO: 10, a light chain CDR2 comprising SEQ ID NO: 11, and a light chain CDR3 comprising SEQ ID NO: 12. The CD79b-binding arm may comprise human framework sequences. In some embodiments, the CD79b-binding arm comprises a heavy chain variable domain substantially the same as, or identical to, SEQ ID NO: 39. In some embodiments, the CD79b-binding arm comprises a heavy chain variable domain substantially the same as, or identical to, SEQ ID NO: 39 and a light chain variable domain substantially the same as, or identical to, SEQ ID NO: 41. In some embodiments, the CD79b-binding arm comprises a heavy chain variable domain that is encoded by a nucleotide sequence substantially the same as, or identical to, SEQ ID NO: 40. In some embodiments, the CD79b-binding arm comprises a heavy chain variable domain that is encoded by a nucleotide sequence substantially the same as, or identical to, SEQ ID NO: 40 and a light chain variable domain that is encoded by a nucleotide sequence substantially the same as, or identical to, SEQ ID NO: 42.

In some embodiments, the CD79b-binding arm comprises a heavy chain CDR1 comprising SEQ ID NO: 7, a heavy chain CDR2 comprising SEQ ID NO: 8, and a heavy chain CDR3 comprising SEQ ID NO: 9. In some embodiments, the CD79b-binding arm comprises a heavy chain CDR1 comprising SEQ ID NO: 7, a heavy chain CDR2 comprising SEQ ID NO: 8, a heavy chain CDR3 comprising SEQ ID NO: 9, a light chain CDR1 comprising SEQ ID NO: 10, a light chain CDR2 comprising SEQ ID NO: 11, and a light chain CDR3 comprising SEQ ID NO: 12. The CD79b-binding arm may comprise human framework sequences. In some embodiments, the CD79b-binding arm comprises a heavy chain variable domain substantially the same as, or identical to, SEQ ID NO: 43. In some embodiments, the CD79b-binding arm comprises a heavy chain variable domain substantially the same as, or identical to, SEQ ID NO: 43 and a light chain variable domain substantially the same as, or identical to, SEQ ID NO: 41. In some embodiments, the CD79b-binding arm comprises a heavy chain variable domain that is encoded by a nucleotide sequence substantially the same as, or identical to, SEQ ID NO: 44. In some embodiments, the CD79b-binding arm comprises a heavy chain variable domain that is encoded by a nucleotide sequence substantially the same as, or identical to, SEQ ID NO: 44 and a light chain variable domain that is encoded by a nucleotide sequence substantially the same as, or identical to, SEQ ID NO: 34.

In some embodiments, the CD79b-binding arm comprises a heavy chain CDR1 comprising SEQ ID NO: 14, a heavy chain CDR2 comprising SEQ ID NO: 15, and a heavy chain CDR3 comprising SEQ ID NO: 16. In some embodiments, the CD79b-binding arm comprises a heavy chain CDR1 comprising SEQ ID NO: 14, a heavy chain CDR2 comprising SEQ ID NO: 15, a heavy chain CDR3 comprising SEQ ID NO: 16, a light chain CDR1 comprising SEQ ID NO: 17, a light chain CDR2 comprising SEQ ID NO: 5, and a light chain CDR3 comprising SEQ ID NO: 6. The CD79b-binding arm may comprise human framework sequences. In some embodiments, the CD79b-binding arm comprises a heavy chain variable domain substantially the same as, or identical to, SEQ ID NO: 45. In some embodiments, the CD79b-binding arm comprises a heavy chain variable domain substantially the same as, or identical to, SEQ ID NO: 45 and a light chain variable domain substantially the same as, or identical to, SEQ ID NO: 47. In some embodiments, the CD79b-binding arm comprises a heavy chain variable domain that is encoded by a nucleotide sequence substantially the same as, or identical to, SEQ ID NO: 46. In some embodiments, the CD79b-binding arm comprises a heavy chain variable domain that is encoded by a nucleotide sequence substantially the same as, or identical to, SEQ ID NO: 46 and a light chain variable domain that is encoded by a nucleotide sequence substantially the same as, or identical to, SEQ ID NO: 48 or 214.

In some embodiments, the CD79b-binding arm comprises a heavy chain CDR1 comprising SEQ ID NO: 18, a heavy chain CDR2 comprising SEQ ID NO: 8, and a heavy chain CDR3 comprising SEQ ID NO: 19. In some embodiments, the CD79b-binding arm comprises a heavy chain CDR1 comprising SEQ ID NO: 18, a heavy chain CDR2 comprising SEQ ID NO: 8, a heavy chain CDR3 comprising SEQ ID NO: 19, a light chain CDR1 comprising SEQ ID NO: 20, a light chain CDR2 comprising SEQ ID NO: 21, and a light chain CDR3 comprising SEQ ID NO: 12. The CD79b-binding arm may comprise human framework sequences. In some embodiments, the CD79b-binding arm comprises a heavy chain variable domain substantially the same as, or identical to, SEQ ID NO: 49. In some embodiments, the CD79b-binding arm comprises a heavy chain variable domain substantially the same as, or identical to, SEQ ID NO: 49 and a light chain variable domain substantially the same as, or identical to, SEQ ID NO: 51. In some embodiments, the CD79b-binding arm comprises a heavy chain variable domain that is encoded by a nucleotide sequence substantially the same as, or identical to, SEQ ID NO: 50. In some embodiments, the CD79b-binding arm comprises a heavy chain variable domain that is encoded by a nucleotide sequence substantially the same as, or identical to, SEQ ID NO: 50 and a light chain variable domain that is encoded by a nucleotide sequence substantially the same as, or identical to, SEQ ID NO: 52.

In some embodiments, the CD79b-binding arm comprises a heavy chain CDR1 comprising SEQ ID NO: 13, a heavy chain CDR2 comprising SEQ ID NO: 8, and a heavy chain CDR3 comprising SEQ ID NO: 9. In some embodiments, the CD79b-binding arm comprises a heavy chain CDR1 comprising SEQ ID NO: 13, a heavy chain CDR2 comprising SEQ ID NO: 8, a heavy chain CDR3 comprising SEQ ID NO: 9, a light chain CDR1 comprising SEQ ID NO: 10, a light chain CDR2 comprising SEQ ID NO: 11, and a light chain CDR3 comprising SEQ ID NO: 12. The CD79b-binding arm may comprise human framework sequences. In some embodiments, the CD79b-binding arm comprises a heavy chain variable domain substantially the same as, or identical to, SEQ ID NO: 39. In some embodiments, the CD79b-binding arm comprises a heavy chain variable domain substantially the same as, or identical to, SEQ ID NO: 39 and a light chain variable domain substantially the same as, or identical to, SEQ ID NO: 53. In some embodiments, the CD79b-binding arm comprises a heavy chain variable domain that is encoded by a nucleotide sequence substantially the same as, or identical to, SEQ ID NO: 40. In some embodiments, the CD79b-binding arm comprises a heavy chain variable domain that is encoded by a nucleotide sequence substantially the same as, or identical to, SEQ ID NO: 40 and a light chain variable domain that is encoded by a nucleotide sequence substantially the same as, or identical to, SEQ ID NO: 54.

In some embodiments, the CD79b-binding arm comprises a heavy chain CDR1 comprising SEQ ID NO: 13, a heavy chain CDR2 comprising SEQ ID NO: 8, and a heavy chain CDR3 comprising SEQ ID NO: 9. In some embodiments, the CD79b-binding arm comprises a heavy chain CDR1 comprising SEQ ID NO: 13, a heavy chain CDR2 comprising SEQ ID NO: 8, a heavy chain CDR3 comprising SEQ ID NO: 9, a light chain CDR1 comprising SEQ ID NO: 10, a light chain CDR2 comprising SEQ ID NO: 11, and a light chain CDR3 comprising SEQ ID NO: 12. The CD79b-binding arm may comprise human framework sequences. In some embodiments, the CD79b-binding arm comprises a heavy chain variable domain substantially the same as, or identical to, SEQ ID NO: 55. In some embodiments, the CD79b-binding arm comprises a heavy chain variable domain substantially the same as, or identical to, SEQ ID NO: 55 and a light chain variable domain substantially the same as, or identical to, SEQ ID NO: 57. In some embodiments, the CD79b-binding arm comprises a heavy chain variable domain that is encoded by a nucleotide sequence substantially the same as, or identical to, SEQ ID NO: 56. In some embodiments, the CD79b-binding arm comprises a heavy chain variable domain that is encoded by a nucleotide sequence substantially the same as, or identical to, SEQ ID NO: 56 and a light chain variable domain that is encoded by a nucleotide sequence substantially the same as, or identical to, SEQ ID NO: 58.

In some embodiments, the CD79b-binding arm comprises a heavy chain CDR1 comprising SEQ ID NO: 22, a heavy chain CDR2 comprising SEQ ID NO: 23, and a heavy chain CDR3 comprising SEQ ID NO: 24. In some embodiments, the CD79b-binding arm comprises a heavy chain CDR1 comprising SEQ ID NO: 22, a heavy chain CDR2 comprising SEQ ID NO: 23, a heavy chain CDR3 comprising SEQ ID NO: 24, a light chain CDR1 comprising SEQ ID NO: 25, a light chain CDR2 comprising SEQ ID NO: 5, and a light chain CDR3 comprising SEQ ID NO: 6. The CD79b-binding arm may comprise human framework sequences. In some embodiments, the CD79b-binding arm comprises a heavy chain variable domain substantially the same as, or identical to, SEQ ID NO: 59. In some embodiments, the CD79b-binding arm comprises a heavy chain variable domain substantially the same as, or identical to, SEQ ID NO: 59 and a light chain variable domain substantially the same as, or identical to, SEQ ID NO: 61. In some embodiments, the CD79b-binding arm comprises a heavy chain variable domain that is encoded by a nucleotide sequence substantially the same as, or identical to, SEQ ID NO: 60. In some embodiments, the CD79b-binding arm comprises a heavy chain variable domain that is encoded by a nucleotide sequence substantially the same as, or identical to, SEQ ID NO: 60 and a light chain variable domain that is encoded by a nucleotide sequence substantially the same as, or identical to, SEQ ID NO: 62.

In some embodiments, the CD79b-binding arm comprises a heavy chain CDR1 comprising SEQ ID NO: 22, a heavy chain CDR2 comprising SEQ ID NO: 26, and a heavy chain CDR3 comprising SEQ ID NO: 27. In some embodiments, the CD79b-binding arm comprises a heavy chain CDR1 comprising SEQ ID NO: 22, a heavy chain CDR2 comprising SEQ ID NO: 26, a heavy chain CDR3 comprising SEQ ID NO: 27, a light chain CDR1 comprising SEQ ID NO: 28, a light chain CDR2 comprising SEQ ID NO: 5, and a light chain CDR3 comprising SEQ ID NO: 29. The CD79b-binding arm may comprise human framework sequences. In some embodiments, the CD79b-binding arm comprises a heavy chain variable domain substantially the same as, or identical to, SEQ ID NO: 63. In some embodiments, the CD79b-binding arm comprises a heavy chain variable domain substantially the same as, or identical to, SEQ ID NO: 63 and a light chain variable domain substantially the same as, or identical to, SEQ ID NO: 65. In some embodiments, the CD79b-binding arm comprises a heavy chain variable domain that is encoded by a nucleotide sequence substantially the same as, or identical to, SEQ ID NO: 64. In some embodiments, the CD79b-binding arm comprises a heavy chain variable domain that is encoded by a nucleotide sequence substantially the same as, or identical to, SEQ ID NO: 64 and a light chain variable domain that is encoded by a nucleotide sequence substantially the same as, or identical to, SEQ ID NO: 66.

In some embodiments, the CD79b-binding arm comprises a heavy chain CDR1 comprising SEQ ID NO: 14, a heavy chain CDR2 comprising SEQ ID NO: 15, and a heavy chain CDR3 comprising SEQ ID NO: 16. In some embodiments, the CD79b-binding arm comprises a heavy chain CDR1 comprising SEQ ID NO: 14, a heavy chain CDR2 comprising SEQ ID NO: 15, a heavy chain CDR3 comprising SEQ ID NO: 16, a light chain CDR1 comprising SEQ ID NO: 17, a light chain CDR2 comprising SEQ ID NO: 5, and a light chain CDR3 comprising SEQ ID NO: 6. The CD79b-binding arm may comprise human framework sequences. In some embodiments, the CD79b-binding arm comprises a heavy chain variable domain substantially the same as, or identical to, SEQ ID NO: 67. In some embodiments, the CD79b-binding arm comprises a heavy chain variable domain substantially the same as, or identical to, SEQ ID NO: 67 and a light chain variable domain substantially the same as, or identical to, SEQ ID NO: 69. In some embodiments, the CD79b-binding arm comprises a heavy chain variable domain that is encoded by a nucleotide sequence substantially the same as, or identical to, SEQ ID NO: 68. In some embodiments, the CD79b-binding arm comprises a heavy chain variable domain that is encoded by a nucleotide sequence substantially the same as, or identical to, SEQ ID NO: 68 and a light chain variable domain that is encoded by a nucleotide sequence substantially the same as, or identical to, SEQ ID NO: 70.

In some embodiments, the CD79b-binding arm comprises a heavy chain CDR1 comprising SEQ ID NO: 30, a heavy chain CDR2 comprising SEQ ID NO: 31, and a heavy chain CDR3 comprising SEQ ID NO: 32. In some embodiments, the CD79b-binding arm comprises a heavy chain CDR1 comprising SEQ ID NO: 30, a heavy chain CDR2 comprising SEQ ID NO: 31, a heavy chain CDR3 comprising SEQ ID NO: 32, a light chain CDR1 comprising SEQ ID NO: 33, a light chain CDR2 comprising SEQ ID NO: 5, and a light chain CDR3 comprising SEQ ID NO: 6. The CD79b-binding arm may comprise human framework sequences. In some embodiments, the CD79b-binding arm comprises a heavy chain variable domain substantially the same as, or identical to, SEQ ID NO: 71. In some embodiments, the CD79b-binding arm comprises a heavy chain variable domain substantially the same as, or identical to, SEQ ID NO: 71 and a light chain variable domain substantially the same as, or identical to, SEQ ID NO: 73. In some embodiments, the CD79b-binding arm comprises a heavy chain variable domain that is encoded by a nucleotide sequence substantially the same as, or identical to, SEQ ID NO: 72. In some embodiments, the CD79b-binding arm comprises a heavy chain variable domain that is encoded by a nucleotide sequence substantially the same as, or identical to, SEQ ID NO: 72 and a light chain variable domain that is encoded by a nucleotide sequence substantially the same as, or identical to, SEQ ID NO: 74.

The CD79b-binding arm may be derived from any species by recombinant means. For example, the CD79b antigen-binding arm may be derived from mouse, rat, goat, horse, swine, bovine, chicken, rabbit, camelid, donkey, human, or chimeric versions thereof. For use in administration to humans, non-human derived antigen-binding fragments may be genetically or structurally altered to be less antigenic upon administration to a human patient. In some embodiments, the CD79b-binding arm comprises antigen-binding fragments which is chimeric.

In some embodiments, the CD79b-binding arm comprises humanized antigen-binding fragments. Humanized antigen-binding fragments may be derived from chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies or antigen-binding fragments are human immunoglobulins (recipient antibody) or antigen-binding fragments in which residues from a complementary-determining region (CDR) of the recipient are replaced by residues from a CDR of a non-human species (donor antibody) such as mouse, rat or rabbit having the desired specificity, affinity, and capacity. In general, the humanized antibody antigen-binding fragments will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody antigen-binding fragments may include at least a portion of an immunoglobulin constant region (Fc), typically that of a human immunoglobulin.

### CD20-Binding Arm

The multispecific antibodies (e.g., trispecific or bispecific antibodies) described herein may comprise an antigen-binding arm specific for CD20. In some embodiments, the CD20-binding arm binds human CD20. In some embodiments, CD20-binding arm binds human CD20 and cynomolgus monkey CD20, preferably the extracellular domain thereof. In some embodiments, CD20-binding arm binds human CD20 but not to cynomolgus monkey CD20. In some embodiments, the CD20-binding arm binds to the epitope on CD20 as rituximab. In some embodiments, the CD20-binding arm may bind to CD20 with an affinity of 5×10⁻⁷M or less, such as 1×10⁻⁷M or less, 5×10⁻⁸M or less, 1×10⁻⁸M or less, 5×10⁻⁹M or less, or 1×10⁻⁹M or less. In one embodiment, the CD20-binding arm binds to CD20 with an affinity of about 1×10⁻⁹M, about 2×10⁻⁹M, about 3×10⁻⁹M, about 4×10⁻⁹M, about 5×10⁻⁹M, about 6×10⁻⁹M, about 7×10⁻⁹M, about 8×10⁻⁹M, about 9×10⁻⁹M, about 1×10⁻⁸M, about 2×10⁻⁸M, about 3×10⁻⁸M, about 4×10⁻⁸M, about 5×10⁻⁸M, about 6×10⁻⁸M, about 7×10⁻⁸M, about 8×10⁻⁸M, about 9×10⁻⁸M, or about 1×10⁻⁷M.

In some embodiments, the CD20-binding arm comprises a heavy chain CDR1, CDR2, and CDR3 derived from an antibody clone as described in Table 2a. In some embodiments, the CD20-binding arm comprises a light chain CDR1, CDR2, and CDR3 derived from an antibody clone as described in Table 2a. In some embodiments, the CD20-binding arm comprises heavy chain CDR1, CDR2, and CDR3 and light chain CDR1, CDR2, and CDR3 derived from an antibody clone as described in Table 2a. In some exemplary embodiments, the CD20-binding arm comprises heavy chain CDR1, CDR2, and CDR3 and light chain CDR1, CDR2, and CDR3 of clone C20B648.

In some exemplary embodiments, the CD20-binding arm comprises a heavy chain variable domain derived from an antibody clone as described in Table 2b. In some exemplary embodiments, the CD20-binding arm comprises heavy chain variable domain and light chain variable domain derived from an antibody clone as described in Table 2b.

Table 2a and Table 2b provide a summary of CDR and VH and VL sequences of some exemplary CD20-specific antibodies described herein:

TABLE 2a
CDR sequences of exemplary mAbs generated against human CD20

| ID | HC-CDR1 | HC-CDR2 | HC-CDR3 | LC-CDR1 | LC-CDR2 | LC-CDR3 |
|---|---|---|---|---|---|---|
| C20B22 | GYTFTSYN | AIYPGNGDT | STYYGGDW | RASSSVSYI | ATSNLAS | QQWTSNPP |
| | MH (SEQ ID | S (SEQ ID | YFNV (SEQ | H (SEQ ID | (SEQ ID NO: | T (SEQ ID |
| | NO: 115) | NO: 116) | ID NO: 117) | NO: 118) | 119) | NO: 120) |
| C20B648 | GYTFSSYN | AIYPGAGDT | SNYYGSSG | RASLSVSSM | ATSNLAS | QQWIFNPPT |
| | MH (SEQ ID | S (SEQ ID | WYFDV | H (SEQ ID | (SEQ ID NO: | (SEQ ID NO: |
| | NO: 121) | NO: 122) | (SEQ ID NO: | NO: 124) | 119) | 125) |
| | | | 123) | | | |
| 5010GL | GYTFTSYN | AIYPGNGDT | VYYGSNYW | RASSSVSY | ATSNLAS | QQWIFNPPT |
| | MH (SEQ ID | S (SEQ ID | YFDV (SEQ | MH (SEQ ID | (SEQ ID NO: | (SEQ ID NO: |
| | NO: 115) | NO: 116) | ID NO: 95) | NO: 96) | 119) | 125) |
| 4A16GL | GYTFSSYN | AIYPGNGDT | SNYYGSSG | RASLSVSSM | ATSNLAS | QQWIFNPPT |
| | MH (SEQ ID | S (SEQ ID | WYFDV | H (SEQ ID | (SEQ ID NO: | (SEQ ID NO: |
| | NO: 121) | NO: 116) | (SEQ ID NO: | NO: 124) | 119) | 125) |
| | | | 123) | | | |

TABLE 2b
VH and VL sequences of exemplary mAbs generated
against human CD20

| | VH amino acid | VH DNA | VL amino | VL DNA |
|---|---|---|---|---|
| | sequence | sequence | acid | sequence |
| | SEQ ID | SEQ | sequence | SEQ |
| ID | NO | ID NO | SEQ ID NO | ID NO |
| C20B22 | 126 | 127 | 128 | 129 |
| C20B648 | 130 | 131 | 132 | 133 |
| 5O10GL | 134 | 135 | 136 | 137 |
| 4A16GL | 138 | 139 | 140 | 141 |

In some embodiments, the CD20-binding arm comprises a heavy chain variable domain comprising a CDR1, a CDR2, and a CDR3 of any one of the antibodies described in Table 2a. In some embodiments, the CD20-binding arm comprises a light chain variable domain comprising a CDR1, a CDR2, and a CDR3 of any one of the antibodies described in Table 2a. In some embodiments, the CD20-binding arm comprises a heavy chain variable domain comprising a CDR1, a CDR2, and a CDR3 of any one of the antibodies described in Table 2a and a light chain variable domain comprising a CDR1, a CDR2, and a CDR3 of any one of the antibodies described in Table 2a. In some embodiments, the CD20-binding arm competes for binding to CD20 with an antibody or antigen-binding comprising a heavy chain comprising a CDR1, a CDR2, and a CDR3 of any one of the antibodies described in Table 2a and a light chain comprising a CDR1, a CDR2, and a CDR3 of any one of the antibodies described in Table 2a.

In some embodiments, the CD20-binding arm comprises a heavy chain variable domain of any one of the antibodies described in Table 2b. In some embodiments, the CD20-binding arm comprises a light chain variable region of any one of the antibodies described in Table 2b. In some embodiments, the CD20-binding arm comprises a heavy chain variable domain of any one of the antibodies described in Table 2b and a light chain variable region of any one of the antibodies described in Table 2b. In some embodiments, the CD20-binding arm competes for binding to CD20 with an antibody or antigen-binding comprising a heavy chain variable domain of any one of the antibodies described in Table 2b and a light chain variable domain of any one of the antibodies described in Table 2b.

In some embodiments, the CD20-binding arm comprises a heavy chain comprising a CDR1, a CDR2, and a CDR3 of any one of the antibodies described in Table 2a. In some embodiments, the CD20-binding arm comprises a light chain comprising a CDR1, a CDR2, and a CDR3 of any one of the antibodies described in Table 2a. In some embodiments, the CD20-binding arm comprises a heavy chain comprising a CDR1, a CDR2, and a CDR3 of any one of the antibodies described in Table 2 and a light chain comprising a CDR1, a CDR2, and a CDR3 of any one of the antibodies described in Table 2a.

In some embodiments, the CD20-binding arm comprises a heavy chain comprising a heavy chain variable domain of any one of the antibodies described in Table 2b. In some embodiments, the CD20-binding arm comprises a light chain comprising a light chain variable domain of any one of the antibodies described in Table 2b. In some embodiments, the CD20-binding arm comprises a heavy chain comprising a heavy chain variable domain of any one of the antibodies described in Table 2b and a light chain comprising a light chain variable domain of any one of the antibodies described in Table 2b.

In some embodiments, the CD20-binding arm comprises a heavy chain CDR1 comprising SEQ ID NO: 115, a heavy chain CDR2 comprising SEQ ID NO: 116, and a heavy chain CDR3 comprising SEQ ID NO: 117. In some embodiments, the CD20-binding arm comprises a heavy chain CDR1 comprising SEQ ID NO: 115, a heavy chain CDR2 comprising SEQ ID NO: 116, a heavy chain CDR3 comprising SEQ ID NO: 117, a light chain CDR1 comprising SEQ ID NO: 118, a light chain CDR2 comprising SEQ ID NO: 119, and a light chain CDR3 comprising SEQ ID NO: 120. The CD20-binding arm may comprise human framework sequences. In some embodiments, the CD20-binding arm comprises a heavy chain variable domain substantially the same as, or identical to, SEQ ID NO: 126. In some embodiments, the CD20-binding arm comprises a heavy chain variable domain substantially the same as, or identical to, SEQ ID NO: 126 and a light chain variable domain substantially the same as, or identical to, SEQ ID NO: 128. In some embodiments, the CD20-binding arm comprises a heavy chain variable domain that is encoded by a nucleotide sequence substantially the same as, or identical to, SEQ ID NO: 127. In some embodiments, the CD20-binding arm comprises a heavy chain variable domain that is encoded by a nucleotide sequence substantially the same as, or identical to, SEQ ID NO: 127 and a light chain variable domain that is encoded by a nucleotide sequence substantially the same as, or identical to, SEQ ID NO: 129.

In some embodiments, the CD20-binding arm comprises a heavy chain CDR1 comprising SEQ ID NO: 121, a heavy chain CDR2 comprising SEQ ID NO: 122, and a heavy chain CDR3 comprising SEQ ID NO: 123. In some embodiments, the CD20-binding arm comprises a heavy chain CDR1 comprising SEQ ID NO: 121, a heavy chain CDR2 comprising SEQ ID NO: 122, a heavy chain CDR3 comprising SEQ ID NO: 123, a light chain CDR1 comprising SEQ ID NO: 124, a light chain CDR2 comprising SEQ ID NO: 119, and a light chain CDR3 comprising SEQ ID NO: 125. The CD20-binding arm may comprise human framework sequences. In some embodiments, the CD20-binding arm comprises a heavy chain variable domain substantially the same as, or identical to, SEQ ID NO: 130. In some embodiments, the CD20-binding arm comprises a heavy chain variable domain substantially the same as, or identical to, SEQ ID NO: 130 and a light chain variable domain substantially the same as, or identical to, SEQ ID NO: 132. In some embodiments, the CD20-binding arm comprises a heavy chain variable domain that is encoded by a nucleotide sequence substantially the same as, or identical to, SEQ ID NO: 131. In some embodiments, the CD20-binding arm comprises a heavy chain variable domain that is encoded by a nucleotide sequence substantially the same as, or identical to, SEQ ID NO: 131 and a light chain variable domain that is encoded by a nucleotide sequence substantially the same as, or identical to, SEQ ID NO: 133.

In some embodiments, the CD20-binding arm comprises a heavy chain CDR1 comprising SEQ ID NO: 115, a heavy chain CDR2 comprising SEQ ID NO: 116, and a heavy chain CDR3 comprising SEQ ID NO: 95. In some embodiments, the CD20-binding arm comprises a heavy chain CDR1 comprising SEQ ID NO: 115, a heavy chain CDR2 comprising SEQ ID NO: 116, a heavy chain CDR3 comprising SEQ ID NO: 95, a light chain CDR1 comprising SEQ ID NO: 96, a light chain CDR2 comprising SEQ ID NO: 119, and a light chain CDR3 comprising SEQ ID NO: 125. The CD20-binding arm may comprise human framework sequences. In some embodiments, the CD20-binding arm comprises a heavy chain variable domain substantially the same as, or identical to, SEQ ID NO: 134. In some embodiments, the CD20-binding arm comprises a heavy chain variable domain substantially the same as, or identical to, SEQ ID NO: 134 and a light chain variable domain substantially the same as, or identical to, SEQ ID NO: 136. In some embodiments, the CD20-binding arm comprises a heavy chain variable domain that is encoded by a nucleotide sequence substantially the same as, or identical to, SEQ ID NO: 135. In some embodiments, the CD20-binding arm comprises a heavy chain variable domain that is encoded by a nucleotide sequence substantially the same as, or identical to, SEQ ID NO: 135 and a light chain variable domain that is encoded by a nucleotide sequence substantially the same as, or identical to, SEQ ID NO: 137.

In some embodiments, the CD20-binding arm comprises a heavy chain CDR1 comprising SEQ ID NO: 121, a heavy chain CDR2 comprising SEQ ID NO: 116, and a heavy chain CDR3 comprising SEQ ID NO: 123. In some embodiments, the CD20-binding arm comprises a heavy chain CDR1 comprising SEQ ID NO: 121, a heavy chain CDR2 comprising SEQ ID NO: 116, a heavy chain CDR3 comprising SEQ ID NO: 123, a light chain CDR1 comprising SEQ ID NO: 124, a light chain CDR2 comprising SEQ ID NO: 119, and a light chain CDR3 comprising SEQ ID NO: 125. The CD20-binding arm may comprise human framework sequences. In some embodiments, the CD20-binding arm comprises a heavy chain variable domain substantially the same as, or identical to, SEQ ID NO: 138. In some embodiments, the CD20-binding arm comprises a heavy chain variable domain substantially the same as, or identical to, SEQ ID NO: 138 and a light chain variable domain substantially the same as, or identical to, SEQ ID NO: 140. In some embodiments, the CD20-binding arm comprises a heavy chain variable domain that is encoded by a nucleotide sequence substantially the same as, or identical to, SEQ ID NO: 139. In some embodiments, the CD20-binding arm comprises a heavy chain variable domain that is encoded by a nucleotide sequence substantially the same as, or identical to, SEQ ID NO: 139 and a light chain variable domain that is encoded by a nucleotide sequence substantially the same as, or identical to, SEQ ID NO: 141.

The CD20-binding arm may be derived from any species by recombinant means. For example, the CD20 antigen-binding arm may be derived from mouse, rat, goat, horse, swine, bovine, chicken, rabbit, camelid, donkey, human, or chimeric versions thereof. For use in administration to humans, non-human derived antigen-binding fragments may be genetically or structurally altered to be less antigenic upon administration to a human patient. In some embodiments, the CD20-binding arm comprises antigen-binding fragments which is chimeric.

In some embodiments, the CD20-binding arm comprises humanized antigen-binding fragments. Humanized antigen-binding fragments may be derived from chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies or antigen-binding fragments are human immunoglobulins (recipient antibody) or antigen-binding fragments in which residues from a complementary-determining region (CDR) of the recipient are replaced by residues from a CDR of a non-human species (donor antibody) such as mouse, rat or rabbit having the desired specificity, affinity, and capacity. In general, the humanized antibody antigen-binding fragments will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody antigen-binding fragments may include at least a portion of an immunoglobulin constant region (Fc), typically that of a human immunoglobulin.

### CD3-Binding Arm

The multispecific antibodies (e.g., trispecific or bispecific antibodies) described herein comprise an antigen-binding arm that binds CD3. In some preferred embodiments, the CD3-specific arm of the multispecific antibodies described herein is derived from a CD3-specific antibody that binds and activates human primary T cells and/or cynomolgus monkey primary T cells. In some embodiments, the CD3-binding arm binds to an epitope at the N-terminus of CD3ε. In some embodiments, the CD3-binding arm binds to residues 54-58 (GSEIL; SEQ ID NO: 257), residues 74-75 (NI), and/or residues 100-105 (PRGSKP; SEQ ID NO: 258) of human CD3ε. In some embodiments, the CD3-binding arm binds to residues 22-35 (QDGNEEMGGITQTP (SEQ ID NO: 256)) of the CD3e chain. Such CD3-binding arms may bind to CD3 with an affinity of 5×10⁻⁷M or less, such as 1×10⁻⁷M or less, 5×10⁻⁸M or less, 1×10⁻⁸M or less, 5×10⁻⁹M or less, or 1×10⁻⁹M or less. In one embodiment, the CD3-binding arm binds to the CD3 with an affinity of about 1×10⁻⁸M, about 2×10⁻⁸M, about 3×10⁻⁸M, about 4×10⁻⁸M, about 5×10⁻⁸M, about 6×10⁻⁸M, about 7×10⁻⁸M, about 8×10⁻⁸M, about 9×10⁻⁸M, or about 1×10⁻⁸M.

In some embodiments, such CD3-binding arms may have weak binding (e.g., low micromolar or weaker) to CD3. Weak CD3 binding may result in better T cell fitness, lesser T-cell exhaustion, lower risk for cytokine release syndrome (CRS), better safety, and/or may enable co-stimulatory combinations to enhance T cell persistence.

Human CD3ε is described under UniProt P07766 (CD3E_HUMAN). An anti CD3ε antibody described in the state of the art is SP34 (Yang S J, The Journal of Immunology (1986) 137; 1097-1100). SP34 reacts with both primate and human CD3. SP34 is available from Pharmingen. A further anti CD3 antibody described in the state of the art is UCHT-1 (see WO2000041474). A further anti-CD3 antibody described in the state of the art is BC-3 (Fred Hutchinson Cancer Research Institute; used in Phase I/II trials of GvHD, Anasetti et al., Transplantation 54: 844 (1992)). SP34 differs from UCHT-1 and BC-3 in that SP-34 recognizes an epitope present on solely the ε chain of CD3 (see Salmeron et al., (1991) J. Immunol. 147: 3047) whereas UCHT-1 and BC-3 recognize an epitope contributed by both the F and 7 chains. The sequence of an antibody with the same sequence as of antibody SP34 is mentioned in WO2008119565, WO2008119566, WO2008119567, WO2010037836, WO2010037837 and WO2010037838. A sequence which is 96% identical to VH of antibody SP34 is mentioned in U.S. Pat. No. 8,236,308 (WO2007042261).

In some embodiments, the CD3-binding arm contacts an epitope including the six N-terminal amino acids of CD3ε. In some embodiments, the CD3-specific binding arm of the multispecific antibody is derived from the mouse monoclonal antibody SP34, a mouse IgG3/lambda isotype. In some embodiments, the CD3-binding arm comprises the CDRs of antibody SP34. Such CD3-binding arms may bind to CD3 with an affinity of 5×10⁻⁷M or less, such as 1×10⁻⁷M or less, 5×10⁻⁸M or less, 1×10⁻⁸M or less, 5×10⁻⁹M or less, or 1×10⁻⁹M or less. The CD3-specific binding arm may be a humanized version of an arm of mouse monoclonal antibody SP34. Human framework adaptation (HFA) may be used to humanize the anti-CD3 antibody from which the CD3-specific arm is derived.

In some embodiments, the CD3-binding arm comprises a heavy chain CDR1, CDR2, and CDR3 of any one of the antibodies described in Table 3. In some embodiments, the CD3-binding arm comprises a light chain CDR1, CDR2, and CDR3 of any one of the antibodies described in Table 3. In some embodiments, the CD3-binding arm comprises a heavy chain comprising a CDR1, a CDR2, and a CDR3 of any one of the antibodies described in Table 3 and a light chain comprising a CDR1, a CDR2, and a CDR3 of any one of the antibodies described in Table 3. In some embodiments of the multispecific antibodies, the CD3-binding arm comprises a heavy chain and light chain pair selected from Table 3.

Table 3 provides a summary of examples of some CD3-specific antibodies described herein:

TABLE 3
Heavy chain and light chain CDR, VH, and VL sequences of exemplary CD3-
specific antibodies and antigen-binding fragments

| ID | Heavy Chain | Light Chain |
|---|---|---|
| CD3B2030 | CDR1: GYTFTRSTMH (SEQ ID NO: 76) | CDR1: SASSSVSYMN (SEQ ID NO: 79) |
| | CDR2: YINPSSAYTN (SEQ ID NO: 77) | CDR2: DSSKLAS (SEQ ID NO: 80) |
| | CDR3: PQVHYDYNGFPY (SEQ ID NO: 78) | CDR3: QQWSRNPPT (SEQ ID NO: 81) |
| | VH: | VL: |
| | QVQLVQSGAEVKKPGSSVKVSCKASGYTFT | EIVLTQSPATLSASPGERVTLSCSASSSVSYMNW |
| | RSTMHWVKQAPGQGLEWIGYINPSSAYTNY | YQQKPGQAPRRWIYDSSKLASGVPARFSGSGSG |
| | NQKFQGRVTLTADKSTSTAYMELSSLRSED | RDYTLTISSLEPEDFAVYYCQQWSRNPPTFGGGT |
| | TAVYYCASPQVHYDYNGFPYWGQGTLVTV | KVEIK (SEQ ID NO: 99) |
| | SS (SEQ ID NO: 97) | |
| | VH DNA sequence (SEQ ID NO: 98) | VL DNA sequence (SEQ ID NO: 100) |
| CD3B2030- | CDR1: GYTFTRSTMH (SEQ ID NO: 76) | CDR1: SASSSVSYMN (SEQ ID NO: 79) |
| N106A | CDR2: YINPSSAYTN (SEQ ID NO: 77) | CDR2: DSSKLAS (SEQ ID NO: 80) |
| | CDR3: PQVHYDYAGFPY (SEQ ID NO: 75) | CDR3: QQWSRNPPT (SEQ ID NO: 81) |
| | VH: | VL: |
| | QVQLVQSGAEVKKPGSSVKVSCKASGYTFT | EIVLTQSPATLSASPGERVTLSCSASSSVSYMNW |
| | RSTMHWVKQAPGQGLEWIGYINPSSAYTNY | YQQKPGQAPRRWIYDSSKLASGVPARFSGSGSG |
| | NQKFQGRVTLTADKSTSTAYMELSSLRSED | RDYTLTISSLEPEDFAVYYCQQWSRNPPTFGGGT |
| | TAVYYCASPQVHYDYAGFPYWGQGTLVTV | KVEIK (SEQ ID NO: 99) |
| | SS (SEQ ID NO: 101) | |
| | VH DNA sequence (SEQ ID NO: 102) | VL DNA sequence (SEQ ID NO: 100) |
| CD3B2089 | CDR1: GYTFTRSTMH (SEQ ID NO: 76) | CDR1: SASSSVSYMN (SEQ ID NO: 79) |
| | CDR2: YINPSSAYTN (SEQ ID NO: 77) | CDR2: DSSKLAS (SEQ ID NO: 80) |
| | CDR3: PQVHYDYNGFPY (SEQ ID NO: 78) | CDR3: QQWSRNPPT (SEQ ID NO: 81) |
| | VH: | VL: |
| | QVQLVQSGAEVKKPGSSVKVSCKASGYTFT | EIVLTQSPATLSASPGERVTLSCSASSSVSYMNW |
| | RSTMHWVRQAPGQGLEWMGYINPSSAYTN | YQQKPGQAPRRWIYDSSKLASGVPARFSGSGSG |
| | YAQKFQGRVTLTADKSTSTAYMELSSLRSE | RDYTLTISSLEPEDFAVYYCQQWSRNPPTFGGGT |
| | DTAVYYCASPQVHYDYNGFPYWGQGTLVT | KVEIK (SEQ ID NO: 99) |
| | VSS (SEQ ID NO: 103) | |
| | VH DNA sequence (SEQ ID NO: 104) | VL DNA sequence (SEQ ID NO: 100) |
| CD3B2089- | CDR1: GYTFTRSTMH (SEQ ID NO: 76) | CDR1: SASSSVSYMN (SEQ ID NO: 79) |
| N106G | CDR2: YINPSSAYTN (SEQ ID NO: 77) | CDR2: DSSKLAS (SEQ ID NO: 80) |
| | CDR3: PQVHYDYGGFPY (SEQ ID NO: 82) | CDR3: QQWSRNPPT (SEQ ID NO: 81) |
| | VH: | VL: |
| | QVQLVQSGAEVKKPGSSVKVSCKASGYTFT | EIVLTQSPATLSASPGERVTLSCSASSSVSYMNW |
| | RSTMHWVRQAPGQGLEWMGYINPSSAYTN | YQQKPGQAPRRWIYDSSKLASGVPARFSGSGSG |
| | YAQKFQGRVTLTADKSTSTAYMELSSLRSE | RDYTLTISSLEPEDFAVYYCQQWSRNPPTFGGGT |
| | DTAVYYCASPQVHYDYGGFPYWGQGTLVT | KVEIK (SEQ ID NO: 99) |
| | VSS (SEQ ID NO: 105) | |
| | VH DNA sequence (SEQ ID NO: 106) | VL DNA sequence (SEQ ID NO: 100) |
| CD3W245 | CDR 1: GFTFSRYNMN (SEQ ID NO: 83) | CDR 1: RARQSIGTAIH (SEQ ID NO: 86) |
| | CDR 2: SISTSSNYIY (SEQ ID NO: 84) | CDR 2: YASESIS (SEQ ID NO: 87) |
| | CDR 3: GWGPFDY (SEQ ID NO: 85) | CDR 3: QQSGSWPYT (SEQ ID NO: 88) |
| | VH: | VL: |
| | EVQLVESGGGLVKPGGSLRLSCAASGFTFSR | DIQMTQSPSSLSASVGDRVTITCRARQSIGTAIHW |
| | YNMNWVRQAPGKGLEWVSSISTSSNYIYYA | YQQKPGKAPKLLIKYASESISGVPSRFSGSGSGTD |
| | DSVKGRFTFSRDNAKNSLDLQMSGLRAEDT | FTLTISSLQPEDFATYYCQQSGSWPYTFGQGTKL |
| | AIYYCTRGWGPFDYWGQGTLVTVSS (SEQ | EIK (SEQ ID NO: 109) |
| | ID NO: 107) | |
| | VH DNA sequence (SEQ ID NO: 108) | VL DNA sequence (SEQ ID NO: 110) |
| CD3B376 | CDR 1: GDSVENNNAAWS (SEQ ID NO: 193) | CDR 1: TGTSSNIGTYKFVS (SEQ ID NO: 198) |
| | CDR 2: RTYYRSKWLYD (SEQ ID NO: 194) | CDR 2: EVSKRPS (SEQ ID NO: 199) |
| | CDR 3: GYSSSFDY (SEQ ID NO: 195) | CDR 3: VSYAGSGTLL (SEQ ID NO: 200) |
| | VH: | VL: |
| | QVQLQQSGPRLVRPSQTLSLTCAISGDSVFN | QSALTQPASVSGSPGQSITISCTGTSSNIGTYKFVS |
| | NNAAWSWIRQSPSRGLEWLGRTYYRSKWL | WYQQHPDKAPKVLLYEVSKRPSGVSSRFSGSKS |
| | YDYAVSVKSRITVNPDTSRNQFTLQLNSVTP | GNTASLTISGLQAEDQADYHCVSYAGSGTLLFG |
| | EDTALYYCARGYSSSFDYWGQGTLVTVSS | GGTKLTVL (SEQ ID NO: 201) |
| | (SEQ ID NO: 196) | |
| | Heavy chain: | Light chain: |
| | QVQLQQSGPRLVRPSQTLSLTCAISGDSVFN | QSALTQPASVSGSPGQSITISCTGTSSNIGTYKFVS |
| | NNAAWSWIRQSPSRGLEWLGRTYYRSKWL | WYQQHPDKAPKVLLYEVSKRPSGVSSRFSGSKS |
| | YDYAVSVKSRITVNPDTSRNQFTLQLNSVTP | GNTASLTISGLQAEDQADYHCVSYAGSGTLLFG |
| | EDTALYYCARGYSSSFDYWGQGTLVTVSSA | GGTKLTVLGQPKAAPSVTLFPPSSEELQANKATL |
| | STKGPSVFPLAPSSKSTSGGTAALGCLVKDY | VCLISDFYPGAVTVAWKADSSPVKAGVETTTPS |
| | FPEPVTVSWNSGALTSGVHTFPAVLQSSGLY | KQSNNKYAASSYLSLTPEQWKSHRSYSCQVTHE |
| | SLSSVVTVPSSSLGTQTYICNVNHKPSNTKV | GSTVEKTVAPTECS (SEQ ID NO: 202) |
| | DKKVEPKSCDKTHTCPPCPAPEAAGGPSVFL | |
| | FPPKPKDTLMISRTPEVTCVVVSVSHEDPEV | |
| | KFNWYVDGVEVHNAKTKPREEQYNSTYRV | |
| | VSVLTVLHQDWLNGKEYKCKVSNKALPAPI | |
| | EKTISKAKGQPREPQVYTLPPSREEMTKNQV | |
| | SLSCAVKGFYPSDIAVEWESNGQPENNYKT | |
| | TPPVLDSDGSFFLVSKLTVDKSRWQQGNVF | |
| | SCSVMHEALHNRFTQKSLSLSPGK (SEQ ID | |
| | NO: 197) | |
| CD3B219 | CDR 1: TYAMN (SEQ ID NO: 203) | CDR 1: RSSTGAVTTSNYAN (SEQ ID NO: 208) |
| | CDR 2: RIRSKYNNYATYYAASVKG (SEQ ID | CDR 2: GTNKRAP (SEQ ID NO: 209) |
| | NO: 204) | |
| | CDR 3: HGNFGNSYVSWFAY (SEQ ID NO: | CDR 3: ALWYSNLWV (SEQ ID NO: 210) |
| | 205) | |
| | VH: | VL: |
| | EVQLVESGGGLVQPGGSLRLSCAASGFTFNT | QTVVTQEPSLTVSPGGTVTLTCRSSTGAVTTSNY |
| | YAMNWVRQAPGKGLEWVARIRSKYNNYA | ANWVQQKPGQAPRGLIGGTNKRAPGTPARFSGS |
| | TYYAASVKGRFTISRDDSKNSLYLQMNSLK | LLGGKAALTLSGVQPEDEAEYYCALWYSNLWV |
| | TEDTAVYYCARHGNFGNSYVSWFAYWGQG | FGGGTKLTVL (SEQ ID NO: 211) |
| | TLVTVSS (SEQ ID NO: 206) | |
| | Heavy chain: | Light chain: |
| | EVQLVESGGGLVQPGGSLRLSCAASGFTFNT | QTVVTQEPSLTVSPGGTVTLTCRSSTGAVTTSNY |
| | YAMNWVRQAPGKGLEWVARIRSKYNNYA | ANWVQQKPGQAPRGLIGGTNKRAPGTPARFSGS |
| | TYYAASVKGRFTISRDDSKNSLYLQMNSLK | LLGGKAALTLSGVQPEDEAEYYCALWYSNLWV |
| | TEDTAVYYCARHGNFGNSYVSWFAYWGQG | FGGGTKLTVLGQPKAAPSVTLFPPSSEELQANKA |
| | TLVTVSSASTKGPSVFPLAPCSRSTSESTAAL | TLVCLISDFYPGAVTVAWKADSSPVKAGVETTTP |
| | GCLVKDYFPEPVTVSWNSGALTSGVHTFPA | SKQSNNKYAASSYLSLTPEQWKSHRSYSCQVTH |
| | VLQSSGLYSLSSVVTVPSSSLGTKTYTCNVD | EGSTVEKTVAPTECS (SEQ ID NO: 212) |
| | HKPSNTKVDKRVESKYGPPCPPCPAPEAAG | |
| | GPSVFLFPPKPKDTLMISRTPEVTCVVVDVS | |
| | QEDPEVQFNWYVDGVEVHNAKTKPREEQF | |
| | NSTYRVVSVLTVLHQDWLNGKEYKCKVSN | |
| | KGLPSSIEKTISKAKGQPREPQVYTLPPSQEE | |
| | MTKNQVSLTCLVKGFYPSDIAVEWESNGQP | |
| | ENNYKTTPPVLDSDGSFLLYSKLTVDKSRW | |
| | QEGNVFSCSVMHEALHNHYTQKSLSLSLGK | |
| | (SEQ ID NO: 207) | |

Characteristics of some CD3-specific antibodies or antigen-binding fragments may be found in e.g., U.S. Pat. Nos. 10,562,968 and 10,072,088, United States Published Application US2019/0382481, the content of each of which is herein incorporated by reference in its entirety.

In some embodiments, the CD3-binding arm comprises a heavy chain variable domain comprising a CDR1, a CDR2, and a CDR3 of any one of the antibodies described in Table 3. In some embodiments, the CD3-binding arm comprises a light chain variable domain comprising a CDR1, a CDR2, and a CDR3 of any one of the antibodies described in Table 3. In some embodiments, the CD3-binding arm comprises a heavy chain variable domain comprising a CDR1, a CDR2, and a CDR3 of any one of the antibodies described in Table 3 and a light chain variable domain comprising a CDR1, a CDR2, and a CDR3 of any one of the antibodies described in Table 3. In some embodiments, the CD3-binding arm competes for binding to CD3 with an antibody or antigen-binding comprising a heavy chain comprising a CDR1, a CDR2, and a CDR3 of any one of the antibodies described in Table 3 and a light chain comprising a CDR1, a CDR2, and a CDR3 of any one of the antibodies described in Table 3.

In some embodiments, the CD3-binding arm comprises a heavy chain comprising a CDR1, a CDR2, and a CDR3 of any one of the antibodies described in Table 3. In some embodiments, the CD3-binding arm comprises a light chain comprising a CDR1, a CDR2, and a CDR3 of any one of the antibodies described in Table 3. In some embodiments, the CD3-binding arm comprises a heavy chain comprising a CDR1, a CDR2, and a CDR3 of any one of the antibodies described in Table 3 and a light chain comprising a CDR1, a CDR2, and a CDR3 of any one of the antibodies described in Table 3.

In some embodiments, the CD3-binding arm comprises a heavy chain CDR1 comprising SEQ ID NO: 76, a heavy chain CDR2 comprising SEQ ID NO: 77, and a heavy chain CDR3 comprising SEQ ID NO: 78. In some embodiments, the CD3-binding arm comprises a heavy chain CDR1 comprising SEQ ID NO: 76, a heavy chain CDR2 comprising SEQ ID NO: 77, a heavy chain CDR3 comprising SEQ ID NO: 78, a light chain CDR1 comprising SEQ ID NO: 79, a light chain CDR2 comprising SEQ ID NO: 80, and a light chain CDR3 comprising SEQ ID NO: 81. The CD3-binding arm may comprise human framework sequences. In some embodiments, the CD3-binding arm comprises a heavy chain variable domain substantially the same as, or identical to, SEQ ID NO: 97. In some embodiments, the CD3-binding arm comprises a heavy chain variable domain substantially the same as, or identical to, SEQ ID NO: 97 and a light chain variable domain substantially the same as, or identical to, SEQ ID NO: 99. In some embodiments, the CD3-binding arm comprises a heavy chain variable domain that is encoded by a nucleotide sequence substantially the same as, or identical to, SEQ ID NO: 98. In some embodiments, the CD3-binding arm comprises a heavy chain variable domain that is encoded by a nucleotide sequence substantially the same as, or identical to, SEQ ID NO: 98 and a light chain variable domain that is encoded by a nucleotide sequence substantially the same as, or identical to, SEQ ID NO: 100.

In some embodiments, the CD3-binding arm comprises a heavy chain CDR1 comprising SEQ ID NO: 76, a heavy chain CDR2 comprising SEQ ID NO: 77, and a heavy chain CDR3 comprising SEQ ID NO: 75. In some embodiments, the CD3-binding arm comprises a heavy chain CDR1 comprising SEQ ID NO: 76, a heavy chain CDR2 comprising SEQ ID NO: 77, a heavy chain CDR3 comprising SEQ ID NO: 75, a light chain CDR1 comprising SEQ ID NO: 79, a light chain CDR2 comprising SEQ ID NO: 80, and a light chain CDR3 comprising SEQ ID NO: 81. The CD3-binding arm may comprise human framework sequences. In some embodiments, the CD3-binding arm comprises a heavy chain variable domain substantially the same as, or identical to, SEQ ID NO: 101. In some embodiments, the CD3-binding arm comprises a heavy chain variable domain substantially the same as, or identical to, SEQ ID NO: 101 and a light chain variable domain substantially the same as, or identical to, SEQ ID NO: 99. In some embodiments, the CD3-binding arm comprises a heavy chain variable domain that is encoded by a nucleotide sequence substantially the same as, or identical to, SEQ ID NO: 102. In some embodiments, the CD3-binding arm comprises a heavy chain variable domain that is encoded by a nucleotide sequence substantially the same as, or identical to, SEQ ID NO: 102 and a light chain variable domain that is encoded by a nucleotide sequence substantially the same as, or identical to, SEQ ID NO: 100.

In some embodiments, the CD3-binding arm comprises a heavy chain CDR1 comprising SEQ ID NO: 76, a heavy chain CDR2 comprising SEQ ID NO: 77, and a heavy chain CDR3 comprising SEQ ID NO: 78. In some embodiments, the CD3-binding arm comprises a heavy chain CDR1 comprising SEQ ID NO: 76, a heavy chain CDR2 comprising SEQ ID NO: 77, a heavy chain CDR3 comprising SEQ ID NO: 78, a light chain CDR1 comprising SEQ ID NO: 79, a light chain CDR2 comprising SEQ ID NO: 80, and a light chain CDR3 comprising SEQ ID NO: 81. The CD3-binding arm may comprise human framework sequences. In some embodiments, the CD3-binding arm comprises a heavy chain variable domain substantially the same as, or identical to, SEQ ID NO: 103. In some embodiments, the CD3-binding arm comprises a heavy chain variable domain substantially the same as, or identical to, SEQ ID NO: 103 and a light chain variable domain substantially the same as, or identical to, SEQ ID NO: 99. In some embodiments, the CD3-binding arm comprises a heavy chain variable domain that is encoded by a nucleotide sequence substantially the same as, or identical to, SEQ ID NO: 104. In some embodiments, the CD3-binding arm comprises a heavy chain variable domain that is encoded by a nucleotide sequence substantially the same as, or identical to, SEQ ID NO: 104 and a light chain variable domain that is encoded by a nucleotide sequence substantially the same as, or identical to, SEQ ID NO: 100.

In some embodiments, the CD3-binding arm comprises a heavy chain CDR1 comprising SEQ ID NO: 76, a heavy chain CDR2 comprising SEQ ID NO: 77, and a heavy chain CDR3 comprising SEQ ID NO: 82. In some embodiments, the CD3-binding arm comprises a heavy chain CDR1 comprising SEQ ID NO: 76, a heavy chain CDR2 comprising SEQ ID NO: 77, a heavy chain CDR3 comprising SEQ ID NO: 82, a light chain CDR1 comprising SEQ ID NO: 79, a light chain CDR2 comprising SEQ ID NO: 80, and a light chain CDR3 comprising SEQ ID NO: 81. The CD3-binding arm may comprise human framework sequences. In some embodiments, the CD3-binding arm comprises a heavy chain variable domain substantially the same as, or identical to, SEQ ID NO: 105. In some embodiments, the CD3-binding arm comprises a heavy chain variable domain substantially the same as, or identical to, SEQ ID NO: 105 and a light chain variable domain substantially the same as, or identical to, SEQ ID NO: 99. In some embodiments, the CD3-binding arm comprises a heavy chain variable domain that is encoded by a nucleotide sequence substantially the same as, or identical to, SEQ ID NO: 106. In some embodiments, the CD3-binding arm comprises a heavy chain variable domain that is encoded by a nucleotide sequence substantially the same as, or identical to, SEQ ID NO: 106 and a light chain variable domain that is encoded by a nucleotide sequence substantially the same as, or identical to, SEQ ID NO: 100.

In some embodiments, the CD3-binding arm comprises a heavy chain CDR1 comprising SEQ ID NO: 83, a heavy chain CDR2 comprising SEQ ID NO: 84, and a heavy chain CDR3 comprising SEQ ID NO: 85. In some embodiments, the CD3-binding arm comprises a heavy chain CDR1 comprising SEQ ID NO: 83, a heavy chain CDR2 comprising SEQ ID NO: 84, a heavy chain CDR3 comprising SEQ ID NO: 85, a light chain CDR1 comprising SEQ ID NO: 86, a light chain CDR2 comprising SEQ ID NO: 87, and a light chain CDR3 comprising SEQ ID NO: 88. The CD3-binding arm may comprise human framework sequences. In some embodiments, the CD3-binding arm comprises a heavy chain variable domain substantially the same as, or identical to, SEQ ID NO: 107. In some embodiments, the CD3-binding arm comprises a heavy chain variable domain substantially the same as, or identical to, SEQ ID NO: 107 and a light chain variable domain substantially the same as, or identical to, SEQ ID NO: 109. In some embodiments, the CD3-binding arm comprises a heavy chain variable domain that is encoded by a nucleotide sequence substantially the same as, or identical to, SEQ ID NO: 108. In some embodiments, the CD3-binding arm comprises a heavy chain variable domain that is encoded by a nucleotide sequence substantially the same as, or identical to, SEQ ID NO: 108 and a light chain variable domain that is encoded by a nucleotide sequence substantially the same as, or identical to, SEQ ID NO: 110.

In some embodiments, the CD3-binding arm comprises a heavy chain CDR1 comprising SEQ ID NO: 193, a heavy chain CDR2 comprising SEQ ID NO: 194, and a heavy chain CDR3 comprising SEQ ID NO: 195. In some embodiments, the CD3-binding arm comprises a heavy chain CDR1 comprising SEQ ID NO: 193, a heavy chain CDR2 comprising SEQ ID NO: 194, a heavy chain CDR3 comprising SEQ ID NO: 195, a light chain CDR1 comprising SEQ ID NO: 198, a light chain CDR2 comprising SEQ ID NO: 199, and a light chain CDR3 comprising SEQ ID NO: 200. The CD3-binding arm may comprise human framework sequences. In some embodiments, the CD3-binding arm comprises a heavy chain variable domain substantially the same as, or identical to, SEQ ID NO: 196. In some embodiments, the CD3-binding arm comprises a heavy chain variable domain substantially the same as, or identical to, SEQ ID NO: 196 and a light chain variable domain substantially the same as, or identical to, SEQ ID NO: 201.

In some embodiments, the CD3-binding arm comprises a heavy chain CDR1 comprising SEQ ID NO: 203, a heavy chain CDR2 comprising SEQ ID NO: 204, and a heavy chain CDR3 comprising SEQ ID NO: 205. In some embodiments, the CD3-binding arm comprises a heavy chain CDR1 comprising SEQ ID NO: 203, a heavy chain CDR2 comprising SEQ ID NO: 204, a heavy chain CDR3 comprising SEQ ID NO: 205, a light chain CDR1 comprising SEQ ID NO: 208, a light chain CDR2 comprising SEQ ID NO: 209, and a light chain CDR3 comprising SEQ ID NO: 210. The CD3-binding arm may comprise human framework sequences. In some embodiments, the CD3-binding arm comprises a heavy chain variable domain substantially the same as, or identical to, SEQ ID NO: 206. In some embodiments, the CD3-binding arm comprises a heavy chain variable domain substantially the same as, or identical to, SEQ ID NO: 206 and a light chain variable domain substantially the same as, or identical to, SEQ ID NO: 211.

In some embodiments, the CDRs of the heavy chain and/or the light chain are derived from known anti-CD3 antibodies, such as, for example, muromonab-CD3 (OKT3), otelixizumab (TRX4), teplizumab (MGA031), visilizumab (Nuvion), TR-66 or X35-3, VIT3, BMA030 (BW264/56), CLB-T3/3, CRIS7, YTH12.5, Fl 11-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In some embodiments, the CD3-binding arm is IgG, or a derivative thereof. In some embodiments, the CD3-binding arm is IgG1, IgG2, IgG3, or IgG4. In some embodiments where in the CD3-binding arm has an IgG4 isotype, it contains S228P, L234A, L235A, F405L, and R409K substitution(s) in its Fc region. In some embodiments, the antibodies or antigen-binding fragments bind CD3ε on primary human T cells. In some embodiments, the antibodies or antigen-binding fragments bind CD3ε on primary cynomolgus T cells. In some embodiments, the antibodies or antigen-binding fragments bind CD3ε on primary human and cynomolgus T cells. In some embodiments, the antibodies or antigen-binding fragments activate primary human CD3+ T cells. In some embodiments, the antibodies or antigen-binding fragments activate primary cynomolgus CD4+ T cells.

In some embodiments, the multispecific antibodies described herein may adopt any format which has been described in the art for multispecific antibodies. In some embodiments, the multispecific antibodies described herein is constructed based on a bispecific antibody format. This can be achieved by adding a third antigen-binding arm to a bispecific antibody. Different formats of bispecific antibodies have been described and were recently reviewed by Chames and Baty (2009) Curr Opin Drug Disc Dev 12: 276. In some embodiments, the multispecific antibody comprises a bispecific antibody which is a diabody, a cross-body, or a bispecific antibody obtained via a controlled Fab arm exchange as those described in the present disclosure.

In some embodiments, the multispecific antibodies include IgG-like molecules with complementary CH3 domains to force heterodimerization; recombinant IgG-like dual targeting molecules, wherein the two sides of the molecule each contain the Fab fragment or part of the Fab fragment of at least two different antibodies; IgG fusion molecules, wherein full length IgG antibodies are fused to an extra Fab fragment or parts of Fab fragment; Fc fusion molecules, wherein single chain Fv molecules or stabilized diabodies are fused to heavy-chain constant-domains, Fc-regions or parts thereof; Fab fusion molecules, wherein different Fab-fragments are fused together; ScFv- and diabody-based and heavy chain antibodies (e.g., domain antibodies, nanobodies) wherein different single chain Fv molecules or different diabodies or different heavy-chain antibodies (e.g. domain antibodies, nanobodies) are fused to each other or to another protein or carrier molecule.

In some embodiments, IgG-like molecules with complementary CH3 domains molecules include the Triomab/Quadroma (Trion Pharma/Fresenius Biotech), the Knobs-into-Holes (Genentech), CrossMAbs (Roche) and the electrostatically-matched (Amgen), the LUZ-Y (Genentech), the Strand Exchange Engineered Domain body (SEEDbody) (EMD Serono), the Biclonic (Merus), the DuoBody (Genmab A/S), and other asymmetric mutations (e.g., Zymeworks).

In some embodiments, recombinant IgG-like dual targeting molecules include Dual Targeting (DT)-Ig (GSK/Domantis), Two-in-one Antibody (Genentech), Cross-linked Mabs (Karmanos Cancer Center), mAb2 (F-Star) and CovX-body (CovX/Pfizer).

In some embodiments, IgG fusion molecules include Dual Variable Domain (DVD)-Ig (Abbott), IgG-like Bispecific (InnClone/Eli Lilly), Ts2Ab (Medlmmune/AZ) and BsAb (Zymogenetics), HERCULES (Biogen Idec) and TvAb (Roche).

In some embodiments, Fc fusion molecules include to ScFv/Fc Fusions (Academic Institution), SCORPION (Emergent BioSolutions/Trubion, Zymogenetics/BMS), Dual Affinity Retargeting Technology (Fc-DART) (MacroGenics) and Dual(ScFv).sub.2-Fab (National Research Center for Antibody Medicine—China).

In some embodiments, Fab fusion bispecific antibodies include F(ab)2 (Medarex/AMGEN), Dual-Action or Bis-Fab (Genentech), Dock-and-Lock (DNL) (ImmunoMedics), Bivalent Bispecific (Biotecnol) and Fab-Fv (UCB-Celltech). ScFv-, diabody-based and domain antibodies include but are not limited to Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) and COMBODY (Epigen Biotech), dual targeting nanobodies (Ablynx), dual targeting heavy chain only domain antibodies.

Full length multispecific antibodies of the present disclosure may be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent mono specific antibody molecule and simultaneously CH3 domains of the parent antibodies release and reform by dissociation-association. The CH3 domains of the Fab arms may be engineered to favor heterodimerization over homodimerization. The resulting product is a bispecific antibody having two Fab arms or half molecules which each bind a distinct epitope, e.g., an epitope on CD79b (or CD20) and an epitope on CD3. A third antigen-binding arm can be then introduced to the bispecific antibody, for example, to the C-terminus of the first heavy chain or second heavy chain, which can bind to a third epitope, e.g., CD20 (or CD79b).

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Inti. Publ. No. WO 2006/028936) may be used to generate full length multispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V (EU numbering).

In some embodiments of the multispecific antibody or multispecific binding fragment described herein, one of the Fc domains comprise mutations T366S, L368A and Y407V and the other Fc domain comprises mutation T366W. In some embodiments, the Fc domain of the first heavy chain portion (HC1) of the first antigen binding arm (e.g., CD79b binding arm) comprises mutations T366S, L368A and Y407V, and the Fc domain of the second heavy chain portion (HC2) of the second antigen binding arm and/or third antigen-binding arm (e.g., the CD3/CD20 binding arm of the trispecific antibody, or CD3 binding arm of the bispecific antibody) comprises mutation T366W. In some embodiments, the Fc domain of the HC2 of the second antigen-binding arm and/or third antigen-binding arm (e.g., the CD3/CD20 binding arm of the trispecific antibody, or CD3 binding arm in the bispecific antibody) comprises mutations T366S, L368A and Y407V, and the Fc domain of the HC1 of the first antigen-binding arm (e.g., CD79b binding arm) comprises mutation T366W.

Other strategies such as promoting heavy chain heterodimerization using electrostatic interactions by substituting positively charged residues at one CH3 surface and negatively charged residues at a second CH3 surface may be used, as described in US Pat. Publ. No. US2010/0015133; US Pat. Publ. No. US2009/0182127; US Pat. Publ. No. US2010/028637 or US Pat. Publ. No. US2011/0123532. In other strategies, heterodimerization may be promoted by the following substitutions (expressed as modified position in the first CH3 domain of the first heavy chain/modified position in the second CH3 domain of the second heavy chain): L351Y_F405AY407V/T394W, T3661_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. US2012/0149876 or U.S. Pat. Publ. No. US2013/0195849 (Zymeworks).

In addition to methods described above, multispecific antibodies of the invention may be generated in vitro in a cell-free environment by introducing asymmetrical mutations in the CH3 regions of two mono specific homodimeric antibodies and forming the multispecific heterodimeric antibody from two parent monospecific homodimeric antibodies in reducing conditions to allow disulfide bond isomerization according to methods described in Inti. Pat. Publ. No. WO2011/131746. In the methods, the first monospecific bivalent antibody (e.g., anti-CD79b antibody) and the second monospecific bivalent antibody (e.g., anti-CD3 antibody) are engineered to have certain substitutions at the CH3 domain that promotes heterodimer stability; the antibodies are incubated together under reducing conditions sufficient to allow the cysteines in the hinge region to undergo disulfide bond isomerization; thereby generating the multispecific antibody by Fab arm exchange. The incubation conditions may optimally be restored to non-reducing conditions. Exemplary reducing agents that may be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 min at a temperature of at least 20° C. in the presence of at least 25 mM 2-MEA or in the presence of at least 0.5 mM dithiothreitol at a pH from 5-8, for example at pH of 7.0 or at pH of 7.4 may be used.

In some embodiments, the multispecific antibodies or antigen-binding fragments are IgG, or derivatives thereof. The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcγRs) on the surface of immune effector cells such as natural killers and macrophages, leading to the phagocytosis or lysis of the targeted cells. In CDC, the antibodies kill the targeted cells by triggering the complement cascade at the cell surface. The antibodies described herein include antibodies with the described features of the variable domains in combination with any of the IgG isotypes, including modified versions in which the Fc sequence has been modified to effect different effector functions.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcγRs or the complement factors. The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Mutations have been introduced in IgG1, IgG2 and IgG4 to reduce or silence Fc functionalities. The antibodies described herein may include these modifications.

In one embodiment, the antibody comprises an Fc region with one or more of the following properties: (a) reduced effector function when compared to the parent Fc; (b) reduced affinity to FcγRI, FcγRIIa, FcγRIIb, FcγRIIIb and/or FcγRIIIa, (c) reduced affinity to FcγRI (d) reduced affinity to FcγRIIa (e) reduced affinity to FcγRIIb, (f) reduced affinity to FcγRIIIb or (g) reduced affinity to FcγRIIIa.

In some embodiments, the antibodies or antigen-binding fragments are IgG, or derivatives thereof, e.g., IgG1, IgG2, IgG3, and IgG4 isotypes. In some embodiments wherein the antibody has an IgG1 isotype, the antibody contains L234A, L235A, D265S and/or K409R substitution(s) in its Fc region. In some embodiments wherein the antibody has an IgG4 isotype, the antibody contains S228P, L234A, and L235A substitutions in its Fc region. The antibodies described herein may include these modifications.

In some embodiments, the Fc domains of one or more of the heavy chain portions (such as HC1 and/or HC2) described herein each comprise one or more mutations selected from L234A, L235A, and D265S. In some embodiments, the Fc domains of the heavy chain portions (such as HC1 and HC2) each comprise mutations L234A, L235A, and D265S.

In some embodiments, the Fc domain of one of the heavy chain portions of a multispecific antibody described herein further comprises one or more mutations which reduce Fc binding to protein A. In some embodiments, the Fc domain of one of the heavy chain portions comprises mutations H435R and/or Y436F. In some embodiments, the Fc domain of the HC2 of the second antigen-binding arm and/or third antigen-binding arm (e.g., CD3/CD20 binding arms in a trispecific antibody, or CD3 binding arm in a bispecific antibody) comprises mutations H435R and/or Y436F.

In various embodiments of a trispecific antibody described herein, the third antigen-binding arm is operatively linked to the first antigen-binding arm or second antigen-binding arm via a linker. In some embodiments, the linker is a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the third antigen-binding arm and the first antigen-binding arm or the second antigen-binding arm in such a way that they form the correct conformation relative to one another so that they retain the desired activity, such as binding to a third antigen (e.g., CD3 or CD20).

In some embodiments of a trispecific antibody described herein, the HC1 comprises, from the N- to C-terminus, the VH1 associated with the first antigen-binding arm, a CH1 domain, the Fc domain, a linker, and the third antigen-binding arm.

In some embodiments of a trispecific antibody described herein, the HC2 comprises, from the N- to C-terminus, the second antigen-binding domain, the Fc domain, a linker, and the third antigen-binding arm.

In various embodiments, the scFvs used in multispecific antibodies described herein comprises, from the N- to C-terminus, a VH, a linker, and a VL (VH-L-VL), or the VL, the linker, and the VH (VL-L-VH). In some embodiments, the scFv comprises, from the N- to C-terminus, the VL, the linker, and the VH (VL-L-VH). In some embodiments, the scFv comprises, from the N- to C-terminus, the VH, the linker, and the VH (VL-L-VH).

Linkers used in the present disclosure may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. In some embodiments, the linker is 6 amino acids long. In some embodiments, the linker is 7 amino acids long. In some embodiments, the linker is 8 amino acids long. In some embodiments, the linker is 9 amino acids long. In some embodiments, the linker is 10 amino acids long. In some embodiments, the linker is 11 amino acids long. In some embodiments, the linker is 12 amino acids long. In some embodiments, the linker is 13 amino acids long. In some embodiments, the linker is 14 amino acids long. In some embodiments, the linker is 15 amino acids long. In some embodiments, the linker is 16 amino acids long. In some embodiments, the linker is 17 amino acids long. In some embodiments, the linker is 18 amino acids long. In some embodiments, the linker is 19 amino acids long. In some embodiments, the linker is 20 amino acids long. In some embodiments, the linker is 21 amino acids long. In some embodiments, the linker is 22 amino acids long. In some embodiments, the linker is 23 amino acids long. In some embodiments, the linker is 24 amino acids long. In some embodiments, the linker is 25 amino acids long. In some embodiments, the linker is 26 amino acids long. In some embodiments, the linker is 27 amino acids long. In some embodiments, the linker is 28 amino acids long. In some embodiments, the linker is 29 amino acids long. In some embodiments, the linker is 30 amino acids long. In some embodiments, the linker is 31 amino acids long. In some embodiments, the linker is 32 amino acids long. In some embodiments, the linker is 33 amino acids long. In some embodiments, the linker is 34 amino acids long. In some embodiments, the linker is 35 amino acids long. In some embodiments, the linker is 36 amino acids long. In some embodiments, the linker is 37 amino acids long. In some embodiments, the linker is 38 amino acids long. In some embodiments, the linker is 39 amino acids long. In some embodiments, the linker is 40 amino acids long. Exemplary linkers that may be used are Gly rich linkers, Gly and Ser containing linkers, Gly and Ala containing linkers, Ala and Ser containing linkers, and other flexible linkers.

Other linker sequences may include portions of immunoglobulin hinge area, CL or CH1 derived from any immunoglobulin heavy or light chain isotype. Exemplary linkers that may be used are shown in Table 4. Additional linkers are described for example in Int. Pat. Publ. No. WO2019/060695.

In some embodiments, the linker comprises the amino acid sequence of SEQ ID NO: 215.

In some embodiments, the linker comprises the amino acid sequence of SEQ ID NO: 216.

In some embodiments, the linker comprises the amino acid sequence of SEQ ID NO: 217.

In some embodiments, the linker comprises the amino acid sequence of SEQ ID NO: 218.

In some embodiments, the linker comprises the amino acid sequence of SEQ ID NO: 219.

In some embodiments, the linker comprises the amino acid sequence of SEQ ID NO: 220.

In some embodiments, the linker comprises the amino acid sequence of SEQ ID NO: 221.

In some embodiments, the linker comprises the amino acid sequence of SEQ ID NO: 222.

In some embodiments, the linker comprises the amino acid sequence of SEQ ID NO: 223.

In some embodiments, the linker comprises the amino acid sequence of SEQ ID NO: 224.

In some embodiments, the linker comprises the amino acid sequence of SEQ ID NO: 225.

In some embodiments, the linker comprises the amino acid sequence of SEQ ID NO: 226.

In some embodiments, the linker comprises the amino acid sequence of SEQ ID NO: 227.

In some embodiments, the linker comprises the amino acid sequence of SEQ ID NO: 228.

In some embodiments, the linker comprises the amino acid sequence of SEQ ID NO: 229.

In some embodiments, the linker comprises the amino acid sequence of SEQ ID NO: 230.

In some embodiments, the linker comprises the amino acid sequence of SEQ ID NO: 231.

In some embodiments, the linker comprises the amino acid sequence of SEQ ID NO: 232.

In some embodiments, the linker comprises the amino acid sequence of SEQ ID NO: 233.

In some embodiments, the linker comprises the amino acid sequence of SEQ ID NO: 234.

In some embodiments, the linker comprises the amino acid sequence of SEQ ID NO: 235.

In some embodiments, the linker comprises the amino acid sequence of SEQ ID NO: 236.

In some embodiments, the linker comprises the amino acid sequence of SEQ ID NO: 237.

In some embodiments, the linker comprises the amino acid sequence of SEQ ID NO: 238.

In some embodiments, the linker comprises the amino acid sequence of SEQ ID NO: 239.

In some embodiments, the linker comprises the amino acid sequence of SEQ ID NO: 240.

In some embodiments, the linker comprises the amino acid sequence of SEQ ID NO: 241.

In some embodiments, the linker comprises the amino acid sequence of SEQ ID NO: 242.

In some embodiments, the linker comprises the amino acid sequence of SEQ ID NO: 243.

In some embodiments, the linker comprises the amino acid sequence of SEQ ID NO: 244.

In some embodiments, the linker comprises the amino acid sequence of SEQ ID NO: 245.

In some embodiments, the linker comprises the amino acid sequence of SEQ ID NO: 246.

In some embodiments, the linker comprises the amino acid sequence of SEQ ID NO: 247.

In some embodiments, the linker comprises the amino acid sequence of SEQ ID NO: 248.

TABLE 4
Exemplary linker sequences

| | | SEQ |
|---|---|---|
| Linker | | ID |
| name | Amino acid sequence | NO: |
| Linker 1 | GGSEGKSSGSGSESKSTGGS | 215 |
| Linker 2 | GGGSGGGS | 216 |
| Linker 3 | GGGSGGGSGGGS | 217 |
| Linker 4 | GGGSGGGSGGGSGGGS | 218 |
| Linker 5 | GGGSGGGSGGGSGGGSGGGS | 219 |
| Linker 6 | GGGGSGGGGSGGGGS | 220 |
| Linker 7 | GGGGSGGGGSGGGGSGGGGS | 221 |
| Linker 8 | GGGGSGGGGSGGGGSGGGGSGGGGS | 222 |
| Linker 9 | GSTSGSGKPGSGEGSTKG | 223 |
| Linker 10 | IRPRAIGGSKPRVA | 224 |
| Linker 11 | GKGGSGKGGSGKGGS | 225 |
| Linker 12 | GGKGSGGKGSGGKGS | 226 |
| Linker 13 | GGGKSGGGKSGGGKS | 227 |
| Linker 14 | GKGKSGKGKSGKGKS | 228 |
| Linker 15 | GGGKSGGKGSGKGGS | 229 |
| Linker 16 | GKPGSGKPGSGKPGS | 230 |
| Linker 17 | GKPGSGKPGSGKPGSGKPGS | 231 |
| Linker 18 | GKGKSGKGKSGKGKSGKGKS | 232 |
| Linker 19 | STAGDTHLGGEDFD | 233 |
| Linker 20 | GEGGSGEGGSGEGGS | 234 |
| Linker 21 | GGEGSGGEGSGGEGS | 235 |
| Linker 22 | GEGESGEGESGEGES | 236 |
| Linker 23 | GGGESGGEGSGEGGS | 237 |
| Linker 24 | GEGESGEGESGEGESGEGES | 238 |
| Linker 25 | GSTSGSGKPGSGEGSTKG | 239 |
| Linker 26 | PRGASKSGSASQTGSAPGS | 240 |
| Linker 27 | GTAAAGAGAAGGAAAGAAG | 241 |
| Linker 28 | GTSGSSGSGSGGSGSGGGG | 242 |
| Linker 29 | GKPGSGKPGSGKPGSGKPGS | 243 |
| Linker 30 | GSGS | 244 |
| Linker 31 | APAPAPAPAP | 245 |
| Linker 32 | APAPAPAPAPAPAPAPAPAP | 246 |
| Linker 33 | AEAAAKEAAAKEAAAAKEAAAAKEAAAAKAAA | 247 |
| Linker 34 | GGGGSGGGGS | 248 |

In some embodiments, a trispecific antibody, or a trispecific antibody fragment of the present disclosure comprises a CD79b binding arm and a single polypeptide comprising the CD3 and CD2 binding arms (also referenced herein to as CD3/CD20 arm). The CD79b binding arm comprises a heavy chain portion (HC1), and a light chain (LC). The CD3/CD2 binding arm comprises from N to C terminus, a scFv that binds to CD3, heavy chain constant regions CH2 and CH3, and a scFv that binds to CD20. The trispecific molecule disclosed herein can be any one of the antibodies described in Table 5. In some embodiments, a trispecific antibody, or a trispecific antibody fragment of the present disclosure may be encoded by a nucleotide sequence encoding the CD79b arm and/or the CD3/CD20 arm of any one of the antibodies described in Table 5.

Table 5 provides a summary of examples of some CD79b x CD20 x CD3 trispecific antibodies described herein:

TABLE 5
Exemplary CD79b × CD20 × CD3 Trispecific antibodies

| | | | | | | | CD3/CD20 | |
|---|---|---|---|---|---|---|---|---|
| | | HC1 | | LC | | | arm | |
| | | Amino | HC1 | Amino | | | Amino | CD3/CD20 |
| | | acid | DNA | acid | LC DNA | | acid | arm DNA |
| | HC1/LC | sequence | sequence | sequence | sequence | | sequence | sequence |
| | (CD79b | SEQ ID | SEQ ID | SEQ ID | SEQ ID | CD3/CD20 | SEQ ID | SEQ ID |
| ID | arm) | NO | NO | NO | NO | arm | NO | NO |
| C923B38 | CD9B374 | 172 | 173 | 174 | 175 | CD3B2030- | 142 | 143 |
| | | | | | | N106A-scFv- | | |
| | | | | | | LH-C20B22 | | |
| C923B74 | CD9B330- | 176 | 177 | 178 | 179 | CD3B2030- | 142 | 143 |
| | N31S | | | | | N106A-scFv- | | |
| | | | | | | LH-C20B22 | | |
| C923B99 | CD9B643 | 180 | 181 | 182 | 183 | CD3B2030- | 142 | 143 |
| | | | | | | N106A-scFv- | | |
| | | | | | | LH-C20B22 | | |
| C923B36 | CD9B374 | 172 | 173 | 174 | 175 | CD3B2089- | 144 | 145 |
| | | | | | | N106G-scFv- | | |
| | | | | | | LH-C20B22 | | |
| C923B73 | CD9B330- | 176 | 177 | 178 | 179 | CD3B2089- | 144 | 145 |
| | N31S | | | | | N106G-scFv- | | |
| | | | | | | LH-C20B22 | | |
| C923B95 | CD9B643 | 180 | 181 | 182 | 183 | CD3B2089- | 144 | 145 |
| | | | | | | N106G-scFv- | | |
| | | | | | | LH-C20B22 | | |
| C923B138 | CD9B643 | 180 | 181 | 182 | 188 | CD3W245- | 148 | 149 |
| | | | | | | scFv LH- | | |
| | | | | | | C20B22 | | |
| C923B139 | CD9B643 | 180 | 181 | 182 | 188 | CD3B2089- | 150 | 151 |
| | | | | | | N106G-scFv | | |
| | | | | | | HL-C20B22 | | |
| C923B140 | CD9B643 | 180 | 181 | 182 | 188 | CD3W245- | 152 | 153 |
| | | | | | | scFv LH- | | |
| | | | | | | 5010GL | | |
| C923B141 | CD9B643 | 180 | 181 | 182 | 188 | CD3W245- | 154 | 155 |
| | | | | | | scFv LH- | | |
| | | | | | | 4A16GL | | |
| C923B142 | CD9B643 | 180 | 181 | 182 | 188 | CD3B2030- | 156 | 157 |
| | | | | | | N106A-LH | | |
| | | | | | | scFv- | | |
| | | | | | | 5010GL | | |
| C923B143 | CD9B643 | 180 | 181 | 182 | 188 | CD3B2030- | 158 | 159 |
| | | | | | | N106A-LH | | |
| | | | | | | scFv- | | |
| | | | | | | 4A16GL | | |
| C923B144 | CD9B643 | 180 | 181 | 182 | 188 | CD3B2089- | 160 | 161 |
| | | | | | | N106G-HL | | |
| | | | | | | scFv- | | |
| | | | | | | 5010GL | | |
| C923B145 | CD9B643 | 180 | 181 | 182 | 188 | CD3B2089- | 162 | 163 |
| | | | | | | N106G-HL | | |
| | | | | | | scFv- | | |
| | | | | | | 4A16GL | | |
| C923B147 | CD9B643 | 191 | 192 | 182 | 183 | CD3B2030- | 166 | 167 |
| | | | | | | N106A-LH | | |
| | | | | | | scFv- | | |
| | | | | | | 4A16GL | | |
| C923B168 | CD9B374 | 172 | 173 | 174 | 175 | CD3W245- | 168 | 169 |
| | | | | | | scFv LH- | | |
| | | | | | | C20B648 LH | | |
| C923B169 | CD9B374 | 172 | 173 | 174 | 175 | CD3B2030- | 170 | 171 |
| | | | | | | N106A-LH- | | |
| | | | | | | C20B648 LH | | |

Table 5 sets out the internal designation of each antigen-binding arm that is present in the various trispecific antibodies. The CDR sequences of CD79b arms are provided in Table 1; and the CDR sequences of CD3/CD20 arms are provided in Table 3 and Table 2a respectively. Thus, for example, trispecific antibody C923B 169 comprises CDRs 1, 2, 3, 4, 5, 6; 77, 76, 75, 79, 80, 81; 121, 122, 123, 124, 119 and 125.

In some embodiments, the HC1 of a CD79b x CD20 x CD3 trispecific antibody comprises an amino acid sequence substantially the same as, or identical to, SEQ ID NO: 172, 176, 180, or 191. In some embodiments, the nucleotide sequence encoding the HC1 of a CD79b x CD20 x CD3 trispecific antibody comprises the nucleotide sequence substantially the same as, or identical to, SEQ ID NO: 173, 177, 181, or 192.

In some embodiments, the LC of a CD79b x CD20 x CD3 trispecific antibody comprises an amino acid sequence substantially the same as, or identical to, SEQ ID NO: 174, 178 or 182. In some embodiments, the nucleotide sequence encoding the LC of a CD79b x CD20 x CD3 trispecific antibody comprises the nucleotide sequence substantially the same as, or identical to, SEQ ID NO: 175, 179, 183 or 188.

In some embodiments, the CD3/CD20 arm of a CD79b x CD20 x CD3 trispecific antibody comprises an amino acid sequence substantially the same as, or identical to, SEQ ID NO: 142, 144, 148, 150, 152, 154, 156, 158, 160, 162, 166, 168, or 170. In some embodiments, the nucleotide sequence encoding the CD3/CD20 arm of a CD79b x CD20 x CD3 trispecific antibody comprises the nucleotide sequence substantially the same as, or identical to, SEQ ID NO: 143, 145, 149, 151, 153, 155, 157, 159, 161, 163, 167, 169, or 171.

In some embodiments, the HC1 of a CD79b x CD20 x CD3 trispecific antibody comprises an amino acid sequence substantially the same as, or identical to, SEQ ID NO: 172 and the LC comprises the amino acid sequence substantially the same as, or identical to, SEQ ID NO: 174. In some embodiments, the nucleotide sequence encoding an HC1 of a CD79b x CD20 x CD3 trispecific antibody comprises the nucleotide sequence substantially the same as, or identical to, SEQ ID NO: 173 and the nucleotide sequence encoding an LC comprises the nucleotide sequence substantially the same as, or identical to, SEQ ID NO:175.

In some embodiments, the HC1 of a CD79b x CD20 x CD3 trispecific antibody comprises an amino acid sequence substantially the same as, or identical to, SEQ ID NO: 176 and the LC comprises an amino acid sequence substantially the same as, or identical to, SEQ ID NO: 178. In some embodiments, the nucleotide sequence encoding the HC1 of a CD79b x CD20 x CD3 trispecific antibody comprises the nucleotide sequence substantially the same as, or identical to, SEQ ID NO: 177 and the nucleotide sequence encoding the LC comprises the nucleotide sequence substantially the same as, or identical to, SEQ ID NO:179.

In some embodiments, the HC1 of a CD79b x CD20 x CD3 trispecific antibody comprises an amino acid sequence substantially the same as, or identical to, SEQ ID NO: 180 and the LC comprises an amino acid sequence substantially the same as, or identical to, SEQ ID NO: 182. In some embodiments, the nucleotide sequence encoding the HC1 of a CD79b x CD20 x CD3 trispecific antibody comprises the nucleotide sequence substantially the same as, or identical to, SEQ ID NO: 181 and the nucleotide sequence encoding the LC comprises the nucleotide sequence substantially the same as, or identical to, SEQ ID NO:183 or 188.

In some embodiments, the HC1 of a CD79b x CD20 x CD3 trispecific antibody comprises an amino acid sequence substantially the same as, or identical to, SEQ ID NO: 191 and the LC comprises an amino acid sequence substantially the same as, or identical to, SEQ ID NO: 182. In some embodiments, the nucleotide sequence encoding the HC1 of a CD79b x CD20 x CD3 trispecific antibody comprises the nucleotide sequence substantially the same as, or identical to, SEQ ID NO: 192 and the nucleotide sequence encoding the LC comprises the nucleotide sequence substantially the same as, or identical to, SEQ ID NO:183.

In some embodiments of a CD79b x CD20 x CD3 trispecific antibody, the HC1 comprises an amino acid sequence substantially the same as, or identical to, SEQ ID NO: 172, the LC comprises an amino acid sequence substantially the same as, or identical to, SEQ ID NO: 174, and the CD3/CD20 arm comprises an amino acid sequence substantially the same as, or identical to, SEQ ID NO: 142. In some embodiments, the nucleotide sequence encoding the HC1 comprises the nucleotide sequence substantially the same as, or identical to, SEQ ID NO: 173, the nucleotide sequence encoding the LC comprises the nucleotide sequence substantially the same as, or identical to, SEQ ID NO:175, and the nucleotide sequence encoding the CD3/CD20 arm comprises the nucleotide sequence substantially the same as, or identical to, SEQ ID NO: 143.

In some embodiments of a CD79b x CD20 x CD3 trispecific antibody, the HC1 comprises an amino acid sequence substantially the same as, or identical to, SEQ ID NO: 176, the LC comprises an amino acid sequence of substantially the same as, or identical to, SEQ ID NO: 178, and the CD3/CD20 arm comprises an amino acid sequence of substantially the same as, or identical to, SEQ ID NO: 142. In some embodiments, the nucleotide sequence encoding the HC1 comprises the nucleotide sequence substantially the same as, or identical to, SEQ ID NO: 177, the nucleotide sequence encoding the LC comprises the nucleotide sequence substantially the same as, or identical to, SEQ ID NO:179, and the nucleotide sequence encoding the CD3/CD20 arm comprises the nucleotide sequence substantially the same as, or identical to, SEQ ID NO: 143.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 142. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:183, and the nucleotide sequence encoding the CD3/CD20 arm comprises the nucleotide sequence substantially the same as, or identical to, SEQ ID NO: 143.

In some embodiments of a CD79b x CD20 x CD3 trispecific antibody, the HC1 comprises an amino acid sequence of substantially the same as, or identical to, SEQ ID NO: 172, the LC comprises an amino acid sequence of substantially the same as, or identical to, SEQ ID NO: 174, and the CD3/CD20 arm comprises an amino acid sequence of substantially the same as, or identical to, SEQ ID NO: 144. In some embodiments, the nucleotide sequence encoding the HC1 comprises the nucleotide sequence substantially the same as, or identical to, SEQ ID NO: 173, the nucleotide sequence encoding the LC comprises the nucleotide sequence substantially the same as, or identical to, SEQ ID NO:175 and the nucleotide sequence encoding the CD3/CD20 arm comprises the nucleotide sequence substantially the same as, or identical to, SEQ ID NO: 145.

In some embodiments of a CD79b x CD20 x CD3 trispecific antibody, the HC1 comprises an amino acid sequence of substantially the same as, or identical to, SEQ ID NO: 176, the LC comprises an amino acid sequence of substantially the same as, or identical to, SEQ ID NO: 178, and the CD3/CD20 arm comprises an amino acid sequence of substantially the same as, or identical to, SEQ ID NO: 144. In some embodiments, the nucleotide sequence encoding the HC1 comprises the nucleotide sequence substantially the same as, or identical to, SEQ ID NO: 177, the nucleotide sequence encoding the LC comprises the nucleotide sequence substantially the same as, or identical to, SEQ ID NO:179 and the nucleotide sequence encoding the CD3/CD20 arm comprises the nucleotide sequence substantially the same as, or identical to, SEQ ID NO: 145.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 144. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:183, and the nucleotide sequence encoding the CD3/CD20 arm comprises the nucleotide sequence substantially the same as, or identical to, SEQ ID NO: 145.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 148. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:183, and the nucleotide sequence encoding the CD3/CD20 arm comprises the nucleotide sequence substantially the same as, or identical to, SEQ ID NO: 149.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 150. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:188, and the nucleotide sequence encoding the CD3/CD20 arm comprises the nucleotide sequence substantially the same as, or identical to, SEQ ID NO: 151.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 152. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:188, and the nucleotide sequence encoding the CD3/CD20 arm comprises the nucleotide sequence substantially the same as, or identical to, SEQ ID NO: 153.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 154. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:188, and the nucleotide sequence encoding the CD3/CD20 arm comprises the nucleotide sequence substantially the same as, or identical to, SEQ ID NO: 155.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 156. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:188, and the nucleotide sequence encoding the CD3/CD20 arm comprises the nucleotide sequence substantially the same as, or identical to, SEQ ID NO: 157.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 158. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:188, and the nucleotide sequence encoding the CD3/CD20 arm comprises the nucleotide sequence substantially the same as, or identical to, SEQ ID NO: 159.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 160. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:188, and the nucleotide sequence encoding the CD3/CD20 arm comprises the nucleotide sequence substantially the same as, or identical to, SEQ ID NO: 161.

In some embodiments of a CD79b x CD20 x CD3 trispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3/CD20 arm comprises an amino acid sequence of substantially the same as, or identical to, SEQ ID NO: 162. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:188, and the nucleotide sequence encoding the CD3/CD20 arm comprises the nucleotide sequence substantially the same as, or identical to, SEQ ID NO: 163.

In some embodiments of a CD79b x CD20 x CD3 trispecific antibody, the HC1 comprises an amino acid sequence of substantially the same as, or identical to, SEQ ID NO: 191, the LC comprises an amino acid sequence of substantially the same as, or identical to, SEQ ID NO: 182, and the CD3/CD20 arm comprises an amino acid sequence of substantially the same as, or identical to, SEQ ID NO: 166. In some embodiments, the nucleotide sequence encoding the HC1 comprises the nucleotide sequence substantially the same as, or identical to, SEQ ID NO: 192, the nucleotide sequence encoding the LC comprises the nucleotide sequence substantially the same as, or identical to, SEQ ID NO:183, and the nucleotide sequence encoding the CD3/CD20 arm comprises the nucleotide sequence substantially the same as, or identical to, SEQ ID NO: 167.

In some embodiments of a CD79b x CD20 x CD3 trispecific antibody, the HC1 comprises an amino acid sequence of substantially the same as, or identical to, SEQ ID NO: 172, the LC comprises an amino acid sequence of substantially the same as, or identical to, SEQ ID NO: 174, and the CD3/CD20 arm comprises an amino acid sequence of substantially the same as, or identical to, SEQ ID NO: 168. In some embodiments, the nucleotide sequence encoding the HC1 comprises the nucleotide sequence substantially the same as, or identical to, SEQ ID NO: 173, the nucleotide sequence encoding the LC comprises the nucleotide sequence substantially the same as, or identical to, SEQ ID NO:175, and the nucleotide sequence encoding the CD3/CD20 arm comprises the nucleotide sequence substantially the same as, or identical to, SEQ ID NO: 169.

In some embodiments of a CD79b x CD20 x CD3 trispecific antibody, the HC1 comprises an amino acid sequence of substantially the same as, or identical to, SEQ ID NO: 172, the LC comprises an amino acid sequence of substantially the same as, or identical to, SEQ ID NO: 174, and the CD3/CD20 arm comprises an amino acid sequence of substantially the same as, or identical to, SEQ ID NO: 170. In some embodiments, the nucleotide sequence encoding the HC1 comprises the nucleotide sequence substantially the same as, or identical to, SEQ ID NO: 173, the nucleotide sequence encoding the LC comprises the nucleotide sequence substantially the same as, or identical to, SEQ ID NO:175 and the nucleotide sequence encoding the CD3/CD20 arm comprises the nucleotide sequence substantially the same as, or identical to, SEQ ID NO: 171.

In one embodiment, the HC1 of a CD79b x CD20 x CD3 trispecific antibody comprises an amino acid sequence substantially the same as, or identical to, SEQ ID NO: 172. In one embodiment, the HC1 of a CD79b x CD20 x CD3 trispecific antibody is encoded by a nucleotide sequence substantially the same as, or identical to, SEQ ID NO: 173.

In one embodiment, the LC of a CD79b x CD20 x CD3 trispecific antibody comprises an amino acid sequence substantially the same as, or identical to, SEQ ID NO: 174. In one embodiment, the LC of a CD79b x CD20 x CD3 trispecific antibody is encoded by a nucleotide sequence substantially the same as, or identical to, SEQ ID NO: 175.

In one embodiment, the CD3/CD20 arm of a CD79b x CD20 x CD3 trispecific antibody comprises an amino acid sequence substantially the same as, or identical to, SEQ ID NO: 168. In one embodiment, the CD3/CD20 arm of a CD79b x CD20 x CD3 trispecific antibody is encoded by a nucleotide sequence substantially the same as, or identical to, SEQ ID NO: 169.

In one embodiment, the CD3/CD20 arm of a CD79b x CD20 x CD3 trispecific antibody comprises an amino acid sequence substantially the same as, or identical to, SEQ ID NO: 170. In one embodiment, the CD3/CD20 arm of a CD79b x CD20 x CD3 trispecific antibody is encoded by a nucleotide sequence substantially the same as, or identical to, SEQ ID NO: 171.

In one embodiment, provided herein is an isolated trispecific antibody, or a trispecific binding fragment thereof, comprising:
a) a CD79b binding arm comprising a heavy chain (HC1) and a light chain (LC); and
b) a CD3/CD20 binding arm,
wherein HC1 comprises an amino acid sequence substantially the same as, or identical to, SEQ ID NO: 172, LC comprises an amino acid sequence substantially the same as, or identical to, SEQ ID NO: 174, and the CD3/CD20 binding arm comprises an amino acid sequence substantially the same as, or identical to, SEQ ID NO: 168.

In one embodiment, provided herein is an isolated trispecific antibody, or a trispecific binding fragment thereof, comprising:
a) a CD79b binding arm comprising a heavy chain (HC1) and a light chain (LC); and
b) a CD3/CD20 binding arm,
wherein HC1 comprises the amino acid sequence of SEQ ID NO: 172, LC comprises the amino acid sequence of SEQ ID NO: 174, and the CD3/CD20 binding arm comprises the amino acid sequence of SEQ ID NO: 168.

In one embodiment, provided herein is an isolated trispecific antibody, or a trispecific binding fragment thereof, comprising:
a) a CD79b binding arm comprising a heavy chain (HC1) and a light chain (LC); and
b) a CD3/CD20 binding arm,
wherein HC1 comprises an amino acid sequence substantially the same as, or identical to, SEQ ID NO: 172, LC comprises an amino acid sequence substantially the same as, or identical to, SEQ ID NO: 174, and the CD3/Cd20 binding arm comprises an amino acid sequence substantially the same as, or identical to, SEQ ID NO: 170.

In one embodiment, provided herein is an isolated trispecific antibody, or a trispecific binding fragment thereof, comprising:
a) a CD79b binding arm comprising a heavy chain (HC1) and a light chain (LC); and
b) a CD3/CD20 binding arm,
wherein HC1 comprises the amino acid sequence of SEQ ID NO: 172, LC comprises the amino acid sequence of SEQ ID NO: 174, and the CD3/CD20 binding arm comprises the amino acid sequence of SEQ ID NO: 170.

In one embodiment, the CD79b x CD20 x CD3 trispecific antibody is C923B168.

In one embodiment, the CD79b x CD20 x CD3 trispecific antibody is C923B169.

In some embodiments, a bispecific antibody, or a bispecific antibody fragment of the present disclosure comprises a CD79b binding arm comprising HC1 and LC, and/or CD3 binding arm of any one of the antibodies described in Table 6. In some embodiments, a bispecific antibody, or a bispecific antibody fragment of the present disclosure may be encoded by a nucleotide sequence encoding an HC1, LC, and/or CD3 binding arm of any one of the antibodies described in Table 6.

Table 6 provides a summary of examples of some CD79b x CD3 bispecific antibodies described herein:

TABLE 6
Exemplary CD79b × CD3 bispecific antibodies

| | | HC1 | | LC | | | CD3 arm | CD3 |
|---|---|---|---|---|---|---|---|---|
| | | amino | HC1 | amino | | | amino | arm |
| | | acid | DNA | acid | LC DNA | | acid | DNA |
| | HC1/LC | sequence | sequence | sequence | sequence | | sequence | sequence |
| | (CD79b | SEQ ID | SEQ ID | SEQ ID | SEQ ID | | SEQ ID | SEQ ID |
| ID | arm) | NO | NO | NO | NO | CD3-arm | NO | NO |
| 79C3B601 | CD9B374 | 172 | 173 | 174 | 175 | CD3B2030- | 164 | 165 |
| | | | | | | N106A | | |
| 79C3B646 | CD9B330- | 176 | 177 | 178 | 179 | CD3B2030- | 164 | 165 |
| | N31S | | | | | N106A | | |
| 79C3B651 | CD9B643 | 180 | 181 | 182 | 183 | CD3B2030- | 164 | 165 |
| | | | | | | N106A | | |
| 79C3B605 | CD9B374 | 172 | 173 | 174 | 175 | CD3B2089- | 189 | 190 |
| | | | | | | N106G | | |
| 79C3B645 | CD9B330- | 176 | 177 | 178 | 179 | CD3B2089- | 189 | 190 |
| | N31S | | | | | N106G | | |
| 79C3B650 | CD9B643 | 180 | 181 | 182 | 183 | CD3B2089- | 189 | 190 |
| | | | | | | N106G | | |

In some embodiments, the HC1 of a CD79b x CD3 bispecific antibody comprises an amino acid sequence substantially the same as, or identical to, SEQ ID NO: 172, 176, or 180. In some embodiments, the nucleotide sequence encoding an HC1 of a CD79b x CD3 bispecific antibody comprises the nucleotide sequence substantially the same as, or identical to, SEQ ID NO: 173, 177, or 181.

In some embodiments, the LC of a CD79b x CD3 bispecific antibody comprises an amino acid sequence substantially the same as, or identical to, SEQ ID NO: 174, 178, or 182. In some embodiments, the nucleotide sequence encoding an LC of a CD79b x CD3 bispecific antibody comprises the nucleotide sequence substantially the same as, or identical to, SEQ ID NO: 175, 179, or 183.

In some embodiments, the CD3 arm of a CD79b x CD3 bispecific antibody comprises an amino acid sequence substantially the same as, or identical to, SEQ ID NO: 164 or 189. In some embodiments, the nucleotide sequence encoding a CD3 arm of a CD79b x CD3 bispecific antibody comprises the nucleotide sequence substantially the same as, or identical to, SEQ ID NO: 165 or 190.

In some embodiments, the HC1 of a CD79b x CD3 bispecific antibody comprises an amino acid sequence substantially the same as, or identical to, SEQ ID NO: 172 and the LC comprises the amino acid sequence substantially the same as, or identical to, SEQ ID NO: 174. In some embodiments, the nucleotide sequence encoding an HC1 of a CD79b x CD3 bispecific antibody comprises the nucleotide sequence substantially the same as, or identical to, SEQ ID NO: 173 and the nucleotide sequence encoding an LC comprises the nucleotide sequence substantially the same as, or identical to, SEQ ID NO:175.

In some embodiments, the HC1 of a CD79b x CD3 bispecific antibody comprises an amino acid sequence substantially the same as, or identical to, SEQ ID NO: 176 and the LC comprises an amino acid sequence substantially the same as, or identical to, SEQ ID NO: 178. In some embodiments, the nucleotide sequence encoding an HC1 of a CD79b x CD3 bispecific antibody comprises the nucleotide sequence substantially the same as, or identical to, SEQ ID NO: 177 and the nucleotide sequence encoding an LC comprises the nucleotide sequence substantially the same as, or identical to, SEQ ID NO:179.

In some embodiments, the HC1 of a CD79b x CD3 bispecific antibody comprises an amino acid sequence substantially the same as, or identical to, SEQ ID NO: 180 and the LC comprises an amino acid sequence substantially the same as, or identical to, SEQ ID NO: 182. In some embodiments, the nucleotide sequence encoding an HC1 of a CD79b x CD3 bispecific antibody comprises the nucleotide sequence substantially the same as, or identical to, SEQ ID NO: 181 and the nucleotide sequence encoding an LC comprises the nucleotide sequence substantially the same as, or identical to, SEQ ID NO:183 or 188.

In some embodiments of a CD79b x CD3 bispecific antibody, the HC1 comprises an amino acid sequence substantially the same as, or identical to, SEQ ID NO: 172, the LC comprises an amino acid sequence substantially the same as, or identical to, SEQ ID NO: 174, and the CD3 arm comprises an amino acid sequence substantially the same as, or identical to, SEQ ID NO: 164. In some embodiments, the nucleotide sequence encoding the HC1 comprises the nucleotide sequence substantially the same as, or identical to, SEQ ID NO: 173, the nucleotide sequence encoding the LC comprises the nucleotide sequence substantially the same as, or identical to, SEQ ID NO:175 and the nucleotide sequence encoding the CD3 arm comprises the nucleotide sequence substantially the same as, or identical to, SEQ ID NO: 165.

In some embodiments of a CD79b x CD3 bispecific antibody, the HC1 comprises an amino acid sequence of substantially the same as, or identical to, SEQ ID NO: 176, the LC comprises an amino acid sequence of substantially the same as, or identical to, SEQ ID NO: 178, and the CD3 arm comprises an amino acid sequence of substantially the same as, or identical to, SEQ ID NO: 164. In some embodiments, the nucleotide sequence encoding the HC1 comprises the nucleotide sequence substantially the same as, or identical to, SEQ ID NO: 177, the nucleotide sequence encoding the LC comprises the nucleotide sequence substantially the same as, or identical to, SEQ ID NO:179, and the nucleotide sequence encoding the CD3 arm comprises the nucleotide sequence substantially the same as, or identical to, SEQ ID NO: 165.

In some embodiments of a CD79b x CD3 bispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3 arm comprises an amino acid sequence of substantially the same as, or identical to, SEQ ID NO: 164. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:183, and the nucleotide sequence encoding the CD3 arm comprises the nucleotide sequence substantially the same as, or identical to, SEQ ID NO: 165.

In some embodiments of a CD79b x CD3 bispecific antibody, the HC1 comprises an amino acid sequence of substantially the same as, or identical to, SEQ ID NO: 172, the LC comprises an amino acid sequence of substantially the same as, or identical to, SEQ ID NO: 174, and the CD3 arm comprises an amino acid sequence of substantially the same as, or identical to, SEQ ID NO: 189. In some embodiments, the nucleotide sequence encoding the HC1 comprises the nucleotide sequence substantially the same as, or identical to, SEQ ID NO: 173, the nucleotide sequence encoding the LC comprises the nucleotide sequence substantially the same as, or identical to, SEQ ID NO:175, and the nucleotide sequence encoding the CD3 arm comprises the nucleotide sequence substantially the same as, or identical to, SEQ ID NO: 190.

In some embodiments of a CD79b x CD3 bispecific antibody, the HC1 comprises an amino acid sequence of substantially the same as, or identical to, SEQ ID NO: 176, the LC comprises an amino acid sequence of substantially the same as, or identical to, SEQ ID NO: 178, and the CD3 arm comprises an amino acid sequence of substantially the same as, or identical to, SEQ ID NO: 189. In some embodiments, the nucleotide sequence encoding the HC1 comprises the nucleotide sequence substantially the same as, or identical to, SEQ ID NO: 177, the nucleotide sequence encoding the LC comprises the nucleotide sequence substantially the same as, or identical to, SEQ ID NO:179, and the nucleotide sequence encoding the CD3 arm comprises the nucleotide sequence substantially the same as, or identical to, SEQ ID NO: 190.

In some embodiments of a CD79b x CD3 bispecific antibody, the HC1 comprises an amino acid sequence of substantially the same as, or identical to, SEQ ID NO: 180, the LC comprises an amino acid sequence of substantially the same as, or identical to, SEQ ID NO: 182, and the CD3 arm comprises an amino acid sequence of substantially the same as, or identical to, SEQ ID NO: 189. In some embodiments, the nucleotide sequence encoding the HC1 comprises the nucleotide sequence substantially the same as, or identical to, SEQ ID NO: 181, the nucleotide sequence encoding the LC comprises the nucleotide sequence substantially the same as, or identical to, SEQ ID NO:183, and the nucleotide sequence encoding the CD3 arm comprises the nucleotide sequence substantially the same as, or identical to, SEQ ID NO: 190.

In addition to the described multispecific antibodies or antigen-binding fragments, also provided are polynucleotide sequences capable of encoding the described multispecific antibodies or antigen-binding fragments. Vectors comprising the described polynucleotides are also provided, as are cells expressing the multispecific antibodies or antigen-binding fragments provided herein. Also described are cells capable of expressing the disclosed vectors. These cells may be mammalian cells (such as 293F cells, CHO cells), insect cells (such as Sf7 cells), yeast cells, plant cells, or bacteria cells (such as E. coli). The described antibodies may also be produced by hybridoma cells. The described antibodies may also be recombinantly produced.

Polynucleotides encoding recombinant antigen-binding proteins also are within the scope of the disclosure. In some embodiments, the polynucleotides described (and the peptides they encode) include a leader sequence. Any leader sequence known in the art may be employed. The leader sequence may include, but is not limited to, a restriction site or a translation start site.

The multispecific antibodies or antigen-binding fragments described herein include variants having single or multiple amino acid substitutions, deletions, or additions that retain the biological properties (e.g., binding affinity or immune effector activity) of the described multispecific antibodies or antigen-binding fragments. In the context of the present invention the following notations are, unless otherwise indicated, used to describe a mutation; i) substitution of an amino acid in a given position is written as e.g. K409R which means a substitution of a Lysine in position 409 with an Arginine; and ii) for specific variants the specific three or one letter codes are used, including the codes Xaa and X to indicate any amino acid residue. Thus, the substitution of Arginine for Lysine in position 409 is designated as: K409R, or the substitution of any amino acid residue for Lysine in position 409 is designated as K409X. In case of deletion of Lysine in position 409 it is indicated by K409*. The skilled person may produce variants having single or multiple amino acid substitutions, deletions, or additions.

These variants may include: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Antibodies or antigen-binding fragments described herein may include variants in which amino acid residues from one species are substituted for the corresponding residue in another species, either at the conserved or nonconserved positions. In other embodiments, amino acid residues at nonconserved positions are substituted with conservative or nonconservative residues. The techniques for obtaining these variants, including genetic (deletions, mutations, etc.), chemical, and enzymatic techniques, are known to persons having ordinary skill in the art.

The multispecific antibodies or antigen-binding fragments described herein may embody several antibody isotypes, such as IgM, IgD, IgG, IgA and IgE. In some embodiments the antibody isotype is IgG1, IgG2, IgG3, or IgG4 isotype, preferably IgG1 or IgG4 isotype. Antibody or antigen-binding fragment thereof specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

Also provided are vectors comprising the polynucleotides described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors.

Recombinant expression vectors within the scope of the description include synthetic, genomic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the multispecific antibody- or antigen-binding fragment-coding sequence is placed under control of a powerful constitutive promoter, such as the promoters for the following genes: hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include without limitation, Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. In one embodiment, the multispecific antibody or antigen-binding fragment thereof coding sequence is placed under control of an inducible promoter such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene, a puromycin resistance gene, a blasticidin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate multispecific antibody or antigen-binding fragment-producing cells. Thus, another aspect features host cells transformed with vectors comprising a nucleic acid sequence encoding an antibody or antigen-binding fragment thereof that binds CD79b, CD20, and/or CD3, such as the antibodies or antigen-binding fragments described and exemplified herein.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Cells suitable for use in the expression of the multispecific antibodies or antigen-binding fragments described herein are preferably eukaryotic cells, more preferably cells of plant, rodent, or human origin, for example but not limited to NSO, CHO, CHOK1, perC.6, Tk-ts13, BHK, HEK293 cells, COS-7, T98G, CV-1/EBNA, L cells, C127, 3T3, HeLa, NS1, Sp2/0 myeloma cells, and BHK cell lines, among others. In addition, expression of antibodies may be accomplished using hybridoma cells. Methods for producing hybridomas are well established in the art.

Cells transformed with expression vectors described herein may be selected or screened for recombinant expression of the antibodies or antigen-binding fragments described herein. Recombinant-positive cells are expanded and screened for subclones exhibiting a desired phenotype, such as high level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

### Therapeutic Composition and Methods of Treatment Using Multispecific Antibodies and Multispecific Antigen-Binding Fragments Thereof

The multispecific antibodies discussed above, for example the CD79b x CD20 x CD3 trispecific antibodies or the CD79b x CD3 bispecific antibodies discussed above, are useful in therapy. In particular, the multispecific antibodies are useful in treating cancer. Also provided herein are therapeutic compositions for the treatment of a hyperproliferative disorder in a mammal which comprises a therapeutically effective amount of a multispecific antibody or multispecific antigen-binding fragment described herein and a pharmaceutically acceptable carrier. In some embodiments, the multispecific antibody is a CD79b x CD20 x CD3 trispecific antibody as described herein, or a CD79b x CD20 x CD3-trispecific antigen-binding fragment thereof. In some embodiments, the bispecific antibody is a CD79b x CD3 trispecific antibody as described herein, or a CD79b x CD3-bispecific antigen-binding fragment thereof. In one embodiment said pharmaceutical composition is for the treatment of a CD79b and/or CD20-expressing cancer, including (but not limited to) the following: CD79b and/or CD20-expressing B cell cancers, such as B-cell lymphoma, non-Hodgkin lymphoma, diffuse large B-cell lymphoma (DLBCL), a mantle cell lymphoma (MCL), a follicular lymphoma (FL), acute lymphoblastic leukemia (ALL), chronic lymphocytic leukemia (CLL), Waldenström macroglobulinemia (WM), multiple myeloma (MM), mucosa-associated lymphoid tissue (MALT) lymphoma, Hodgkin's lymphoma, Burkitt's lymphoma, hairy-cell leukemia, or Plasmacytoma cells, and other cancers yet to be determined in which CD79b and/or CD20 is expressed. Particular trispecific antibodies that may be used to treat cancer, such as hematological cancer, including the specific cancers discussed above, include antibodies C923B168, and C923B169.

In some embodiments, the CD79b x CD20 x CD3 trispecific antibody or binding fragment thereof is utilized for the treatment of diffuse large B-cell lymphoma (DLBCL) including R/R DLBCL.

In some embodiments, the CD79b x CD20 x CD3 trispecific antibody or binding fragment thereof is utilized for the treatment of non-Hodgkin lymphoma (including R/R non-Hodgkin lymphoma) such as follicular lymphoma (FL) or marginal zone lymphoma (MZL).

In some embodiments, the CD79b x CD20 x CD3 trispecific antibody or binding fragment thereof is utilized for the treatment of mantle cell lymphoma (MCL), including R/R MCL.

In some embodiments, the CD79b x CD20 x CD3 trispecific antibody or binding fragment thereof is utilized for the treatment of chronic lymphocytic leukemia (CLL), including R/R CLL.

In some embodiments, the CD79b x CD20 x CD3 trispecific antibody or binding fragment thereof is utilized for the treatment of Waldenström macroglobulinemia (WM), including R/R WM.

In some embodiments, the CD79b x CD20 x CD3 trispecific antibody or binding fragment thereof is utilized for the treatment Burkitt lymphoma (BL).

In some embodiments, the CD79b x CD20 x CD3 trispecific antibody or binding fragment thereof is utilized for the treatment of primary mediastinal b cell lymphoma (PMBCL).

In some embodiments, the CD79b x CD20 x CD3 trispecific antibody or binding fragment thereof is utilized for the treatment of nodular lymphomcyte predominant Hodgkin Lymphoma (nLPHL).

In some embodiments, the CD79b x CD20 x CD3 trispecific antibody or binding fragment thereof is utilized for the treatment of post-transplantation lymphoproliferative disorders (PTLD).

In some embodiments, the CD79b x CD20 x CD3 trispecific antibody or binding fragment thereof is utilized for the treatment of primary central nervous system lymphoma (PCNSL).

In some embodiments, the CD79b x CD20 x CD3 trispecific antibody or binding fragment thereof is utilized for autologous and allogeneic stem cell transplantation (autoSCT and alloSCT) as a condition regimen, and as a chemotherapy-free maintenance therapy or proactive prevention of relapse post-SCT with the intention of curing relapsed B-cell lymphoma.

In some embodiments, the CD79b x CD20 x CD3 trispecific antibody or binding fragment thereof is utilized for local or topical treatment of cutaneous B-cell lymphoma (including DLBCL Leg type), bronchus-associated lymphoid tissue (BALT) lymphoma, intraocular lymphoma (IOL) including primary intraocular lymphoma (PIOL) or secondary intraocular lymphoma (SIOL) of the B-cell lineage. In these embodiments, the CD79b x CD20 x CD3 trispecific antibody or binding fragment thereof may utilize new administration approaches, such as inhaler or nebulizer for BALT lymphoma affecting the lungs.

The pharmaceutical compositions provided herein comprise: a) an effective amount of a multispecific antibody or antibody fragment of the present invention, and b) a pharmaceutically acceptable carrier, which may be inert or physiologically active. In some embodiments, the multispecific antibody is a CD79b x CD20 x CD3 trispecific antibody as described herein, or a CD79b x CD20 x CD3-trispecific antigen-binding fragment thereof. In some embodiments, the bispecific antibody is a CD79b x CD3 trispecific antibody as described herein, or a CD79b x CD3-bispecific antigen-binding fragment thereof. As used herein, the term “pharmaceutically acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as any combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH. about.7.4, containing or not containing about 1 mg/mL to 25 mg/mL human serum albumin, (2) 0.9% saline (0.9% w/v sodium chloride (NaCl)), and (3) 5% (w/v) dextrose; and may also contain an antioxidant such as tryptamine and a stabilizing agent such as Tween 20®.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperitoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the antibody, antibody fragment or antibody conjugate of the present invention in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohol's, or sodium chloride in the composition. These compositions may also contain adjuvants, in particular wetting, isotonizing, emulsifying, dispersing and stabilizing agents. Sterile compositions for parenteral administration may also be prepared in the form of sterile solid compositions which may be dissolved at the time of use in sterile water or any other injectable sterile medium.

The multispecific antibody or antibody fragment may also be orally administered. As solid compositions for oral administration, tablets, pills, powders (gelatin capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used; they are generally between 5 mg and 1000 mg per day orally for an adult with unit doses ranging from 1 mg to 250 mg of active substance. In general, the doctor will determine the appropriate dosage depending on the age, weight and any other factors specific to the subject to be treated.

Also provided herein are methods for killing a CD79b and/or CD20+ cell by administering to a patient in need thereof a multispecific antibody which binds said CD79b and/or CD20 and is able to recruit T cells to kill said CD79b and/or CD20+ cell (i.e., T cell redirection). Any of the multispecific antibodies or antibody fragments of the invention may be used therapeutically. For example, in one embodiment the CD79b x CD20 x CD3-multispecific antibody may be used therapeutically to treat cancer in a subject.

In a preferred embodiment, multispecific antibodies or antibody fragments of the invention are used for the treatment of a hyperproliferative disorder in a mammal. In a more preferred embodiment, one of the pharmaceutical compositions disclosed above, and which contains a multispecific antibody or antibody fragment of the invention, is used for the treatment of a hyperproliferative disorder in a mammal. In one embodiment, the disorder is a cancer. In particular, the cancer is a CD79b and/or CD20-expressing cancer, including (but not limited to) the following: CD79b and/or CD20-expressing B-cell cancers, such as B-cell lymphoma, non-Hodgkin lymphoma, diffuse large B-cell lymphoma (DLBCL), a mantle cell lymphoma (MCL), a follicular lymphoma (FL), acute lymphoblastic leukemia (ALL), chronic lymphocytic leukemia (CLL), Waldenström macroglobulinemia (WM), multiple myeloma (MM), mucosa-associated lymphoid tissue (MALT) lymphoma, Hodgkin's lymphoma, Burkitt's lymphoma, hairy-cell leukemia, or Plasmacytoma cells, and other cancers yet to be determined in which CD79b and/or CD20 is expressed. In some embodiments, the CD79b and/or CD20-expressing B-cell cancer treated with the pharmaceutical composition disclosed herein is a relapsed or refractory form of the cancer. In preferred embodiments, the multispecific antibody is a CD79b x CD20 x CD3-multispecific antibody as described herein, or a multispecific antigen-binding fragment thereof, and more preferably a CD79b x CD20 x CD3-trispecific antibody as described herein, or a CD79b x CD20 x CD3-trispecific antigen-binding fragment thereof.

Accordingly, the pharmaceutical compositions of the invention are useful in the treatment or prevention of a variety of cancers, including (but not limited to) the following: a CD79b and/or CD20-expressing cancer, including (but not limited to) the following: CD79b and/or CD20-expressing B-cell cancers, such as B-cell lymphoma, non-Hodgkin lymphoma, diffuse large B-cell lymphoma (DLBCL), a mantle cell lymphoma (MCL), a follicular lymphoma (FL), acute lymphoblastic leukemia (ALL), chronic lymphocytic leukemia (CLL), Waldenström macroglobulinemia (WM), multiple myeloma (MM), mucosa-associated lymphoid tissue (MALT) lymphoma, Hodgkin's lymphoma, Burkitt's lymphoma, hairy-cell leukemia, or Plasmacytoma cells, and other cancers yet to be determined in which CD79b and/or CD20 is expressed. In some embodiments, the CD79b and/or CD20-expressing B-cell cancer treated with the pharmaceutical composition disclosed herein is a relapsed or refractory form of the cancer.

Similarly, further provided herein is a method for inhibiting the growth of selected cell populations comprising contacting CD79b and/or CD20-expressing target cells, or tissue containing such target cells, with an effective amount of a multispecific antibody or antibody fragment of the present invention, either alone or in combination with other cytotoxic or therapeutic agents, in the presence of a peripheral blood mononuclear cell (PBMC). A CD79b x CD20 x CD3 antibody that blocks the binding of ligands to CD79b and CD20 may block CD79b- and CD20-mediated signaling and lead to inhibition or cell death of the target cells. In preferred embodiments, the multispecific antibody is a CD79b x CD20 x CD3-multispecific antibody as described herein, or a multispecific antigen-binding fragment thereof, and more preferably a CD79b x CD20 x CD3-trispecific antibody as described herein, or a CD79b x CD20 x CD3-trispecific antigen-binding fragment thereof.

In some embodiments, the methods described herein involving the administration of a multispecific antibody or pharmaceutical composition comprising the same, further involve administering another therapeutic agent. Suitable other therapeutic agents include, without limitation, a chemotherapeutic agent, an anti-CD20 agent, an anti-CD19 agent, an anti-CD22 agent, an anti-CD3γ agent, a Bruton's tyrosine kinase (BTK) inhibitor, a mucosa-associated lymphoid tissue lymphoma translocation protein 1 (MALT1) inhibitor, an immunomodulatory imide drug (IMiD), a pro apoptotic B cell lymphoma 2 (Bcl-2) family inhibitor, a phosphoinositide 3-kinase (PI3K) inhibitor, a NFKB-inducing kinase (NIK) inhibitor, an immune checkpoint inhibitor, a CD28 costimulatory bispecific antibody, or a CD137 costimulatory bispecific antibody, or a combination thereof.

In some embodiments, the other therapeutic agent is a chemotherapeutic regimen, such as rituximab-cyclophosphamide-hydroxydaunorubicin-oncovin-prednisone/prednisolone (R-CHOP), rituximab-ifosfamide-carboplatin-etoposide (R-ICE), rituximab-(dose-adjusted) etoposide-prednisolone-oncovin-cyclophosphamide-hydroxydaunorubicin (R [DA]EPOCH), rituximab-dexamethasone-high-dose ara C cytarabine-platinol (R-DHAP), rituximab-etoposide-solu-medrol-high-dose ara C cytarabine-platinol (R-ESHAP), bendamustine-rituximab (BR), and lenalidomide+rituximab (R²), Polivy (polatuzumab vedotin)+BR, or Monjuvi (tafasitamab-cxix)+lenalidomide regimens.

In some embodiments, the other therapeutic agent is an anti-CD79b agent (e.g. an anti-CD79b CAR-T therapy), anti-CD20 agent, such as a CD20 x CD3 bispecific antibody (e.g., mosunetuzumab, glofitamab, odronextamab, IGM-2323, and epcoritamab). In some embodiments, the other therapeutic agent is an anti-CD19 agent, such as an anti-CD19 antibody (e.g., tafasitamab), an anti-CD19 CAR-T therapy (e.g., Yescarta, Kymriah, and Breyanzi), or an CD20 x CD3 bispecific antibody. In some embodiments, the other therapeutic agent is an anti-CD22 agent, such as anti-CD22 antibody (e.g., moxetumomab pasudotox). In some embodiments, the other therapeutic agent is a Bruton's tyrosine kinase (BTK) inhibitor such as ibrutinib, covalent or non-covalent BTK inhibitors.

In some embodiments, the other therapeutic agent is a mucosa-associated lymphoid tissue lymphoma translocation protein 1 (MALT1) inhibitor. In some embodiments, the other therapeutic agent is an immunomodulatory imide drug (IMiD), such as lenalidominde. In some embodiments, the other therapeutic agent is a pro apoptotic B cell lymphoma 2 (Bcl-2) family inhibitor such as venetoclax and a Bcl-2-related protein A1 (BFL-1) inhibitor. In some embodiments, the other therapeutic agent is a phosphoinositide 3-kinase (PI3K) inhibitor, such as parsaclisib, idelalisib and umbralisib. In some embodiments, the other therapeutic agent is an immune checkpoint inhibitor, such as an anti-PD 1 antibody, anti T-cell Ig, TIM-3, T cell immunoreceptor with Ig and ITIM domains [TIGIT], and LAG-3. In some embodiments, the further therapeutic agent is cytarabine, an anthracycline, histamine dihydrochloride, selinexor, tasemetostat or interleukin 2. In some embodiments, the further therapeutic agent is a chemotherapeutic agent. The method for inhibiting the growth of selected cell populations can be practiced in vitro, in vivo, or ex vivo.

Examples of in vitro uses include treatments of autologous bone marrow prior to their transplant into the same patient in order to kill diseased or malignant cells; and prevent graft-versus-host-disease (GVHD); treatments of cell cultures in order to kill all cells except for desired variants that do not express the target antigen; or to kill variants that express undesired antigen. The conditions of non-clinical in vitro use are readily determined by one of ordinary skill in the art.

Examples of clinical ex vivo use are to remove tumor cells from bone marrow prior to autologous transplantation in cancer treatment. Treatment can be carried out as follows. Bone marrow is harvested from the patient or other individual, or a provided sample is used, and then incubated in medium containing serum to which is added the cytotoxic agent of the invention. Concentrations range from about 10 μM to 1 μM, for about 30 min to about 48 hr at about 37° C. The exact conditions of concentration and time of incubation, i.e., the dose, are readily determined by one of ordinary skill in the art. After incubation the bone marrow cells are washed with medium containing serum and returned to the patient by i.v. infusion according to known methods. In circumstances where the patient receives other treatment such as a course of ablative chemotherapy or total-body irradiation between the time of harvest of the marrow and reinfusion of the treated cells, the treated marrow cells are stored frozen in liquid nitrogen using standard medical equipment.

For clinical in vivo use, a therapeutically effective amount of the multispecific antibody or antigen-binding fragment is administered to a subject in need thereof. For example, the CD79b x CD20 x CD3-multispecific antibodies and multispecific antigen-binding fragments thereof may be useful in the treatment of a CD79b and/or CD20-expressing cancer in a subject in need thereof. In some embodiments, the CD79b and/or CD20-expressing cancer is a B-cell cancer, such as diffuse large B-cell lymphoma (DLBCL). In preferred embodiments, the multispecific antibody is a CD79b x CD20 x CD3-multispecific antibody as described herein, or a multispecific antigen-binding fragment thereof, and more preferably a CD79b x CD20 x CD3-trispecific antibody as described herein, or a CD79b x CD20 x CD3-trispecific antigen-binding fragment thereof. In some embodiments, the subject is a mammal, preferably a human. In some embodiments, the multispecific antibody or antigen-binding fragment will be administered as a solution that has been tested for sterility.

Dosage regimens in the above methods of treatment and uses are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage.

The efficient dosages and the dosage regimens for the multispecific antibodies and fragments depend on the disease or condition to be treated and may be determined by one skilled in the art. An exemplary, non-limiting range for a therapeutically effective amount of a compound of the present invention is about 0.001-10 mg/kg, such as about 0.001-5 mg/kg, for example about 0.001-2 mg/kg, such as about 0.001-1 mg/kg, for instance about 0.001, about 0.01, about 0.1, about 1 or about 10 mg/kg.

A physician or veterinarian having ordinary skill in the art may readily determine and prescribe the effective amount of the pharmaceutical composition required. For example, the physician or veterinarian could start doses of the multispecific antibody or fragment employed in the pharmaceutical composition at levels lower than that required in order to achieve the desired therapeutic effect and gradually increase the dosage until the desired effect is achieved. In general, a suitable daily dose of a multispecific antibody of the present invention will be that amount of the compound which is the lowest dose effective to produce a therapeutic effect. Administration may e.g. be parenteral, such as intravenous, intramuscular, intratumoral (e.g., bone marrow) or subcutaneous. In one embodiment, the multispecific antibody or fragment may be administered by infusion in a weekly dosage of calculated by mg/m². Such dosages can, for example, be based on the mg/kg dosages provided above according to the following: dose (mg/kg)×body weight (e.g., 50-100 kg). Such administration may be repeated, e.g., 1 to 8 times, such as 3 to 5 times. The administration may be performed by continuous infusion over a period of from 2 to 24 hr, such as of from 2 to 12 hr. In one embodiment, the multispecific antibody or fragment may be administered by slow continuous infusion over a long period, such as more than 24 hours, in order to reduce toxic side effects.

In one embodiment, the multispecific antibody or fragment may be administered in a weekly dosage of calculated as a fixed dose for up to eight times, such as from four to six times when given once a week. Such regimen may be repeated one or more times as necessary, for example, after six months or twelve months. Such fixed dosages can, for example, be based on the mg/kg dosages provided above, with a body weight estimate of 50-100 kg. The dosage may be determined or adjusted by measuring the amount of multispecific antibody of the present invention in the blood upon administration by for instance taking out a biological sample and using anti-idiotypic antibodies which target the CD79b and/or CD20 antigen binding arms of the multispecific antibodies of the present invention.

In one embodiment, the multispecific antibody or fragment may be administered by maintenance therapy, such as, e.g., once a week for a period of six months or more.

A multispecific antibody or fragment may also be administered prophylactically in order to reduce the risk of developing cancer, delay the onset of the occurrence of an event in cancer progression, and/or reduce the risk of recurrence when a cancer is in remission.

The multispecific antibodies and fragments thereof as described herein may also be administered in combination therapy, i.e., combined with other therapeutic agents relevant for the disease or condition to be treated. Accordingly, in one embodiment, the antibody-containing medicament is for combination with one or more further therapeutic agent, such as a chemotherapeutic agent, an anti-CD20 agent, an anti-CD19 agent, an anti-CD22 agent, an anti-CD37 agent, a Bruton's tyrosine kinase (BTK) inhibitor, a mucosa-associated lymphoid tissue lymphoma translocation protein 1 (MALT1) inhibitor, an immunomodulatory imide drug (IMiD), a pro apoptotic B cell lymphoma 2 (Bcl-2) family inhibitor, a phosphoinositide 3-kinase (PI3K) inhibitor, a NFKB-inducing kinase (NIK) inhibitor, an immune checkpoint inhibitor, a CD28 costimulatory bispecific antibody, or a CD137 costimulatory bispecific antibody, or a combination thereof. In some embodiments, the other therapeutic agent is a chemotherapeutic regimen, such as rituximab-cyclophosphamide-hydroxydaunorubicin-oncovin-prednisone/prednisolone (R-CHOP), rituximab-ifosfamide-carboplatin-etoposide (R-ICE), rituximab-(dose-adjusted) etoposide-prednisolone-oncovin-cyclophosphamide-hydroxydaunorubicin (R [DA]EPOCH), rituximab-dexamethasone-high-dose ara C cytarabine-platinol (R-DHAP), rituximab-etoposide-solu-medrol-high-dose ara C cytarabine-platinol (R-ESHAP), bendamustine-rituximab (BR), and lenalidomide+rituximab (R²), Polivy (polatuzumab vedotin)+BR, or Monjuvi (tafasitamab-cxix)+lenalidomide regimens. In some embodiments, the other therapeutic agent is an anti-CD79b agent (e.g. an anti-CD79b CAR-T therapy), anti-CD20 agent, such as a CD20xCD3 bispecific antibody (e.g., mosunetuzumab, glofitamab, odronextamab, IGM-2323, and epcoritamab). In some embodiments, the other therapeutic agent is an anti-CD19 agent, such as an anti-CD19 antibody (e.g., tafasitamab), an anti-CD19 CAR-T therapy (e.g., Yescarta, Kymriah, and Breyanzi), or an CD20xCD3 bispecific antibody. In some embodiments, the other therapeutic agent is an anti-CD22 agent, such as anti-CD22 antibody (e.g., moxetumomab pasudotox). In some embodiments, the other therapeutic agent is a Bruton's tyrosine kinase (BTK) inhibitor such as ibrutinib, covalent or non-covalent BTK inhibitors. In some embodiments, the other therapeutic agent is a mucosa-associated lymphoid tissue lymphoma translocation protein 1 (MALT1) inhibitor. In some embodiments, the other therapeutic agent is an immunomodulatory imide drug (IMiD), such as lenalidominde. In some embodiments, the other therapeutic agent is a pro apoptotic B cell lymphoma 2 (Bcl-2) family inhibitor such as venetoclax and a Bcl-2-related protein A1 (BFL-1) inhibitor. In some embodiments, the other therapeutic agent is a phosphoinositide 3-kinase (PI3K) inhibitor, such as parsaclisib, idelalisib and umbralisib. In some embodiments, the other therapeutic agent is an immune checkpoint inhibitor, such as an anti-PD 1 antibody, anti T-cell Ig, TIM-3, T cell immunoreceptor with Ig and ITIM domains [TIGIT], and LAG-3. In some embodiments, the other therapeutic agent is cytarabine, an anthracycline, histamine dihydrochloride, selinexor, tasemetostat or interleukin 2. Such combined administration may be simultaneous, separate or sequential, in any order. For simultaneous administration the agents may be administered as one composition or as separate compositions, as appropriate.

In one embodiment, a method for treating a disorder involving cells expressing CD79b and/or CD20 in a subject, which method comprises administration of a therapeutically effective amount of a multispecific antibody or fragment, such as a CD79b x CD20 x CD3 multispecific antibody described herein, and radiotherapy to a subject in need thereof is provided. In one embodiment is provided a method for treating or preventing cancer, which method comprises administration of a therapeutically effective amount of a multispecific antibody or fragment, such as a CD79b x CD20 x CD3 antibody described herein, and radiotherapy to a subject in need thereof. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, actinium-225, and indium-111.

### Kits

Also provided herein are includes kits, e.g., comprising a described multispecific antibody or antigen-binding fragment thereof and instructions for the use of the antibody or fragments for killing of particular cell types. In preferred embodiments, the multispecific antibody is a CD79b x CD20 x CD3-multispecific antibody as described herein, or a multispecific antigen-binding fragment thereof, and more preferably a CD79b x CD20 x CD3-trispecific antibody as described herein, or a CD79b x CD20 x CD3-trispecific antigen-binding fragment thereof. The instructions may include directions for using the multispecific antibody or antigen-binding fragment thereof in vitro, in vivo or ex vivo.

Typically, the kit will have a compartment containing the multispecific antibody or antigen-binding fragment thereof. The multispecific antibody or antigen-binding fragment thereof may be in a lyophilized form, liquid form, or other form amendable to being included in a kit. The kit may also contain additional elements needed to practice the method described on the instructions in the kit, such a sterilized solution for reconstituting a lyophilized powder, additional agents for combining with the multispecific antibody or antigen-binding fragment thereof prior to administering to a patient, and tools that aid in administering the multispecific antibody or antigen-binding fragment thereof to a patient.

### Diagnostic Uses

The multispecific antibodies and fragments described herein may also be used for diagnostic purposes. Thus, also provided are diagnostic compositions comprising a multispecific antibody or fragments as defined herein, and to its use. In preferred embodiments, the multispecific antibody is a CD79b x CD20 x CD3-multispecific antibody as described herein, or a multispecific antigen-binding fragment thereof, and more preferably a CD79b x CD20 x CD3-trispecific antibody as described herein, or a CD79b x CD20 x CD3-trispecific antigen-binding fragment thereof. In one embodiment, the present invention provides a kit for diagnosis of cancer comprising a container comprising a CD79b x CD20 x CD3 trispecific antibody, and one or more reagents for detecting binding of the antibody to CD79b and/or CD20. Reagents may include, for example, fluorescent tags, enzymatic tags, or other detectable tags. The reagents may also include secondary or tertiary antibodies or reagents for enzymatic reactions, wherein the enzymatic reactions produce a product that may be visualized. For example, the multispecific antibodies described herein, or antigen-binding fragments thereof, may be labeled with a radiolabel, a fluorescent label, an epitope tag, biotin, a chromophore label, an ECL label, an enzyme, ruthenium, ¹¹¹In-DOTA, ¹¹¹In-diethylenetriaminepentaacetic acid (DTPA), horseradish peroxidase, alkaline phosphatase and beta-galactosidase, or poly-histidine or similar such labels known in the art.

### CD79b-Specific Antibodies

Described herein are isolated antibodies and antigen-binding fragments specific for CD79b. In some embodiments, the CD79b-specific antibodies and antigen-binding fragments bind human CD79b. The general structure of an CD79b-specific antibody molecule may comprise an antigen binding domain, which includes heavy and light chains, and the Fc domain, which serves a variety of functions, including complement fixation and binding antibody receptors.

In some embodiments are provided a CD79b-specific antibody, or an antigen-binding fragment thereof, comprising a heavy chain comprising a CDR1, a CDR2, and a CDR3 of any one of the antibodies described in Table 1a. In some embodiments are provided a CD79b-specific antibody, or an antigen-binding fragment thereof, comprising a heavy chain comprising a CDR1, a CDR2, and a CDR3 of any one of the antibodies described in Table 1a and a light chain comprising a CDR1, a CDR2, and a CDR3 of any one of the antibodies described in Table 1a.

The heavy chain variable domain and light chain variable domain of antibodies discussed in this section are suitable for inclusion in multispecific (e.g., bispecific or trispecific) constructs in which one arm is an anti-CD79b arm. Exemplary trispecific constructs comprising the CD79b-specific antibody, or an antigen-binding fragment thereof, discussed in this section are provided herein.

In some embodiments, the CD79b-specific antibodies and antigen-binding fragments bind human CD79b and cynomolgus monkey CD79b. In some embodiments, the CD79b-specific antibodies and antigen-binding fragments bind human CD79b but not to cynomolgus monkey CD79b. In some embodiments, the CD79b-specific antibodies and antigen-binding fragments bind to an epitope including one or more residues from the CD79b extracellular domain (ECD). In some embodiments, the CD79b-binding arm binds to one or more residues of a polypeptide having the amino acid sequence of SEQ ID NO: 252. In some embodiments, the CD79b-binding arm binds bind to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b. Such CD79b-binding arms may bind to CD79b with an affinity of 5×10⁻⁷M or less, such as 1×10⁻⁷M or less, 5×10⁻⁸M or less, 1×10⁻⁸M or less, 5×10⁻⁹M or less, 1×10⁻⁹M, or 5×10⁻¹⁰ M or less. In one embodiment, the CD79b-binding arm binds to the CD79b with an affinity of about 1×10⁻¹¹M to 1×10⁻⁹M. In one embodiment, the CD79b-binding arm binds to the CD79b with an affinity of about 1×10⁻¹¹M, about 2×10⁻¹¹M, about 3×10⁻¹¹M, about 4×10⁻¹¹M, about 5×10⁻¹¹M, about 6×10⁻¹¹M, about 7×10⁻¹¹M, about 8×10⁻¹¹M, about 9×10⁻¹¹M, 1×10⁻¹⁰M, about 2×10⁻¹⁰M, about 3×10⁻¹⁰M, about 4×10⁻¹⁰M, about 5×10⁻¹⁰M, about 6×10⁻¹⁰M, about 7×10⁻¹⁰M, about 8×10⁻¹⁰M, about 9×10⁻¹⁰ M or about 1×10⁻⁹M.

The IgG class is divided in four isotypes: IgG1, IgG2, IgG3 and IgG4 in humans. They share more than 95% homology in the amino acid sequences of the Fc regions but show major differences in the amino acid composition and structure of the hinge region. The Fc region mediates effector functions, such as antibody-dependent cellular cytotoxicity (ADCC) and complement-dependent cytotoxicity (CDC). In ADCC, the Fc region of an antibody binds to Fc receptors (FcγRs) on the surface of immune effector cells such as natural killers and macrophages, leading to the phagocytosis or lysis of the targeted cells. In CDC, the antibodies kill the targeted cells by triggering the complement cascade at the cell surface. The antibodies described herein include antibodies with the described features of the variable domains in combination with any of the IgG isotypes, including modified versions in which the Fc sequence has been modified to effect different effector functions.

For many applications of therapeutic antibodies, Fc-mediated effector functions are not part of the mechanism of action. These Fc-mediated effector functions can be detrimental and potentially pose a safety risk by causing off-mechanism toxicity. Modifying effector functions can be achieved by engineering the Fc regions to reduce their binding to FcγRs or the complement factors. The binding of IgG to the activating (FcγRI, FcγRIIa, FcγRIIIa and FcγRIIIb) and inhibitory (FcγRIIb) FcγRs or the first component of complement (C1q) depends on residues located in the hinge region and the CH2 domain. Mutations have been introduced in IgG1, IgG2 and IgG4 to reduce or silence Fc functionalities. The antibodies described herein may include these modifications.

In one embodiment, the antibody comprises an Fc region with one or more of the following properties: (a) reduced effector function when compared to the parent Fc; (b) reduced affinity to FcγRI, FcγRIIa, FcγRIIb, FcγRIIIb and/or FcγRIIIa, (c) reduced affinity to FcγRI (d) reduced affinity to FcγRIIa (e) reduced affinity to FcγRIIb, (f) reduced affinity to FcγRIIIb or (g) reduced affinity to FcγRIIIa.

In some embodiments, the antibodies or antigen-binding fragments are IgG, or derivatives thereof, e.g., IgG1, IgG2, IgG3, and IgG4 isotypes. In some embodiments wherein the antibody has an IgG1 isotype, the antibody contains L234A, L235A, D265S and/or K409R substitutions in its Fc region. In some embodiments wherein the antibody has an IgG4 isotype, the antibody contains K409R, S228P, L234A, and L235A substitutions in its Fc region. The antibodies described herein may include these modifications.

In some embodiments the described antibodies may be capable of inhibiting APRIL binding with a IC₅₀ of low nanomolar as measured by ELISA.

In some embodiments the described antibodies bind to CD79b-positive multiple myeloma cell lines.

In addition to the described CD79b-specific antibodies and antigen-binding fragments, also provided are polynucleotide sequences capable of encoding the described antibodies and antigen-binding fragments. Vectors comprising the described polynucleotides are also provided, as are cells expressing the CD79b-specific antibodies or antigen-binding fragments provided herein. Also described are cells capable of expressing the disclosed vectors. These cells may be mammalian cells (such as 293F cells, CHO cells), insect cells (such as Sf7 cells), yeast cells, plant cells, or bacteria cells (such as E. coli). The described antibodies may also be produced by hybridoma cells.

The described CD79b-specific antibodies or antigen-binding fragments include all isotypes, IgA, IgD, IgE, IgG and IgM, and synthetic multimers of the four-chain immunoglobulin structure. The described antibodies or antigen-binding fragments also include the IgY isotype generally found in hen or turkey serum and hen or turkey egg yolk.

The CD79b-specific antibodies and antigen-binding fragments may be derived from any species by recombinant means. For example, the antibodies or antigen-binding fragments may be mouse, rat, goat, horse, swine, bovine, chicken, rabbit, camelid, donkey, human, or chimeric versions thereof. For use in administration to humans, non-human derived antibodies or antigen-binding fragments may be genetically or structurally altered to be less antigenic upon administration to a human patient.

In some embodiments, the antibodies or antigen-binding fragments are chimeric. As used herein, the term “chimeric” refers to an antibody, or antigen-binding fragment thereof, having at least some portion of at least one variable domain derived from the antibody amino acid sequence of a non-human mammal, a rodent, or a reptile, while the remaining portions of the antibody, or antigen-binding fragment thereof, are derived from a human.

In some embodiments, the antibodies are humanized antibodies. Humanized antibodies may be chimeric immunoglobulins, immunoglobulin chains or fragments thereof (such as Fv, Fab, Fab′, F(ab′)2 or other antigen-binding subsequences of antibodies) that contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin sequence. The humanized antibody may include at least a portion of an immunoglobulin constant region (Fc), typically that of a human immunoglobulin.

The antibodies or antigen-binding fragments described herein can occur in a variety of forms, but will include one or more of the antibody CDRs shown in Table 1a.

Described herein are recombinant antibodies and antigen-binding fragments that bind to CD79b. In some embodiments, the CD79b-specific antibodies or antigen-binding fragments are human IgG, or derivatives thereof. While the CD79b-specific antibodies or antigen-binding fragments exemplified herein are human, the antibodies or antigen-binding fragments exemplified may be chimerized.

In some embodiments, the antibodies or antigen-binding fragments are IgG, or derivatives thereof, e.g., IgG1, IgG2, IgG3, and IgG4 isotypes. In some embodiments wherein the antibody is of IgG1 isotype, the antibody comprises an IgG1 Fc region (SEQ ID NO: 249).

SEQ ID NO: 249
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments wherein the antibody is of IgG1 isotype, the antibody comprises L234A, L235A, and D265S substitutions (underlined) in its Fc region (SEQ ID NO: 250).

SEQ ID NO: 250
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
SVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

In some embodiments wherein the antibody is of IgG4 isotype, the antibody comprises S228P, L234A, and L235A substitutions (underlined) in its Fc region (SEQ ID NO: 251).

SEQ ID NO: 251
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

The CD79b specific antibodies defined by CDR and/or variable domain sequence discussed in the above paragraphs may include these IgG Fc regions.

Also disclosed are isolated synthetic polynucleotides that encode the antibodies or antigen-binding fragments that bind to CD79b. The isolated polynucleotides capable of encoding the variable domain segments provided herein may be included on the same, or different, vectors to produce antibodies or antigen-binding fragments.

Polynucleotides encoding recombinant antigen-binding proteins also are within the scope of the disclosure. In some embodiments, the polynucleotides described (and the peptides they encode) include a leader sequence. Any leader sequence known in the art may be employed. The leader sequence may include, but is not limited to, a restriction site or a translation start site.

The CD79b-specific antibodies or antigen-binding fragments described herein include variants having single or multiple amino acid substitutions, deletions, or additions that retain the biological properties (e.g., binding affinity or immune effector activity) of the described CD79b-specific antibodies or antigen-binding fragments. In the context of the present invention the following notations are, unless otherwise indicated, used to describe a mutation; i) substitution of an amino acid in a given position is written as e.g. K409R which means a substitution of a Lysine in position 409 with an Arginine; and ii) for specific variants the specific three or one letter codes are used, including the codes Xaa and X to indicate any amino acid residue. Thus, the substitution of Arginine for Lysine in position 409 is designated as: K409R, or the substitution of any amino acid residue for Lysine in position 409 is designated as K409X. In case of deletion of Lysine in position 409 it is indicated by K409*. The skilled person may produce variants having single or multiple amino acid substitutions, deletions, or additions.

These variants may include: (a) variants in which one or more amino acid residues are substituted with conservative or nonconservative amino acids, (b) variants in which one or more amino acids are added to or deleted from the polypeptide, (c) variants in which one or more amino acids include a substituent group, and (d) variants in which the polypeptide is fused with another peptide or polypeptide such as a fusion partner, a protein tag or other chemical moiety, that may confer useful properties to the polypeptide, such as, for example, an epitope for an antibody, a polyhistidine sequence, a biotin moiety and the like. Antibodies or antigen-binding fragments described herein may include variants in which amino acid residues from one species are substituted for the corresponding residue in another species, either at the conserved or nonconserved positions. In other embodiments, amino acid residues at nonconserved positions are substituted with conservative or nonconservative residues. The techniques for obtaining these variants, including genetic (deletions, mutations, etc.), chemical, and enzymatic techniques, are known to persons having ordinary skill in the art.

The CD79b-specific antibodies or antigen-binding fragments described herein may embody several antibody isotypes, such as IgM, IgD, IgG, IgA and IgE. In some embodiments the antibody isotype is IgG1, IgG2, IgG3, or IgG4 isotype, preferably IgG1 or IgG4 isotype. Antibody or antigen-binding fragment thereof specificity is largely determined by the amino acid sequence, and arrangement, of the CDRs. Therefore, the CDRs of one isotype may be transferred to another isotype without altering antigen specificity. Alternatively, techniques have been established to cause hybridomas to switch from producing one antibody isotype to another (isotype switching) without altering antigen specificity. Accordingly, such antibody isotypes are within the scope of the described antibodies or antigen-binding fragments.

Also provided are vectors comprising the polynucleotides described herein. The vectors can be expression vectors. Recombinant expression vectors containing a sequence encoding a polypeptide of interest are thus contemplated as within the scope of this disclosure. The expression vector may contain one or more additional sequences such as but not limited to regulatory sequences (e.g., promoter, enhancer), a selection marker, and a polyadenylation signal. Vectors for transforming a wide variety of host cells are well known and include, but are not limited to, plasmids, phagemids, cosmids, baculoviruses, bacmids, bacterial artificial chromosomes (BACs), yeast artificial chromosomes (YACs), as well as other bacterial, yeast and viral vectors.

Recombinant expression vectors within the scope of the description include synthetic, genomic, or cDNA-derived nucleic acid fragments that encode at least one recombinant protein which may be operably linked to suitable regulatory elements. Such regulatory elements may include a transcriptional promoter, sequences encoding suitable mRNA ribosomal binding sites, and sequences that control the termination of transcription and translation. Expression vectors, especially mammalian expression vectors, may also include one or more nontranscribed elements such as an origin of replication, a suitable promoter and enhancer linked to the gene to be expressed, other 5′ or 3′ flanking nontranscribed sequences, 5′ or 3′ nontranslated sequences (such as necessary ribosome binding sites), a polyadenylation site, splice donor and acceptor sites, or transcriptional termination sequences. An origin of replication that confers the ability to replicate in a host may also be incorporated.

The transcriptional and translational control sequences in expression vectors to be used in transforming vertebrate cells may be provided by viral sources. Exemplary vectors may be constructed as described by Okayama and Berg, 3 Mol. Cell. Biol. 280 (1983).

In some embodiments, the antibody- or antigen-binding fragment-coding sequence is placed under control of a powerful constitutive promoter, such as the promoters for the following genes: hypoxanthine phosphoribosyl transferase (HPRT), adenosine deaminase, pyruvate kinase, beta-actin, human myosin, human hemoglobin, human muscle creatine, and others. In addition, many viral promoters function constitutively in eukaryotic cells and are suitable for use with the described embodiments. Such viral promoters include without limitation, Cytomegalovirus (CMV) immediate early promoter, the early and late promoters of SV40, the Mouse Mammary Tumor Virus (MMTV) promoter, the long terminal repeats (LTRs) of Maloney leukemia virus, Human Immunodeficiency Virus (HIV), Epstein Barr Virus (EBV), Rous Sarcoma Virus (RSV), and other retroviruses, and the thymidine kinase promoter of Herpes Simplex Virus. In one embodiment, the CD79b-specific antibody or antigen-binding fragment thereof coding sequence is placed under control of an inducible promoter such as the metallothionein promoter, tetracycline-inducible promoter, doxycycline-inducible promoter, promoters that contain one or more interferon-stimulated response elements (ISRE) such as protein kinase R 2′,5′-oligoadenylate synthetases, Mx genes, ADAR1, and the like.

Vectors described herein may contain one or more Internal Ribosome Entry Site(s) (IRES). Inclusion of an IRES sequence into fusion vectors may be beneficial for enhancing expression of some proteins. In some embodiments the vector system will include one or more polyadenylation sites (e.g., SV40), which may be upstream or downstream of any of the aforementioned nucleic acid sequences. Vector components may be contiguously linked, or arranged in a manner that provides optimal spacing for expressing the gene products (i.e., by the introduction of “spacer” nucleotides between the ORFs), or positioned in another way. Regulatory elements, such as the IRES motif, may also be arranged to provide optimal spacing for expression.

The vectors may comprise selection markers, which are well known in the art. Selection markers include positive and negative selection markers, for example, antibiotic resistance genes (e.g., neomycin resistance gene, a hygromycin resistance gene, a kanamycin resistance gene, a tetracycline resistance gene, a penicillin resistance gene, a puromycin resistance gene, a blasticidin resistance gene), glutamate synthase genes, HSV-TK, HSV-TK derivatives for ganciclovir selection, or bacterial purine nucleoside phosphorylase gene for 6-methylpurine selection (Gadi et al., 7 Gene Ther. 1738-1743 (2000)). A nucleic acid sequence encoding a selection marker or the cloning site may be upstream or downstream of a nucleic acid sequence encoding a polypeptide of interest or cloning site.

The vectors described herein may be used to transform various cells with the genes encoding the described antibodies or antigen-binding fragments. For example, the vectors may be used to generate CD79b-specific antibody or antigen-binding fragment-producing cells. Thus, another aspect features host cells transformed with vectors comprising a nucleic acid sequence encoding an antibody or antigen-binding fragment thereof that binds CD79b, such as the antibodies or antigen-binding fragments described and exemplified herein.

Numerous techniques are known in the art for the introduction of foreign genes into cells and may be used to construct the recombinant cells for purposes of carrying out the described methods, in accordance with the various embodiments described and exemplified herein. The technique used should provide for the stable transfer of the heterologous gene sequence to the host cell, such that the heterologous gene sequence is heritable and expressible by the cell progeny, and so that the necessary development and physiological functions of the recipient cells are not disrupted. Techniques which may be used include but are not limited to chromosome transfer (e.g., cell fusion, chromosome mediated gene transfer, micro cell mediated gene transfer), physical methods (e.g., transfection, spheroplast fusion, microinjection, electroporation, liposome carrier), viral vector transfer (e.g., recombinant DNA viruses, recombinant RNA viruses) and the like (described in Cline, 29 Pharmac. Ther. 69-92 (1985)). Calcium phosphate precipitation and polyethylene glycol (PEG)-induced fusion of bacterial protoplasts with mammalian cells may also be used to transform cells.

Cells suitable for use in the expression of the CD79b-specific antibodies or antigen-binding fragments described herein are preferably eukaryotic cells, more preferably cells of plant, rodent, or human origin, for example but not limited to NSO, CHO, CHOK1, perC.6, Tk-ts13, BHK, HEK293 cells, COS-7, T98G, CV-1/EBNA, L cells, C127, 3T3, HeLa, NS1, Sp2/0 myeloma cells, and BHK cell lines, among others. In addition, expression of antibodies may be accomplished using hybridoma cells. Methods for producing hybridomas are well established in the art.

Cells transformed with expression vectors described herein may be selected or screened for recombinant expression of the antibodies or antigen-binding fragments described herein. Recombinant-positive cells are expanded and screened for subclones exhibiting a desired phenotype, such as high level expression, enhanced growth properties, or the ability to yield proteins with desired biochemical characteristics, for example, due to protein modification or altered post-translational modifications. These phenotypes may be due to inherent properties of a given subclone or to mutation. Mutations may be effected through the use of chemicals, UV-wavelength light, radiation, viruses, insertional mutagens, inhibition of DNA mismatch repair, or a combination of such methods.

### Methods of Using CD79b-Specific Antibodies for Treatment

Provided herein are CD79b-specific antibodies or antigen-binding fragments thereof for use in therapy. In particular, these antibodies or antigen-binding fragments may be useful in treating cancer, such as CD79b-expressing cancer. Accordingly, the invention provides a method of treating cancer comprising administering an antibody as described herein, such as CD79b-specific antibodies or antigen-binding fragments. For example, the use may be by interfering with CD79b-receptor interactions or where the antibody is conjugated to a toxin, so targeting the toxin to the CD79b-expressing cancer. In some embodiments CD79b-expressing cancer includes lymphoma, such as diffuse large B-cell lymphoma (DLBCL). The antibodies for use in these methods include those described herein above, for example a CD79b-specific antibody or antigen-binding fragment with the features set out in Table 1a and Table 1b, for example the CDRs or variable domain sequences, and in the further discussion of these antibodies.

In some embodiments described herein, immune effector properties of the CD79b-specific antibodies may be enhanced or silenced through Fc modifications by techniques known to those skilled in the art and described herein. For example, Fc effector functions such as C1q binding, complement dependent cytotoxicity (CDC), antibody-dependent cell-mediated cytotoxicity (ADCC), antibody-dependent cell-mediated phagocytosis (ADCP), down regulation of cell surface receptors (e.g., B cell receptor; BCR), etc. may be provided and/or controlled by modifying residues in the Fc responsible for these activities.

“Antibody-dependent cell-mediated cytotoxicity” or “ADCC” refers to a cell-mediated reaction in which non-specific cytotoxic cells that express Fc receptors (FcRs) (e.g. Natural Killer (NK) cells, neutrophils, and macrophages) recognize bound antibody on a target cell and subsequently cause lysis of the target cell.

The ability of monoclonal antibodies to induce ADCC can be enhanced by engineering their oligosaccharide component. Human IgG1 or IgG3 are N-glycosylated at Asn297 with the majority of the glycans in the well-known biantennary G0, G0F, G1, G1F, G2 or G2F forms. Antibodies produced by non-engineered CHO cells typically have a glycan fucose content of about at least 85%. The removal of the core fucose from the biantennary complex-type oligosaccharides attached to the Fc regions enhances the ADCC of antibodies via improved FcγRIIIa binding without altering antigen binding or CDC activity. Such mAbs can be achieved using different methods reported to lead to the successful expression of relatively high defucosylated antibodies bearing the biantennary complex-type of Fc oligosaccharides such as control of culture osmolality (Konno et al., Cytotechnology 64:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the .alpha. 1,6-fucosyltrasferase (FUT8) gene (Mori et al., Biotechnol Bioeng 88:901-908, 2004), or coexpression of β-1,4-N-acetylglucosaminyltransferase III and golgi α-mannosidase II or a potent alpha-mannosidase I inhibitor, kifunensine (Ferrara et al., J Biol Chem 281:5032-5036, 2006, Ferrara et al., Biotechnol Bioeng 93:851-861, 2006; Xhou et al., Biotechnol Bioeng 99:652-65, 2008).

In some embodiments described herein, ADCC elicited by the CD79b antibodies may also be enhanced by certain substitutions in the antibody Fc. Exemplary substitutions are for example substitutions at amino acid positions 256, 290, 298, 312, 356, 330, 333, 334, 360, 378 or 430 (residue numbering according to the EU index) as described in U.S. Pat. No. 6,737,056.

### Methods of Detecting CD79b

Provided herein are methods for detecting CD79b in a biological sample by contacting the sample with an antibody, or antigen-binding fragment thereof, described herein. As described herein, the sample may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like. In some embodiments the described methods include detecting CD79b in a biological sample by contacting the sample with any of the CD79b-specific antibodies or antigen-binding fragments thereof described herein.

In some embodiments the sample may be contacted with more than one of the CD79b-specific antibodies or antigen-binding fragments described herein. For example, a sample may be contacted with a first CD79b-specific antibody, or antigen-binding fragment thereof, and then contacted with a second CD79b-specific antibody, or antigen-binding fragment thereof, wherein the first antibody or antigen-binding fragment and the second antibody or antigen-binding fragment are not the same antibody or antigen-binding fragment. In some embodiments, the first antibody, or antigen-binding fragment thereof, may be affixed to a surface, such as a multiwell plate, chip, or similar substrate prior to contacting the sample. In other embodiments the first antibody, or antigen-binding fragment thereof, may not be affixed, or attached, to anything at all prior to contacting the sample.

The described CD79b-specific antibodies and antigen-binding fragments may be detectably labeled. In some embodiments labeled antibodies and antigen-binding fragments may facilitate the detection CD79b via the methods described herein. Many such labels are readily known to those skilled in the art. For example, suitable labels include, but should not be considered limited to, radiolabels, fluorescent labels, epitope tags, biotin, chromophore labels, ECL labels, or enzymes. More specifically, the described labels include ruthenium, ¹¹¹In-DOTA, ¹¹¹In-diethylenetriaminepentaacetic acid (DTPA), horseradish peroxidase, alkaline phosphatase and beta-galactosidase, poly-histidine (HIS tag), acridine dyes, cyanine dyes, fluorone dyes, oxazin dyes, phenanthridine dyes, rhodamine dyes, Alexafluor® dyes, and the like.

The described CD79b-specific antibodies and antigen-binding fragments may be used in a variety of assays to detect CD79b in a biological sample. Some suitable assays include, but should not be considered limited to, western blot analysis, radioimmunoassay, surface plasmon resonance, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay.

In some embodiments described herein detection of CD79b-expressing cancer cells in a subject may be used to determine that the subject may be treated with a therapeutic agent directed against CD79b.

CD79b is present at detectable levels in blood and serum samples. Thus, provided herein are methods for detecting CD79b in a sample derived from blood, such as a serum sample, by contacting the sample with an antibody, or antigen-binding fragment thereof, that binds CD79b. The blood sample, or a derivative thereof, may be diluted, fractionated, or otherwise processed to yield a sample upon which the described method may be performed. In some embodiments, CD79b may be detected in a blood sample, or a derivative thereof, by any number of assays known in the art, such as, but not limited to, western blot analysis, radioimmunoassay, surface plasmon resonance, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay.

### Methods for Diagnosing Cancer

Provided herein are methods for diagnosing CD79b-expressing cancer in a subject. In some embodiments CD79b-expressing cancer include lymphomas, such as diffuse large B-cell lymphoma (DLBCL). In some embodiments, as described above, detecting CD79b in a biological sample, such as a blood sample or a serum sample, provides the ability to diagnose cancer in the subject from whom the sample was obtained. Alternatively, in some embodiments other samples such as a histological sample, a fine needle aspirate sample, resected tumor tissue, circulating cells, circulating tumor cells, and the like, may also be used to assess whether the subject from whom the sample was obtained has cancer. In some embodiments, it may already be known that the subject from whom the sample was obtained has cancer, but the type of cancer afflicting the subject may not yet have been diagnosed or a preliminary diagnosis may be unclear, thus detecting CD79b in a biological sample obtained from the subject can allow for, or clarify, diagnosis of the cancer. For example, a subject may be known to have cancer, but it may not be known, or may be unclear, whether the subject's cancer is CD79b-expressing.

In some embodiments the described methods involve assessing whether a subject is afflicted with CD79b-expressing cancer by determining the amount of CD79b that is present in a biological sample derived from the subject; and comparing the observed amount of CD79b with the amount of CD79b in a control, or reference, sample, wherein a difference between the amount of CD79b in the sample derived from the subject and the amount of CD79b in the control, or reference, sample is an indication that the subject is afflicted with a CD79b-expressing cancer. In another embodiment the amount of CD79b observed in a biological sample obtained from a subject may be compared to levels of CD79b known to be associated with certain forms or stages of cancer, to determine the form or stage of the subject's cancer. In some embodiments the amount of CD79b in the sample derived from the subject is assessed by contacting the sample with an antibody, or an antigen-binding fragment thereof, that binds CD79b, such as the CD79b-specific antibodies described herein. The sample assessed for the presence of CD79b may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like. In some embodiments CD79b-expressing cancer includes hematological cancer, such as diffuse large B-cell lymphoma (DLBCL). In some embodiments the subject is a human.

In some embodiments the method of diagnosing a CD79b-expressing cancer will involve: contacting a biological sample of a subject with a CD79b-specific antibody, or an antigen-binding fragment thereof (such as those derivable from the antibodies and fragments provided in Table 1a and Table 1b), quantifying the amount of CD79b present in the sample that is bound by the antibody or antigen-binding fragment thereof, comparing the amount of CD79b present in the sample to a known standard or reference sample; and determining whether the subject's CD79b levels fall within the levels of CD79b associated with cancer. In an additional embodiment, the diagnostic method can be followed with an additional step of administering or prescribing a cancer-specific treatment. In another embodiment, the diagnostic method can be followed with an additional step of transmitting the results of the determination to facilitate treatment of the cancer. In some embodiments the cancer-specific treatment may be directed against CD79b-expressing cancers, such as the CD79b x CD3 multispecific antibodies described herein.

In some embodiments the described methods involve assessing whether a subject is afflicted with CD79b-expressing cancer by determining the amount of CD79b present in a blood or serum sample obtained from the subject; and comparing the observed amount of CD79b with the amount of CD79b in a control, or reference, sample, wherein a difference between the amount of CD79b in the sample derived from the subject and the amount of CD79b in the control, or reference, sample is an indication that the subject is afflicted with a CD79b-expressing cancer.

In some embodiments the control, or reference, sample may be derived from a subject that is not afflicted with CD79b-expressing cancer. In some embodiments the control, or reference, sample may be derived from a subject that is afflicted with CD79b-expressing cancer. In some embodiments where the control, or reference, sample is derived from a subject that is not afflicted with CD79b-expressing cancer, an observed increase in the amount of CD79b present in the test sample, relative to that observed for the control or reference sample, is an indication that the subject being assessed is afflicted with CD79b-expressing cancer. In some embodiments where the control sample is derived from a subject that is not afflicted with CD79b-expressing cancer, an observed decrease or similarity in the amount of CD79b present in the test sample, relative to that observed for the control or reference sample, is an indication that the subject being assessed is not afflicted with CD79b-expressing cancer. In some embodiments where the control or reference sample is derived from a subject that is afflicted with CD79b-expressing cancer, an observed similarity in the amount of CD79b present in the test sample, relative to that observed for the control or reference sample, is an indication that the subject being assessed is afflicted with CD79b-expressing cancer. In some embodiments where the control or reference sample is derived from a subject that is afflicted with CD79b-expressing cancer, an observed decrease in the amount of CD79b present in the test sample, relative to that observed for the control or reference sample, is an indication that the subject being assessed is not afflicted with CD79b-expressing cancer.

In some embodiments the amount of CD79b in the sample derived from the subject is assessed by contacting the sample with an antibody, or an antigen-binding fragment thereof, that binds CD79b, such as the antibodies described herein. The sample assessed for the presence of CD79b may be derived from a blood sample, a serum sample, circulating cells, circulating tumor cells, cells that are not tissue associated (i.e., free cells), tissues (e.g., surgically resected tumor tissue, biopsies, including fine needle aspiration), histological preparations, and the like.

In various aspects, the amount of CD79b is determined by contacting the sample with an antibody, or antigen-binding fragment thereof, that binds CD79b. In some embodiments, the sample may be contacted by more than one type of antibody, or antigen-binding fragment thereof, that binds CD79b. In some embodiments, the sample may be contacted by a first antibody, or antigen-binding fragment thereof, that binds CD79b and then contacted by a second antibody, or antigen-binding fragment thereof, that binds CD79b. CD79b-specific antibodies or antigen-binding fragments such as those described herein may be used in this capacity.

Various combinations of the CD79b-specific antibodies and antigen-binding fragments can be used to provide a “first” and “second” antibody or antigen-binding fragment to carry out the described diagnostic methods. In some embodiments CD79b-expressing cancer includes lymphomas, such as diffuse large B-cell lymphoma (DLBCL).

In certain embodiments, the amount of CD79b is determined by western blot analysis, radioimmunoassay, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay.

In various embodiments of the described diagnostic methods a control or reference sample is used. This sample may be a positive or negative assay control that ensures the assay used is working properly; for example, an assay control of this nature might be commonly used for immunohistochemistry assays. Alternatively, the sample may be a standardized reference for the amount of CD79b in a biological sample from a healthy subject. In some embodiments, the observed CD79b levels of the tested subject may be compared with CD79b levels observed in samples from subjects known to have CD79b-expressing cancer. In some embodiments, the control subject may be afflicted with a particular cancer of interest. In some embodiments, the control subject is known to have early stage cancer, which may or may not be CD79b-expressing cancer. In some embodiments, the control subject is known to have intermediate stage cancer, which may or may not be CD79b-expressing cancer. In some embodiments, the control subject is known to have late stage, which may or may not be CD79b-expressing cancer. In some embodiments, the methods for diagnosing cancer or a disorder are in vitro methods.

### Methods for Monitoring Cancer

Provided herein are methods for monitoring CD79b-expressing cancer in a subject. In some embodiments CD79b-expressing cancer includes lymphomas, such as diffuse large B-cell lymphoma (DLBCL). In some embodiments the described methods involve assessing whether CD79b-expressing cancer is progressing, regressing, or remaining stable by determining the amount of CD79b that is present in a test sample derived from the subject; and comparing the observed amount of CD79b with the amount of CD79b in a biological sample obtained, in a similar manner, from the subject at an earlier point in time, wherein a difference between the amount of CD79b in the test sample and the earlier sample provides an indication of whether the cancer is progressing, regressing, or remaining stable. In this regard, a test sample with an increased amount of CD79b, relative to the amount observed for the earlier sample, may indicate progression of a CD79b-expressing cancer. Conversely, a test sample with a decreased amount of CD79b, relative to the amount observed for the earlier sample, may indicate regression of a CD79b-expressing cancer.

Accordingly, a test sample with an insignificant difference in the amount of CD79b, relative to the amount observed for the earlier sample, may indicate a state of stable disease for a CD79b-expressing cancer. In some embodiments the amount of CD79b in a biological sample derived from the subject is assessed by contacting the sample with an antibody, or an antibody fragment thereof, that binds CD79b, such as the antibodies described herein. The sample assessed for the presence of CD79b may be derived from urine, blood, serum, plasma, saliva, ascites, circulating cells, circulating tumor cells, cells that are not tissue associated (i.e., free cells), tissues (e.g., surgically resected tumor tissue, biopsies, including fine needle aspiration), histological preparations, and the like. In some embodiments the subject is a human.

In some embodiments the methods of monitoring a CD79b-expressing cancer will involve: contacting a biological sample of a subject with a CD79b-specific antibody, or antigen-binding fragment thereof (such as those derivable from the antibodies and fragments provided in Table 1a and Table 1b), quantifying the amount of CD79b present in the sample, comparing the amount of CD79b present in the sample to the amount of CD79b determined to be in a biological sample obtained, in a similar manner, from the same subject at an earlier point in time; and determining whether the subject's CD79b level has changed over time. A test sample with an increased amount of CD79b, relative to the amount observed for the earlier sample, may indicate progression of cancer. Conversely, a test sample with a decreased amount of CD79b, relative to the amount observed for the earlier sample, may indicate regression of a CD79b-expressing cancer. Accordingly, a test sample with an insignificant difference in the amount of CD79b, relative to the amount observed for the earlier sample, may indicate a state of stable disease for a CD79b-expressing cancer. In some embodiments, the CD79b levels of the sample may be compared to a known standard or a reference sample, alone or in addition to the CD79b levels observed for a sample assessed at an earlier point in time. In an additional embodiment, the diagnostic method can be followed with an additional step of administering a cancer-specific treatment. In some embodiments the cancer-specific treatment may be directed against CD79b-expressing cancers, such as the CD79b x CD3 multispecific antibodies described herein.

In various aspects, the amount of CD79b is determined by contacting the sample with an antibody, or antigen-binding fragment thereof, that binds CD79b. In some embodiments, the sample may be contacted by more than one type of antibody, or antigen-binding fragment thereof, that binds CD79b. In some embodiments, the sample may be contacted by a first antibody, or antigen-binding fragment thereof, that binds CD79b and then contacted by a second antibody, or antigen-binding fragment thereof, that binds CD79b. Antibodies such as those described herein may be used in this capacity.

Various combinations of the antibodies and antigen-binding fragments described in Table 1a and Table 1b can be used to provide a “first” and “second” antibody or antigen-binding fragment to carry out the described monitoring methods. In some embodiments CD79b-expressing cancer includes a hematological cancer, such as acute myeloid leukemia (AML).

In certain embodiments, the amount of CD79b is determined by western blot analysis, radioimmunoassay, immunofluorimetry, immunoprecipitation, equilibrium dialysis, immunodiffusion, electrochemiluminescence (ECL) immunoassay, immunohistochemistry, fluorescence-activated cell sorting (FACS) or ELISA assay. In some embodiments, the monitoring methods are in vitro methods.

### Kits for Detecting CD79b

Provided herein are kits for detecting CD79b in a biological sample. These kits include one or more of the CD79b-specific antibodies described herein, or an antigen-binding fragment thereof, and instructions for use of the kit.

The provided CD79b-specific antibody, or antigen-binding fragment, may be in solution; lyophilized; affixed to a substrate, carrier, or plate; or detectably labeled.

The described kits may also include additional components useful for performing the methods described herein. By way of example, the kits may comprise means for obtaining a sample from a subject, a control or reference sample, e.g., a sample from a subject having slowly progressing cancer and/or a subject not having cancer, one or more sample compartments, and/or instructional material which describes performance of a method of the invention and tissue specific controls or standards.

The means for determining the level of CD79b can further include, for example, buffers or other reagents for use in an assay for determining the level of CD79b. The instructions can be, for example, printed instructions for performing the assay and/or instructions for evaluating the level of expression of CD79b.

The described kits may also include means for isolating a sample from a subject. These means can comprise one or more items of equipment or reagents that can be used to obtain a fluid or tissue from a subject. The means for obtaining a sample from a subject may also comprise means for isolating blood components, such as serum, from a blood sample. Preferably, the kit is designed for use with a human subject.

### EMBODIMENTS

The disclosure provided herein also provides the following non-limiting embodiments.
1. A trispecific antibody, or a trispecific binding fragment thereof, comprising:
(a) a first antigen-binding arm comprising a first heavy chain variable domain (VH1) and a first light chain variable domain (VL1);
(b) a second antigen-binding arm comprising a second heavy chain variable domain (VH2) and a second light chain variable domain (VL2);
(c) a third antigen-binding arm comprising a third heavy chain variable domain (VH3) and a third light chain variable domain (VL3),
wherein the first antigen-binding arm binds to an epitope on cluster of differentiation 79B protein (CD79b), the second antigen-binding arm binds to an epitope on cluster of differentiation 3 (CD3), and the third antigen-binding arm binds to an epitope on cluster of differentiation 20 (CD20).
2. The trispecific antibody or trispecific binding fragment of embodiment 1, wherein the VH1 and VL1 of first antigen-binding arm are present in a diabody, a Fab, Fab′, a F(ab′)2, a Fv, a scFv, a Fd, a disulfide stabilized Fv fragment (dsFv), or a disulfide stabilized diabody (ds diabody), optionally a Fab.
3. The trispecific antibody or trispecific binding fragment of embodiment 1 or 2, wherein the VH2 and VL2 of the second antigen-binding arm are present in a diabody, a Fab, Fab′, a F(ab′)2, a Fv, a scFv, a Fv, a Fd, a disulfide stabilized Fv fragment (dsFv), or a disulfide stabilized diabody (ds diabody), optionally a scFv.
4. The trispecific antibody or trispecific binding fragment of any one of embodiments 1-3, wherein the VH3 and VL3 of the third antigen-binding arm are present in an antibody fragment, a diabody, a Fab, Fab′, a F(ab′)2, a Fv, a scFv, a Fd, a disulfide stabilized Fv fragment (dsFv), or a disulfide stabilized diabody (ds diabody), optionally a scFv.
5. The trispecific antibody or trispecific binding fragment of any one of embodiments 1-4, wherein the first antigen-binding arm that binds CD79b comprises:
   a) a heavy chain complementarity determining region (HCDR) 1, a HCDR2 and a HCDR3 of the heavy chain variable domain (VH1) of SEQ ID NO: 35 and a light chain complementarity determining region (LCDR) 1, a LCDR2 and a LCDR3 of the light chain variable domain (VL1) of SEQ ID NO: 37;
   b) the HCDR1, the HCDR2 and the HCDR3 of the VH1 of SEQ ID NO: 39 and the LCDR1, the LCDR2 and the LCDR3 of the VL1 of SEQ ID NO: 41;
   c) the HCDR1, the HCDR2 and the HCDR3 of the VH1 of SEQ ID NO: 43 and the LCDR1, the LCDR2 and the LCDR3 of the VL1 of SEQ ID NO: 41;
   d) the HCDR1, the HCDR2 and the HCDR3 of the VH1 of SEQ ID NO: 45 and the LCDR1, the LCDR2 and the LCDR3 of the VL1 of SEQ ID NO: 47;
   e) the HCDR1, the HCDR2 and the HCDR3 of the VH1 of SEQ ID NO: 49 and the LCDR1, the LCDR2 and the LCDR3 of the VL1 of SEQ ID NO: 51;
   f) the HCDR1, the HCDR2 and the HCDR3 of the VH1 of SEQ ID NO: 39 and the LCDR1, the LCDR2 and the LCDR3 of the VL1 of SEQ ID NO: 53;
   g) the HCDR1, the HCDR2 and the HCDR3 of the VH1 of SEQ ID NO: 55 and the LCDR1, the LCDR2 and the LCDR3 of the VL1 of SEQ ID NO: 57;
   h) the HCDR1, the HCDR2 and the HCDR3 of the VH1 of SEQ ID NO: 59 and the LCDR1, the LCDR2 and the LCDR3 of the VL1 of SEQ ID NO: 61;
   i) the HCDR1, the HCDR2 and the HCDR3 of the VH1 of SEQ ID NO: 63 and the LCDR1, the LCDR2 and the LCDR3 of the VL1 of SEQ ID NO: 65;
   j) the HCDR1, the HCDR2 and the HCDR3 of the VH1 of SEQ ID NO: 67 and the LCDR1, the LCDR2 and the LCDR3 of the VL1 of SEQ ID NO: 69; or
   k) the HCDR1, the HCDR2 and the HCDR3 of the VH1 of SEQ ID NO: 71 and the LCDR1, the LCDR2 and the LCDR3 of the VL1 of SEQ ID NO: 73.
6. The trispecific antibody or trispecific binding fragment of any one of embodiments 1-5, wherein the first antigen-binding arm that binds CD79b comprises the HCDR1, the HCDR2, the HCDR3, the LCDR1, the LCDR2, and the LCDR3 of:
a) SEQ ID NOs: 1, 2, 3, 4, 5 and 6, respectively;
b) SEQ ID NOs: 13, 8, 9, 10, 11 and 12, respectively;
c) SEQ ID NOs: 7, 8, 9, 10, 11 and 12, respectively;
d) SEQ ID NOs: 14, 15, 16, 17, 5 and 6, respectively;
e) SEQ ID NOs: 18, 8, 19, 20, 21 and 12, respectively;
f) SEQ ID NOs: 22, 23, 24, 25, 5 and 6, respectively;
g) SEQ ID NOs: 22, 26, 27, 28, 5 and 29, respectively; or
h) SEQ ID NOs: 30, 31, 32, 33, 5 and 6, respectively.
7. The trispecific antibody or trispecific binding fragment of any one of embodiments 1-6, wherein the first antigen-binding arm that binds CD79b comprises:
a) the VH1 of SEQ ID NO: 35 and the VL1 of SEQ ID NO: 37;
b) the VH1 of SEQ ID NO: 39 and the VL1 of SEQ ID NO: 41;
c) the VH1 of SEQ ID NO: 43 and the VL1 of SEQ ID NO: 41;
d) the VH1 of SEQ ID NO: 45 and the VL1 of SEQ ID NO: 47;
e) the VH1 of SEQ ID NO: 49 and the VL1 of SEQ ID NO: 51;
f) the VH1 of SEQ ID NO: 39 and the VL1 of SEQ ID NO: 53;
g) the VH1 of SEQ ID NO: 55 and the VL1 of SEQ ID NO: 57;
h) the VH1 of SEQ ID NO: 59 and the VL1 of SEQ ID NO: 61;
i) the VH1 of SEQ ID NO: 63 and the VL1 of SEQ ID NO: 65;
j) the VH1 of SEQ ID NO: 67 and the VL1 of SEQ ID NO: 69; or
i) the VH1 of SEQ ID NO: 71 and the VL1 of SEQ ID NO: 73.
8. The trispecific antibody or trispecific binding fragment of any one of embodiments 1-7, wherein the second antigen-binding arm that binds CD3 comprises:
   a) the HCDR1, the HCDR2 and the HCDR3 of the VH2 of SEQ ID NO: 97 and the LCDR1, the LCDR2 and the LCDR3 of the VL2 of SEQ ID NO: 99;
   b) the HCDR1, the HCDR2 and the HCDR3 of the VH2 of SEQ ID NO: 101 and the LCDR1, the LCDR2 and the LCDR3 of the VL2 of SEQ ID NO: 99;
   c) the HCDR1, the HCDR2 and the HCDR3 of the VH2 of SEQ ID NO: 103 and the LCDR1, the LCDR2 and the LCDR3 of the VL2 of SEQ ID NO: 99;
   d) the HCDR1, the HCDR2 and the HCDR3 of the VH2 of SEQ ID NO: 105 and the LCDR1, the LCDR2 and the LCDR3 of the VL2 of SEQ ID NO: 99; or
   e) the HCDR1, the HCDR2 and the HCDR3 of the VH2 of SEQ ID NO: 107 and the LCDR1, the LCDR2 and the LCDR3 of the VL2 of SEQ ID NO: 109.
9. The trispecific antibody or trispecific binding fragment of any one of embodiments 1-8, wherein the second antigen-binding arm that binds CD3 comprises the HCDR1, the HCDR2, the HCDR3, the LCDR1, the LCDR2 and the LCDR3 of:
a) SEQ ID NOs: 76, 77, 78, 79, 80 and 81, respectively;
b) SEQ ID NOs: 76, 77, 75, 79, 80 and 81, respectively;
c) SEQ ID NOs: 76, 77, 82, 79, 80 and 81, respectively; or
d) SEQ ID NOs: 83, 84, 85, 86, 87 and 88, respectively.
10. The trispecific antibody or trispecific binding fragment of any one of embodiments 1-9, wherein the second antigen-binding arm that binds CD3 comprises:
a) the VH2 of SEQ ID NO: 97 and the VL2 of SEQ ID NO: 99;
b) the VH2 of SEQ ID NO: 101 and the VL2 of SEQ ID NO: 99;
c) the VH2 of SEQ ID NO: 103 and the VL2 of SEQ ID NO: 99;
d) the VH2 of SEQ ID NO: 105 and the VL2 of SEQ ID NO: 99; or
e) the VH2 of SEQ ID NO: 107 and the VL2 of SEQ ID NO: 109.
11. The trispecific antibody or trispecific binding fragment of any one of embodiments 1-10, wherein the third antigen-binding arm that binds CD20 comprises:
   a) the HCDR1, the HCDR2 and the HCDR3 of the VH3 of SEQ ID NO: 126 and the LCDR1, the LCDR2 and the LCDR3 of the VL3 of SEQ ID NO: 128;
   b) the HCDR1, the HCDR2 and the HCDR3 of the VH3 of SEQ ID NO: 130 and the LCDR1, the LCDR2 and the LCDR3 of the VL3 of SEQ ID NO: 132;
   c) the HCDR1, the HCDR2 and the HCDR3 of the VH3 of SEQ ID NO: 134 and the LCDR1, the LCDR2 and the LCDR3 of the VL3 of SEQ ID NO: 136; or
   d) the HCDR1, the HCDR2 and the HCDR3 of the VH3 of SEQ ID NO: 138 and the LCDR1, the LCDR2 and the LCDR3 of the VL3 of SEQ ID NO: 140.
12. The trispecific antibody or trispecific binding fragment of any one of embodiments 1-11, wherein the third antigen-binding arm that binds CD20 comprises the HCDR1, the HCDR2, the HCDR3, the LCDR1, the LCDR2 and the LCDR3 of:
   a) SEQ ID NOs: 115, 116, 117, 118, 119 and 120, respectively;
   b) SEQ ID NOs: 121, 122, 123, 124, 119 and 125, respectively;
   c) SEQ ID NOs: 115, 116, 95, 96, 119 and 125, respectively; or
   d) SEQ ID NOs: 121, 116, 123, 124, 119 and 125, respectively.
13. The trispecific antibody or trispecific binding fragment of any one of embodiments 1-12, wherein the third antigen-binding arm that binds CD20 comprises:
a) the VH3 of SEQ ID NO: 126 and the VL3 of SEQ ID NO: 128;
b) the VH3 of SEQ ID NO: 130 and the VL3 of SEQ ID NO: 132;
c) the VH3 of SEQ ID NO: 134 and the VL3 of SEQ ID NO: 136; or
d) the VH3 of SEQ ID NO: 138 and the VL3 of SEQ ID NO: 140.
14. The trispecific antibody or trispecific binding fragment of any one of embodiments 1-4, wherein the first antigen-binding arm that binds CD79b comprises the HCDR1, the HCDR2 and the HCDR3 of the VH1 of SEQ ID NO: 35 and the LCDR1, the LCDR2 and the LCDR3 of the VL1 of SEQ ID NO: 37;
the second antigen-binding arm that binds CD3 comprises the HCDR1, the HCDR2 and the HCDR3 of the VH2 of SEQ ID NO: 107 and the LCDR1, the LCDR2 and the LCDR3 of the VL2 of SEQ ID NO: 109; and
the third antigen-binding arm that binds CD20 comprises the HCDR1, the HCDR2 and the HCDR3 of the VH3 of SEQ ID NO: 130 and the LCDR1, the LCDR2 and the LCDR3 of the VL3 of SEQ ID NO: 132.
15. The trispecific antibody or trispecific binding fragment of any one of embodiments 1-4 and 14, wherein the first antigen-binding arm that binds CD79b comprises the HCDR1, the HCDR2, the HCDR3, the LCDR1, the LCDR2 and the LCDR3 of SEQ ID NOs: 1, 2, 3, 4, 5 and 6, respectively;
the second antigen-binding arm that binds CD3 comprises the HCDR1, the HCDR2, the HCDR3, the LCDR1, the LCDR2 and the LCDR3 of SEQ ID NOs: 83, 84, 85, 86, 87 and 88, respectively; and
the third antigen-binding arm that binds CD20 comprises the HCDR1, the HCDR2, the HCDR3, the LCDR1, the LCDR2 and the LCDR3 of SEQ ID NOs: 121, 122, 123, 124, 119 and 125, respectively.
16. The trispecific antibody or trispecific binding fragment of any one of embodiments 1-4, 14 and 15, wherein the first antigen-binding arm that binds CD79b comprises the VH1 of SEQ ID NO: 35 and the VL1 of SEQ ID NO: 37;
the second antigen-binding arm that binds CD3 comprises the VH2 of SEQ ID NO: 107 and the VL2 of SEQ ID NO: 109; and
the third antigen-binding arm that binds CD20 comprises the VH3 of SEQ ID NO: 130 and the VL3 of SEQ ID NO: 132.
17. The trispecific antibody or trispecific binding fragment of any one of embodiments 1-4, wherein the first antigen-binding arm that binds CD79b comprises the HCDR1, the HCDR2 and the HCDR3 of the VH1 of SEQ ID NO: 35 and the LCDR1, the LCDR2 and the LCDR3 of the VL1 of SEQ ID NO: 37;
the second antigen-binding arm that binds CD3 comprises the HCDR1, the HCDR2 and the HCDR3 of the VH2 of SEQ ID NO: 101 and the LCDR1, the LCDR2 and the LCDR3 of the VL2 of SEQ ID NO: 99; and
the third antigen-binding arm that binds CD20 comprises the HCDR1, the HCDR2 and the HCDR3 of the VH3 of SEQ ID NO: 130 and the LCDR1, the LCDR2 and the LCDR3 of the VL3 of SEQ ID NO: 132.
18. The trispecific antibody or trispecific binding fragment of any one of embodiments 1-4 and 17, wherein the first antigen-binding arm that binds CD79b comprises the HCDR1, the HCDR2, the HCDR3, the LCDR1, the LCDR2 and the LCDR3 of SEQ ID NOs: 1, 2, 3, 4, 5 and 6, respectively;
the second antigen-binding arm that binds CD3 comprises the HCDR1, the HCDR2, the HCDR3, the LCDR1, the LCDR2 and the LCDR3 of SEQ ID NOs: 76, 77, 75, 79, 80 and 81, respectively; and
the third antigen-binding arm that binds CD20 comprises the HCDR1, the HCDR2, the HCDR3, the LCDR1, the LCDR2 and the LCDR3 of SEQ ID NOs: 121, 122, 123, 124, 119 and 125, respectively.
19. The trispecific antibody or trispecific binding fragment of any one of embodiments 1-4, 17, and 18, wherein the first antigen-binding arm that binds CD79b comprises the VH1 of SEQ ID NO: 35 and the VL1 of SEQ ID NO: 37;
the second antigen-binding arm that binds CD3 comprises the VH2 of SEQ ID NO: 101 and the VL2 of SEQ ID NO: 99; and
the third antigen-binding arm that binds CD20 comprises the VH3 of SEQ ID NO: 130 and the VL3 of SEQ ID NO: 132.
20. The trispecific antibody or trispecific binding fragment thereof of any one of embodiments 1-19, wherein the first antigen-binding arm and second antigen-binding arm each comprise a Fragment crystallizable (Fc) domain.
21. The trispecific antibody or trispecific binding fragment thereof of embodiment 20, wherein the Fc domains comprise one or more mutations which promote heterodimerization of the Fc domains.
22. The trispecific antibody or trispecific binding fragment of embodiment 21, wherein the mutations are selected from T366S, L368A, T366W and Y407V (EU numbering).
23. The trispecific antibody or trispecific binding fragment of any one of embodiments 20-22, wherein the Fc domains further comprise one or more mutations which reduce Fc binding to a Fcγ receptor.
24. The trispecific antibody or trispecific binding fragment of embodiment 23, wherein the Fcγ receptor is FcγRI, FcγRIIA, FcγRIIB, FcγRIIIA, and/or FcγRIIIB.
25. The trispecific antibody or trispecific binding fragment of embodiment 23 or 24, wherein the Fc domains comprise one or more mutations selected from L234A, L235A, and D265S (EU numbering).
26. The trispecific antibody or trispecific binding fragment of any one of embodiments 21-25, wherein the Fc domains further comprise one or more mutations which reduce Fc binding to protein A.
27. The trispecific antibody or trispecific binding fragment of embodiment 26, wherein the Fc domain comprises mutations H435R and/or Y436F (EU numbering).
28. The trispecific antibody or trispecific binding fragment of any one of embodiments 1 to 27, wherein the first antigen-binding arm specifically binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
29. The trispecific antibody or trispecific binding fragment of embodiment 28, wherein the first antigen-binding arm specifically binds to CD79b with an affinity of about 1×10⁻¹¹-1×10⁻⁹ M.
30. The trispecific antibody or trispecific binding fragment of any one of embodiments 1 to 29, wherein the second antigen-binding arm specifically binds to residues 54-58 (GSEIL; SEQ ID NO: 257), residues 74-75 (NI), and/or residues 100-105 (PRGSKP; SEQ ID NO: 258) of human CD3ε.
31. A trispecific antibody, or a trispecific binding fragment thereof, comprising a first antigen-binding arm that binds to an epitope on cluster of differentiation 79B protein (CD79b), a second antigen-binding arm that binds to an epitope on cluster of differentiation 3 (CD3), and a third antigen-binding arm that binds to an epitope on cluster of differentiation 20 (CD20),
wherein the first antigen-binding arm comprises a heavy chain (HC1) polypeptide and a light chain (LC) polypeptide; and
wherein the trispecific antibody, or a trispecific binding fragment thereof, comprise a single polypeptide comprising the second antigen-binding arm and the third antigen-binding arm, optionally wherein the trispecific antibody, or trispecific binding fragment thereof is in accordance with any one of embodiments 1-30
32. The trispecific antibody or trispecific binding fragment of embodiment 31, wherein the HC1 of the first antigen-binding arm comprises the amino acid sequence of SEQ ID NO: 172, 176, 180, or 191.
33. The trispecific antibody or trispecific binding fragment of embodiment 32, wherein the LC of the first antigen-binding arm comprises the amino acid sequence of SEQ ID NO: 174, 178 or 182.
34. The trispecific antibody or trispecific binding fragment of any one of embodiment 31-33, wherein
   a) the HC1 comprises the amino acid sequence of SEQ ID NO: 172 and the LC comprises the amino acid sequence of SEQ ID NO: 174;
   b) the HC1 comprises the amino acid sequence of SEQ ID NO: 176 and the LC comprises the amino acid sequence of SEQ ID NO: 178;
   c) the HC1 comprises the amino acid sequence of SEQ ID NO: 180 and the LC comprises the amino acid sequence of SEQ ID NO: 182;
   d) the HC1 comprises the amino acid sequence of SEQ ID NO: 191 and the LC comprises the amino acid sequence of SEQ ID NO: 182.
35. The trispecific antibody or trispecific binding fragment of embodiment 34, wherein the polypeptide comprising the second antigen-binding arm and the third antigen-binding arm comprises an the amino acid sequence of SEQ ID NO: 142, 144, 148, 150, 152, 154, 156, 158, 160, 162, 166, 168, or 170.
36. The trispecific antibody or trispecific binding fragment of embodiment 31-35, wherein
   a) the first antigen-binding arm comprises an HC1 comprising the amino acid sequence of SEQ ID NO: 172, and LC comprising the amino acid sequence of SEQ ID NO: 174, and the polypeptide comprising the second antigen-binding arm and the third antigen-binding arm comprises the amino acid sequence of SEQ ID NO: 142;
   b) the first antigen-binding arm comprises an HC1 comprising the amino acid sequence of SEQ ID NO: 176, the LC comprising the amino acid sequence of SEQ ID NO: 178, and the polypeptide comprising the second antigen-binding arm and the third antigen-binding arm comprises the amino acid sequence of SEQ ID NO: 142;
   c) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 142
   d) the first antigen-binding arm comprises an HC1 comprising the amino acid sequence of SEQ ID NO: 172, the LC comprising the amino acid sequence of SEQ ID NO: 174, and the polypeptide comprising the second antigen-binding arm and the third antigen-binding arm comprises the amino acid sequence of SEQ ID NO: 144;
   e) the first antigen-binding arm comprises an HC1 comprising the amino acid sequence of SEQ ID NO: 176, the LC comprising the amino acid sequence of SEQ ID NO: 178, and the polypeptide comprising the second antigen-binding arm and the third antigen-binding arm comprises the amino acid sequence of SEQ ID NO: 144;
   f) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 144;
   g) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 148;
   h) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 150;
   i) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 152;
   j) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 154;
   k) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 156;
   l) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 158;
   m) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 160;
   n) the first antigen-binding arm comprises an HC1 comprising the amino acid sequence of SEQ ID NO: 180, the LC comprising the amino acid sequence of SEQ ID NO: 182, and the polypeptide comprising the second antigen-binding arm and the third antigen-binding arm comprises the amino acid sequence of SEQ ID NO: 162;
   o) the first antigen-binding arm comprises an HC1 comprising the amino acid sequence of SEQ ID NO: 191, the LC comprising the amino acid sequence of SEQ ID NO: 182, and the polypeptide comprising the second antigen-binding arm and the third antigen-binding arm comprises the amino acid sequence of SEQ ID NO: 166;
   p) the first antigen-binding arm comprises an HC1 comprising the amino acid sequence of SEQ ID NO: 172, the LC comprising the amino acid sequence of SEQ ID NO: 174, and the polypeptide comprising the second antigen-binding arm and the third antigen-binding arm comprises the amino acid sequence of SEQ ID NO: 168; or
   q) the first antigen-binding arm comprises an HC1 comprising the amino acid sequence of SEQ ID NO: 172, the LC comprising the amino acid sequence of SEQ ID NO: 174, and the polypeptide comprising the second antigen-binding arm and the third antigen-binding arm comprises the amino acid sequence of SEQ ID NO: 170.
37. The trispecific antibody or trispecific binding fragment of any one of embodiments 31-36, wherein the first antigen-binding arm comprises an HC1 comprising the amino acid sequence of SEQ ID NO: 172, and a LC comprising the amino acid sequence of SEQ ID NO: 174, and the polypeptide comprising the second antigen-binding arm and the third antigen-binding arm comprises the amino acid sequence of SEQ ID NO: 168.
38. The trispecific antibody or trispecific binding fragment of any one of embodiments 31-36, wherein the first antigen-binding arm comprises an HC1 comprising the amino acid sequence of SEQ ID NO: 172, and a LC comprising the amino acid sequence of SEQ ID NO: 174, and the polypeptide comprising the second antigen-binding arm and the third antigen-binding arm comprises the amino acid sequence of SEQ ID NO: 170.
39. The trispecific antibody or trispecific binding fragment of any one of embodiments 1-38, wherein the antibody or antigen-binding fragment thereof is an IgG1, IgG2, IgG3, or IgG4 (human) isotype.
40. The trispecific antibody or trispecific binding fragment of any of embodiments 1-39, wherein the antibody or antigen-binding fragment thereof is an IgG1 (human) isotype.
41. A trispecific antibody, or a trispecific binding fragment thereof, comprising a first antigen-binding arm that binds to an epitope on cluster of differentiation 79B protein (CD79b), a second antigen-binding arm that binds to an epitope on cluster of differentiation 3 (CD3), and a third antigen-binding arm that binds to an epitope on cluster of differentiation 20 (CD20),
wherein the first antigen-binding arm comprises a heavy chain (HC1) polypeptide and a light chain (LC) polypeptide;
wherein the trispecific antibody, or a trispecific binding fragment thereof, comprise a single polypeptide comprising the second antigen-binding arm and the third antigen-binding arm, and
wherein HC1 comprises the amino acid sequence of SEQ ID NO: 172, LC comprises the amino acid sequence of SEQ ID NO: 174, and the single polypeptide comprising the second antigen-binding arm and the third antigen-binding arm comprises the amino acid sequence of SEQ ID NO: 168, optionally wherein the trispecific antibody, or trispecific binding fragment thereof is in accordance with any one of embodiments 1-36
42. A trispecific antibody, or a trispecific binding fragment thereof, comprising a first antigen-binding arm that binds to an epitope on cluster of differentiation 79B protein (CD79b), a second antigen-binding arm that binds to an epitope on cluster of differentiation 3 (CD3), and a third antigen-binding arm that binds to an epitope on cluster of differentiation 20 (CD20),
wherein the first antigen-binding arm comprises a heavy chain (HC1) polypeptide and a light chain (LC) polypeptide;
wherein the trispecific antibody, or a trispecific binding fragment thereof, comprises a single polypeptide comprising the second antigen-binding arm and the third antigen-binding arm, and
wherein HC1 comprises the amino acid sequence of SEQ ID NO: 172, LC comprises the amino acid sequence of SEQ ID NO: 174, and the single polypeptide comprising the second antigen-binding arm and the third antigen-binding arm comprises the amino acid sequence of SEQ ID NO: 170, optionally wherein the trispecific antibody, or trispecific binding fragment thereof is in accordance with any one of embodiments 1-36.
43. A synthetic polynucleotide encoding the trispecific antibody or trispecific binding fragment of any one of embodiments 1 to 30.
44. The synthetic polynucleotide of embodiment 43, wherein the polynucleotide comprises a sequence encoding an antigen-binding arm that binds CD79b, said sequence comprising
a) a VH-encoding sequence of SEQ ID NO: 36 and a VL-encoding sequence of SEQ ID NO: 38 or 213;
b) a VH-encoding sequence of SEQ ID NO: 40 and a VL-encoding sequence of SEQ ID NO: 42;
c) a VH-encoding sequence of SEQ ID NO: 44 and a VL-encoding sequence of SEQ ID NO: 34;
d) a VH-encoding sequence of SEQ ID NO: 46 and a VL-encoding sequence of SEQ ID NO: 48 or 214;
e) a VH-encoding sequence of SEQ ID NO: 50 and a VL-encoding sequence of SEQ ID NO: 52;
f) a VH-encoding sequence of SEQ ID NO: 40 and a VL-encoding sequence of SEQ ID NO: 54;
g) a VH-encoding sequence of SEQ ID NO: 56 and a VL-encoding sequence of SEQ ID NO: 58;
h) a VH-encoding sequence of SEQ ID NO: 60 and a VL-encoding sequence of SEQ ID NO: 62;
i) a VH-encoding sequence of SEQ ID NO: 64 and a VL-encoding sequence of SEQ ID NO: 66;
j) a VH-encoding sequence of SEQ ID NO: 68 and a VL-encoding sequence of SEQ ID NO: 70; or
k) a VH-encoding sequence of SEQ ID NO: 72 and a VL-encoding sequence of SEQ ID NO: 74.
45. The synthetic polynucleotide of embodiment 43 or 44, wherein the polynucleotide comprises a sequence encoding an antigen-binding arm that binds CD3, said sequence comprising
a) a VH-encoding sequence of SEQ ID NO: 98 and a VL-encoding sequence of SEQ ID NO: 100;
b) a VH-encoding sequence of SEQ ID NO: 102 and a VL-encoding sequence of SEQ ID NO: 100;
c) a VH-encoding sequence of SEQ ID NO: 104 and a VL-encoding sequence of SEQ ID NO: 100;
d) a VH-encoding sequence of SEQ ID NO: 106 and a VL-encoding sequence of SEQ ID NO: 100; or
e) a VH-encoding sequence of SEQ ID NO: 108 and a VL-encoding sequence of SEQ ID NO: 110.
46. The synthetic polynucleotide of any one of embodiments 43-45, wherein the polynucleotide comprises a sequence encoding an antigen-binding arm that binds CD20, said sequence comprising:
a) a VH-encoding sequence of SEQ ID NO: 127 and a VL-encoding sequence of SEQ ID NO: 129;
b) a VH-encoding sequence of SEQ ID NO: 131 and a VL-encoding sequence of SEQ ID NO: 133;
c) a VH-encoding sequence of SEQ ID NO: 135 and a VL-encoding sequence of SEQ ID NO: 137; or
d) a VH-encoding sequence of SEQ ID NO: 139 and a VL-encoding sequence of SEQ ID NO: 141.
47. A synthetic polynucleotide encoding the trispecific antibody or trispecific binding fragment of any one of embodiments 31 to 42.
48. The synthetic polynucleotide of embodiment 47, wherein the polynucleotide comprises a sequence encoding an HC1 comprising the nucleotide sequence of SEQ ID NO: 173, 177, 181, or 192.
49. The synthetic polynucleotide of any one of embodiments 47-48, wherein the polynucleotide comprises a sequence encoding a LC comprising the nucleotide sequence of SEQ ID NO: 175, 179, 183 or 188.
50. The synthetic polynucleotide of embodiment any one of embodiments 47-49, comprising
a) an HC1-encoding sequence of SEQ ID NO: 173 and a LC-encoding sequence of SEQ ID NO: 175;
b) an HC1-encoding sequence of SEQ ID NO: 177, and a LC-encoding sequence of SEQ ID NO: 179;
c) an HC1-encoding sequence of SEQ ID NO: 181, and a LC-encoding sequence of SEQ ID NO: 183;
d) an HC1-encoding sequence of SEQ ID NO: 181, and a LC-encoding sequence of SEQ ID NO: 188;
e) an HC1-encoding sequence of SEQ ID NO: 192, and a LC-encoding sequence of SEQ ID NO: 183.
51. The synthetic polynucleotide of any one of embodiments 47-50, wherein the polynucleotide comprises a sequence encoding the single polypeptide comprising the second antigen-binding arm and the third antigen-binding arm, said sequence comprises the nucleotide sequence of SEQ ID NO: 143, 145, 149, 151, 153, 155, 157, 159, 161, 163, 167, 169, or 171.
52. The synthetic polynucleotide of any one of embodiments 47-51, comprising
a) an HC1-encoding sequence of SEQ ID NO: 173, a LC-encoding sequence of SEQ ID NO: 175, and a sequence of SEQ ID NO: 143 which encodes the single polypeptide comprising the second antigen-binding arm and the third antigen-binding arm;
b) an HC1-encoding sequence of SEQ ID NO: 177, a LC-encoding sequence of SEQ ID NO: 179, and a sequence of SEQ ID NO: 143 which encodes the single polypeptide comprising the second antigen-binding arm and the third antigen-binding arm;
c) an HC1-encoding sequence of SEQ ID NO: 181, a LC-encoding sequence of SEQ ID NO: 183, and a sequence of SEQ ID NO: 143 which encodes the single polypeptide comprising the second antigen-binding arm and the third antigen-binding arm;
d) an HC1-encoding sequence of SEQ ID NO: 173, a LC-encoding sequence of SEQ ID NO: 175, and a sequence of SEQ ID NO: 145 which encodes the single polypeptide comprising the second antigen-binding arm and the third antigen-binding arm;
e) an HC1-encoding sequence of SEQ ID NO: 177, a LC-encoding sequence of SEQ ID NO: 179, and a sequence of SEQ ID NO: 145 which encodes the single polypeptide comprising the second antigen-binding arm and the third antigen-binding arm;
f) an HC1-encoding sequence of SEQ ID NO: 181, a LC-encoding sequence of SEQ ID NO: 183, and a sequence of SEQ ID NO: 145 which encodes the single polypeptide comprising the second antigen-binding arm and the third antigen-binding arm;
g) an HC1-encoding sequence of SEQ ID NO: 181, a LC-encoding sequence of SEQ ID NO: 188, and a sequence of SEQ ID NO: 149 which encodes single polypeptide comprising the second antigen-binding arm and the third antigen-binding arm;
h) an HC1-encoding sequence of SEQ ID NO: 181, a LC-encoding sequence of SEQ ID NO: 188, and a sequence of SEQ ID NO: 151 which encodes the single polypeptide comprising the second antigen-binding arm and the third antigen-binding arm;
i) an HC1-encoding sequence of SEQ ID NO: 181, a LC-encoding sequence of SEQ ID NO: 188, and a sequence of SEQ ID NO: 153 which encodes the single polypeptide comprising the second antigen-binding arm and the third antigen-binding arm;
j) an HC1-encoding sequence of SEQ ID NO: 181, a LC-encoding sequence of SEQ ID NO: 188, and a sequence of SEQ ID NO: 155 which encodes the single polypeptide comprising the second antigen-binding arm and the third antigen-binding arm;
k) an HC1-encoding sequence of SEQ ID NO: 181, a LC-encoding sequence of SEQ ID NO: 188, and a sequence of SEQ ID NO: 157 which encodes the single polypeptide comprising the second antigen-binding arm and the third antigen-binding arm;
l) an HC1-encoding sequence of SEQ ID NO: 181, a LC-encoding sequence of SEQ ID NO: 188, and a sequence of SEQ ID NO: 159 which encodes the single polypeptide comprising the second antigen-binding arm and the third antigen-binding arm;
m) an HC1-encoding sequence of SEQ ID NO: 181, a LC-encoding sequence of SEQ ID NO: 188, and a sequence of SEQ ID NO: 161 which encodes the single polypeptide comprising the second antigen-binding arm and the third antigen-binding arm;
n) an HC1-encoding sequence of SEQ ID NO: 181, a LC-encoding sequence of SEQ ID NO: 188, and a sequence of SEQ ID NO: 163 which encodes the single polypeptide comprising the second antigen-binding arm and the third antigen-binding arm;
o) an HC1-encoding sequence of SEQ ID NO: 192, a LC-encoding sequence of SEQ ID NO: 183, and a sequence of SEQ ID NO: 167 which encodes the single polypeptide comprising the second antigen-binding arm and the third antigen-binding arm;
p) an HC1-encoding sequence of SEQ ID NO: 173, a LC-encoding sequence of SEQ ID NO: 175, and a sequence of SEQ ID NO: 169 which encodes the single polypeptide comprising the second antigen-binding arm and the third antigen-binding arm; or
q) an HC1-encoding sequence of SEQ ID NO: 173, a LC-encoding sequence of SEQ ID NO: 175, and a sequence of SEQ ID NO: 171 which encodes the single polypeptide comprising the second antigen-binding arm and the third antigen-binding arm.
53. The synthetic polynucleotide of any one of embodiments 47-52, comprising an HC1-encoding sequence of SEQ ID NO: 173, a LC-encoding sequence of SEQ ID NO: 175, and a sequence of SEQ ID NO: 169 which encodes the single polypeptide comprising the second antigen-binding arm and the third antigen-binding arm.
54. The synthetic polynucleotide of any one of embodiments 47-52, comprising an HC1-encoding sequence of SEQ ID NO: 173, a LC-encoding sequence of SEQ ID NO: 175, and a sequence of SEQ ID NO: 171 which encodes the single polypeptide comprising the second antigen-binding arm and the third antigen-binding arm.
55. A synthetic polynucleotide encoding a trispecific antibody, or a trispecific binding fragment thereof, said trispecific antibody or trispecific binding fragment comprising a first antigen-binding arm that binds to an epitope on cluster of differentiation 79B protein (CD79b), a second antigen-binding arm that binds to an epitope on cluster of differentiation 3 (CD3), and a third antigen-binding arm that binds to an epitope on cluster of differentiation 20 (CD20),
wherein the first antigen-binding arm comprises a heavy chain (HC1) polypeptide and a light chain (LC) polypeptide,
wherein the trispecific antibody, or a trispecific binding fragment thereof, comprise a single polypeptide comprising the second antigen-binding arm and the third antigen-binding arm, and
(a) wherein the polynucleotide comprises an HC1-encoding sequence of SEQ ID NO: 173, a LC-encoding sequence of SEQ ID NO: 175, and a sequence of SEQ ID NO: 169 which encodes the single polypeptide comprising the second antigen-binding arm and the third antigen-binding arm; or
(b) wherein the polynucleotide comprises an HC1-encoding sequence of SEQ ID NO: 173, a LC-encoding sequence of SEQ ID NO: 175, and a sequence of SEQ ID NO: 171 which encodes the single polypeptide comprising the second antigen-binding arm and the third antigen-binding arm; optionally wherein the polynucleotide is in accordance with any one of embodiments 43-54.
56. A pharmaceutical composition comprising the trispecific antibody or trispecific binding fragment of any one of embodiments 1 to 42, or the polynucleotide of any one of embodiments 43-55, and a pharmaceutically acceptable carrier.
57. The pharmaceutical composition of embodiment 56, wherein the pharmaceutical composition further comprises a second therapeutic agent.
58. The pharmaceutical composition of embodiment 57, wherein the second therapeutic agent comprises a chemotherapeutic agent, anti-CD20 agent, anti-CD19 agent, anti-CD22 agent, Bruton's tyrosine kinase (BTK) inhibitor, mucosa-associated lymphoid tissue lymphoma translocation protein 1 (MALT1) inhibitor, immunomodulatory imide drug (IMiD), pro apoptotic B cell lymphoma 2 (Bcl-2) family inhibitor, phosphoinositide 3-kinase (PI3K) inhibitor, immune checkpoint inhibitor, CD28 costimulatory bispecific antibody, CD137 costimulatory bispecific antibody, or a combination thereof.
59. An isolated cell expressing the trispecific antibody or trispecific binding fragment of any one of embodiments 1 to 42.
60. The cell of embodiment 59 wherein the cell is a hybridoma.
61. The cell of embodiment 59 wherein the antibody is recombinantly produced.
62. A method for treating cancer in a subject in need thereof, said method comprising administering to the subject a therapeutically effective amount of the trispecific antibody or trispecific binding fragment of any one of embodiments 1 to 42, the polynucleotide of any one of embodiments 43-55, or the pharmaceutical composition of any one of embodiments 56-58.
63. The method of embodiment 62, wherein the trispecific antibody or trispecific binding fragment, polynucleotide, or the pharmaceutical composition is administered for a time sufficient to treat the cancer.
64. A method for inhibiting growth or proliferation of a cancer cell, said method comprising administering to said cell an effective amount of the trispecific antibody or trispecific binding fragment of any one of embodiments 1 to 42, the polynucleotide of any one of embodiments 43-55, or the pharmaceutical composition of any one of embodiments 56-58, wherein said effective amount is sufficient to inhibit the growth or proliferation of said cancer cell.
65. The method of embodiment 64, wherein said cancer cell is in a subject and the trispecific antibody or trispecific binding fragment, polynucleotide, or the pharmaceutical composition is administered to the subject.
66. The method of embodiment 65, wherein said administration is conducted ex vivo.
67. A method of redirecting a T cell to CD79b and/or CD20-expressing cancer cells in a subject in need thereof, said method comprising administering to said subject a therapeutically effective amount of the trispecific antibody or trispecific binding fragment of any one of embodiments 1 to 42, the polynucleotide of any one of embodiments 43-55, or the pharmaceutical composition of any one of embodiments 56-58.
68. The method of embodiment 67, wherein the said therapeutically effective amount is sufficient to direct said T cell response to said cancer cells.
69. The method of any one of embodiments 62-68, wherein the cancer is a hematological cancer.
70. The method of embodiment 69 wherein the hematological cancer is a CD79b and/or CD20-expressing B cell cancer.
71. The method of embodiment 70 wherein the CD79b and/or CD20-expressing B cell cancer is a B-cell lymphoma or a non-Hodgkin lymphoma.
72. The method of embodiment 71 wherein the CD79b and/or CD20-expressing B cell cancer is a diffuse large B-cell lymphoma (DLBCL), mantle cell lymphoma (MCL), marginal zone lymphoma (MZL), follicular lymphoma (FL), chronic lymphocytic leukemia (CLL), or Waldenström macroglobulinemia (WM).
73. The method of any one of embodiments 62-72, wherein the cancer is relapsed, refractory, or malignant cancer, or any combination thereof.
74. The method of any one of embodiments 62-73 further comprising administering a second therapeutic agent.
75. The method of embodiment 74 wherein the second therapeutic agent is a surgery, chemotherapy, androgen deprivation therapy or radiation, anti-CD20 agent, anti-CD19 agent, anti-CD22 agent, Bruton's tyrosine kinase (BTK) inhibitor, mucosa-associated lymphoid tissue lymphoma translocation protein 1 (MALT1) inhibitor, immunomodulatory imide drug (IMiD), pro apoptotic B cell lymphoma 2 (Bcl-2) family inhibitor, phosphoinositide 3-kinase (PI3K) inhibitor, immune checkpoint inhibitor, CD28 costimulatory bispecific antibody, CD137 costimulatory bispecific antibody, or any combination thereof.
76. The method of any one of embodiments 62-63, 65 and 67-75, wherein the trispecific antibody or trispecific binding fragment, polynucleotide, or the pharmaceutical composition is administered intravenously, intramuscularly, intraperitoneally, and/or subcutaneously to the subject.
77. The method of any one of embodiments 62-63, 65 and 67-75, wherein the trispecific antibody or trispecific binding fragment, polynucleotide, or the pharmaceutical composition is administered subcutaneously to the subject.
78. A method for generating the trispecific antibody or trispecific binding fragment of any one of embodiments 1 to 42, wherein said method comprises culturing the cell of any one of embodiments 59 to 61 and isolating said trispecific antibody or trispecific binding fragment.
79. A kit comprising (i) the trispecific antibody or trispecific binding fragment of any one of embodiments 1 to 42 and/or the polynucleotide of any one of embodiments 43-55 and (ii) packaging for the same.
80. An antibody, or an antigen-binding fragment thereof, that binds to an epitope on Cluster of Differentiation 79B protein (CD79b), comprising:
a) a heavy chain complementarity determining region (HCDR) 1, a HCDR2 and a HCDR3 of a heavy chain variable domain (VH) of SEQ ID NO: 35 and a light chain complementarity determining region (LCDR) 1, a LCDR2 and a LCDR3 of a light chain variable domain (VL) of SEQ ID NO: 37;
b) the HCDR1, the HCDR2 and the HCDR3 of the VH of SEQ ID NO: 39 and the LCDR1, the LCDR2 and the LCDR3 of the VL of SEQ ID NO: 41;
c) the HCDR1, the HCDR2 and the HCDR3 of the VH of SEQ ID NO: 43 and the LCDR1, the LCDR2 and the LCDR3 of the VL of SEQ ID NO: 41;
d) the HCDR1, the HCDR2 and the HCDR3 of the VH of SEQ ID NO: 45 and the LCDR1, the LCDR2 and the LCDR3 of the VL of SEQ ID NO: 47;
e) the HCDR1, the HCDR2 and the HCDR3 of the VH of SEQ ID NO: 49 and the LCDR1, the LCDR2 and the LCDR3 of the VL of SEQ ID NO: 51;
f) the HCDR1, the HCDR2 and the HCDR3 of the VH of SEQ ID NO: 39 and the LCDR1, the LCDR2 and the LCDR3 of the VL of SEQ ID NO: 53;
g) the HCDR1, the HCDR2 and the HCDR3 of the VH of SEQ ID NO: 55 and the LCDR1, the LCDR2 and the LCDR3 of the VL of SEQ ID NO: 57;
h) the HCDR1, the HCDR2 and the HCDR3 of the VH of SEQ ID NO: 59 and the LCDR1, the LCDR2 and the LCDR3 of the VL of SEQ ID NO: 61;
i) the HCDR1, the HCDR2 and the HCDR3 of the VH of SEQ ID NO: 63 and the LCDR1, the LCDR2 and the LCDR3 of the VL of SEQ ID NO: 65;
j) the HCDR1, the HCDR2 and the HCDR3 of the VH of SEQ ID NO: 67 and the LCDR1, the LCDR2 and the LCDR3 of the VL of SEQ ID NO: 69; or
k) the HCDR1, the HCDR2 and the HCDR3 of the VH of SEQ ID NO: 71 and the LCDR1, the LCDR2 and the LCDR3 of the VL of SEQ ID NO: 73.
81. The antibody or antigen-binding fragment of embodiment 80, comprising the HCDR1, the HCDR2, the HCDR3, the LCDR1, the LCDR2 and the LCDR3 of:
a) SEQ ID NOs: 1, 2, 3, 4, 5 and 6, respectively;
b) SEQ ID NOs: 13, 8, 9, 10, 11 and 12, respectively;
c) SEQ ID NOs: 7, 8, 9, 10, 11 and 12, respectively;
d) SEQ ID NOs: 14, 15, 16, 17, 5 and 6, respectively;
e) SEQ ID NOs: 18, 8, 19, 20, 21 and 12, respectively;
f) SEQ ID NOs: 22, 23, 24, 25, 5 and 6, respectively;
g) SEQ ID NOs: 22, 26, 27, 28, 5 and 29, respectively; or
h) SEQ ID NOs: 30, 31, 32, 33, 5 and 6, respectively.
82. The antibody or antigen-binding fragment of embodiment 80 or 81, comprising
a) the VH of SEQ ID NO: 35 and the VL of SEQ ID NO: 37;
b) the VH of SEQ ID NO: 39 and the VL of SEQ ID NO: 41;
c) the VH of SEQ ID NO: 43 and the VL of SEQ ID NO: 41;
d) the VH of SEQ ID NO: 45 and the VL of SEQ ID NO: 47;
e) the VH of SEQ ID NO: 49 and the VL of SEQ ID NO: 51;
f) the VH of SEQ ID NO: 39 and the VL of SEQ ID NO: 53;
g) the VH of SEQ ID NO: 55 and the VL of SEQ ID NO: 57;
h) the VH of SEQ ID NO: 59 and the VL of SEQ ID NO: 61;
i) the VH of SEQ ID NO: 63 and the VL of SEQ ID NO: 65;
j) the VH of SEQ ID NO: 67 and the VL of SEQ ID NO: 69; or
k) the VH of SEQ ID NO: 71 and the VL of SEQ ID NO: 73.
83. The antibody or antigen-binding fragment of any one of embodiments 80-82, wherein the antibody or antigen-binding fragment specifically binds residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
84. The antibody or antigen-binding fragment of embodiment 83, wherein the antibody or antigen-binding fragment specifically binds to CD79b with an affinity of about 1×10⁻¹¹-1×10⁻⁹ M.
85. The antibody or antigen-binding fragment of any one of embodiments 80-84 wherein the antibody or antigen-binding fragment is a human antibody or antigen-binding fragment.
86. The antibody or antigen-binding fragment of any one of embodiments 80 to 85 wherein the antibody or antigen-binding fragment is recombinant.
87. The antigen binding fragment of any one of embodiments 80 to 86 wherein the antigen binding fragment is a diabody, a Fab, Fab′, a F(ab′)2, a Fv, a scFv, a Fd, a disulfide stabilized Fv fragment (dsFv), or a disulfide stabilized diabody (ds diabody).
88. The antibody or antigen-binding fragment of any one of embodiments 80 to 87 wherein the antibody or antigen-binding fragment thereof is an IgG1, IgG2, IgG3, or IgG4 (human) isotype.
89. The antibody or antigen-binding fragment of any of embodiments 80 to 88, wherein the antibody or antigen-binding fragment thereof is an IgG1 or an IgG4 (human) isotype.
90. A synthetic polynucleotide encoding the antibody or antigen-binding fragment of any one of embodiments 80 to 89.
91. The synthetic polynucleotide of embodiment 90, comprising
a) a VH-encoding sequence of SEQ ID NO: 36 and a VL-encoding sequence of SEQ ID NO: 38 or 213;
b) a VH-encoding sequence of SEQ ID NO: 40 and a VL-encoding sequence of SEQ ID NO: 42;
c) a VH-encoding sequence of SEQ ID NO: 44 and a VL-encoding sequence of SEQ ID NO: 34;
d) a VH-encoding sequence of SEQ ID NO: 46 and a VL-encoding sequence of SEQ ID NO: 48 or 214;
e) a VH-encoding sequence of SEQ ID NO: 50 and a VL-encoding sequence of SEQ ID NO: 52;
f) a VH-encoding sequence of SEQ ID NO: 40 and a VL-encoding sequence of SEQ ID NO: 54;
g) a VH-encoding sequence of SEQ ID NO: 56 and a VL-encoding sequence of SEQ ID NO: 58;
h) a VH-encoding sequence of SEQ ID NO: 60 and a VL-encoding sequence of SEQ ID NO: 62;
i) a VH-encoding sequence of SEQ ID NO: 64 and a VL-encoding sequence of SEQ ID NO: 66;
j) a VH-encoding sequence of SEQ ID NO: 68 and a VL-encoding sequence of SEQ ID NO: 70; or
k) a VH-encoding sequence of SEQ ID NO: 72 and a VL-encoding sequence of SEQ ID NO: 74.
92. A pharmaceutical composition comprising the antibody or antigen-binding fragment of any one of embodiments 80 to 89, or the polynucleotide of embodiment 90 or 91, and a pharmaceutically acceptable carrier.
93. The pharmaceutical composition of embodiment 92, wherein the pharmaceutical composition further comprises a second therapeutic agent.
94. The pharmaceutical composition of embodiment 93, wherein the second therapeutic agent comprises a chemotherapeutic agent, anti-CD20 agent, anti-CD19 agent, anti-CD22 agent, Bruton's tyrosine kinase (BTK) inhibitor, mucosa-associated lymphoid tissue lymphoma translocation protein 1 (MALT1) inhibitor, immunomodulatory imide drug (IMiD), pro apoptotic B cell lymphoma 2 (Bcl-2) family inhibitor, phosphoinositide 3-kinase (PI3K) inhibitor, immune checkpoint inhibitor, CD28 costimulatory bispecific antibody, CD137 costimulatory bispecific antibody, or a combination thereof.
95. An isolated cell expressing the antibody or antigen-binding fragment of any one of embodiments 80-89.
96. The cell of embodiment 95 wherein the cell is a hybridoma.
97. The cell of embodiment 95 wherein the antibody is recombinantly produced.
98. A method for treating cancer in a subject in need thereof, said method comprising administering to the subject a therapeutically effective amount of the antibody or antigen-binding fragment of any one of embodiments 80 to 89 or the polynucleotide of embodiment 90 or 91, or the pharmaceutical composition of any one of embodiments 92-98.
99. The method of embodiment 98, wherein the antibody or antigen-binding fragment or the pharmaceutical composition is administered for a time sufficient to treat the cancer.
100. A method for inhibiting growth or proliferation of a cancer cell, said method comprising administering to said cell an effective amount of the antibody or antigen-binding fragment of any one of embodiments 80 to 89, or the polynucleotide of embodiment 90 or 91, or the pharmaceutical composition of any one of embodiments 92-98, wherein said effective amount is sufficient to inhibit the growth or proliferation of said cancer cell.
101. The method of embodiment 100, wherein said cancer cell is in a subject and the antibody or antigen-binding fragment or the pharmaceutical composition is administered to the subject.
102. The method of embodiment 101, wherein said administration is conducted ex vivo.
103. The method of any one of embodiments 98-102 wherein the cancer is a hematological cancer.
104. The method of embodiment 103 wherein the hematological cancer is a CD79b-expressing B cell cancer.
105. The method of embodiment 104 wherein the CD79b-expressing B cell cancer is a B-cell lymphoma or a non-Hodgkin lymphoma.
106. The method of embodiment 105 wherein the CD79b-expressing B cell cancer is a diffuse large B-cell lymphoma (DLBCL), mantle cell lymphoma (MCL), marginal zone lymphoma (MZL), follicular lymphoma (FL), chronic lymphocytic leukemia (CLL), or Waldenström macroglobulinemia (WM).
107. The method of any one of embodiments 98-106, wherein the cancer is relapsed, refractory, or malignant cancer, or any combination thereof.
108. The method of any one of embodiments 98-107 further comprising administering a second therapeutic agent.
109. The method of embodiment 108 wherein the second therapeutic agent is a surgery, chemotherapy, androgen deprivation therapy or radiation, anti-CD20 agent, anti-CD19 agent, anti-CD22 agent, Bruton's tyrosine kinase (BTK) inhibitor, mucosa-associated lymphoid tissue lymphoma translocation protein 1 (MALT1) inhibitor, immunomodulatory imide drug (IMiD), pro apoptotic B cell lymphoma 2 (Bcl-2) family inhibitor, phosphoinositide 3-kinase (PI3K) inhibitor, immune checkpoint inhibitor, CD28 costimulatory bispecific antibody, CD137 costimulatory bispecific antibody, or any combination thereof.
110. The method of any one of embodiments 98-99, 101 and 103-109, wherein the antibody or antigen-binding fragment, polynucleotide, or the pharmaceutical composition is administered intravenously, intramuscularly, intraperitoneally, and/or subcutaneously to the subject.
111. The method of any one of embodiments 98-99, 101 and 103-110, wherein the antibody or antigen-binding fragment, polynucleotide, or the pharmaceutical composition is administered subcutaneously to the subject.
112. A method for generating the antibody or antigen-binding fragment of any one of embodiments 80 to 89, wherein said method comprises culturing the cell of any one of embodiments 95 to 97 and isolating said antibody or antigen-binding fragment.
113. A kit comprising (i) the antibody or antigen-binding fragment of any one of embodiments 80 to 89 and/or a polynucleotide of embodiment 90 or 91 and (ii) packaging for the same.
114. A bispecific antibody, or a bispecific binding fragment thereof, comprising:
(a) a first antigen-binding arm comprising a first heavy chain variable domain (VH1) and a first light chain variable domain (VL1);
(b) a second antigen-binding arm comprising a second heavy chain variable domain (VH2) and a second light chain variable domain (VL2);
wherein the first antigen-binding arm binds to an epitope on cluster of differentiation 79B protein (CD79b), and the second antigen-binding arm binds to an epitope on is cluster of differentiation 3 (CD3).
115. The bispecific antibody or bispecific binding fragment of any one of embodiments 114, wherein the VH1 and VL1 of first antigen-binding arm are present in a diabody, a Fab, Fab′, a F(ab′)2, a Fv, a scFv, a Fd, a disulfide stabilized Fv fragment (dsFv), or a disulfide stabilized diabody (ds diabody), optionally a Fab.
116. The bispecific antibody or bispecific binding fragment of embodiment 114-115, wherein the VH2 and VL2 of the second antigen-binding arm are present in a diabody, a Fab, Fab′, a F(ab′)2, a Fv, a scFv, a Fd, a disulfide stabilized Fv fragment (dsFv), or a disulfide stabilized diabody (ds diabody), optionally a scFv.
117. The bispecific antibody or bispecific binding fragment of any one of embodiments 114-116, wherein the first antigen-binding arm that binds CD79b comprises:
a) a heavy chain complementarity determining region (HCDR) 1, a HCDR2 and a HCDR3 of a heavy chain variable domain (VH) of SEQ ID NO: 35 and a light chain complementarity determining region (LCDR) 1, a LCDR2 and a LCDR3 of a light chain variable domain (VL) of SEQ ID NO: 37;
b) the HCDR1, the HCDR2 and the HCDR3 of the VH of SEQ ID NO: 39 and the LCDR1, the LCDR2 and the LCDR3 of the VL of SEQ ID NO: 41;
c) the HCDR1, the HCDR2 and the HCDR3 of the VH of SEQ ID NO: 43 and the LCDR1, the LCDR2 and the LCDR3 of the VL of SEQ ID NO: 41;
d) the HCDR1, the HCDR2 and the HCDR3 of the VH of SEQ ID NO: 45 and the LCDR1, the LCDR2 and the LCDR3 of the VL of SEQ ID NO: 47;
e) the HCDR1, the HCDR2 and the HCDR3 of the VH of SEQ ID NO: 49 and the LCDR1, the LCDR2 and the LCDR3 of the VL of SEQ ID NO: 51;
f) the HCDR1, the HCDR2 and the HCDR3 of the VH of SEQ ID NO: 39 and the LCDR1, the LCDR2 and the LCDR3 of the VL of SEQ ID NO: 53;
g) the HCDR1, the HCDR2 and the HCDR3 of the VH of SEQ ID NO: 55 and the LCDR1, the LCDR2 and the LCDR3 of the VL of SEQ ID NO: 57;
h) the HCDR1, the HCDR2 and the HCDR3 of the VH of SEQ ID NO: 59 and the LCDR1, the LCDR2 and the LCDR3 of the VL of SEQ ID NO: 61;
i) the HCDR1, the HCDR2 and the HCDR3 of the VH of SEQ ID NO: 63 and the LCDR1, the LCDR2 and the LCDR3 of the VL of SEQ ID NO: 65;
j) the HCDR1, the HCDR2 and the HCDR3 of the VH of SEQ ID NO: 67 and the LCDR1, the LCDR2 and the LCDR3 of the VL of SEQ ID NO: 69; or
k) the HCDR1, the HCDR2 and the HCDR3 of the VH of SEQ ID NO: 71 and the LCDR1, the LCDR2 and the LCDR3 of the VL of SEQ ID NO: 73.
118. The bispecific antibody or bispecific binding fragment of any one of embodiments 114-117, wherein the first antigen-binding arm that binds CD79b comprises the HCDR1, the HCDR2, the HCDR3, the LCDR1, the LCDR2 and the LCDR3 of:
a) SEQ ID NOs: 1, 2, 3, 4, 5 and 6, respectively;
b) SEQ ID NOs: 13, 8, 9, 10, 11 and 12, respectively;
c) SEQ ID NOs: 7, 8, 9, 10, 11 and 12, respectively;
d) SEQ ID NOs: 14, 15, 16, 17, 5 and 6, respectively;
e) SEQ ID NOs: 18, 8, 19, 20, 21 and 12, respectively;
f) SEQ ID NOs: 22, 23, 24, 25, 5 and 6, respectively;
g) SEQ ID NOs: 22, 26, 27, 28, 5 and 29, respectively; or
h) SEQ ID NOs: 30, 31, 32, 33, 5 and 6, respectively.
119. The bispecific antibody or bispecific binding fragment of any one of embodiments 114-118, wherein the first antigen-binding arm that binds CD79b comprises:
a) the VH of SEQ ID NO: 35 and the VL of SEQ ID NO: 37;
b) the VH of SEQ ID NO: 39 and the VL of SEQ ID NO: 41;
c) the VH of SEQ ID NO: 43 and the VL of SEQ ID NO: 41;
d) the VH of SEQ ID NO: 45 and the VL of SEQ ID NO: 47;
e) the VH of SEQ ID NO: 49 and the VL of SEQ ID NO: 51;
f) the VH of SEQ ID NO: 39 and the VL of SEQ ID NO: 53;
g) the VH of SEQ ID NO: 55 and the VL of SEQ ID NO: 57;
h) the VH of SEQ ID NO: 59 and the VL of SEQ ID NO: 61;
i) the VH of SEQ ID NO: 63 and the VL of SEQ ID NO: 65;
j) the VH of SEQ ID NO: 67 and the VL of SEQ ID NO: 69; or
k) the VH of SEQ ID NO: 71 and the VL of SEQ ID NO: 73.
120. The bispecific antibody or bispecific binding fragment of any one of embodiments 114-119, wherein the second antigen-binding arm that binds CD3 comprises:
a) the HCDR1, the HCDR2 and the HCDR3 of the VH of SEQ ID NO: 97 and the LCDR1, the LCDR2 and the LCDR3 of the VL of SEQ ID NO: 99;
b) the HCDR1, the HCDR2 and the HCDR3 of the VH of SEQ ID NO: 101 and the LCDR1, the LCDR2 and the LCDR3 of the VL of SEQ ID NO: 99;
c) the HCDR1, the HCDR2 and the HCDR3 of the VH of SEQ ID NO: 103 and the LCDR1, the LCDR2 and the LCDR3 of the VL of SEQ ID NO: 99;
d) the HCDR1, the HCDR2 and the HCDR3 of the VH of SEQ ID NO: 105 and the LCDR1, the LCDR2 and the LCDR3 of the VL of SEQ ID NO: 99; or
e) the HCDR1, the HCDR2 and the HCDR3 of the VH of SEQ ID NO: 107 and the LCDR1, the LCDR2 and the LCDR3 of the VL of SEQ ID NO: 109.
121. The bispecific antibody or bispecific binding fragment of any one of embodiments 114-120, wherein the second antigen-binding arm that binds CD3 comprises the HCDR1, the HCDR2, the HCDR3, the LCDR1, the LCDR2 and the LCDR3 of:
a) SEQ ID NOs: 76, 77, 78, 79, 80 and 81, respectively;
b) SEQ ID NOs: 76, 77, 75, 79, 80 and 81, respectively;
c) SEQ ID NOs: 76, 77, 82, 79, 80 and 81, respectively; or
d) SEQ ID NOs: 83, 84, 85, 86, 87 and 88, respectively.
122. The bispecific antibody or bispecific binding fragment of any one of embodiments 114-121, wherein the second or third antigen-binding arm that binds CD3 comprises:
a) the VH of SEQ ID NO: 97 and the VL of SEQ ID NO: 99;
b) the VH of SEQ ID NO: 101 and the VL of SEQ ID NO: 99;
c) the VH of SEQ ID NO: 103 and the VL of SEQ ID NO: 99;
d) the VH of SEQ ID NO: 105 and the VL of SEQ ID NO: 99; or
e) the VH of SEQ ID NO: 107 and the VL of SEQ ID NO: 109.
123. The bispecific antibody or bispecific binding fragment thereof of any one of embodiments 114-122, the first antigen-binding arm and second antigen-binding arm each comprise a Fragment crystallizable (Fc) domain.
124. The bispecific antibody or bispecific binding fragment of embodiment 123, wherein the Fc domains of the first antigen-binding arm and second antigen-binding arm comprise one or more different mutations which promote heterodimerization of the Fc domains.
125. The bispecific antibody or bispecific binding fragment of embodiment 124, wherein the mutations are selected from T366S, L368A, T366W and Y407V (EU numbering).
126. The bispecific antibody or bispecific binding fragment of any one of embodiments 123-125, wherein the Fc domains of the first antigen-binding arm and/or the second antigen-binding arm further comprise one or more mutations which reduce Fc binding to a Fcγ receptor.
127. The bispecific antibody or bispecific binding fragment of embodiment 126, wherein the Fcγ receptor is FcγRI, FcγRIIA, FcγRIIB, FcγRIIIA, and/or FcγRIIIB.
128. The bispecific antibody or bispecific binding fragment of embodiment 126 or 127, wherein the Fc domains of the first antigen-binding arm and/or the second antigen-binding arm each comprise one or more mutations selected from L234A, L235A, and D265S (EU numbering).
129. The bispecific antibody or bispecific binding fragment of embodiment 128, wherein the Fe domains of the first antigen-binding arm and the second antigen-binding arm each comprise mutations L234A, L235A, and D265S (EU numbering).
130. The bispecific antibody or bispecific binding fragment of any one of embodiments 124-129, wherein the Fc domains of the first antigen-binding arm or the second antigen-binding arm further comprise one or more mutations which reduce Fc binding to protein A.
131. The bispecific antibody or bispecific binding fragment of embodiment 130, wherein the Fc domains of the first antigen-binding arm or the second antigen-binding arm comprise mutations H435R and/or Y436F (EU numbering).
132. The bispecific antibody or bispecific binding fragment of embodiment 131, wherein the Fc domain of the first antigen-binding arm comprises mutations H435R and Y436F (EU numbering).
133. The bispecific antibody or bispecific binding fragment of any one of embodiments 114-132, wherein the first antigen-binding arm specifically binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
134. The bispecific antibody or bispecific binding fragment of embodiment 133, wherein the first antigen-binding arm specifically binds to CD79b with an affinity of about 1×10⁻¹¹-1×10⁻⁹ M.
135. The bispecific antibody or bispecific binding fragment of any one of embodiments 114-134, wherein the second antigen-binding arm specifically binds to residues 54-58 (GSEIL; SEQ ID NO: 257), residues 74-75 (NI), and/or residues 100-105 (PRGSKP; SEQ ID NO: 258) of human CD3ε.
136. A bispecific antibody, or a bispecific binding fragment, comprising a first antigen-binding arm that binds to an epitope on cluster of differentiation 79B protein (CD79b), a second antigen-binding arm that binds to an epitope on cluster of differentiation 3 (CD3),
wherein the first antigen-binding arm comprises a heavy chain (HC1) polypeptide and a light chain (LC) polypeptide; and
the second antigen-binding arm comprises a second antigen-binding arm polypeptide.
137. The bispecific antibody or bispecific binding fragment of embodiment 136, wherein the HC1 comprises the amino acid sequence of SEQ ID NO: 172, 176, or 180.
138. The bispecific antibody or bispecific binding fragment of embodiment 136 or 137, wherein the LC comprises the amino acid sequence of SEQ ID NO: 174, 178, or 182.
139. The bispecific antibody or bispecific binding fragment of any one of embodiments 136-138, wherein
a) the HC1 comprises the amino acid sequence of SEQ ID NO: 172 and the LC comprises the amino acid sequence of SEQ ID NO: 174;
b) the HC1 comprises the amino acid sequence of SEQ ID NO: 176 and the LC comprises the amino acid sequence of SEQ ID NO: 178;
c) the HC1 comprises the amino acid sequence of SEQ ID NO: 180 and the LC comprises the amino acid sequence of SEQ ID NO: 182.
140. The bispecific antibody or bispecific binding fragment of any one of embodiments 136-139, wherein the second antigen-binding arm polypeptide comprises the amino acid sequence of SEQ ID NO: 164 or 189.
141. The bispecific antibody or bispecific binding fragment of any one of embodiments 136-140, wherein
a) the HC1 comprises the amino acid sequence of SEQ ID NO: 172, the LC comprises the amino acid sequence of SEQ ID NO: 174, and the second antigen-binding arm polypeptide comprises the amino acid sequence of SEQ ID NO: 164;
b) the HC1 comprises the amino acid sequence of SEQ ID NO: 176, the LC comprises the amino acid sequence of SEQ ID NO: 178, and the second antigen-binding arm polypeptide comprises the amino acid sequence of SEQ ID NO: 164;
c) the HC1 comprises the amino acid sequence of SEQ ID NO: 180, the LC comprises the amino acid sequence of SEQ ID NO: 182, and the second antigen-binding arm polypeptide comprises the amino acid sequence of SEQ ID NO: 164;
d) the HC1 comprises the amino acid sequence of SEQ ID NO: 172, the LC comprises the amino acid sequence of SEQ ID NO: 174, and the second antigen-binding arm polypeptide comprises the amino acid sequence of SEQ ID NO: 189;
e) the HC1 comprises the amino acid sequence of SEQ ID NO: 176, the LC comprises the amino acid sequence of SEQ ID NO: 178, and the second antigen-binding arm polypeptide comprises the amino acid sequence of SEQ ID NO: 189; or
f) the HC1 comprises the amino acid sequence of SEQ ID NO: 180, the LC comprises the amino acid sequence of SEQ ID NO: 182, and the second antigen-binding arm polypeptide comprises the amino acid sequence of SEQ ID NO: 189.
142. The bispecific antibody or bispecific binding fragment of any one of embodiments 114 to 141, wherein the antibody or antigen-binding fragment thereof is an IgG1, IgG2, IgG3, or IgG4 isotype.
143. The bispecific antibody or bispecific binding fragment of any of embodiments 114 to 142, wherein the antibody or antigen-binding fragment thereof is an IgG1 isotype.
144. An synthetic polynucleotide encoding the bispecific antibody or bispecific binding fragment of any one of embodiments 114 to 135.
145. The synthetic polynucleotide of embodiment 144, wherein the polynucleotide comprises a sequence encoding an antigen-binding arm that binds CD79b, said sequence comprising
a) a VH-encoding sequence of SEQ ID NO: 36 and a VL-encoding sequence of SEQ ID NO: 38 or 213;
b) a VH-encoding sequence of SEQ ID NO: 40 and a VL-encoding sequence of SEQ ID NO: 42;
c) a VH-encoding sequence of SEQ ID NO: 44 and a VL-encoding sequence of SEQ ID NO: 34;
d) a VH-encoding sequence of SEQ ID NO: 46 and a VL-encoding sequence of SEQ ID NO: 48 or 214;
e) a VH-encoding sequence of SEQ ID NO: 50 and a VL-encoding sequence of SEQ ID NO: 52;
f) a VH-encoding sequence of SEQ ID NO: 40 and a VL-encoding sequence of SEQ ID NO: 54;
g) a VH-encoding sequence of SEQ ID NO: 56 and a VL-encoding sequence of SEQ ID NO: 58;
h) a VH-encoding sequence of SEQ ID NO: 60 and a VL-encoding sequence of SEQ ID NO: 62;
i) a VH-encoding sequence of SEQ ID NO: 64 and a VL-encoding sequence of SEQ ID NO: 66;
j) a VH-encoding sequence of SEQ ID NO: 68 and a VL-encoding sequence of SEQ ID NO: 70; or
k) a VH-encoding sequence of SEQ ID NO: 72 and a VL-encoding sequence of SEQ ID NO: 74.
146. The synthetic polynucleotide of embodiment 144 or 145, wherein the polynucleotide comprises a sequence encoding an antigen-binding arm that binds CD3, said sequence comprising
a) a VH-encoding sequence of SEQ ID NO: 98 and a VL-encoding sequence of SEQ ID NO: 100;
b) a VH-encoding sequence of SEQ ID NO: 102 and a VL-encoding sequence of SEQ ID NO: 100;
c) a VH-encoding sequence of SEQ ID NO: 104 and a VL-encoding sequence of SEQ ID NO: 100;
d) a VH-encoding sequence of SEQ ID NO: 106 and a VL-encoding sequence of SEQ ID NO: 100; or
e) a VH-encoding sequence of SEQ ID NO: 108 and a VL-encoding sequence of SEQ ID NO: 110.
147. An synthetic polynucleotide encoding the bispecific antibody or bispecific binding fragment of any one of embodiments 136 to 143.
148. The synthetic polynucleotide of embodiment 147, wherein the polynucleotide comprises a sequence encoding an HC1 comprising the nucleotide sequence of SEQ ID NO: 173, 177, or 181.
149. The synthetic polynucleotide of any one of embodiments 147-148, wherein the polynucleotide comprises a sequence encoding a LC comprising the nucleotide sequence of SEQ ID NO: 175, 179, or 183.
150. The synthetic polynucleotide of any one of embodiments 147-149, comprising
a) an HC1-encoding sequence of SEQ ID NO: 173 and a LC-encoding sequence of SEQ ID NO: 175;
b) an HC1-encoding sequence of SEQ ID NO: 177 and a LC-encoding sequence of SEQ ID NO: 179; or
c) an HC1-encoding sequence of SEQ ID NO: 181 and a LC-encoding sequence of SEQ ID NO: 183.
151. The synthetic polynucleotide of any one of embodiments 147-150, wherein the polynucleotide comprises a sequence encoding a second antigen-binding arm polypeptide, said sequence comprising the nucleotide sequence of SEQ ID NO: 165 or 190.
152. The synthetic polynucleotide of any one of embodiments 147-151, comprising
a) an HC1-encoding sequence of SEQ ID NO: 173 and a LC-encoding sequence of SEQ ID NO: 175 and a sequence of SEQ ID NO: 165 encoding a second antigen-binding arm polypeptide;
b) an HC1-encoding sequence of SEQ ID NO: 177 and a LC-encoding sequence of SEQ ID NO: 179 and a sequence of SEQ ID NO: 165 encoding a second antigen-binding arm polypeptide;
c) an HC1-encoding sequence of SEQ ID NO: 181 and a LC-encoding sequence of SEQ ID NO: 183 and a sequence of SEQ ID NO: 165 encoding a second antigen-binding arm polypeptide;
d) an HC1-encoding sequence of SEQ ID NO: 173 and a LC-encoding sequence of SEQ ID NO: 175 and a sequence of SEQ ID NO: 190 encoding a second antigen-binding arm polypeptide;
e) an HC1-encoding sequence of SEQ ID NO: 177 and a LC-encoding sequence of SEQ ID NO: 179 and a sequence of SEQ ID NO: 190 encoding a second antigen-binding arm polypeptide; or
f) an HC1-encoding sequence of SEQ ID NO: 181 and a LC-encoding sequence of SEQ ID NO: 183 and a sequence of SEQ ID NO: 190 encoding a second antigen-binding arm polypeptide.
153. A pharmaceutical composition comprising the bispecific antibody or bispecific binding fragment of any one of embodiments 114 to 143, or the polynucleotide of any one of embodiments 144-152, and a pharmaceutically acceptable carrier.
154. The pharmaceutical composition of embodiment 153, wherein the pharmaceutical composition further comprises a second therapeutic agent.
155. The pharmaceutical composition of embodiment 154, wherein the second therapeutic agent comprises a chemotherapeutic agent, anti-CD20 agent, anti-CD19 agent, anti-CD22 agent, Bruton's tyrosine kinase (BTK) inhibitor, mucosa-associated lymphoid tissue lymphoma translocation protein 1 (MALT1) inhibitor, immunomodulatory imide drug (IMiD), pro apoptotic B cell lymphoma 2 (Bcl-2) family inhibitor, phosphoinositide 3-kinase (PI3K) inhibitor, immune checkpoint inhibitor, CD28 costimulatory bispecific antibody, CD137 costimulatory bispecific antibody, or a combination thereof.
156. An isolated cell expressing the bispecific antibody or bispecific binding fragment of any one of embodiments 114 to 143.
157. The cell of embodiment 156 wherein the cell is a hybridoma.
158. The cell of embodiment 156 wherein the antibody is recombinantly produced.
159. A method for treating cancer in a subject in need thereof, said method comprising administering to the subject a therapeutically effective amount of the bispecific antibody or bispecific binding fragment of any one of embodiments 114 to 143, the polynucleotide of any one of embodiments 144-152, or the pharmaceutical composition of any one of embodiments 153-155.
160. The method of embodiment 159, wherein the bispecific antibody or bispecific binding fragment or the pharmaceutical composition is administered for a time sufficient to treat the cancer.
161. A method for inhibiting growth or proliferation of a cancer cell, said method comprising administering to said cell an effective amount of the bispecific antibody or bispecific binding fragment of any one of embodiments 114 to 143, the polynucleotide of any one of embodiments 144-152, or the pharmaceutical composition of any one of embodiments 153-155, wherein said effective amount is sufficient to inhibit the growth or proliferation of said cancer cell.
162. The method of embodiment 161, wherein said cancer cell is in a subject and the bispecific antibody or bispecific binding fragment or the pharmaceutical composition is administered to the subject.
163. The method of embodiment 161, wherein said administration is conducted ex vivo.
164. A method of redirecting a T cell to CD79b-expressing cancer cells in a subject in need thereof, said method comprising administering to said subject a therapeutically effective amount of the bispecific antibody or bispecific binding fragment of any one of embodiments 114 to 143, the polynucleotide of any one of embodiments 144-152, or the pharmaceutical composition of any one of embodiments 153-155.
165. The method of embodiment 164, wherein the said therapeutically effective amount is sufficient to direct said T cell response to said cancer cells.
166. The method of any one of embodiments 159-165, wherein the cancer is a hematological cancer.
167. The method of embodiment 166 wherein the hematological cancer is a CD79b-expressing B cell cancer.
168. The method of embodiment 167 wherein the CD79b-expressing B cell cancer is a B-cell lymphoma or a non-Hodgkin lymphoma.
169. The method of embodiment 168 wherein the CD79b-expressing B cell cancer is a diffuse large B-cell lymphoma (DLBCL), mantle cell lymphoma (MCL), marginal zone lymphoma (MZL), follicular lymphoma (FL), chronic lymphocytic leukemia (CLL), or Waldenström macroglobulinemia (WM).
170. The method of any one of embodiments 159-169, wherein the cancer is relapsed, refractory, or malignant cancer, or any combination thereof.
171. The method of any one of embodiments 159-169 further comprising administering a second therapeutic agent.
172. The method of embodiment 171 wherein the second therapeutic agent is a surgery, chemotherapy, androgen deprivation therapy or radiation, anti-CD20 agent, anti-CD19 agent, anti-CD22 agent, Bruton's tyrosine kinase (BTK) inhibitor, mucosa-associated lymphoid tissue lymphoma translocation protein 1 (MALT1) inhibitor, immunomodulatory imide drug (IMiD), pro apoptotic B cell lymphoma 2 (Bcl-2) family inhibitor, phosphoinositide 3-kinase (PI3K) inhibitor, immune checkpoint inhibitor, CD28 costimulatory bispecific antibody, CD137 costimulatory bispecific antibody, or any combination thereof.
173. The method of any one of embodiments 159-160, 161 and 163-172, wherein the bispecific antibody or bispecific binding fragment, polynucleotide, or the pharmaceutical composition is administered intravenously, intramuscularly, intraperitoneally, and/or subcutaneously to the subject.
174. The method of any one of embodiments 159-160, 161 and 163-173, wherein the bispecific antibody or bispecific binding fragment, polynucleotide, or the pharmaceutical composition is administered subcutaneously to the subject.
175. A method for generating the bispecific antibody or bispecific binding fragment of any one of embodiments 114 to 143, wherein said method comprises culturing the cell of any one of embodiments 156-158 and isolating said bispecific antibody or bispecific binding fragment.
176. A kit comprising (i) the bispecific antibody or bispecific binding fragment of any one of embodiments 114 to 143 and/or a polynucleotide of any one of embodiments 144-152, and (ii) packaging for the same.
177. A trispecific antibody, or a trispecific binding fragment thereof, comprising:
a) a first heavy chain portion (HC1) comprising a first heavy chain variable domain (VH);
b) a light chain portion (LC) comprising a light chain variable domain (VL); and
c) a second heavy chain portion (HC2) comprising a second VH domain, wherein
(i) the HC1 VH and LC VL domains form a first antigen-binding domain that binds a first antigen,
(ii) the HC2 VH domain forms a second antigen-binding domain that binds a second antigen,
(iii) the HC1 or the HC2 further comprise a third VH domain forming a third antigen-binding domain that binds a third antigen,
(iv) the HC1 and HC2 each optionally comprise a Fragment crystallizable (Fc) domain comprising a CH2-CH3 domain; and wherein the first antigen is cluster of differentiation 79B protein (CD79b), and
(v) the second antigen is cluster of differentiation 3 (CD3), and the third antigen is cluster of differentiation 20 (CD20); or
(vi) the second antigen is cluster of differentiation 20 (CD20), and the third antigen is cluster of differentiation 3 (CD3).
178. The trispecific antibody or trispecific binding fragment of embodiment 177, wherein the HC2 comprises the third VH domain forming the third antigen-binding domain that binds the third antigen.
179. The trispecific antibody or trispecific binding fragment of embodiment 177 or 178, wherein the HC2 comprises, from N to C-terminus, the second VH domain forming the second antigen-binding domain, the Fc domain, a first linker (L1), and the third VH domain forming the third antigen-binding domain.
180. The trispecific antibody or trispecific binding fragment of any one of embodiments 177-179, wherein the HC2 comprises the second VH domain forming the the second antigen-binding domain that binds CD3, and the HC2 further comprises the third VH domain forming the third antigen-binding domain that binds CD20.
181. The trispecific antibody or trispecific binding fragment of any one of embodiments 177-180, wherein the HC1 VH and LC VL form an antigen-binding fragment (Fab) comprising the first antigen-binding domain.
182. The trispecific antibody or trispecific binding fragment of any one of embodiments 177-181, wherein the HC2 VH forms a single-chain variable fragment (scFv) comprising the second antigen-binding domain.
183. The trispecific antibody or trispecific binding fragment of any one of embodiments 177-182, wherein the third VH forms a single-chain variable fragment (scFv) comprising the third antigen-binding domain.
184. The trispecific antibody or trispecific binding fragment of any one of embodiments 177-183, wherein the Fc domains of HC1 and HC2 comprise one or more different mutations which promote heterodimerization.
185. The trispecific antibody or trispecific binding fragment of embodiment 184, wherein the Fc domain of the HC1 comprise mutations T366S, L368A and Y407V (EU numbering) and the Fc domain of the HC2 comprises mutation T366W (EU numbering).
186. The trispecific antibody or trispecific binding fragment of embodiment 184, wherein the Fc domain of the HC2 comprise mutations T366S, L368A and Y407V (EU numbering) and the Fc domain of the HC1 comprises mutation T366W (EU numbering).
187. The trispecific antibody or trispecific binding fragment of any one of embodiments 177-186, wherein the Fc domains of HC1 and/or HC2 further comprise one or more mutations which reduce Fc binding to a Fcγ receptor.
188. The trispecific antibody or trispecific binding fragment of embodiment 187, wherein the Fcγ receptor is FcγRI, FcγRIIA, FcγRIIB, FcγRIIIA, and/or FcγRIIIB.
189. The trispecific antibody or trispecific binding fragment of embodiment 187 or 188, wherein the Fc domains of HC1 and/or HC2 each comprise one or more mutations selected from L234A, L235A, and D265S (EU numbering).
190. The trispecific antibody or trispecific binding fragment of embodiment 189, wherein the Fc domains of HC1 and HC2 each comprise mutations L234A, L235A, and D265S (EU numbering).
191. The trispecific antibody or trispecific binding fragment of any one of embodiments 177-190, wherein the Fc domains of HC1 or HC2 further comprise one or more mutations which reduce Fc binding to protein A.
192. The trispecific antibody or trispecific binding fragment of embodiment 191, wherein the Fc domains of HC1 or HC2 comprise mutations H435R and/or Y436F (EU numbering).
193. The trispecific antibody or trispecific binding fragment of embodiment 192, wherein the Fc domain of HC1 comprises mutations H435R and Y436F (EU numbering).
194. The trispecific antibody or trispecific binding fragment of any one of embodiments 179-193, wherein the first linker (L1) comprises any one of the amino acid sequences of SEQ ID NOs: 215-248.
195. The trispecific antibody or trispecific binding fragment of any one of embodiments 179-194, wherein the first linker (L1) comprises the amino acid sequence of GGSEGKSSGSGSESKSTGGS (SEQ ID NO: 215), GGGGSGGGGS (SEQ ID NO: 248) or GGGGSGGGGSGGGGSGGGGS ((G₄S)₄, SEQ ID NO: 221).
196. An bispecific antibody, or a bispecific binding fragment thereof, comprising:
   a) a first heavy chain portion (HC1) comprising a first heavy chain variable domain (VH);
   b) a light chain portion (LC) comprising a light chain variable domain (VL); and
   c) a second heavy chain portion (HC2) comprising a second VH domain, wherein
(i) the HC1 VH and the LC VL domains form a first antigen-binding domain that binds a first antigen,
(ii) the HC2 VH domain forms a second antigen-binding domain that binds a second antigen,
(iii) the HC1 and HC2 each optionally comprise a Fragment crystallizable (Fc) domain comprising a CH2-CH3 domain; and
wherein the first antigen is cluster of differentiation 79B protein (CD79b), and the second antigen is cluster of differentiation 3 (CD3).
197. The bispecific antibody or bispecific binding fragment of any one of embodiments 196, wherein the HC1 VH and LC VL form an antigen-binding fragment (Fab) comprising the first antigen-binding domain.
198. The bispecific antibody or bispecific binding fragment of embodiment 196-197, wherein the HC2 VH forms a single-chain variable fragment (scFv) comprising the second antigen-binding domain.
199. The bispecific antibody or bispecific binding fragment of any one of embodiments 196-198, wherein the Fe domains of HC1 and HC2 comprise one or more different mutations which promote heterodimerization.
200. The bispecific antibody or bispecific binding fragment of embodiment 199, wherein the Fc domain of the HC1 comprise mutations T366S, L368A and Y407V (EU numbering) and the Fc domain of the HC2 comprises mutation T366W (EU numbering).
201. The bispecific antibody or bispecific binding fragment of embodiment 199, wherein the Fc domain of the HC2 comprise mutations T366S, L368A and Y407V (EU numbering) and the Fc domain of the HC1 comprises mutation T366W (EU numbering).
202. The bispecific antibody or bispecific binding fragment of any one of embodiments 196-201, wherein the Fc domains of HC1 and/or HC2 further comprise one or more mutations which reduce Fc binding to a Fcγ receptor.
203. The bispecific antibody or bispecific binding fragment of embodiment 202, wherein the Fcγ receptor is FcγRI, FcγRIIA, FcγRIIB, FcγRIIIA, and/or FcγRIIIB.
204. The bispecific antibody or bispecific binding fragment of embodiment 202 or 203, wherein the Fc domains of HC1 and/or HC2 each comprise one or more mutations selected from L234A, L235A, and D265S (EU numbering).
205. The bispecific antibody or bispecific binding fragment of embodiment 204, wherein the Fc domains of HC1 and HC2 each comprise mutations L234A, L235A, and D265S (EU numbering).
206. The bispecific antibody or bispecific binding fragment of any one of embodiments 196-205, wherein the Fc domains of HC1 or HC2 further comprise one or more mutations which reduce Fc binding to protein A.
207. The bispecific antibody or bispecific binding fragment of embodiment 206, wherein the Fc domains of HC1 or HC2 comprise mutations H435R and/or Y436F (EU numbering).
208. The bispecific antibody or bispecific binding fragment of embodiment 207, wherein the Fc domain of HC1 comprises mutations H435R and Y436F (EU numbering).
209. The bispecific antibody or bispecific binding fragment of any one of embodiments 196-208, wherein the first antigen-binding domain that binds CD79b comprises:
   a) a heavy chain complementarity determining region (HCDR) 1, a HCDR2 and a HCDR3 of a heavy chain variable domain (VH) of SEQ ID NO: 35 and a light chain complementarity determining region (LCDR) 1, a LCDR2 and a LCDR3 of a light chain variable domain (VL) of SEQ ID NO: 37;
   b) the HCDR1, the HCDR2 and the HCDR3 of the VH of SEQ ID NO: 39 and the LCDR1, the LCDR2 and the LCDR3 of the VL of SEQ ID NO: 41;
   c) the HCDR1, the HCDR2 and the HCDR3 of the VH of SEQ ID NO: 43 and the LCDR1, the LCDR2 and the LCDR3 of the VL of SEQ ID NO: 41;
   d) the HCDR1, the HCDR2 and the HCDR3 of the VH of SEQ ID NO: 45 and the LCDR1, the LCDR2 and the LCDR3 of the VL of SEQ ID NO: 47;
   e) the HCDR1, the HCDR2 and the HCDR3 of the VH of SEQ ID NO: 49 and the LCDR1, the LCDR2 and the LCDR3 of the VL of SEQ ID NO: 51;
   f) the HCDR1, the HCDR2 and the HCDR3 of the VH of SEQ ID NO: 39 and the LCDR1, the LCDR2 and the LCDR3 of the VL of SEQ ID NO: 53;
   g) the HCDR1, the HCDR2 and the HCDR3 of the VH of SEQ ID NO: 55 and the LCDR1, the LCDR2 and the LCDR3 of the VL of SEQ ID NO: 57;
   h) the HCDR1, the HCDR2 and the HCDR3 of the VH of SEQ ID NO: 59 and the LCDR1, the LCDR2 and the LCDR3 of the VL of SEQ ID NO: 61;
   i) the HCDR1, the HCDR2 and the HCDR3 of the VH of SEQ ID NO: 63 and the LCDR1, the LCDR2 and the LCDR3 of the VL of SEQ ID NO: 65;
   j) the HCDR1, the HCDR2 and the HCDR3 of the VH of SEQ ID NO: 67 and the LCDR1, the LCDR2 and the LCDR3 of the VL of SEQ ID NO: 69; or
   k) the HCDR1, the HCDR2 and the HCDR3 of the VH of SEQ ID NO: 71 and the LCDR1, the LCDR2 and the LCDR3 of the VL of SEQ ID NO: 73.
210. The bispecific antibody or bispecific binding fragment of any one of embodiments 196-209, wherein the first antigen-binding domain that binds CD79b comprises the HCDR1, the HCDR2, the HCDR3, the LCDR1, the LCDR2 and the LCDR3 of:
   a) SEQ ID NOs: 1, 2, 3, 4, 5 and 6, respectively;
   b) SEQ ID NOs: 13, 8, 9, 10, 11 and 12, respectively;
   c) SEQ ID NOs: 7, 8, 9, 10, 11 and 12, respectively;
   d) SEQ ID NOs: 14, 15, 16, 17, 5 and 6, respectively;
   e) SEQ ID NOs: 18, 8, 19, 20, 21 and 12, respectively;
   f) SEQ ID NOs: 22, 23, 24, 25, 5 and 6, respectively;
   g) SEQ ID NOs: 22, 26, 27, 28, 5 and 29, respectively; or
   h) SEQ ID NOs: 30, 31, 32, 33, 5 and 6, respectively.
211. The bispecific antibody or bispecific binding fragment of any one of embodiments 196-210, wherein the first antigen-binding domain that binds CD79b comprises:
   a) the VH of SEQ ID NO: 35 and the VL of SEQ ID NO: 37;
   b) the VH of SEQ ID NO: 39 and the VL of SEQ ID NO: 41;
   c) the VH of SEQ ID NO: 43 and the VL of SEQ ID NO: 41;
   d) the VH of SEQ ID NO: 45 and the VL of SEQ ID NO: 47;
   e) the VH of SEQ ID NO: 49 and the VL of SEQ ID NO: 51;
   f) the VH of SEQ ID NO: 39 and the VL of SEQ ID NO: 53;
   g) the VH of SEQ ID NO: 55 and the VL of SEQ ID NO: 57;
   h) the VH of SEQ ID NO: 59 and the VL of SEQ ID NO: 61;
   i) the VH of SEQ ID NO: 63 and the VL of SEQ ID NO: 65;
   j) the VH of SEQ ID NO: 67 and the VL of SEQ ID NO: 69; or
   k) the VH of SEQ ID NO: 71 and the VL of SEQ ID NO: 73.
212. The bispecific antibody or bispecific binding fragment of any one of embodiments 196-211, wherein the second antigen-binding domain that binds CD3 comprises:
   a) the HCDR1, the HCDR2 and the HCDR3 of the VH of SEQ ID NO: 97 and the LCDR1, the LCDR2 and the LCDR3 of the VL of SEQ ID NO: 99;
   b) the HCDR1, the HCDR2 and the HCDR3 of the VH of SEQ ID NO: 101 and the LCDR1, the LCDR2 and the LCDR3 of the VL of SEQ ID NO: 99;
   c) the HCDR1, the HCDR2 and the HCDR3 of the VH of SEQ ID NO: 103 and the LCDR1, the LCDR2 and the LCDR3 of the VL of SEQ ID NO: 99;
   d) the HCDR1, the HCDR2 and the HCDR3 of the VH of SEQ ID NO: 105 and the LCDR1, the LCDR2 and the LCDR3 of the VL of SEQ ID NO: 99; or
   e) the HCDR1, the HCDR2 and the HCDR3 of the VH of SEQ ID NO: 107 and the LCDR1, the LCDR2 and the LCDR3 of the VL of SEQ ID NO: 109.
213. The bispecific antibody or bispecific binding fragment of any one of embodiments 196-212, wherein the second antigen-binding domain that binds CD3 comprises the HCDR1, the HCDR2, the HCDR3, the LCDR1, the LCDR2 and the LCDR3 of:
   a) SEQ ID NOs: 76, 77, 78, 79, 80 and 81, respectively;
   b) SEQ ID NOs: 76, 77, 75, 79, 80 and 81, respectively;
   c) SEQ ID NOs: 76, 77, 82, 79, 80 and 81, respectively; or
   d) SEQ ID NOs: 83, 84, 85, 86, 87 and 88, respectively.
214. The bispecific antibody or bispecific binding fragment of any one of embodiments 196-213, wherein the second or third antigen-binding domain that binds CD3 comprises:
   a) the VH of SEQ ID NO: 97 and the VL of SEQ ID NO: 99;
   b) the VH of SEQ ID NO: 101 and the VL of SEQ ID NO: 99;
   c) the VH of SEQ ID NO: 103 and the VL of SEQ ID NO: 99;
   d) the VH of SEQ ID NO: 105 and the VL of SEQ ID NO: 99; or
   e) the VH of SEQ ID NO: 107 and the VL of SEQ ID NO: 109.
215. The bispecific antibody or bispecific binding fragment of any one of embodiments 198-214, wherein the scFv comprises, from the N- to C-terminus, a VH, a linker (L) and a VL (VH-L-VL) or the VL, the linker (L) and the VH (VL-L-VH).
216. The bispecific antibody or bispecific binding fragment of any one of embodiments 198-215, wherein the scFv comprises, from the N- to C-terminus, the VL, the linker (L) and the VH (VL-L-VH).
217. The bispecific antibody or bispecific binding fragment of embodiment 215 or 216, wherein the linker (L) comprises any one of amino acid sequence of SEQ ID NOs: 215-248.
218. The bispecific antibody or bispecific binding fragment of any one of embodiments 215-217, wherein the linker (L) comprises an amino acid sequence of GGSEGKSSGSGSESKSTGGS (SEQ ID NO: 215).
219. The bispecific antibody or bispecific binding fragment of any one of embodiments 196-218, wherein the first antigen-binding domain specifically binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
220. The bispecific antibody or bispecific binding fragment of embodiment 219, wherein the first antigen-binding domain specifically binds to CD79b with an affinity of about 1×10⁻¹¹-1×10⁻⁹ M.
221. The bispecific antibody or bispecific binding fragment of any one of embodiments 196-220, wherein the second antigen-binding domain specifically binds to residues 54-58 (GSEIL; SEQ ID NO: 257), residues 74-75 (NI), and/or residues 100-105 (PRGSKP; SEQ ID NO: 258) of human CD3ε.
222. The bispecific antibody or bispecific binding fragment of any one of embodiments 196-221, wherein the antibody or antigen-binding fragment thereof is an IgG1, IgG2, IgG3, or IgG4 isotype.
223. The bispecific antibody or bispecific binding fragment of any of embodiments 196-222, wherein the antibody or antigen-binding fragment thereof is an IgG1 isotype.
224. The bispecific antibody or bispecific binding fragment of embodiment 196, wherein the HC1 comprises the amino acid sequence of SEQ ID NO: 172, 176, or 180.
225. The bispecific antibody or bispecific binding fragment of embodiment 196 or 224, wherein the LC comprises the amino acid sequence of SEQ ID NO: 174, 178, or 182.
226. The bispecific antibody or bispecific binding fragment of any one of embodiments 196, 224-225, wherein
   a. the HC1 comprises the amino acid sequence of SEQ ID NO: 172 and the LC comprises the amino acid sequence of SEQ ID NO: 174;
   b. the HC1 comprises the amino acid sequence of SEQ ID NO: 176 and the LC comprises the amino acid sequence of SEQ ID NO: 178;
   c. the HC1 comprises the amino acid sequence of SEQ ID NO: 180 and the LC comprises the amino acid sequence of SEQ ID NO: 182.
227. The bispecific antibody or bispecific binding fragment of any one of embodiments 196, 224-226, wherein the HC2 comprises the amino acid sequence of SEQ ID NO: 164 or 189.
228. The bispecific antibody or bispecific binding fragment of any one of embodiments 196, 224-227, wherein
a. the HC1 comprises the amino acid sequence of SEQ ID NO: 172, the LC comprises the amino acid sequence of SEQ ID NO: 174, and the HC2 comprises the amino acid sequence of SEQ ID NO: 164;
b. the HC1 comprises the amino acid sequence of SEQ ID NO: 176, the LC comprises the amino acid sequence of SEQ ID NO: 178, and the HC2 comprises the amino acid sequence of SEQ ID NO: 164;
c. the HC1 comprises the amino acid sequence of SEQ ID NO: 180, the LC comprises the amino acid sequence of SEQ ID NO: 182, and the HC2 comprises the amino acid sequence of SEQ ID NO: 164;
d. the HC1 comprises the amino acid sequence of SEQ ID NO: 172, the LC comprises the amino acid sequence of SEQ ID NO: 174, and the HC2 comprises the amino acid sequence of SEQ ID NO: 189;
e. the HC1 comprises the amino acid sequence of SEQ ID NO: 176, the LC comprises the amino acid sequence of SEQ ID NO: 178, and the HC2 comprises the amino acid sequence of SEQ ID NO: 189; or
f. the HC1 comprises the amino acid sequence of SEQ ID NO: 180, the LC comprises the amino acid sequence of SEQ ID NO: 182, and the HC2 comprises the amino acid sequence of SEQ ID NO: 189.

The invention can also be defined by the following numbered clauses.
1. A trispecific antibody, or a trispecific binding fragment thereof, comprising:
   a) a first heavy chain (HC1);
   b) a light chain (LC); and
   c) a second heavy chain (HC2),
   wherein
   (i) the HC1 and the LC form a first antigen-binding site that specifically binds a first antigen,
   (ii) the HC2 comprises a second antigen-binding site that specifically binds a second antigen,
   (iii) the HC1 or the HC2 further comprises a third antigen-binding site that specifically binds a third antigen,
   (iv) the HC1 and HC2 each comprise a Fragment crystallizable (Fc) domain comprising a CH2-CH3 domain; and wherein the first antigen is cluster of differentiation 79B protein (CD79b), and
   (v) the second antigen is cluster of differentiation 3 (CD3), and the third antigen is cluster of differentiation 20 (CD20); or
   (vi) the second antigen is cluster of differentiation 20 (CD20), and the third antigen is cluster of differentiation 3 (CD3).
2. The trispecific antibody or trispecific binding fragment of clause 1, wherein the HC2 comprises the third antigen-binding site that specifically binds the third antigen.
3. The trispecific antibody or trispecific binding fragment of clause 2, wherein the HC2 comprises, from N to C-terminus, the second antigen-binding site, the Fc domain, a first linker (L1), and the third antigen-binding site.
4. The trispecific antibody or trispecific binding fragment of any one of clauses 1-3, wherein the HC2 comprises the second antigen-binding site that specifically binds CD3, and the HC2 further comprises the third antigen-binding site that specifically binds CD20.
5. The trispecific antibody or trispecific binding fragment of any one of clauses 1-4, wherein the first antigen-binding site comprises an antigen-binding fragment (Fab).
6. The trispecific antibody or trispecific binding fragment of any one of clauses 1-5, wherein the second antigen-binding site comprises a single-chain variable fragment (scFv).
7. The trispecific antibody or trispecific binding fragment of any one of clauses 1-6, wherein the third antigen-binding site comprises a single-chain variable fragment (scFv).
8. The trispecific antibody or trispecific binding fragment of any one of clauses 1-7, wherein the Fc domains of HC1 and HC2 comprise one or more different mutations which promote heterodimerization.
9. The trispecific antibody or trispecific binding fragment of clause 8, wherein the Fc domain of the HC1 comprise mutations T366S, L368A and Y407V (EU numbering) and the Fc domain of the HC2 comprises mutation T366W (EU numbering).
10. The trispecific antibody or trispecific binding fragment of clause 8, wherein the Fc domain of the HC2 comprise mutations T366S, L368A and Y407V (EU numbering) and the Fc domain of the HC1 comprises mutation T366W (EU numbering).
11. The trispecific antibody or trispecific binding fragment of any one of clauses 1-10, wherein the Fc domains of HC1 and/or HC2 further comprise one or more mutations which reduce Fc binding to a Fcγ receptor.
12. The trispecific antibody or trispecific binding fragment of clause 11, wherein the Fcγ receptor is FcγRI, FcγRIIA, FcγRIIB, FcγRIIIA, and/or FcγRIIIB.
13. The trispecific antibody or trispecific binding fragment of clause 11 or 12, wherein the Fc domains of HC1 and/or HC2 each comprise one or more mutations selected from L234A, L235A, and D265S (EU numbering).
14. The trispecific antibody or trispecific binding fragment of clause 13, wherein the Fc domains of HC1 and HC2 each comprise mutations L234A, L235A, and D265S (EU numbering).
15. The trispecific antibody or trispecific binding fragment of any one of clauses 1-14, wherein the Fc domains of HC1 or HC2 further comprises one or more mutations which reduce Fc binding to protein A.
16. The trispecific antibody or trispecific binding fragment of clause 15, wherein the Fc domains of HC1 or HC2 comprises mutations H435R and/or Y436F (EU numbering).
17. The trispecific antibody or trispecific binding fragment of clause 16, wherein the Fc domain of HC1 comprises mutations H435R and Y436F (EU numbering).
18. The trispecific antibody or trispecific binding fragment of any one of clauses 3-17, wherein the first linker (L1) comprises any one of amino acid sequence of SEQ ID NOs: 215-248.
19. The trispecific antibody or trispecific binding fragment of any one of clauses 3-18, wherein the first linker (L1) comprises the amino acid sequence of GGSEGKSSGSGSESKSTGGS (SEQ ID NO: 215), GGGGSGGGGS (SEQ ID NO: 248) or GGGGSGGGGSGGGGSGGGGS ((G₄S)₄, SEQ ID NO: 221).
20. The trispecific antibody or trispecific binding fragment of any one of clauses 1-19, wherein the first antigen-binding site that specifically binds CD79b comprises:
   a) a heavy chain complementarity determining region (HCDR) 1, a HCDR2 and a HCDR3 of a heavy chain variable region (VH) of SEQ ID NO: 35 and a light chain complementarity determining region (LCDR) 1, a LCDR2 and a LCDR3 of a light chain variable region (VL) of SEQ ID NO: 37;
   b) the HCDR1, the HCDR2 and the HCDR3 of the VH of SEQ ID NO: 39 and the LCDR1, the LCDR2 and the LCDR3 of the VL of SEQ ID NO: 41;
   c) the HCDR1, the HCDR2 and the HCDR3 of the VH of SEQ ID NO: 43 and the LCDR1, the LCDR2 and the LCDR3 of the VL of SEQ ID NO: 41;
   d) the HCDR1, the HCDR2 and the HCDR3 of the VH of SEQ ID NO: 45 and the LCDR1, the LCDR2 and the LCDR3 of the VL of SEQ ID NO: 47;
   e) the HCDR1, the HCDR2 and the HCDR3 of the VH of SEQ ID NO: 49 and the LCDR1, the LCDR2 and the LCDR3 of the VL of SEQ ID NO: 51;
   f) the HCDR1, the HCDR2 and the HCDR3 of the VH of SEQ ID NO: 39 and the LCDR1, the LCDR2 and the LCDR3 of the VL of SEQ ID NO: 53;
   g) the HCDR1, the HCDR2 and the HCDR3 of the VH of SEQ ID NO: 55 and the LCDR1, the LCDR2 and the LCDR3 of the VL of SEQ ID NO: 57;
   h) the HCDR1, the HCDR2 and the HCDR3 of the VH of SEQ ID NO: 59 and the LCDR1, the LCDR2 and the LCDR3 of the VL of SEQ ID NO: 61;
   i) the HCDR1, the HCDR2 and the HCDR3 of the VH of SEQ ID NO: 63 and the LCDR1, the LCDR2 and the LCDR3 of the VL of SEQ ID NO: 65;
   j) the HCDR1, the HCDR2 and the HCDR3 of the VH of SEQ ID NO: 67 and the LCDR1, the LCDR2 and the LCDR3 of the VL of SEQ ID NO: 69; or
   k) the HCDR1, the HCDR2 and the HCDR3 of the VH of SEQ ID NO: 71 and the LCDR1, the LCDR2 and the LCDR3 of the VL of SEQ ID NO: 73.
21. The trispecific antibody or trispecific binding fragment of any one of clauses 1-20, wherein the first antigen-binding site that specifically binds CD79b comprises the HCDR1, the HCDR2, the HCDR3, the LCDR1, the LCDR2 and the LCDR3 of:
   a) SEQ ID NOs: 1, 2, 3, 4, 5 and 6, respectively;
   b) SEQ ID NOs: 13, 8, 9, 10, 11 and 12, respectively;
   c) SEQ ID NOs: 7, 8, 9, 10, 11 and 12, respectively;
   d) SEQ ID NOs: 14, 15, 16, 17, 5 and 6, respectively;
   e) SEQ ID NOs: 18, 8, 19, 20, 21 and 12, respectively;
   f) SEQ ID NOs: 22, 23, 24, 25, 5 and 6, respectively;
   g) SEQ ID NOs: 22, 26, 27, 28, 5 and 29, respectively; or
   h) SEQ ID NOs: 30, 31, 32, 33, 5 and 6, respectively.
22. The trispecific antibody or trispecific binding fragment of any one of clauses 1-21, wherein the first antigen-binding site that specifically binds CD79b comprises:
   a) the VH of SEQ ID NO: 35 and the VL of SEQ ID NO: 37;
   b) the VH of SEQ ID NO: 39 and the VL of SEQ ID NO: 41;
   c) the VH of SEQ ID NO: 43 and the VL of SEQ ID NO: 41;
   d) the VH of SEQ ID NO: 45 and the VL of SEQ ID NO: 47;
   e) the VH of SEQ ID NO: 49 and the VL of SEQ ID NO: 51;
   f) the VH of SEQ ID NO: 39 and the VL of SEQ ID NO: 53;
   g) the VH of SEQ ID NO: 55 and the VL of SEQ ID NO: 57;
   h) the VH of SEQ ID NO: 59 and the VL of SEQ ID NO: 61;
   i) the VH of SEQ ID NO: 63 and the VL of SEQ ID NO: 65;
   j) the VH of SEQ ID NO: 67 and the VL of SEQ ID NO: 69; or
   k) the VH of SEQ ID NO: 71 and the VL of SEQ ID NO: 73.
23. The trispecific antibody or trispecific binding fragment of any one of clauses 1-22, wherein the second or third antigen-binding site that specifically binds CD3 comprises:
   a) the HCDR1, the HCDR2 and the HCDR3 of the VH of SEQ ID NO: 97 and the LCDR1, the LCDR2 and the LCDR3 of the VL of SEQ ID NO: 99;
   b) the HCDR1, the HCDR2 and the HCDR3 of the VH of SEQ ID NO: 101 and the LCDR1, the LCDR2 and the LCDR3 of the VL of SEQ ID NO: 99;
   c) the HCDR1, the HCDR2 and the HCDR3 of the VH of SEQ ID NO: 103 and the LCDR1, the LCDR2 and the LCDR3 of the VL of SEQ ID NO: 99;
   d) the HCDR1, the HCDR2 and the HCDR3 of the VH of SEQ ID NO: 105 and the LCDR1, the LCDR2 and the LCDR3 of the VL of SEQ ID NO: 99; or
   e) the HCDR1, the HCDR2 and the HCDR3 of the VH of SEQ ID NO: 107 and the LCDR1, the LCDR2 and the LCDR3 of the VL of SEQ ID NO: 109.
24. The trispecific antibody or trispecific binding fragment of any one of clauses 1-23, wherein the second or third antigen-binding site that specifically binds CD3 comprises the HCDR1, the HCDR2, the HCDR3, the LCDR1, the LCDR2 and the LCDR3 of:
   a) SEQ ID NOs: 76, 77, 78, 79, 80 and 81, respectively;
   b) SEQ ID NOs: 76, 77, 75, 79, 80 and 81, respectively;
   c) SEQ ID NOs: 76, 77, 82, 79, 80 and 81, respectively; or
   d) SEQ ID NOs: 83, 84, 85, 86, 87 and 88, respectively.
25. The trispecific antibody or trispecific binding fragment of any one of clauses 1-24, wherein the second or third antigen-binding site that specifically binds CD3 comprises:
   a) the VH of SEQ ID NO: 97 and the VL of SEQ ID NO: 99;
   b) the VH of SEQ ID NO: 101 and the VL of SEQ ID NO: 99;
   c) the VH of SEQ ID NO: 103 and the VL of SEQ ID NO: 99;
   d) the VH of SEQ ID NO: 105 and the VL of SEQ ID NO: 99; or
   e) the VH of SEQ ID NO: 107 and the VL of SEQ ID NO: 109.
26. The trispecific antibody or trispecific binding fragment of any one of clauses 1-25, wherein the second or third antigen-binding site that specifically binds CD20 comprises:
   a) the HCDR1, the HCDR2 and the HCDR3 of the VH of SEQ ID NO: 126 and the LCDR1, the LCDR2 and the LCDR3 of the VL of SEQ ID NO: 128;
   b) the HCDR1, the HCDR2 and the HCDR3 of the VH of SEQ ID NO: 130 and the LCDR1, the LCDR2 and the LCDR3 of the VL of SEQ ID NO: 132;
   c) the HCDR1, the HCDR2 and the HCDR3 of the VH of SEQ ID NO: 134 and the LCDR1, the LCDR2 and the LCDR3 of the VL of SEQ ID NO: 136; or
   d) the HCDR1, the HCDR2 and the HCDR3 of the VH of SEQ ID NO: 138 and the LCDR1, the LCDR2 and the LCDR3 of the VL of SEQ ID NO: 140.
27. The trispecific antibody or trispecific binding fragment of any one of clauses 1-26, wherein the second or third antigen-binding site that specifically binds CD20 comprises the HCDR1, the HCDR2, the HCDR3, the LCDR1, the LCDR2 and the LCDR3 of:
   a) SEQ ID NOs: 115, 116, 117, 118, 119 and 120, respectively;
   b) SEQ ID NOs: 121, 122, 123, 124, 119 and 125, respectively;
   c) SEQ ID NOs: 115, 116, 95, 96, 119 and 125, respectively; or
   d) SEQ ID NOs: 121, 116, 123, 124, 119 and 125, respectively.
28. The trispecific antibody or trispecific binding fragment of any one of clauses 1-27, wherein the second or third antigen-binding site that specifically binds CD20 comprises:
   a) the VH of SEQ ID NO: 126 and the VL of SEQ ID NO: 128;
   b) the VH of SEQ ID NO: 130 and the VL of SEQ ID NO: 132;
   c) the VH of SEQ ID NO: 134 and the VL of SEQ ID NO: 136; or
   d) the VH of SEQ ID NO: 138 and the VL of SEQ ID NO: 140.
29. The trispecific antibody or trispecific binding fragment of any one of clauses 1-19, wherein the first antigen-binding site that specifically binds CD79b comprises the HCDR1, the HCDR2 and the HCDR3 of the VH of SEQ ID NO: 35 and the LCDR1, the LCDR2 and the LCDR3 of the VL of SEQ ID NO: 37;
   the second antigen-binding site that specifically binds CD3 comprises the HCDR1, the HCDR2 and the HCDR3 of the VH of SEQ ID NO: 107 and the LCDR1, the LCDR2 and the LCDR3 of the VL of SEQ ID NO: 109; and
   the third antigen-binding site that specifically binds CD20 comprises the HCDR1, the HCDR2 and the HCDR3 of the VH of SEQ ID NO: 130 and the LCDR1, the LCDR2 and the LCDR3 of the VL of SEQ ID NO: 132.
30. The trispecific antibody or trispecific binding fragment of any one of clauses 1-19 and 29, wherein the first antigen-binding site that specifically binds CD79b comprises the HCDR1, the HCDR2, the HCDR3, the LCDR1, the LCDR2 and the LCDR3 of SEQ ID NOs: 1, 2, 3, 4, 5 and 6, respectively;
   the second antigen-binding site that specifically binds CD3 comprises the HCDR1, the HCDR2, the HCDR3, the LCDR1, the LCDR2 and the LCDR3 of SEQ ID NOs: 83, 84, 85, 86, 87 and 88, respectively; and
   the third antigen-binding site that specifically binds CD20 comprises the HCDR1, the HCDR2, the HCDR3, the LCDR1, the LCDR2 and the LCDR3 of SEQ ID NOs: 121, 122, 123, 124, 119 and 125, respectively.
31. The trispecific antibody or trispecific binding fragment of any one of clauses 1-19, 29 and 30, wherein the first antigen-binding site that specifically binds CD79b comprises the VH of SEQ ID NO: 35 and the VL of SEQ ID NO: 37;
   the second antigen-binding site that specifically binds CD3 comprises the VH of SEQ ID NO: 107 and the VL of SEQ ID NO: 109; and
   the third antigen-binding site that specifically binds CD20 comprises the VH of SEQ ID NO: 130 and the VL of SEQ ID NO: 132.
32. The trispecific antibody or trispecific binding fragment of any one of clauses 1-19, wherein the first antigen-binding site that specifically binds CD79b comprises the HCDR1, the HCDR2 and the HCDR3 of the VH of SEQ ID NO: 35 and the LCDR1, the LCDR2 and the LCDR3 of the VL of SEQ ID NO: 37;
   the second antigen-binding site that specifically binds CD3 comprises the HCDR1, the HCDR2 and the HCDR3 of the VH of SEQ ID NO: 101 and the LCDR1, the LCDR2 and the LCDR3 of the VL of SEQ ID NO: 99; and
   the third antigen-binding site that specifically binds CD20 comprises the HCDR1, the HCDR2 and the HCDR3 of the VH of SEQ ID NO: 130 and the LCDR1, the LCDR2 and the LCDR3 of the VL of SEQ ID NO: 132.
33. The trispecific antibody or trispecific binding fragment of any one of clauses 1-19 and 32, wherein the first antigen-binding site that specifically binds CD79b comprises the HCDR1, the HCDR2, the HCDR3, the LCDR1, the LCDR2 and the LCDR3 of SEQ ID NOs: 1, 2, 3, 4, 5 and 6, respectively;
   The second antigen-binding site that specifically binds CD3 comprises the HCDR1, the HCDR2, the HCDR3, the LCDR1, the LCDR2 and the LCDR3 of SEQ ID NOs: 76, 77, 75, 79, 80 and 81, respectively; and
   the third antigen-binding site that specifically binds CD20 comprises the HCDR1, the HCDR2, the HCDR3, the LCDR1, the LCDR2 and the LCDR3 of SEQ ID NOs: 121, 122, 123, 124, 119 and 125, respectively.
34. The trispecific antibody or trispecific binding fragment of any one of clauses 1-19, 32, and 33, wherein the first antigen-binding site that specifically binds CD79b comprises the VH of SEQ ID NO: 35 and the VL of SEQ ID NO: 37;
   the second antigen-binding site that specifically binds CD3 comprises the VH of SEQ ID NO: 101 and the VL of SEQ ID NO: 99; and
   the third antigen-binding site that specifically binds CD20 comprises the VH of SEQ ID NO: 130 and the VL of SEQ ID NO: 132.
35. The trispecific antibody or trispecific binding fragment of any one of clauses 6-34, wherein the scFv comprises, from the N- to C-terminus, a VH, a second linker (L2) and a VL (VH-L2-VL) or the VL, the L2 and the VH (VL-L2-VH).
36. The trispecific antibody or trispecific binding fragment of any one of clauses 6-35, wherein the scFv comprises, from the N- to C-terminus, the VL, the L2 and the VH (VL-L2-VH).
37. The trispecific antibody or trispecific binding fragment of clause 35 or 36, wherein the second linker (L2) comprises any one of amino acid sequence of SEQ ID NOs: 215-248.
38. The trispecific antibody or trispecific binding fragment of any one of clauses 35-37, wherein the L2 comprises an amino acid sequence of GGSEGKSSGSGSESKSTGGS (SEQ ID NO: 215).
39. The trispecific antibody or trispecific antigen-binding fragment of any one of clauses 1 to 38, wherein the first antigen-binding site specifically binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
40. The trispecific antibody or trispecific antigen-binding fragment of clause 39, wherein the first antigen-binding site specifically binds to CD79b with an affinity of about 1×10⁻¹¹-1×10⁻⁹ M.
41. The trispecific antibody or trispecific antigen-binding fragment of any one of clauses 1 to 40, wherein the antibody or antigen-binding fragment thereof are of IgG1, IgG2, IgG3, or IgG4 isotype.
42. The trispecific antibody or trispecific antigen-binding fragment of any of clauses 1 to 41 is an IgG1 isotype.
43. The trispecific antibody or trispecific binding fragment of clause 1, wherein the HC1 comprises the amino acid sequence of SEQ ID NO: 172, 176, 180, or 191.
44. The trispecific antibody or trispecific binding fragment of clause 1 or 43, wherein the LC comprises the amino acid sequence of SEQ ID NO: 174, 178 or 182.
45. The trispecific antibody or trispecific binding fragment of any one of clauses 1, 43 and 44, wherein
   a) the HC1 comprises the amino acid sequence of SEQ ID NO: 172 and the LC comprises the amino acid sequence of SEQ ID NO: 174;
   b) the HC1 comprises the amino acid sequence of SEQ ID NO: 176 and the LC comprises the amino acid sequence of SEQ ID NO: 178;
   c) the HC1 comprises the amino acid sequence of SEQ ID NO: 180 and the LC comprises the amino acid sequence of SEQ ID NO: 182;
   d) the HC1 comprises the amino acid sequence of SEQ ID NO: 191 and the LC comprises the amino acid sequence of SEQ ID NO: 182.
46. The trispecific antibody or trispecific binding fragment of any one of clauses 1, 43-45, wherein the HC2 comprises the amino acid sequence of SEQ ID NO: 142, 144, 148, 150, 152, 154, 156, 158, 160, 162, 166, 168, or 170.
47. The trispecific antibody or trispecific binding fragment of clause 1, 43-46, wherein
   a) the HC1 comprises the amino acid sequence of SEQ ID NO: 172, the LC comprises the amino acid sequence of SEQ ID NO: 174, and the HC2 comprises the amino acid sequence of SEQ ID NO: 142;
   b) the HC1 comprises the amino acid sequence of SEQ ID NO: 176, the LC comprises the amino acid sequence of SEQ ID NO: 178, and the HC2 comprises the amino acid sequence of SEQ ID NO: 142;
   c) the HC1 comprises the amino acid sequence of SEQ ID NO: 180, the LC comprises the amino acid sequence of SEQ ID NO: 182, and the HC2 comprises the amino acid sequence of SEQ ID NO: 142;
   d) the HC1 comprises the amino acid sequence of SEQ ID NO: 172, the LC comprises the amino acid sequence of SEQ ID NO: 174, and the HC2 comprises the amino acid sequence of SEQ ID NO: 144;
   e) the HC1 comprises the amino acid sequence of SEQ ID NO: 176, the LC comprises the amino acid sequence of SEQ ID NO: 178, and the HC2 comprises the amino acid sequence of SEQ ID NO: 144;
   f) the HC1 comprises the amino acid sequence of SEQ ID NO: 180, the LC comprises the amino acid sequence of SEQ ID NO: 182, and the HC2 comprises the amino acid sequence of SEQ ID NO: 144;
   g) the HC1 comprises the amino acid sequence of SEQ ID NO: 180, the LC comprises the amino acid sequence of SEQ ID NO: 182, and the HC2 comprises the amino acid sequence of SEQ ID NO: 148;
   h) the HC1 comprises the amino acid sequence of SEQ ID NO: 180, the LC comprises the amino acid sequence of SEQ ID NO: 182, and the HC2 comprises the amino acid sequence of SEQ ID NO: 150;
   i) the HC1 comprises the amino acid sequence of SEQ ID NO: 180, the LC comprises the amino acid sequence of SEQ ID NO: 182, and the HC2 comprises the amino acid sequence of SEQ ID NO: 152;
   j) the HC1 comprises the amino acid sequence of SEQ ID NO: 180, the LC comprises the amino acid sequence of SEQ ID NO: 182, and the HC2 comprises the amino acid sequence of SEQ ID NO: 154;
   k) the HC1 comprises the amino acid sequence of SEQ ID NO: 180, the LC comprises the amino acid sequence of SEQ ID NO: 182, and the HC2 comprises the amino acid sequence of SEQ ID NO: 156;
   l) the HC1 comprises the amino acid sequence of SEQ ID NO: 180, the LC comprises the amino acid sequence of SEQ ID NO: 182, and the HC2 comprises the amino acid sequence of SEQ ID NO: 158;
   m) the HC1 comprises the amino acid sequence of SEQ ID NO: 180, the LC comprises the amino acid sequence of SEQ ID NO: 182, and the HC2 comprises the amino acid sequence of SEQ ID NO: 160;
   n) the HC1 comprises the amino acid sequence of SEQ ID NO: 180, the LC comprises the amino acid sequence of SEQ ID NO: 182, and the HC2 comprises the amino acid sequence of SEQ ID NO: 162;
   o) the HC1 comprises the amino acid sequence of SEQ ID NO: 191, the LC comprises the amino acid sequence of SEQ ID NO: 182, and the HC2 comprises the amino acid sequence of SEQ ID NO: 166;
   p) the HC1 comprises the amino acid sequence of SEQ ID NO: 172, the LC comprises the amino acid sequence of SEQ ID NO: 174, and the HC2 comprises the amino acid sequence of SEQ ID NO: 168; or
   q) the HC1 comprises the amino acid sequence of SEQ ID NO: 172, the LC comprises the amino acid sequence of SEQ ID NO: 174, and the HC2 comprises the amino acid sequence of SEQ ID NO: 170.
48. The trispecific antibody or trispecific binding fragment of any one of clause 1, 43-47, wherein the HC1 comprises the amino acid sequence of SEQ ID NO: 172, the LC comprises the amino acid sequence of SEQ ID NO: 174, and the HC2 comprises the amino acid sequence of SEQ ID NO: 168.
49. The trispecific antibody or trispecific binding fragment of clause 1, 43-47, wherein the HC1 comprises the amino acid sequence of SEQ ID NO: 172, the LC comprises the amino acid sequence of SEQ ID NO: 174, and the HC2 comprises the amino acid sequence of SEQ ID NO: 170.
50. A trispecific antibody, or a trispecific binding fragment thereof, comprising:
   a) a first antigen-binding site that specifically binds CD79b,
   b) a second antigen-binding site that specifically binds CD3, and
   c) a third antigen-binding site that specifically binds CD20.
51. The trispecific antibody or trispecific binding fragment of clause 50, wherein the first antigen-binding site that specifically binds CD79b comprises:
   a) the HCDR1, the HCDR2 and the HCDR3 of the VH of SEQ ID NO: 35 and the LCDR1, the LCDR2 and the LCDR3 of the VL of SEQ ID NO: 37;
   b) the HCDR1, the HCDR2 and the HCDR3 of the VH of SEQ ID NO: 39 and the LCDR1, the LCDR2 and the LCDR3 of the VL of SEQ ID NO: 41;
   c) the HCDR1, the HCDR2 and the HCDR3 of the VH of SEQ ID NO: 43 and the LCDR1, the LCDR2 and the LCDR3 of the VL of SEQ ID NO: 41;
   d) the HCDR1, the HCDR2 and the HCDR3 of the VH of SEQ ID NO: 45 and the LCDR1, the LCDR2 and the LCDR3 of the VL of SEQ ID NO: 47;
   e) the HCDR1, the HCDR2 and the HCDR3 of the VH of SEQ ID NO: 49 and the LCDR1, the LCDR2 and the LCDR3 of the VL of SEQ ID NO: 51;
   f) the HCDR1, the HCDR2 and the HCDR3 of the VH of SEQ ID NO: 39 and the LCDR1, the LCDR2 and the LCDR3 of the VL of SEQ ID NO: 53;
   g) the HCDR1, the HCDR2 and the HCDR3 of the VH of SEQ ID NO: 55 and the LCDR1, the LCDR2 and the LCDR3 of the VL of SEQ ID NO: 57;
   h) the HCDR1, the HCDR2 and the HCDR3 of the VH of SEQ ID NO: 59 and the LCDR1, the LCDR2 and the LCDR3 of the VL of SEQ ID NO: 61;
   i) the HCDR1, the HCDR2 and the HCDR3 of the VH of SEQ ID NO: 63 and the LCDR1, the LCDR2 and the LCDR3 of the VL of SEQ ID NO: 65;
   j) the HCDR1, the HCDR2 and the HCDR3 of the VH of SEQ ID NO: 67 and the LCDR1, the LCDR2 and the LCDR3 of the VL of SEQ ID NO: 69; or
   k) the HCDR1, the HCDR2 and the HCDR3 of the VH of SEQ ID NO: 71 and the LCDR1, the LCDR2 and the LCDR3 of the VL of SEQ ID NO: 73.
52. The trispecific antibody or trispecific binding fragment of clause 50 or 51, wherein the first antigen-binding site that specifically binds CD79b comprises the HCDR1, the HCDR2, the HCDR3, the LCDR1, the LCDR2 and the LCDR3 of:
   a) SEQ ID NOs: 1, 2, 3, 4, 5 and 6, respectively;
   b) SEQ ID NOs: 13, 8, 9, 10, 11 and 12, respectively;
   c) SEQ ID NOs: 7, 8, 9, 10, 11 and 12, respectively;
   d) SEQ ID NOs: 14, 15, 16, 17, 5 and 6, respectively;
   e) SEQ ID NOs: 18, 8, 19, 20, 21 and 12, respectively;
   f) SEQ ID NOs: 22, 23, 24, 25, 5 and 6, respectively;
   g) SEQ ID NOs: 22, 26, 27, 28, 5 and 29, respectively; or
   h) SEQ ID NOs: 30, 31, 32, 33, 5 and 6, respectively.
53. The trispecific antibody or trispecific binding fragment of any one of clauses 50-52, wherein the first antigen-binding site that specifically binds CD79b comprises
   a) the VH of SEQ ID NO: 35 and the VL of SEQ ID NO: 37;
   b) the VH of SEQ ID NO: 39 and the VL of SEQ ID NO: 41;
   c) the VH of SEQ ID NO: 43 and the VL of SEQ ID NO: 41;
   d) the VH of SEQ ID NO: 45 and the VL of SEQ ID NO: 47;
   e) the VH of SEQ ID NO: 49 and the VL of SEQ ID NO: 51;
   f) the VH of SEQ ID NO: 39 and the VL of SEQ ID NO: 53;
   g) the VH of SEQ ID NO: 55 and the VL of SEQ ID NO: 57;
   h) the VH of SEQ ID NO: 59 and the VL of SEQ ID NO: 61;
   i) the VH of SEQ ID NO: 63 and the VL of SEQ ID NO: 65;
   j) the VH of SEQ ID NO: 67 and the VL of SEQ ID NO: 69; or
   k) the VH of SEQ ID NO: 71 and the VL of SEQ ID NO: 73.
54. The trispecific antibody or trispecific binding fragment of any one of clauses 50-53, wherein the second antigen-binding site that specifically binds CD3 comprises:
   a) the HCDR1, the HCDR2 and the HCDR3 of the VH of SEQ ID NO: 97 and the LCDR1, the LCDR2 and the LCDR3 of the VL of SEQ ID NO: 99;
   b) the HCDR1, the HCDR2 and the HCDR3 of the VH of SEQ ID NO: 101 and the LCDR1, the LCDR2 and the LCDR3 of the VL of SEQ ID NO: 99;
   c) the HCDR1, the HCDR2 and the HCDR3 of the VH of SEQ ID NO: 103 and the LCDR1, the LCDR2 and the LCDR3 of the VL of SEQ ID NO: 99;
   d) the HCDR1, the HCDR2 and the HCDR3 of the VH of SEQ ID NO: 105 and the LCDR1, the LCDR2 and the LCDR3 of the VL of SEQ ID NO: 99; or
   e) the HCDR1, the HCDR2 and the HCDR3 of the VH of SEQ ID NO: 107 and the LCDR1, the LCDR2 and the LCDR3 of the VL of SEQ ID NO: 109.
55. The trispecific antibody or trispecific binding fragment of any one of clauses 50-54, wherein the second antigen-binding site that specifically binds CD3 comprises:
   a) SEQ ID NOs: 76, 77, 78, 79, 80 and 81, respectively;
   b) SEQ ID NOs: 76, 77, 75, 79, 80 and 81, respectively;
   c) SEQ ID NOs: 76, 77, 82, 79, 80 and 81, respectively; or
   d) SEQ ID NOs: 83, 84, 85, 86, 87 and 88, respectively.
56. The trispecific antibody or trispecific binding fragment of any one of clauses 50-55, wherein the second antigen-binding site that specifically binds CD3 comprises
   a) the VH of SEQ ID NO: 97 and the VL of SEQ ID NO: 99;
   b) the VH of SEQ ID NO: 101 and the VL of SEQ ID NO: 99;
   c) the VH of SEQ ID NO: 103 and the VL of SEQ ID NO: 99;
   d) the VH of SEQ ID NO: 105 and the VL of SEQ ID NO: 99; or
   e) the VH of SEQ ID NO: 107 and the VL of SEQ ID NO: 109.
57. The trispecific antibody or trispecific binding fragment of any one of clauses 50-56, wherein the third antigen-binding site that specifically binds CD20 comprises:
   a) the HCDR1, the HCDR2 and the HCDR3 of the VH of SEQ ID NO: 126 and the LCDR1, the LCDR2 and the LCDR3 of the VL of SEQ ID NO: 128;
   b) the HCDR1, the HCDR2 and the HCDR3 of the VH of SEQ ID NO: 130 and the LCDR1, the LCDR2 and the LCDR3 of the VL of SEQ ID NO: 132;
   c) the HCDR1, the HCDR2 and the HCDR3 of the VH of SEQ ID NO: 134 and the LCDR1, the LCDR2 and the LCDR3 of the VL of SEQ ID NO: 136;
   d) the HCDR1, the HCDR2 and the HCDR3 of the VH of SEQ ID NO: 138 and the LCDR1, the LCDR2 and the LCDR3 of the VL of SEQ ID NO: 140.
58. The trispecific antibody or trispecific binding fragment of any one of clauses 50-57, wherein the third antigen-binding site that specifically binds CD20 comprises:
   a) SEQ ID NOs: 115, 116, 117, 118, 119 and 120, respectively;
   b) SEQ ID NOs: 121, 122, 123, 124, 119 and 125, respectively;
   c) SEQ ID NOs: 115, 116, 95, 96, 119 and 125, respectively; or
   d) SEQ ID NOs: 121, 116, 123, 124, 119 and 125, respectively.
59. The trispecific antibody or trispecific binding fragment of any one of clauses 50-58, wherein the third antigen-binding site that specifically binds CD20 comprises
   a) the VH of SEQ ID NO: 126 and the VL of SEQ ID NO: 128;
   b) the VH of SEQ ID NO: 130 and the VL of SEQ ID NO: 132;
   c) the VH of SEQ ID NO: 134 and the VL of SEQ ID NO: 136; or
   d) the VH of SEQ ID NO: 138 and the VL of SEQ ID NO: 140.
60. The trispecific antibody or trispecific binding fragment of any one of clauses 50-59, comprising a first antigen-binding site that specifically binds CD79b comprising
   the HCDR1, the HCDR2 and the HCDR3 of the VH of SEQ ID NO: 35 and the LCDR1, the LCDR2 and the LCDR3 of the VL of SEQ ID NO: 37;
   a second antigen-binding site that specifically binds CD3 comprising the HCDR1, the HCDR2 and the HCDR3 of the VH of SEQ ID NO: 107 and the LCDR1, the LCDR2 and the LCDR3 of the VL of SEQ ID NO: 109; and
   a third antigen-binding site that specifically binds CD20 comprising the HCDR1, the HCDR2 and the HCDR3 of the VH of SEQ ID NO: 130 and the LCDR1, the LCDR2 and the LCDR3 of the VL of SEQ ID NO: 132.
61. The trispecific antibody or trispecific binding fragment of any one of clauses 50-60, comprising a first antigen-binding site that specifically binds CD79b comprising
   the HCDR1, the HCDR2, the HCDR3, the LCDR1, the LCDR2 and the LCDR3 of SEQ ID NOs: 1, 2, 3, 4, 5 and 6, respectively;
   a second antigen-binding site that specifically binds CD3 comprising the HCDR1, the HCDR2, the HCDR3, the LCDR1, the LCDR2 and the LCDR3 of SEQ ID NOs: 83, 84, 85, 86, 87 and 88, respectively; and
   a third antigen-binding site that specifically binds CD20 comprising the HCDR1, the HCDR2, the HCDR3, the LCDR1, the LCDR2 and the LCDR3 of SEQ ID NOs: 121, 122, 123, 124, 119 and 125, respectively.
62. The trispecific antibody or trispecific binding fragment of any one of clauses 50-61, comprising a first antigen-binding site that specifically binds CD79b comprising
   the VH of SEQ ID NO: 35 and the VL of SEQ ID NO: 37;
   a second antigen-binding site that specifically binds CD3 comprising the VH of SEQ ID NO: 107 and the VL of SEQ ID NO: 109; and
   a third antigen-binding site that specifically binds CD20 comprising the VH of SEQ ID NO: 130 and the VL of SEQ ID NO: 132.
63. The trispecific antibody or trispecific binding fragment of any one of clauses 50-59, comprising a first antigen-binding site that specifically binds CD79b comprising the HCDR1, the HCDR2 and the HCDR3 of the VH of SEQ ID NO: 35 and the LCDR1, the LCDR2 and the LCDR3 of the VL of SEQ ID NO: 37;
   a second antigen-binding site that specifically binds CD3 comprising the HCDR1, the HCDR2 and the HCDR3 of the VH of SEQ ID NO: 101 and the LCDR1, the LCDR2 and the LCDR3 of the VL of SEQ ID NO: 99; and
   a third antigen-binding site that specifically binds CD20 comprising the HCDR1, the HCDR2 and the HCDR3 of the VH of SEQ ID NO: 130 and the LCDR1, the LCDR2 and the LCDR3 of the VL of SEQ ID NO: 132.
64. The trispecific antibody or trispecific binding fragment of any one of clauses 50-59, and 63, comprising a first antigen-binding site that specifically binds CD79b comprising the HCDR1, the HCDR2, the HCDR3, the LCDR1, the LCDR2 and the LCDR3 of SEQ ID NOs: 1, 2, 3, 4, 5 and 6, respectively;
   a second antigen-binding site that specifically binds CD3 comprising the HCDR1, the HCDR2, the HCDR3, the LCDR1, the LCDR2 and the LCDR3 of SEQ ID NOs: 76, 77, 75, 79, 80 and 81, respectively; and
   a third antigen-binding site that specifically binds CD20 comprising the HCDR1, the HCDR2, the HCDR3, the LCDR1, the LCDR2 and the LCDR3 of SEQ ID NOs: 121, 122, 123, 124, 119 and 125, respectively.
65. The trispecific antibody or trispecific binding fragment of any one of clauses 50-59, 63 and 64, comprising a first antigen-binding site that specifically binds CD79b comprising the VH of SEQ ID NO: 35 and the VL of SEQ ID NO: 37;
   a second antigen-binding site that specifically binds CD3 comprising the VH of SEQ ID NO: 101 and the VL of SEQ ID NO: 99; and
   a third antigen-binding site that specifically binds CD20 comprising the VH of SEQ ID NO: 130 and the VL of SEQ ID NO: 132.
66. The antibody or antigen-binding fragment of any one of clauses 50-65, wherein the first antigen-binding site specifically binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
67. The antibody or antigen-binding fragment of clause 66, wherein the first antigen-binding site specifically binds to CD79b with an affinity of about 1×10⁻¹¹-1×10⁻⁹ M.
68. The antibody or antigen-binding fragment of any one of clauses 50-67, wherein the antibody or antigen-binding fragment thereof are of IgG1, IgG2, IgG3, or IgG4 isotype.
69. The antibody or antigen-binding fragment of any of clauses 50-68 is an IgG1 isotype.
70. A trispecific antibody, or a trispecific binding fragment thereof, comprising:
   a) a first heavy chain (HC1);
   b) a light chain (LC); and
   c) a second heavy chain (HC2),
   wherein HC1 comprises the amino acid sequence of SEQ ID NO: 172, LC comprises the amino acid sequence of SEQ ID NO: 174, and the HC2 comprises the amino acid sequence of SEQ ID NO: 168.
71. A trispecific antibody, or a trispecific binding fragment thereof, comprising:
   a) a first heavy chain (HC1);
   b) a light chain (LC); and
   c) a second heavy chain (HC2),
   wherein HC1 comprises the amino acid sequence of SEQ ID NO: 172, LC comprises the amino acid sequence of SEQ ID NO: 174, and the HC2 comprises the amino acid sequence of SEQ ID NO: 170.
72. A synthetic polynucleotide encoding the trispecific antibody or trispecific binding fragment of any one of clauses 1 to 71.
73. The synthetic polynucleotide of clause 72, wherein the polynucleotide comprises a sequence encoding an antigen-binding site that specifically binds CD79b, said sequence comprising
   a) a VH-encoding sequence of SEQ ID NO: 36 and a VL-encoding sequence of SEQ ID NO: 38 or 213;
   b) a VH-encoding sequence of SEQ ID NO: 40 and a VL-encoding sequence of SEQ ID NO: 42;
   c) a VH-encoding sequence of SEQ ID NO: 44 and a VL-encoding sequence of SEQ ID NO: 34;
   d) a VH-encoding sequence of SEQ ID NO: 46 and a VL-encoding sequence of SEQ ID NO: 48 or 214;
   e) a VH-encoding sequence of SEQ ID NO: 50 and a VL-encoding sequence of SEQ ID NO: 52;
   f) a VH-encoding sequence of SEQ ID NO: 40 and a VL-encoding sequence of SEQ ID NO: 54;
   g) a VH-encoding sequence of SEQ ID NO: 56 and a VL-encoding sequence of SEQ ID NO: 58;
   h) a VH-encoding sequence of SEQ ID NO: 60 and a VL-encoding sequence of SEQ ID NO: 62;
   i) a VH-encoding sequence of SEQ ID NO: 64 and a VL-encoding sequence of SEQ ID NO: 66;
   j) a VH-encoding sequence of SEQ ID NO: 68 and a VL-encoding sequence of SEQ ID NO: 70; or
   k) a VH-encoding sequence of SEQ ID NO: 72 and a VL-encoding sequence of SEQ ID NO: 74.
74. The synthetic polynucleotide of clause 72 or 73, wherein the polynucleotide comprises a sequence encoding an antigen-binding site that specifically binds CD3, said sequence comprising
   a) a VH-encoding sequence of SEQ ID NO: 98 and a VL-encoding sequence of SEQ ID NO: 100;
   b) a VH-encoding sequence of SEQ ID NO: 102 and a VL-encoding sequence of SEQ ID NO: 100;
   c) a VH-encoding sequence of SEQ ID NO: 104 and a VL-encoding sequence of SEQ ID NO: 100;
   d) a VH-encoding sequence of SEQ ID NO: 106 and a VL-encoding sequence of SEQ ID NO: 100; or
   e) a VH-encoding sequence of SEQ ID NO: 108 and a VL-encoding sequence of SEQ ID NO: 110.
75. The synthetic polynucleotide of any one of clauses 72-74, wherein the polynucleotide comprises a sequence encoding an antigen-binding site that specifically binds CD20, said sequence comprising:
   a) a VH-encoding sequence of SEQ ID NO: 127 and a VL-encoding sequence of SEQ ID NO: 129;
   b) a VH-encoding sequence of SEQ ID NO: 131 and a VL-encoding sequence of SEQ ID NO: 133;
   c) a VH-encoding sequence of SEQ ID NO: 135 and a VL-encoding sequence of SEQ ID NO: 137; or
   d) a VH-encoding sequence of SEQ ID NO: 139 and a VL-encoding sequence of SEQ ID NO: 141.
76. The synthetic polynucleotide of any one of clauses 72-75, wherein the polynucleotide comprises a sequence encoding an HC1 comprising the nucleotide sequence of SEQ ID NO: 173, 177, 181, or 192.
77. The synthetic polynucleotide of any one of clauses 72-76, wherein the polynucleotide comprises a sequence encoding a LC comprising the nucleotide sequence of SEQ ID NO: 175, 179, 183 or 188.
78. The synthetic polynucleotide of clause any one of clauses 72-77, comprising
   a) an HC1-encoding sequence of SEQ ID NO: 173 and a LC-encoding sequence of SEQ ID NO: 175;
   b) an HC1-encoding sequence of SEQ ID NO: 177, and a LC-encoding sequence of SEQ ID NO: 179;
   c) an HC1-encoding sequence of SEQ ID NO: 181, and a LC-encoding sequence of SEQ ID NO: 183;
   d) an HC1-encoding sequence of SEQ ID NO: 181, and a LC-encoding sequence of SEQ ID NO: 188;
   e) an HC1-encoding sequence of SEQ ID NO: 192, and a LC-encoding sequence of SEQ ID NO: 183.
79. The synthetic polynucleotide of any one of clauses 72-78, wherein the polynucleotide comprises a sequence encoding an HC2 comprising the nucleotide sequence of SEQ ID NO: 143, 145, 149, 151, 153, 155, 157, 159, 161, 163, 167, 169, or 171.
80. The synthetic polynucleotide of any one of clauses 72-79, comprising
   a) an HC1-encoding sequence of SEQ ID NO: 173, a LC-encoding sequence of SEQ ID NO: 175, and an HC2-encoding sequence of SEQ ID NO: 143;
   b) an HC1-encoding sequence of SEQ ID NO: 177, a LC-encoding sequence of SEQ ID NO: 179, and an HC2-encoding sequence of SEQ ID NO: 143;
   c) an HC1-encoding sequence of SEQ ID NO: 181, a LC-encoding sequence of SEQ ID NO: 183, and an HC2-encoding sequence of SEQ ID NO: 143;
   d) an HC1-encoding sequence of SEQ ID NO: 173, a LC-encoding sequence of SEQ ID NO: 175, and an HC2-encoding sequence of SEQ ID NO: 145;
   e) an HC1-encoding sequence of SEQ ID NO: 177, a LC-encoding sequence of SEQ ID NO: 179, and an HC2-encoding sequence of SEQ ID NO: 145;
   f) an HC1-encoding sequence of SEQ ID NO: 181, a LC-encoding sequence of SEQ ID NO: 183, and an HC2-encoding sequence of SEQ ID NO: 145;
   g) an HC1-encoding sequence of SEQ ID NO: 181, a LC-encoding sequence of SEQ ID NO: 188, and an HC2-encoding sequence of SEQ ID NO: 149;
   h) an HC1-encoding sequence of SEQ ID NO: 181, a LC-encoding sequence of SEQ ID NO: 188, and an HC2-encoding sequence of SEQ ID NO: 151;
   i) an HC1-encoding sequence of SEQ ID NO: 181, a LC-encoding sequence of SEQ ID NO: 188, and an HC2-encoding sequence of SEQ ID NO: 153;
   j) an HC1-encoding sequence of SEQ ID NO: 181, a LC-encoding sequence of SEQ ID NO: 188, and an HC2-encoding sequence of SEQ ID NO: 155;
   k) an HC1-encoding sequence of SEQ ID NO: 181, a LC-encoding sequence of SEQ ID NO: 188, and an HC2-encoding sequence of SEQ ID NO: 157;
   l) an HC1-encoding sequence of SEQ ID NO: 181, a LC-encoding sequence of SEQ ID NO: 188, and an HC2-encoding sequence of SEQ ID NO: 159;
   m) an HC1-encoding sequence of SEQ ID NO: 181, a LC-encoding sequence of SEQ ID NO: 188, and an HC2-encoding sequence of SEQ ID NO: 161;
   n) an HC1-encoding sequence of SEQ ID NO: 181, a LC-encoding sequence of SEQ ID NO: 188, and an HC2-encoding sequence of SEQ ID NO: 163;
   o) an HC1-encoding sequence of SEQ ID NO: 192, a LC-encoding sequence of SEQ ID NO: 183, and an HC2-encoding sequence of SEQ ID NO: 167;
   p) an HC1-encoding sequence of SEQ ID NO: 173, a LC-encoding sequence of SEQ ID NO: 175, and an HC2-encoding sequence of SEQ ID NO: 169; or
   q) an HC1-encoding sequence of SEQ ID NO: 173, a LC-encoding sequence of SEQ ID NO: 175, and an HC2-encoding sequence of SEQ ID NO: 171.
81. The synthetic polynucleotide of any one of clauses 72-80, comprising an HC1-encoding sequence of SEQ ID NO: 173, a LC-encoding sequence of SEQ ID NO: 175, and an HC2-encoding sequence of SEQ ID NO: 169.
82. The synthetic polynucleotide of any one of clauses 72-80, comprising an HC1-encoding sequence of SEQ ID NO: 173, a LC-encoding sequence of SEQ ID NO: 175, and an HC2-encoding sequence of SEQ ID NO: 171.
83. A synthetic polynucleotide encoding a trispecific antibody, or a trispecific binding fragment thereof, said trispecific antibody or trispecific binding fragment comprising:
   a) a first heavy chain (HC1);
   b) a light chain (LC); and
   c) a second heavy chain (HC2),
   wherein the polynucleotide comprises an HC1-encoding sequence of SEQ ID NO: 173, a LC-encoding sequence of SEQ ID NO: 175, and an HC2-encoding sequence of SEQ ID NO: 169.
84. A synthetic polynucleotide encoding a trispecific antibody, or a trispecific binding fragment thereof, said trispecific antibody or trispecific binding fragment comprising:
   a) a first heavy chain (HC1);
   b) a light chain (LC); and
   c) a second heavy chain (HC2),
   wherein the polynucleotide comprises an HC1-encoding sequence of SEQ ID NO: 173, a LC-encoding sequence of SEQ ID NO: 175, and an HC2-encoding sequence of SEQ ID NO: 171.
85. A pharmaceutical composition comprising the trispecific antibody or trispecific binding fragment of any one of clauses 1 to 71, or the polynucleotide of any one of clauses 72-84, and a pharmaceutically acceptable carrier.
86. An isolated cell expressing the trispecific antibody or trispecific binding fragment of any one of clauses 1 to 71.
87. The cell of clause 86 wherein the cell is a hybridoma.
88. The cell of clause 86 wherein the antibody is recombinantly produced.
89. A method for treating cancer in a subject in need thereof, said method comprising administering to the subject a therapeutically effective amount of the trispecific antibody or trispecific binding fragment of any one of clauses 1 to 71, the polynucleotide of any one of clauses 72-84, or the pharmaceutical composition of clause 85.
90. The method of clause 89, wherein the trispecific antibody or trispecific binding fragment, polynucleotide, or the pharmaceutical composition is administered for a time sufficient to treat the cancer.
91. A method for inhibiting growth or proliferation of a cancer cell, said method comprising administering to said cell an effective amount of the trispecific antibody or trispecific binding fragment of any one of clauses 1 to 71, the polynucleotide of any one of clauses 72-84, or the pharmaceutical composition of clause 85, wherein said effective amount is sufficient to inhibit the growth or proliferation of said cancer cell.
92. The method of clause 91, wherein said cancer cell is in a subject and the trispecific antibody or trispecific binding fragment, polynucleotide, or the pharmaceutical composition is administered to the subject.
93. The method of clause 92, wherein said administration is conducted ex vivo.
94. A method of redirecting a T cell to CD79b and/or CD20-expressing cancer cells in a subject in need thereof, said method comprising administering to said subject a therapeutically effective amount of the trispecific antibody or trispecific binding fragment of any one of clauses 1 to 71, the polynucleotide of any one of clauses 72-84, or the pharmaceutical composition of clause 85.
95. The method of clause 94, wherein the said therapeutically effective amount is sufficient to direct said T cell response to said cancer cells.
96. The method of any one of clauses 89-95, wherein the cancer is a hematological cancer.
97. The method of clause 96 wherein the hematological cancer is a CD79b and/or CD20-expressing B cell cancer.
98. The method of clause 97 wherein the CD79b and/or CD20-expressing B cell cancer is a B-cell lymphoma or a non-Hodgkin lymphoma.
99. The method of clause 98 wherein the CD79b and/or CD20-expressing B cell cancer is a diffuse large B-cell lymphoma (DLBCL), mantle cell lymphoma (MCL), marginal zone lymphoma (MZL), or follicular lymphoma (FL).
100. The method of any one of clauses 89-99, wherein the cancer is relapsed, refractory, or malignant cancer, or any combination thereof.
101. The method of any one of clauses 89-100 further comprising administering a second therapeutic agent.
102. The method of clause 101 wherein the second therapeutic agent is a surgery, chemotherapy, androgen deprivation therapy or radiation, or any combination thereof.
103. A method for generating the trispecific antibody or trispecific binding fragment of any one of clauses 1 to 71, wherein said method comprises culturing the cell of any one of clauses 86 to 88 and isolating said trispecific antibody or trispecific binding fragment.
104. A kit comprising (i) the trispecific antibody or trispecific binding fragment of any one of clauses 1 to 71 and/or the polynucleotide of any one of clauses 72-84 and (ii) packaging for the same.
105. An antibody, or an antigen-binding fragment thereof, that binds specifically to Cluster of Differentiation 79B protein (CD79b), comprising:
   a) a heavy chain complementarity determining region (HCDR) 1, a HCDR2 and a HCDR3 of a heavy chain variable region (VH) of SEQ ID NO: 35 and a light chain complementarity determining region (LCDR) 1, a LCDR2 and a LCDR3 of a light chain variable region (VL) of SEQ ID NO: 37;
   b) the HCDR1, the HCDR2 and the HCDR3 of the VH of SEQ ID NO: 39 and the LCDR1, the LCDR2 and the LCDR3 of the VL of SEQ ID NO: 41;
   c) the HCDR1, the HCDR2 and the HCDR3 of the VH of SEQ ID NO: 43 and the LCDR1, the LCDR2 and the LCDR3 of the VL of SEQ ID NO: 41;
   d) the HCDR1, the HCDR2 and the HCDR3 of the VH of SEQ ID NO: 45 and the LCDR1, the LCDR2 and the LCDR3 of the VL of SEQ ID NO: 47;
   e) the HCDR1, the HCDR2 and the HCDR3 of the VH of SEQ ID NO: 49 and the LCDR1, the LCDR2 and the LCDR3 of the VL of SEQ ID NO: 51;
   f) the HCDR1, the HCDR2 and the HCDR3 of the VH of SEQ ID NO: 39 and the LCDR1, the LCDR2 and the LCDR3 of the VL of SEQ ID NO: 53;
   g) the HCDR1, the HCDR2 and the HCDR3 of the VH of SEQ ID NO: 55 and the LCDR1, the LCDR2 and the LCDR3 of the VL of SEQ ID NO: 57;
   h) the HCDR1, the HCDR2 and the HCDR3 of the VH of SEQ ID NO: 59 and the LCDR1, the LCDR2 and the LCDR3 of the VL of SEQ ID NO: 61;
   i) the HCDR1, the HCDR2 and the HCDR3 of the VH of SEQ ID NO: 63 and the LCDR1, the LCDR2 and the LCDR3 of the VL of SEQ ID NO: 65;
   j) the HCDR1, the HCDR2 and the HCDR3 of the VH of SEQ ID NO: 67 and the LCDR1, the LCDR2 and the LCDR3 of the VL of SEQ ID NO: 69; or
   k) the HCDR1, the HCDR2 and the HCDR3 of the VH of SEQ ID NO: 71 and the LCDR1, the LCDR2 and the LCDR3 of the VL of SEQ ID NO: 73.
106. The antibody or antigen-binding fragment of clause 105, comprising the HCDR1, the HCDR2, the HCDR3, the LCDR1, the LCDR2 and the LCDR3 of:
   a) SEQ ID NOs: 1, 2, 3, 4, 5 and 6, respectively;
   b) SEQ ID NOs: 13, 8, 9, 10, 11 and 12, respectively;
   c) SEQ ID NOs: 7, 8, 9, 10, 11 and 12, respectively;
   d) SEQ ID NOs: 14, 15, 16, 17, 5 and 6, respectively;
   e) SEQ ID NOs: 18, 8, 19, 20, 21 and 12, respectively;
   f) SEQ ID NOs: 22, 23, 24, 25, 5 and 6, respectively;
   g) SEQ ID NOs: 22, 26, 27, 28, 5 and 29, respectively; or
   h) SEQ ID NOs: 30, 31, 32, 33, 5 and 6, respectively.
107. The antibody or antigen-binding fragment of clause 105 or 106, comprising
   a) the VH of SEQ ID NO: 35 and the VL of SEQ ID NO: 37;
   b) the VH of SEQ ID NO: 39 and the VL of SEQ ID NO: 41;
   c) the VH of SEQ ID NO: 43 and the VL of SEQ ID NO: 41;
   d) the VH of SEQ ID NO: 45 and the VL of SEQ ID NO: 47;
   e) the VH of SEQ ID NO: 49 and the VL of SEQ ID NO: 51;
   f) the VH of SEQ ID NO: 39 and the VL of SEQ ID NO: 53;
   g) the VH of SEQ ID NO: 55 and the VL of SEQ ID NO: 57;
   h) the VH of SEQ ID NO: 59 and the VL of SEQ ID NO: 61;
   i) the VH of SEQ ID NO: 63 and the VL of SEQ ID NO: 65;
   j) the VH of SEQ ID NO: 67 and the VL of SEQ ID NO: 69; or
   k) the VH of SEQ ID NO: 71 and the VL of SEQ ID NO: 73.
108. The antibody or antigen-binding fragment of any one of clauses 105-107, wherein the antibody or antigen-binding fragment specifically binds residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
109. The antibody or antigen-binding fragment of clause 108, wherein the antibody or antigen-binding fragment specifically binds to CD79b with an affinity of about 1×10⁻¹¹-1×10⁻⁹ M.
110. The antibody or antigen-binding fragment of any one of clauses 105-109 wherein the antibody or antigen-binding fragment is a human antibody or antigen-binding fragment.
111. The antibody or antigen-binding fragment of any one of clauses 105 to 110 wherein the antibody or antigen-binding fragment is recombinant.
112. The antigen binding fragment of any one of clauses 105 to 111 wherein the antigen binding fragment is a Fab fragment, a Fab2 fragment, a scFv, a (scFv)2, a Fv, a Fd, a dAb or a VHH.
113. The antibody or antigen-binding fragment of any one of clauses 105 to 112 wherein the antibody or antigen-binding fragment thereof are of IgG1, IgG2, IgG3, or IgG4 isotype.
114. The antibody or antigen-binding fragment of any of clauses 105 to 113 is an IgG1 or an IgG4 isotype.
115. A synthetic polynucleotide encoding the antibody or antigen-binding fragment of any one of clauses 105 to 114.
116. The synthetic polynucleotide of clause 115, comprising
   a) a VH-encoding sequence of SEQ ID NO: 36 and a VL-encoding sequence of SEQ ID NO: 38 or 213;
   b) a VH-encoding sequence of SEQ ID NO: 40 and a VL-encoding sequence of SEQ ID NO: 42;
   c) a VH-encoding sequence of SEQ ID NO: 44 and a VL-encoding sequence of SEQ ID NO: 34;
   d) a VH-encoding sequence of SEQ ID NO: 46 and a VL-encoding sequence of SEQ ID NO: 48 or 214;
   e) a VH-encoding sequence of SEQ ID NO: 50 and a VL-encoding sequence of SEQ ID NO: 52;
   f) a VH-encoding sequence of SEQ ID NO: 40 and a VL-encoding sequence of SEQ ID NO: 54;
   g) a VH-encoding sequence of SEQ ID NO: 56 and a VL-encoding sequence of SEQ ID NO: 58;
   h) a VH-encoding sequence of SEQ ID NO: 60 and a VL-encoding sequence of SEQ ID NO: 62;
   i) a VH-encoding sequence of SEQ ID NO: 64 and a VL-encoding sequence of SEQ ID NO: 66;
   j) a VH-encoding sequence of SEQ ID NO: 68 and a VL-encoding sequence of SEQ ID NO: 70; or
   k) a VH-encoding sequence of SEQ ID NO: 72 and a VL-encoding sequence of SEQ ID NO: 74.
117. A pharmaceutical composition comprising the antibody or antigen-binding fragment of any one of clauses 105 to 114, or the polynucleotide of clause 115 or 116, and a pharmaceutically acceptable carrier.
118. An isolated cell expressing the antibody or antigen-binding fragment of any one of clauses 105 to 114.
119. The cell of clause 118 wherein the cell is a hybridoma.
120. The cell of clause 118 wherein the antibody is recombinantly produced.
121. A method for treating cancer in a subject in need thereof, said method comprising administering to the subject a therapeutically effective amount of the antibody or antigen-binding fragment of any one of clauses 105 to 114 or the polynucleotide of clause 115 or 116, or the pharmaceutical composition of clause 117.
122. The method of clause 121, wherein the antibody or antigen-binding fragment or the pharmaceutical composition is administered for a time sufficient to treat the cancer.
123. A method for inhibiting growth or proliferation of a cancer cell, said method comprising administering to said cell an effective amount of the antibody or antigen-binding fragment of any one of clauses 105 to 114, or the polynucleotide of clause 115 or 116, or the pharmaceutical composition of clause 117, wherein said effective amount is sufficient to inhibit the growth or proliferation of said cancer cell.
124. The method of clause 123, wherein said cancer cell is in a subject and the antibody or antigen-binding fragment or the pharmaceutical composition is administered to the subject.
125. The method of clause 124, wherein said administration is conducted ex vivo.
126. The method of any one of clauses 121-125 wherein the cancer is a hematological cancer.
127. The method of clause 126 wherein the hematological cancer is a CD79b-expressing B cell cancer.
128. The method of clause 127 wherein the CD79b-expressing B cell cancer is a B-cell lymphoma or a non-Hodgkin lymphoma.
129. The method of clause 128 wherein the CD79b-expressing B cell cancer is a diffuse large B-cell lymphoma (DLBCL), mantle cell lymphoma (MCL), marginal zone lymphoma (MZL), or follicular lymphoma (FL).
130. The method of any one of clauses 121-125, wherein the cancer is relapsed, refractory, or malignant cancer, or any combination thereof.
131. The method of any one of clauses 121-130 further comprising administering a second therapeutic agent.
132. The method of clause 131 wherein the second therapeutic agent is a surgery, chemotherapy, androgen deprivation therapy or radiation, or any combination thereof.
133. A method for generating the antibody or antigen-binding fragment of any one of clauses 105 to 114, wherein said method comprises culturing the cell of any one of clauses 118 to 120 and isolating said antibody or antigen-binding fragment.
134. A kit comprising (i) the antibody or antigen-binding fragment of any one of clauses 105 to 114 and/or a polynucleotide of clause 115 or 116 and (ii) packaging for the same.
135. An bispecific antibody, or a bispecific binding fragment thereof, comprising:
   a) a first heavy chain (HC1);
   b) a light chain (LC); and
   c) a second heavy chain (HC2),
   wherein
   (i) the HC1 and the LC form a first antigen-binding site that specifically binds a first antigen,
   (ii) the HC2 comprises a second antigen-binding site that specifically binds a second antigen,
   (iii) the HC1 and HC2 each comprise a Fragment crystallizable (Fc) domain comprising a CH2-CH3 domain; and
   wherein the first antigen is cluster of differentiation 79B protein (CD79b), and the second antigen is cluster of differentiation 3 (CD3).
136. The bispecific antibody or bispecific binding fragment of any one of clauses 135, wherein the first antigen-binding site comprises an antigen-binding fragment (Fab).
137. The bispecific antibody or bispecific binding fragment of clause 135-136, wherein the second antigen-binding site comprises a single-chain variable fragment (scFv).
138. The bispecific antibody or bispecific binding fragment of any one of clauses 135-137, wherein the Fc domains of HC1 and HC2 comprise one or more different mutations which promote heterodimerization.
139. The bispecific antibody or bispecific binding fragment of clause 138, wherein the Fc domain of the HC1 comprise mutations T366S, L368A and Y407V (EU numbering) and the Fc domain of the HC2 comprises mutation T366W (EU numbering).
140. The bispecific antibody or bispecific binding fragment of clause 138, wherein the Fc domain of the HC2 comprise mutations T366S, L368A and Y407V (EU numbering) and the Fc domain of the HC1 comprises mutation T366W (EU numbering).
141. The bispecific antibody or bispecific binding fragment of any one of clauses 135-140, wherein the Fc domains of HC1 and/or HC2 further comprise one or more mutations which reduce Fc binding to a Fcγ receptor.
142. The bispecific antibody or bispecific binding fragment of clause 141, wherein the Fcγ receptor is FcγRI, FcγRIIA, FcγRIIB, FcγRIIIA, and/or FcγRIIIB.
143. The bispecific antibody or bispecific binding fragment of clause 141 or 142, wherein the Fc domains of HC1 and/or HC2 each comprise one or more mutations selected from L234A, L235A, and D265S (EU numbering).
144. The bispecific antibody or bispecific binding fragment of clause 143, wherein the Fc domains of HC1 and HC2 each comprise mutations L234A, L235A, and D265S (EU numbering).
145. The bispecific antibody or bispecific binding fragment of any one of clauses 135-144, wherein the Fc domains of HC1 or HC2 further comprises one or more mutations which reduce Fc binding to protein A.
146. The bispecific antibody or bispecific binding fragment of clause 145, wherein the Fc domains of HC1 or HC2 comprises mutations H435R and/or Y436F (EU numbering).
147. The bispecific antibody or bispecific binding fragment of clause 146, wherein the Fc domain of HC1 comprises mutations H435R and Y436F (EU numbering).
148. The bispecific antibody or bispecific binding fragment of any one of clauses 135-147, wherein the first antigen-binding site that specifically binds CD79b comprises:
   a) a heavy chain complementarity determining region (HCDR) 1, a HCDR2 and a HCDR3 of a heavy chain variable region (VH) of SEQ ID NO: 35 and a light chain complementarity determining region (LCDR) 1, a LCDR2 and a LCDR3 of a light chain variable region (VL) of SEQ ID NO: 37;
   b) the HCDR1, the HCDR2 and the HCDR3 of the VH of SEQ ID NO: 39 and the LCDR1, the LCDR2 and the LCDR3 of the VL of SEQ ID NO: 41;
   c) the HCDR1, the HCDR2 and the HCDR3 of the VH of SEQ ID NO: 43 and the LCDR1, the LCDR2 and the LCDR3 of the VL of SEQ ID NO: 41;
   d) the HCDR1, the HCDR2 and the HCDR3 of the VH of SEQ ID NO: 45 and the LCDR1, the LCDR2 and the LCDR3 of the VL of SEQ ID NO: 47;
   e) the HCDR1, the HCDR2 and the HCDR3 of the VH of SEQ ID NO: 49 and the LCDR1, the LCDR2 and the LCDR3 of the VL of SEQ ID NO: 51;
   f) the HCDR1, the HCDR2 and the HCDR3 of the VH of SEQ ID NO: 39 and the LCDR1, the LCDR2 and the LCDR3 of the VL of SEQ ID NO: 53;
   g) the HCDR1, the HCDR2 and the HCDR3 of the VH of SEQ ID NO: 55 and the LCDR1, the LCDR2 and the LCDR3 of the VL of SEQ ID NO: 57;
   h) the HCDR1, the HCDR2 and the HCDR3 of the VH of SEQ ID NO: 59 and the LCDR1, the LCDR2 and the LCDR3 of the VL of SEQ ID NO: 61;
   i) the HCDR1, the HCDR2 and the HCDR3 of the VH of SEQ ID NO: 63 and the LCDR1, the LCDR2 and the LCDR3 of the VL of SEQ ID NO: 65;
   j) the HCDR1, the HCDR2 and the HCDR3 of the VH of SEQ ID NO: 67 and the LCDR1, the LCDR2 and the LCDR3 of the VL of SEQ ID NO: 69; or
   k) the HCDR1, the HCDR2 and the HCDR3 of the VH of SEQ ID NO: 71 and the LCDR1, the LCDR2 and the LCDR3 of the VL of SEQ ID NO: 73.
149. The bispecific antibody or bispecific binding fragment of any one of clauses 135-148, wherein the first antigen-binding site that specifically binds CD79b comprises the HCDR1, the HCDR2, the HCDR3, the LCDR1, the LCDR2 and the LCDR3 of:
   a) SEQ ID NOs: 1, 2, 3, 4, 5 and 6, respectively;
   b) SEQ ID NOs: 13, 8, 9, 10, 11 and 12, respectively;
   c) SEQ ID NOs: 7, 8, 9, 10, 11 and 12, respectively;
   d) SEQ ID NOs: 14, 15, 16, 17, 5 and 6, respectively;
   e) SEQ ID NOs: 18, 8, 19, 20, 21 and 12, respectively;
   f) SEQ ID NOs: 22, 23, 24, 25, 5 and 6, respectively;
   g) SEQ ID NOs: 22, 26, 27, 28, 5 and 29, respectively; or
   h) SEQ ID NOs: 30, 31, 32, 33, 5 and 6, respectively.
150. The bispecific antibody or bispecific binding fragment of any one of clauses 135-149, wherein the first antigen-binding site that specifically binds CD79b comprises:
   a) the VH of SEQ ID NO: 35 and the VL of SEQ ID NO: 37;
   b) the VH of SEQ ID NO: 39 and the VL of SEQ ID NO: 41;
   c) the VH of SEQ ID NO: 43 and the VL of SEQ ID NO: 41;
   d) the VH of SEQ ID NO: 45 and the VL of SEQ ID NO: 47;
   e) the VH of SEQ ID NO: 49 and the VL of SEQ ID NO: 51;
   f) the VH of SEQ ID NO: 39 and the VL of SEQ ID NO: 53;
   g) the VH of SEQ ID NO: 55 and the VL of SEQ ID NO: 57;
   h) the VH of SEQ ID NO: 59 and the VL of SEQ ID NO: 61;
   i) the VH of SEQ ID NO: 63 and the VL of SEQ ID NO: 65;
   j) the VH of SEQ ID NO: 67 and the VL of SEQ ID NO: 69; or
   k) the VH of SEQ ID NO: 71 and the VL of SEQ ID NO: 73.
151. The bispecific antibody or bispecific binding fragment of any one of clauses 135-150, wherein the second antigen-binding site that specifically binds CD3 comprises:
   a) the HCDR1, the HCDR2 and the HCDR3 of the VH of SEQ ID NO: 97 and the LCDR1, the LCDR2 and the LCDR3 of the VL of SEQ ID NO: 99;
   b) the HCDR1, the HCDR2 and the HCDR3 of the VH of SEQ ID NO: 101 and the LCDR1, the LCDR2 and the LCDR3 of the VL of SEQ ID NO: 99;
   c) the HCDR1, the HCDR2 and the HCDR3 of the VH of SEQ ID NO: 103 and the LCDR1, the LCDR2 and the LCDR3 of the VL of SEQ ID NO: 99;
   d) the HCDR1, the HCDR2 and the HCDR3 of the VH of SEQ ID NO: 105 and the LCDR1, the LCDR2 and the LCDR3 of the VL of SEQ ID NO: 99; or
   e) the HCDR1, the HCDR2 and the HCDR3 of the VH of SEQ ID NO: 107 and the LCDR1, the LCDR2 and the LCDR3 of the VL of SEQ ID NO: 109.
152. The bispecific antibody or bispecific binding fragment of any one of clauses 135-151, wherein the second antigen-binding site that specifically binds CD3 comprises the HCDR1, the HCDR2, the HCDR3, the LCDR1, the LCDR2 and the LCDR3 of:
   a) SEQ ID NOs: 76, 77, 78, 79, 80 and 81, respectively;
   b) SEQ ID NOs: 76, 77, 75, 79, 80 and 81, respectively;
   c) SEQ ID NOs: 76, 77, 82, 79, 80 and 81, respectively; or
   d) SEQ ID NOs: 83, 84, 85, 86, 87 and 88, respectively.
153. The bispecific antibody or bispecific binding fragment of any one of clauses 135-152, wherein the second or third antigen-binding site that specifically binds CD3 comprises:
   a) the VH of SEQ ID NO: 97 and the VL of SEQ ID NO: 99;
   b) the VH of SEQ ID NO: 101 and the VL of SEQ ID NO: 99;
   c) the VH of SEQ ID NO: 103 and the VL of SEQ ID NO: 99;
   d) the VH of SEQ ID NO: 105 and the VL of SEQ ID NO: 99; or
   e) the VH of SEQ ID NO: 107 and the VL of SEQ ID NO: 109.
154. The bispecific antibody or bispecific binding fragment of any one of clauses 137-153, wherein the scFv comprises, from the N- to C-terminus, a VH, a linker (L) and a VL (VH-L-VL) or the VL, the linker (L) and the VH (VL-L-VH).
155. The bispecific antibody or bispecific binding fragment of any one of clauses 137-154, wherein the scFv comprises, from the N- to C-terminus, the VL, the linker (L) and the VH (VL-L-VH).
156. The bispecific antibody or bispecific binding fragment of clause 154 or 155, wherein the linker (L) comprises any one of amino acid sequence of SEQ ID NOs: 215-248.
157. The bispecific antibody or bispecific binding fragment of any one of clauses 154-156, wherein the linker (L) comprises an amino acid sequence of GGSEGKSSGSGSESKSTGGS (SEQ ID NO: 215).
158. The bispecific antibody or bispecific antigen-binding fragment of any one of clauses 154-157, wherein the first antigen-binding site specifically binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
159. The bispecific antibody or bispecific antigen-binding fragment of clause 158, wherein the first antigen-binding site specifically binds to CD79b with an affinity of about 1×10⁻¹¹-1×10⁻⁹ M.
160. The bispecific antibody or bispecific antigen-binding fragment of any one of clauses 154-159, wherein the antibody or antigen-binding fragment thereof are of IgG1, IgG2, IgG3, or IgG4 isotype.
161. The bispecific antibody or bispecific antigen-binding fragment of any of clauses 154-160 is an IgG1 isotype.
162. The bispecific antibody or bispecific binding fragment of clause 154, wherein the HC1 comprises the amino acid sequence of SEQ ID NO: 172, 176, or 180.
163. The bispecific antibody or bispecific binding fragment of clause 154 or 162, wherein the LC comprises the amino acid sequence of SEQ ID NO: 174, 178, or 182.
164. The bispecific antibody or bispecific binding fragment of any one of clauses 154, 162-163, wherein
   a) the HC1 comprises the amino acid sequence of SEQ ID NO: 172 and the LC comprises the amino acid sequence of SEQ ID NO: 174;
   b) the HC1 comprises the amino acid sequence of SEQ ID NO: 176 and the LC comprises the amino acid sequence of SEQ ID NO: 178;
   c) the HC1 comprises the amino acid sequence of SEQ ID NO: 180 and the LC comprises the amino acid sequence of SEQ ID NO: 182.
165. The bispecific antibody or bispecific binding fragment of any one of clauses 154, 162-164, wherein the HC2 comprises the amino acid sequence of SEQ ID NO: 164 or 189.
166. The bispecific antibody or bispecific binding fragment of any one of clauses 154, 162-165, wherein
   a) the HC1 comprises the amino acid sequence of SEQ ID NO: 172, the LC comprises the amino acid sequence of SEQ ID NO: 174, and the HC2 comprises the amino acid sequence of SEQ ID NO: 164;
   b) the HC1 comprises the amino acid sequence of SEQ ID NO: 176, the LC comprises the amino acid sequence of SEQ ID NO: 178, and the HC2 comprises the amino acid sequence of SEQ ID NO: 164;
   c) the HC1 comprises the amino acid sequence of SEQ ID NO: 180, the LC comprises the amino acid sequence of SEQ ID NO: 182, and the HC2 comprises the amino acid sequence of SEQ ID NO: 164;
   d) the HC1 comprises the amino acid sequence of SEQ ID NO: 172, the LC comprises the amino acid sequence of SEQ ID NO: 174, and the HC2 comprises the amino acid sequence of SEQ ID NO: 189;
   e) the HC1 comprises the amino acid sequence of SEQ ID NO: 176, the LC comprises the amino acid sequence of SEQ ID NO: 178, and the HC2 comprises the amino acid sequence of SEQ ID NO: 189; or
   f) the HC1 comprises the amino acid sequence of SEQ ID NO: 180, the LC comprises the amino acid sequence of SEQ ID NO: 182, and the HC2 comprises the amino acid sequence of SEQ ID NO: 189.
167. An bispecific antibody, or a bispecific binding fragment thereof, comprising a first antigen-binding site that specifically binds CD79b and a second antigen-binding site that specifically binds CD3.
168. The bispecific antibody or bispecific binding fragment of clause 167, wherein the first antigen-binding site that specifically binds CD79b comprises:
   a) the HCDR1, the HCDR2 and the HCDR3 of the VH of SEQ ID NO: 35 and the LCDR1, the LCDR2 and the LCDR3 of the VL of SEQ ID NO: 37;
   b) the HCDR1, the HCDR2 and the HCDR3 of the VH of SEQ ID NO: 39 and the LCDR1, the LCDR2 and the LCDR3 of the VL of SEQ ID NO: 41;
   c) the HCDR1, the HCDR2 and the HCDR3 of the VH of SEQ ID NO: 43 and the LCDR1, the LCDR2 and the LCDR3 of the VL of SEQ ID NO: 41;
   d) the HCDR1, the HCDR2 and the HCDR3 of the VH of SEQ ID NO: 45 and the LCDR1, the LCDR2 and the LCDR3 of the VL of SEQ ID NO: 47;
   e) the HCDR1, the HCDR2 and the HCDR3 of the VH of SEQ ID NO: 49 and the LCDR1, the LCDR2 and the LCDR3 of the VL of SEQ ID NO: 51;
   f) the HCDR1, the HCDR2 and the HCDR3 of the VH of SEQ ID NO: 39 and the LCDR1, the LCDR2 and the LCDR3 of the VL of SEQ ID NO: 53;
   g) the HCDR1, the HCDR2 and the HCDR3 of the VH of SEQ ID NO: 55 and the LCDR1, the LCDR2 and the LCDR3 of the VL of SEQ ID NO: 57;
   h) the HCDR1, the HCDR2 and the HCDR3 of the VH of SEQ ID NO: 59 and the LCDR1, the LCDR2 and the LCDR3 of the VL of SEQ ID NO: 61;
   i) the HCDR1, the HCDR2 and the HCDR3 of the VH of SEQ ID NO: 63 and the LCDR1, the LCDR2 and the LCDR3 of the VL of SEQ ID NO: 65;
   j) the HCDR1, the HCDR2 and the HCDR3 of the VH of SEQ ID NO: 67 and the LCDR1, the LCDR2 and the LCDR3 of the VL of SEQ ID NO: 69; or
   k) the HCDR1, the HCDR2 and the HCDR3 of the VH of SEQ ID NO: 71 and the LCDR1, the LCDR2 and the LCDR3 of the VL of SEQ ID NO: 73.
169. The bispecific antibody or bispecific binding fragment of clause 167 or 168, wherein the first antigen-binding site that specifically binds CD79b comprises the HCDR1, the HCDR2, the HCDR3, the LCDR1, the LCDR2 and the LCDR3 of:
   a) SEQ ID NOs: 1, 2, 3, 4, 5 and 6, respectively;
   b) SEQ ID NOs: 13, 8, 9, 10, 11 and 12, respectively;
   c) SEQ ID NOs: 7, 8, 9, 10, 11 and 12, respectively;
   d) SEQ ID NOs: 14, 15, 16, 17, 5 and 6, respectively;
   e) SEQ ID NOs: 18, 8, 19, 20, 21 and 12, respectively;
   f) SEQ ID NOs: 22, 23, 24, 25, 5 and 6, respectively;
   g) SEQ ID NOs: 22, 26, 27, 28, 5 and 29, respectively; or
   h) SEQ ID NOs: 30, 31, 32, 33, 5 and 6, respectively.
170. The bispecific antibody or bispecific binding fragment of any one of clauses 167-169, wherein the first antigen-binding site that specifically binds CD79b comprises:
   a) the VH of SEQ ID NO: 35 and the VL of SEQ ID NO: 37;
   b) the VH of SEQ ID NO: 39 and the VL of SEQ ID NO: 41;
   c) the VH of SEQ ID NO: 43 and the VL of SEQ ID NO: 41;
   d) the VH of SEQ ID NO: 45 and the VL of SEQ ID NO: 47;
   e) the VH of SEQ ID NO: 49 and the VL of SEQ ID NO: 51;
   f) the VH of SEQ ID NO: 39 and the VL of SEQ ID NO: 53;
   g) the VH of SEQ ID NO: 55 and the VL of SEQ ID NO: 57;
   h) the VH of SEQ ID NO: 59 and the VL of SEQ ID NO: 61;
   i) the VH of SEQ ID NO: 63 and the VL of SEQ ID NO: 65;
   j) the VH of SEQ ID NO: 67 and the VL of SEQ ID NO: 69; or
   k) the VH of SEQ ID NO: 71 and the VL of SEQ ID NO: 73.
171. The bispecific antibody or bispecific binding fragment of any one of clauses 167-170, wherein the second antigen-binding site that specifically binds CD3 comprises:
   a) the HCDR1, the HCDR2 and the HCDR3 of the VH of SEQ ID NO: 97 and the LCDR1, the LCDR2 and the LCDR3 of the VL of SEQ ID NO: 99;
   b) the HCDR1, the HCDR2 and the HCDR3 of the VH of SEQ ID NO: 101 and the LCDR1, the LCDR2 and the LCDR3 of the VL of SEQ ID NO: 99;
   c) the HCDR1, the HCDR2 and the HCDR3 of the VH of SEQ ID NO: 103 and the LCDR1, the LCDR2 and the LCDR3 of the VL of SEQ ID NO: 99;
   d) the HCDR1, the HCDR2 and the HCDR3 of the VH of SEQ ID NO: 105 and the LCDR1, the LCDR2 and the LCDR3 of the VL of SEQ ID NO: 99; or
   e) the HCDR1, the HCDR2 and the HCDR3 of the VH of SEQ ID NO: 107 and the LCDR1, the LCDR2 and the LCDR3 of the VL of SEQ ID NO: 109.
172. The bispecific antibody or bispecific binding fragment of any one of clauses 167-171, wherein the second antigen-binding site that specifically binds CD3 comprises:
   a) SEQ ID NOs: 76, 77, 78, 79, 80 and 81, respectively;
   b) SEQ ID NOs: 76, 77, 75, 79, 80 and 81, respectively;
   c) SEQ ID NOs: 76, 77, 82, 79, 80 and 81, respectively; or
   d) SEQ ID NOs: 83, 84, 85, 86, 87 and 88, respectively.
173. The bispecific antibody or bispecific binding fragment of any one of clauses 167-172, wherein the second antigen-binding site that specifically binds CD3 comprises
   a) the VH of SEQ ID NO: 97 and the VL of SEQ ID NO: 99;
   b) the VH of SEQ ID NO: 101 and the VL of SEQ ID NO: 99;
   c) the VH of SEQ ID NO: 103 and the VL of SEQ ID NO: 99;
   d) the VH of SEQ ID NO: 105 and the VL of SEQ ID NO: 99; or
   e) the VH of SEQ ID NO: 107 and the VL of SEQ ID NO: 109.
174. The bispecific antibody or bispecific antigen-binding fragment of any one of clauses 167-173, wherein the first antigen-binding site specifically binds to residues 30-42 (SEDRYRNPKGSAC; SEQ ID NO: 253), residues 50-52 (PRF), residues 81-86 (EMENP; SEQ ID NO: 254), and/or residues 144-148 (GFSTL; SEQ ID NO: 255) of human CD79b.
175. The bispecific antibody or bispecific antigen-binding fragment of clause 174, wherein the first antigen-binding site specifically binds to CD79b with an affinity of about 1×10⁻¹¹-1×10⁻⁹ M.
176. The bispecific antibody or bispecific antigen-binding fragment of any one of clauses 167 to 175, wherein the antibody or antigen-binding fragment thereof are of IgG1, IgG2, IgG3, or IgG4 isotype.
177. The bispecific antibody or bispecific antigen-binding fragment of any of clauses 167 to 176 is an IgG1 isotype.
178. An synthetic polynucleotide encoding the bispecific antibody or bispecific binding fragment of any one of clauses 167 to 177.
179. The synthetic polynucleotide of clause 178, wherein the polynucleotide comprises a sequence encoding an antigen-binding site that specifically binds CD79b, said sequence comprising
   a) a VH-encoding sequence of SEQ ID NO: 36 and a VL-encoding sequence of SEQ ID NO: 38 or 213;
   b) a VH-encoding sequence of SEQ ID NO: 40 and a VL-encoding sequence of SEQ ID NO: 42;
   c) a VH-encoding sequence of SEQ ID NO: 44 and a VL-encoding sequence of SEQ ID NO: 34;
   d) a VH-encoding sequence of SEQ ID NO: 46 and a VL-encoding sequence of SEQ ID NO: 48 or 214;
   e) a VH-encoding sequence of SEQ ID NO: 50 and a VL-encoding sequence of SEQ ID NO: 52;
   f) a VH-encoding sequence of SEQ ID NO: 40 and a VL-encoding sequence of SEQ ID NO: 54;
   g) a VH-encoding sequence of SEQ ID NO: 56 and a VL-encoding sequence of SEQ ID NO: 58;
   h) a VH-encoding sequence of SEQ ID NO: 60 and a VL-encoding sequence of SEQ ID NO: 62;
   i) a VH-encoding sequence of SEQ ID NO: 64 and a VL-encoding sequence of SEQ ID NO: 66;
   j) a VH-encoding sequence of SEQ ID NO: 68 and a VL-encoding sequence of SEQ ID NO: 70; or
   k) a VH-encoding sequence of SEQ ID NO: 72 and a VL-encoding sequence of SEQ ID NO: 74.
180. The synthetic polynucleotide of clause 178 or 179, wherein the polynucleotide comprises a sequence encoding an antigen-binding site that specifically binds CD3, said sequence comprising
   a) a VH-encoding sequence of SEQ ID NO: 98 and a VL-encoding sequence of SEQ ID NO: 100;
   b) a VH-encoding sequence of SEQ ID NO: 102 and a VL-encoding sequence of SEQ ID NO: 100;
   c) a VH-encoding sequence of SEQ ID NO: 104 and a VL-encoding sequence of SEQ ID NO: 100;
   d) a VH-encoding sequence of SEQ ID NO: 106 and a VL-encoding sequence of SEQ ID NO: 100; or
   e) a VH-encoding sequence of SEQ ID NO: 108 and a VL-encoding sequence of SEQ ID NO: 110.
181. The synthetic polynucleotide of any one of clauses 178-180, wherein the polynucleotide comprises a sequence encoding an HC1 comprising the nucleotide sequence of SEQ ID NO: 173, 177, or 181.
182. The synthetic polynucleotide of any one of clauses 178-181, wherein the polynucleotide comprises a sequence encoding a LC comprising the nucleotide sequence of SEQ ID NO: 175, 179, or 183.
183. The synthetic polynucleotide of any one of clauses 178-182, comprising
   a) an HC1-encoding sequence of SEQ ID NO: 173 and a LC-encoding sequence of SEQ ID NO: 175;
   b) an HC1-encoding sequence of SEQ ID NO: 177 and a LC-encoding sequence of SEQ ID NO: 179; or
   c) an HC1-encoding sequence of SEQ ID NO: 181 and a LC-encoding sequence of SEQ ID NO: 183.
184. The synthetic polynucleotide of any one of clauses 178-183, wherein the polynucleotide comprises a sequence encoding an HC2 comprising the nucleotide sequence of SEQ ID NO: 165 or 190.
185. The synthetic polynucleotide of any one of clauses 178-184, comprising
   a) an HC1-encoding sequence of SEQ ID NO: 173 and a LC-encoding sequence of SEQ ID NO: 175 and an HC2-encoding sequence of SEQ ID NO: 165;
   b) an HC1-encoding sequence of SEQ ID NO: 177 and a LC-encoding sequence of SEQ ID NO: 179 and an HC2-encoding sequence of SEQ ID NO: 165;
   c) an HC1-encoding sequence of SEQ ID NO: 181 and a LC-encoding sequence of SEQ ID NO: 183 and an HC2-encoding sequence of SEQ ID NO: 165;
   d) an HC1-encoding sequence of SEQ ID NO: 173 and a LC-encoding sequence of SEQ ID NO: 175 and an HC2-encoding sequence of SEQ ID NO: 190;
   e) an HC1-encoding sequence of SEQ ID NO: 177 and a LC-encoding sequence of SEQ ID NO: 179 and an HC2-encoding sequence of SEQ ID NO: 190; or
   f) an HC1-encoding sequence of SEQ ID NO: 181 and a LC-encoding sequence of SEQ ID NO: 183 and an HC2-encoding sequence of SEQ ID NO: 190.
186. A pharmaceutical composition comprising the bispecific antibody or bispecific binding fragment of any one of clauses 154 to 177, or the polynucleotide of any one of clauses 178-185, and a pharmaceutically acceptable carrier.
187. An isolated cell expressing the bispecific antibody or bispecific binding fragment of any one of clauses 154 to 177.
188. The cell of clause 187 wherein the cell is a hybridoma.
189. The cell of clause 187 wherein the antibody is recombinantly produced.
190. A method for treating cancer in a subject in need thereof, said method comprising administering to the subject a therapeutically effective amount of the bispecific antibody or bispecific binding fragment of any one of clauses 154 to 177, the polynucleotide of any one of clauses 178-185, or the pharmaceutical composition of clause 186.
191. The method of clause 190, wherein the bispecific antibody or bispecific binding fragment or the pharmaceutical composition is administered for a time sufficient to treat the cancer.
192. A method for inhibiting growth or proliferation of a cancer cell, said method comprising administering to said cell an effective amount of the bispecific antibody or bispecific binding fragment of any one of clauses 154 to 177, the polynucleotide of any one of clauses 178-185, or the pharmaceutical composition of clause 186, wherein said effective amount is sufficient to inhibit the growth or proliferation of said cancer cell.
193. The method of clause 192, wherein said cancer cell is in a subject and the bispecific antibody or bispecific binding fragment or the pharmaceutical composition is administered to the subject.
194. The method of clause 193, wherein said administration is conducted ex vivo.
195. A method of redirecting a T cell to CD79b-expressing cancer cells in a subject in need thereof, said method comprising administering to said subject a therapeutically effective amount of the bispecific antibody or bispecific binding fragment of any one of clauses 154 to 177, the polynucleotide of any one of clauses 178-185, or the pharmaceutical composition of clause 186.
196. The method of clause 195, wherein the said therapeutically effective amount is sufficient to direct said T cell response to said cancer cells.
197. The method of any one of clauses 190-196, wherein the cancer is a hematological cancer.
198. The method of clause 197 wherein the hematological cancer is a CD79b-expressing B cell cancer.
199. The method of clause 198 wherein the CD79b-expressing B cell cancer is a B-cell lymphoma or a non-Hodgkin lymphoma.
200. The method of clause 199 wherein the CD79b-expressing B cell cancer is a diffuse large B-cell lymphoma (DLBCL), mantle cell lymphoma (MCL), marginal zone lymphoma (MZL), or follicular lymphoma (FL).
201. The method of any one of clauses 190-200, wherein the cancer is relapsed, refractory, or malignant cancer, or any combination thereof.
202. The method of any one of clauses 190-201 further comprising administering a second therapeutic agent.
203. The method of clause 202 wherein the second therapeutic agent is a surgery, chemotherapy, androgen deprivation therapy or radiation, or any combination thereof.
204. A method for generating the bispecific antibody or bispecific binding fragment of any one of clauses 154 to 177, wherein said method comprises culturing the cell of any one of clauses 187-189 and isolating said bispecific antibody or bispecific binding fragment.
205. A kit comprising (i) the bispecific antibody or bispecific binding fragment of any one of clauses 154 to 177 and/or a polynucleotide of any one of clauses 178-185, and (ii) packaging for the same.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### Example 1: Target Validation

B-cell non-Hodgkin lymphoma (B-NHL) tumor cells express B-cell-related antigens (i.e., CD20, CD22, CD79b, and CD19) on their cell surfaces at heterogeneous levels. As these antigens are expressed exclusively in the B-cell lineage, and not in other cell lineages or tissues, antibody therapies targeting these antigens have been actively developed in patients with B-NHL.

CD79b is expressed on the surface of B cells from pre-B through memory B-cell stage and, in cancer, is highly expressed in mantle cell lymphoma (MCL), diffuse large B-cell lymphoma (DLBCL), follicular lymphoma (FL), and marginal zone lymphoma (MZL). While CD79b expression is low in chronic lymphocytic leukemia (CLL), increased CD79b surface expression correlates with acalabrutinib resistance in patients with CLL (13).

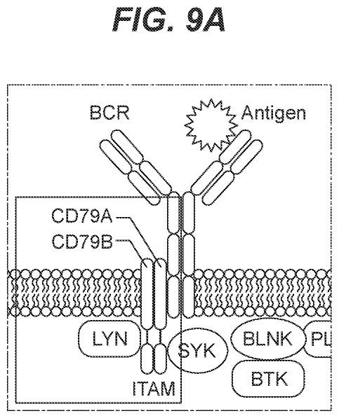

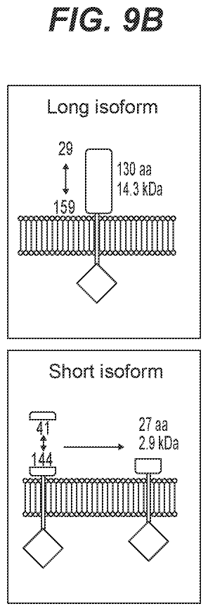

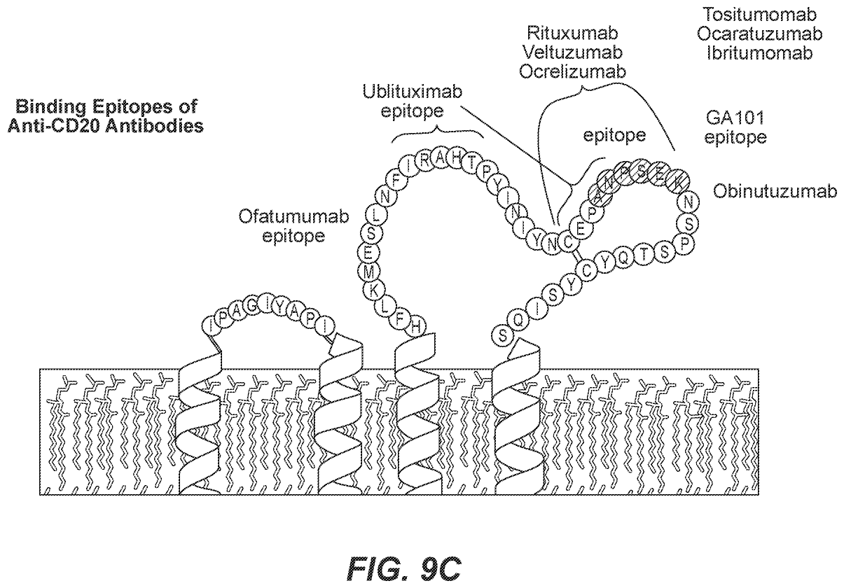

CD79b (also known as immunoglobulin [Ig]-β or B29) forms a disulfide-linked heterodimer with CD79a (also known as Ig-α or MB-1), and in association with membrane-bound immunoglobulins (mIgs), forms the B-cell receptor (BCR). The CD79 heterodimer constitutes the signaling component of the BCR (FIG. 9A). Upon antigen recognition, specific tyrosine residues within the cytoplasmic immunoreceptor tyrosine-based activation motif (ITAM) domain of CD79a/b are phosphorylated by Src-family tyrosine kinases (e.g., LYN, FYN, BLK), which leads to the recruitment and activation of SH2-containing tyrosine kinases (e.g., Syk) and initiation of the signaling cascades that control B-cell immune responses. BCR signaling regulates a variety of functions in mature B cells, such as signal transduction in response to antigen stimulation, processing, and presentation. CD79b mutations, identified in approximately 30% of activated B cell (ABC) DLBCLs and 3% of germinal center B cell (GCB) DLBCL, while CD79a mutations are less common, ranging from 2.9% to 4% of cases ABC DLBCL (14). CD79b mutations have been described as oncogenic drivers in DLBCL by leading to constitutive activation of the nuclear factor kappa-light-chain-enhancer of activated B cells (NF-κB) pathway. In addition, a large proportion of DLBCL tumors are dependent on CD79b expression for survival, independent of its mutational status (15-17). Therefore, CD79b is an attractive target for a T-cell redirection approach, as the development of resistance to CD79b-targeted agents through antigen loss may be less likely.

The CD79b protein consists of 229 amino acids characterized by an extracellular domain (ECD) containing a signal peptide, a V-type Ig-like domain, a transmembrane (TM) domain, and an ITAM-containing cytoplasmic domain (FIG. 9A). Two splice isoforms of CD79b exist (FIG. 9B), with the long CD79b isoform being the prominent form in DLBCL. The truncated CD79b isoform lacks a portion of the ECD that contains the cysteine residues involved in the interaction with Igs to stabilize its TM portion and promote BCR surface expression. It is detected at very low expression in DLBCL tumors, and when present in Burkitt lymphomas it is not efficiently transported to the plasma membrane (18). CD79b shares very little homology with CD79a as shown by FASTA analysis (i.e., 29% identity). Moreover, sequence alignment of CD79b orthologues revealed that while the TM and the intracellular domain of CD79b are highly conserved among various species, the sequence homology of the ECD is significantly lower (Table 46).

CD20 is a transmembrane protein of the membrane-spanning 4-domain family A (MS4A) protein family involved in B-cell activation and differentiation. CD20 is expressed on all mature B-cells and most B-NHL tumors (21). The CD20 protein consists of 4 hydrophobic TM domains, 1 intracellular domain, and 2 ECDs (large and small loops) with both N- and C-termini residing within the cytosol (FIG. 9C) (22). While 3 different transcripts of CD20 have been identified, they are all translated into identical full-length CD20 protein. Moreover, other alternative transcripts have been identified in malignant B cells alongside CD20 full-length protein, but these truncated forms represent only a very minor fraction of total CD20 protein (<5%; 23). It has been proposed that they might impair binding of anti-CD20 monoclonal antibodies (mAbs) in cellular models (23,24). Epstein-Barr virus (EBV) transformation might modify the CD20 splicing profile and contribute to generation of CD20 variant transcripts (23). Three different phosphorylation patterns of CD20 have been identified, and CD20 phosphorylation was reported to be higher in proliferating malignant than in resting B cells (25). CD20 is organized in the plasma membrane as multimeric molecular complexes with other cell-surface and cytoplasmic proteins involved in BCR-activated calcium entry and contributing to signal transduction and B-cell proliferation (26).

The CD79b and CD20 proteins are specific to the B-cell lineage and their pattern of expression is closely similar to that of some of the most common B-cell antigens, starting from early committed B-cell progenitors (early pre-B stage) until mature B-cell stage. Both proteins are undetectable in terminally differentiated plasma cells.

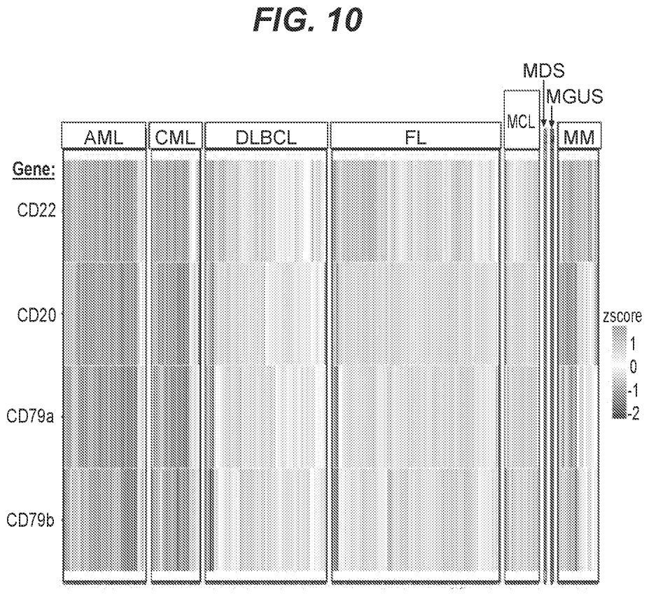

In cancer, CD79b and CD20 were found to be highly expressed in several B-NHL such as DLBCL, FL, and MCL (FIG. 10).

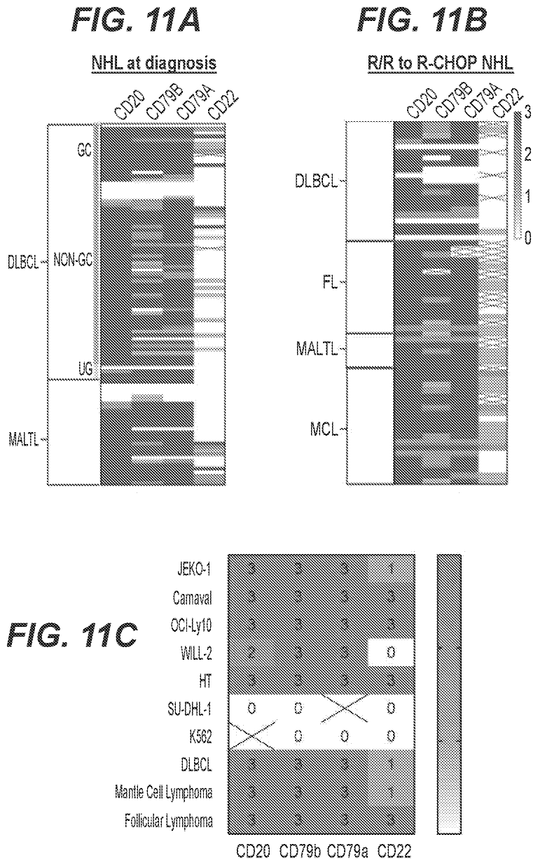

To confirm the expression of the targets of interest at the protein level, CD20, CD79b, CD79a, and CD22 immunohistochemistry (IHC) staining of formalin-fixed, paraffin-embedded (FFPE) tumor sections derived from B-NHL patients collected at diagnosis (FIG. 11A) or patients R/R to rituximab-cyclophosphamide-hydroxydaunorubicin-oncovin-prednisone/prednisolone regimen (R-CHOP; FIG. 11B) was performed. Expression of both CD20 and CD79b was confirmed across samples. Intensity of CD79b expression was more variable as compared to CD20. However, underlying IHC detection limitations of the anti-CD79b antibody should be taken into consideration interpreting these data, especially in cases when CD79a is expressed, which requires CD79b for efficient cell surface expression.

While the IHC results show high CD20 expression across B-NHL, recent reports showed that several CD20 gene mutations resulting in loss of CD20 expression were detected in patient samples at clinical progression on CD20 x CD3 bispecifics, suggesting potential target-antigen-dependent disease escape (27). These data overall support the hypothesis that dual targeting should provide a therapeutic benefit for a broad patient population. Given the expression profile of targeted tumor antigens and reported tumor dependency on CD79b expression, combined targeting of CD79b with CD20 might provide an inflection point in the management of B-cell malignancies.

### Example 2: Immunization Protocol and V Gene Recovery of CD79b Binders

The following protocol was used to prepare CD79b monoclonal antibodies (mAbs).

### AB239

A human immunoglobulin transgenic mouse strain (Ablexis®; AlivaMab, LLC.) was used to develop human CD79b monoclonal antibodies. The Ablexis® mice contained a chimeric human/mouse IgH locus (comprising of 32 human V alleles, 27 human D alleles and 6 human J alleles in natural configuration linked to the mouse C_{H} locus) together with fully human IgL locus (comprising of 18 Vκ alleles and 5 Jκ alleles and/or 29 V_{λ} alleles and 7 J_{λ} alleles linked to appropriate mouse Cλ or Cκ). Accordingly, the mice contained an inactivated endogenous Ig locus, and in response to immunization, the introduced human heavy and light chain transgenes undergo class switching and somatic mutation to generate high affinity human IgG monoclonal antibodies. The preparation and use of Ablexis®, and the genomic modifications carried by such mice, is described in U.S. Patent Pub. No. 2013/0167256.

When immunized with recombinant human CD79b (rhCD79b), this transgenic mouse produced human IgG antibodies specific to human CD79b.

For HYB650, the immunization strategy in Ablexis® kappa mice included repetitive immunizations multiple sites+intraperitoneal (RIMMS+IP) injections of rhCD79b (R&D Cas 9687-CD Lot: TLS021805A) in CL413 (InvivoGen cat #vac-c1413-5) (days 42, 49, and 56) or Sigma (Sigma, Catalog S6322) (days 72, 79, 86, and 114). On day 129, after sufficient titers were reached, mice were given a final boost of rhCD79b (R&D Cas 9687-CD Lot: TLS021805A)+CL413 (InvivoGen cat #vac-c1413-5)+CD40 (R&D cat #MAB440; lot: AHY181704A) 7 days prior to sorting. Spleens and mandibular, accessory mandibular, superficial parotid, proper axillary, accessory axillary, subiliac, sciatic, popliteal, gastric, pancreaticodoudenal, jejunal, and medial iliac lymph nodes were harvested and antigen-positive B cells were isolated by Fluorescence-activated cell sorting (FACS). Ten 384-well plates of sorted B cell supernatants were screened by cell-based MSD to identify mAbs with specific binding to human CD79b expressing SU-DHL-10 cells (CD79a/b expressing primary cell lines, AG000002270). Positive clones were sequenced, cloned and expressed in small scale.

### AB241

A human immunoglobulin transgenic mouse strain (Ablexis®; AlivaMab, LLC.) was used to develop human CD79b monoclonal antibodies. The Ablexis® mice contained a chimeric human/mouse IgH locus (comprising of 32 human V alleles, 27 human D alleles and 6 human J alleles in natural configuration linked to the mouse C_{H} locus) together with fully human IgL locus (comprising of 18 Vκ alleles and 5 Jκ alleles and/or 29 V_{λ} alleles and 7 J_{λ} alleles linked to appropriate mouse Cλ or Cκ). Accordingly, the mice contained an inactivated endogenous Ig locus, and in response to immunization, the introduced human heavy and light chain transgenes undergo class switching and somatic mutation to generate high affinity human IgG monoclonal antibodies. The preparation and use of Ablexis®, and the genomic modifications carried by such mice, is described in U.S. Patent Pub. No. 2013/0167256.

When immunized with recombinant human CD79b (rhCD79b), this transgenic mouse produced human IgG antibodies specific to human CD79b.

For HYB649, the immunization strategy in Ablexis® kappa mice consisted of RIMMS+IP injections rhCD79b (R&D Cas 9687-CD Lot: TLS021805A) in sigma adjuvant (Sigma, Catalog S6322) (days 0, 8, 13, and 20). On day 31, after sufficient titers were reached, mice were given a final boost of rhCD79b (R&D Cas 9687-CD Lot: TLS021805A)+anti-msCD40 (R&D cat #MAB440; lot: AHY181704A) 4 days prior to fusion. Spleens and mandibular, accessory mandibular, superficial parotid, proper axillary, accessory axillary, subiliac, sciatic, popliteal, gastric, pancreaticodoudenal, jejunal, and medial iliac lymph nodes were harvested and used to generate hybridomas. Sixty plates of hybridoma supernatants were screened by cell-based MSD to identify mAbs which exhibited binding to rhCD79b. After further confirmatory screenings, hybridoma supernatants from both screens that exhibited binding specific to human CD79b expressing SU-DHL-4 & SU-DHL-10 cells (CD79a/b expressing primary cell lines, AG000002269 & AG000002270, respectively) were sequenced, cloned and expressed in small scale.

### V Region Cloning

B-cells were lysed in RealTime Ready Cell Lysis Buffer (Roche) and the B cell lysate was used directly for cDNA synthesis using the Smarter cDNA synthesis kit (Clontech, Mount View, CA). To facilitate cDNA synthesis, oligodT was used to prime reverse transcription of all messenger RNAs followed by “5′ capping” with a Smarter IIA oligonucleotide. Subsequent amplification of the VH and VL fragments was performed using a 2-step PCR amplification using 5′ primers targeting the Smarter IIA cap and 3′ primers targeting consensus regions in CH1. Briefly, each 50 μl PCR reaction consists of 10 μM of forward and reverse primer mixes, 25 μl of PrimeStar Max DNA polymerase premix (Clontech), 2 μl of unpurified cDNA, and 21 μl of double-distilled H₂O. The cycling program starts at 94° C. for 3 min, followed by 35 cycles (94° C. for 10 Sec, 55° C. for 1 min, 68° C. for 1 min), and ends at 72° C. for 7 min. The second round PCR was performed with VL and VH 2nd round primers containing 15 bp complementary extensions that “overlap” respective regions in their respective Lonza mother vector (VH and VL). Second round PCR was performed with the following program: 94° C. for 3 min; 35 cycles (94° C. for 10 Sec, 65° C. for 1 min, 72° C. for 1 min), and ends at 72° C. for 7 min. In-Fusion® HD Cloning Kit (Clonetech, U.S.A.) was used for directional cloning of VL gene into Lonza huIgK or Lambda vector and VH gene into Lonza huIgG1 vector. To facilitate In-Fusion® HD Cloning, PCR products were treated with Cloning Enhancer before In-Fusion HD Cloning. Cloning and transformation were performed according to manufacturer's protocol (Clonetech, U.S.A.). Mini-prep DNAs were subjected to Sanger sequencing to confirm that complete V-gene fragments were obtained. The DNA plasmid DNA or glycerol stocks were sequenced at GENEWIZ using pre-designed primers to obtain v-region sequences. The resulting .abi files of V-region sequences were collected and analyzed by a Sanger V-region sequence analysis program. All V genes were cloned into the Lonza-based expression vector carrying the appropriate constant region of the desired human antibody isotype IgG1 AAS. A total of 147 antibodies were successfully cloned and proceeded for further characterization from Ablexis Mice.

### Example 3: Expression and Purification of Bispecific CD79bxCD3 and Trispecific CD79bxCD20xCD3 Antibodies

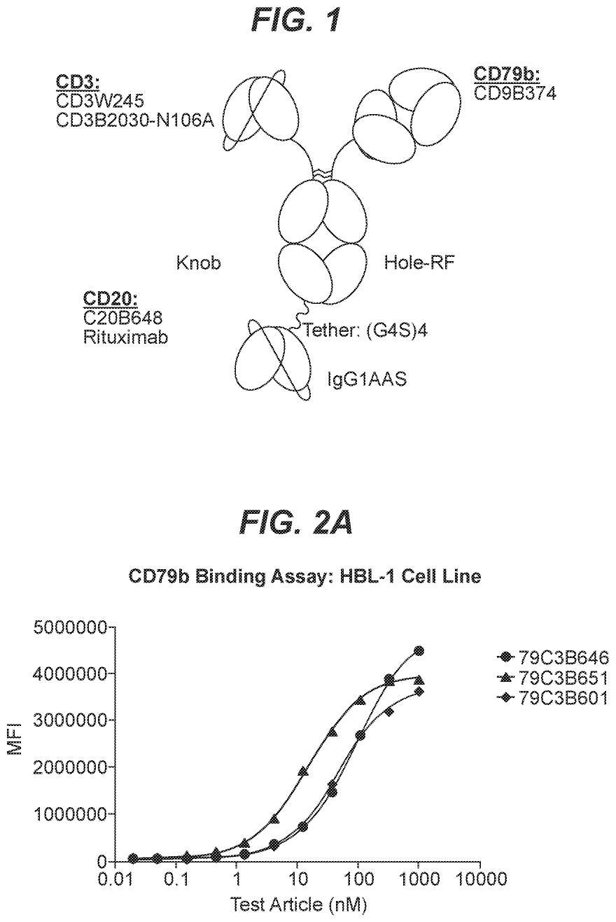

The CD79bxCD3 bispecific antibody (bsAb) is an immunoglobulin (Ig) G1 bispecific antibody that can bind simultaneously or individually to the cluster of differentiation (CD) 3 receptor complex on T lymphocytes and to CD79b on B lymphocytes. The CD79bxCD20xCD3 trispecific antibody is an immunoglobulin (Ig) G1 trispecific antibody that can bind simultaneously or individually to the CD3 receptor complex on T lymphocytes, and to the CD20 receptor complex on B lymphocytes and to the CD79b receptor complex on B lymphocytes. The antibody has mutations which reduce Fc binding to a Fcγ receptor and heterodimerization has been enhanced using the knobs-in-holes platform mutations. The trispecific antibody was developed to evaluate the therapeutic potential of dual targeting CD20 and CD79b for T cell redirection. An illustration of an exemplary CD79bxCD20xCD3 antibody is depicted in FIG. 1.

The antibodies were expressed in ExpiCHO-S™ cells (ThermoFisher Scientific; Waltham, MA, Cat #A29127) by transient transfection with purified plasmid DNA following the manufacturer's recommendations. Briefly, ExpiCHO-S™ cells were maintained in suspension in ExpiCHO™ expression medium (ThermoFisher Scientific, Cat #A29100) in an orbital shaking incubator set at 37° C., 8% CO₂ and 125 RPM. The cells were passaged and diluted prior to transfection to 6.0×10⁶ cells per ml, maintaining cell viability at 99.0% or better. Transient transfections were done using the ExpiFectamine™ CHO transfection kit (ThermoFisher Scientific, Cat #A29131). For each ml of diluted cells to be transfected, 0.5 microgram of bispecific encoding DNA (HC1:HC2:LC=1:2:2) and 0.5 microgram of pAdVAntage DNA (Promega, Cat #E1711) was used and diluted into OptiPRO™ SFM complexation medium. ExpiFectamine™ CHO reagent was used at a 1:4 ratio (v/v, DNA:reagent) and diluted into OptiPRO™. The diluted DNA and transfection reagent were combined for one minute, allowing DNA/lipid complex formation, and then added to the cells. After overnight incubation, ExpiCHO™ feed and ExpiFectamine™ CHO enhancers were added to the cells as per the manufacturer's Standard protocol. Cells were incubated with orbital shaking (125 rpm) at 37° C. for seven days prior to harvesting the culture broth. The culture supernatant from the transiently transfected ExpiCHO-S™ cells was clarified by centrifugation (30 min, 3000 ref) followed by filtration (0.2 μm PES membrane, Corning; Corning, NY).

The filtered cell culture supernatant was loaded onto a pre-equilibrated (1×DPBS, pH 7.2) MabSelect Sure Protein A column (GE Healthcare) using an AKTAXpress chromatography system. After loading, the column was washed with 10 column volumes of 1×DPBS, pH7.2. The protein was eluted with 10 column volumes of 0.1 M sodium (Na)-Acetate, pH 3.5. Protein fractions were neutralized immediately by the addition of 2.5 M Tris HC1, pH 7.2 to 20% (v/v) of the elution fraction volume. Peak fractions were pooled and loaded onto a CH1 column (Thermofisher). After loading, the column was washed with 10 column volumes of 1×DPBS, pH7.2. The protein was eluted with 10 column volumes of 0.1 M sodium (Na)-Acetate, pH 3.5. Protein fractions were partially neutralized by the addition of 2.5 M Tris HC1, pH 7.2 to 15% (v/v) of the final volume. The high molecular weight species were removed by preparative size exclusion chromatography (SEC) using Superdex 200 (GE Healthcare). Post sample injection, the column was developed with 1×DPBS and the major peak fractions were pooled, dialyzed into 10 mM Histidine, pH6.5 and filtered (0.2 m).

The concentration of purified protein was determined by absorbance at 280 nm on a Dropsense spectrophotometer. The quality of the purified protein was assessed by cSDS and analytical size exclusion HPLC (Agilent HPLC system). The endotoxin level was measured using a turbidometric LAL assay (Pyrotell®-T, Associates of Cape Cod; Falmouth, MA).

### Example 4: Bispecific and Trispecific Antibodies Binding Characterization

### Binding of Bispecific CD79xCD3 Antibodies on CD79⁺ Target Cells

The binding affinity of the CD79b binding arm of the CD79x CD3 bispecific molecules were assessed using cell lines that were validated by flow cytometry to have different endogenous expression levels of CD79b on the cell surface, shown in Table 7.

TABLE 7
CD79b Antigen Density of B Lymphoma Cell Lines

| | | CD79b Antigen Density |
|---|---|---|
| Cell Line | Cell Type | (Antigen Number/cell) |
| HBL-1 | Diffuse large B-cell lymphoma line | 429,649 |
| OCI-LY-10 | Diffuse large B-cell lymphoma line | 38,885 |
| CARNAVAL | Diffuse large B-cell lymphoma line | 98,176 |
| WILL2 | Diffuse large B-cell lymphoma line | 3,824 |

Diffuse large B-cell lymphoma cell lines were incubated for 1 hour with CD79bxCD3 test molecules 79C3B646, 79C3B651, and 79C3B601 (1 uM starting concentration at 1:3 serial dilutions) at 37° C. All cells were washed with BD stain buffer (BD Biosciences; Cat #554657), centrifuged at 1200 RPM for 3 mins, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:200 dilution along with Aqua Fixable Live/Dead stain (Invitrogen; Cat #L34957) at a 1:400 dilution. All cells were washed with BD stain buffer (BD Biosciences; Cat #554657), centrifuged at 1200 RPM for 3 minutes, with supernatant discarded. Cells were analyzed using Intellicyt (Sartorius) flow cytometer and mean fluorescent intensity (MFI) was generated using Forcyt software (Sartorius). MFI was graphed and EC50 values generated using GraphPad PRISM v.8. Dose response curves were generated by transforming the x axis values using the formula x=lox. Data were then graphed using non-linear regression curve fit analysis “log(agonist) vs. response-variable slope (four parameter)”.

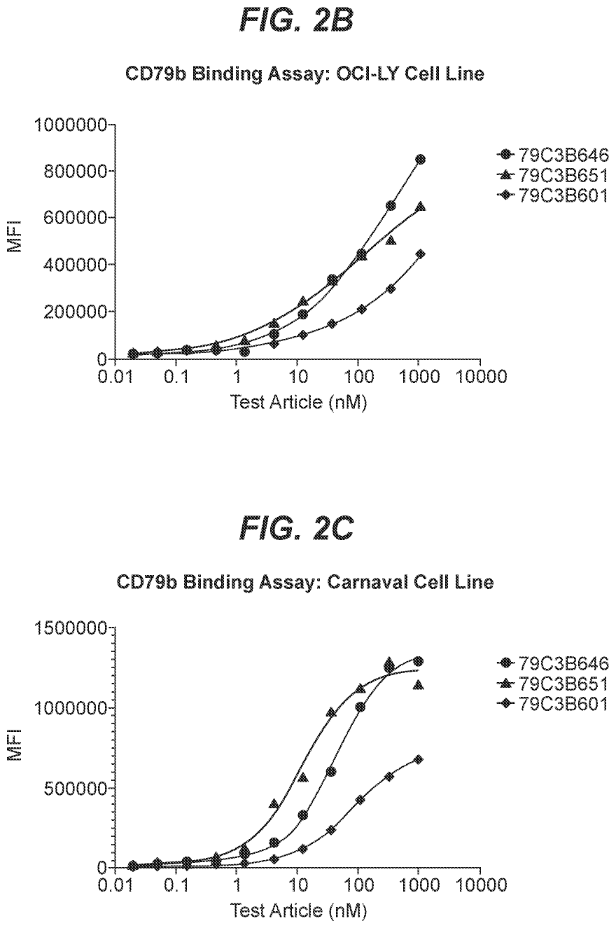

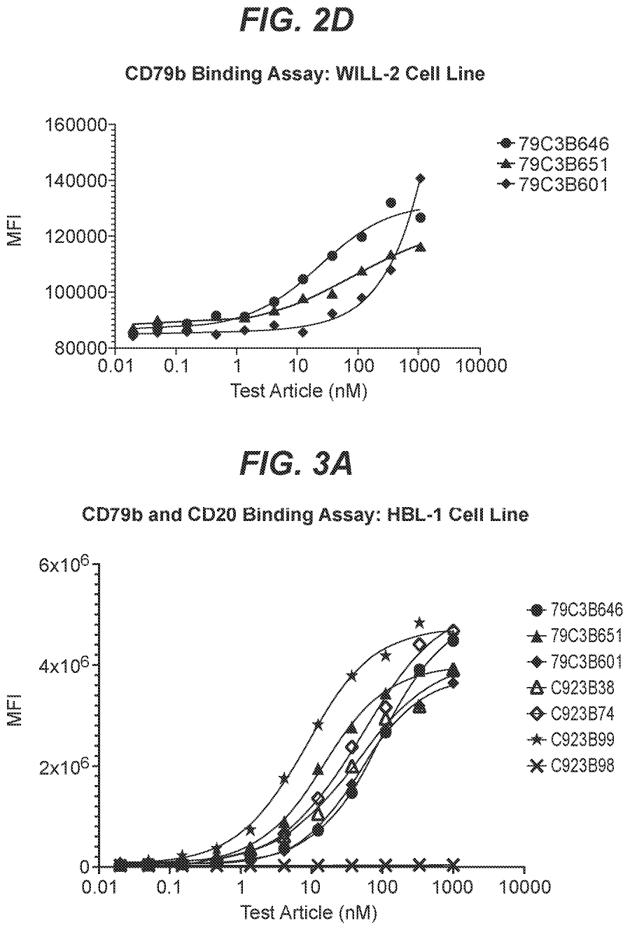

All CD79bxCD3 molecules showed good binding on cell lines expressing endogenous CD79b on the cell surface, with the CD79b binding arm of construct 79C3B651 showing the highest binding affinity across all tested cell lines, shown in FIGS. 2A-2D and Table 8.

TABLE 8
CD79bxCD3 Bispecifics Cell Binding EC50 Values

| | HBL-1 EC50 | OCI-LY10 EC50 | Carnaval | WILL-2 |
|---|---|---|---|---|
| | (nM) | (nM) | EC50 (nM) | EC50 (nM) |
| 79C3B646 | 97 | undetermined | 44 | undetermined |
| 79C3B651 | 15 | undetermined | 12 | undetermined |
| 79C3B601 | 48 | undetermined | 89 | undetermined |

Note:
For OCI-Ly10 and WILL-2 cell lines, antibody binding did not reach the plateau and therefore EC50 value could not be determined. On Table 8, the EC50 column is listed it as “undetermined” for these cell lines.

Binding of Trispecific CD79xCD20xCD3 Antibodies on CD79b⁺ and CD20⁺ Target Cells

The binding affinity of the CD79b binding arm of the CD79xCD20xCD3 trispecific molecules as well as control CD79bxCD3 and NullxCD20xCD3 were assessed using cell lines that were validated by flow cytometry to have different endogenous expression levels of CD79b and CD20 on the cell surface, shown in Table 9.

TABLE 9
CD79b and CD20 Antigen Density of B Lymphoma Cell Lines

| | | CD79b Antigen | CD20 Antigen |
|---|---|---|---|
| | | Density (Antigen | Density (Antigen |
| Cell Line | Cell Type | Number/cell) | Number/cell) |
| HBL-1 | Diffuse large B-cell | 429,649 | 73,467 |
| | lymphoma line | | |
| OCI-LY-10 | Diffuse large B-cell | 38,885 | 67,352 |
| | lymphoma line | | |
| CARNAVAL | Diffuse large B-cell | 98,176 | 118,789 |
| | lymphoma line | | |
| WILL2 | Diffuse large B-cell | 3,824 | 314 |
| | lymphoma line | | |

Diffuse large B-cell lymphoma cell lines were incubated for 1 hour with CD79bxCD20xCD3 test molecules C923B74, C923B99, and C923B38; CD79 CD3 test molecules 79C3B646, 79C3B651, and 79C3B601 and NullxCD20xCD3 control molecule C923B98 (1 μM starting concentration at 1:3 serial dilutions) at 37° C. All cells were washed with BD stain buffer (BD Biosciences; Cat #554657), centrifuged at 1200 RPM for 3 minutes, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:200 dilution along with Aqua Fixable Live/Dead stain (Invitrogen; Cat #L34957) at a 1:400 dilution. All cells were washed with BD stain buffer (BD Biosciences; Cat #554657), centrifuged at 1200 RPM for 3 mins, with supernatant discarded. Cells were analyzed using Intellicyt (Sartorius) flow cytometer and mean fluorescent intensity (MFI) was generated using Forcyt software (Sartorius). MFI was graphed and EC50 values generated using GraphPad PRISM v.8. Dose response curves were generated by transforming the x axis values using the formula x=lox. Data was then graphed using non-linear regression curve fit analysis “log(agonist) vs. response-variable slope (four parameter)”.

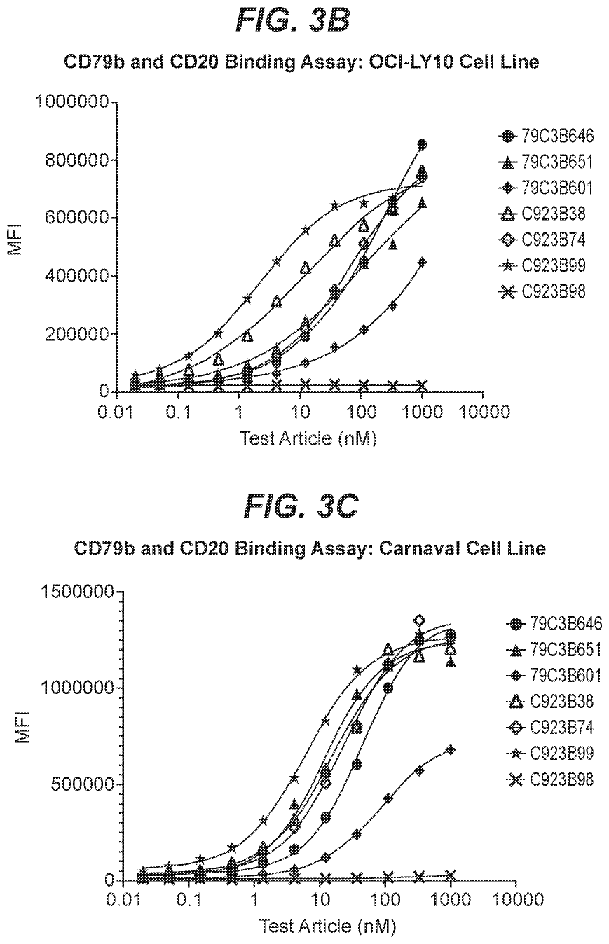

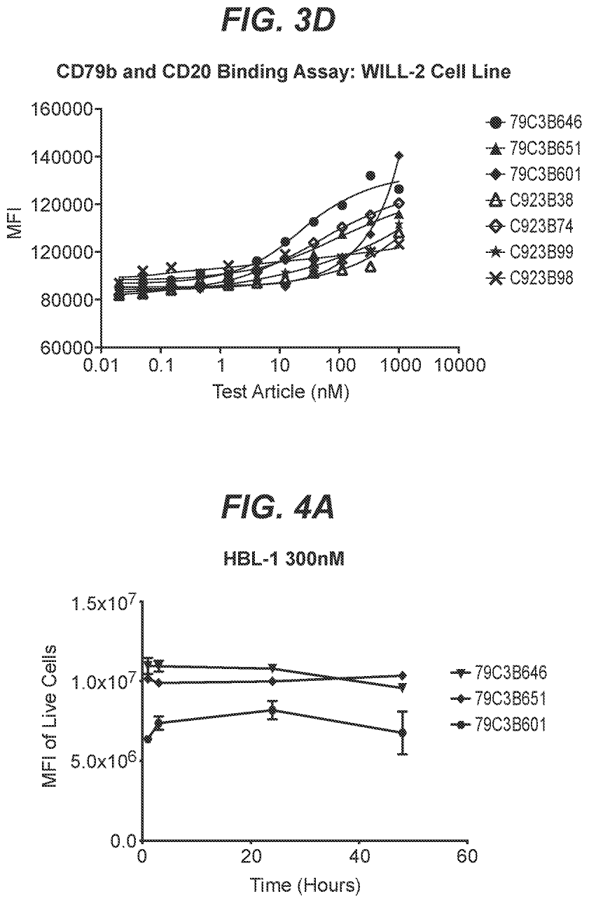

All CD79bxCD20xCD3 molecules showed good binding on cell lines expressing endogenous CD79b and CD20 on the cell surface, with some trispecific constructs showing better binding affinity across cell lines when compared to binding of CD79bxCD3 and CD20xCD3 control molecules, shown in FIGS. 3A-3D and Table 10. The CD79b binding arm of trispecific construct C923B99 showed the highest binding affinity across all tested cell lines, shown in FIGS. 3A-3D and Table 10.

TABLE 10
CD79bxCD20xCD3 Trispecific Cell Binding EC50 Values

| | HBL-1 | | Carnaval | WILL-2 |
|---|---|---|---|---|
| | EC50 | OCI-LY10 | EC50 | EC50 |
| | (nM) | EC50 (nM) | (nM) | (nM) |
| C923B38 | 43 | 12 | 16 | undetermined |
| C923B74 | 52 | 66 | 23 | undetermined |
| C923B99 | 8 | 2 | 6 | undetermined |
| 79C3B646 | 97 | undetermined | 44 | undetermined |
| 79C3B651 | 15 | undetermined | 12 | undetermined |
| 79C3B601 | 48 | undetermined | 89 | undetermined |
| C923B98 | undetermined | undetermined | Undetermined | undetermined |

Note:
“Undetermined” signifies that antibody binding did not reach the plateau and therefore EC50 value could not be determined.

### Kinetic Cell Binding of Bispecific CD79xCD3 Antibodies on CD79⁺ Target Cells

The binding kinetics of the CD79b binding arm of the CD79xCD3 bispecific molecules were assessed over a time course using cell lines that were validated by flow cytometry to have different endogenous expression levels of CD79b on the cell surface, shown in Table 11.

TABLE 11
CD79b Antigen Density of B Lymphoma Cell Lines

| | | CD79b Antigen Density |
|---|---|---|
| Cell Line | Cell Type | (Antigen Number/cell) |
| HBL-1 | Diffuse large B-cell lymphoma line | 429,649 |
| OCI-LY10 | Diffuse large B-cell lymphoma line | 38,885 |
| CARNAVAL | Diffuse large B-cell lymphoma line | 98,176 |

Diffuse large B-cell lymphoma cell lines were incubated for 1, 3, 24, and 48 hours with CD79b xCD3 test molecules 79C3B646, 79C3B651, and 79C3B601 (300 nM, 60 nM, 12 nM) at 37° C. At each time point, cells were washed with BD stain buffer (BD Biosciences; Cat #554657), centrifuged at 1200 RPM for 3 mins, with supernatant discarded. Cells were then stained for 30 minutes at 4° C. with BD stain buffer containing AlexaFluor 647 labeled anti-human IgG secondary antibody (Jackson Immuno; Cat #109-606-098) at a 1:200 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed and EC50 values generated using GraphPad PRISM v.8.

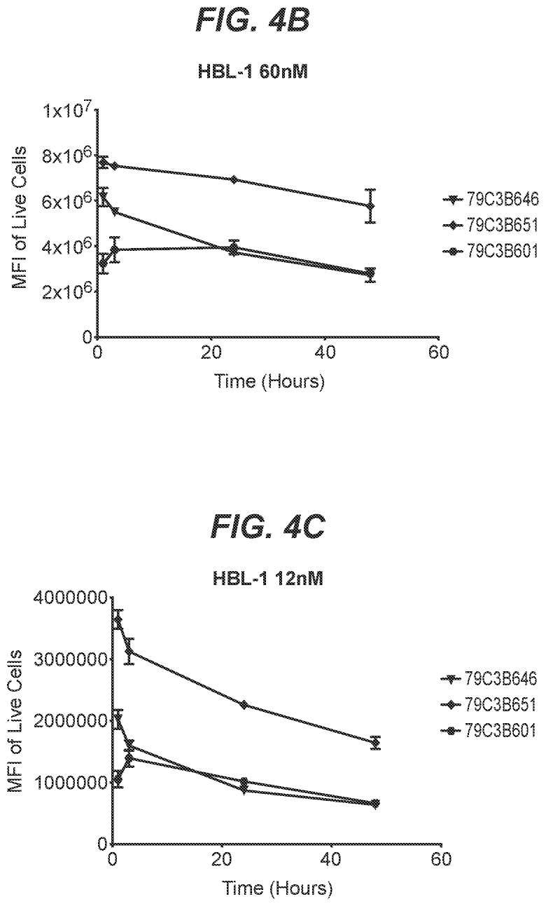

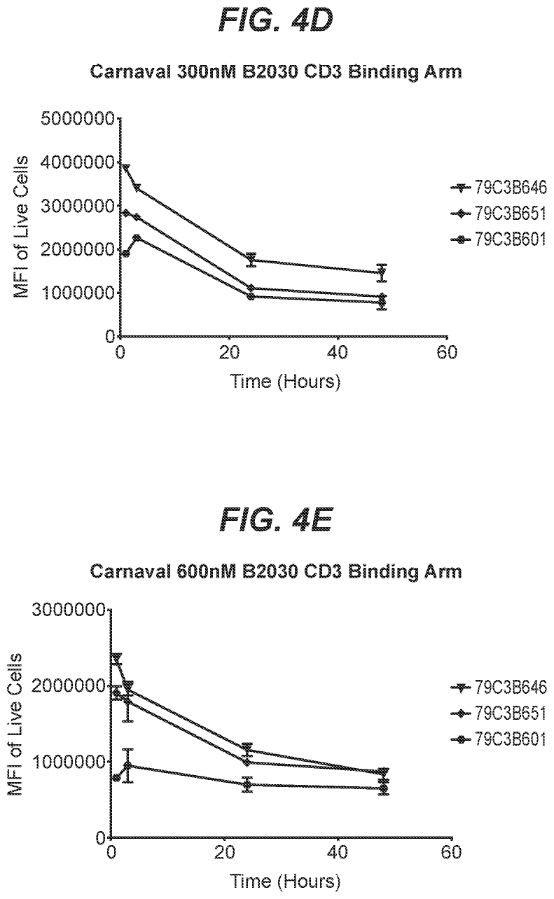

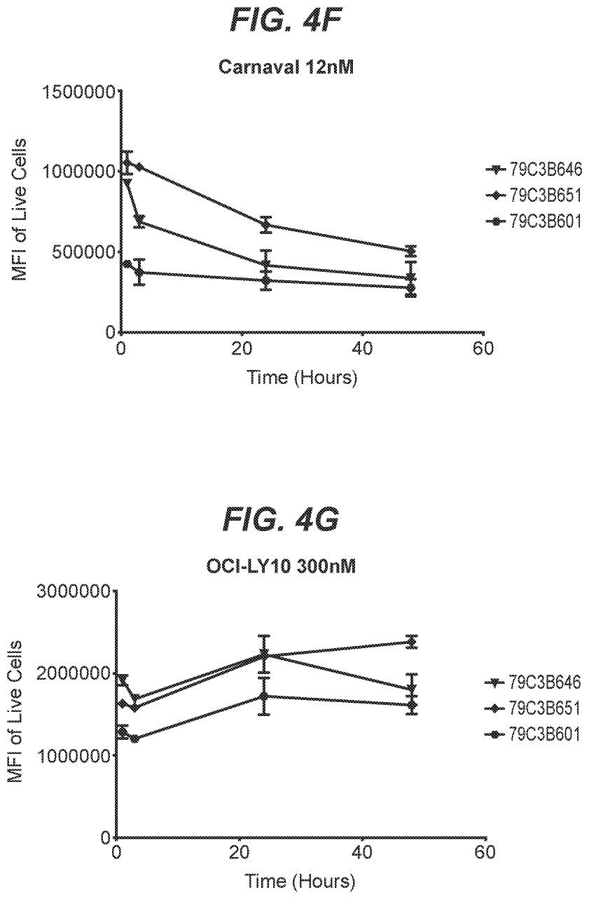

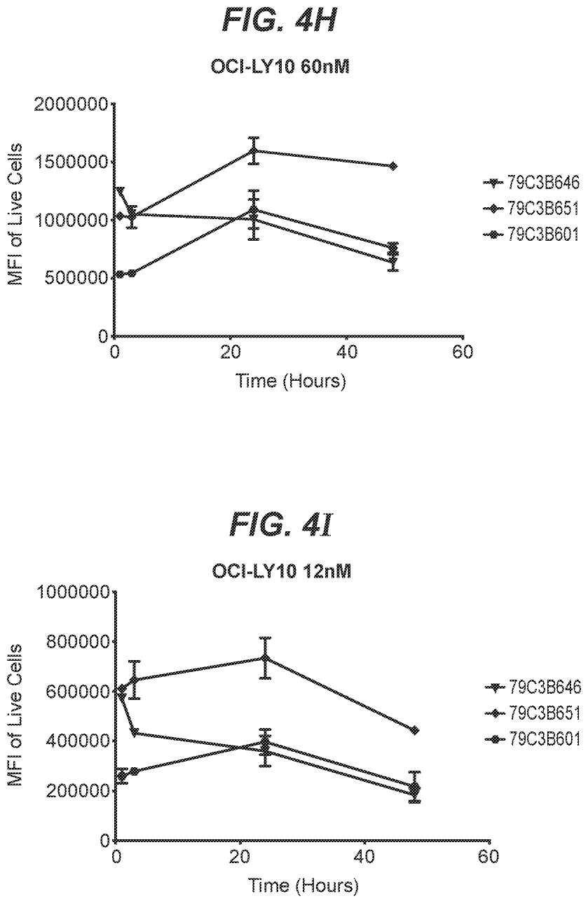

All CD79bxCD3 bispecific constructs showed steady CD79b binding kinetics with minimal loss of signal over time, as shown in FIGS. 4A-4I. 79C3B651 showed superior binding kinetics and the least amount of signal loss over time, shown in FIGS. 4A-4I.

Kinetic Cell Binding of Trispecific CD79xCD20xCD3 Antibodies on CD79b⁺ and CD20⁺ Target Cells

The binding kinetics of the CD79b and CD20 binding arms of the CD79xCD20xCD3 trispecific molecules were assessed over a time course using cell lines that were validated by flow cytometry to have different endogenous expression levels of CD79b and CD20 on the cell surface, shown in Table 12.

TABLE 12
CD79b and CD20 Antigen Density of B Lymphoma Cell Lines

| | | CD79b Antigen | CD20 Antigen |
|---|---|---|---|
| | | Density (Antigen | Density (Antigen |
| Cell Line | Cell Type | Number/cell) | Number/cell) |
| HBL-1 | Diffuse large B-cell | 429,649 | 73,467 |
| | lymphoma line | | |
| OCI-LY-10 | Diffuse large B-cell | 38,885 | 67,352 |
| | lymphoma line | | |
| CARNAVAL | Diffuse large B-cell | 98,176 | 118,789 |
| | lymphoma line | | |

Diffuse large B-cell lymphoma cell lines were incubated for 1, 3, 24, and 48 hours with CD79bxCD20xCD3 test molecules C923B74, C923B99, and C923B38; CD79xCD3 test molecules 79C3B646, 79C3B651, and 79C3B601 and NullxCD20xCD3 control molecule C923B98 (300 nM, 60 nM, 12 nM) at 37° C. At each time point, cells were washed with BD stain buffer (BD Biosciences; Cat #554657), centrifuged at 1200 RPM for 3 mins, with supernatant discarded. Cells were then stained for 30 minutes at 4° C. with BD stain buffer containing AlexaFluor 647 labeled anti-human IgG secondary antibody (Jackson Immuno; Cat #109-606-098) at a 1:200 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed and EC50 values generated using GraphPad PRISM v.8.

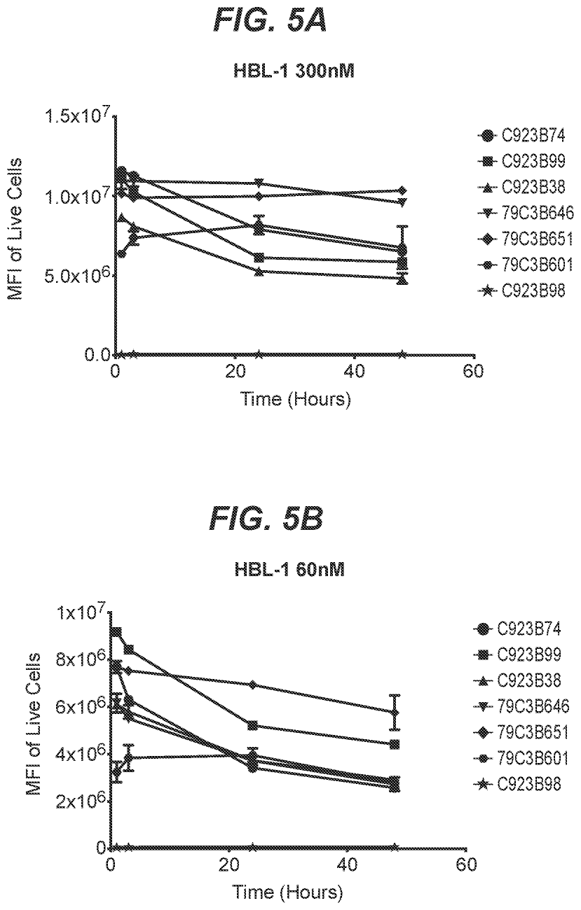

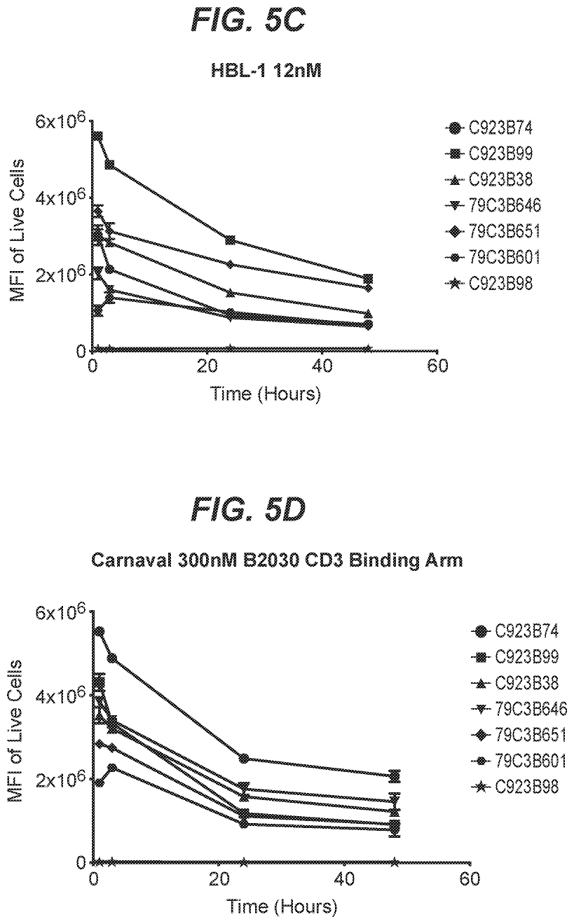

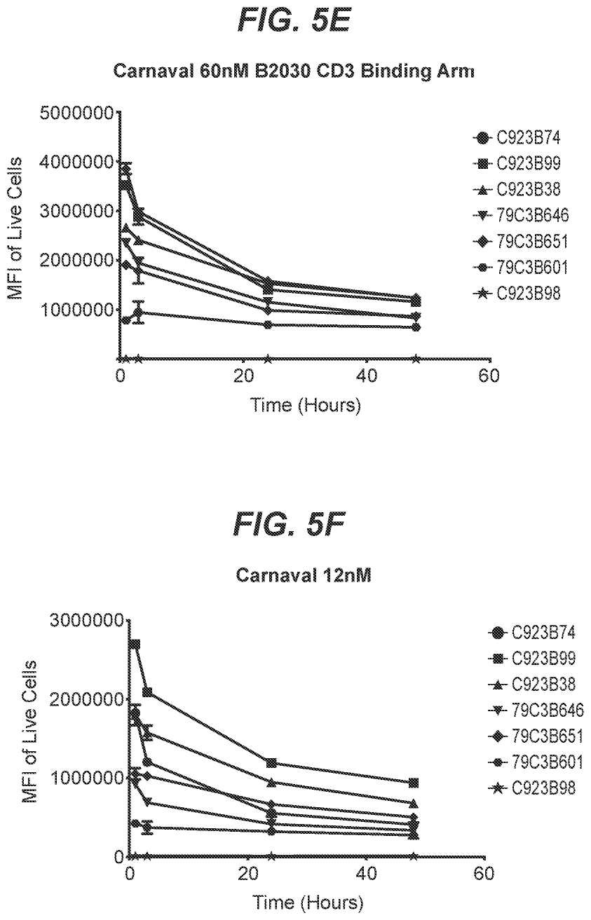

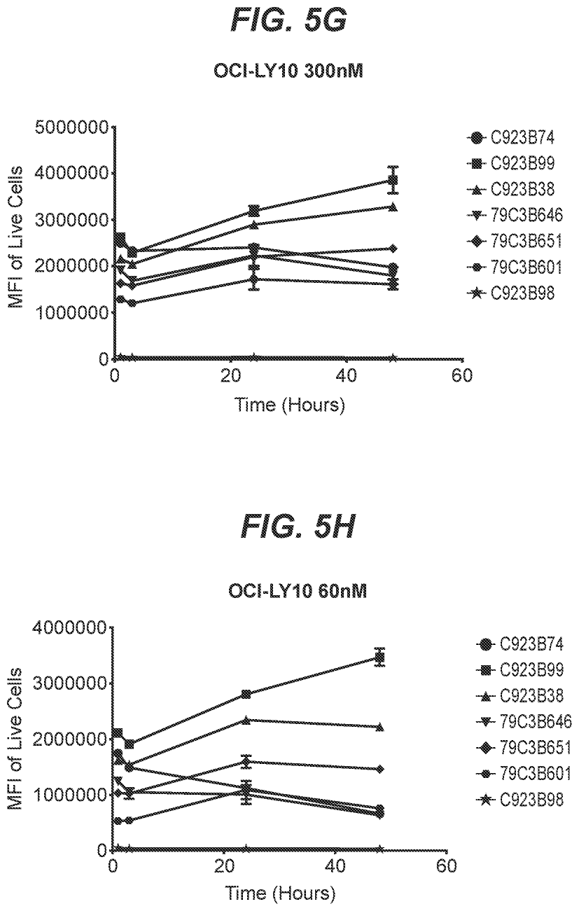

All CD79bxCD20xCD3 bispecific constructs showed steady CD79b binding kinetics with minimal loss of signal over time, shown in FIGS. 5A-5I. Trispecific construct C923B99 and bispecific construct 79C3B651, which both have the same CD79b and CD20 binding arms, showed superior binding kinetics and the least amount of signal loss over time, shown in FIGS. 5A-5I.

### Binding of Bispecific CD79xCD3 Antibodies and Trispecific CD79xCD20xCD3 Antibodies on Pan T-Cells

Binding of the CD3 arm of CD79xCD3 bispecific and CD79bxCD20xCD3 trispecific constructs was assessed using cryo-preserved, negatively selected, primary human CD3⁺ pan T cells. Primary human CD3⁺ pan T cells from four different donors were incubated for 1 hour with CD79bxCD20 x CD3 test molecules C923B74, C923B99, and C923B38 or CD79xCD3 test molecules 79C3B646, 79C3B651 (1 uM starting concentration at 1:3 serial dilutions) at 37° C. All cells were washed with BD stain buffer (BD Biosciences; Cat #554657), centrifuged at 1200 RPM for 3 mins, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:300 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed using GraphPad PRISM v.8. Dose response curves were generated by transforming the x axis values using the formula x=lox. Data was then graphed using non-linear regression curve fit analysis “log(agonist) vs. response-variable slope (four parameter)”.

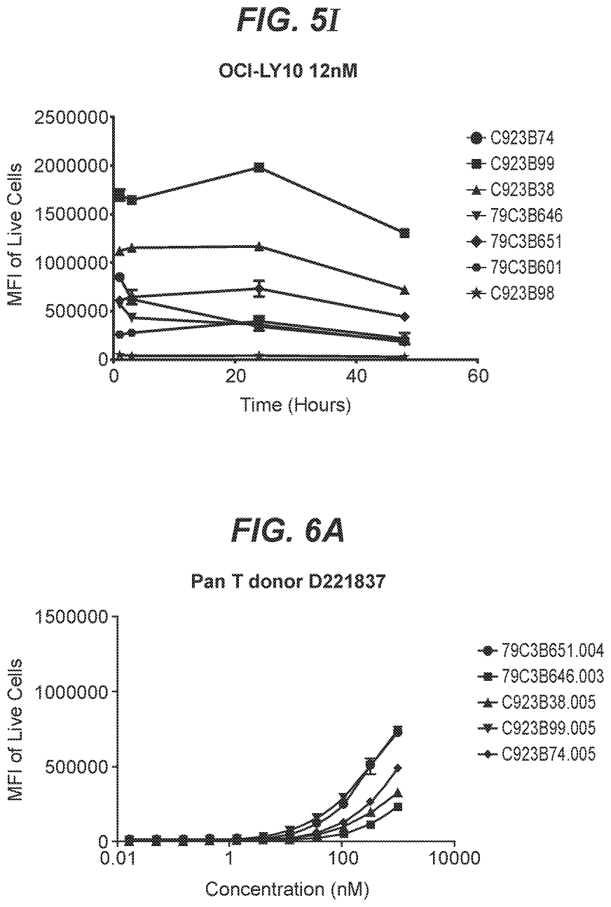

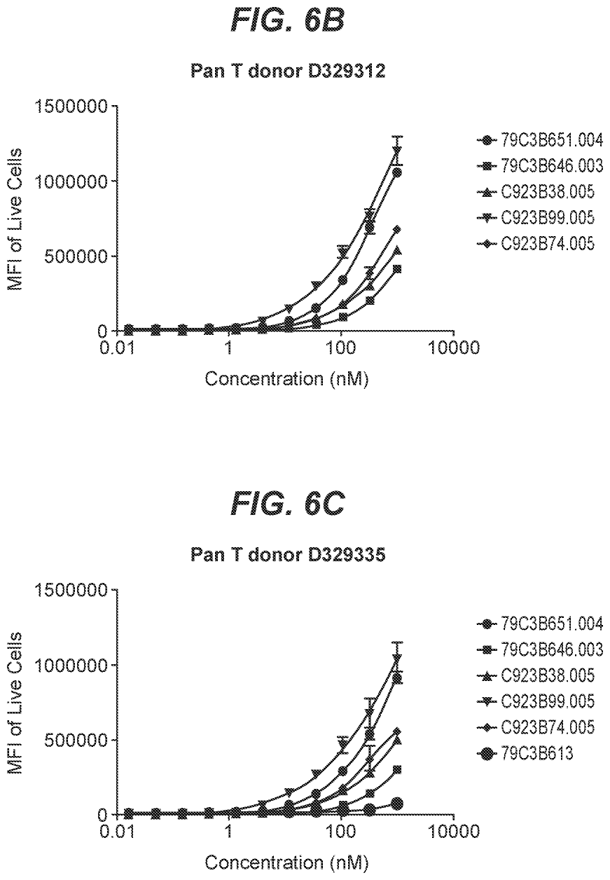

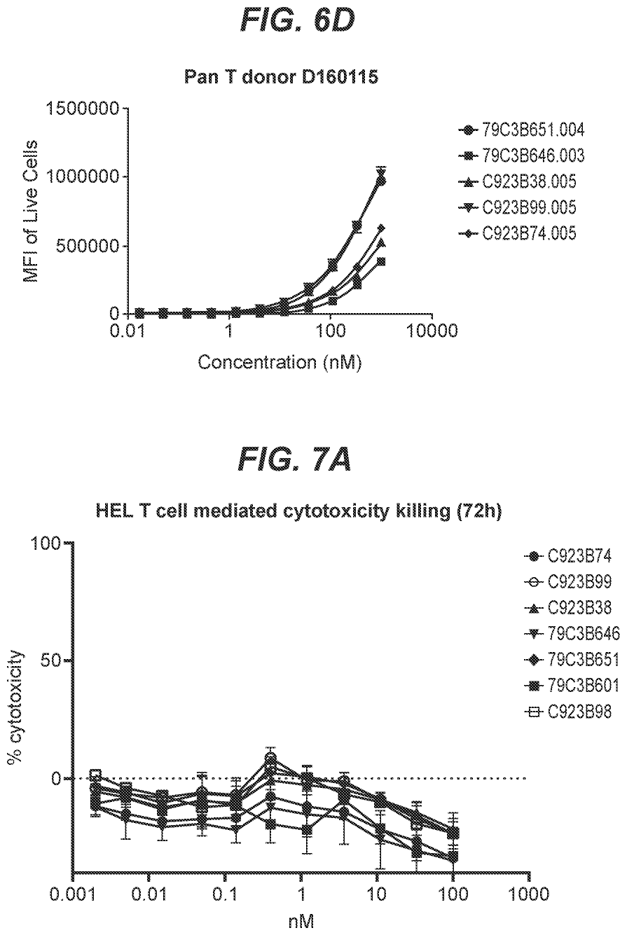

All CD79bxCD20 x CD3 and CD79bxCD3 molecules showed moderate binding on all donor Pan T cells expressing endogenous CD3 on the cell surface, shown in FIGS. 6A-6D.

### Example 5: Functional Characterization: Antagonistic Activity of CD79xCD3 Bispecific and CD79xCD20xCD3 Trispecific Antibodies

Bispecific CD79xCD3 and Trispecific CD79xCD20xCD3 Mediated Cytotoxicity Against CD79B⁺ and CD79B⁻ Target Cells

mKATE2 DLBCL target cells were maintained in complete RPMI (ThermoFisher, catalog #11875093)1640 media containing 10% heat inactivated fetal bovine serum. Prior to the assay, antibodies were made at 3-fold serial dilutions in the at RPMI 1640 media containing 10% heat inactivated fetal bovine serum, at 4-fold expected final concentration. A volume of 50 μL of medium-diluted bsAb or trispecific Ab in each well of a 96-well plate were further diluted into 200 μL by adding a mix of target and effector cell suspension. The target cell lines were harvested by centrifuge at 400×g for 5 min, washed one time with phenol red-free RPMI 1640 media, counted and suspended in fresh complete phenol red-free RPMI 1640 media at 1×10⁶ cells/mL. Healthy donor T cells (isolated by CD3-negative selection provided by Discovery Life Sciences) were thawed in complete phenol red-free media (RPMI 1640 media containing 10% heat inactivated fetal bovine serum), counted and suspended in fresh complete phenol red-free RPMI 1640 media at 1×10⁶ cells/mL. Target cells and T cells were mixed to obtain 5:1 effector to target cell ratio. Cell suspension was added to antibody dilution wells according to plate layout (150 μL/well).

After mixing target and T cells with corresponding bsAb dilution, 80 μL from each well, containing 200 μl with 10000 target and 50000 T cells, were dispensed in a 384 well plate, in duplicate. Plates were sealed using a Breathe-Easy membrane seal. Next, cocultures were placed in an IncuCyte ZOOM live-content imaging system, and images were automatically acquired in both phase and fluorescence channels every 6 hours for 3 to 6 days with a 4× objective lens (single whole well image). IncuCyte Zoom software was used to detect target cells based on mKATE2 expression using optimized process definition parameters. To measure the amount of target cells/well, the total red area was quantified, and raw values were exported in Excel (Microsoft Office). To quantify cancer cell killing over time, the average values for each replicate were pasted in Prism (GraphPad; version 7 for PC). Expansion indexes (EI) per timepoint were calculated by dividing value at Tx by TO. Growth inhibition (GI) was calculated by normalizing each timepoint to the value of the untreated well average at that timepoint. From the GI values, area under the curve (AUC) values were derived for each condition. After normalizing the AUC to the untreated control (target with effector), antibody concentrations were plotted against the AUC values as a dose response. EC50 values were generated using GraphPad PRISM v.8. Dose response curves were generated by transforming the x axis values using the formula x=lox. Data was then graphed using non-linear regression curve fit analysis “log(agonist) vs. response-variable slope (four parameter)”. CD79bxCD3 bispecific antibodies (79C3B645, 79C3B646, 79C3B601, 79C3B605, 79C3B650, 79C3B651) and CD79bxCD20xCD3 trispecific antibodies (C923B73, C923B74, C923B36, C923B38, C923B99, C923B95) were evaluated for cytotoxicity on HBL1 and OCI-Ly10 cells. IC50 (pM) values are listed in Table 13, Table 14, Table 15, and Table 16.

TABLE 13
HBL-1 killing Incucyte (Average of 2 independent experiments)

| Protein ID | CD79b | CD20 | CD3 | IC50 (pM) |
|---|---|---|---|---|
| 79C3B645 | CD9B330 | NA | CD3B2089 | 7189.0 |
| 79C3B646 | CD9B330 | NA | CD3B2030 | 257.4 |
| C923B73 | CD9B330 | C20B22 | CD3B2089 | 6805.0 |
| C923B74 | CD9B330 | C20B22 | CD3B2030 | 346.3 |
| 79C3B605 | CD9B374 | NA | CD3B2089 | 29549.0 |
| 79C3B601 | CD9B374 | NA | CD3B2030 | 203.9 |
| C923B36 | CD9B374 | C20B22 | CD3B2089 | 31040.0 |
| C923B38 | CD9B374 | C20B22 | CD3B2030 | 301.2 |
| 79C3B650 | CD9B643 | NA | CD3B2089 | 43314.0 |
| 79C3B651 | CD9B643 | NA | CD3B2030 | 32.5 |
| C923B95 | CD9B643 | C20B22 | CD3B2089 | 4891.0 |
| C923B99 | CD9B643 | C20B22 | CD3B2030 | 69.2 |

NA = Not Applicable

TABLE 14
OCI-Ly10 killing Incucyte (Average of 2 independent experiments)

| Protein ID | CD79b | CD20 | CD3 | IC50 (nM) |
|---|---|---|---|---|
| 79C3B645 | CD9B330 | NA | CD3B2089 | 18.0 |
| 79C3B646 | CD9B330 | NA | CD3B2030 | 18.3 |
| C923B73 | CD9B330 | C20B22 | CD3B2089 | 132.4 |
| C923B74 | CD9B330 | C20B22 | CD3B2030 | 25.6 |
| 79C3B605 | CD9B374 | NA | CD3B2089 | 54.3 |
| 79C3B601 | CD9B374 | NA | CD3B2030 | 11.7 |
| C923B36 | CD9B374 | C20B22 | CD3B2089 | 42.0 |
| C923B38 | CD9B374 | C20B22 | CD3B2030 | 8.0 |
| 79C3B650 | CD9B643 | NA | CD3B2089 | 7.0 |
| 79C3B651 | CD9B643 | NA | CD3B2030 | 4.7 |
| C923B95 | CD9B643 | C20B22 | CD3B2089 | 14.8 |
| C923B99 | CD9B643 | C20B22 | CD3B2030 | 5.6 |

NA = Not Applicable

TABLE 15
CARNAVAL killing (Incucyte)

| Protein ID | CD79b | CD20 | CD3 | IC50 (nM) |
|---|---|---|---|---|
| 79C3B646 | CD9B330 | NA | CD3B2030 | 1.393 |
| C923B74 | CD9B330 | C20B22 | CD3B2030 | 0.741 |
| 79C3B601 | CD9B374 | NA | CD3B2030 | 1.645 |
| C923B38 | CD9B374 | C20B22 | CD3B2030 | 0.465 |
| C923B99 | CD9B643 | C20B22 | CD3B2030 | 0.285 |

TABLE 16
Daudi killing (Incucyte)

| Protein ID | CD79b | CD20 | CD3 | IC50 (nM) |
|---|---|---|---|---|
| 79C3B646 | CD9B330 | NA | CD3B2030 | 0.597 |
| C923B74 | CD9B330 | C20B22 | CD3B2030 | 0.100 |
| 79C3B601 | CD9B374 | NA | CD3B2030 | 0.406 |
| C923B38 | CD9B374 | C20B22 | CD3B2030 | 0.071 |
| C923B99 | CD9B643 | C20B22 | CD3B2030 | <Conc tested |

NA = Not Applicable

### FACS T Cell Killing Data on Panel of Target Positive (CD79b+ and CD2+) and Target Negative (CD79B− and CD20−) Cell Lines

Functional activity of the CD79bxCD3 bispecific and CD79bxCD20xCD3 trispecific constructs was assessed at 72 hr time point in an in vitro T cell killing assay by flow cytometry using cell lines that were validated by flow cytometry to have different endogenous expression levels of CD79b and CD20 on the cell surface, shown in Table 17.

TABLE 17
CD79b and CD20 Antigen Density of B Lymphoma Cell Lines

| | | | CD20 Antigen |
|---|---|---|---|
| | | CD79b Antigen | Density |
| | | Density (Antigen | (Antigen |
| Cell Line | Cell Type | Number/cell) | Number/cell) |
| HBL-1 | Diffuse large B-cell | 429,649 | 73,467 |
| | lymphoma line | | |
| OCI-LY10 | Diffuse large B-cell | 38,885 | 67,352 |
| | lymphoma line | | |
| CARNAVAL | Diffuse large B-cell | 98,176 | 118,789 |
| | lymphoma line | | |
| K562 | Chronic myelogenous | 0 | 0 |
| | leukemia | | |
| HEL | Erythroleukemia | 0 | 0 |

Target cancer cells were maintained in complete RPMI 1640 (ThermoFisher, catalog #11875093) media containing 10% heat inactivated fetal bovine serum. Prior to the assay, antibodies were made at 3-fold serial dilutions in RPMI 1640 media containing 10% heat inactivated fetal bovine serum, at 4-fold expected final concentration. A volume of 50 μL of medium-diluted bispecific or trispecific Ab in each well of a 96-well plate were further diluted into 200 μL by adding a mix of target and effector cell suspension. The target cell lines were harvested by centrifuge at 400×g for 5 min, washed one time with RPMI 1640 media. Target cancer cells were stain targets with CellTrace CFSE (ThermoFisher; Cat #: C34554) diluted 1/5000. Healthy donor T cells (isolated by CD3-negative selection provided by Discovery Life Sciences) were thawed in complete media (RPMI 1640 media containing 10% heat inactivated fetal bovine serum), counted and suspended in fresh complete phenol red-free RPMI 1640 media at 1×10⁶ cells/mL. Target cells and T cells were mixed to obtain 5:1 effector to target cell ratio. Cell suspension was added to antibody dilution wells according to plate layout (150 μL/well). Cells were incubated for 72 hours with CD79bxCD3 or CD79bxCD20xCD3 test molecules (100 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 15 minutes at room temperature with Fixable Live/Dead stain (ThermoFisher; Cat #65-0865-14) at a 1:1000 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 18), antibodies amount added as listed in the table. All cells were washed with BD stain buffer (BD Biosciences; Cat #554657), centrifuged at 1200 RPM for 3 mins, with supernatant discarded. Cells were analyzed using FACS Lyric (BD) flow cytometer and percent of cancer cell killing was generated using Cytobank. Percent of cancer cell killing was graphed and IC50 values generated using GraphPad PRISM v.8. Dose response curves were generated by transforming the x axis values using the formula x=lox. Data was then graphed using non-linear regression curve fit analysis “log(inhibitor) vs. response-variable slope (four parameter)”.

TABLE 18
Flow Panel Antibodies for T cell killing Assay

| | | | | | Amount |
|---|---|---|---|---|---|
| Antibody | Conjugated | | Catalog | LOT | added per |
| Name | Fluorophore | Vendor | Number | Number: | well (μl) |
| CD4 | V500 | BD Biosciences | 560768 | 9340575 | 2μ/well |
| CD8 | PerCPCy5.5 | BD Biosciences | 560662 | 9290508 | 2 μl/well |
| CD69 | PE | BD Biosciences | 560968 | 9049603 | 10 μl/well |
| CD25 | BV421 | BD Biosciences | 562443 | 10302 | 2 μl/well |

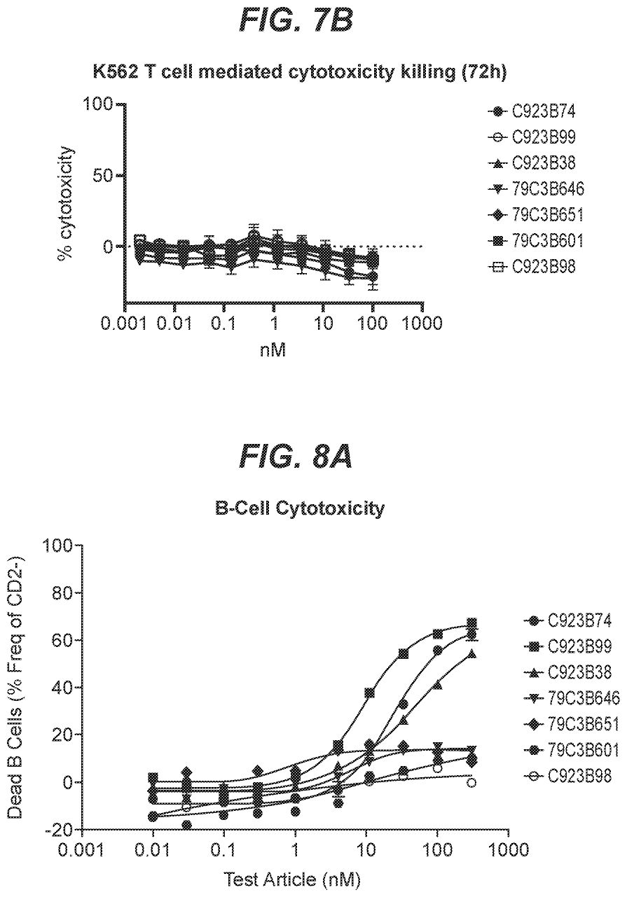

CD79b × CD20 × CD3 trispecific mediated more potent cytotoxicity as compared to bispecific constructs in CD79b− and CD20− target positive cell lines. IC50 (pM) values are listed in Table 19. No killing has been observed in target negative cell lines (FIG. 7A-7B).

TABLE 19
Killing of target positive (CARNAVAL, OCI-Ly10) cell lines (FACS).

| | | | | | HBL-1 | OCI-LY10 |
|---|---|---|---|---|---|---|
| | | | | CARNAVAL | IC50 | IC50 |
| Protein ID | CD79b | CD20 | CD3 | IC50 (nM)* | (nM) | (nM)** |
| 79C3B646 | CD9B330 | NA | CD3B2030 | 0.29 | 0.73 | >100 nM |
| C923B74 | CD9B330 | C20B22 | CD3B2030 | 0.35 | 2.42 | 24.19 |
| 79C3B601 | CD9B374 | NA | CD3B2030 | NA | 2.86 | >100 nM |
| C923B38 | CD9B374 | C20B22 | CD3B2030 | 0.33 | 2.71 | 48.59 |
| 79C3B651 | CD9B643 | NA | CD3B2030 | 0.25 | 2.20 | >100 nM |
| C923B99 | CD9B643 | C20B22 | CD3B2030 | 0.17 | 1.68 | 16.95 |

NA = not applicable
*average values of T cell mediated killing from 3 independent T cell donors
**average values of T cell mediated killing from 4 independent T cell donors

### Bispecific CD79bxCD3 Mediated Cytotoxicity Against Autologous B-Cells

Functional activity of the CD79bxCD3 bispecific constructs was assessed in an in vitro autologous B cell depletion assay. This functional assay utilizes PBMCs to focus on the killing of primary B cells as well as T cell activation on donor matched primary cells. Cryo-preserved PBMCs from 3 different human donors were incubated for 72 hours with CD79bxCD3 test molecules 79C3B646, 79C3B651, and 79C3B601 (300 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 10 minutes at room temperature with BD stain buffer containing Fc blocking agent (Accurate Chemical and Scientific Corp; Cat #NB309) and Near IR Fixable Live/Dead stain (Invitrogen; Cat #L10119) at a 1:400 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 20) at a 1:100 dilution. All cells were washed with BD stain buffer (BD Biosciences; Cat #554657), centrifuged at 1200 RPM for 3 mins, with supernatant discarded. Cells were analyzed using Intellicyt (Sartorius) flow cytometer and mean fluorescent intensity (MFI) was generated using Forcyt software (Sartorius). MFI was graphed and EC50 values generated using GraphPad PRISM v.8. Dose response curves were generated by transforming the x axis values using the formula x=lox. Data was then graphed using non-linear regression curve fit analysis “log(agonist) vs. response-variable slope (four parameter)”.

TABLE 20
Flow Panel Antibodies for Autologous B Cell Depletion Assay

| | Conjugated | | Catalog |
|---|---|---|---|
| Antibody Name | Fluorophore | Vendor | Number |
| Anti-human CD25 | BV650 | BD Biosciences | 563719 |
| Anti-Human CD4 | BV510 | Biolegend | 317444 |
| Anti-Human CD8 | PE-Cy7 | Biolegend | 301012 |
| Anti-Human CD20 | PE | Biolegend | 302306 |
| Anti-Human CD11c | AF647 | BD Biosciences | 565911 |
| Anti-Human CD2 | BV605 | BD Biosciences | 740391 |

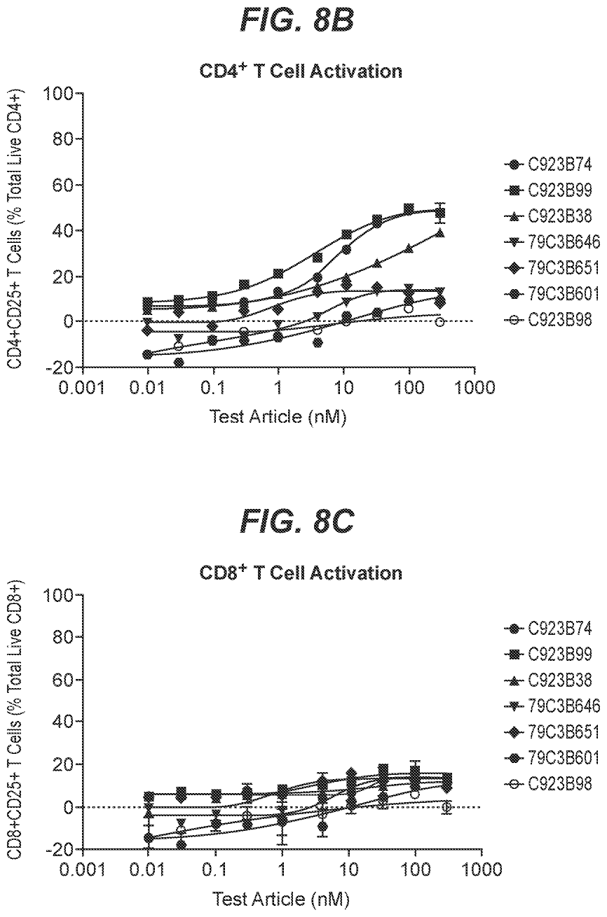

CD79bxCD3 bispecific constructs showed a maximum drug mediated cytotoxicity of 20 percent with low levels of CD4⁺ and CD8⁺ T cell activation as demonstrated by CD25 expression on these T cell subsets, as shown in FIGS. 8A-8C. The CD79bxCD20xCD3 trispecific has a synergistic effect on drug mediated cytotoxicity when compared to control molecules as shown in Table 21.

TABLE 21
CD79b × CD20 × CD3 EC50 Values and Maximum Cytotoxicity

| | | | | Donor 1 | | Donor 2 | | Donor 3 | |
|---|---|---|---|---|---|---|---|---|---|
| Construct | | | | EC50 | | EC50 | | EC50 | |
| Name | CD79b Arm | CD20 Arm | CD3 Arm | (nM) | Cₘₐₓ | (nM) | Cₘₐₓ | (nM) | Cₘₐₓ |
| C323874 | 8330-Fab | C20822 scFv | CD382030 | 78 | 34% | 23 | 66% | UD* | 40% |
| 78C38846 | | N/A | CD382030 | 40 | 9% | 6 | 14% | 8 | 35% |
| CZ3898 | N/A | C20822 scFv | CD382030 | UD* | 11% | UD* | 5% | 88 | 52% |
| C923899 | 8543-Fab | C20822 scFv | CD382030 | UD* | 35% | 9 | 67% | 23 | 66% |
| 79C38651 | | N/A | CD982030 | UD* | 5% | UD* | 13% | 17 | 34% |
| C23888 | N/A | C20822 scFv | CD382030 | UD* | 11% | UD* | 5% | 88 | 52% |
| C923836 | 8374-Fab | C20822 scFv | CD382030 | 1 | 24% | 50 | 60% | UD* | 50% |
| 79038601 | | N/A | CD382030 | UD* | 10% | UD* | 10% | 20 | 32% |
| C23888 | N/A | C20822 scFv | CD382030 | UD* | 11% | UD* | 5% | 88 | 52% |

*Undetermined

### Example 6: Biophysical Characterization

### Binding Affinity by SPR

General Protocol for SPR Affinity Assessment: Affinity assessment of the bispecific trispecific constructs against human CD79b were measure using recombinantly expressed extracellular domain of CD79b short and long isoforms (CD9W7.001 and CD9W8.001, respectively) by Surface Plasmon Resonance (SPR) using a Biacore 8k SPR system (Biacore) at 25° C. in HBSP+ buffer. Cross-reactivity of the same antibody panel was also assessed against cyno and mouse antigens (CD9W1.001 and CD9W105.001, respectively). Briefly, a C1 sensor chip was immobilized with anti-human Fc (target immobilization levels of >400 RU) using vendor recommended amino coupling protocol. The test antibodies were captured through immobilized anti-Fc and was followed by the injection of different CD79b constructs at different concentration series (human CD79b short and long isoforms: 30 nM-0.37 nM at 3-fold dilutions; cyno and mouse CD79b: 3000 nM-37 nM at 3-fold dilutions). The association and dissociation phases were measured for 2 or 3 minutes and 30 minutes, respectively. Binding of the trispecifics (C923B168 and C923B169) to CD3 was tested by injecting CD3W220.001 at 100 nM-1.23 nM at 3-fold dilutions, with association and dissociation phases were measured for 3 min and 15 min, respectively (CD79b-00478).

The raw binding sensorgrams were processed using Biacore Insight software (Biacore) by double-referencing and the processed sensorgrams were analyzed for cross-reactivity and fitted to a 1:1 Langmuir model to obtain on-rates, off-rates and affinities.

SPR Binding Results: As shown in Table 22 and Table 23, the bispecific and trispecific antibodies bound to the human CD79b long isoform (hu CD79b long) with affinities from 0.02-0.06 nM, and to the CD79b short isoform (hu CD79b short) with affinities between 0.27-0.64 nM. The antibody panel showed very poor cross-reactivity to cyno CD79b (KD estimated >3000 nM) or did not bind to mouse CD79b. C923B168 binds recombinant CD3 antigen with an affinity of 0.5 nM. No quantitative kinetics/affinities were reported for those with complex kinetic binding profiles using the specified antigens, as noted in the summary tables below.

TABLE 22
Binding affinities for bispecific antibody constructs

| | KD to hu | KD to hu | |
|---|---|---|---|
| | CD79b long | CD79b | KD to hu |
| Name | (M) | short (M) | CD3 (M) |
| 79C3B601 | 4.6E−11 | 5.6E−10 | n.d** |
| 79C3B646 | 2.2E−11 | 5.8E−10 | n.d** |
| 79C3B651 | 5.2E−11 | 3.5E−10 | n.d** |
| 79C3B605 | n.d* | n.d* | n.d** |
| 79C3B645 | n.d* | n.d* | n.d** |
| 79C3B650 | n.d* | n.d* | n.d** |

*samples not submitted for SPR binding analysis
**Affinities for CD3 not determined due to complex SPR binding profiles observed for Cris7b derived CD3 antibodies (historically observed results).

TABLE 23
Binding affinities for trispecific antibody constructs

| | KD to hu | KD to hu | | |
|---|---|---|---|---|
| | CD79b long | CD79b | KD to hu | KD to hu |
| Name | (M) | short (M) | CD20 (M) | CD3 (M) |
| C923B38 | 6.5E−11 | 6.4E−10 | n.d** | n.d** |
| C923B74 | 2.3E−11 | 3.9E−10 | n.d** | n.d** |
| C923B99 | 4.0E−11 | 2.7E−10 | n.d** | n.d** |
| C923B36 | n.d* | n.d* | n.d** | n.d** |
| C923B73 | n.d* | n.d* | n.d** | n.d** |
| C923B95 | n.d* | n.d* | n.d** | n.d** |
| C923B168 | 1.92E−10 | n.d* | n.d** | 4.96E−10 |
| C923B169 | 1.64E−10 | n.d* | n.d** | n.d** |

*samples not submitted for SPR binding analysis
**Affinities for CD20 or CD3 not determined due to SPR constraints with CD20 nanodiscs or complex binding profiles observed for Cris7b derived CD3 antibodies (historically observed results)

### Binding Epitope by HDX-MS

The CD79b epitopes bound by trispecific molecules CD9B374 and CD9B643 were mapped by Hydrogen Deuterium Exchange Mass Spectrometry (HDX-MS) according to the following protocol.

General Procedure for HDX-MS Data Acquisition. HDX-MS sample preparation was performed with automated HDx system (LEAP Technologies, Morrisville, NC). The columns and pump were: protease, protease type XIII (protease from Aspergillus saitoi, type XIII)/pepsin column (w/w, 1:1; 2.1×30 mm) (NovaBioAssays Inc., Woburn, MA); trap, ACQUITY UPLC BEH C18 VanGuard Pre-column (2.1×5 mm) (Waters, Milford, MA), analytical, Accucore C18 (2.1×100 mm) (Thermo Fisher Scientific, Waltham, MA); and LC pump, VH-P10-A (Thermo Fisher Scientific). The loading pump (from the protease column to the trap column) was set at 600 μL/min with 0.1% aqueous formic acid. The gradient pump (from the trap column to the analytical column) was set from 9% to 35% acetonitrile in 0.1% aqueous formic acid in 20 min at 100 μL/min.

MS Data Acquisition. Mass spectrometric analyses were carried out using an LTQ™ Orbitrap Fusion Lumos mass spectrometer (Thermo Fisher Scientific) with the capillary temperature at 275° C., resolution 120,000, and mass range (m/z) 300-1,800.

HDX-MS Data Extraction. BioPharma Finder 3.0 (Thermo Fisher Scientific) was used for the peptide identification of non-deuterated samples prior to the HDX experiments. HDExaminer version 2.5 (Sierra Analytics, Modesto, CA) was used to extract centroid values from the MS raw data files for the HDX experiments.

HDX-MS Data Analysis. The extracted HDX-MS data were further analyzed in Excel. All exchange time points (at pH 6.4 or pH 7.4 at 3.2° C.) were converted to the equivalent time points at pH 7.4 and 23° C.

### Results

HDX-MS analysis of CD9B374 and CD9B643 indicate binding to a nearly identical, conformational epitope of CD79 made up of residues 30-42 (SEDRYRNPKGSAC; SEQ ID NO: 253), 50-52 (PRF), 81-86 (EMENP; SEQ ID NO: 254), and 144-148 (GFSTL; SEQ ID NO: 255). The residue numbers are those of CD79B_Human (P40259).

### Thermal Stability of Trispecific CD79bxCD20xCD3 Antibodies by DSC and DSF

The thermal stability of C923B168 and C923B169 was determined by Differential Scanning Calorimetry (DSC) and differential scanning fluorimetry (DSF).

In this characterization, Tonset and Tagg were determined by DSF and the other thermal stability transitions of Tms were determined by DSC. As shown in Table 24, C923B168 and C923B169 have good thermal stability with Tonset >61° C. and Tm1>65°

TABLE 24
Transition temperatures for trispecific CD79b × CD20 × CD3 antibodies

| | Tonset | | Tm1 | | Tm2 | | Tm3 | | Tagg | |
|---|---|---|---|---|---|---|---|---|---|---|
| Sample ID | ° C. | σ | ° C. | σ | ° C. | σ | ° C. | σ | ° C. | σ |
| C923B168.008 | 61.3 | 0.15 | 65.5 | 0.03 | 73.5 | 0.18 | 77.3 | 0.07 | 73.8 | 0.37 |
| C923B169.008 | 61.7 | 0.07 | 68.4 | 0.03 | 75.1 | 0.44 | 77.7 | 0.21 | 74.2 | 0.5 |

### Example 7: Functional Characterization of CD79xCD20xCD3 Trispecific Antibodies

### Binding of Trispecific CD79bxCD20xCD3 Antibodies to Pan T-Cells

Binding of the CD3 arm of CD79bxCD20xCD3 trispecific constructs was assessed using cryo-preserved, negatively selected, primary human CD3⁺ pan T cells. Primary human CD3⁺ pan T cells from three different donors were incubated for 1 hour with CD79bxCD20xCD3 test molecules C923B169 and C923B168 (1 uM starting concentration at 1:3 serial dilutions) at 37° C. All cells were washed with BD stain buffer (BD Biosciences; Cat #554657), centrifuged at 1200 RPM for 3 mins, with supernatant discarded. Cells were then stained for 20 minutes at 4° C. with BD stain buffer containing AlexaFluor 647 labeled anti-human IgG secondary antibody (Jackson Immuno; Cat #109-606-098) at a 1:300 dilution. All cells were washed with BD stain buffer (BD Biosciences; Cat #554657), centrifuged at 1200 RPM for 3 mins, with supernatant discarded. Cells were resuspended in 50 ul of FACS buffer containing a 1:1000 dilution of Cytox Green viability dye (Invitrogen, Cat #S34860). Cells were analyzed using Intellicyt (Sartorius) flow cytometer and mean fluorescent intensity (MFI) was generated using Forcyt software (Sartorius). MFI was graphed using GraphPad PRISM v.8. Dose response curves were generated by transforming the x axis values using the formula x=lox. Data was then graphed using non-linear regression curve fit analysis “log(agonist) vs. response-variable slope (four parameter)”.

All CD79bxCD20xCD3 molecules showed binding on all donor Pan T cells expressing endogenous CD3 on the cell surface, shown in Table 25.

TABLE 25
C923B169 and C923B168 CD79b × CD20 × CD3
binding to Pan CD3 T cells.

| | Pan T cell binding, | | | Pan T cell max binding, | | |
|---|---|---|---|---|---|---|
| Construct | EC50 (nM) | | | MFI (×10⁶) | | |
| Name | Donor 1 | Donor 2 | Donor 3 | Donor 1 | Donor 2 | Donor 3 |
| C923B168 | 125 | 91 | 97 | 1.3 | 1.0 | 1.0 |
| C923B169 | UD* | UD* | UD* | 0.1 | 0.03 | 0.04 |

UD* = undetermined

FACS T Cell Killing Data on Panel of Target Positive (CD79b⁺ and CD20⁺) Cell Lines

Functional activity of the CD79bxCD20xCD3 trispecific constructs was assessed at 48 and 72 hr time point in an in vitro T cell killing assay by flow cytometry using cell lines that were validated by flow cytometry to have different endogenous expression levels of CD79b and CD20 on the cell surface, shown in Table 26.

TABLE 26
CD79b and CD20 Antigen Density of B Lymphoma Cell Lines

| | | | CD20 Antigen |
|---|---|---|---|
| | | CD79b Antigen | Density |
| | | Density (Antigen | (Antigen |
| Cell Line | Cell Type | Number/cell) | Number/cell) |
| OCI-LY10 | Diffuse large B-cell | 38,885 | 67,352 |
| | lymphoma line | | |
| CARNAVAL | Diffuse large B-cell | 98,176 | 118,789 |
| | lymphoma line | | |
| JEKO-1 | Mantle cell lymphoma | 280,000 | 50,000 |

Target cancer cells were maintained in complete RPMI-1640 (ThermoFisher, catalog #11875093) media containing 10% heat inactivated fetal bovine serum. Prior to the assay, antibodies were made at 3-fold serial dilutions in RPMI 1640 media containing 10% heat inactivated fetal bovine serum, at 4-fold expected final concentration. A volume of 50 μL of medium-diluted bispecific or trispecific Ab in each well of a 96-well plate were further diluted into 200 μL by adding a mix of target and effector cell suspension. The target cell lines were harvested by centrifuge at 400×g for 5 min, washed one time with RPMI 1640 media. Target cancer cells were stain targets with CellTrace CFSE (ThermoFisher; Cat #: C34554) diluted 1/5000. Healthy donor T cells (isolated by CD3-negative selection provided by Discovery Life Sciences) were thawed in complete media (RPMI 1640 media containing 10% heat inactivated fetal bovine serum), counted and suspended in fresh complete phenol red-free RPMI 1640 media at 1×10⁶ cells/mL. Target cells and T cells were mixed to obtain 5:1 effector to target cell ratio. Cell suspension was added to antibody dilution wells according to plate layout (150 μL/well). Cells were incubated for 48 and 72 hours with CD79bxCD20xCD3 test molecules C923B169 and C923B168 (100 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 15 minutes at room temperature with Fixable Live/Dead stain (ThermoFisher; Cat #65-0865-14) at a 1:1000 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 27), antibodies amount added as listed in the table. All cells were washed with BD stain buffer (BD Biosciences; Cat #554657), centrifuged at 1200 RPM for 3 mins, with supernatant discarded. Cells were analyzed using FACS Lyric (BD) flow cytometer and percent of cancer cell killing was generated using Cytobank. Percent of cancer cell killing was graphed and IC50 values generated using GraphPad PRISM v.8. Dose response curves were generated by transforming the x axis values using the formula x=lox. Data was then graphed using non-linear regression curve fit analysis “log(inhibitor) vs. response-variable slope (four parameter)”.

TABLE 27
Flow Panel Antibodies for T cell killing Assay

| | | | | | Amount |
|---|---|---|---|---|---|
| Antibody | Conjugated | | Catalog | LOT | added per |
| Name | Fluorophore | Vendor | Number | Number: | well (μl) |
| CD4 | V500 | BD | 560768 | 9340575 | 2 μl/well |
| | | Biosciences | | | |
| CD8 | PerCPCy5.5 | BD | 560662 | 9290508 | 2 μl/well |
| | | Biosciences | | | |
| CD69 | PE | BD | 560968 | 9049603 | 10 μl/well |
| | | Biosciences | | | |
| CD25 | BV421 | BD | 562443 | 10302 | 2 μl/well |
| | | Biosciences | | | |

CD79b × CD20 × CD3 trispecific mediated potent cytotoxicity. IC50 (nM) values and Max killing values are listed in Table 28 and Table 29.

TABLE 28
C923B169 and C923B168 CD79b × CD20 × CD3 killing of target positive
(CARNAVAL, OCI-Ly10, JEK0-1) cell lines (FACS) at 48 hours.
IC50 (nM) and percent of maximal killing are listed in the table.
Average values from 2 independent T cell donors.

| | Killing | | Killing | | Killing | | Killing | | Killing OCI- | | Killing OCI- | |
|---|---|---|---|---|---|---|---|---|---|---|---|---|
| | CARNAVAL | | CARNAVAL | | JEKO-1 1:1 | | JEKO-1 5:1 | | Ly10 1:1 | | Ly10 5:1 | |
| | 1:1 48 hr | | 5:1 48 hr | | 48 hr | | 48 hr | | 48 hr | | 48 hr | |
| Protein ID | IC50 | Max | IC50 | Max | IC50 | Max | IC50 | Max | IC50 | Max | IC50 | Max |
| C923B169 | 110.3 | 49.7% | 0.179 | 87.2% | 27.307 | 60.8% | 0.027 | 93.5% | >100 | 22.2% | >100 | 19.7% |
| C923B168 | 13.6 | 58.8% | 0.012 | 95.0% | 7.466 | 65.3% | 0.002 | 96.7% | >100 | 25.1% | >100 | 29.8% |

TABLE 29
C923B169 and C923B168 CD79b × CD20 × CD3 killing of target positive
(CARNAVAL, OCI-Ly10, JEK0-1) cell lines (FACS) at 72 hours.
IC50 (nM) and percent of maximal killing are listed in the table.
Average values from 2 independent T cell donors.

| | Killing | | Killing | | Killing | | Killing | | Killing OCI- | | Killing | |
|---|---|---|---|---|---|---|---|---|---|---|---|---|
| | CARNAVAL; | | CARNAVAL; | | JEKO-1; | | JEKO-1; | | Ly10; | | OCI-Ly10; | |
| | 1:1 72 hr | | 5:1 72 hr | | 1:1 72 hr | | 5:1 72 hr | | 1:1 72 hr | | 5:1 72 hr | |
| Protein ID | IC50 | Max | IC50 | Max | IC50 | Max | IC50 | Max | IC50 | Max | IC50 | Max |
| C923B169 | 50.15 | 66.8% | 0.026 | 98.3% | 0.087 | 82.2 | 0.003 | 99.4% | >100 | 43.5% | 24.58 | 70.0% |
| C923B168 | 15.53 | 75.9% | 0.003 | 99.3% | 0.010 | 85.8% | 0.001 | 99.6% | 0.81 | 61.0% | 0.51 | 90.7% |

### C923B169 and C923B168 CD79bxCD20xCD3 Mediated Cytotoxicity Against Autologous B-Cells

Functional activity of the C923B169 and C923B168 CD79bxCD20xCD3 constructs was assessed in an in vitro autologous B cell depletion assay. This functional assay utilizes PBMCs to focus on the killing of primary B cells as well as T cell activation on donor matched primary cells. Cryo-preserved PBMCs from 3 different human donors were incubated for 72 hours with CD79bxCD20xCD3 test molecules C923B169 and C923B168 (300 nM starting concentration at 1:3 serial dilutions) at 37° C. All cells were washed with BD stain buffer (BD Biosciences; Cat #554657), centrifuged at 1200 RPM for 3 minutes, with supernatant discarded. Cells were stained for 10 minutes at room temperature with BD stain buffer containing Fc blocking agent (Accurate Chemical and Scientific Corp; Cat #NB309) and Near IR Fixable Live/Dead stain (Invitrogen; Cat #L10119) at a 1:400 dilution. All cells were washed with BD stain buffer (BD Biosciences; Cat #554657), centrifuged at 1200 RPM for 3 mins, with supernatant discarded. Cells were then stained for 30 minutes at 4° C. with BD stain buffer containing flow panel antibodies (Table 30) at a 1:100 dilution. All cells were washed with BD stain buffer (BD Biosciences; Cat #554657), centrifuged at 1200 RPM for 3 mins, with supernatant discarded. Cells were analyzed using Intellicyt (Sartorius) flow cytometer. EC50 values generated using GraphPad PRISM v.8. Dose response curves were generated by transforming the x axis values using the formula x=lox. Data was then graphed using non-linear regression curve fit analysis “log(agonist) vs. response-variable slope (four parameter)”.

TABLE 30
Flow Panel Antibodies for Autologous B Cell Depletion Assay

| | Conjugated | | Catalog |
|---|---|---|---|
| Antibody Name | Fluorophore | Vendor | Number |
| Anti-human CD25 | BV650 | BD Biosciences | 563719 |
| Anti-Human CD4 | BV510 | Biolegend | 317444 |
| Anti-Human CD8 | PE-Cy7 | Biolegend | 301012 |
| Anti-Human CD20 | PE | Biolegend | 302306 |
| Anti-Human CD11c | AF647 | BD Biosciences | 565911 |
| Anti-Human CD2 | BV605 | BD Biosciences | 740391 |

CD79bxCD20xCD3 C923B169 and C923B168 constructs showed a maximum drug mediated cytotoxicity of 69-95 percent (Table 31) with low levels of CD4⁺ and CD8⁺ T cell activation as demonstrated by CD25 expression on these T cell subsets.

TABLE 31
C923B169 and C923B168 CD79b × CD20 × CD3 killing of B cell
in the primary autologous B cell depletion assay at 72 hours.
EC50 (nM) and percent of maximal killing are listed in the
table. Values from 3 independent T cell donors listed.

| | | | | | | | Average | |
|---|---|---|---|---|---|---|---|---|
| | D329465 | | D198013 | | D221837 | | values | |
| | | Max | | Max | | Max | | Max |
| | EC50 | Kill | EC50 | Kill | EC50 | Kill | EC50 | Kill |
| Name | (nM) | (%) | (nM) | (%) | (nM) | (%) | (nM) | (%) |
| C923B168 | 0.1 | 69 | 0.02 | 92 | 0.1 | 95 | 0.07 | 84 |
| C923B169 | 2.0 | 69 | 1.70 | 92 | 6.30 | 81 | 2.80 | 80 |

### Example 8: C923B169

The trispecific antibody C923B169 (CD9B374 FabxC20B648 LH scFv x CD3B2030 N106A LH scFv) is a fully human IgG1 trispecific mAb that simultaneously binds to the epsilon subunit of the CD3 TCR complex (i.e., CD3ε; Uniprot ID P07766), and to CD79b (i.e., B-cell antigen receptor complex-associated protein R chain; Uniprot ID P40259) and CD20 (i.e., B-lymphocyte antigen CD20; Uniprot ID P11836) on tumor cells. The antibody features mutations of L234A, L235A, and D265S (AAS) in the constant region (i.e., fragment crystallizable [Fc]) to abolish interaction with Fc receptors. Heterodimerization is enhanced using the knobs-into-holes platform mutations. The molecule comprises an anti-CD3ε single-chain variable fragment (scFv) fused onto the N-terminus of the ‘knob’ Fc region (i.e., T366W) and an anti-CD20 scFv attached to the C-terminus of the Fc. The ‘hole’ chain (i.e., T366S, L368A, Y407V) features an anti-CD79b fragment antigen-binding (Fab) and contains ‘RF’ mutations (i.e., H435R, Y436F) to disrupt Protein A binding of monomeric and homodimerized hole chains (mutations numbered according to EU numbering).

Table 32 provides recombinant antigen binding, species cross-reactivity, CD79b and CD3 epitope identification, Fc receptor binding, and biophysical intrinsic property attributes for C923B169. The trispecific molecule C923B224 (SEQ ID NO: 263) was generated. C923B224 is identical to C923B169 with an added Histidine at the C-terminus of HC1 (SEQ ID NO: 260) The biophysical characteristics for C923B224 were essentially similar to C923B169.

### Biophysical Assessment of C923B169

TABLE 32
List of biophysical assessments and results for C923B169.

| | | |
|---|---|---|
| mAb TMP characteristic | Result | Comments |

Binding

| | | |
|---|---|---|
| Binding affinity (SPR) | Human CD79b: K_{D} = 0.16 nM; CD3: | CD20 binding: the Acro- |
| | no quantitative results by SPR due to | biotinylated CD20 antigen had |
| | biphasic profile; Antigens: | little to no measurable activity |
| | CD9W7.001 and CD3W220.001 | due to technical challenge. |
| | | Affinity binding was assessed |
| | | using cell-based method on |
| | | CARNAVAL cells instead, with |
| | | EC₅₀ determined to be 2.1 nM for |
| | | Fab and 16 nM for scFv. Binding |
| | | may be repeated on target KO |
| | | cells upon further cell line |
| | | validation. |
| | | CD3 binding: weak binding to |
| | | CD3 protein was detected, but K_{D} |
| | | could not be determined due to |
| | | lack of fit of the binding curve. |
| | | Affinity binding was further |
| | | evaluated on pan T cells (see |
| | | details below) |
| Cross-reactivity of the | Cyno CD79b: weak binding, K_{D} | Binding data were collected from |
| CD9B374 binder | estimated >3 μM; Mouse CD79b: no | C923B38 containing the same |
| | binding (up to 3 μM antigen | CD79b Fab arm |
| | concentration) | |
| FcRn binding (SPR) | pH 6.0: hu FcRn K_{D} = 890 nM; cyno | Rapid dissociation at pH 7.4 |
| | FcRn K_{D} = 818 nM | |
| Serum interference (Octet) | No significant change in association | The Acro-biotinylated CD20 |
| | rate to bt-CD79b and bt-CD3 in 50% | antigen had little to no |
| | human serum; kₐ buffer/kₐ serum | measurable activity due to |
| | <2.0 | technical challenge. |
| CD79b binding epitope | Epitope encompassing residues of | The residue numbers correspond |
| | P40259 at segments 30-42 | to P40259 (CD79B_Human); the |
| | (SEDRYRNPKGSAC; SEQ ID NO: | CD79b construct used for the |
| | 253), 50-52 (PRF), 81-86 (EMENP; | epitope mapping was CD9W7 |
| | SEQ ID NO: 254), and 144-148 | |
| | (GFSTL; SEQ ID NO: 255) | |
| CD3 binding epitope | Epitope encompassing residues of | The residue numbers correspond |
| | P07766 at segments 54-58 (GSEIL; | to P07766 (CD3E_Human); |
| | SEQ ID NO: 257), 74-75 (NI), and | similar to OKT3's epitope; the |
| | 100-105 (PRGSKP; SEQ ID NO: 258) | CD3 construct used for the |
| | | epitope mapping was CD3W220 |

Protein characterization

| | | |
|---|---|---|
| Intact trispecific Ab mass, | 156,308.2 Da | N/A |
| release (MS) | | |
| Glycoform profile (MS) | Typical IgG1 profile with main | N/A |
| | glycoform GOF/GOF | |
| Level of homodimer(s) (MS) | None detected | N/A |
| Levels of other product-related | None detected | N/A |
| impurities (MS) | | |
| N-link gly (non-Fc) (MS) | None predicted nor detected | N/A |
| O-link gly sites (MS) | None detected | N/A |
| Glycation (% relative | CD79b HC: 13.8%; CD79b LC: 6.0%; | N/A |
| abundance) (MS) | CD3 HC: 32.2% | |
| Free Cys (MS) | None predicted nor detected | N/A |
| N-terminal | None detected | N/A |
| elongation/truncation (MS) | | |
| Conformational stability (DSC) | Tₘ₁ = 68.4° C.; Tₘ₂ = 75.1° C.; Tₘ₃ = | Measured in 10 mM L-histidine |
| | 77.7° C. | pH 6.5 |
| % Purity (2-step purification) | >94% Monomer (monomer presents 2 | Measured in 10 mM L-histidine |
| (AUC) | peaks in AUC, most likely due to | pH 6.5 |
| | conformational isoforms) | |
| Serum stability (SEC-FDS) | ~0.5% increase in HMW and LMW | Assessed by SEC using |
| | after 7-day incubation at 37° C. | Alexa488-labeled C923B169 |
| IgG interactions (SPR) | No IgG interactions detected | N/A |
| Non-specific binding | No non-specific binding detected | N/A |
| Isoelectric point (cIEF) | pI = 8.44; % acidic/main/basic peak | N/A |
| | area: 35.5/58.5/6.0 | |
| Relative hydrophobicity (aHIC) | Relatively low/medium | N/A |
| | hydrophobicity (HI = 0.67) | |
| Viscosity | 6.1 cP | Determined at 100 mg/mL in |
| | | 10 mM L-histidine pH 6.5 with |
| | | 0.04% PS20 |

2-week high concentration stability (4 and 40° C., target 150 mg/mL in 10 mM L-histidine buffer with
0.04% PS20 and 50 mg/mL in PBS)

| | | |
|---|---|---|
| Concentratability and % | Sample was concentrated to | Determined in 10 mM L-histidine |
| recovery in L-histidine | 162 mg/mL with 84% recovery | pH 6.5 with 0.04% PS20 |
| Concentratability and % | Sample was concentrated to | Determined in PBS |
| recovery in PBS | 65 mg/mL with 90.5% recovery | |
| % Monomer (initial) (AUC) | 96.4% | Determined in 10 mM L-histidine |
| | | pH 6.5 with 0.04% PS20 |
| % Monomer 2 weeks, 4° C. | 95.5% | Determined in 10 mM L-histidine |
| (AUC) | | pH 6.5 with 0.04% PS20 |
| % Monomer 2 weeks, 40° C. | 63.7% | Determined in 10 mM L-histidine |
| (AUC) | | pH 6.5 with 0.04% PS20 |

Chemical (PTM) and physical stability

| | | |
|---|---|---|
| Intact MS after forced | Trace losses from N- and C-terminus | Observed fragmentation is |
| degradation (MS) | of both HCs seen under high pH | insignificant due to very low |
| | stress, while a trace loss of the | levels detected. |
| | C-terminal domain of HCl scFv was | |
| | also observed under physiological | |
| | stress. | |
| Size after forced degradation | Samples maintained >97.0% monomer | N/A |
| (aSEC) | under stressing conditions, except for: | |
| | Thermal: 30.3% aggregate at | |
| | 162 mg/mL in L-histidine and 14.7% | |
| | aggregates at 65 mg/mL in PBS after | |
| | 2 weeks at 40° C.; High pH: 1.5% | |
| | aggregates and ~16.6% fragment after | |
| | 1 week at pH 8.5 37° C. | |
| Purity forced degradation (R | All stressed samples maintained >95% | N/A |
| and NR GXII) | intact, expect high pH sample | |
| | (~27.5% LMW) | |
| Basal oxidation level | 2.4% M95 (CD9B374 LC CDR3); | N/A |
| | 3.0% in M98 (CD9B374 LC CDR3); | |
| | <1.2% for all other CDR residues | |
| Met/Trp oxidation under stress | 3.3% in M95 (CD9B374 LC CDR3); | N/A |
| (H₂O₂) | 89.8% in M94 (CD9B374 LC CDR3) | |
| | along with a 3.3-fold weaker binding | |
| | affinity but no changes in % activity | |
| Basal deamidation level | <0.1% in CDRs | N/A |
| Deamidation under stress | No significant change | N/A |
| (pH 8.5) | | |
| Basal isomerization level | <1% in CDRs | N/A |
| Isomerization under stress | No significant change | N/A |
| (pH 5) | | |

Ab, antibody;
aHIC, analytical hydrophobic interaction chromatography;
aSEC, analytical size-exclusion chromatography;
AUC, analytical ultracentrifugation;
bt, biotinylated;
CD, cluster of differentiation;
CDR, complementarity determining region;
cIEF, capillary isoelectric focusing;
cyno, cynomolgus monkey;
Cys, cysteine,
DSC, differential scanning calorimetry;
EC₅₀, 50% effective concentration;
Fab, fragment antigen-binding;
Fc, fragment crystallizable;
FcRn, neonatal Fc receptor;
FDS, fluorescence detection system;
gly, glycosylation;
HC, heavy chain;
HMW, higher molecular weight;
hu, human;
Ig, immunoglobulin;
ka, association constant;
KD, equilibrium dissociation constant;
KO, knock out;
LC, light chain;
LMW, lower molecular weight;
mAb, monoclonal antibody;
Met, methionine;
MS, mass spectrometry;
N/A, not assessed;
NR GXII, non-reduced capillary electrophoresis;
PBS, phosphate-buffered saline;
pI, isoelectric point;
PS20, Polysorbate 20;
ref, reference;
R GXII, reduced capillary electrophoresis;
scFv, single-chain variable fragment;
SEC, size-exclusion chromatography;
SPR, surface plasmon resonance;
Tm, melting temperature;
TMP, target medicinal product;
Trp, tryptophan.

### Target Arm and CD3 Arm Binding Characterization of C923B169

### Endogenous Tumor Cell Line Binding of C923B169

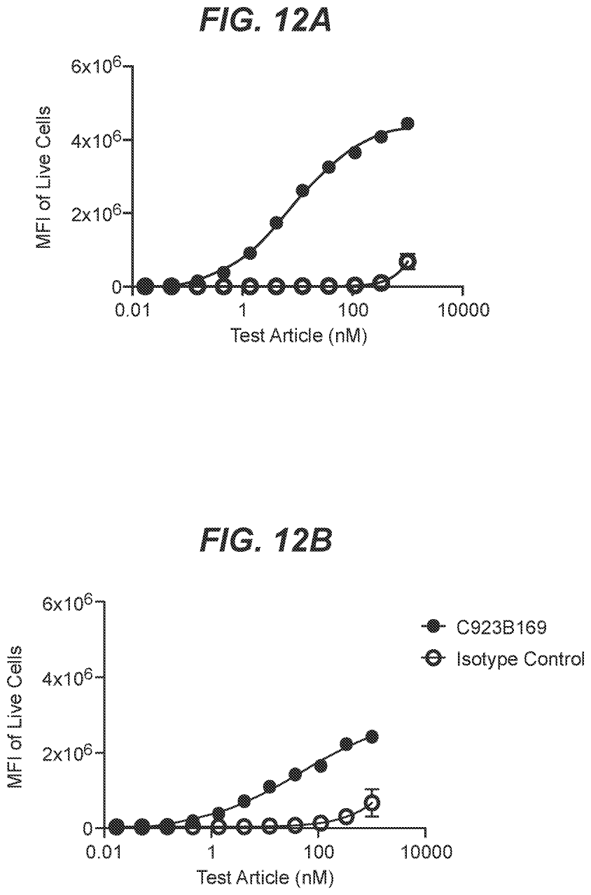

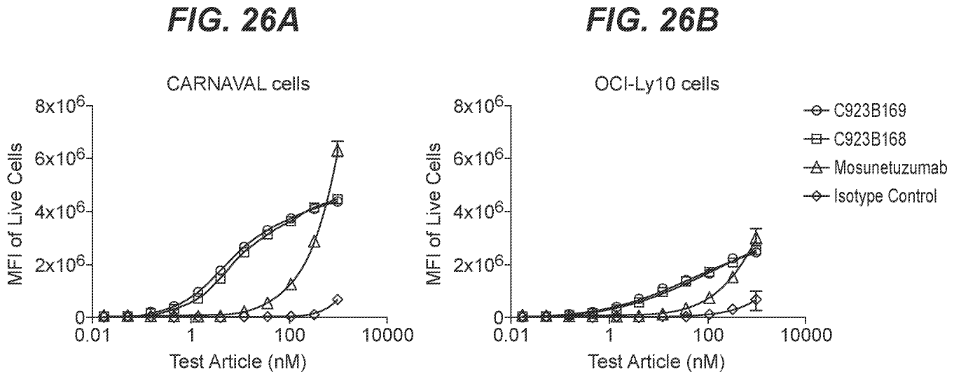

Flow cytometry was used to evaluate the CD79b and CD20 arm binding of C923B169 in vitro. C923B169 and isotype were tested on a panel of endogenous cell lines of varying CD79b and CD20 receptor densities after 1-hour 37° C. incubation. C923B169 showed good binding on cell lines expressing endogenous CD79b and CD20 on the cell surface at a range of antigen densities (FIGS. 12A-12B, Table 33). C923B169 also illustrated better binding potency compared to mosunetuzumab (see FIGS. 26A-26B, Table 34).

TABLE 33
Calculated receptor counts and ECₓ values for
endogenous tumor cell line binding.

| | CD79b | CD20 | Type | Cell binding EC₂₀, EC₅₀, | | |
|---|---|---|---|---|---|---|
| | receptor | receptor | of cell | and EC₉₀ values (nM) | | |
| Cell lines | count | count | line | EC₂₀ | EC₅₀ | EC₉₀ |
| CARNAVAL | 165,541 | 429,801 | DH DLBCL | 1 | 8 | 216 |
| Jeko-1 | 617,117 | 334,371 | MCL | 1.6 | 20.3 | 1,098 |
| WILL-2 | 4,304 | 756 | DH DLBCL | I | I | I |
| OCI-Ly10 | 63,633 | 424,335 | ABC | 2.3 | 43.4 | 4,791 |
| | | | DLBCL | | | |
| SU-DHL1 | −438 | −919 | ALCL | NA | NA | NA |
| K562 | −546 | −1,309 | CML | NA | NA | NA |

ABC, activated B-cell;
ALCL, anaplastic large-cell lymphoma;
B-NHL, B-cell non-Hodgkin lymphoma;
CD, cluster of differentiation;
CML, chronic myelogenous leukemia;
DH, double-hit;
DLBCL, diffuse large B-cell lymphoma;
ECₓ, x % effective concentration;
I, indeterminate;
MCL, mantle cell lymphoma;
NA, not assessed;
PE, phycoerythrin.
CD79b and CD20 receptor density results in CD79b and CD20 target positive B-NHL cell lines were evaluated. CD79b expression was determined by flow cytometry using a PE-labeled commercial antibody (human CD79B-PE clone # SN8; BD Bioscience #335833), CD20 expression was determined by flow cytometry using a PE-labeled commercial antibody (human CD79B-PE clone # 2H7; BD Bioscience #555623), and receptor counts were calculated using the Quantum Simply cellular kit (Bangslabs #815). EC₂₀, EC₅₀, and EC₉₀ values for endogenous tumor cell binding were assessed.

TABLE 34
Calculated cell binding EC₂₀, EC₅₀, and EC₉₀ values (nM)
for CARNAVAL and OCI-Ly10 binding.

| | C923B169 | | | C923B168 | | | Mosunetuzumab | | |
|---|---|---|---|---|---|---|---|---|---|
| | EC₂₀ | EC₅₀ | EC₉₀ | EC₂₀ | EC₅₀ | EC₉₀ | EC₂₀ | EC₅₀ | EC₉₀ |
| CARNAVAL | 1.0 | 8.0 | 216.0 | 1.0 | 11.0 | 267.0 | I | I | I |
| OCI-Ly10 | 2.3 | 43.4 | 4,791.0 | 4.0 | 55.0 | 5,000.0 | I | I | I |

ECₓ, x % effective concentration;
I, indeterminate.

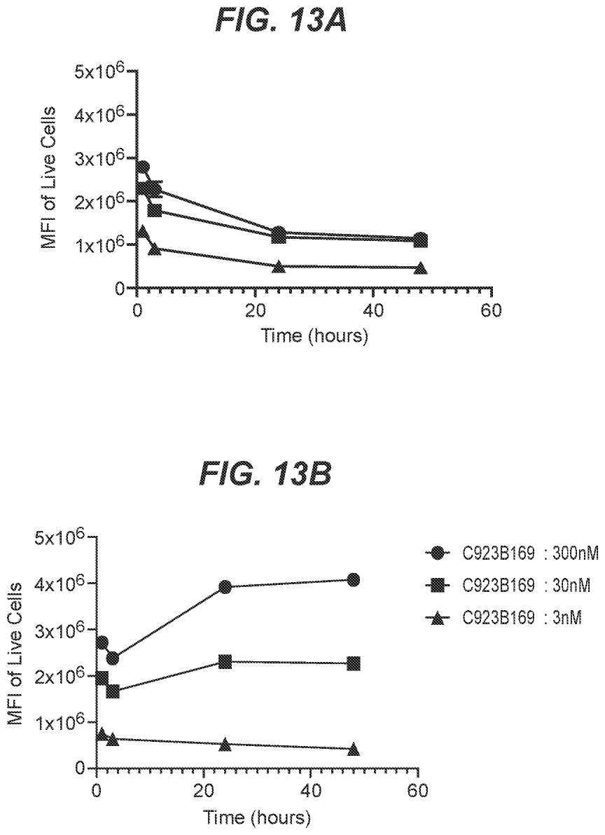

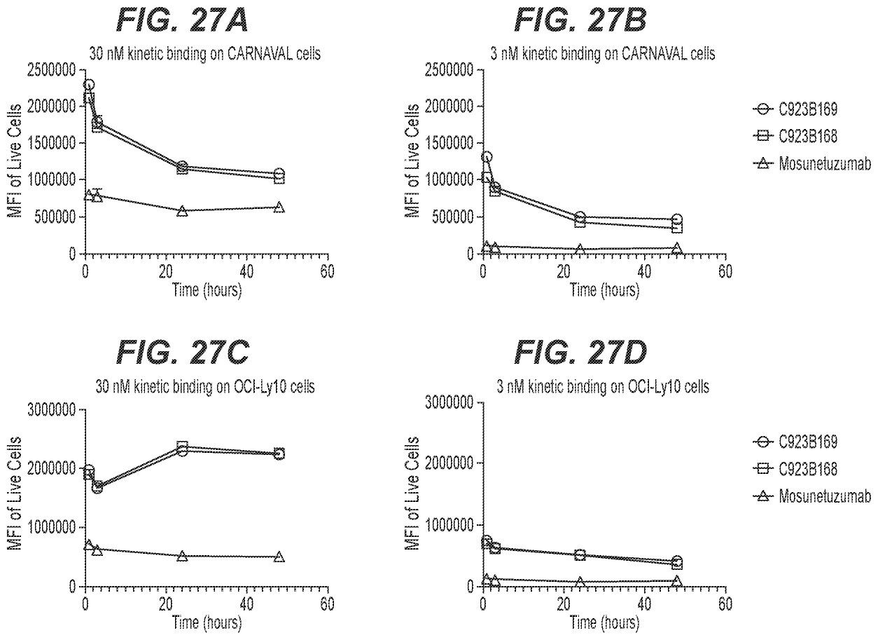

Flow cytometry was used to evaluate the CD79b and CD20 arm binding kinetic of C923B169 in vitro. C923B169 and isotype were tested in parallel on CARNAVAL, OCI-Ly10, and WILL-2 (data not shown) cell lines at 300, 30, and 3 nM throughout 48-hour 37° C. incubation. C923B169 showed stable binding profiles at all tested concentrations to tumor cell lines co-expressing endogenous CD79b and CD20 on the cell surface (FIGS. 13A-13B). C923B169 also illustrated superior tumor arm binding stability compared to mosunetuzumab (see FIGS. 27A-27D).

### Primary T-Cell Binding of C923B169

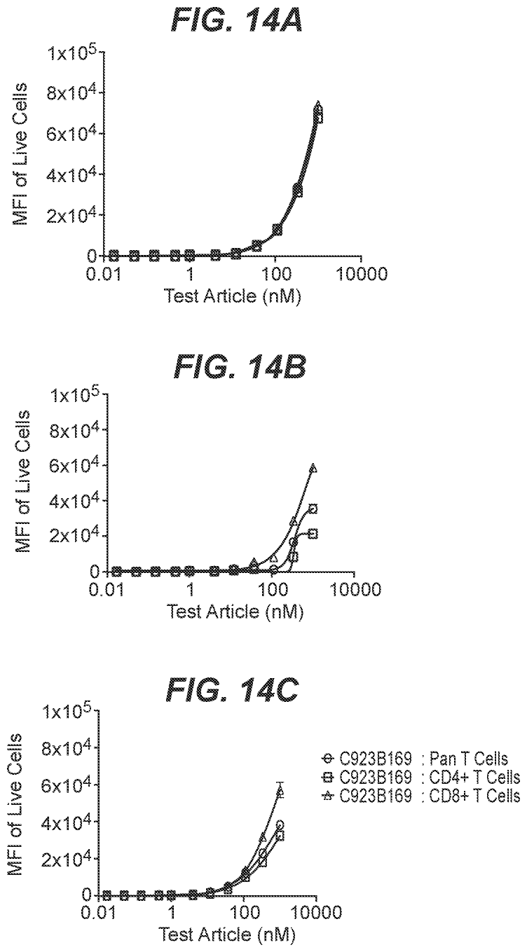

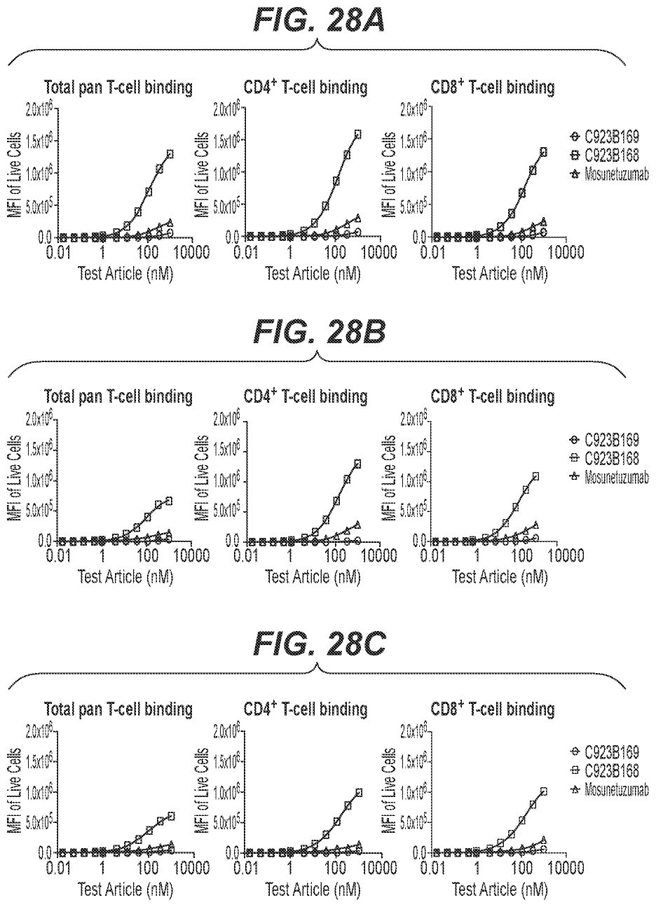

Flow cytometry was used to evaluate the CD3 arm binding of C923B169 to CD3⁺ pan T cells from 3 different healthy human donors. C923B169 and isotype were tested after 1-hour 37° C. incubation in pan T cells. C923B169 showed consistent binding profiles across primary T-cell populations expressing endogenous CD3 on the cell surface (FIGS. 14A-14C, Table 35). C923B169 showed slightly weaker binding with all tested donor T cells compared to mosunetuzumab (see FIGS. 28A-28C, Table 36).

TABLE 35
Max MFI values for primary T-cell binding of C923B169.

| | Mean of Max MFI ± SD (×10⁵) |
|---|---|
| Pan T-cell binding | 0.5 ± 0.2 |
| CD4⁺ T-cell binding | 0.4 ± 0.2 |
| CD8⁺ T-cell binding | 0.6 ± 0.1 |

Max, maximum;
MFI, mean fluorescence intensity;
SD, standard deviation.

TABLE 36
Max MFI values for primary T-cell binding.

| | Mean Max MFI ± SD (×10⁶) | | |
|---|---|---|---|
| | C923B169 | C923B168 | Mosunetuzumab |
| Pan T-cell binding | 0.05 ± 0.02 | 0.87 ± 0.39 | 0.13 ± 0.06 |
| CD4⁺ T-cell binding | 0.04 ± 0.02 | 1.30 ± 0.30 | 0.23 ± 0.11 |
| CD8⁺ T-cell binding | 0.06 ± 0.01 | 1.13 ± 0.15 | 0.23 ± 0.05 |

Max, maximum;
MFI, mean fluorescence intensity;
SD, standard deviation.
C923B168 has an EC₅₀ value of 104 ± 18 nM.

In conclusion, C923B169 is a fully human trispecific mAb targeting the TCR CD3 with 1 binding arm and tumor cell surface antigens CD79b or CD20 with the remaining 2 binding domains. C923B169 showed acceptable intrinsic biophysical properties and bound to all tested CD79b- and CD20-expressing cell lines. Binding profiles and reported EC values were dependent on cell surface antigen density. C923B169 showed stable tumor cell binding profiles over 48 hours. C923B169 also showed measurable, low-affinity binding to primary human T cells expressing CD3 on the cell surface.

### Example 9: Therapeutic Pharmacology

### C923B169-Induced T-Cell-Mediated Cytotoxicity of B-Cell Lymphoma Lines In Vitro

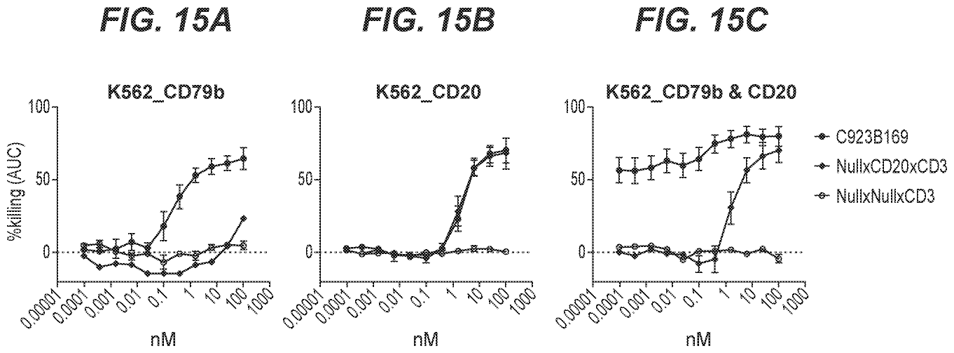

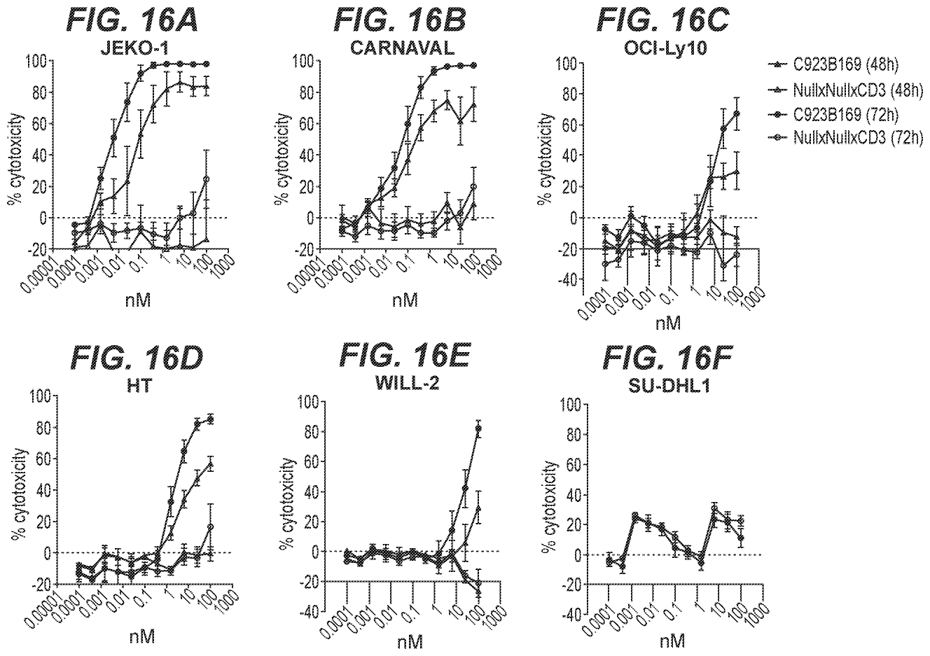

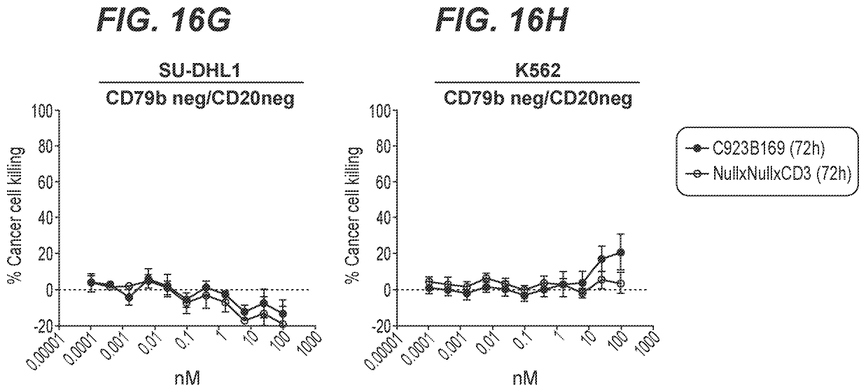

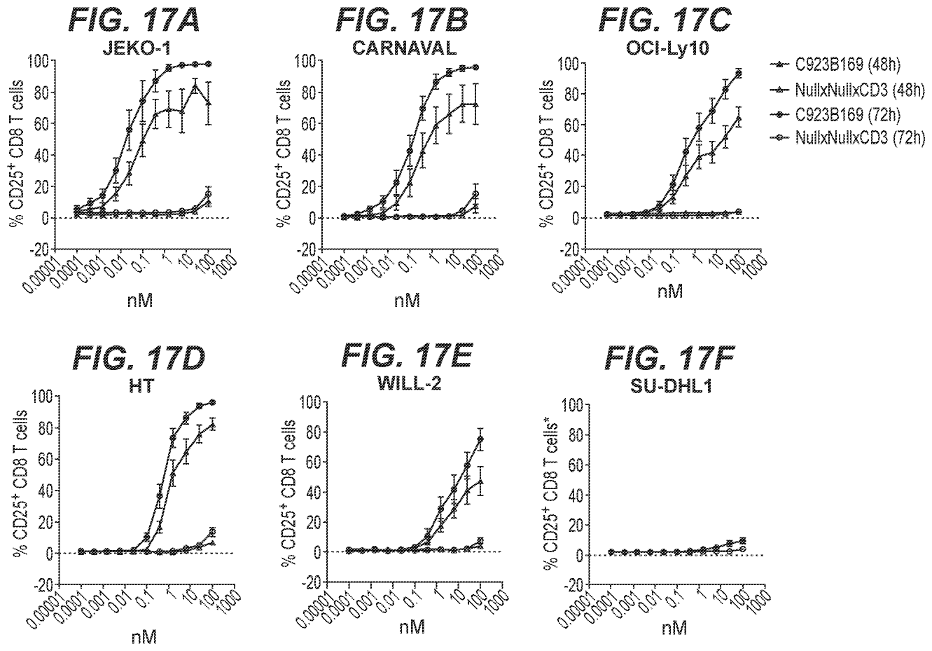

To validate that CD79bxCD20xCD3 trispecific antibodies could target cancer cells expressing CD79b or CD20 only, as well as to show that increased efficacy is observed when both antigens are present, K562 models either overexpressing CD79b (FIG. 15A) or CD20 (FIG. 15B) alone, or both targets together, were generated (FIG. 15C). Using long-term assays, T-cell-mediated cytotoxicity was assessed in presence of C923B169 or matched NullxCD20xCD3 control. In the K562 CD79b overexpression model, cytotoxicity was only observed with C923B169 while the matched NullxCD20xCD3 antibody was inactive (FIG. 15A, Table 37). In the K562 CD20 overexpression model, C923B169 and the matched NullxCD20xCD3 control showed overlapping activity (FIG. 15B, Table 37). When both CD79b and CD20 antigens were present in the K562 CD79b/CD20 overexpression model, a clear benefit of using C923B169 was observed as compared to the CD20-targeting antibody resulting in ˜100-fold 50% effective concentration (EC₅₀) shifts, confirming an avidity effect (FIG. 15C, Table 37). High expression levels of CD79b and CD20 in the K562 isogenic models may contribute to the strong avidity effect observed. No cytotoxicity was observed when using the K562 parental cells (see FIG. 16H) in a 72-hour fluorescence activated cell sorting (FACS) cytotoxicity assay.

TABLE 37
EC50 values (nM) for long-term Incucyte cytotoxicity of CD79b/CD20
isogenic K562 cell lines.

| | K562 CD79b | K562 CD20 | K562 CD79b&CD20 |
|---|---|---|---|
| C923B169 | 1.04 | 4.08 | <0.0001 |
| Null × CD20 × CD3 | >100 | 3.58 | 3.19 |
| Null × Null × CD3 | >100 | >100 | >100 |

EC₅₀, 50% effective concentration.

To assess the cytotoxicity potential of C923B169 in B-NHL models in vitro, a FACS-based approach was utilized to measure tumor cell viability in the presence of test compound and purified human pan T cells. Selected B-cell lymphoma cell lines, expressing varying levels of CD79b and CD20 representing DLBCL and MCL, were used to assess efficacy. SU-DHL-1 (anaplastic large cell lymphoma) and K562 (chronic myelogenous leukemia [CML]) cells were used as target-negative cell lines (Table 33 lists the receptor density values across a panel of cell lines). HT (GCB DLBCL) cells are reported to express low levels of CD79b and medium levels of CD20. Confirmation of HT receptor density measurement is currently ongoing. Naïve CD3⁺ pan T cells from healthy donors were combined with individual cell lines at a 5:1 effector-to-target (E:T) ratio and treated with varying concentrations of C923B169 for either 48 or 72 hours. C923B169 was able to elicit cytotoxicity of CD79b⁺/CD20⁺ cell lines after 48 and 72 hours, but not of the CD79b⁻/CD20⁻ cell lines (FIGS. 16A-16H). A NullxNullxCD3 negative control antibody had no to minimal effect on cytotoxicity; minimal effects were observed in some cellular models at the top concentration tested (i.e., 100 nM). The up-and-down behavior of the curve observed for target-negative cell line SU-DHL1 across all antibodies tested, including NullxNullxCD3 control antibodies, was clearly attributed to a FACS technical acquisition issue.

To assess the impact of increased target burden on the cytotoxicity potential of C923B169 in vitro, a FACS-based approach was utilized to measure tumor cell viability in the presence of treatment and purified human pan CD3⁺ T cells at a 1:1 E:T ratio for either 48 or 72 hours. Both maximum cytotoxicity and EC₅₀ values of C923B169 were decreased and increased, respectively, at a 1:1 ratio in comparison to a 5:1 ratio (Table 38).

TABLE 38
C923B169-induced T-cell-directed cytotoxicity: EC₅₀ and maximum
cytotoxicity values with different cancer cell lines at different
E:T ratios at 48 and 72 hours.

| 48 hours: mean values | | | | | 72 hours: mean values | | | | |
|---|---|---|---|---|---|---|---|---|---|
| JEKO- | CAR- | OCI- | | WILL- | JEKO- | CAR- | OCI- | | WILL- |
| 1 | NAVAL | Ly10 | HT | 2 | 1 | NAVAL | Ly10 | HT | 2 |

E:T ratio 5:1

| | | | | | | | | | | |
|---|---|---|---|---|---|---|---|---|---|---|
| EC₅₀ | 0.149 | 0.237 | 2.221 | 4.186 | NE | 0.0092 | 0.092 | 27.042 | 2.016 | 31.971 |
| (nM) | | | | | | | | | | |
| Max kill | 90.8 | 78.4 | 31.0 | 59.1 | NE | 99.0 | 98.5 | 81.8 | 86.8 | 95.5 |
| (%) | | | | | | | | | | |

E:T ratio 1:1

| | | | | | | | | | | |
|---|---|---|---|---|---|---|---|---|---|---|
| EC50 | 0.588 | 0.658 | 94.462* | NA | NA | 0.155 | 0.536 | 16.543 | NA | NA |
| (nM) | | | | | | | | | | |
| Max kill | 65.8 | 35.0 | 62.8* | NA | NA | 85.9 | 77.8 | 49.3 | NA | NA |
| (%) | | | | | | | | | | |

EC₅₀, 50% effective concentration;
E:T ratio, effector-to-target ratio;
Max kill, maximum cytotoxicity;
NA, not assessed;
NE, not estimable with the NLME statistical model;
NLME, non-linear mixed effects;
*high variability across tested donors.
Values are representative of 5 to 6 individual healthy donors. Data from independent experiments were pooled and represented as mean. A NLME model was used for EC₅₀ value estimation across the donors. Separate NLME models were fit for each time point, treatment, and titration. The NLME model was used to estimate dose-response profile, using the 4 parameters of the standard 4-parameter logistic regression model as the fixed effects (minimum, maximum, slope, logEC₅₀) and using donor as a random effect. EC₅₀ values were derived from this model, along with donor-specific EC₅₀ using post-hoc estimation. Results are reported on the original scale. All analyses were performed in R Version 4.0.3.

### C923B169-Mediated T-Cell Activation In Vitro

To assess the level of T-cell activation in the cytotoxicity assays described above, CD25 expression was measured on T cells in the same assays. T cells activation was induced in the presence of C923B169 only when incubated with CD79b⁺/CD20⁺ cell lines, but not in the presence of the CD79b⁻/CD20⁻ cell lines, demonstrating the specificity of T-cell activation (FIGS. 17A-17B). SU-DHL1 is a T-cell lymphoma with reported constitutive expression of interleukin (IL)-2 receptor (CD25) and secretion of IL-2 (36). This SU-DHL1 intrinsic feature corresponds to higher basal CD25 levels in T cells in coculture with this particular cell line independent of CD3 redirection antibody presence. C923B169 did not contribute to further increase in the level of T-cell activation in SU-DHL1 cells. Similarly, a negative control NullxNullxCD3 antibody did not induce significant T-cell activation in any of the cell lines.

The impact of lower E:T ratio on T-cell activation was also assessed, by measuring CD25 expression on T cells. EC₅₀ values for T-cell activation were similar for most of the cell lines at 1:1 and 5:1 E:T ratios in the presence of C923B169 (Table 39).

TABLE 39
C923B169-induced CD8 T-cell activation: EC₅₀ values from T-cell
cytotoxicity assays with different cancer cell lines at different
E:T ratios at 48 and 72 hours.

| | 48 hours: mean values | | | | | 72 hours: mean values | | | | |
|---|---|---|---|---|---|---|---|---|---|---|
| E:T | JEKO- | CAR- | OCI- | | WILL- | JEKO- | CAR- | OCI- | | WILL- |
| ratio | 1 | NAVAL | Ly10 | HT | 2 | 1 | NAVAL | Ly10 | HT | 2 |
| 5:1 | 0.097 | 0.465 | 3.925 | 1.648 | 5.048 | 0.068 | 0.185 | 2.386 | 0.638 | 12.651 |
| 1:1 | 0.099 | 0.207 | 0.295 | NA | NA | 0.098 | 0.107 | 0.386 | NA | NA |

EC₅₀, 50% effective concentration;
E:T ratio, effector-to-target ratio;
NA, not assessed.
Values are representative of 5 to 6 individual healthy donors. A non-linear mixed effect model was used to estimate dose-response using the 4 parameters of standard 4-parameter logistic regression model as fixed (minimum, maximum, slope, and logEC₅₀) and using donor as a random effect. EC₅₀ (nM) values were derived using post-hoc estimation. Data from independent experiments were pooled and represented as mean.

### Impact of Longer Incubation on C923B169 Cytotoxicity of CARNAVAL and OCI-Ly10 Cells

As the limited time frame of in vitro assays might not reflect the potency of CD3 redirectors specifically with low CD3 affinity, long-term cytotoxic effects of C923B169 were assessed. To this end, CD3⁺ T cells were cocultured with red fluorescent protein (RFP)-expressing CARNAVAL or OCI-Ly10 target cells at 2 different E:T ratios (i.e., 5:1 and 1:1). Cancer cell proliferation/viability was assessed for 2, 3, and 6 days taking advantage of the Incucyte technology by measuring over time the RFP⁺ area in each well. The area under the curve (AUC) was calculated to evaluate cancer cell growth over the indicated time. EC₂₀, EC₃₀, and EC₅₀ values were calculated over specific time frames (Table 40). In almost all conditions analyzed, the EC₂₀, EC₃₀, and EC₅₀ values decreased over prolonged time, indicative of increased long-term potency.

TABLE 40
C923B169-induced T-cell-directed kinetic cytotoxicity
(Incucyte) values of CARNAVAL and OCI-Ly10
cells at different E:T ratios after 2, 3, and 6 days.

| | | OCI-Ly10 | | | CARNAVAL | | |
|---|---|---|---|---|---|---|---|
| | | 2 days | 3 days | 6 days | 2 days | 3 days | 6 days |
| | | Model | Model | Model | Model | Model | Model |
| | | est. | est. | est. | est. | est. | est. |
| E:T | | (nM): | (nM): | (nM): | (nM): | (nM): | (nM): |
| ratio | ECₓ | mean | mean | mean | mean | mean | mean |
| 5:1 | EC₂₀ | 3.74 | 1.01 | 0.33 | 1.79 | 1.22 | 1.02 |
| | EC₃₀ | 32.59 | 2.65 | 0.61 | 3.10 | 1.45 | 1.06 |
| | EC₅₀ | >100 | >100 | 1.98 | >100 | 2.90 | 1.16 |
| 1:1 | EC₂₀ | >100 | 3.42 | 2.07 | 0.60 | 0.22 | 0.10 |
| | EC₃₀ | >100 | 16.37 | 3.50 | 1.36 | 0.49 | 0.18 |
| | EC₅₀ | >100 | >100 | 17.14 | >100 | 5.02 | 0.44 |

ECₓ, x % effective concentration;
est., estimated;
E:T ratio, effector-to-target ratio;
RFP, red fluorescent protein.
Cytotoxicity to cancer cells was assessed over time using Incucyte technology after coculture of T cells with RFP-expressing CD79b⁺/CD20⁺ cancer cells at the indicated E:T ratios. Average ECₓ values from 2 independent donor over specific time are listed in the table.

### C923B169-Induced Cytokine Release in the Presence of CARNAVAL and OCI-Ly10 Cell Lines at 5:1 and 1:1 Effector-to-Target Ratio

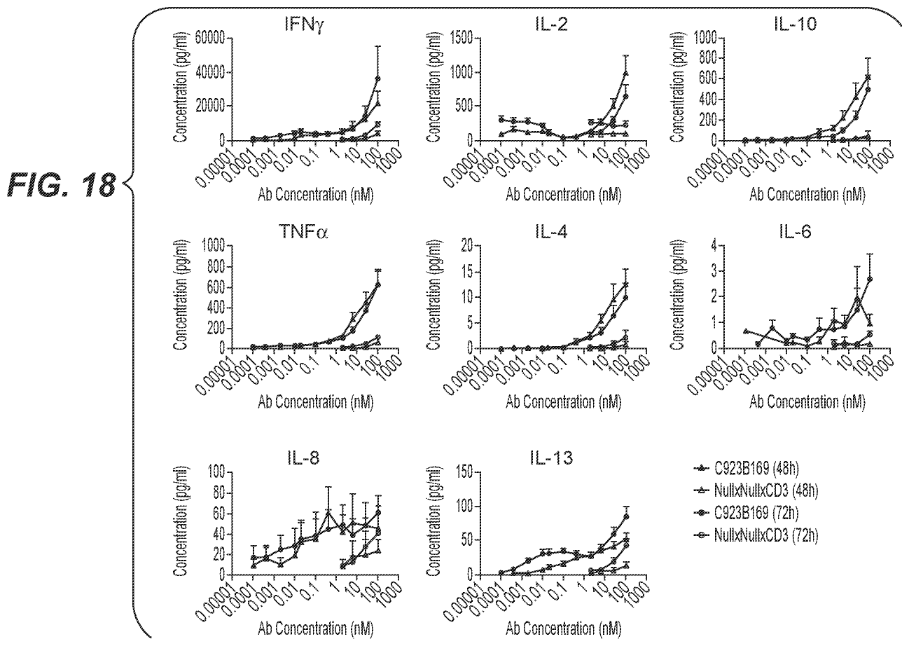

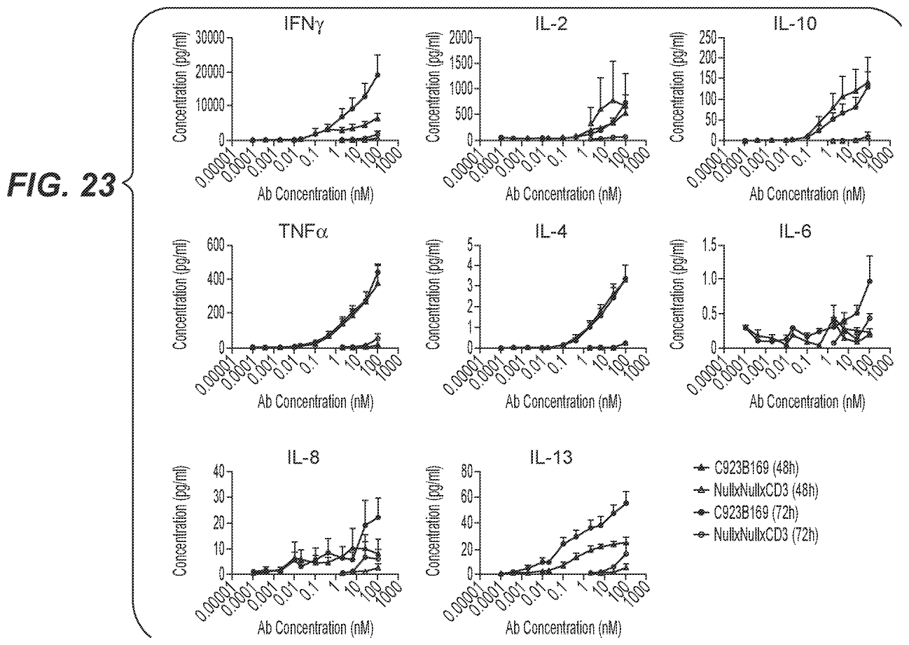

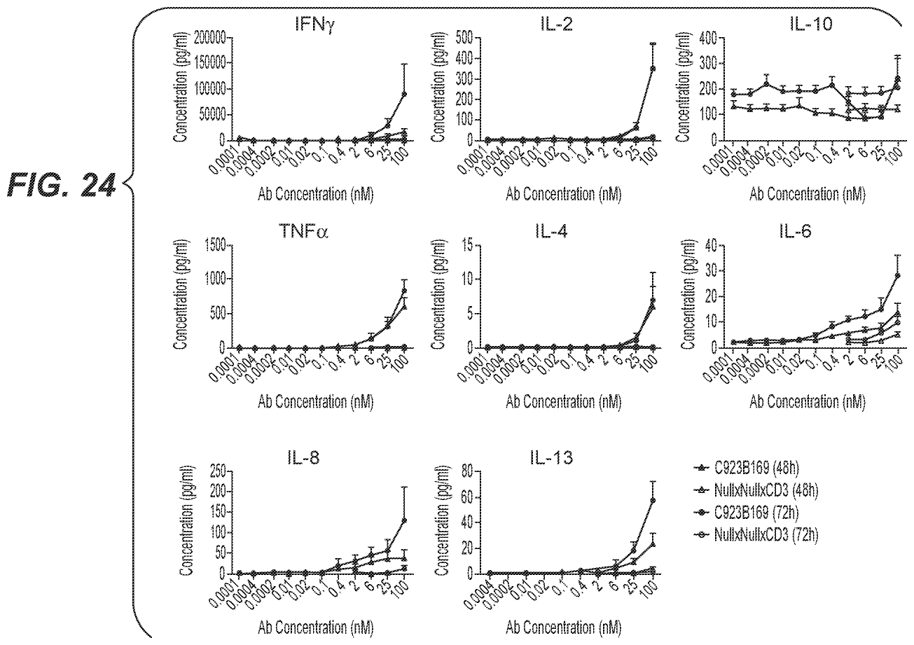

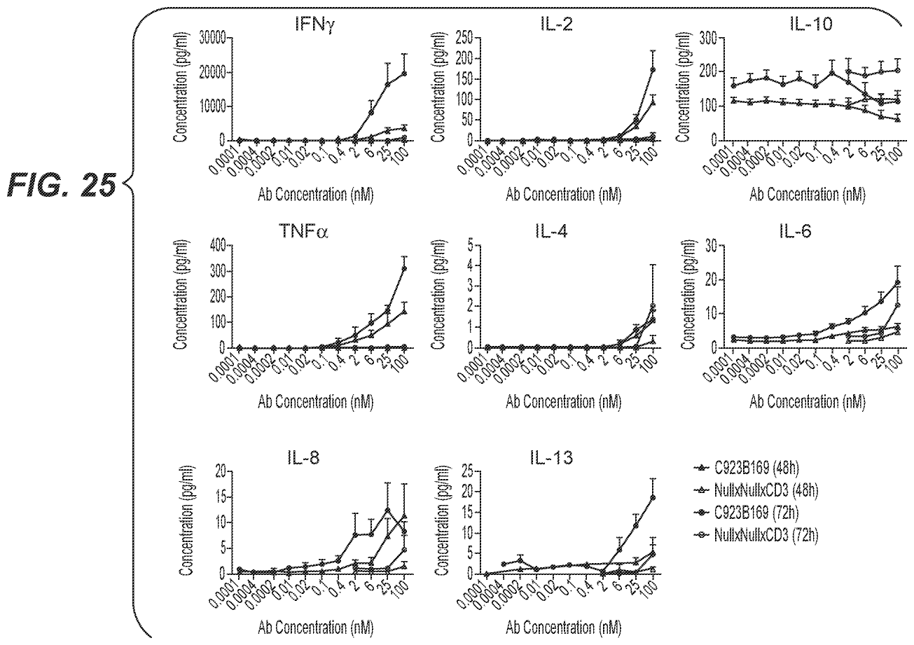

To further characterize T-cell activation induced by C923B169, supernatants from the in vitro cytotoxicity assay were analyzed for cytokine levels using a Meso Scale Discovery (MSD) enzyme-linked immunosorbent assay (ELISA) Proinflammatory Panel 1. Overview of the data for T-cell-mediated cytokine release to CARNAVAL cells at an E:T ratio of 5:1 are shown in FIG. 18 and the EC₅₀ and corresponding maximal cytokine values are shown in Table 41. Similar results were obtained for assessment of cytokines at lower 1:1 E:T ratio and during OCI-Ly10 T-cell-mediated cytotoxicity at both 1:1 and 5:1 E:T ratio (see FIGS. 23-25, Tables 42-43). As hypothesized, weak engagement of CD3 resulted in T-cell activation with lower cytokine release across tested cell lines in in vitro T-cell cytotoxicity assays as compared to the trispecific C923B168, which has medium affinity for CD3 (pan T-cell binding EC₅₀=104±18 nM), and mosunetuzumab (see FIGS. 28A-28C, Table 44).

TABLE 41
C923B169-induced inflammatory cytokines in the presence of CARNAVAL
and OCI-Ly10 cells at different E:T ratios at 48 and 72 hours (EC₅₀ [nM] and
maximal cytokine release [pg/mL] values)).

| | | 48-hour incubation | | | | 72-hour incubation | | | |
|---|---|---|---|---|---|---|---|---|---|
| | | CARNAVAL | | OCI-Ly10 | | CARNAVAL | | OCI-Ly10 | |
| Cyto- | | E:T ratio | | | | | | | |
| kine | Value | 1:1 | 5:1 | 1:1 | 5:1 | 1:1 | 5:1 | 1:1 | 5:1 |
| IFN-γ | EC₅₀ | 2.097 | NE | 36.339 | 55.646 | 1.571 | 48.338 | 13.744 | 85.887 |
| | Max value | 4,213.3 | NE | 5,630.8 | 16,564.2 | 12,185.5 | 30,242.4 | 16,383.0 | 59,934.1 |
| IL-1β | EC₅₀ | NE | NE | ND | ND | NE | NE | ND | ND |
| | Max value | NE | NE | ND | ND | NE | NE | ND | ND |
| IL-2 | EC₅₀ | 2.871 | 15.516 | 62.195 | >100* | >100* | >100* | >100* | >100* |
| | Max value | 386.8 | 747.7 | 105.0 | 461.7 | 1,005.3 | 1,468.5 | 0.1 | 1,253.9 |
| IL-4 | EC₅₀ | 1.894 | 7.946 | NE | NE | 14.044 | 37.367 | NE | NE |
| | Max value | 2.1 | 11.1 | NE | NE | 3.0 | 11.1 | NE | NE |
| IL-8 | EC₅₀ | NE | NE | 15.050 | 1.944 | NE | NE | >100* | 1.878 |
| | Max value | NE | NE | 5.3 | 19.1 | NE | NE | 23.2 | 32.8 |
| IL-6 | EC₅₀ | NE | NE | 1.251 | NE | NE | NE | 8.545 | NE |
| | Max value | NE | NE | 5.5 | NE | NE | NE | 17.1 | NE |
| IL-10 | EC₅₀ | 1.047 | >100* | NE | NE | 0.939 | >100* | NE | NE |
| | Max value | 81.0 | 1,173.8 | NE | NE | 67.2 | 223,016.3 | NE | NE |
| IL-13 | EC₅₀ | 0.429 | 3.392 | NE | NE | 0.202 | 17.497 | NE | NE |
| | Max value | 0.2 | 42.5 | NE | NE | 42.9 | 77.3 | NE | NE |
| TNF-α | EC₅₀ | 2.158 | 60.226 | >100* | >100* | 10.573 | >100* | 33.971 | >100* |
| | Max value | 282.0 | 980.4 | 234.6 | 1,310.3 | 397.0 | 2,382.7 | 344.8 | 3,143.2 |

CFSE, carboxyfluorescein succinimidyl ester;
EC₅₀, 50% effective concentration;
E:T ratio, effector-to-target ratio;
IFN, interferon;
IL, interleukin;
MSD, Meso Scale Discovery;
Max value, maximal cytokine release value;
ND; not detectable in 5 out of 6 donors;
NE, not estimable;
TNF, tumor necrosis factor.
CFSE-labeled CARNAVAL cells were added to CD3 T cells at 5:1 or 1:1 E:T ratio for either 48 or 72 hours with increasing concentrations of C923B169. Supernatants were analyzed for inflammatory cytokines using a custom MSD Proinflammatory kit (MSD K15049D). Values are representative of 6 individual healthy donors. EC₅₀ estimates come from a non-linear mixed effects model and standard 4-parameter logistic regression model as the fixed effects (minimum, maximum, slope, and logEC₅₀) and using donor as a random effect. EC₅₀ values could not be estimated for cytokines for which non-sigmoidal or decreasing response was observed and were listed in the table as NE. The model was fit using log-transformed data, estimates of interest were back-transformed to the original scale for reporting.
*EC₅₀ values estimated to be higher than highest tested concentration.

TABLE 42
C923B169-induced lower levels of inflammatory cytokines as compared to
C923B168 and mosunetuzumab (MSCB782.001) in the presence of CARNAVAL and
OCI-Ly10 cells at different E:T ratios at 48 and 72 hours (EC₅₀ values [nM]).

| | | 48 hour incubation | | | | 72 hour incubation | | | |
|---|---|---|---|---|---|---|---|---|---|
| | | CARNAVAL | | OCI-Ly10 | | CARNAVAL | | OCI-Ly10 | |
| Cyto- | | E:T | | | | | | | |
| kine | Antibody | 1:1 | 5:1 | 1:1 | 5:1 | 1:1 | 5:1 | 1:1 | 5:1 |
| IFN-γ | C923B169 | 2.097 | NE | 36.339 | 55.646 | 1.571 | 48.338 | 13.744 | 85.887 |
| | C923B168 | 0.451 | 0.774 | 0.522 | 3.642 | 25.541 | 4.864 | 1.244 | 3.263 |
| | Mosunetuzumab | 0.201 | 14.483 | 0.174 | 0.639 | 0.625 | >100* | 0.367 | 0.931 |
| IL-2 | C923B169 | 2.871 | 15.516 | 62.195 | >100* | >100* | >100* | >100* | >100* |
| | C923B168 | 1.520 | 1.160 | >100* | 77.461 | 2.123 | 2.588 | 28.409 | 26.548 |
| | Mosunetuzumab | 0.794 | 1.094 | 1.464 | 6.430 | 4.947 | 2.035 | 3.457 | 9.436 |
| TNF-α | C923B169 | 2.158 | 60.226 | >100* | >100* | 10.573 | >100* | 33.971 | >100* |
| | C923B168 | 0.292 | 1.774 | 7.505 | 10.138 | 1.359 | 2.090 | 5.349 | 11.121 |
| | Mosunetuzumab | 0.362 | 1.398 | 0.549 | 1.944 | 2.840 | 2.790 | 0.629 | 1.711 |

EC₅₀, 50% effective concentration;
ET ratio, effector-to-target ratio;
IFN, interferon;
IL, interleukin;
NE, not estimable;
TNF, tumor necrosis factor.
Values are representative of 5 to 6 individual healthy donors. A non-linear mixed effect model was used to estimate dose-response using the 4 parameters of standard 4-parameter logistic regression model as fixed (minimum, maximum, slope, and logEC₅₀) and using donor as a random effect. EC₅₀ (nM) values were derived using post-hoc estimation. Data from independent experiments were pooled and represented as mean.
*EC₅₀ values estimated to be higher than highest tested concentration.

TABLE 43
Comparison of C923B169-induced CD8 T-cell activation as compared to
C923B168 and mosunetuzumab (MSCB782.001): EC₅₀ values of CD8 T-cell
activation from T-cell cytotoxicity assays with different cancer cell lines at different
E:T ratios at 48 and 72 hours.

| 48 hours: mean values | | | | | 72 hours: mean values | | | | |
|---|---|---|---|---|---|---|---|---|---|
| JEKO- | CAR- | OCI- | | WILL- | JEKO- | CAR- | OCI- | | WILL- |
| 1 | NAVAL | Ly10 | HT | 2 | 1 | NAVAL | Ly10 | HT | 2 |

E:T ratio 5:1

| | | | | | | | | | | |
|---|---|---|---|---|---|---|---|---|---|---|
| C923B169 | 0.097 | 0.465 | 3.925 | 1.648 | 5.048 | 0.068 | 0.185 | 2.386 | 0.638 | 12.651 |
| C923B168 | 0.051 | 1.901 | 2.346 | 0.859 | 0.885 | 0.020 | 0.079 | 0.815 | 0.451 | 2.288 |
| Mosunetuzumab | 0.043 | 0.038 | 0.256 | 0.148 | 1.237 | 0.019 | 0.021 | 0.120 | 0.078 | 1.268 |

E:T ratio 1:1

| | | | | | | | | | | |
|---|---|---|---|---|---|---|---|---|---|---|
| C923B169 | 0.099 | 0.207 | 0.295 | NA | NA | 0.098 | 0.107 | 0.386 | NA | NA |
| C923B168 | 0.021 | 0.063 | 0.141 | NA | NA | 0.031 | 0.032 | 0.087 | NA | NA |
| Mosunetuzumab | 0.022 | 0.053 | 0.045 | NA | NA | 0.021 | 0.013 | 0.035 | NA | NA |

EC₅₀, 50% effective concentration;
E:T ratio, effector-to-target ratio;
NA, not assessed.
Values are representative of 5 to 6 individual healthy donors. A non-linear mixed effect model was used to estimate dose-response using the 4 parameters of standard 4-parameter logistic regression model as fixed (minimum, maximum, slope, and logEC₅₀) and using donor as a random effect. EC₅₀ (nM) values were derived using post-hoc estimation. Data from independent experiments were pooled and represented as mean.

TABLE 44
Max MFI values for primary T-cell binding.

| | Mean Max MFI ± SD (×10⁶) | | |
|---|---|---|---|
| | C923B169 | C923B168 | Mosunetuzumab |
| Pan T-cell binding | 0.05 ± 0.02 | 0.87 ± 0.39 | 0.13 ± 0.06 |
| CD4⁺ T-cell binding | 0.04 ± 0.02 | 1.30 ± 0.30 | 0.23 ± 0.11 |
| CD8⁺ T-cell binding | 0.06 ± 0.01 | 1.13 ± 0.15 | 0.23 ± 0.05 |

Max, maximum;
MFI, mean fluorescence intensity;
SD, standard deviation.
C923B168 has an EC₅₀ value of 104 ± 18 nM.

### C923B169-Induced T-Cell-Mediated Cytotoxicity of Primary B Cells in the Autologous Setting (B-cell Depletion Assay)

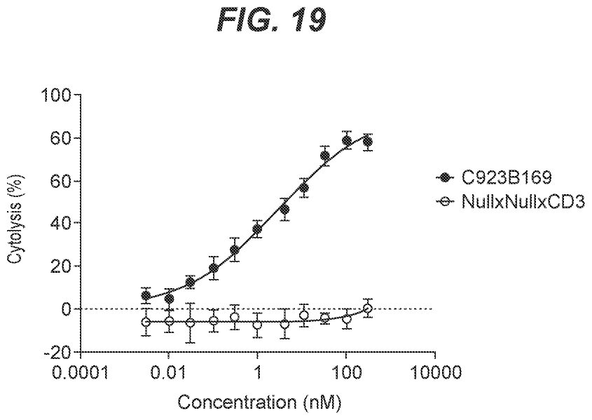

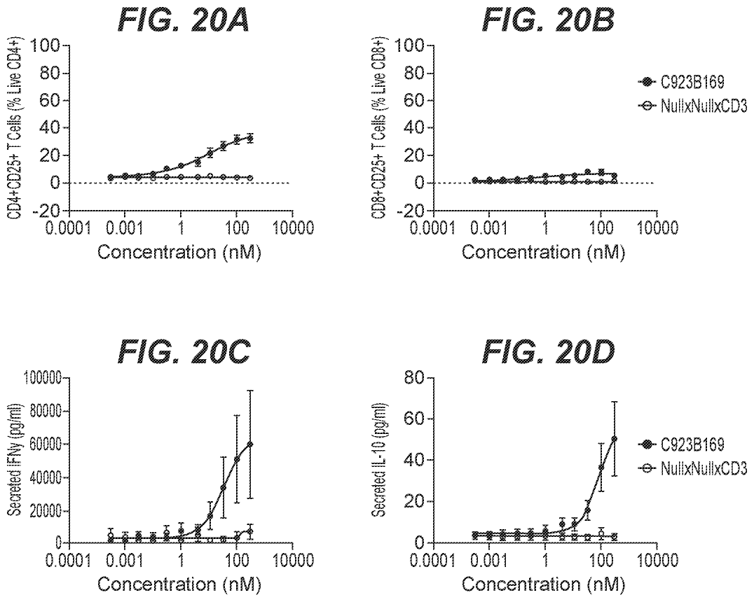

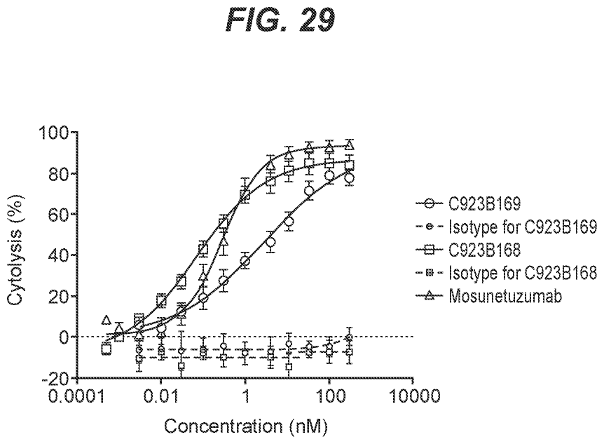

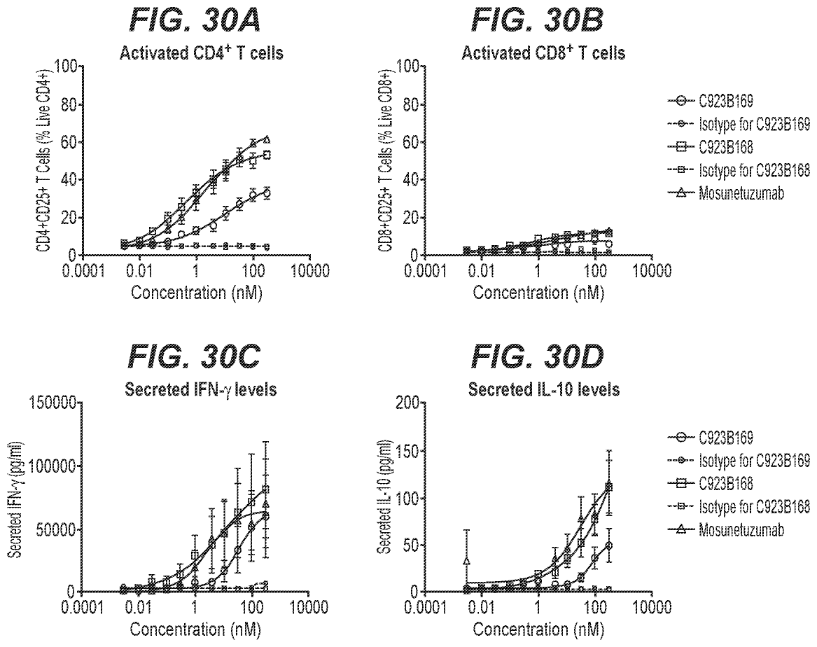

The cytotoxicity of C923B169 against autologous B cells was evaluated with peripheral blood mononuclear cells (PBMCs) from 3 different normal human donors. C923B169 was incubated with PBMCs from each donor for 72 hours at 37° C., followed by the assessment of cytotoxic and T-cell activation effectiveness. The E:T ratio of the individual donors was 1:1 for donor D327645, 1:1 for donor D198013, and 1:2 for donor D221837. C923B169 showed robust cytotoxicity of autologous B cells (FIG. 19), with an EC₅₀ value of 3.3±2.6 nM and a maximum cytotoxicity value of 81%±11%. T-cell activation with lower levels of secreted cytokines than for mosunetuzumab were measured, showing the desired T-cell profiles for C923B169 (FIGS. 20A-20D; see FIG. 29, FIGS. 30A-30D, Table 45).

TABLE 45
Autologous B-cell depletion assay calculated
EC₅₀ values and maximum cytotoxicity.

| | D329465 | | D198013 | | D221837 | |
|---|---|---|---|---|---|---|
| | EC₅₀ | Max | EC₅₀ | Max | EC₅₀ | Max |
| | (nM) | kill (%) | (nM) | kill (%) | (nM) | kill (%) |
| C923B169 | 2.0 | 69 | 1.70 | 92 | 6.30 | 81 |
| C923B168 | 0.1 | 69 | 0.02 | 88 | 0.1 | 95 |
| Mosunetuzumab | 0.2 | 86 | 0.2 | 97 | 0.5 | 98 |

ECₓ, x % effective concentration; Max kill, maximum cytotoxicity.

### Antagonistic Activity of C923B169

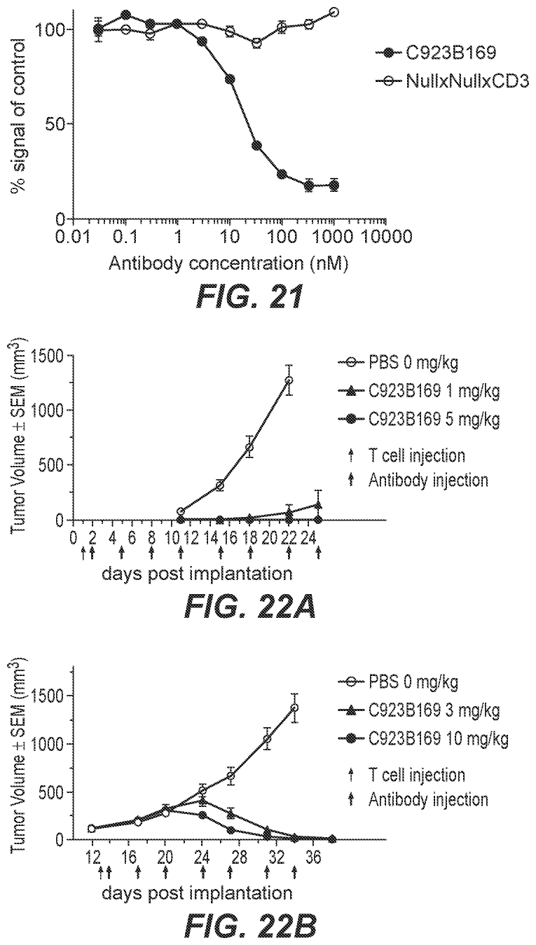

Phosphorylation of CD79a and CD79b initiates BCR signaling. One of the prominent downstream signaling pathways engaged after BCR stimulation is the classical NF-κB pathway, which is frequently activated in ABC DLBCL due to oncogenic mutations in CD79a/b or caspase recruitment domain family member 11 (CARD11). NF-κB signaling regulates the expression of multiple cytokines, including IL-10. Secretion of IL-10 by CD79b mutant OCI-Ly10 (ABC DLBCL) cells was measured using a MSD assay. Inhibition of NF-κB signaling by C923B169 resulted in a decrease of IL-10 secretion indicative of antagonistic activity of C923B169 (FIG. 21). The EC₅₀ value of C923B169 was 15.43 nM.

C923B169-induced T-cell-mediated Cytotoxicity of B-cell Lymphoma Xenografts in Vivo

The antitumor efficacy of C923B169 was evaluated in SC human double-hit (DH) DLBCL CARNAVAL (Study A) and human ABC DLBCL OCI-Ly10 xenografts (Study B) in T-cell-humanized mice. For all studies, female NSG (i.e., non-obese diabetic [NOD] severe combined immunodeficiency [scid] or NOD.Cg Prkdc^{scid} Il2rg^{tm1Wjl}/SzJ) mice (Charles River Labs, Lyon, France) were used to provide a suitable host for reconstituting a human immune system using human donor CD3⁺ pan T cells. Mice were inoculated with 1×10⁶ CARNAVAL or OCI-Ly10 lymphoma tumor cells SC prior to T-cell engraftment. The implant day was designated as Day 0 of the study.

Expanded human pan T cells were inoculated intraperitoneally (IP) into NSG mice to humanize the immune system. Humanization of mice with CD3-expressing T cells provided effector cells to facilitate C923B169-mediated cytotoxicity to human CD79b/CD20-expressing tumor cells. T-cell-humanized mice were given Fc block IP and intravenous immunoglobulin (IVIg) IP at least 30 minutes prior to C923B169 dosing to correct for the low Ig environment in NSG mice.

Efficacy Study A (FIG. 22A) served as a tumor growth prevention study. NSG mice were randomly assigned into groups of 10 animals, implanted SC with CARNAVAL cells, humanized with pan T cells on Day 1 post cell implantation and treated with C923B169 IP from Day 2 post cell implantation onwards at 1 and 5 mg/kg twice weekly for a total of 8 doses. Percent tumor growth inhibition (TGI) of SC CARNAVAL xenografts was calculated on Day 22 post tumor implantation, when >66% of control animals remained on study. Statistically significant TGI was observed with C923B169 at 1 and 5 mg/kg resulting in 94.6% (p<0.0001) and 100% (p<0.0001) TGI, respectively, as compared with vehicle-treated controls (phosphate-buffered saline [PBS]).

In efficacy Study B (FIG. 22B), NSG mice were randomized into groups of 10 animals and humanized with T cells 13 days post OCI-Ly10 SC cell implantation with starting tumor volumes averaging 108 mm³. IP treatment with 3 and 10 mg/kg of C923B169 per animal was initiated on Day 14 post OCI-Ly10 cell implantation twice weekly for a total of 7 doses. Percent ΔTGI of SC OCI-Ly10 xenografts was calculated on Day 34 post tumor implantation, when >66% of control animals remained on study. Statistically significant ΔTGI was observed with C923B169 at 3 and 10 mg/kg resulting in 94.6% (p<0.0001) and 100% (p<0.0001) ΔTGI, respectively, as compared with vehicle-treated controls (PBS). Tumor regression as compared to initial tumor burden of SC OCI-Ly10 xenografts was evaluated on Day 34 and 38 (i.e., 3 days post final dose). Tumor regression was observed with C923B169 at 3 and 10 mg/kg resulting in 69.1% (p<0.001) and 86.6% (p<0.001) tumor regression, respectively, at Day 34, progressing to almost complete tumor regression of 91.2% (p<0.001) and 98.1% (p<0.001) respectively, at Day 38 compared to initial tumor burden.

In both studies, treatment with C923B169 at 1 or 5 mg/kg (for Study A up to Day 25) and 3 or 10 mg/kg (for Study B up to Day 38) did not show signs of body weight loss or other signs of gross toxicity. First signs of graft-versus-host disease (GvHD)-related morbidity due to the humanization with T cells was observed on Day 38 in Study B, but not in Study A.

Taken together, the in vitro and in vivo results presented above document C923B169's ability to induce potent and antigen-specific cytotoxicity to cancer cells and tumor regression.

In vitro, C923B169 led to cytotoxicity to cancer cells in a broad panel of NHL cell models showing different levels of CD79b and CD20 surface expression, both in long- and short-term assays. No impact on cancer cell viability of target-negative cell lines was observed. Moreover, B-cell depletion was observed in the autologous system in presence of C923B169. T-cell activation with lower cytokine secretion were observed across the in vitro T-cell cytotoxicity assays as compared to the trispecific C923B168, which has medium affinity to CD3, and mosunetuzumab. This is in line with the weak engagement of CD3 by C923B169.

In vivo, C923B169 prevented tumor growth in the CARNAVAL xenograft model and induced potent tumor regression in the OCI-Ly10 xenograft model.

### Example 10: Minimal Target-Related Risks Associated with CD79bxCD20xCD3 Trispecific Antibody

### Normal Tissue Expression

With T-cell-engaging antibodies, on-target/off-tumor toxicity has been identified as a risk in cases where expression of the targeted tumor antigen(s) is not restricted to the tumor. Data in the public domain on CD79b- and CD20-related immune cell and tissue expression were reviewed (results further described below). Furthermore, CD79b expression using IHC on FFPE tissue microarrays (TMAs) was also assessed and CD79b positivity was found to be limited to mononuclear cells consistent with lymphocytes. IHC for CD20 expression was not conducted due to the consistent view from available clinical data for multiple marketed CD20-targeted cytolytic therapeutics.

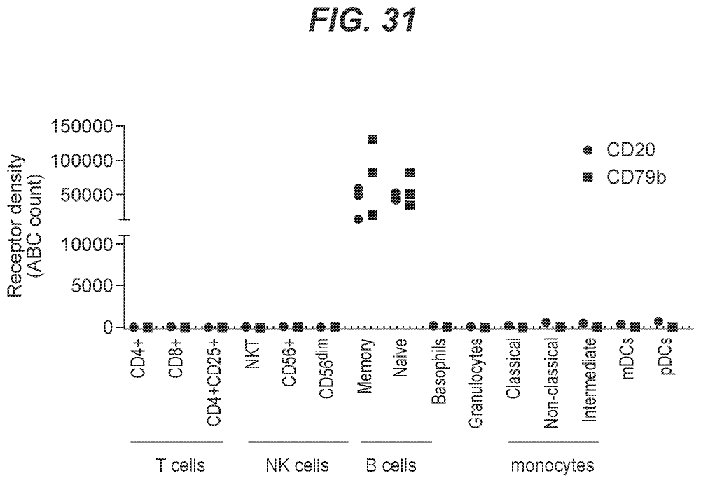

By IHC, CD79b positivity was found to be limited to mononuclear cells consistent with lymphocytes. To confirm B-cell-lineage-specific expression of CD79b and CD20, receptor density was measured on peripheral blood cells derived from 3 different healthy human donors (FIG. 31). The results confirmed that CD79b and CD20 expression are detectable only on B cells as already suggested by the RNA sequencing (RNA-seq) analysis of CD79b performed on 21 distinct purified immune cell subsets isolated from individual peripheral blood samples.

Based on these data, there is a low risk of on-target/off-tumor toxicity for C923B169 beyond B-cell aplasia due to the restricted expression of CD79b and/or CD20 on cells of the B-cell lineage.

### CD79b Tissue Expression

CD79 is a disulfide-linked heterodimer of CD79a and CD79b and constitutes the signaling component of the BCR (19). CD79b is restrictively expressed on the surface of B cells from pre-B to memory B-cell stage and is overexpressed in DLBCL, FL, and MCL, and numerous other B-cell neoplasms (37).

A comprehensive in silico evaluation of CD79b tissue expression was performed based on public and proprietary database and literature searches. CD79b mRNA expression was detected in lymphoid organs, gastrointestinal, immune stem and progenitor cells, and myeloid cells. CD79b protein expression was observed in bone marrow, lymph nodes, spleen, and tonsil (using RNA-seq [CAGE; FANTOM5; Blueprint; Human Proteome Map]). Literature indicates the presence of CD79b-positive staining for most B cells present in peripheral blood and lymphoid tissue (38-40). Although there is 1 literature report of aberrant CD79b T-cell reactivity (41), CD79b protein expression on T cells was not detected by in-house in silico analysis or flow cytometry. In fact, receptor density on non-B immune cells appeared negligible/absent (FIG. 31).

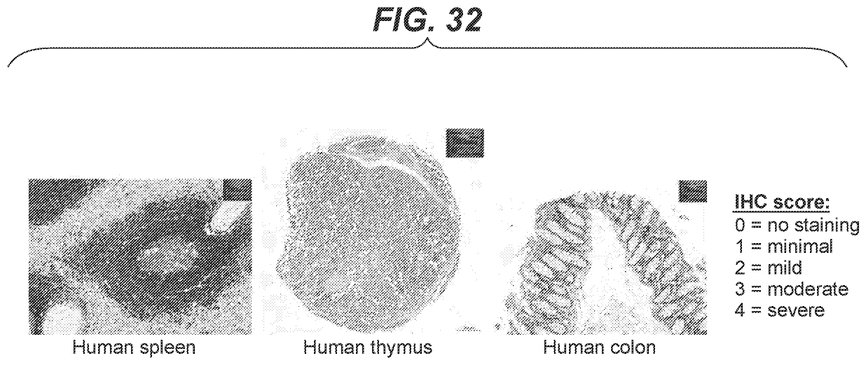

In an IHC study using core biopsies from FFPE human normal TMA, CD79b⁺ staining was restricted to mononuclear cells consistent with B cells (FIG. 32). The distribution pattern of CD79b⁺ IHC-positive cells within tissues was consistent with B cells based on positive cells located primarily in follicular structures in lymph node, spleen, and tonsil. CD79b⁺ mononuclear cell staining in other tissues (ie, small intestine, colon, thymus, bone marrow, breast, lung, esophagus, salivary gland, uterus, cervix, skin) was consistent with resident lymphocytes or lymphoid infiltrates. Non-B-cell subsets of lymphocytes or mononuclear cells cannot be excluded based solely on distribution patterns. However, in peripheral blood, only B cells were CD79b⁺ (FIG. 31).

Similar results were achieved upon IHC staining of an FFPE cynomolgus monkey normal tissue TMA. Detection of mRNA or protein (Human Proteome Map) in tissues is consistent with the presence of resident or circulating B-cell populations as demonstrated by the IHC on human normal TMA. Detection of mRNA on myeloid cells was not demonstrated at the protein level based on the peripheral blood immunophenotyping.

### CD20 Tissue Expression

CD20 is a transmembrane protein restricted to the surface of B cells. Like CD79b, a comprehensive in silico evaluation of CD20 tissue expression analysis was performed. The results for CD20 are similar to CD79b with presence of CD20 mRNA restricted to B cells in lymphoid organs, gastrointestinal (ileum, stomach mucosa, rectum), gall bladder and selected T cells (using RNA-seq [CAGE; FANTOM5; Blueprint; Human Proteome Map]). Literature indicates the presence of CD20⁺ staining for most B cells present in peripheral blood and lymphoid tissue (38-40).

Determination of expression of CD20 was not performed using in-house IHC given the available literature regarding CD20 B-cell-restricted expression in normal tissue (42-45), and over a decade of anti-CD20 cytolytic therapy in numerous cancers and other diseases including NHL, Burkitt lymphoma, FL, CLL (46), relapsing multiple sclerosis, systemic lupus erythematosus, rheumatoid arthritis, and others (47), have not identified on-target/off-tumor toxicity beyond the B-cell lineage. In addition, T-cell depletion has not been reported with B-cell cytolytic therapies. Furthermore, treatment of cynomolgus monkeys with rituximab was well tolerated and exhibited depletion of peripheral blood B cells (48); and knockout of CD20 in the mouse did not impact B-cell development or function (49).

Related to expression on T cells, limited expression of CD20 on memory myelin-specific CD8⁺ T-cells has been directly implicated in multiple sclerosis pathogenesis in addition to its role in B cells. Notably, reconstitution of CD20⁺ T cells occurs more rapidly than CD20⁺ B cells (50,51).

It can be concluded, based upon the information detailed above, that any on-target/off-tumor toxicity for C923B169 is expected to be limited to normal B cells.

### Example 11: Binding Characteristics of the CD3, CD79b and CD20 Binding Arms

Detailed below are the evaluations conducted to determine binding characteristics of the CD3 binding arm and the TAA (i.e., CD79b and CD20) binding arms.

### CD3 Binding Arm

The CD3 binder used in C923B169, CD3B2030, binds to CD3 on the surface of human, but not of cynomolgus monkey, peripheral blood T cells.

CD3B2030 (tested as a NullxCD3 or NullxNullxCD3, 79C3B615 and C923B175, respectively) was assessed by flow cytometry for binding to cynomolgus monkey and human peripheral blood T cells. The results indicated that CD3B2030 does not bind cynomolgus monkey T cells unlike the positive control anti-CD3 clone SP34, which is cross-reactive with cynomolgus monkey CD3. As expected, CD3B2030 did bind to human peripheral blood T cells at a similar frequency as the positive control.

### CD79b Binding Arm

The CD79b-targeting arm demonstrated low (equilibrium dissociation constant [K_{D}] estimated >3 μM) binding to the long CD79b isoform for the cynomolgus monkey protein representing >4-log weaker affinity compared to human, while no binding was observed to mouse CD79b by SPR (Table 32).

As detailed in Table 46, CD79b amino acid percentage identity and similarity across orthologue species is moderately conserved across mammalian species. The low binding/lack of binding is in accordance with the low sequence alignment between human and these nonclinical species at the targeted domain.

TABLE 46
CD79b extracellular domain (ECD) percent identity and
similarity across orthologue species.

| | | | | Cyno- | | | | |
|---|---|---|---|---|---|---|---|---|
| | | | Rhesus | molgus | | | | Guinea |
| | Mouse | Rat | monkey | monkey | Marmoset | Dog | Rabbit | pig |
| Identity | 53.3 | 53.0 | 76.7 | 76.7 | 70.5 | 66.4 | 52.9 | 63.0 |
| Similarity | 71.4 | 73.3 | 85.7 | 85.7 | 80.3 | 78.6 | 69.9 | 78.5 |

These results are similar to polatuzumab vedotin, which was also not cross-reactive to CD79b despite only 3 amino acid difference at the binding epitope between the human and cynomolgus monkey (61).

### CD20 Binding Arm

In a flow cytometry study with a CD20 binder (i.e., C20B680 LH scFv, tested as the IgG1 Fc fusion protein C923B192), binding was observed to both human and cynomolgus monkey B cells. Additionally, C20B648 bound to a similar frequency of cell surface CD20 on human and cynomolgus monkey B cells, further confirming cross-reactivity to cynomolgus monkeys.

As detailed in Table 47, CD20 amino acid percentage identity of the ECD across orthologue species is moderately to highly conserved across mammalian species. The high degree of binding as assessed by flow cytometry to non-human primates is in accordance with the high sequence alignment between human and these nonclinical species at the targeted domain.

TABLE 47
CD20 extracellular domain (ECD) percent identity matrix across
orthologue species.

| | | | | Cyno- | | | | |
|---|---|---|---|---|---|---|---|---|
| | | | Rhesus | molgus | | | | Guinea |
| | Mouse | Rat | monkey | monkey | Marmoset | Dog | Rabbit | pig |
| Identity | 68.9 | 68.9 | 95.6 | 97.8 | 86.7 | 68.9 | 80.0 | 71.1 |

The biophysical assessment for the CD20 binding arm indicated little to no measurable affinity to recombinant human CD20 as full binding curve could not be generated.

### Example 12: Potential C923B169 Off-Target Toxicity

CD9B374 has a low risk for off-target effects based on selective binding and functional activity. There are 2 splice isoforms of CD79b (FIG. 9B), with the long isoform being the prominent form in DLBCL. Specific to the CD79b counterpart in the B-cell receptor, CD79a, low homology exists between the ECD binding regions (Table 47).

To characterize the potential off-target binding of the tumor-antigen-targeting arms of C923B169, namely, CD9B374 (anti-CD79b mAb) and C20B648 LH scFv (anti-CD20 scFv, tested as an Fc fusion, C923B192), the bivalent molecules were evaluated for binding on a human plasma membrane protein array (Retrogenix) in 2 separate non-GLP studies.

### Assessment for Off-Target Binding

### CD79b

The CD79b binder (CD9B374, IgG1) when screened for binding against fixed HEK293 cells individually expressing a library of 5,475 full-length human plasma membrane and cell-surface-tethered human secreted proteins and 371 heterodimers, was determined to bind specifically to its primary target, CD79b with strong intensity when expressed alone or as part of a heterodimer with CD79a. CD9B374 did not bind CD79a expressed alone on HEK293 cells. No off-target interactions were identified, demonstrating the target specificity of the CD79b binding domain contained in C923B169.

### CD20

The CD20 binder (C20B680 LH scFv, tested as an IgG1 Fc fusion protein, C923B192) when screened for binding against fixed HEK293 cells individually expressing a library of 5,681 full-length human plasma membrane and cell-surface-tethered human secreted proteins and 371 heterodimers, was determined to bind specifically to its primary target, CD20, with strong intensity. No off-target interactions were identified, demonstrating the target specificity of the CD20 binding domain contained in C923B169.

### Assessment for Off-Target Functional Activity

### Functional Selectivity of C923B169 in TAA⁻ Cell Lines

The antigen specificity of C923B169 was further characterized in an in vitro functional assay using a panel of 6 cancer cell lines that lack expression of CD79b and CD20, but by transcriptomics are predicted to express >50% of the known cell surface proteins. In coculture studies with healthy donor-derived T cells, C923B169 was able to induce antibody-dependent, T-cell-mediated cytokine release of granzyme B, interferon (IFN)-γ, tumor necrosis factor (TNF)-α, and IL-2 when added to cocultures with target cells that express CD79b and CD20 (i.e., CARNAVAL) but no release (or negligible levels) with cell lines that do not express the target antigens. These data support the antigen specificity of C923B169 to CD79b and CD20 and demonstrate a lack of off-target T-cell activation, as measured by cytokine release.

### Example 13: Pharmacokinetics in Preclinical Species

### Pharmacokinetics in Mouse Efficacious Model

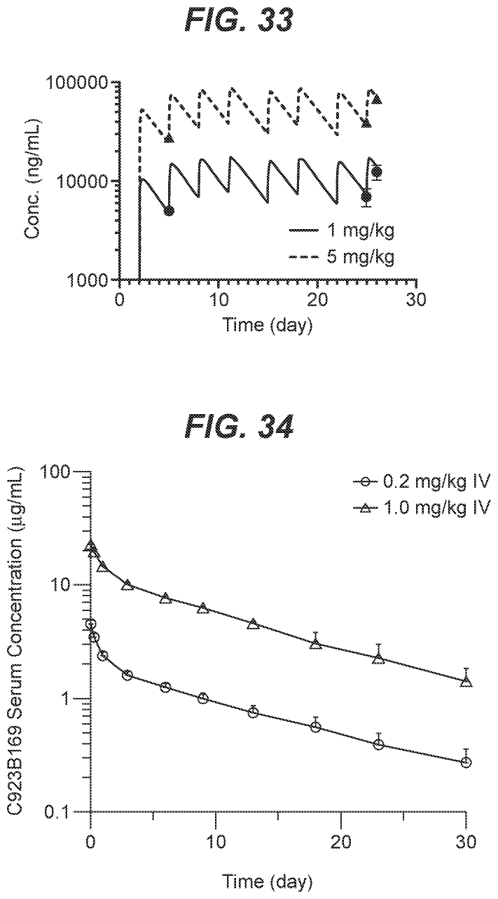

Serum samples were collected from NSG mice bearing SC tumor of the CARNAVAL cell line. Serum concentrations of C923B169 at 72 hours post first dose, 1 hour before and 24 hours post the final dose (Study A) following every 3 or 4 days dosing were fitted using a 2-compartment model to characterize the mouse PK. PK parameters (i.e., plasma clearance [CL], volume of the central compartment [Vc], and volume of the peripheral compartment [Vp]) were estimated for C923B169 by fixing the distribution clearance (Q) to an experience value obtained from a previous study. Data analysis was performed using Monolix 2018R2 (Lixoft®). Model fitting is shown in FIG. 33. Mild serum drug accumulation was observed in mice following repeated dosing at 1 or 5 mg/kg. The observed plasma concentration immediately prior to next administration (C_{trough}) was approximately 7,000 and 40,000 ng/mL at 1 and 5 mg/kg, respectively. The results are used in PK/PD correlation in the mouse models.

### Pharmacokinetics in Monkey and Minipig

The PK of C923B169 is being studied in cynomolgus monkeys and Göttingen minipigs.

In the monkey study, C923B169 was administered as a single dose at 0.2 and 1.0 mg/kg intravenously (IV; as 0.2 and 1 mg/mL in 10 mM histidine, pH 6.5 dosing formulations). Serum samples were collected for 30 days. Preliminary PK results in cyno monkeys are shown in FIG. 34 and Table 48.

TABLE 48
Summary of PK results in cyno monkeys

| | | | AUCₗₐₛₜ | AUC_{inf} | CL | | |
|---|---|---|---|---|---|---|---|
| | | Cₘₐₓ | (μg · | (μg · | (mL/ | V_{z} | |
| | | (μg/ | day/ | day/ | day/ | (mL/ | T_{1/2} |
| C923B169 | N = 3 | mL) | mL) | mL) | kg) | kg) | (day) |
| 0.2 mg/kg | Mean | 4.51 | 26.04 | 30.64 | 6.66 | 109.73 | 11.59 |
| IV | SD | 0.42 | 3.39 | 5.46 | 1.10 | 11.28 | 1.82 |
| 1.0 mg/kg | Mean | 22.88 | 156.21 | 178.70 | 5.65 | 88.07 | 10.83 |
| IV | SD | 1.54 | 15.24 | 22.88 | 0.68 | 9.69 | 0.80 |

In the minipig study, C923B169 was administered as a single dose at 1.0 mg/kg IV or SC (as 3.39 mg/mL in 10 mM histidine, pH 6.5 dosing formulation) to four male minipigs per study group. The dose was given on study Day 1. Serum samples are collected for up to 45 days. For quantification of C923B169 concentrations, the lowest quantifiable concentration in a sample was 0.08 μg/mL (lower limit of quantification×minimum required dilution).

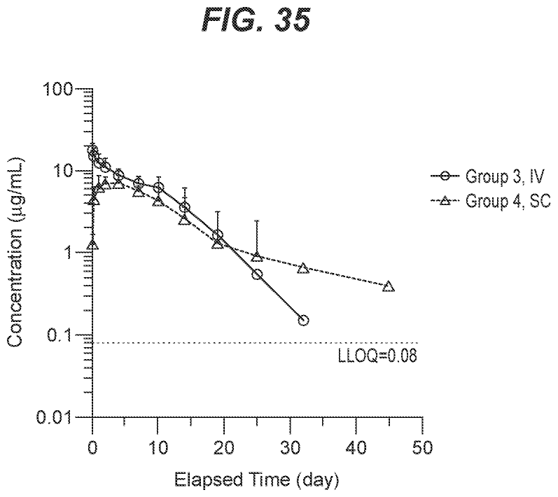

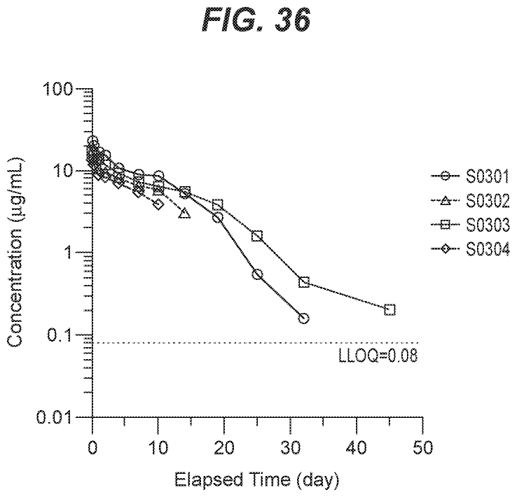

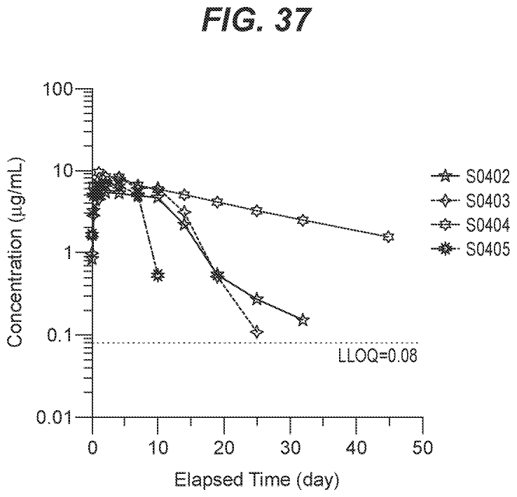

Individual and mean (SD) PK parameters of C923B169 following single IV or SC dose are summarized in Table 49. Mean (SD) serum concentrations of C923B169 versus time for male minipigs are illustrated in FIG. 35. Individual serum concentrations of C923B169 versus time for male minipigs are illustrated in FIG. 36 and FIG. 37.

TABLE 49
Individual and mean (SD) serum C923B169 PK parameters following a
single IV or SC dose of C923B169 at 1 mg/kg in male minipigs

| | | | | AUCₗₐₛₜ | | AUC_{inf} | CL | | |
|---|---|---|---|---|---|---|---|---|---|
| | | Cₘₐₓ | | (μg · | | (μg · | (mL/ | V_{z} | |
| | | (μg/ | Tₘₐₓ | day/ | Tₗₐₛₜ | day/ | day/ | (mL/ | T_{1/2} |
| Group | ID | mL) | (day) | mL) | (day) | mL) | kg) | kg) | (day) |
| Group 3 | S0301 | 22.59 | — | 174.02 | 32.00 | 204.48 | 4.89 | 78.00 | 11.06 |
| IV | S0302 | 17.56 | — | 98.23 | 14.00 | 184.40^{a} | 5.42 | 100.73 | 12.87 |
| | S0303 | 16.70 | — | 166.74 | 45.00 | 188.25 | 5.31 | 105.01 | 13.70 |
| | S0304 | 13.54 | — | 66.64 | 10.00 | 109.94^{a} | 9.10 | 98.82 | 7.53 |
| | Mean | 17.60 | — | 126.41 | 25.25 | 171.77 | 6.18 | 95.64 | 11.29 |
| | SD | 3.75 | — | 52.47 | 16.28 | 42.13 | 1.96 | 12.04 | 2.74 |
| Group 4 | S0402 | 5.39 | 2.00 | 71.50 | 32.00 | 78.59 | — | — | 8.36 |
| SC | S0403 | 8.27 | 4.00 | 90.38 | 25.00 | 102.10 | — | — | 7.04 |
| | S0404 | 9.50 | 1.00 | 186.17 | 45.00 | 228.60 | — | — | 18.87 |
| | S0405 | 7.01 | 2.00 | 49.23 | 10.00 | NR^{b} | — | — | NR^{b} |
| | Mean | 7.54 | 2.25 | 99.32 | 28.00 | 136.43 | — | — | 11.42 |
| | SD | 1.76 | 1.26 | 60.29 | 14.58 | 80.68 | — | — | 6.49 |

^{a}Extrapolated AUC was larger than 20% for all animals.
^{b}Not applicable due to poorly characterized terminal phase and has been excluded from the calculation of the mean data.

Serum C923B169 concentrations were below the lowest quantifiable concentration in all samples collected prior to the dose administration in C923B169-treated IV and SC groups.

Following a single IV administration of C923B169, serum C923B169 concentrations decreased from the first post-dose collection (0.04 day) through Tₗₐₛₜ (14 or 32 days post-dose). Mean clearance, volume of distribution and terminal half-life following single IV administration of C923B169 at 1 mg/kg was found to be 6.18 mL/kg/day, 95.64 mL/kg, and 11.29 days, respectively.

Following a single SC administration of C923B169, serum C923B169 concentrations were quantifiable from 0.04 to 45 days post-dose. Mean serum C923B169 concentrations reached the Cₘₐₓ at 1-4 days post-dose and then decreased through Tₗₐₛₜ. Terminal half-life following single SC administration C923B169 at 1 mg/kg was estimated to be 11.42 days and was comparable to that of IV administration (11.29 days) at similar dose level. The SC bioavailability was estimated as 79% by comparing AUC_{inf} following a single SC and IV dose.

An aberrant decrease in serum C923B169 concentrations were observed in most of the animals from the IV and SC dosing groups, which were likely due to the impact of anti-drug antibodies (ADA); however, ADA was not assayed in the study.

The quantifiable serum C923B169 concentrations that were likely impacted by ADA development were not used in the calculation of AUC_{inf}, CL, V_{z} and T_{1/2} (Table 50 and Table 51).

TABLE 50
Individual and mean (SD) serum C923B169 concentrations
(μg/ml) following a single IV dose of 1 mg/kg
of C923B169 in male minipigs (Group 3)

| Protocol | Time | | | | | | |
|---|---|---|---|---|---|---|---|
| Time | (day) | S0301 | S0302 | S0303 | S0304 | Mean | SD |
| Predose | 0 | <0.08 | <0.08 | <0.08 | <0.08 | <0.08 | NR |
| 1 hr | 0.04 | 22.59 | 17.56 | 16.70 | 13.54 | 17.60 | 3.75 |
| 6 hr | 0.25 | 20.03 | 13.62 | 15.28 | 11.28 | 15.05 | 3.70 |
| 24 hr | 1 | 16.42 | 10.60 | 13.97 | 8.73 | 12.43 | 3.43 |
| 48 hr | 2 | 15.26 | 9.42 | 11.51 | 8.11 | 11.08 | 3.12 |
| 96 hr | 4 | 10.58 | 8.18 | 9.32 | 7.03 | 8.78 | 1.52 |
| 168 hr | 7 | 8.97 | 6.39 | 7.18 | 5.45 | 7.00 | 1.49 |
| 240 hr | 10 | 8.76 | 5.92 | 6.42 | 3.88 | 6.24 | 2.01 |
| 336 hr | 14 | 5.45 | 3.11* | 5.64 | <0.08 | 3.55 | 2.63 |
| 456 hr | 19 | 2.69* | <0.08 | 3.87 | <0.08 | 1.64 | NR |
| 600 hr | 25 | 0.55* | <0.08 | 1.60* | <0.08 | 0.54 | NR |
| 768 hr | 32 | 0.16* | <0.08 | 0.44* | <0.08 | 0.15 | NR |
| 1080 hr | 45 | <0.08 | <0.08 | 0.20* | <0.08 | <0.08 | NR |

*The time points were not used for the calculation of AUC_{inf}, CL, V_{z} and T_{1/2} due to possible ADA impact.

TABLE 51
Individual and mean (SD) serum C923B169 concentrations
(μg/mL) following a single SC dose of 1 mg/kg of
C923B169 in male minipigs (Group 4)

| Protocol | Time | | | | | | |
|---|---|---|---|---|---|---|---|
| Time | (day) | S0402 | S0403 | S0404 | S0405 | Mean | SD |
| Predose | 0 | <0.08 | <0.08 | <0.08 | <0.08 | <0.08 | <0.08 |
| 1 hr | 0.04 | 0.84 | 0.98 | 1.60 | 1.67 | 1.27 | 0.42 |
| 6 hr | 0.25 | 3.24 | 2.81 | 6.76 | 5.14 | 4.49 | 1.82 |
| 24 hr | 1 | 4.74 | 4.38 | 9.50 | 6.84 | 6.36 | 2.35 |
| 48 hr | 2 | 5.39 | 6.92 | 8.47 | 7.01 | 6.95 | 1.26 |
| 96 hr | 4 | 5.27 | 8.27 | 8.24 | 6.51 | 7.07 | 1.45 |
| 168 hr | 7 | 4.82 | 6.06 | 6.70 | 5.18 | 5.69 | 0.85 |
| 240 hr | 10 | 4.68 | 6.19 | 5.90 | 0.53 | 4.32 | 2.62 |
| 336 hr | 14 | 2.22 | 3.13 | 5.07 | <0.08 | 2.60 | 2.10 |
| 456 hr | 19 | 0.55* | 0.51* | 4.09 | <0.08 | 1.29 | 1.89 |
| 600 hr | 25 | 0.27* | 0.11* | 3.24 | <0.08 | 0.90 | 1.56 |
| 768 hr | 32 | 0.15* | <0.08 | 2.47 | <0.08 | 0.66 | NR |
| 1080 hr | 45 | <0.08 | <0.08 | 1.57 | <0.08 | 0.39 | NR |

*The time points were not used for the calculation of AUC_{inf}, CL, V_{z} and T_{1/2} due to possible ADA impact.

The ADA development may impact the evaluation of bioavailability; however the extent of impact could not be concluded.

### Pharmacokinetic/Pharmacodynamic Analysis

A full characterization of the in vitro and in vivo PK/PD relationship of C923B169 is performed using relevant in vitro potency data and in vivo dose-efficacy relationship from the mouse efficacy model. The PK results from the ongoing PK and SC bioavailability evaluation in monkeys and minipigs are the basis of human efficacious dose projection.

### REFERENCES

1. Kochenderfer, J. N. et al. Chemotherapy-refractory diffuse large B-cell lymphoma and indolent B-cell malignancies can be effectively treated with autologous T cells expressing an anti-CD19 chimeric antigen receptor. J Clin Oncol 33, 540-549 (2015).
2. Schuster, S. J. et al. Chimeric Antigen Receptor T Cells in Refractory B-Cell Lymphomas. N Engl J Med 377, 2545-2554 (2017).
3. Shalabi, H. et al. Sequential loss of tumor surface antigens following chimeric antigen receptor T-cell therapies in diffuse large B-cell lymphoma. Haematologica 103, e215-e218 (2018).
4. Sotillo, E. et al. Convergence of Acquired Mutations and Alternative Splicing of CD19 Enables Resistance to CART-19 Immunotherapy. Cancer Discov 5, 1282-1295 (2015).
5. Packard, T. A. & Cambier, J. C. B lymphocyte antigen receptor signaling: initiation, amplification, and regulation. F1000Prime Rep 5, 40 (2013).
6. Puri, K. D., Di Paolo, J. A. & Gold, M. R. B-cell receptor signaling inhibitors for treatment of autoimmune inflammatory diseases and B-cell malignancies. Int Rev Immunol 32, 397-427 (2013).
7. Polson, A. G. et al. Antibody-drug conjugates targeted to CD79 for the treatment of non-Hodgkin lymphoma. Blood 110, 616-623 (2007).
8. Palanca-Wessels, M. C. A. et al. Safety and activity of the anti-CD79B antibody-drug conjugate polatuzumab vedotin in relapsed or refractory B-cell non-Hodgkin lymphoma and chronic lymphocytic leukaemia: a phase 1 study. The Lancet Oncology 16, 704-715 (2015).
9. Astsaturov, I. A. et al. Differential expression of B29 (CD79b) and mb-1 (CD79a) proteins in acute lymphoblastic leukaemia. Leukemia 10, 769-773 (1996).
10. Foster L H, Lum L G. Treatment of hematological malignancies with T cell redirected bispecific antibodies: current status and future needs. Expert Opin Biol Ther. 2019; 19(7):707-720. doi:10.1080/14712598.2019.1604672
11. Dufner V, Sayehli C M, Chatterjee M, et al. Long-term outcome of patients with relapsed/refractory B-cell non-Hodgkin lymphoma treated with blinatumomab. Blood Adv. 2019; 3(16):2491-2498. doi:10.1182/bloodadvances.2019000025
12. Burt R, Warcel D, Fielding A K. Blinatumomab, a bispecific B-cell and T-cell engaging antibody, in the treatment of B-cell malignancies. Hum Vaccin Immunother. 2019; 15(3):594-602. doi:10.1080/21645515.2018.1540828
13. Bibikova E, Law B, Clevenger T, et al. High surface expression of CD49d (VLA-4) and CD79b correlates with acalabrutinib resistance in patients with chronic lymphocytic leukemia (CLL). Blood. 2019; 134(Supplement_1):2571. doi: 10.1182/blood-2019-128872
14. Visco C, Tanasi I, Quaglia F M, Ferrarini I, Fraenza C, Krampera M. Oncogenic mutations of MYD88 and CD79b in diffuse large B-cell lymphoma and implications for clinical practice. Cancers (Basel). 2020; 12(10):2913. Published 2020 Oct. 10. doi:10.3390/cancers12102913
15. Davis R E, Ngo V N, Lenz G, et al. Chronic active B-cell-receptor signalling in diffuse large B-cell lymphoma. Nature. 2010; 463(7277):88-92. doi:10.1038/nature08638
16. Phelan J D, Young R M, Webster D E, et al. A multiprotein supercomplex controlling oncogenic signalling in lymphoma. Nature. 2018; 560(7718):387-391. doi:10.1038/s41586-018-0290-0
17. He X, Kläsener K, Iype J M, et al. Continuous signaling of CD79b and CD19 is required for the fitness of Burkitt lymphoma B cells. EMBO J. 2018; 37(11):e97980. doi:10.15252/embj.201797980
18. Cragg M S, Chan H T, Fox M D, et al. The alternative transcript of CD79b is overexpressed in B-CLL and inhibits signaling for apoptosis. Blood. 2002; 100(9):3068-3076. doi:10.1182/blood.V100.9.3068
19. Burger J A, Wiestner A. Targeting B cell receptor signalling in cancer: preclinical and clinical advances. Nat Rev Cancer. 2018; 18(3):148-167. doi:10.1038/nrc.2017.121
20. Klein C, Lammens A, Schafer W, et al. Epitope interactions of monoclonal antibodies targeting CD20 and their relationship to functional properties. MAbs. 2013; 5(1):22-33. doi:10.4161/mabs.22771
21. Scheuplein R J. Permeability of the skin: a review of major concepts. Curr Probl Dermatol. 1978; 7:172-186. doi:10.1159/000401285
22. Eon Kuek L, Leffler M, Mackay G A, Hulett M D. The MS4A family: counting past 1, 2 and 3. Immunol Cell Biol. 2016; 94(1):11-23. doi:10.1038/icb.2015.48
23. Gamonet C, Bole-Richard E, Delherme A, et al. New CD20 alternative splice variants: molecular identification and differential expression within hematological B cell malignancies [published correction appears in Exp Hematol Oncol. 2015; 5:10]. Exp Hematol Oncol. 2016; 5:7. Published 2016 Mar. 1. doi:10.1186/s40164-016-0036-3
24. Henry C, Deschamps M, Rohrlich P S, et al. Identification of an alternative CD20 transcript variant in B-cell malignancies coding for a novel protein associated to rituximab resistance. Blood. 2010; 115(12):2420-2429. doi:10.1182/blood-2009-06-229112
25. Tedder T F, Schlossman S F. Phosphorylation of the B1 (CD20) molecule by normal and malignant human B lymphocytes. J Biol Chem. 1988; 263(20):10009-10015.
26. Bubien J K, Zhou L J, Bell P D, Frizzell R A, Tedder T F. Transfection of the CD20 cell surface molecule into ectopic cell types generates a Ca2+ conductance found constitutively in B lymphocytes. J Cell Biol. 1993; 121(5):1121-1132. doi:10.1083/jcb.121.5.1121
27. Brouwer-Visser J, Fiaschi N, Deering R P, et al. Baseline biomarkers of T-cell function correlate with clinical responses to odronextamab (REGN1979), and loss of CD20 target antigen expression identified as a mechanism of treatment resistance. 62^{nd} ASH Annual Meeting and Exposition. Dec. 5-8, 2020; Abstract 2108. Available from: https://ash.confex.com/ash/2020/webprogram/Paper137499.html. Accessed: 3 Jun. 2021.
28. Salvaris R, Ong J, Gregory G P. Bispecific Antibodies: A review of development, clinical efficacy and toxicity in B-cell lymphomas. J Pers Med. 2021; 11(5):355. Published 2021 Apr. 29. doi:10.3390/jpml1050355
29. American Cancer Society. Non-Hodgkin lymphoma [internet]. Atlanta, G A: ACS; 2018. Available from: https://www.cancer.org/cancer/non-hodgkin-lymphoma/about/key-statistics.html. Accessed: 17 May 2021.
30. Evans A G, Rothberg P G, Burack W R, et al. Evolution to plasmablastic lymphoma evades CD19-directed chimeric antigen receptor T cells. Br J Haematol. 2015; 171(2):205-209. doi:10.111l/bjh.13562
31. Porter D L, Levine B L, Kalos M, Bagg A, June C H. Chimeric antigen receptor-modified T cells in chronic lymphoid leukemia [published correction appears in N Engl J Med. 2016 Mar. 10; 374(10):998]. N Engl J Med. 2011; 365(8):725-733. doi:10.1056/NEJMoa1103849
32. Shalabi H, Kraft I L, Wang H W, et al. Sequential loss of tumor surface antigens following chimeric antigen receptor T-cell therapies in diffuse large B-cell lymphoma. Haematologica. 2018; 103(5):e215-e218. doi:10.3324/haematol.2017.183459
33. Fry T J, Shah N N, Orentas R J, et al. CD22-targeted CAR T cells induce remission in B-ALL that is naive or resistant to CD19-targeted CAR immunotherapy. Nat Med. 2018; 24(1):20-28. doi:10.1038/nm.4441
34. Majzner R G, Mackall C L. Tumor antigen escape from CAR T-cell therapy. Cancer Discov. 2018; 8(10):1219-1226. doi:10.1158/2159-8290.CD-18-0442
35. Decision Resource Group. Non-Hodgkin's lymphoma and chronic lymphocytic leukemia. Burlington, M A: Decision Resources Group; November 2019.
36. Ito M, Zhao N, Zeng Z, Zhou X, Chang C C, Zu Y. Interleukin-2 functions in anaplastic large cell lymphoma cells through augmentation of extracellular signal-regulated kinases 1/2 activation. Int J Biomed Sci. 2011; 7(3):181-190.
37. Chu P G, Arber D A. CD79: a review. Appl Immunohistochem Mol Morphol. 2001; 9(2):97-106. doi:10.1097/00129039-200106000-00001
38. Guisado Vasco P, Villar Rodríguez J L, Ibañez Martínez J, González Cámpora R, Galera Davidson H. Immunohistochemical organization patterns of the follicular dendritic cells, myofibroblasts and macrophages in the human spleen—new considerations on the pathological diagnosis of splenectomy pieces. Int J Clin Exp Pathol. 2009; 3(2):189-202. Published 2009 Dec. 10.
39. Tomita A. Genetic and Epigenetic Modulation of CD20 Expression in B-Cell Malignancies: molecular mechanisms and significance to rituximab resistance. J Clin Exp Hematop. 2016; 56(2):89-99. doi:10.3960/jslrt.56.89
40. Zuccolo J, Deng L, Unruh T L, et al. Expression of MS4A and TMEM176 genes in human B lymphocytes. Front Immunol. 2013; 4:195. Published 2013 Jul. 15. doi:10.3389/fimmu.2013.00195
41. Jahn L, Hombrink P, Hassan C, et al. Therapeutic targeting of the BCR-associated protein CD79b in a TCR-based approach is hampered by aberrant expression of CD79b. Blood. 2015; 125(6):949-958. doi:10.1182/blood-2014-07-587840
42. Middleton O, Wheadon H, Michie A. Classical complement pathway. In: Ratcliffe M J H, ed. Encyclopedia of immunobiology, 1st ed. New York, N Y: Elsevier; 2016: Volume 2. doi:10.1016/B978-0-12-374279-7.02014-2
43. Ajina R, et al. Cancer Immunotherapy. In: Chackalamannil S, Rotella D, Ward S, eds. Comprehensive medicinal chemistry III, 3′ ed. New York, NY: Elsevier; 2017: Volume 6. doi:10.1016/B978-0-12-409547-2.12426-62017
44. Naeim F, Rao P N, Song S X, Phan R T. Chapter 2: principles of Immunophenotyping. In: Naeim F, Rao P N, Song S X, Phan R T, eds. Atlas of hematopathology: morphology, immunophenotype, cytogenetics, and molecular approaches, 2nd ed. Cambridge, M A: Academic Press; 2018. doi:10.1016/B978-0-12-809843-1.00002-4
45. Uchida J, Lee Y, Hasegawa M, et al. Mouse CD20 expression and function. Int Immunol. 2004; 16(1):119-129. doi:10.1093/intimm/dxh009
46. Kläsener K, Jellusova J, Andrieux G, et al. CD20 as a gatekeeper of the resting state of human B cells. Proc Natl Acad Sci USA. 2021; 118(7):e2021342118. doi:10.1073/pnas.2021342118
47. Lee D S W, Rojas O L, Gommerman J L. B cell depletion therapies in autoimmune disease: advances and mechanistic insights. Nat Rev Drug Discov. 2021; 20(3):179-199. doi:10.1038/s41573-020-00092-2
48. Schröder C, Azimzadeh A M, Wu G, Price J O, Atkinson J B, Pierson R N. Anti-CD20 treatment depletes B-cells in blood and lymphatic tissue of cynomolgus monkeys. Transpl Immunol. 2003; 12(1):19-28. doi:10.1016/S0966-3274(03)00059-5
49. O'Keefe T L, Williams G T, Davies S L, Neuberger M S. Mice carrying a CD20 gene disruption. Immunogenetics. 1998; 48(2):125-132. doi:10.1007/s002510050412
50. Sabatino J J Jr, Wilson M R, Calabresi P A, Hauser S L, Schneck J P, Zamvil S S. Anti-CD20 therapy depletes activated myelin-specific CD8⁺ T cells in multiple sclerosis. Proc Natl Acad Sci USA. 2019; 116(51):25800-25807. doi:10.1073/pnas.1915309116
51. Waisman A, Ebering A. Unraveling the T-B tangle in anti-CD20 multiple sclerosis therapy. Proc Natl Acad Sci USA. 2019; 116(51):25376-25377. doi:10.1073/pnas.1919044116
52. Lu T, Gibiansky L, Li X, et al. Exposure-safety and exposure-efficacy analyses of polatuzumab vedotin in patients with relapsed or refractory diffuse large B-cell lymphoma. Leuk Lymphoma. 2020; 61(12):2905-2914. doi:10.1080/10428194.2020.1795154
53. Arcari A, Chiappella A, Spina M, et al. Safety and efficacy of rituximab plus bendamustine in relapsed or refractory diffuse large B-cell lymphoma patients: an Italian retrospective multicenter study. Leuk Lymphoma. 2016; 57(8):1823-1830. doi:10.3109/10428194.2015.1106536
54. Gea-Banacloche J C. Rituximab-associated infections. Semin Hematol. 2010; 47(2):187-198. doi:10.1053/j.seminhematol.2010.01.002
55. Sacco K A, Abraham R S. Consequences of B-cell-depleting therapy: hypogammaglobulinemia and impaired B-cell reconstitution. Immunotherapy. 2018; 10(8):713-728. doi:10.2217/imt-2017-0178
56. Kelesidis T, Daikos G, Boumpas D, Tsiodras S. Does rituximab increase the incidence of infectious complications?A narrative review. Int J Infect Dis. 2011; 15(1):e2-e16. doi:10.1016/j.ijid.2010.03.025
57. Kusumoto S, Arcaini L, Hong X, et al. Risk of HBV reactivation in patients with B-cell lymphomas receiving obinutuzumab or rituximab immunochemotherapy. Blood. 2019; 133(2):137-146. doi:10.1182/blood-2018-04-848044
58. Klein C, Jamois C, Nielsen T. Anti-CD20 treatment for B-cell malignancies: current status and future directions. Expert Opin Biol Ther. 2021; 21(2):161-181. doi:10.1080/14712598.2020.1822318
59. International Council for Harmonisation of Technical Requirements for Pharmaceuticals for Human Use. ICH S6(R1) Guideline: Preclinical safety evaluation of biotechnology-derived pharmaceuticals; July 1997/Addendum R1 incorporated June 2011 [internet]. Geneva, Switzerland: ICH; 2011. Available from: https://database.ich.org/sites/default/files/S6_R1_Guideline_0.pdf. Accessed: 19 May 2021.
60. Singh A, Dees S, Grewal I S. Overcoming the challenges associated with CD3+ T-cell redirection in cancer. Br J Cancer. 2021; 124(6):1037-1048. doi:10.1038/s41416-020-01225-5
61. Li D, Lee D, Dere R C, et al. Evaluation and use of an anti-cynomolgus monkey CD79b surrogate antibody-drug conjugate to enable clinical development of polatuzumab vedotin. Br J Pharmacol. 2019; 176(19):3805-3818. doi:10.1111/bph.14784
62. Li J, Piskol R, Ybarra R, et al. CD3 bispecific antibody-induced cytokine release is dispensable for cytotoxic T cell activity. Sci Transl Med. 2019; 11(508):eaax8861. doi:10.1126/scitranslmed.aax8861
63. Leong S R, Sukumaran S, Hristopoulos M, et al. An anti-CD3/anti-CLL-1 bispecific antibody for the treatment of acute myeloid leukemia. Blood. 2017; 129(5):609-618. doi:10.1182/blood-2016-08-735365
64. Jain T, Litzow M R. Management of toxicities associated with novel immunotherapy agents in acute lymphoblastic leukemia. Ther Adv Hematol. 2020; 11:2040620719899897. Published 2020 Jan. 20. doi:10.1177/2040620719899897
65. Bargou R, Leo E, Zugmaier G, et al. Tumor regression in cancer patients by very low doses of a T cell-engaging antibody. Science. 2008; 321(5891):974-977. doi:10.1126/science.1158545
66. Klinger M, Brandl C, Zugmaier G, et al. Immunopharmacologic response of patients with B-lineage acute lymphoblastic leukemia to continuous infusion of T cell-engaging CD19/CD3-bispecific BiTE antibody blinatumomab. Blood. 2012; 119(26):6226-6233. doi:10.1182/blood-2012-01-400515
67. Nagele V, Kratzer A, Zugmaier G, et al. Changes in clinical laboratory parameters and pharmacodynamic markers in response to blinatumomab treatment of patients with relapsed/refractory ALL. Exp Hematol Oncol. 2017; 6:14. Published 2017 May 18. doi:10.1186/s40164-017-0074-5
68. Kamphorst A O, Wieland A, Nasti T, et al. Rescue of exhausted CD8 T cells by PD-1-targeted therapies is CD28-dependent. Science. 2017; 355(6332):1423-1427. doi:10.1126/science.aaf0683
69. Verma V, Shrimali R K, Ahmad S, et al. PD-1 blockade in subprimed CD8 cells induces dysfunctional PD-1+CD38hi cells and anti-PD-1 resistance [published correction appears in Nat Immunol. 2019 Sep. 24:]. Nat Immunol. 2019; 20(9):1231-1243. doi:10.1038/s41590-019-0441-y
70. Guo B, Zhang L, Chiorazzi N, Rothstein T L. IL-4 rescues surface IgM expression in chronic lymphocytic leukemia. Blood. 2016; 128(4):553-562. doi:10.1182/blood-2015-11-682997
71. Aguilar-Hernandez M M, Blunt M D, Dobson R, et al. IL-4 enhances expression and function of surface IgM in CLL cells. Blood. 2016; 127(24):3015-3025. doi:10.1182/blood-2015-11-682906
72. Jacoby E, Nguyen S M, Fountaine T J, et al. CD19 CAR immune pressure induces B-precursor acute lymphoblastic leukaemia lineage switch exposing inherent leukaemic plasticity. Nat Commun. 2016; 7:12320. Published 2016 Jul. 27. doi:10.1038/ncommsi2320
73. Köhnke T, Krupka C, Tischer J, Knösel T, Subklewe M. Increase of PD-L1 expressing B-precursor ALL cells in a patient resistant to the CD19/CD3-bispecific T cell engager antibody blinatumomab. J Hematol Oncol. 2015; 8:111. Published 2015 Oct. 8. doi:10.1186/si3045-015-0213-6
74. Kobold S, Pantelyushin S, Rataj F, Vom Berg J. Rationale for combining bispecific T cell activating antibodies with checkpoint blockade for cancer therapy. Front Oncol. 2018; 8:285. Published 2018 Jul. 25. doi:10.3389/fonc.2018.00285

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1-42. (canceled)

43. A method for treating hematological cancer in a subject in need thereof, said method comprising administering to the subject a therapeutically effective amount of a trispecific antibody, or a trispecific binding fragment thereof, or a pharmaceutical composition comprising the same,
wherein the trispecific antibody, or the trispecific binding fragment thereof, comprises:
(a) a first antigen-binding arm comprising a first heavy chain variable domain (VH1) and a first light chain variable domain (VL1), wherein the VH1 and VL1 comprise the HCDR1, the HCDR2, the HCDR3, the LCDR1, the LCDR2, and the LCDR3 of:
a) SEQ ID NOs: 1, 2, 3, 4, 5 and 6, respectively;
b) SEQ ID NOs: 13, 8, 9, 10, 11 and 12, respectively;
c) SEQ ID NOs: 7, 8, 9, 10, 11 and 12, respectively;
d) SEQ ID NOs: 14, 15, 16, 17, 5 and 6, respectively;
e) SEQ ID NOs: 18, 8, 19, 20, 21 and 12, respectively;
f) SEQ ID NOs: 22, 23, 24, 25, 5 and 6, respectively;
g) SEQ ID NOs: 22, 26, 27, 28, 5 and 29, respectively; or
h) SEQ ID NOs: 30, 31, 32, 33, 5 and 6, respectively;
(b) a second antigen-binding arm comprising a second heavy chain variable domain (VH2) and a second light chain variable domain (VL2), wherein the VH2 and VL2 comprise the HCDR1, the HCDR2, the HCDR3, the LCDR1, the LCDR2, and the LCDR3 of:
a) SEQ ID NOs: 76, 77, 78, 79, 80 and 81, respectively;
b) SEQ ID NOs: 76, 77, 75, 79, 80 and 81, respectively;
c) SEQ ID NOs: 76, 77, 82, 79, 80 and 81, respectively; or
d) SEQ ID NOs: 83, 84, 85, 86, 87 and 88, respectively;
(c) a third antigen-binding arm comprising a third heavy chain variable domain (VH3) and a third light chain variable domain (VL3), wherein the VH3 and VL3 comprise the HCDR1, the HCDR2, the HCDR3, the LCDR1, the LCDR2, and the LCDR3 of:
a) SEQ ID NOs: 115, 116, 117, 118, 119 and 120, respectively;
b) SEQ ID NOs: 121, 122, 123, 124, 119 and 125, respectively;
c) SEQ ID NOs: 115, 116, 95, 96, 119 and 125, respectively; or
d) SEQ ID NOs: 121, 116, 123, 124, 119 and 125, respectively,
wherein the first antigen-binding arm binds to an epitope on cluster of differentiation 79B protein (CD79b), the second antigen-binding arm binds to an epitope on cluster of differentiation 3 (CD3), and the third antigen-binding arm binds to an epitope on cluster of differentiation 20 (CD20).

44. The method of claim 43, wherein the hematological cancer is a CD79b and/or CD20-expressing B cell cancer.

45. The method of claim 44, wherein the CD79b and/or CD20-expressing B cell cancer is a B-cell lymphoma or a non-Hodgkin lymphoma.

46. The method of claim 43, wherein the hematological cancer is a diffuse large B-cell lymphoma (DLBCL), mantle cell lymphoma (MCL), marginal zone lymphoma (MZL), follicular lymphoma (FL), chronic lymphocytic leukemia (CLL), or Waldenstrom macroglobulinemia (WM).

47. The method of claim 43, wherein the hematological cancer is relapsed, refractory, or malignant cancer, or any combination thereof.

48. The method of claim 43, further comprising administering a second therapeutic agent.

49. The method of claim 48, wherein the second therapeutic agent is a surgery, chemotherapy, androgen deprivation therapy or radiation, anti-CD20 agent, anti-CD19 agent, anti-CD22 agent, Bruton's tyrosine kinase (BTK) inhibitor, mucosa-associated lymphoid tissue lymphoma translocation protein 1 (MALT1) inhibitor, immunomodulatory imide drug (IMiD), pro apoptotic B cell lymphoma 2 (Bcl-2) family inhibitor, phosphoinositide 3-kinase (PI3K) inhibitor, immune checkpoint inhibitor, CD28 costimulatory bispecific antibody, CD137 costimulatory bispecific antibody, or any combination thereof.

50. The method of claim 43, wherein the trispecific antibody or trispecific binding fragment, or the pharmaceutical composition is administered intravenously, intramuscularly, intraperitoneally, or subcutaneously to the subject.

51. The method of claim 43, wherein the trispecific antibody or trispecific binding fragment, or the pharmaceutical composition is administered subcutaneously to the subject.

52. The method of claim 43, wherein the first antigen-binding arm that binds CD79b comprises the HCDR1, the HCDR2, the HCDR3, the LCDR1, the LCDR2 and the LCDR3 of SEQ ID NOs: 1, 2, 3, 4, 5 and 6, respectively;
the second antigen-binding arm that binds CD3 comprises the HCDR1, the HCDR2, the HCDR3, the LCDR1, the LCDR2 and the LCDR3 of SEQ ID NOs: 76, 77, 75, 79, 80 and 81, respectively; and
the third antigen-binding arm that binds CD20 comprises the HCDR1, the HCDR2, the HCDR3, the LCDR1, the LCDR2 and the LCDR3 of SEQ ID NOs: 121, 122, 123, 124, 119 and 125, respectively.

53. The method of claim 43, wherein the first antigen-binding arm that binds CD79b comprises the VH1 of SEQ ID NO: 35 and the VL1 of SEQ ID NO: 37;
the second antigen-binding arm that binds CD3 comprises the VH2 of SEQ ID NO: 101 and the VL2 of SEQ ID NO: 99; and
the third antigen-binding arm that binds CD20 comprises the VH3 of SEQ ID NO: 130 and the VL3 of SEQ ID NO: 132.

54. A method for inhibiting growth or proliferation of a cancer cell in a subject, said method comprising administering to said subject an effective amount of a trispecific antibody, or a trispecific binding fragment thereof, or a pharmaceutical composition comprising the same,
wherein the trispecific antibody, or the trispecific binding fragment thereof, comprises:
(a) a first antigen-binding arm comprising a first heavy chain variable domain (VH1) and a first light chain variable domain (VL1), wherein the VH1 and VL1 comprise the HCDR1, the HCDR2, the HCDR3, the LCDR1, the LCDR2, and the LCDR3 of:
a) SEQ ID NOs: 1, 2, 3, 4, 5 and 6, respectively;
b) SEQ ID NOs: 13, 8, 9, 10, 11 and 12, respectively;
c) SEQ ID NOs: 7, 8, 9, 10, 11 and 12, respectively;
d) SEQ ID NOs: 14, 15, 16, 17, 5 and 6, respectively;
e) SEQ ID NOs: 18, 8, 19, 20, 21 and 12, respectively;
f) SEQ ID NOs: 22, 23, 24, 25, 5 and 6, respectively;
g) SEQ ID NOs: 22, 26, 27, 28, 5 and 29, respectively; or
h) SEQ ID NOs: 30, 31, 32, 33, 5 and 6, respectively;
(b) a second antigen-binding arm comprising a second heavy chain variable domain (VH2) and a second light chain variable domain (VL2), wherein the VH2 and VL2 comprise the HCDR1, the HCDR2, the HCDR3, the LCDR1, the LCDR2, and the LCDR3 of:
a) SEQ ID NOs: 76, 77, 78, 79, 80 and 81, respectively;
b) SEQ ID NOs: 76, 77, 75, 79, 80 and 81, respectively;
c) SEQ ID NOs: 76, 77, 82, 79, 80 and 81, respectively; or
d) SEQ ID NOs: 83, 84, 85, 86, 87 and 88, respectively;
(c) a third antigen-binding arm comprising a third heavy chain variable domain (VH3) and a third light chain variable domain (VL3), wherein the VH3 and VL3 comprise the HCDR1, the HCDR2, the HCDR3, the LCDR1, the LCDR2, and the LCDR3 of:
a) SEQ ID NOs: 115, 116, 117, 118, 119 and 120, respectively;
b) SEQ ID NOs: 121, 122, 123, 124, 119 and 125, respectively;
c) SEQ ID NOs: 115, 116, 95, 96, 119 and 125, respectively; or
d) SEQ ID NOs: 121, 116, 123, 124, 119 and 125, respectively,
wherein the first antigen-binding arm binds to an epitope on cluster of differentiation 79B protein (CD79b), the second antigen-binding arm binds to an epitope on cluster of differentiation 3 (CD3), and the third antigen-binding arm binds to an epitope on cluster of differentiation 20 (CD20).

55. The method of claim 54, wherein the cancer cell is a B cell cancer cell.

56. The method of claim 55, wherein the B cell cancer cell is from B-cell lymphoma or a non-Hodgkin lymphoma.

57. The method of claim 54, wherein the trispecific antibody or trispecific binding fragment, or the pharmaceutical composition is administered intravenously, intramuscularly, intraperitoneally, or subcutaneously to the subject.

58. A method of redirecting a T cell to CD79b and/or CD20-expressing cancer cells in a subject in need thereof, said method comprising administering to said subject a therapeutically effective amount of a trispecific antibody, or a trispecific binding fragment thereof, or a pharmaceutical composition comprising the same,
wherein the trispecific antibody, or the trispecific binding fragment thereof, comprises:
(a) a first antigen-binding arm comprising a first heavy chain variable domain (VH1) and a first light chain variable domain (VL1), wherein the VH1 and VL1 comprise the HCDR1, the HCDR2, the HCDR3, the LCDR1, the LCDR2, and the LCDR3 of:
a) SEQ ID NOs: 1, 2, 3, 4, 5 and 6, respectively;
b) SEQ ID NOs: 13, 8, 9, 10, 11 and 12, respectively;
c) SEQ ID NOs: 7, 8, 9, 10, 11 and 12, respectively;
d) SEQ ID NOs: 14, 15, 16, 17, 5 and 6, respectively;
e) SEQ ID NOs: 18, 8, 19, 20, 21 and 12, respectively;
f) SEQ ID NOs: 22, 23, 24, 25, 5 and 6, respectively;
g) SEQ ID NOs: 22, 26, 27, 28, 5 and 29, respectively; or
h) SEQ ID NOs: 30, 31, 32, 33, 5 and 6, respectively;
(b) a second antigen-binding arm comprising a second heavy chain variable domain (VH2) and a second light chain variable domain (VL2), wherein the VH2 and VL2 comprise the HCDR1, the HCDR2, the HCDR3, the LCDR1, the LCDR2, and the LCDR3 of:
a) SEQ ID NOs: 76, 77, 78, 79, 80 and 81, respectively;
b) SEQ ID NOs: 76, 77, 75, 79, 80 and 81, respectively;
c) SEQ ID NOs: 76, 77, 82, 79, 80 and 81, respectively; or
d) SEQ ID NOs: 83, 84, 85, 86, 87 and 88, respectively;
(c) a third antigen-binding arm comprising a third heavy chain variable domain (VH3) and a third light chain variable domain (VL3), wherein the VH3 and VL3 comprise the HCDR1, the HCDR2, the HCDR3, the LCDR1, the LCDR2, and the LCDR3 of:
a) SEQ ID NOs: 115, 116, 117, 118, 119 and 120, respectively;
b) SEQ ID NOs: 121, 122, 123, 124, 119 and 125, respectively;
c) SEQ ID NOs: 115, 116, 95, 96, 119 and 125, respectively; or
d) SEQ ID NOs: 121, 116, 123, 124, 119 and 125, respectively,
wherein the first antigen-binding arm binds to an epitope on cluster of differentiation 79B protein (CD79b), the second antigen-binding arm binds to an epitope on cluster of differentiation 3 (CD3), and the third antigen-binding arm binds to an epitope on cluster of differentiation 20 (CD20).

59. The method of claim 58, wherein the CD79b and/or CD20-expressing cancer cell is a B cell cancer cell.

60. The method of claim 59, wherein the B cell cancer cell is from B-cell lymphoma or a non-Hodgkin lymphoma.

61. The method of claim 58, wherein the trispecific antibody or trispecific binding fragment, or the pharmaceutical composition is administered intravenously, intramuscularly, intraperitoneally, or subcutaneously to the subject.

62. A trispecific antibody or trispecific binding fragment thereof, comprising:
(a) a first antigen-binding arm comprising a first heavy chain variable domain (VH1) and a first light chain variable domain (VL1), wherein the VH1 and VL1 comprise an HCDR1, an HCDR2, an HCDR3, an LCDR1, an LCDR2, and an LCDR3, wherein the first antigen-binding arm comprises:
(i) the HCDR1, the HCDR2 and the HCDR3 of the VH1 of SEQ ID NO: 35 and the LCDR1, the LCDR2 and the LCDR3 of the VL1 of SEQ ID NO: 37;
(ii) the HCDR1, the HCDR2, the HCDR3, the LCDR1, the LCDR2 and the LCDR3 of SEQ ID NOs: 1, 2, 3, 4, 5 and 6, respectively; and/or
(iii) the VH1 of SEQ ID NO: 35 and the VL1 of SEQ ID NO: 37;
(b) a second antigen-binding arm comprising a second heavy chain variable domain (VH2) and a second light chain variable domain (VL2), wherein the VH2 and VL2 comprise an HCDR1, an HCDR2, an HCDR3, an LCDR1, an LCDR2, and an LCDR3, wherein the second antigen-binding arm comprises:
(i) the HCDR1, the HCDR2 and the HCDR3 of the VH2 of SEQ ID NO: 101 and the LCDR1, the LCDR2 and the LCDR3 of the VL2 of SEQ ID NO: 99;
(ii) the HCDR1, the HCDR2, the HCDR3, the LCDR1, the LCDR2 and the LCDR3 of SEQ ID NOs: 76, 77, 75, 79, 80 and 81, respectively; and/or
(iii) the VH2 of SEQ ID NO: 101 and the VL2 of SEQ ID NO: 99; and
(c) a third antigen-binding arm comprising a third heavy chain variable domain (VH3) and a third light chain variable domain (VL3), wherein the VH3 and VL3 comprise an HCDR1, an HCDR2, an HCDR3, an LCDR1, an LCDR2, and an LCDR3, wherein the third antigen-binding arm comprises:
(i) the HCDR1, the HCDR2 and the HCDR3 of the VH3 of SEQ ID NO: 130 and the LCDR1, the LCDR2 and the LCDR3 of the VL3 of SEQ ID NO: 132;
(ii) the HCDR1, the HCDR2, the HCDR3, the LCDR1, the LCDR2 and the LCDR3 of SEQ ID NOs: 121, 122, 123, 124, 119 and 125, respectively; and/or
(iii) the VH3 of SEQ ID NO: 130 and the VL3 of SEQ ID NO: 132;
wherein the first antigen-binding arm binds to an epitope on cluster of differentiation 79B protein (CD79b), the second antigen-binding arm binds to an epitope on cluster of differentiation 3 (CD3), and the third antigen-binding arm binds to an epitope on cluster of differentiation 20 (CD20).
